(19) United States
(12) Patent Application Publication
(10) Pub. No.: US 2024/0409893 A1
(43) Pub. Date: Dec. 12, 2024

(54) CAR T CELLS GENERATED BY EFFECTOR PROTEINS AND METHODS RELATED THERETO

(71) Applicant: Mammoth Biosciences, Inc., Brisbane, CA (US)

(72) Inventors: Subhadra JAYARAMAN RUKMINI, Redwood City, CA (US); Pei-Qi LIU, Oakland, CA (US); Lucas Benjamin HARRINGTON, San Francisco, CA (US); Pooja KYATSANDRA NARENDRA, Belmont, CA (US)

(21) Appl. No.: 18/732,352

(22) Filed: Jun. 3, 2024

Related U.S. Application Data
(63) Continuation of application No. PCT/US2022/081042, filed on Dec. 6, 2022.
(60) Provisional application No. 63/286,993, filed on Dec. 7, 2021.
(60) Provisional application No. 63/371,507, filed on Aug. 15, 2022.

(51) Int. Cl. C12N 5/0783; C07K 16/28; C12N 9/22; C12N 15/11; C12N 15/90

(52) U.S. Cl. CPC C12N 5/0636; C07K 16/2803; C12N 9/22; C12N 15/11; C12N 15/907; C07K 2317/73; C07K 2319/00; C12N 2310/20; C12N 2510/00

(57) ABSTRACT

Provided herein are viral vectors comprising nucleotide sequences for production of an effector protein, guide nucleic acids for targeting modification of select genes to abrogate allogeneic immune reactions of T cells, and a donor nucleic acid encoding a chimeric antigen receptor (CAR), and uses thereof. Due to the small nature of the effector proteins provided herein, the viral vectors provided herein have ample room for all needed components for the efficient and robust production of CAR T cells from allogeneic donors. Various compositions, systems, and methods of the present disclosure leverage the activities of these effector proteins for the generation of “off-the-self” CAR T cells.

### CROSS REFERENCE TO RELATED APPLICATIONS

This application is a continuation of International Patent Application No. PCT/US2022/081042, filed Dec. 6, 2022, which claims the benefit of priority of U.S. Provisional Application No. 63/286,993, filed Dec. 7, 2021, and U.S. Provisional Application No. 63/371,507, filed Aug. 15, 2022, the disclosures of which are incorporated herein by reference in their entirety.

### SEQUENCE LISTING

The instant application contains a Sequence Listing, which has been submitted via Patent Center. The Sequence Listing titled 203477-704301US_ST26.xml, which was created on May 29, 2024, and is 2,754,572 bytes in size, is hereby incorporated by reference in its entirety.

### FIELD

The present disclosure relates generally to chimeric antigen receptor (CAR) T cells (CAR T cells) generated by effector proteins, and more specifically to CAR T cells generated by contacting a T cell with a viral vector encoding an effector protein, guide nucleic acids targeting the T-cell receptor alpha-constant (TRAC) gene, the beta-2 microglobulin (B2M) gene and class II major histocompatibility complex transactivator (CIITA gene), and a donor nucleic acid encoding the CAR.

### BACKGROUND

Programmable nucleases are proteins that bind and cleave nucleic acids in a sequence-specific manner with the assistance of a guide nucleic acid. A programmable nuclease, such as a CRISPR-associated (Cas) protein, may be coupled to a guide nucleic acid that imparts activity or sequence selectivity to the programmable nuclease. The programmable nuclease and guide nucleic acid form a complex that recognizes a target region of a nucleic acid and cleaves the nucleic acid within the target region or at a position adjacent to the target region.

Guide nucleic acids, sometimes referred to as a CRISPR RNA (crRNA), include a nucleotide sequence that is at least partially complementary to a target nucleic acid. Guide nucleic acids can include additional nucleic acids that impact the activity of the programmable nuclease, which include a trans-activating crRNA (tracrRNA) sequence, at least a portion of which interacts with the programmable nuclease. Alternatively, a tracrRNA can be provided separately from the guide nucleic acid. The tracrRNA may, in some instances, hybridize to a portion of the guide nucleic acid that does not hybridize to the target nucleic acid.

Programmable nucleases may cleave a variety of nucleic acids in a variety of ways. For example, a programmable nuclease may cleave a single stranded RNA (ssRNA), a double stranded DNA (dsDNA), or a single-stranded DNA (ssDNA). Additionally, programmable nucleases may provide a cis cleavage activity, a trans cleavage activity, a nickase activity, or a combination such activities. Cis cleavage activity is often described as cleavage of a target nucleic acid that is hybridized to a guide nucleic acid, wherein cleavage occurs within or directly adjacent to the region of the target nucleic acid that is hybridized to guide nucleic acid. Trans cleavage activity (sometimes referred to as transcollateral cleavage), is often described as cleavage of ssDNA or ssRNA that is near, but not hybridized to the guide nucleic acid. Trans cleavage activity can be triggered by the hybridization of a guide nucleic acid to the target nucleic acid. Nickase activity is typically described as the selective cleavage of one strand of a dsDNA molecule.

Although complexes of programmable nucleases and guide nucleic acids are quite flexible in modifying a target nucleic acid, in order for many programmable nucleases to be used therapeutically, such as, for genome editing, they must be efficiently delivered to a target cell, which often means they must be packaged in an appropriate manner to be delivered to a target cell or subject. In some instances, that delivery may include genetically modifying a therapeutic cell, such as a T lymphocyte (T cell), that will be delivered to the subject. Recombinant adeno-associated virus (AAV) vectors are useful delivery platforms for therapeutic genome editing. However, if the AAV vector is loaded with too much cargo (e.g., genome editing components totaling more than 4.5 kb in length), viral production becomes compromised. For example, if the sequence encoding the genome editing tools included a region encoding a Cas9 protein, which is ˜4 kb, a guide nucleic acid, and respective promoters, there would be no substantial space remaining for a donor nucleic acid.

Selective targeting of T cells by introduction of a chimeric antigen receptor (CAR), which allows for predetermined antigen specific recognition and activation of the T cells in an HLA-independent matter, has become one of the leading areas of development for adoptive immunotherapy, especially in the adoptive cancer immunotherapy setting. However, one of the major limitations of this therapy is a lack of patient compatible T cells.

Allogeneic donors can be an abundant source of T cells for generating therapeutic CAR T cells, and sometimes are required for treating certain patients, such as an immunodeficient patient. However, use of such T cells presents its own challenges. For example, CAR T cells generated from an allogenic donor T cell can result in graft-versus-host disease (GVHD) when transplanted to a patient, which is induced by donor-derived allogeneic T cells recognizing host-derived normal tissues through their endogenous T-cell receptor (TCR). GVHD can be acute GVHD or chronic GVHD, and lead to loss of therapeutic cells, risk of damage to a number of organs or tissues and even death. Moreover, current in vitro preparation of autologous T cells can be rather laborious and cost intensive, and the quality of the cells can vary.

Therefore, there is a need for efficient and consistent production of therapeutically sufficient and functional antigen-specific T cells for adoptive immunotherapies. The present disclosure satisfies this need and provides related advantages.

### SUMMARY

Provided herein, in some aspects, is a viral vector comprising: a) a first nucleotide sequence that encodes an effector protein; b) a second nucleotide sequence that, when transcribed and/or cleaved by the effector protein, produces a first guide nucleic acid, wherein the first guide nucleic acid comprises a sequence that is at least 90% identical or complementary to an equal length portion of a target sequence of a gene encoding human T-cell receptor alpha-constant (TRAC gene); c) a third nucleotide sequence that, when transcribed and/or cleaved by the effector protein, produces a second guide nucleic acid, wherein the second guide nucleic acid comprises a sequence that is at least 90% identical or complementary to an equal length portion of a target sequence of a gene encoding human beta-2 microglobulin (B2M gene); d) a fourth nucleotide sequence that, when transcribed and/or cleaved by the effector protein, produces a third guide nucleic acid, wherein the third guide nucleic acid comprises a sequence that is at least 90% identical or complementary to an equal length portion of a target sequence of a gene encoding human class II major histocompatibility complex transactivator (CIITA gene); and e) a fifth nucleotide sequence that comprises a donor nucleic acid, wherein the donor nucleic acid encodes a chimeric antigen receptor (CAR) and comprises one or more nucleotide sequences for directing integration into the TRAC gene, wherein each of the first guide nucleic acid, the second guide nucleic acid, and the third guide nucleic acid comprise a nucleotide sequence that the effector protein binds.

In some embodiments, a viral vector provided herein comprises a nucleotide sequence that encodes an effector protein, wherein the effector protein comprises an amino acid sequence described herein. In some embodiments, the effector protein comprises an amino acid sequence that has at least about 85% sequence identity to a sequence selected from the group consisting of SEQ ID NOs: 1-45 or 2435. In some embodiments, the effector protein comprises an amino acid sequence that has at least about 90% sequence identity to a sequence selected from the group consisting of SEQ ID NOs: 1-45 or 2435. In some embodiments, the effector protein comprises an amino acid sequence that has at least about 95% sequence identity to a sequence selected from the group consisting of SEQ ID NOs: 1-45 or 2435. In some embodiments, the effector protein comprises an amino acid sequence that has at least about 98% sequence identity to a sequence selected from the group consisting of SEQ ID NOs: 1-45 or 2435. In some embodiments, the effector protein comprises an amino acid sequence that has at least about 99% sequence identity to a sequence selected from the group consisting of SEQ ID NOs: 1-45 or 2435. In some embodiments, the effector protein comprises an amino acid sequence that is identical to a sequence selected from the group consisting of SEQ ID NOs: 1-45 or 2435. In some embodiments, the effector protein comprises an amino acid sequence that has at least about 85% sequence identity to a sequence selected from the group consisting of SEQ ID NOs: 46-94. In some embodiments, the effector protein comprises an amino acid sequence that has at least about 90% sequence identity to a sequence selected from the group consisting of SEQ ID NOs: 46-94. In some embodiments, the effector protein comprises an amino acid sequence that has at least about 95% sequence identity to a sequence selected from the group consisting of SEQ ID NOs: 46-94. In some embodiments, the effector protein comprises an amino acid sequence that has at least about 98% sequence identity to a sequence selected from the group consisting of SEQ ID NOs: 46-94. In some embodiments, the effector protein comprises an amino acid sequence that has at least about 99% sequence identity to a sequence selected from the group consisting of SEQ ID NOs: 46-94. In some embodiments, the effector protein comprises an amino acid sequence that is identical to a sequence selected from the group consisting of SEQ ID NOs: 46-94. In some embodiments, the effector protein comprises an amino acid sequence that has at least about 85%, at least about 90%, at least about 95%, at least about 98%, at least about 99% sequence identity, or is identical, to a sequence selected from the group consisting of SEQ ID NOs: 95-203.

In some embodiments, a viral vector provided herein comprises a nucleotide sequence that encodes an effector protein, wherein the effector protein comprises an amino acid sequence of a specified length. In some embodiments, the effector protein comprises an amino acid sequence length that is less than about 600, less than about 500, less than about 450 amino acids, or less than about 400 amino acids. In some embodiments, the effector protein comprises an amino acid sequence length that is at least about 300, at least about 350, at least about 400, or at least about 450 linked amino acids. In some embodiments, the effector protein comprises an amino acid sequence length that is about 300 to about 600 linked amino acids. In some embodiments, the effector protein comprises an amino acid sequence length that is about 400 to about 600 linked amino acids. In some embodiments, the effector protein comprises an amino acid sequence length that is about 450 to about 500 linked amino acids. In some embodiments, the effector protein comprises an amino acid sequence length that is about 420 to about 480 linked amino acids.

In some embodiments, a viral vector provided herein comprises a nucleotide sequence that, when transcribed and/or cleaved by the effector protein, produces a guide nucleic acid that has one or more features described herein. In some embodiments, the first guide nucleic acid, the second guide nucleic acid, and the third guide nucleic acid are each a guide RNA. In some embodiments, the nucleotide sequence that the effector protein binds is the same for the first guide nucleic acid, the second guide nucleic acid, and the third guide nucleic acid. In some embodiments, the nucleotide sequence that the effector protein binds is different for the first guide nucleic acid, the second guide nucleic acid, and the third guide nucleic acid. In some embodiments, the nucleotide sequence that the effector protein binds for the first guide nucleic acid, the second guide nucleic acid, and the third guide nucleic acid comprise at least about 90%, at least about 95%, at least about 98%, or at least 99% sequence identity to each other. In some embodiments, any one of the first guide nucleic acid, the second guide nucleic acid, and the third guide nucleic acid comprise a tracrRNA sequence. In some embodiments, the tracrRNA sequence comprises the nucleotide sequence of any one of SEQ ID NO: 385-440. In some embodiments, the first guide nucleic acid comprises a sequence that is complementary to an equal length portion of a target sequence of the TRAC gene. In some embodiments, the second guide nucleic acid comprises a sequence that is complementary to an equal length portion of a target sequence of the B2M gene. In some embodiments, the third guide nucleic acid comprises a sequence that is complementary to an equal length portion of a target sequence of the CIITA gene. In some embodiments, the first guide nucleic acid comprises a sequence that is at least 90% identical to an equal length portion of a target sequence of the TRAC gene. In some embodiments, the second guide nucleic acid comprises a sequence that is at least 90% identical to an equal length portion of a target sequence of the B2M gene. In some embodiments, the third guide nucleic acid comprises a sequence that is at least 90% identical to an equal length portion of a target sequence of the CIITA gene. In some embodiments, the first guide nucleic acid comprises a nucleotide sequence of any one of the sequences recited in TABLE 5, TABLE 5.1, TABLE 9, TABLE 9.1, TABLE 10, TABLE 10.1, TABLE 14, and TABLE 14.1. In some embodiments, the second guide nucleic acid comprises a nucleotide sequence of any one of the sequences recited in TABLE 6, TABLE 6.1, TABLE 11, TABLE 11.1, TABLE 12, TABLE 12.1, TABLE 15, and TABLE 15.1. In some embodiments, the third guide nucleic acid comprises a nucleotide sequence of any one of the sequences recited in TABLE 7, TABLE 7.1, TABLE 8, TABLE 13, and TABLE 16.

In some embodiments, a viral vector provided herein comprises at least one promoter that drives expression of the first guide nucleic acid, the second guide nucleic acid, the third guide nucleic acid, the effector protein, or a combination thereof. In some embodiments, a viral vector provided herein comprises a first promoter that drives expression of the first guide nucleic acid, the second guide nucleic acid, and the third guide nucleic acid as a single RNA transcript, and a second promoter that drives expression of the effector protein. In some embodiments, a viral vector provided herein comprises a first promoter that drives expression of the first guide nucleic acid, a second promoter that drives expression of the second guide nucleic acid, a third promoter that drives expression of the third guide nucleic acid, and a fourth promoter that drives expression of the effector protein.

In some embodiments, a viral vector provided herein comprises a fifth nucleotide sequence that comprises a donor nucleic acid, wherein the donor nucleic acid encodes a CAR, and wherein the CAR binds to an antigen expressed by a cancer cell. In some embodiments, the antigen is selected from the group consisting of ADRB3, AKAP-4,ALK, Androgen receptor, B7H3, BCMA, BORIS, BST2, CAIX, CD 179a, CD123, CD171, CD19, CD20, CD22, CD24, CD30, CD300LF, CD33, CD38, CD44v6, CD72, CD79a, CD79b, CD97, CEA, CLDN6, CLEC12A, CLL-1, CS-1, CXORF61, CYP1B1, Cyclin B 1, E7, EGFR, EGFRvIII, ELF2M, EMR2, EPCAM, ERBB2 (Her2/neu), ERG (TMPRSS2 ETS fusion gene), ETV6-AML, EphA2, Ephrin B2, FAP, FCAR, FCRL5, FLT3, Folate receptor alpha, Folate receptor beta, Fos-related antigen 1, Fucosyl GMl, GD2, GD3, GM3, GPC3, GPR20, GPRC5D, GloboH, HAVCR1, HMWMAA, HPV E6, IGF-I receptor, IL-13Ra2, IL-11Ra, KIT, LAGE-1a, LAIR1, LCK, LILRA2, LMP2, LY6K, LY75, LewisY, MAD-CT-1, MAD-CT-2, MAGE A1, MAGE-A1, ML-IAP, MUC1, MYCN, MelanA/MARTl, Mesothelin, NA17, NCAM, NY-BR-1, NY-ESO-1, OR51E2, OY- TES 1, PANX3, PAP, PAX3, PAX5, PCTA-1/Galectin 8, PDGFR-beta, PLAC1, PRSS21, PSCA, PSMA, Polysialic acid, Prostase, RAGE-1, ROR1, RU1, RU2, Ras mutant, RhoC, SART3, SSEA-4, SSX2, TAG72, TARP, TEM1/CD248, TEM7R, TGS5, TRP-2, TSHR, Tie 2, Tn Ag, UPK2, VEGFR2, WT1, XAGE1, and IGLL1.

In some embodiments, a viral vector provided herein comprises two inverted terminal repeats of an AAV.

Provided herein, in some aspects, is a viral particle comprising a viral vector described herein. In some embodiments, the viral particle is a retrovirus, an adenovirus, an arenavirus, an alphavirus, an AAV, a baculovirus, a vaccinia virus, a herpes simplex virus or a poxvirus. In some embodiments, the viral particle is an AAV.

Provided herein, in some aspects, is a pharmaceutical composition comprising a viral vector or a viral particle described herein and a pharmaceutically acceptable excipient, carrier or diluent.

Provided herein, in some aspects, is a method of producing an immunologically compatible CAR T cell comprising: a) contacting ex vivo a T cell with a viral vector described herein, a viral particle described herein, or a pharmaceutical composition described herein for a sufficient period of time to allow for viral transduction of the T cell; and b) culturing the T cell for a sufficient period of time for indels to occur in the TRAC gene, B2M gene and CIITA gene and for integration of the donor nucleic acid into the TRAC gene, thereby producing the immunologically compatible CAR T cell. In some embodiments, the contacting ex vivo comprises at least about 1 hour, at least about 2 hours, at least about 3 hours, at least about 4 hours, at least about 5 hours or at least about 6 hours. In some embodiments, the method comprises using a multiplicity of infection (MOI) of viral vector or viral particle to T cell of about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, about 1×10⁹, about 5×10⁹, about 1×10¹⁰, or about 5×10¹⁰. In some embodiments, the culturing is for at least 1 day, at least 2 days, at least 3 days, at least 4 days, at least 5 days, or at least 6 days. In some embodiments, the culturing is for no more than 7 days, no more than 8 days, no more than 9 days, no more than 10 days, no more than 11 days, no more than 12 days, no more than 13 days, no more than 14 days, no more than 15 days, no more than 16 days, no more than 17 days, no more than 18 days, no more than 19 days, no more than 20 days, no more than 21 days. In some embodiments, the method further comprises freezing the CAR T-cell. In some embodiments, the method comprises no other agent that alters the CAR T-cell's ability to recognize a target cell or pathogen or autoreactivity of the CAR T-cell in a subject. In some embodiments, the indels prevent expression of human T-cell receptor alpha-constant, human beta-2 microglobulin, and human class II major histocompatibility complex transactivator.

Provided herein, in some aspects, is a method of producing a population of immunologically compatible CAR T cells comprising: a) contacting ex vivo a population of T cells with a viral vector described here, a viral particle described herein, or a pharmaceutical described herein for a sufficient period of time to allow for viral transduction of T cells contained in the population; and b) culturing the population of T cells for sufficient period of time for indels to occur in the TRAC gene, B2M gene and CIITA gene and for integration of the donor nucleic acid into the TRAC gene in at least 50% of the T cells contained in the population, thereby producing the population of immunologically compatible CAR T cells. In some embodiments, the contacting ex vivo comprises at least about 1 hour, at least about 2 hours, at least about 3 hours, at least about 4 hours, at least about 5 hours or at least about 6 hours. In some embodiments, the method comprises a MOI of viral vector or viral particle to T cell of T cell of about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, about 1×10⁹, about 5×10⁹, about 1×10¹⁰, or about 5×10¹⁰. In some embodiments, the culturing is for at least 1 day, at least 2 days, at least 3 days, at least 4 days, at least 5 days, or at least 6 days. In some embodiments, the culturing is for no more than 7 days, no more than 8 days, no more than 9 days, no more than 10 days, no more than 11 days, no more than 12 days, no more than 13 days, no more than 14 days, no more than 15 days, no more than 16 days, no more than 17 days, no more than 18 days, no more than 19 days, no more than 20 days, or no more than 21 days. In some embodiments, the method comprises no other agent that alters the T cells′, contained in the population, ability to recognize a target cell or pathogen or autoreactivity of the T cells contained in the population in a subject. In some embodiments, the period of time is sufficient for at least 55% of the T cells contained in the population to have indels occur in the TRAC gene, B2M gene and CIITA gene and integration of the donor nucleic acid. In some embodiments, the period of time is sufficient for at least 60% of the T cells contained in the population to have indels occur in the TRAC gene, B2M gene and CIITA gene and integration of the donor nucleic acid. In some embodiments, the period of time is sufficient for at least 65% of the T cells contained in the population to have indels occur in the TRAC gene, B2M gene and CIITA gene and integration of the donor nucleic acid. In some embodiments, the period of time is sufficient for at least 75% of the T cells contained in the population to have indels occur in the TRAC gene, B2M gene and CIITA gene and integration of the donor nucleic acid. In some embodiments, the period of time is sufficient for at least 80% of the T cells contained in the population to have indels occur in of TRAC gene, B2M gene and CIITA gene and integration of the donor nucleic acid. In some embodiments, the number of T cells that are killed during the method is no more than 1% based on the number of T cells present in the population at the start of the method. In some embodiments, the number of T cells that are killed during the method is no more than 3% based on the number of T cells present in the population at the start of the method. In some embodiments, the number of T cells that are killed during the method is no more than 5% based on the number of T cells present in the population at the start of the method. In some embodiments, the number of T cells that are killed during the method is no more than 10% based on the number of T cells present in the population at the start of the method. In some embodiments, the number of T cells that are killed during the method is no more than 15% based on the number of T cells present in the population at the start of the method. In some embodiments, the method further comprises freezing the population of T cells. In some embodiments, the indels prevent expression of human T-cell receptor alpha-constant, human beta-2 microglobulin, and human class II major histocompatibility complex transactivator.

Provided herein, in some aspects, is a method of producing an immunologically compatible CAR T cell comprising: a) contacting ex vivo a T cell with a viral vector or viral particle comprising a donor nucleic acid encoding the CAR for a sufficient period of time to allow for viral transduction of the T cell; b) contacting ex vivo the T cell with at least three different ribonucleoprotein (RNP) complexes comprising an effector protein and a guide nucleic acid, wherein the at least three RNP complexes comprise: i. an effector protein and a first guide nucleic acid, wherein the first guide nucleic acid comprises a sequence that is at least 90% identical or complementary to an equal length portion of a target sequence of a gene encoding human T-cell receptor alpha-constant (TRAC gene); ii. an effector protein and a second guide nucleic acid, wherein the second guide nucleic acid comprises a sequence that is at least 90% identical or complementary to an equal length portion of a target sequence of a gene encoding human beta-2 microglobulin (B2M gene); iii. an effector protein and a third guide nucleic acid, wherein the third guide nucleic acid comprises a sequence that is at least 90% identical or complementary to an equal length portion of a target sequence of a gene encoding human class II major histocompatibility complex transactivator (CIITA gene); and c) culturing the T cell for a sufficient period of time for indels to occur in the TRAC gene, B2M gene and CIITA gene and for integration of the donor nucleic acid into the TRAC gene, thereby producing the immunologically compatible CAR T cell.

In some embodiments, a method provided herein comprises use of a viral vector, a viral particle or a pharmaceutical composition described herein, wherein the viral vector comprises a nucleotide sequence that encodes an effector protein, wherein the effector protein comprises an amino acid sequence described herein. In some embodiments, the effector protein comprises an amino acid sequence that has at least about 85% sequence identity to a sequence selected from the group consisting of SEQ ID NOs: 1-45 or 2435. In some embodiments, the effector protein comprises an amino acid sequence that has at least about 90% sequence identity to a sequence selected from the group consisting of SEQ ID NOs: 1-45 or 2435. In some embodiments, the effector protein comprises an amino acid sequence that has at least about 95% sequence identity to a sequence selected from the group consisting of SEQ ID NOs: 1-45 or 2435. In some embodiments, the effector protein comprises an amino acid sequence that has at least about 98% sequence identity to a sequence selected from the group consisting of SEQ ID NOs: 1-45 or 2435. In some embodiments, the effector protein comprises an amino acid sequence that has at least about 99% sequence identity to a sequence selected from the group consisting of SEQ ID NOs: 1-45 or 2435. In some embodiments, the effector protein comprises an amino acid sequence that is identical to a sequence selected from the group consisting of SEQ ID NOs: 1-45 or 2435. In some embodiments, the effector protein comprises an amino acid sequence that has at least about 85% sequence identity to a sequence selected from the group consisting of SEQ ID NOs: 46-94. In some embodiments, the effector protein comprises an amino acid sequence that has at least about 90% sequence identity to a sequence selected from the group consisting of SEQ ID NOs: 46-94. In some embodiments, the effector protein comprises an amino acid sequence that has at least about 95% sequence identity to a sequence selected from the group consisting of SEQ ID NOs: 46-94. In some embodiments, the effector protein comprises an amino acid sequence that has at least about 98% sequence identity to a sequence selected from the group consisting of SEQ ID NOs: 46-94. In some embodiments, the effector protein comprises an amino acid sequence that has at least about 99% sequence identity to a sequence selected from the group consisting of SEQ ID NOs: 46-94. In some embodiments, the effector protein comprises an amino acid sequence that is identical to a sequence selected from the group consisting of SEQ ID NOs: 46-94. In some embodiments, the effector protein comprises an amino acid sequence that has at least about 85%, at least about 90%, at least about 95%, at least about 98%, at least about 99% sequence identity, or is identical, to a sequence selected from the group consisting of SEQ ID NOs: 95-203.

In some embodiments, a method provided herein comprises use of a viral vector, a viral particle or a pharmaceutical composition described herein, the viral vector comprises a nucleotide sequence that encodes an effector protein, wherein the effector protein comprises an amino acid sequence of a specified length. In some embodiments, the effector protein comprises an amino acid sequence length that is less than about 600, less than about 500, less than about 450 amino acids, or less than about 400 amino acids. In some embodiments, the effector protein comprises an amino acid sequence length that is at least about 300, at least about 350, at least about 400, or at least about 450 linked amino acids. In some embodiments, the effector protein comprises an amino acid sequence length that is about 300 to about 600 linked amino acids. In some embodiments, the effector protein comprises an amino acid sequence length that is about 400 to about 600 linked amino acids. In some embodiments, the effector protein comprises an amino acid sequence length that is about 450 to about 500 linked amino acids. In some embodiments, the effector protein comprises an amino acid sequence length that is about 420 to about 480 linked amino acids.

In some embodiments, a method provided herein comprises use of a viral vector, a viral particle or a pharmaceutical composition described herein, wherein the viral vector comprises a nucleotide sequence that, when transcribed and/or cleaved by the effector protein, produces a guide nucleic acid that has one or more features described herein. In some embodiments, the first guide nucleic acid, the second guide nucleic acid, and the third guide nucleic acid are each a guide RNA. In some embodiments, the nucleotide sequence that the effector protein binds is the same for the first guide nucleic acid, the second guide nucleic acid, and the third guide nucleic acid. In some embodiments, the nucleotide sequence that the effector protein binds is different for the first guide nucleic acid, the second guide nucleic acid, and the third guide nucleic acid. In some embodiments, the nucleotide sequences that the effector protein bind for the first guide nucleic acid, the second guide nucleic acid, and the third guide nucleic acid comprise at least about 90%, at least about 95%, at least about 98%, or at least 99% sequence identity to each other. In some embodiments, the first guide nucleic acid, the second guide nucleic acid, and the third guide nucleic acid comprise a tracrRNA sequence. In some embodiments, the tracrRNA sequence comprises the nucleotide sequence of any one of SEQ ID NO: 385-440. In some embodiments, the first guide nucleic acid comprises a sequence that is complementary to an equal length portion of a target sequence of the TRAC gene. In some embodiments, the second guide nucleic acid comprises a sequence that is complementary to an equal length portion of a target sequence of the B2M gene. In some embodiments, wherein the third guide nucleic acid comprises a sequence that is complementary to an equal length portion of a target sequence of the CIITA gene. In some embodiments, the first guide nucleic acid comprises a sequence that is at least 90% identical to an equal length portion of a target sequence of the TRAC gene. In some embodiments, the second guide nucleic acid comprises a sequence that is at least 90% identical to an equal length portion of a target sequence of the B2M gene. In some embodiments, the third guide nucleic acid comprises a sequence that is at least 90% identical to an equal length portion of a target sequence of the CIITA gene. In some embodiments, the first guide nucleic acid comprises the nucleotide sequence of any one of the sequences recited in TABLE 5, TABLE 5.1, TABLE 9, TABLE 9.1, TABLE 10, TABLE 10.1, TABLE 14, and TABLE 14.1. In some embodiments, the second guide nucleic acid comprises the nucleotide sequence of any one of the sequences recited in TABLE 6, TABLE 6.1, TABLE 11, TABLE 11.1, TABLE 12, TABLE 12.1, TABLE 15, and TABLE 15.1. In some embodiments, the third guide nucleic acid comprises the nucleotide sequence of any one of the sequences recited in TABLE 7, TABLE 7.1, TABLE 8, TABLE 13, and TABLE 16.

In some embodiments, a method provided herein comprises use of a viral vector, a viral particle or a pharmaceutical composition described herein, wherein the viral vector provided herein comprises a fifth nucleotide sequence that comprises a donor nucleic acid, wherein the donor nucleic acid encodes a CAR, and wherein the CAR binds to an antigen expressed by a cancer cell. In some embodiments, the antigen is selected from the group consisting of ADRB3, AKAP-4,ALK, Androgen receptor, B7H3, BCMA, BORIS, BST2, CAIX, CD 179a, CD123, CD171, CD19, CD20, CD22, CD24, CD30, CD300LF, CD33, CD38, CD44v6, CD72, CD79a, CD79b, CD97, CEA, CLDN6, CLEC12A, CLL-1, CS-1, CXORF61, CYP1B1, Cyclin B 1, E7, EGFR, EGFRvIII, ELF2M, EMR2, EPCAM, ERBB2 (Her2/neu), ERG (TMPRSS2 ETS fusion gene), ETV6-AML, EphA2, Ephrin B2, FAP, FCAR, FCRL5, FLT3, Folate receptor alpha, Folate receptor beta, Fos-related antigen 1, Fucosyl GMl, GD2, GD3, GM3, GPC3, GPR20, GPRC5D, GloboH, HAVCR1, HMWMAA, HPV E6, IGF-I receptor, IL-13Ra2, IL-11Ra, KIT, LAGE-1a, LAIR1, LCK, LILRA2, LMP2, LY6K, LY75, LewisY, MAD-CT-1, MAD-CT-2, MAGE A1, MAGE-A1, ML-IAP, MUC1, MYCN, MelanA/MARTl, Mesothelin, NA17, NCAM, NY-BR-1, NY-ESO-1, OR51E2, OY- TES 1, PANX3, PAP, PAX3, PAX5, PCTA-1/Galectin 8, PDGFR-beta, PLAC1, PRSS21, PSCA, PSMA, Polysialic acid, Prostase, RAGE-1, ROR1, RU1, RU2, Ras mutant, RhoC, SART3, SSEA-4, SSX2, TAG72, TARP, TEM1/CD248, TEM7R, TGS5, TRP-2, TSHR, Tie 2, Tn Ag, UPK2, VEGFR2, WT1, XAGE1, and IGLL1.

In some embodiments, a method provided herein comprises use of a viral vector, a viral particle or a pharmaceutical composition described herein, wherein the viral vector provided herein comprises two inverted terminal repeats of is an AAV. In some embodiments, the method comprises contacting with the viral particle. In some embodiments, the viral particle is a retrovirus, an adenovirus, an arenavirus, an alphavirus, an AAV, a baculovirus, a vaccinia virus, a herpes simplex virus or a poxvirus. In some embodiments, the viral particle is an AAV.

In some embodiments, a method provided herein comprises contacting ex vivo a T cell with a viral vector or viral particle comprising a donor nucleic acid encoding the CAR for a sufficient period of time to allow for viral transduction of the T cell, wherein the contacting ex vivo comprises at least about 1 hour, at least about 2 hours, at least about 3 hours, at least about 4 hours, at least about 5 hours or at least about 6 hours. In some embodiments, the method comprises using a MOI of viral vector or viral particle to T cell of T cell of about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, about 1×10⁹, about 5×10⁹, about 1×10¹⁰, or about 5×10¹⁰.

In some embodiments, a method provided herein comprises culturing the T cell for a sufficient period of time for indels to occur in the TRAC gene, B2M gene and CIITA gene and for integration of the donor nucleic acid into the TRAC gene, the culturing is for at least 1 day, at least 2 days, at least 3 days, at least 4 days, at least 5 days, or at least 6 days. In some embodiments, the culturing is for no more than 7 days, no more than 8 days, no more than 9 days, no more than 10 days, no more than 11 days, no more than 12 days, no more than 13 days, no more than 14 days, no more than 15 days, no more than 16 days, no more than 17 days, no more than 18 days, no more than 19 days, no more than 20 days, no more than 21 days.

In some embodiments, a method provided herein further comprises freezing the T cell. In some embodiments, a method provided herein comprises no other agent that alters the T cell's ability to recognize a target cell or pathogen or autoreactivity of the T cell in a subject. In some embodiments, a method provided herein comprises culturing the T cell for a sufficient period of time for indels to occur in the TRAC gene, B2M gene and CIITA gene and for integration of the donor nucleic acid into the TRAC gene, wherein the indels prevent expression of human T-cell receptor alpha-constant, human beta-2 microglobulin, and human class II major histocompatibility complex transactivator. In some embodiments, a method provided herein comprises contacting ex vivo the T cell with at least three different RNP complexes comprising an effector protein and a guide nucleic acid, wherein contacting ex vivo the T cell with at least three different RNP complexes comprises electroporation, lipofection, or lipid nanoparticle (LNP) delivery of the RNP complexes.

Provided herein, in some aspects, is a method of producing a population of immunologically compatible CAR T cells comprising: a) contacting ex vivo a population of T cells with a viral vector or viral particle comprising a donor nucleic acid encoding the CAR for a sufficient period of time to allow for viral transduction of T cells contained in the population; b) contacting ex vivo the population of T cells with at least three different RNP complexes comprising an effector protein and a guide nucleic acid, wherein the at least three RNP complexes comprise: i. an effector protein and a first guide nucleic acid, wherein the first guide nucleic acid comprises a sequence that is at least 90% identical or complementary to an equal length portion of a target sequence of a gene encoding human T-cell receptor alpha-constant (TRAC gene); ii. an effector protein and a second guide nucleic acid, wherein the second guide nucleic acid comprises a sequence that is at least 90% identical or complementary to an equal length portion of a target sequence of a gene encoding human beta-2 microglobulin (B2M gene); iii. an effector protein and a third guide nucleic acid, wherein the third guide nucleic acid comprises a sequence that is at least 90% identical or complementary to an equal length portion of a target sequence of a gene encoding human class II major histocompatibility complex transactivator (CIITA gene); and c) culturing the population of T cells for sufficient period of time for indels to occur in the TRAC gene, B2M gene and CIITA gene and for integration of the donor nucleic acid into the TRAC gene in at least 50% of the T cells contained in the population of T cells, thereby producing the population of CAR T cells.

In some embodiments, a method provided herein comprises use of RNP complexes comprising an effector protein, wherein the effector protein comprises an amino acid sequence described herein. In some embodiments, the effector protein comprises an amino acid sequence that has at least about 85% sequence identity to a sequence selected from the group consisting of SEQ ID NOs: 1-45 or 2435. In some embodiments, the effector protein comprises an amino acid sequence that has at least about 90% sequence identity to a sequence selected from the group consisting of SEQ ID NOs: 1-45 or 2435. In some embodiments, the effector protein comprises an amino acid sequence that has at least about 95% sequence identity to a sequence selected from the group consisting of SEQ ID NOs: 1-45 or 2435. In some embodiments, the effector protein comprises an amino acid sequence that has at least about 98% sequence identity to a sequence selected from the group consisting of SEQ ID NOs: 1-45 or 2435. In some embodiments, the effector protein comprises an amino acid sequence that has at least about 99% sequence identity to a sequence selected from the group consisting of SEQ ID NOs: 1-45 or 2435. In some embodiments, the effector protein comprises an amino acid sequence that is identical to a sequence selected from the group consisting of SEQ ID NOs: 1-45 or 2435. In some embodiments, the effector protein comprises an amino acid sequence that has at least about 85% sequence identity to a sequence selected from the group consisting of SEQ ID NOs: 46-94. In some embodiments, the effector protein comprises an amino acid sequence that has at least about 90% sequence identity to a sequence selected from the group consisting of SEQ ID NOs: 46-94. In some embodiments, the effector protein comprises an amino acid sequence that has at least about 95% sequence identity to a sequence selected from the group consisting of SEQ ID NOs: 46-94. In some embodiments, the effector protein comprises an amino acid sequence that has at least about 98% sequence identity to a sequence selected from the group consisting of SEQ ID NOs: 46-94. In some embodiments, the effector protein comprises an amino acid sequence that has at least about 99% sequence identity to a sequence selected from the group consisting of SEQ ID NOs: 46-94. In some embodiments, the effector protein comprises an amino acid sequence that is identical to a sequence selected from the group consisting of SEQ ID NOs: 46-94. In some embodiments, the effector protein comprises an amino acid sequence that has at least about 85%, at least about 90%, at least about 95%, at least about 98%, at least about 99% sequence identity, or is identical, to a sequence selected from the group consisting of SEQ ID NOs: 95-203.

In some embodiments, a method provided herein comprises use of RNP complexes comprising an effector protein, wherein the effector protein comprises an amino acid sequence of a specified length. In some embodiments, the effector protein comprises an amino acid sequence length that is less than about 600, less than about 500, less than about 450 amino acids, or less than about 400 amino acids. In some embodiments, the effector protein comprises an amino acid sequence length that is at least about 300, at least about 350, at least about 400, or at least about 450 linked amino acids. In some embodiments, the effector protein comprises an amino acid sequence length that is about 300 to about 600 linked amino acids. In some embodiments, the effector protein comprises an amino acid sequence length that is about 400 to about 600 linked amino acids. In some embodiments, the effector protein comprises an amino acid sequence length that is about 450 to about 500 linked amino acids. In some embodiments, the effector protein comprises an amino acid sequence length that is about 420 to about 480 linked amino acids.

In some embodiments, a method provided herein comprises use of RNP complexes comprising a guide nucleic acid having one or more features described herein. In some embodiments, the first guide nucleic acid, the second guide nucleic acid, and the third guide nucleic acid are each a guide RNA. In some embodiments, the nucleotide sequence that the effector protein binds is the same for the first guide nucleic acid, the second guide nucleic acid, and the third guide nucleic acid. In some embodiments, the nucleotide sequence that the effector protein binds is different for the first guide nucleic acid, the second guide nucleic acid, and the third guide nucleic acid. In some embodiments, the nucleotide sequences that the effector protein bind for the first guide nucleic acid, the second guide nucleic acid, and the third guide nucleic acid comprise at least about 90%, at least about 95%, at least about 98%, or at least 99% sequence identity to each other. In some embodiments, the first guide nucleic acid, the second guide nucleic acid, and the third guide nucleic acid comprise a tracrRNA sequence. In some embodiments, the tracrRNA sequence comprises the nucleotide sequence of any one of SEQ ID NO: 385-440. In some embodiments, the first guide nucleic acid comprises a sequence that is complementary to an equal length portion of a target sequence of the TRAC gene. In some embodiments, the second guide nucleic acid comprises a sequence that is complementary to an equal length portion of a target sequence of the B2M gene. In some embodiments, the third guide nucleic acid comprises a sequence that is complementary to an equal length portion of a target sequence of the CIITA gene. In some embodiments, the first guide nucleic acid comprises a sequence that is at least 90% identical to an equal length portion of a target sequence of the TRAC gene. In some embodiments, the second guide nucleic acid comprises a sequence that is at least 90% identical to an equal length portion of a target sequence of the B2M gene. In some embodiments, the third guide nucleic acid comprises a sequence that is at least 90% identical to an equal length portion of a target sequence of the CIITA gene. In some embodiments, the first guide nucleic acid comprises the nucleotide sequence of any one of the sequences recited in TABLE 5, TABLE 5.1, TABLE 9, TABLE 9.1, TABLE 10, TABLE 10.1, TABLE 14, and TABLE 14.1. In some embodiments, the second guide nucleic acid comprises the nucleotide sequence of any one of the sequences recited in TABLE 6, TABLE 6.1, TABLE 11, TABLE 11.1, TABLE 12, TABLE 12.1, TABLE 15, and TABLE 15.1. In some embodiments, the third guide nucleic acid comprises the nucleotide sequence of any one of the sequences recited in TABLE 7, TABLE 7.1, TABLE 8, TABLE 13, and TABLE 16.

In some embodiments, a method provided herein comprises use of a viral vector or a viral particle comprising a donor nucleic acid, wherein the donor nucleic acid encodes a CAR, and wherein the CAR binds to an antigen expressed by a cancer cell. In some embodiments, the antigen is selected from the group consisting of ADRB3, AKAP-4,ALK, Androgen receptor, B7H3, BCMA, BORIS, BST2, CAIX, CD 179a, CD123, CD171, CD19, CD20, CD22, CD24, CD30, CD300LF, CD33, CD38, CD44v6, CD72, CD79a, CD79b, CD97, CEA, CLDN6, CLEC12A, CLL-1, CS-1, CXORF61, CYP1B1, Cyclin B 1, E7, EGFR, EGFRvIII, ELF2M, EMR2, EPCAM, ERBB2 (Her2/neu), ERG (TMPRSS2 ETS fusion gene), ETV6-AML, EphA2, Ephrin B2, FAP, FCAR, FCRL5, FLT3, Folate receptor alpha, Folate receptor beta, Fos-related antigen 1, Fucosyl GMl, GD2, GD3, GM3, GPC3, GPR20, GPRC5D, GloboH, HAVCR1, HMWMAA, HPV E6, IGF-I receptor, IL-13Ra2, IL-11Ra, KIT, LAGE-1a, LAIR1, LCK, LILRA2, LMP2, LY6K, LY75, LewisY, MAD-CT-1, MAD-CT-2, MAGE A1, MAGE-A1, ML-IAP, MUC1, MYCN, MelanA/MARTl, Mesothelin, NA17, NCAM, NY-BR-1, NY-ESO-1, OR51E2, OY- TES 1, PANX3, PAP, PAX3, PAX5, PCTA-1/Galectin 8, PDGFR-beta, PLAC1, PRSS21, PSCA, PSMA, Polysialic acid, Prostase, RAGE-1, ROR1, RU1, RU2, Ras mutant, RhoC, SART3, SSEA-4, SSX2, TAG72, TARP, TEM1/CD248, TEM7R, TGS5, TRP-2, TSHR, Tie 2, Tn Ag, UPK2, VEGFR2, WT1, XAGE1, and IGLL1.

In some embodiments, a method provided herein comprises use of a viral vector or a viral particle described herein, wherein viral vector comprises two inverted terminal repeats of an AAV. In some embodiments, the method comprises contacting with the viral particle. In some embodiments, the viral particle is a retrovirus, an adenovirus, an arenavirus, an alphavirus, an AAV, a baculovirus, a vaccinia virus, a herpes simplex virus or a poxvirus. In some embodiments, the viral particle is an AAV.

In some embodiments, a method provided herein comprises contacting ex vivo a population of T cells with a viral vector or viral particle comprising a donor nucleic acid encoding the CAR for a sufficient period of time to allow for viral transduction of the T cell, wherein the contacting ex vivo comprises at least about 1 hour, at least about 2 hours, at least about 3 hours, at least about 4 hours, at least about 5 hours or at least about 6 hours. In some embodiments, the method comprises a MOI of viral vector or viral particle to T cell of about 1×10⁴, about 5×10⁴, about 1×10⁴, about 5×10⁴, about 1×10⁶, about 5×10⁶, about 1×10⁷, about 5×10⁷, about 1×10⁸, about 5×10⁸, about 1×10⁹, about 5×10⁹, about 1×10¹⁰, or about 5×10¹⁰.

In some embodiments, a method provided herein comprises culturing a population of T cells for sufficient period of time for indels to occur in the TRAC gene, B2M gene and CIITA gene and for integration of the donor nucleic acid into the TRAC gene in at least 50% of the T cells contained in the population of T cells, wherein the culturing is for at least 1 day, at least 2 days, at least 3 days, at least 4 days, at least 5 days, or at least 6 days. In some embodiments, the culturing is for no more than 7 days, no more than 8 days, no more than 9 days, no more than 10 days, no more than 11 days, no more than 12 days, no more than 13 days, no more than 14 days, no more than 15 days, no more than 16 days, no more than 17 days, no more than 18 days, no more than 19 days, no more than 20 days, no more than 21 days. In some embodiment, the period of time is sufficient for at least 55% of the T cells contained in the population to have indels occur in the TRAC gene, B2M gene and CIITA gene and integration of the donor nucleic acid. In some embodiment, the period of time is sufficient for at least 60% of the T cells contained in the population to have indels occur in the TRAC gene, B2M gene and CIITA gene and integration of the donor nucleic acid. In some embodiment, the period of time is sufficient for at least 65% of the T cells contained in the population to have indels occur in the TRAC gene, B2M gene and CIITA gene and integration of the donor nucleic acid. In some embodiment, the period of time is sufficient for at least 75% of the T cells contained in the population to have indels occur in the TRAC gene, B2M gene and CIITA gene and integration of the donor nucleic acid. In some embodiment, the period of time is sufficient for at least 80% of the T cells contained in the population to have indels occur in the TRAC gene, B2M gene and CIITA gene and integration of the donor nucleic acid.

In some embodiments, the method of producing a population of immunologically compatible CAR T cells provided herein comprises no other agent that alters the T cells′, contained in the population, ability to recognize a target cell or pathogen or autoreactivity of the T cells contained in the population in a subject. In some embodiments, the method comprises contacting ex vivo the population of T cells with at least three different RNP complexes comprises electroporation, lipofection, or lipid nanoparticle (LNP) delivery of the RNP complexes. In some embodiments, the method further comprises freezing the population of T cells. In some embodiments, the method comprises culturing the population of T cells for sufficient period of time for indels to occur in the TRAC gene, B2M gene and CIITA gene and for integration of the donor nucleic acid into the TRAC gene in at least 50% of the T cells contained in the population of T cells, wherein the indels prevent expression of human T-cell receptor alpha-constant, human beta-2 microglobulin, and human class II major histocompatibility complex transactivator. In some embodiments, the number of T cells that are killed during the method is no more than 1% based on the number of T cells present in the population at the start of the method. In some embodiments, the number of T cells that are killed during the method is 3% based on the number of T cells present in the population at the start of the method. In some embodiments, the number of T cells that are killed during the method is 5% based on the number of T cells present in the population at the start of the method. In some embodiments, the number of T cells that are killed during the method is 10% based on the number of T cells present in the population at the start of the method. In some embodiments, the number of T cells that are killed during the method is 15% based on the number of T cells present in the population at the start of the method.

Provided herein, in some aspects, is an immunologically compatible CAR T cell made by a method described herein.

Provided herein, in some aspects, is a population of immunologically compatible CAR T cells made by a method described herein.

Provided herein, in some aspects, is an immunologically compatible CART cell comprising: a) indels in each of a human T-cell receptor alpha-constant (TRAC gene), human beta-2 microglobulin (B2M gene), and human class II major histocompatibility complex transactivator (CIITA gene), wherein each of the indels is within proximity of a protospacer adjacent motif (PAM) sequence of an effector protein; and b) integration of a donor nucleic acid encoding a CAR into the TRAC gene. In some embodiments, the PAM sequence comprises 5′-CTT-3′, 5′-CC-3′, 5′-TCG-3′, 5′-GCG-3′, 5′-TTG-3′, 5′-GTG-3′, 5′-ATTA-3′, 5′-ATTG-3′, 5′-GTTA-3′, 5′-GTTG-3′, 5′-TC-3′, 5′-ACTG-3′, 5′-GCTG-3′, 5′-TTC-3′, or 5′-TTT-3′. In some embodiments, the PAM sequence comprises 5′-TBN-3′, wherein B is one or more of C, G, or T and N is any nucleotide. In some embodiments, the PAM sequence comprises 5′-TTTN-3′. In some embodiments, PAM sequence comprises 5′-GTTK-3′, 5′-VTTK-3′, 5′-VTTS-3′, 5′-TTTS-3′ or 5′-VTTN-3′, wherein K is G or T, V is A, C or G, S is C or G, and N is any nucleotide. In some embodiments, the indels are within 10 nucleotides of the PAM sequence. In some embodiments, the indels are within 15 nucleotides of the PAM sequence. In some embodiments, the indels are within 20 nucleotides of the PAM sequence. In some embodiments, the indels are within 25 nucleotides of the PAM sequence. In some embodiments, the indels are within 30 nucleotides of the PAM sequence. In some embodiments, the CAR T cell is a cytotoxic T cell or a helper T cell. In some embodiments, expression of the donor nucleic acid is driven by an endogenous TRAC gene promotor of the T cell.

Provided herein, in some aspects, is a population of T cells comprising an immunologically compatible CART cell described herein. In some embodiments, at least 50% of the T cells contained in the population are an immunologically compatible CAR T cell described herein. In some embodiments, at least 55% of the T cells contained in the population are an immunologically compatible CAR T cell described herein. In some embodiments, at least 60% of the T cells contained in the population are an immunologically compatible CAR T cell described herein. In some embodiments, at least 65% of the T cells contained in the population are an immunologically compatible CAR T cell described herein. In some embodiments, at least 70% of the T cells contained in the population are an immunologically compatible CAR T cell described herein. In some embodiments, at least 75% of the T cells contained in the population are an immunologically compatible CAR T cell described herein. In some embodiments, at least 80% of the T cells contained in the population are an immunologically compatible CAR T cell described herein. In some embodiments, the CAR T cell is a cytotoxic T cell or a helper T cell.

Provided herein, in some aspects, is a kit for making an immunologically compatible CAR T cell comprising: a) a viral vector described herein or a viral particle described herein; and b) one or more reagents for transducing a T cell. In some embodiments, the kit further comprises one or more containers comprising the viral vector and the one or more reagents. In some embodiments, the kit further comprises a package, carrier, or container that is compartmentalized to receive the one or more containers.

Provided herein, in some aspects, is a system comprising a T cell and a viral vector described or a viral particle described herein.

Provided herein, in some aspects, is a method for killing a cell or pathogen in a subject comprising administering an effective amount of an immunologically compatible CAR T cell described herein or a population of immunologically compatible CAR T cells described herein to the subject.

Provided herein, in some aspects, is method for killing a cell or pathogen in a subject comprising: a) obtaining T cells from a first subject; b) performing a method described herein; and c) administering an effective amount of the immunologically compatible CAR T cells back to the first subject or to a second subject. In some embodiments, obtaining the T cells and administering an effective amount of the immunologically compatible CAR T cells is for a period of time that is no more than 21 days. In some embodiments, obtaining the T cells and administering an effective amount of the immunologically compatible CAR T cells is for a period of time that is no more than 20 days. In some embodiments, obtaining the T cells and administering an effective amount of the immunologically compatible CAR T cells is for a period of time that is no more than 19 days. In some embodiments, obtaining the T cells and administering an effective amount of the immunologically compatible CAR T cells is for a period of time that is no more than 18 days. In some embodiments, obtaining the T cells and administering an effective amount of the immunologically compatible CAR T cells is for a period of time that is no more than 17 days. In some embodiments, obtaining the T cells and administering an effective amount of the immunologically compatible CAR T cells is no more than 16 days. In some embodiments, obtaining the T cells and administering an effective amount of the immunologically compatible CAR T cells is for a period of time that is no more than 15 days. In some embodiments, obtaining the T cells and administering an effective amount of the immunologically compatible CAR T cells is for a period of time that is no more than 14 days. In some embodiments, obtaining the T cells and administering an effective amount of the immunologically compatible CAR T cells is for a period of time that is no more than 13 days. In some embodiments, obtaining the T cells and administering an effective amount of the immunologically compatible CAR T cells is for a period of time that is no more than 12 days. In some embodiments, obtaining the T cells and administering an effective amount of the immunologically compatible CAR T cells is for a period of time that is no more than 11 days. In some embodiments, obtaining the T cells and administering an effective amount of the immunologically compatible CAR T cells is for a period of time that is no more than 10 days. In some embodiments, obtaining the T cells and administering an effective amount of the immunologically compatible CAR T cells is for a period of time that is no more than 9 days. In some embodiments, obtaining the T cells and administering an effective amount of the immunologically compatible CAR T cells is for a period of time that is no more than 8 days. In some embodiments, obtaining the T cells and administering an effective amount of the immunologically compatible CAR T cells is for a period of time that is no more than 7 days. In some embodiments, obtaining the T cells and administering an effective amount of the immunologically compatible CAR T cells is for a period of time that is no more than 6 days. In some embodiments, the T cells obtained from the first subject is a naïve T cell. In some embodiments, the CAR T cell administered to the first or second subject is a cytotoxic T cell or a helper T cell.

Provided herein, in some aspects, is a method of reducing tumor size in a subject comprising administering an effective amount of an CAR T cell described herein or a population of CAR T cells described herein to the subject.

Provided herein, in some aspects, is a method of reducing tumor size in a subject comprising: a) obtaining T cells from a first subject; b) performing a method described herein; and c) administering an effective amount of the immunologically compatible CAR T cells back to the first subject or a second subject. In some embodiments, obtaining the T cells and administering an effective amount of the immunologically compatible CAR T cells is for a period of time that is no more than 21 days. In some embodiments, obtaining the T cells and administering an effective amount of the immunologically compatible CAR T cells is for a period of time that is no more than 20 days. In some embodiments, obtaining the T cells and administering an effective amount of the immunologically compatible CAR T cells is for a period of time that is no more than 19 days. In some embodiments, obtaining the T cells and administering an effective amount of the immunologically compatible CAR T cells is for a period of time that is no more than 18 days. In some embodiments, obtaining the T cells and administering an effective amount of the immunologically compatible CAR T cells is for a period of time that is no more than 17 days. In some embodiments, obtaining the T cells and administering an effective amount of the immunologically compatible CAR T cells is for a period of time that is no more than 16 days. In some embodiments, obtaining the T cells and administering an effective amount of the immunologically compatible CAR T cells is for a period of time that is no more than 15 days. In some embodiments, obtaining the T cells and administering an effective amount of the immunologically compatible CAR T cells is for a period of time that is no more than 14 days. In some embodiments, obtaining the T cells and administering an effective amount of the immunologically compatible CAR T cells is for a period of time that is no more than 13 days. In some embodiments, obtaining the T cells and administering an effective amount of the immunologically compatible CAR T cells is for a period of time that is no more than 12 days. In some embodiments, obtaining the T cells and administering an effective amount of the immunologically compatible CAR T cells is for a period of time that is no more than 11 days. In some embodiments, obtaining the T cells and administering an effective amount of the immunologically compatible CAR T cells is for a period of time that is no more than 10 days. In some embodiments, obtaining the T cells and administering an effective amount of the immunologically compatible CAR T cells is for a period of time that is no more than 9 days. In some embodiments, obtaining the T cells and administering an effective amount of the immunologically compatible CAR T cells is for a period of time that is no more than 8 days. In some embodiments, obtaining the T cells and administering an effective amount of the immunologically compatible CAR T cells is for a period of time that is no more than 7 days. In some embodiments, obtaining the T cells and administering an effective amount of the immunologically compatible CAR T cells is for a period of time that is no more than 6 days. In some embodiments, the T cells obtained from the first subject is a naïve T cell. In some embodiments, the CAR T cell administered to the first or second subject is a cytotoxic T cell or a helper T cell.

Also provided herein are viral vectors comprising: a first nucleotide sequence that encodes an effector protein; and a second nucleotide sequence that, when transcribed and/or cleaved by the effector protein, produces a guide nucleic acid. In some embodiments, the guide nucleic acid comprises a sequence that is at least 90% identical or complementary to an equal length portion of a target sequence of a gene encoding human T-cell receptor alpha-constant (TRAC gene). In some embodiments, the guide nucleic acid comprises a nucleotide sequence that the effector protein binds. In some embodiments, the effector protein comprises a sequence with at least about: 90%, 95%, 96%, 97%, 98%, or 99% sequence homology to SEQ ID NO: 2435. In some embodiments, the effector protein comprises the amino acid sequence of SEQ ID NO: 2435. In some embodiments, the guide nucleic acid comprises a sequence with at least about: 90%, 95%, 96%, 97%, 98%, or 99% sequence identity to any one of the sequences recited in TABLE 5, TABLE 5.1, TABLE 9, TABLE 9.1, TABLE 10, TABLE 10.1, TABLE 14, and TABLE 14.1. In some embodiments, the guide nucleic acid comprises any one of the sequences recited in TABLE 5, TABLE 5.1, TABLE 9, TABLE 9.1, TABLE 10, TABLE 10.1, TABLE 14, and TABLE 14.1. In some embodiments, the viral vector is an scAAV vector. In some embodiments, the viral vector is an ssAAV vector. Also provided herein are T-cells comprising the viral vector. In some embodiments, the T-cells comprise cytotoxic T cells or helper T cells.

Also provided herein are viral vectors comprising: a first nucleotide sequence that encodes an effector protein; and a second nucleotide sequence that, when transcribed and/or cleaved by the effector protein, produces a guide nucleic acid. In some embodiments, the guide nucleic acid comprises a sequence that is at least 90% identical or complementary to an equal length portion of a target sequence of a gene encoding human beta-2 microglobulin (B2M gene). In some embodiments, the guide nucleic acid comprises a nucleotide sequence that the effector protein binds. In some embodiments, the effector protein comprises a sequence with at least about: 90%, 95%, 96%, 97%, 98%, or 99% sequence homology to SEQ ID NO: 2435. In some embodiments, the effector protein comprises the amino acid sequence of SEQ ID NO: 2435. In some embodiments, the guide nucleic acid comprises a sequence with at least about: 90%, 95%, 96%, 97%, 98%, or 99% sequence identity to any one of the sequences recited in TABLE 6, TABLE 6.1, TABLE 11, TABLE 11.1, TABLE 12, TABLE 12.1, TABLE 15, and TABLE 15.1. In some embodiments, the guide nucleic acid comprises any one of the sequences recited in TABLE 6, TABLE 6.1, TABLE 11, TABLE 11.1, TABLE 12, TABLE 12.1, TABLE 15, and TABLE 15.1. In some embodiments, the viral vector is an scAAV vector. In some embodiments, the viral vector is an ssAAV vector. Also provided herein are T-cells comprising the viral vector. In some embodiments, the T-cells comprise cytotoxic T cells or helper T cells.

Also provided herein are viral vectors comprising: a first nucleotide sequence that encodes an effector protein; and a second nucleotide sequence that, when transcribed and/or cleaved by the effector protein, produces a guide nucleic acid. In some embodiments, the guide nucleic acid comprises a sequence that is at least 90% identical or complementary to an equal length portion of a target sequence of a gene encoding human class II major histocompatibility complex transactivator (CIITA gene). In some embodiments, the guide nucleic acid comprises a nucleotide sequence that the effector protein binds. In some embodiments, the effector protein comprises a sequence with at least about: 90%, 95%, 96%, 97%, 98%, or 99% sequence homology to SEQ ID NO: 2435. In some embodiments, the effector protein comprises the amino acid sequence of SEQ ID NO: 2435. In some embodiments, the guide nucleic acid comprises a sequence with at least about: 90%, 95%, 96%, 97%, 98%, or 99% sequence identity to anyone of the sequences recited in TABLE 7, TABLE 7.1, TABLE 8, TABLE 13, and TABLE 16. In some embodiments, the guide nucleic acid comprises any one of the sequences recited in TABLE 7, TABLE 7.1, TABLE 8, TABLE 13, and TABLE 16. In some embodiments, the viral vector is an scAAV vector. In some embodiments, the viral vector is an ssAAV vector. Also provided herein are T-cells comprising the viral vector. In some embodiments, the T-cells comprise cytotoxic T cells or helper T cells.

Also provided herein are methods of producing a population of immunologically compatible chimeric antigen receptor (CAR) T cells comprising: contacting ex vivo a population of T cells with a viral vector described herein, for a sufficient period of time to allow for viral transduction of T cells contained in the population; and culturing the population of T cells for sufficient period of time for indels to occur in the TRAC gene, the B2M gene or the CIITA gene, thereby producing the population of immunologically compatible CAR T cells.

### BRIEF DESCRIPTION OF THE DRAWINGS

FIG. 1 shows exemplary AAV vectors encoding small Cas effectors compared to an AAV vector encoding a Cas9 protein.

FIG. 2 shows the frequency of indel mutations generated in the PCSK9 gene in Hepal-6 cells with AAV vector encoding CasΦ.12 and a guide RNA.

FIG. 3 shows that a plasmid encoding a guide RNA and a Cas effector protein having a length of between 400 and 500 amino acids can edit the genome of mammalian cells.

FIG. 4 shows that a plasmid encoding a guide RNA and a Cas effector protein having a length of between 400 and 500 amino acids can edit the genome of mammalian cells at multiple doses.

FIGS. 5A-5D illustrate the PAM requirement of CasΦ polypeptides. FIG. 5A shows the PAM requirement of CasΦ.2, CasΦ.4, CasΦ.11 and CasΦ.12. FIG. 5B shows the PAM requirement of CasΦ.20, CasΦ.26, CasΦ.32, CasΦ.38 and CasΦ.45. FIG. 5C shows the cleavage products from the assessment of the PAM requirement for CasΦ.20, CasΦ.24 and CasΦ.25. FIG. 5D shows the quantification of the raw data shown in FIG. 5C.

FIGS. 6A-6F illustrate endogenous gene editing in primary cells. FIG. 6A shows a flow cytometry analysis of T cells that have received CasΦ.12 with or without a gRNA targeting the beta-2 microglobulin gene. FIG. 6B shows the modification detected in K562 cells and T cells following delivery of CasΦ.12 and a gRNA targeting the beta-2 microglobulin gene. FIG. 6C shows the sequence analysis of the T cell population which received CasΦ.12 and the gRNA targeting the beta-2 microglobulin gene. FIG. 6D shows a flow cytometry analysis of T cells that have received CasΦ.12 with a gRNA targeting the T Cell Receptor Alpha Constant gene. FIG. 6E shows the sequence analysis of cell populations that received CasΦ.12 with a gRNA targeting the T Cell Receptor Alpha Constant gene. FIG. 6F shows the quantification of indels detected by sequence analysis.

FIGS. 7A-7B illustrate the CasΦ.12-mediated efficiency is comparable to that of Cas9. FIG. 7A shows the frequency of indel mutations and quantification of B2M knockout cells from flow cytometry panels in FIG. 7B.

FIGS. 8A-8E illustrate the ability of CasΦ.12 to target B2M and TRAC genes. FIG. 8A shows the percentage of B2M and TRAC knockout after CasΦ.12-mediated genome editing with gRNAs with a repeat length of 20 nucleotides and a spacer length of 20 nucleotides. FIG. 8B shows the percentage of B2M and TRAC knockout after CasΦ.12-mediated genome editing with gRNAs with a repeat length of 20 nucleotides and a spacer length of 17 nucleotides. FIG. 8C shows corresponding flow cytometry panels for B2M and TRAC knockout with different gRNAs. FIG. 8D shows the percentage of TRAC knockout after CasΦ.12-mediated genome editing with modified gRNAs of different spacer lengths (repeat length of 20 nucleotides and a spacer length of 17 or 20 nucleotides). FIG. 8E shows a corresponding flow cytometry panel for TRAC knockout after CasΦ.12-mediated genome editing.

FIGS. 9A-9E illustrate exemplary gRNAs for targeting TRAC, B2M and PD1 with CasΦ.12 in human primary T cells.

FIG. 9F shows the screening of gRNAs targeting TRAC.

FIG. 9H shows the screening of gRNAs targeting B2M.

FIGS. 9G and 9I show flow cytometry panels of exemplary gRNAs targeting TRAC and B2M, respectively.

FIGS. 10A-10J illustrate delivery of CasΦ.12 RNPs or CasΦ.12 mRNA both lead to efficient genome editing of B2M and TRAC in T cells as compared to Cas9. FIG. 10A and FIG. 10B show flow cytometry panels of CasΦ.12 RNP complexes targeting B2M and TRAC in T cells, and are quantified in FIG. 10C and FIG. 10D. FIG. 10E and FIG. 10F show the quantification of indels detected by sequence analysis with delivery of CasΦ.12 RNPs. FIG. 10G and FIG. 10I show the frequency of indel mutations after delivery of CasΦ.12 mRNA as compared to Cas9. FIG. 10H shows an exemplary FACS panel for two data points in FIG. 10G used to quantify B2M knockout cells. FIG. 10J shows the distribution of the size of indel mutations induced by CasΦ.12 or Cas9. No indel is denoted at “0” on the indel size.

FIG. 11 illustrates the ability of CasΦ RNP complexes to knockout multiple genes simultaneously. T cells were nucleofected with RNP complexes of CasΦ.12 and gRNAs targeting B2M, TRAC or PDCD1 and the percentage knockout was measured using flow cytometry.

FIGS. 12A-12G illustrate the ability of a CasΦ.12 all-in-one vector to mediate genome editing in Hepal-6 mouse hepatoma cells. FIG. 12A shows a plasmid map of the AAV encoding the CasΦ polypeptide sequence and gRNA sequence. FIG. 12B illustrates repeat truncations. FIG. 12C shows various truncated repeat sequences (25 nt, 20 nt and 19 nt), the data of which shown in FIGS. 12D-12G. FIG. 12D shows efficient transfection with AAV. FIG. 12E shows the frequency of CasΦ.12 induced indel mutations. FIG. 12F and FIG. 12G show the frequency of CasΦ.12 induced indel mutations with different gRNA containing repeat and spacer sequences of different lengths.

FIG. 13 illustrates the optimization of LNP delivery of mRNA encoding CasΦ and gRNA. A range of N/P ratios were tested and the frequency of indel mutations was determined.

FIG. 14 illustrates CasΦ-mediated genome editing of the CIITA locus in K562 cells. Cells were nucleofected with RNP complexes (CasΦ polypeptides and gRNAs targeting CIITA) and the frequency of indel mutations was determined by NGS.

FIG. 15 illustrates PAM preferences for different effector proteins disclosed herein. Frequency of nucleotides at each PAM position was independently calculated using a position frequency matrix (PFM) and plotted as a WebLogo. The number at the top of the plot corresponds to the composition number of TABLE 3 and TABLE 4, denoting the effector protein used, as well as the combination of crRNA, sgRNA, and/or tracrRNA sequence.

FIG. 16 shows exemplary dose dependent cytotoxicity of CD19-CAR T cells to CD19+ NALM6 cells. Ratio of Effector Cells:Target Cells assayed included 1:1 and 5:1. Controls include GFP and T cell only.

FIG. 17 shows exemplary dose dependent cytotoxicity of CD19-CAR T cells to CD19+ NALM6 cells. Ratio of Effector Cells:Target Cells assayed included 0.5:1, 1:1, and 5:1. Control is T cell only.

FIG. 18 show FACS results of B2M editing in primary T cells at day 3 post electroporation for the percent of B2M negative cells with different amounts of Cas 265466 and different amounts of guide constructs.

FIG. 19 shows editing of TRAC in primary T cells with different amounts of Cas 265466 and different amounts of guide constructs. The graph shows sequencing results at day 3 post electroporation of the percent indels in TRAC in primary T cells treated with different amounts of Cas 265466 and different amounts of guide constructs.

FIG. 20 shows editing of CIITA in primary T cells with different amounts of Cas 265466 and different amounts of guide constructs. The graph shows sequencing results at day 3 post electroporation of the percent indels in CIITA in primary T cells treated with different amounts of Cas 265466 and different amounts of guide constructs.

FIG. 21 shows editing of B2M in primary NK cells with Cas 265466 and different guide constructs. The graph shows sequencing results at day 3 post electroporation of the percent indels in B2M in primary NK cells treated with Cas 265466 and different guide constructs. Different electroporation conditions were tested to identify conditions for NK cell electroporation.

FIG. 22 shows editing of B2M in primary T cells with Cas 265466 and a guide construct in an scAAV vector. The graph shows sequencing results post transduction of the percent indels in B2M in primary T cells treated with Cas 265466 and a guide construct.

FIG. 23 shows exemplary schematics of scAAV construct for gene editing according to one or more embodiments of the present disclosure. Included in FIG. 23 are the following abbreviations representing elements of the AAV construct: gRNA=guide RNA; P1=first promoter; P2=second promoter; Cas=effector protein.

FIG. 24 shows the frequency of indel mutations generated in primary T cells with AAV vector encoding Cas19952 and a guide RNA at a ranging from 5e+02 to 5e+05.

FIGS. 25A-25B illustrates results of CasΦ.12 L26R mediated CD19 integration in T cells. FIG. 25A shows FACS analysis of T cells treated with an RNP complex of CasΦ.12 L26R effector protein and a guide RNA having a sequence of SEQ ID NO: 2593, wherein treated T cells were incubated with CD3 antibody to identify the portion of the treated T cells that have the TRAC gene knocked out. Absence of CD3 protein on surface of the treated T cells indicates successful knock out of the TRAC gene. FIG. 25B shows FACS analysis of T cells treated with the RNP complex and AAV6 particles containing a donor nucleotide sequence encoding a GFP marker. GFP expression indicates successfully GFP marker integration into a TRAC gene locus.

FIG. 26 illustrates results of % indel generated by CasΦ.12 L26R effector proteins.

FIGS. 27A-27B illustrates results of CasΦ.12 L26R mediated CD19 integration in T cells. FIG. 27A shows FACS analysis of T cells treated with an RNP complex of CasΦ.12 L26R effector protein and a guide RNA having a sequence of SEQ ID NO: 2593, wherein treated T cells were incubated with CD3 antibody to identify the portion of the treated T cells that have the TRAC gene knocked out. Absence of CD3 protein on surface of the treated T cells indicates successful knock out of the TRAC gene. FIG. 27B shows FACS analysis of T cells treated with the RNP complex and AAV6 particles containing a donor nucleotide sequence encoding CD19 CAR protein, wherein treated T cells were incubated with CD19 antibody to identify portion of the treated T cells that have successfully knocked in CD19. Presence of CD19 protein on surface of the treated T cells indicates successful knock in of CD19 into TRAC locus.

FIG. 28 illustrates results of an RNP of CasΦ.12 effector protein and a guide RNA mediated single-stranded oligodeoxynucleotides (ssODNs) integration into B2M locus and TRAC locus. For negative control, naïve T cells were treated with ssODN only.

FIG. 29 shows a schematic illustration of a study design for determining effector protein mediated GFP integration by HDR pathway in T cells.

FIGS. 30A-30F show comparisons of GFP integration into TRAC locus of T cells, wherein an effector protein was delivered to the T cells by an RNP comprising the effector protein or an mRNA encoding the effector protein. FIGS. 30A and 30D show the portion of T cells that were not expressing CD3 protein post-treatment with the RNP comprising the effector protein or the mRNA encoding the effector protein, respectively, wherein the T cells were incubated with an antibody recognizing CD3 protein. Absence of CD3 protein on T cell surface indicates that TRAC gene is successfully knocked out. FIGS. 30B and 30E show the portion of T cells that were expressing GFP protein post-treatment with the RNP comprising the effector protein or the mRNA encoding the effector protein, respectively, wherein treated cells were further transduced with AAV6 particles comprising a donor nucleotide sequence encoding the EGFP-CAR. GFP expression indicates successful integration of the donor nucleotide sequence. FIGS. 30C and 30F shows negative controls, wherein naïve T cells were treated only the AAV6 particles.

FIGS. 31A-31B shows FACS analysis 6 days post-transfection. FIG. 31A shows alternate representation of the data shown in FIGS. 30A and 30D, wherein the data illustrates the portion of T cells that do not express CD3 protein on their surface. Absence of CD3 protein on T cell surface indicates that TRAC gene is successfully knocked out. FIG. 31B shows alternate representation of the data shown in FIGS. 30B and 30E, wherein the data illustrates the portion of T cells that expresses GFP protein, which indicates successful integration of the donor nucleotide sequence encoding the EGFP-CAR. In FIGS. 31A-31B, “NT” refers to negative control data shown in FIGS. 30C and 30F, wherein naïve T cells were treated with the AAV6 particles only.

FIG. 32 shows a schematic illustration of a study design for determining effector protein mediated of promoter-less CD19-CAR into TRAC locus of T cells.

FIG. 33 shows a combined data for TRAC gene knock-out and GFP knock-in. Specifically, the portion of treated T cells that have GFP protein present, but no CD3 expression, are shown in top left corner (Q5). The portion of treated T cells that do not express either of the GFP protein and CD3 protein are shown in bottom left corner (Q8). The portion of treated T cells that expresses the CD3 protein but do not express the GFP protein are shown in bottom right corner (Q7). The portion of treated T cells that expresses both, the CD3 protein and the GFP protein, are shown in top right corner (Q6).

FIG. 34 shows exemplary results of a NALM6 cell killing assay. Specifically, the results show a portion of NALM6 cells (10,000 cells) that were killed when incubated with T cells knocked in with a donor nucleotide encoding CD19-CAR (10,000 or 50,000 cells) and a donor nucleotide encoding GFP (10,000 or 50,000 cells). The term “only T” refers to a negative control, wherein untreated T cells were incubated with NALM6 cells. “**” or “***” indicates that that the difference between two results is statistically significant.

FIG. 35 shows the portion of T cells that showed B2M gene knocked out upon treatment with an RNP complex comprising CasΦ.12 L26R effector protein, wherein the cells were incubated with B2M antibody. Absence of B2M expression on surface of the T cells indicates successful knock out of B2M gene. Cas9 was used as a positive control. NT refers to nontreated cells.

FIGS. 36A-36B show T cell memory profiles in B2M gene knocked out T cells. The profiles were analyzed based on the portions of human T cell that differentiated into stem cell memory T cell (T_{SCM}), central memory T cell (T_{CM}), effector memory T cell (T_{EM}) and terminally differentiated T cell (T_{TE}). Each column shows the portions of human T cells, in order from bottom to top, T_{CM}, T_{SCM}, T_{EM}, and T_{TE}, respectively. The analysis was performed by incubating the T cells with CD4 antihuman antibody (FIG. 36A) and CD8 anti-human antibody (FIG. 36B). Cas9 was used as a positive control. NT refers to nontreated cells, wherein the T cells were not treated with the RNP complex. T cell donor refers to reference cells.

FIG. 37 shows the portion of T cells that showed B2M gene knocked out upon transfection with 500 pmol of guide RNA and 1 μg, 2 μg, 5 μg, and 10 μg of an mRNA encoding CasΦ.12 effector protein (WT Cas Phi), CasΦ.12 L26R effector protein (L26R Cas Phi), or CasM.265466 effector protein (Cas265466). The portion of B2M gene knocked out T cells was determined by incubating with a B2M antibody. Absence of B2M protein expression on surface of the T cells indicates successful knock out of B2M gene. Cas9 was used as a positive control. NT refers to nontreated cells.

FIG. 38 shows the portion of T cells that showed B2M gene knocked out upon transfection with 500 pmol of guide RNA and 1 μg, 2 μg, 5 μg, and 10 μg of an mRNA encoding CasΦ.12 effector protein (WT Cas Phi), CasΦ.12 L26R effector protein (L26R Cas Phi), or CasM.265466 effector protein (Cas265466). The portion of B2M gene knocked out T cells was determined by determining % indel observed. Cas9 was used as a positive control. NT refers to nontreated cells.

FIGS. 39A-39D show T cell memory profiles in B2M gene knocked out T cells. The profiles were analyzed based on the portions of human T cell that differentiated into stem cell memory T cell (T_{SCM}), central memory T cell (T_{CM}), effector memory T cell (T_{EM}) and terminally differentiated T cell (T_{TE}). Each column shows the portions of human T cells, in order from bottom to top, T_{CM}, T_{SCM}, T_{EM}, and T_{TE}, respectively. In some columns, only three human T cell portions, T_{CM}, T_{SCM}, and T_{EM}, are visible. The B2M gene was knocked out by transfecting T cells with 500 pmol of guide RNA and 1 μg, 2 μg, 5 μg, and 10 μg of an mRNA encoding CasΦ.12 effector protein (WT Cas Phi), CasΦ.12 L26R effector protein (L26R Cas Phi), or CasM.265466 effector protein (Cas265466). The analysis was performed by incubating treated T cells with CD4 antihuman antibody. Cas9 was used as a positive control. NT refers to nontreated cells, wherein the T cells were not treated with the RNP complex. T cell donor refers to reference cells.

FIGS. 40A-40D show T cell memory profiles in B2M gene knocked out T cells. The profiles were analyzed based on the portions of human T cells that differentiated into stem cell memory T cell (T_{SCM}), central memory T cell (T_{CM}), effector memory T cell (T_{EM}) and terminally differentiated T cell (T_{TE}). Each column shows the portions of human T cells, in order from bottom to top, T_{CM}, T_{SCM}, T_{EM}, and T_{TE}, respectively. In some columns, only three human T cell portions, T_{CM}, T_{SCM}, and T_{EM}, are visible. The B2M gene was knocked out by transfecting T cells with 500 pmol of guide RNA and 1 μg, 2 μg, 5 μg, and 10 μg of an mRNA encoding CasΦ.12 effector protein (WT Cas Phi), CasΦ.12 L26R effector protein (L26R Cas Phi), or CasM.265466 effector protein (Cas265466). The analysis was performed by incubating treated T cells with CD8 antihuman antibody. Cas9 effector protein was used as a positive control. NT refers to nontreated cells, wherein the T cells were not treated with the RNP complex. T cell donor refers to reference cells.

FIG. 41 illustrates the nuclease activity of CasM.265466 with flexible PAM sequences, in accordance with an embodiment of the present disclosure.

FIGS. 42A-42I illustrate results of CasM.265466 mediated GFP integration in T cells. FIGS. 42A-42C show FACS analysis of T cells treated with an RNP complex of CasM.265466 effector protein and a guide RNA having a sequence of SEQ ID NO: 2488, 2489 or 2490, respectively, wherein treated T cells were incubated with CD3 antibody to identify the portion of the treated T cells that have the TRAC gene knocked out. Absence of CD3 protein on surface of the treated T cells indicates successful knock out of TRAC gene. FIGS. 42D-42F show FACS analysis of T cells treated with the RNP complex and AAV6 particles containing a donor nucleotide sequence encoding a GFP marker. FIGS. 42D-42F show the portion of treated T cells expressing GFP, which indicates successfully GFP integration into TRAC gene locus. FIGS. 42G-42I show FACS analysis of negative control, wherein naïve T cells were transduced with AAV6 particles containing a donor nucleotide sequence encoding a GFP marker.

FIGS. 43A-43C show exemplary results of NGS and FACS analysis 6 days post AAV addition. FIG. 43A shows an alternate representation of FACS analysis of FIGS. 42A-42C. FIG. 43B shows % indel observed by NGS with each guide RNA having SEQ ID NO: 2488 (TRAC KO-R11500), SEQ ID NO: 2489 (TRAC KO-R11510), or SEQ ID NO: 2490 (TRAC KO-R11524). Similarly, FIG. 43C shows an alternate representation of FACS analysis of FIGS. 42D-42F. In FIGS. 43A-43C, “NT” refers to T cells that were not treated. Similarly, in FIGS. 43A and 43C, “TRAC KO only” refers to RNP treated T cells, “TRAC KO+AAV KI” refers to RNP treated T cells that were transduced with AAV6 particles containing a donor nucleotide sequence encoding a GFP marker, and “AAV only” refers to naïve T cells that were only transduced with AAV6 particles.

FIG. 44 illustrates the effects of an arginine substitution on CasM.265466 nuclease activity for a target nucleic acid, in accordance with an embodiment of the present disclosure.

FIG. 45 illustrates the dose titration curves of CasM.265466 arginine mutants, in accordance with an embodiment of the present disclosure.

FIGS. 46A-46B show results of NGS analysis for MLH1 gene editing by CasM.265466 effector protein relative to D220R variant thereof. Specifically, FIG. 46A shows a % indel generated by the effector proteins. FIG. 46B shows a donor nucleic acid insertion in effector protein treated HEK293T cells.

FIG. 47 shows the portion of T cells that showed B2M gene knocked out upon transfection with 500 pmol of guide RNA and 1 μg, 2 μg, 5 μg, and 10 μg of an mRNA encoding CasM.265466 effector protein (WT Cas466), and CasM.265466 D220R effector protein (D220R Cas 466). The portion of B2M gene knocked out T cells were determined by determining % indel observed. NT refers to nontreated cells.

FIG. 48 shows the portion of T cells that showed TRAC gene knocked out upon transfection with 500 pmol of guide RNA and 1 μg, 2 μg, 5 μg, and 10 μg of an mRNA encoding CasM.265466 effector protein (WT Cas466), CasM.265466 D220R effector protein (D220R Cas 466), and CasΦ.12 L26R effector protein (L26R Cas Phi). The portion of TRAC gene knocked out T cells were determined by determining % indel observed. Cas9 effector protein was used as a positive control. NT refers to nontreated cells.

### DETAILED DESCRIPTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### Definitions

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term, “comprise” and its grammatical equivalents, specifies the presence of stated features, integers, steps, operations, elements, and/or components, but does not preclude the presence or addition of one or more other features, integers, steps, operations, elements, components, and/or groups thereof.

As used herein, the term, “about,” in reference to a number or range of numbers, is understood to mean the stated number and numbers +/−10% thereof, or 10% below the lower listed limit and 10% above the higher listed limit for the values listed for a range.

The terms, “% identical,” “% identity,” and “percent identity,” or grammatical equivalents thereof, as used herein, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The term, “antigen,” as used herein, refers to a compound, composition, or substance that can be specifically bound by the products of specific humoral or cellular immunity (e.g., an antibody or T-cell receptor) and induce an immune response. An antigen can be any type of molecule including, for example, proteins, haptens, simple intermediary metabolites, sugars (e.g., oligosaccharides), lipids, and hormones, as well as macromolecules such as complex carbohydrates (e.g., polysaccharides) and phospholipids. Common categories of antigens include, but are not limited to, cancer cell antigens, tumor antigens, viral antigens, bacterial antigens, fungal antigens, protozoa and other parasitic antigens, antigens involved in autoimmune disease, allergy and graft rejection, toxins, and other miscellaneous antigens.

The term, “cancer,” as used herein, refers to a disease state characterized by the presence in a subject of cells demonstrating abnormal uncontrolled replication. The term cancer can be used interchangeably with the terms “carcino-,” “onco-,” and “tumor.” Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

The terms, “chimeric antigen receptor” and “CAR,” as used herein, refer to a fused protein comprising an extracellular domain capable of binding to an antigen, a transmembrane domain derived from a polypeptide different from a polypeptide from which the extracellular domain is derived, and at least one intracellular domain. A CAR is sometimes referred to in the art as a “chimeric receptor,” a “T-body,” or a “chimeric immune receptor (CIR).” The extracellular domain capable of binding to an antigen refers to any oligopeptide or polypeptide (e.g., antibody binding domain(s)) that can bind to an antigen. The transmembrane domain refers to any oligopeptide or polypeptide known to span the cell membrane and links the extracellular domain and the signaling domain. The intracellular domain refers to any oligopeptide or polypeptide known to function as a domain that transmits a signal to cause activation or inhibition of a biological process in a cell (primary signaling domain). In some instances, the intracellular domain can include one or more costimulatory signaling domains in addition to the primary signaling domain. A CAR can also include a hinge domain that serves as a linker between the extracellular and transmembrane domains.

The term, “CAR T cell,” as used herein, refers to a T cell that has a nucleotide sequence encoding a chimeric antigen receptor (CAR).

The terms, “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The terms, “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The terms, “CRISPR RNA” and “crRNA,” as used herein, refers to type of guide nucleic acid, wherein the nucleic acid is RNA comprising a first sequence, often referred to herein as a spacer sequence, that hybridizes to a target sequence of a target nucleic acid, and a second sequence that either a) hybridizes to a portion of a tracrRNA or b) is capable of being non-covalently bound by an effector protein. In some embodiments, the crRNA is covalently linked to an additional nucleic acid (e.g., a tracrRNA) that interacts with the effector protein.

The term, “donor nucleic acid,” as used herein, refers to a nucleic acid that is incorporated into a target nucleic acid or target sequence.

The term, “effective amount,” as used herein, refers to the amount of an agent (e.g., a cell), or combined amounts of two or more agents, that is sufficient to effect a beneficial or desired result. As a non-limiting example, when administered to a subject for the treatment of a disease, an effective amount is sufficient to affect such treatment for the disease. The effective amount will vary depending on the agent(s), the beneficial or desired result, the disease and its severity, and the age, weight, etc., of the subject.

The term, “effector protein,” as used herein, refers to a protein, polypeptide, or peptide that non-covalently binds to a guide nucleic acid to form a complex that contacts a target nucleic acid, wherein at least a portion of the guide nucleic acid hybridizes to a target sequence of the target nucleic acid. A complex between an effector protein and a guide nucleic acid can include multiple effector proteins or a single effector protein. In some instances, the effector protein modifies the target nucleic acid when the complex contacts the target nucleic acid. In some instances, the effector protein does not modify the target nucleic acid, but it is fused to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout.

The term, “guide nucleic acid,” as used herein, refers to a nucleic acid comprising: a first nucleotide sequence that hybridizes to a target nucleic acid; and a second nucleotide sequence that is capable of being non-covalently bound by an effector protein. The first sequence may be referred to herein as a spacer sequence. The second sequence may be referred to herein as a repeat sequence. In some instances, the first sequence is located 5′ of the second nucleotide sequence. In some instances, the first sequence is located 3′ of the second nucleotide sequence.

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary sequence, that is capable of being non-covalently bound by an effector protein. In some instances, the handle sequence further comprises a repeat sequence. In such instances, the intermediary sequence or a combination of the intermediary sequence and the repeat sequence is capable of being non-covalently bound by an effector protein.

The term “immunologically compatible,” as used herein, refers to an agent (e.g., a cell) that is capable of being used in transfusion or grafting without rejection by the immune system of the recipient or result in the agent (e.g., a cell) attacking the recipient's normal cells or tissues (e.g., graft-vs-host disease).

The terms “indel,” “InDel,” “insertion-deletion,” and “indel mutation,” as used herein, refers to a type of genetic mutation that results from the insertion and/or deletion of nucleotides in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation.

The term, “intermediary sequence,” as used herein, in a context of a single nucleic acid system, refers to a nucleotide sequence in a handle sequence, wherein the nucleotide sequence is capable of, at least partially, being non-covalently bound to an effector protein to form a complex (e.g., an RNP complex). An intermediary sequence is not a transactivating nucleic acid in systems, methods, and compositions described herein.

The term, “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

The term, “protospacer adjacent motif (PAM),” as used herein, refers to a nucleotide sequence found in a target nucleic acid that directs an effector protein to modify the target nucleic acid at a specific location. A PAM sequence can be required for a complex having an effector protein and a guide nucleic acid to hybridize to and modify the target nucleic acid. However, a given effector protein may not require a PAM sequence being present in a target nucleic acid for the effector protein to modify the target nucleic acid.

The term, “proximity,” as used herein, refers to the state of being very near. Whether a substance, interaction, or activity is within proximity of a reference point will depend upon the context of that substance, interaction, or activity.

The term, “recombinant,” as used herein, as applied to proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA can be present 5′ or 3′ from the open reading frame, where such sequences do not interfere with manipulation or expression of the coding regions and can act to modulate production of a desired product by various mechanisms. Thus, for example, the term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. Similarly, the term “recombinant polypeptide” or “recombinant protein” refers to one which is not naturally occurring, e.g., is made by the artificial combination of two otherwise separated segments of amino sequences through human intervention. Thus, for example, a polypeptide that includes a heterologous amino acid sequence is a recombinant polypeptide.

The term, “subject,” as used herein, refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some instances, the subject is not necessarily diagnosed or suspected of being at high risk for the disease.

The term, “T cell,” as used herein, refers to a type of lymphocyte that matures in the thymus. T cells play an important role in cell-mediated immunity and are distinguished from other lymphocytes, such as B cells, by the presence of a T-cell receptor on the cell surface. A T cell includes all types of immune cells expressing CD3, including: naïve T cells (cells that have not encountered their cognate antigens), T-helper cells (CD4⁺ cells), cytotoxic T-cells (CD8⁺ cells), natural killer T-cells, T-regulatory cells (T-reg) and gamma-delta T cells. Non-limiting exemplary sources for commercially available T cell lines include the American Type Culture Collection, or ATCC, and the German Collection of Microorganisms and Cell Cultures.

The term, “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid can comprise RNA, DNA, or a combination thereof. A target nucleic acid can be single-stranded (e.g., single-stranded RNA or single-stranded DNA) or double-stranded (e.g., double-stranded DNA).

The term, “target sequence,” as used herein, when used in reference to a target nucleic acid, refers to a sequence of nucleotides found within a target nucleic acid. Such a sequence of nucleotides can, for example, hybridize to an equal length portion of a guide nucleic acid. Hybridization of the guide nucleic acid to the target sequence can bring an effector protein into contact with the target nucleic acid.

The term, “trans-activating RNA (tracrRNA),” as used herein, refers to a nucleic acid that comprises a first sequence that is capable of being non-covalently bound by an effector protein. TracrRNAs can comprise a second sequence that hybridizes to a portion of a crRNA, which may be referred to as a repeat hybridization sequence. In some embodiments, tracrRNAs are covalently linked to a crRNA.

The terms, “viral particle” and “virion,” as used herein, refer to the infective system of a virus as it exists outside of the host cell. A viral particle is typically composed of a viral genome and a protein coat called a capsid, which can be naked or enclosed in a lipoprotein envelope called the peplos. In some instances, the viral genome of a viral particle includes a viral vector. Non-limiting examples of viruses that a viral particle can be based on include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses.

The term, “viral vector,” as used herein, refers to a nucleic acid to be delivered into a host cell via a recombinantly produced viral particle. The nucleic acid can be single-stranded or double stranded, linear or circular, segmented or non-segmented. The nucleic acid can comprise DNA, RNA, or a combination thereof. Non-limiting examples of viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by viral particles may be referred to by the type of virus to deliver the viral vector (e.g., an AAV viral vector is a viral vector that is to be delivered by an adeno-associated virus particle). A viral vector referred to by the type of viral particle to deliver the viral vector can contain viral elements (e.g., nucleotide sequences) necessary for packaging of the viral vector into the virus or viral particle, replicating the virus, or other desired viral activities. A viral particle containing a viral vector can be replication competent, replication deficient or replication defective.

The terms, “beta-2 microglobulin” and “B2M,” as used herein, refer to the beta-2 microglobulin from any vertebrate source, including mammals such as primates (e.g., humans), dogs, and rodents (e.g., mice and rats), unless otherwise indicated. Beta-2-microglobulin is a serum protein found in association with the major histocompatibility complex (MHC) class I heavy chain on the surface of nearly all nucleated cells. The gene encoding human beta-2 microglobulin, referred to as B2M, contains 4 exons and spans approximately 8 kb, and is located on chromosome 15, at cytogenetic location 15q21.1. The amino acid sequence of human beta-2 microglobulin can be found at GenBank Accession No. AAA51811.1 and is provided below:

(SEQ ID NO: 1576)
MSRSVALAVLALLSLSGLEGIQRTPKIQVYSRHPAENGKSNFLNCYVSGF
HQSDIEVDLLKNGERIEKVEHSDLSFSKDWSFYLLYYTEFTPTEKDEYAC
RVNHVTLSQPKIVKWDRD.

An exemplary encoding nucleic acid sequence of human beta-2 microglobulin can be found at NCBI Reference Sequence NM_004048.4 and is provided below:

(SEQ ID NO: 1577)
attcctgaagctgacagcattcgggccgagatgtctcgctccgtggcctt
agctgtgctcgcgctactctctctttctggcctggaggctatccagcgta
ctccaaagattcaggtttactcacgtcatccagcagagaatggaaagtca
aatttcctgaattgctatgtgtctgggtttcatccatccgacattgaagt
tgacttactgaagaatggagagagaattgaaaaagtggagcattcagact
tgtctttcagcaaggactggtctttctatctcttgtactacactgaattc
acccccactgaaaaagatgagtatgcctgccgtgtgaaccatgtgacttt
gtcacagcccaagatagttaagtgggatcgagacatgtaagcagcatcat
ggaggtttgaagatgccgcatttggattggatgaattccaaattctgctt
gcttgctttttaatattgatatgcttatacacttacactttatgcacaaa
atgtagggttataataatgttaacatggacatgatcttctttataattct
actttgagtgctgtctccatgtttgatgtatctgagcaggttgctccaca
ggtagctctaggagggctggcaacttagaggtggggagcagagaattctc
ttatccaacatcaacatcttggtcagatttgaactcttcaatctcttgca
ctcaaagcttgttaagatagttaagcgtgcataagttaacttccaattta
catactctgcttagaatttgggggaaaatttagaaatataattgacagga
ttattggaaatttgttataatgaatgaaacattttgtcatataagattca
tatttacttcttatacatttgataaagtaaggcatggttgtggttaatct
ggtttatttttgttccacaagttaaataaatcataaaacttga.

The terms, “class II major histocompatibility complex transactivator” and “CIITA,” as used herein, refer to the class II major histocompatibility complex transactivator from any vertebrate source, including mammals such as primates (e.g., humans), dogs, and rodents (e.g., mice and rats), unless otherwise indicated. Class II major histocompatibility complex transactivator is protein with an acidic transcriptional activation domain, 4 LRRs (leucine-rich repeats) and a GTP binding domain. The protein is located in the nucleus and is the master regulator of MCH class II gene transcription and contributes to the transcription of MHC class I genes. The protein also uses GTP to facilitate its transport into the nucleus, and once there it uses an intrinsic acetyltransferase (AT) activity to act in a coactivator-like fashion. The gene encoding human class II major histocompatibility complex transactivator, referred to as CIITA, is located on chromosome 16, at cytogenetic location 16p13.13. The amino acid sequence of human beta-2 microglobulin can be found at GenBank Accession No. CAA52354.1 and is provided below:

(SEQ ID NO: 1578)
MRCLAPRPAGSYLSEPQGSSQCATMELGPLEGGYLELLNSDADPLCLYHF
YDQMDLAGEEEIELYSEPDTDTINCDQFSRLLCDMEGDEETREAYANIAE
LDQYVFQDSQLEGLSKDIFKHIGPDEVIGESMEMPAEVGQKSQKRPFPEE
LPADLKHWKPAEPPTVVTGSLLVGPVSDCSTLPCLPLPALFNQEPASGQM
RLEKTDQIPMPFSSSSLSCLNLPEGPIQFVPTISTLPHGLWQISEAGTGV
SSIFIYHGEVPQASQVPPPSGFTVHGLPTSPDRPGSTSPFAPSATDLPSM
PEPALTSRANMTEHKTSPTQCPAAGEVSNKLPKWPEPVEQFYRSLQDTYG
AEPAGPDGILVEVDLVQARLERSSSKSLERELATPDWAERQLAQGGLAEV
LLAAKEHRRPRETRVIAVLGKAGQGKSYWAGAVSRAWACGRLPQYDFVFS
VPCHCLNRPGDAYGLQDLLFSLGPQPLVAADEVFSHILKRPDRVLLILDA
FEELEAQDGFLHSTCGPAPAEPCSLRGLLAGLFQKKLLRGCTLLLTARPR
GRLVQSLSKADALFELSGFSMEQAQAYVMRYFESSGMTEHQDRALTLLRD
RPLLLSHSHSPTLCRAVCQLSEALLELGEDAKLPSTLTGLYVGLLGRAAL
DSPPGALAELAKLAWELGRRHQSTLQEDQFPSADVRTWAMAKGLVQHPPR
AAESELAFPSFLLQCFLGALWLALSGEIKDKELPQYLALTPRKKRPYDNW
LEGVPRFLAGLIFQPPARCLGALLGPSAAASVDRKQKVLARYLKRLQPGT
LRARQLLELLHCAHEAEEAGIWQHVVQELPGRLSFLGTRLTPPDAHVLGK
ALEAAGQDFSLDLRSTGICPSGLGSLVGLSCVTRFRAALSDTVALWESLR
QHGETKLLQAAEEKFTIEPFKAKSLKDVEDLGKLVQTQRTRSSSEDTAGE
LPAVRDLKKLEFALGPVSGPQAFPKLVRILTAFSSLQHLDLDALSENKIG
DEGVSQLSATFPQLKSLETLNLSQNNITDLGAYKLAEALPSLAASLLRLS
LYNNCICDVGAESLARVLPDMVSLRVMDVQYNKFTAAGAQQLAASLRRCP
HVETLAMWTPTIPFSVQEHLQQQDSRISLR.

An exemplary encoding nucleic acid sequence of human class II major histocompatibility complex transactivator can be found at NCBI Reference Sequence No. NM_001286402.1 and is provided below:

(SEQ ID NO: 1579)
ggttagtgatgaggctagtgatgaggctgtgtgcttctgagctgggcatccgaaggcatccttggggaagctgagggcacgagg
aggggctgccagactccgggagctgctgcctggctgggattcctacacaatgcgttgcctggctccacgccctgctgggtcctacctgtcaga
gccccaaggcagctcacagtgtgccaccatggagttggggcccctagaaggtggctacctggagcttcttaacagcgatgctgaccccctgt
gcctctaccacttctatgaccagatggacctggctggagaagaagagattgagctctactcagaacccgacacagacaccatcaactgcgac
cagttcagcaggctgttgtgtgacatggaaggtgatgaagagaccagggaggcttatgccaatatcgcggaactggaccagtatgtcttccag
gactcccagctggagggcctgagcaaggacattttcatagagcacataggaccagatgaagtgatcggtgagagtatggagatgccagcag
aagttgggcagaaaagtcagaaaagacccttcccagaggagcttccggcagacctgaagcactggaagccagctgagccccccactgtggt
gactggcagtctcctagtgggaccagtgagcgactgctccaccctgccctgcctgccactgcctgcgctgttcaaccaggagccagcctccg
gccagatgcgcctggagaaaaccgaccagattcccatgcctttctccagttcctcgttgagctgcctgaatctccctgagggacccatccagttt
gtccccaccatctccactctgccccatgggctctggcaaatctctgaggctggaacaggggtctccagtatattcatctaccatggtgaggtgcc
ccaggccagccaagtaccccctcccagtggattcactgtccacggcctcccaacatctccagaccggccaggctccaccagccccttcgctc
catcagccactgacctgcccagcatgcctgaacctgccctgacctcccgagcaaacatgacagagcacaagacgtcccccacccaatgccc
ggcagctggagaggtctccaacaagcttccaaaatggcctgagccggtggagcagttctaccgctcactgcaggacacgtatggtgccgag
cccgcaggcccggatggcatcctagtggaggtggatctggtgcaggccaggctggagaggagcagcagcaagagcctggagcgggaac
tggccaccccggactgggcagaacggcagctggcccaaggaggcctggctgaggtgctgttggctgccaaggagcaccggcggccgcgt
gagacacgagtgattgctgtgctgggcaaagctggtcagggcaagagctattgggctggggcagtgagccgggcctgggcttgtggccgg
cttccccagtacgactttgtcttctctgtcccctgccattgcttgaaccgtccgggggatgcctatggcctgcaggatctgctcttctccctgggcc
cacagccactcgtggcggccgatgaggttttcagccacatcttgaagagacctgaccgcgttctgctcatcctagacggcttcgaggagctgg
aagcgcaagatggcttcctgcacagcacgtgcggaccggcaccggcggagccctgctccctccgggggctgctggccggccttttccaga
agaagctgctccgaggttgcaccctcctcctcacagcccggccccggggccgcctggtccagagcctgagcaaggccgacgccctatttga
gctgtccggcttctccatggagcaggcccaggcatacgtgatgcgctactttgagagctcagggatgacagagcaccaagacagagccctg
acgctcctccgggaccggccacttcttctcagtcacagccacagccctactttgtgccgggcagtgtgccagctctcagaggccctgctggag
cttggggaggacgccaagctgccctccacgctcacgggactctatgtcggcctgctgggccgtgcagccctcgacagcccccccggggcc
ctggcagagctggccaagctggcctgggagctgggccgcagacatcaaagtaccctacaggaggaccagttcccatccgcagacgtgagg
acctgggcgatggccaaaggcttagtccaacacccaccgcgggccgcagagtccgagctggccttccccagcttcctcctgcaatgcttcct
gggggccctgtggctggctctgagtggcgaaatcaaggacaaggagctcccgcagtacctagcattgaccccaaggaagaagaggcccta
tgacaactggctggagggcgtgccacgctttctggctgggctgatcttccagcctcccgcccgctgcctgggagccctactcgggccatcgg
cggctgcctcggtggacaggaagcagaaggtgcttgcgaggtacctgaagcggctgcagccggggacactgcgggcgcggcagctgctg
gagctgctgcactgcgcccacgaggccgaggaggctggaatttggcagcacgtggtacaggagctccccggccgcctctcttttctgggca
cccgcctcacgcctcctgatgcacatgtactgggcaaggccttggaggcggcgggccaagacttctccctggacctccgcagcactggcatt
tgcccctctggattggggagcctcgtgggactcagctgtgtcacccgtttcagggctgccttgagcgacacggtggcgctgtgggagtccctg
cagcagcatggggagaccaagctacttcaggcagcagaggagaagttcaccatcgagcctttcaaagccaagtccctgaaggatgtggaag
acctgggaaagcttgtgcagactcagaggacgagaagttcctcggaagacacagctggggagctccctgctgttcgggacctaaagaaact
ggagtttgcgctgggccctgtctcaggcccccaggctttccccaaactggtgcggatcctcacggccttttcctccctgcagcatctggacctg
gatgcgctgagtgagaacaagatcggggacgagggtgtctcgcagctctcagccaccttcccccagctgaagtccttggaaaccctcaatct
gtcccagaacaacatcactgacctgggtgcctacaaactcgccgaggccctgccttcgctcgctgcatccctgctcaggctaagcttgtacaat
aactgcatctgcgacgtgggagccgagagcttggctcgtgtgcttccggacatggtgtccctccgggtgatggacgtccagtacaacaagttc
acggctgccggggcccagcagctcgctgccagccttcggaggtgtcctcatgtggagacgctggcgatgtggacgcccaccatcccattca
gtgtccaggaacacctgcaacaacaggattcacggatcagcctgagatgatcccagctgtgctctggacaggcatgttctctgaggacactaa
ccacgctggaccttgaactgggtacttgtggacacagctcttctccaggctgtatcccatgagcctcagcatcctggcacccggcccctgctgg
ttcagggttggcccctgcccggctgcggaatgaaccacatcttgctctgctgacagacacaggcccggctccaggctcctttagcgcccagtt
gggtggatgcctggtggcagctgcggtccacccaggagccccgaggccttctctgaaggacattgcggacagccacggccaggccagag
ggagtgacagaggcagccccattctgcctgcccaggcccctgccaccctggggagaaagtacttctttttttttatttttagacagagtctcactgt
tgcccaggctggcgtgcagtggtgcgatctgggttcactgcaacctccgcctcttgggttcaagcgattcttctgcttcagcctcccgagtagct
gggactacaggcacccaccatcatgtctggctaatttttcatttttagtagagacagggttttgccatgttggccaggctggtctcaaactcttgac
ctcaggtgatccacccacctcagcctcccaaagtgctgggattacaagcgtgagccactgcaccgggccacagagaaagtacttctccaccc
tgctctccgaccagacaccttgacagggcacaccgggcactcagaagacactgatgggcaacccccagcctgctaattccccagattgcaac
aggctgggcttcagtggcagctgcttttgtctatgggactcaatgcactgacattgttggccaaagccaaagctaggcctggccagatgcacca
gcccttagcagggaaacagctaatgggacactaatggggcggtgagaggggaacagactggaagcacagcttcatttcctgtgtcttttttcac
tacattataaatgtctctttaatgtcacaggcaggtccagggtttgagttcataccctgttaccattttggggtacccactgctctggttatctaatatg
taacaagccaccccaaatcatagtggcttaaaacaacactcacattta.

The terms, “T-cell receptor alpha-constant” and “TRAC,” as used herein, refer to the T-cell receptor alpha-constant from any vertebrate source, including mammals such as primates (e.g., humans), dogs, and rodents (e.g., mice and rats), unless otherwise indicated. T-cell receptor alpha-constant is the C-terminal portion of the T-cell receptor alpha chain, which is formed when 1 of at least 70 variable (V) genes, which encode the N-terminal antigen recognition domain, rearranges to 1 of 61 joining (J) gene segments to create a functional V region exon that is transcribed and spliced to the constant region gene (TRAC) segment. The gene encoding human T-cell receptor alpha-constant, referred to as TRAC, is located on chromosome 14, at cytogenetic location 14q11.2. The amino acid sequence of T-cell receptor alpha-constant can be found at UniProtKB/Swiss-Prot No. P01848.2 and is provided below:

(SEQ ID NO: 1580)
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVE
KSFETDTNLNFQNLSVIGFRILLLKVAGFNLLMTLRLWSS.

An exemplary encoding nucleic acid sequence of human T-cell receptor alpha-constant can be found at Ensembl No. ENST00000611116.2 and is provided below:

(SEQ ID NO: 1581)
atatccagaaccctgaccctgccgtgtaccagctgagagactctaaatcc
agtgacaagtctgtctgcctattcaccgattttgattctcaaacaaatgt
gtcacaaagtaaggattctgatgtgtatatcacagacaaaactgtgctag
acatgaggtctatggacttcaagagcaacagtgctgtggcctggagcaac
aaatctgactttgcatgtgcaaacgccttcaacaacagcattattccaga
agacaccttcttccccagcccagaaagttcctgtgatgtcaagctggtcg
agaaaagctttgaaacagatacgaacctaaactttcaaaacctgtcagtg
attgggttccgaatcctcctcctgaaagtggccgggtttaatctgctcat
gacgctgcggctgtggtccagctga.

Disclosed herein are non-naturally occurring compositions (e.g., viral vector, viral particle, CAR T cell, population of CAR T cells), kits, and systems comprising an effector protein (e.g., an engineered effector protein) and an engineered guide nucleic acid, which may simply be referred to herein as a guide nucleic acid. In general, an engineered effector protein and an engineered guide nucleic acid refer to an effector protein and a guide nucleic acid, respectively, that are not found in nature. In some embodiments, the compositions, kits, and systems comprise at least one non-naturally occurring component. For example, compositions, kits, and systems can comprise a guide nucleic acid, wherein the sequence of the guide nucleic acid is different or modified from that of a naturally occurring guide nucleic acid. In some embodiments, compositions, kits and systems comprise at least two components that do not naturally occur together. For example, compositions, kits and systems can comprise a guide nucleic acid comprising a repeat region and a spacer region which do not naturally occur together. Also, by way of example, compositions, kits, and systems can comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

There are a number of ways in which the compositions (e.g., viral vector, viral particle, CAR T cell, population of CART cells), kits, and systems described herein can be non-naturally occurring based on the guide nucleic acid. In some embodiments, the guide nucleic acid comprises a non-natural nucleotide sequence. In some embodiments, the non-natural sequence is a nucleotide sequence that is not found in nature. The non-natural sequence can comprise a portion of a naturally occurring sequence, wherein the portion of the naturally-occurring sequence is not present in nature, absent the remainder of the naturally-occurring sequence. In some embodiments, the guide nucleic acid comprises two naturally occurring sequences arranged in an order or proximity that is not observed in nature. In some embodiments, compositions, kits, and systems comprise a ribonucleotide complex comprising an effector protein and a guide nucleic acid that do not occur together in nature. Engineered guide nucleic acids can comprise a first sequence and a second sequence that do not occur naturally together. For example, an engineered guide nucleic acid can comprise a sequence of a naturally occurring repeat region and a spacer region that is complementary to a naturally-occurring eukaryotic sequence. The engineered guide nucleic acid can comprise a sequence of a repeat region that occurs naturally in an organism and a spacer region that does not occur naturally in that organism. An engineered guide nucleic acid can comprise a first sequence that occurs in a first organism and a second sequence that occurs in a second organism, wherein the first organism and the second organism are different. The guide nucleic acid can comprise a third sequence located at a 3′ or 5′ end of the guide nucleic acid, or between the first and second sequences of the guide nucleic acid. For example, an engineered guide nucleic acid can comprise a naturally occurring crRNA and tracrRNA sequence coupled by a linker sequence.

Similarly, there are a number of ways in which the compositions (e.g., viral vector, viral particle, CAR T cell, population of CAR T cells), kits, and systems described herein can be non-naturally occurring based on the effector protein. In some embodiments, compositions, kits, and systems described herein comprise an engineered effector protein that is similar to a naturally occurring effector protein. The engineered effector protein can lack a portion of the naturally occurring effector protein. The effector protein can comprise a mutation relative to the naturally occurring effector protein, wherein the mutation is not found in nature. The effector protein can also comprise at least one additional amino acid relative to the naturally occurring effector protein. For example, the effector protein can comprise an addition of a nuclear localization signal relative to the natural occurring effector protein. In certain embodiments, the nucleotide sequence encoding the effector protein is codon optimized (e.g., for expression in a eukaryotic cell) relative to the naturally occurring sequence.

### Vectors and Multiplexed Expression Vectors

Compositions, systems, and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises effector proteins(s), guide nucleic acid(s), target nucleic acid(s), and donor nucleic acid(s). In some embodiments, the component comprises a nucleic acid encoding an effector protein, a donor nucleic acid, and a guide nucleic acid or a nucleic acid encoding the guide nucleic acid. In some embodiments, a vector may be part of a vector system. The vector system may comprise a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., an effector protein, a guide nucleic acid, and/or a target nucleic acid) are each encoded by different vectors of the system.

In some embodiments, a vector comprises a nucleotide sequence encoding one or more effector proteins as described herein. In some embodiments, the one or more effector proteins comprise at least two effector proteins. In some embodiments, the at least two effector protein are the same. In some embodiments, the at least two effector proteins are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more effector proteins.

In some embodiments, a vector may encode one or more of any system components, including but not limited to effector proteins, guide nucleic acids, donor nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system components. For example, a vector may encode two or more guide nucleic acids, wherein each guide nucleic acid comprises a different sequence. A vector may encode an effector protein and a guide nucleic acid. A vector may encode an effector protein, a guide nucleic acid, and a donor nucleic acid.

In some embodiments, a vector comprises one or more guide nucleic acids, or a nucleotide sequence encoding the one or more guide nucleic acids. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector comprises one or more donor nucleic acids. In some embodiments, the one or more donor nucleic acids comprise at least two donor nucleic acids. In some embodiments, the at least two donor nucleic acids are the same. In some embodiments, the at least two donor nucleic acids are different from each other. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor nucleic acids.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter —a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence. The promoter can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or the effector protein.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, EF1a, 7SK, RPBSA, hPGK, EFS, SV40, PGK1, Ube, human beta actin promoter, CAG, MND, TRE, UAS, Ac5, Polyhedrin, CaMKIIa, GAL1-10, H1, TEF1, GDS, ADH1, CaMV35S, HSV TK, Ubi, U6, MNDU3, and MSCV. In some embodiments, the promoter for the guide nucleic acid is a U6 promoter, having a length of about 249 linked nucleotides. In some embodiments, the promoter for the Cas effector is an EFS promoter, having a length of about 231 linked nucleotides.

In some embodiments, the promoter for expressing effector protein is a ubiquitous promoter. In some embodiments, the ubiquitous promoter comprises MND or CAG promoter sequence. In some embodiments, the promoter is a tissue-specific promoter that has activity in only certain cell types. In some embodiments, the cell type is a T cell. Non-limiting examples of promoters particularly suitable for T cell expression include a EF-1 promoter, an RPBSA promoter, a hPGK promoter, and a CMV promoter, as described further in Rad et al., (2020), PLoS ONE, 15(7):e0232915. In some embodiments, the promoter is a constitutive promoter. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44.

In some embodiments, the promoters are prokaryotic promoters (e.g., drive expression of a gene in a prokaryotic cell). In some embodiments, the promoters are eukaryotic promoters, (e.g. drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

In some embodiments, vectors described herein include elements for abrogating allogeneic immune reactions of T cells when transfused or grafted into a subject, while simultaneously directing the immune activity of the T cells to a specific antigen (e.g., a cancer specific antigen expressed by a cancer cell) through introduction of a donor nucleic acid encoding a chimeric antigen receptor (CAR). Accordingly, vectors provided herein comprises a first nucleotide sequence that encodes an effector protein, a second nucleotide sequence that produces a guide nucleic acid for targeting the effector protein to the gene encoding T-cell receptor alpha-constant (TRAC gene), a third nucleotide sequence that produces a guide nucleic acid for targeting the effector protein to the gene encoding beta-2 microglobulin (B2M gene), a fourth nucleotide sequence that produces a guide nucleic acid for targeting the effector protein to the gene encoding human class II major histocompatibility complex transactivator (CIITA gene), and/or a fifth nucleotide sequence that includes a donor nucleic acid encoding a CAR and a nucleotide sequence that directs integration of the donor nucleic acid into the TRAC gene.

In some cases, the second nucleotide sequence when transcribed and/or cleaved by the effector protein, produces a guide nucleic acid. In some embodiments, the guide nucleic acid comprises a sequence that is at least 80%, at least 85%, at least 90%, at least 95%, at least 96%, at least 97%, at least 98%, at least 99%, or 100% identical or complementary to an equal length portion of a target sequence of a gene encoding the human T-cell receptor alpha-constant (TRAC gene), the human beta-2 microglobulin (B2M gene), or the human class II major histocompatibility complex transactivator (CIITA gene). In some embodiments, the guide nucleic acid comprises a nucleotide sequence that the effector protein binds. In some embodiments, the effector protein comprises a sequence with at least about: 80%, 85%, 90%, 95%, 96%, 97%, 98%, or 99% sequence homology to any one of the amino acid sequences recited in TABLE 1. In some embodiments, the effector protein comprises a sequence with at least about: 80%, 85%, 90%, 95%, 96%, 97%, 98%, or 99% sequence homology to SEQ ID NO: 2435. In some embodiments, the guide nucleic acid comprises a sequence with at least about: 80%, 85%, 90%, 95%, 96%, 97%, 98%, or 99% sequence identity to any one of the sequences recited in TABLE 5, TABLE 5.1, TABLE 9, TABLE 9.1, TABLE 10, TABLE 10.1, TABLE 14, and TABLE 14.1. In some embodiments, the guide nucleic acid comprises any one of the sequences recited in TABLE 5, TABLE 5.1, TABLE 9, TABLE 9.1, TABLE 10, TABLE 10.1, TABLE 14, and TABLE 14.1. In some embodiments, the guide nucleic acid comprises a sequence with at least about: 80%, 85%, 90%, 95%, 96%, 97%, 98%, or 99% sequence identity to any one of the sequences recited in TABLE 6, TABLE 6.1, TABLE 11, TABLE 11.1, TABLE 12, TABLE 12.1, TABLE 15, and TABLE 15.1. In some embodiments, the guide nucleic acid comprises any one of the sequences recited in TABLE 6, TABLE 6.1, TABLE 11, TABLE 11.1, TABLE 12, TABLE 12.1, TABLE 15, and TABLE 15.1. In some embodiments, the guide nucleic acid comprises a sequence with at least about: 80%, 85%, 90%, 95%, 96%, 97%, 98%, or 99% sequence identity to any one of the sequences recited in TABLE 7, TABLE 7.1, TABLE 8, TABLE 13, and TABLE 16. In some embodiments, the guide nucleic acid comprises any one of the sequences recited in TABLE 7, TABLE 7.1, TABLE 8, TABLE 13, and TABLE 16.

Alternatively, or in addition to targeting the T-cell receptor alpha-constant (TRAC gene) as described herein, in some embodiments, guide nucleic acids can be designed for targeting one or more of the human T-cell receptor f chain variable regions similar to the TRAC gene. Accordingly, in some embodiments, the guide nucleic is capable of being bound by an effector protein having any one of the amino acid sequence recited in TABLE 1, wherein the guide nucleic acid comprises a spacer sequence that is at least 80%, at least 85%, at least 90%, at least 95%, at least 96%, at least 97%, at least 98%, at least 99%, or 100% identical or complementary to an equal length portion of a target sequence of a gene encoding any one of the thirty known human T-cell receptor R chain variable regions. In some embodiments, the guide nucleic acid comprises a nucleotide sequence that is at least 80%, at least 85%, at least 90%, at least 95%, at least 98% or 100% identical to a sequence recited in any one of TABLES 2-4. Moreover, in such embodiments, the effector protein comprises a sequence with at least about: 80%, 85%, 90%, 95%, 96%, 97%, 98%, or 99% sequence homology to any one of the amino acid sequences recited in TABLE 1. In some embodiments, vectors may comprise a first nucleotide sequence that encodes an effector protein, a second nucleotide sequence that produces a guide nucleic acid for targeting the effector protein to the gene encoding T-cell receptor R chain variable region, a third nucleotide sequence that produces a guide nucleic acid for targeting the effector protein to the gene encoding beta-2 microglobulin (B2M gene), a fourth nucleotide sequence that produces a guide nucleic acid for targeting the effector protein to the gene encoding human class II major histocompatibility complex transactivator (CIITA gene), and/or a fifth nucleotide sequence that includes a donor nucleic acid encoding a CAR and a nucleotide sequence that directs integration of the donor nucleic acid into the T-cell receptor R chain variable regions.

Alternatively or in addition to targeting the B2M gene and CIITA gene as described herein, in some embodiments, guide nucleic acids can be designed for targeting a gene encoding human NOD-like receptor family CARD domain containing 5 (NLRC5 gene). Accordingly, in some embodiments, vectors may comprise: (1) a first nucleotide sequence that encodes an effector protein; (2) a second nucleotide sequence that produces a guide nucleic acid for targeting the effector protein to the gene targeting T-cell receptor (TRAC gene or a gene encoding R chain variable region); (3) at least two of the following three nucleotide sequences: (a) a third nucleotide sequence that produces a guide nucleic acid for targeting the effector protein to the gene encoding B2M gene, (b) a fourth nucleotide sequence that produces a guide nucleic acid for targeting the effector protein to CIITA gene, and (c) a fifth nucleotide sequence that produces a guide nucleic acid for targeting the effector protein to NLRC5 gene; and/or a sixth nucleotide sequence that includes a donor nucleic acid encoding a CAR and a nucleotide sequence that directs integration of the donor nucleic acid into the T-cell receptor.

Also provided herein are T-cells comprising the vector described herein. Also provided herein are NK-cells comprising the vector described herein. In some embodiments, the T-cells and/or NK-cells having the one or more genes located on one of two alleles that are being targeted as described herein are independently modified. Accordingly, in some embodiments, the T-cells and/or NK-cells comprise a modification of one allele for one or more genes described herein. In some embodiments, the T-cells and/or NK-cells comprise a modification of both alleles for the one or more gene described herein. In some embodiments, the T-cells or NK-cells comprise a modification of at least one of the two alleles of the genes being targeted, wherein the one or more genes being targeted is selected from T-cell receptor (TRAC gene or a gene encoding f chain variable region), B2M gene, CIITA gene, and NLRC5 gene. In some embodiments, the T-cells or NK-cells comprise a modification of both alleles for the one or more gens being targeted, wherein the one or more genes being targeted is selected from T-cell receptor (TRAC gene or a gene encoding f chain variable region), B2M gene, CIITA gene, and NLRC5 gene.

Also provided herein are methods of producing a population of immunologically compatible chimeric antigen receptor (CAR) T cells comprising: contacting ex vivo a population of T cells with a viral vector described herein, for a sufficient period of time to allow for viral transduction of T cells contained in the population; and culturing the population of T cells for sufficient period of time for indels to occur in the TRAC gene, the B2M gene or the CIITA gene, thereby producing the population of immunologically compatible CAR T cells.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid editing, and/or treatment as described herein. In some embodiments, a vector is administered in a single vehicle, such as a single expression vector. In some embodiments, at least two of the three components, a nucleic acid encoding one or more effector proteins, one or more donor nucleic acids, and one or more guide nucleic acids or a nucleic acid encoding the one or more guide nucleic acid, are provided in the single expression vector. In some embodiments, components, such as a guide nucleic acid and an effector protein, are encoded by the same vector. In some embodiments, an effector protein (or a nucleic acid encoding same) and/or an engineered guide nucleic acid (or a nucleic acid that, when transcribed, produces same) are not co-administered with donor nucleic acid in a single vehicle. In some embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more effector proteins, one or more donor nucleic acids, and one or more guide nucleic acids or a nucleic acid encoding the one or more guide nucleic acids, or a variant thereof is provided in a different vector. In some embodiments, the nucleic acid encoding the effector protein, and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkami et al., (2018) Nucleic Acid Therapeutics, 28(3):146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3, N5-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding the effector protein, and/or a donor nucleic acid. In some embodiments, the inner core is a hydrophobic core. In some embodiments, the one or more of a guide nucleic acid, the one or more nucleic acid encoding the one or more guide nucleic acid, one or more nucleic acid encoding one or more effector protein, and/or the one or more donor nucleic acid forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

### Viral Vectors

Disclosed herein, in some aspects, are viral vectors that include elements for abrogating allogeneic immune reactions of T cells when transfused or grafted into a subject, while simultaneously directing the immune activity of the T cells to a specific antigen (e.g., a cancer specific antigen expressed by a cancer cell) through introduction of a donor nucleic acid encoding a chimeric antigen receptor (CAR). Accordingly, viral vectors provided herein include nucleotide sequences that provide certain features: 1) a nucleotide sequence that encodes an effector protein; 2) a nucleotide sequence that produces a guide nucleic acid for targeting the effector protein to the gene encoding T-cell receptor alpha-constant (TRAC gene); 3) a nucleotide sequence that produces a guide nucleic acid for targeting the effector protein to the gene encoding beta-2 microglobulin (B2M gene); 4) a nucleotide sequence that produces a guide nucleic acid for targeting the effector protein to the gene encoding human class II major histocompatibility complex transactivator (CIITA gene); and/or 5) a nucleotide sequence that includes a donor nucleic acid encoding a CAR and a nucleotide sequence that directs integration of the donor nucleic acid into the TRAC gene.

In some embodiments, provided herein is a viral vector comprising a nucleotide sequence that encodes an effector protein as described herein. In some embodiments, provided herein is a viral vector comprising a nucleotide sequence that produces a guide nucleic acid, as described herein, for targeting the effector protein to a specific gene (e.g., TRAC gene, B2M gene and/or CIITA gene). In some embodiments, provided herein is a viral vector comprising a nucleotide sequence that comprises a donor nucleic acid and one or more nucleotide sequences for directing its integration into the TRAC gene, wherein the donor nucleic acid encodes a CAR.

Accordingly, in some embodiments, provided herein is a viral vector comprising a first nucleotide sequence that encodes an effector protein as described herein, a second nucleotide sequence that produces a first guide nucleic acid for targeting the effector protein to the TRAC gene as described herein, a third nucleotide sequence that produces a second guide nucleic acid for targeting the effector protein to the B2M gene as described herein, a fourth nucleotide sequence that produces a third guide nucleic acid for targeting the effector protein to the CIITA gene as described herein, and a fifth nucleotide sequence that comprises a donor nucleic acid encoding a CAR and one or more nucleotide sequences for directing integration of the donor nucleic acid into the TRAC gene as described herein.

In some embodiments, provided herein are viral vectors comprising: a nucleotide sequence that encodes an effector protein and a second nucleotide sequence. In some embodiments, the viral vector is an scAAV vector. In some embodiments, a plasmid encoding the scAAV vector comprises a nucleotide sequence that encodes an effector protein with at least about: 90%, 95%, 96%, 97%, 98%, or 99% sequence identity to any one of the sequences recited in TABLE 1. In some embodiments, a plasmid encoding the scAAV vector comprises a nucleotide sequence that encodes an effector protein having the amino acid sequence of any one of the sequences recited in TABLE 1. In some embodiments, a plasmid encoding the scAAV vector comprises a nucleotide sequence encoding an effector protein with at least about: 90%, 95%, 96%, 97%, 98%, or 99% sequence identity to SEQ ID NO: 2435. In some embodiments, a plasmid encoding the scAAV vector comprises a nucleotide sequence encoding an effector protein having the amino acid sequence of SEQ ID NO: 2435. Also provided herein are T-cells comprising the viral vector. Also provided herein are NK-cells comprising the viral vector.

### Delivery of Viral Vectors

In some embodiments, the viral vector comprises a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector provided herein can be derived from or based on any such virus. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein may be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

Often the viral vectors provided herein are an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV 11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

Generally, an AAV vector has two inverted terminal repeats (ITRs). According, in some embodiments, the viral vector provided herein comprises two inverted terminal repeats of AAV. Typically, the length of each ITR is about 145 bp.

The DNA sequence in between the ITRs of an AAV vector provided herein may be referred to herein as the sequence encoding the genome editing tools. These genome editing tools can include, but are not limited to, an effector protein, effector protein modifications (e.g., nuclear localization signal (NLS), polyA tail), guide nucleic acid(s), respective promoter(s), and a donor nucleic acid, or combinations thereof. Accordingly, in some embodiments, a viral vector provided herein comprises at least one promoter that drives expression of the effector protein and at least one promoter that results in the transcription of nucleotides sequences that, when transcribed and/or cleaved by the effector protein, produce the guide nucleic acid for targeting the effector protein to the TRAC gene, the guide nucleic acid for targeting the effector protein to the B2M gene, the guide nucleic acid for targeting the effector protein to the CIITA gene, or a combination thereof. In some embodiments, a viral vector provided herein comprises a single promoter for producing a single RNA transcript containing two or more guide nucleic acids contained in the sequence encoding or producing the genome editing tools. For example, in some embodiments, a viral vector provided herein comprises a promoter that drives transcription of the nucleotide sequences that produce the guide nucleic acid for targeting the effector protein to the TRAC gene, the guide nucleic acid for targeting the effector protein to the B2M gene, and the guide nucleic acid for targeting the effector protein to the CIITA gene as a single RNA transcript. In such a viral vector, the sequence encoding the genome editing tools can further comprise a second promoter that drives expression of the effector protein. In some embodiments, a viral vector provided herein comprises a separate promoter for producing each of the guide nucleic acids contained in the sequence encoding the genome editing tools. Accordingly, in some embodiments, the viral vector provided herein comprises a first promoter that drives transcription of the nucleotide sequence that produces the guide nucleic acid for targeting the effector protein to the TRAC gene, a second promoter that drives transcription of the nucleotide sequence that produces the guide nucleic acid for targeting the effector protein to the B2M gene, and a third promoter that drives transcription of the nucleotide sequence that produces the guide nucleic acid for targeting the effector protein to the CIITA gene. In such a viral vector, the sequence encoding the genome editing tools can further comprise a fourth promoter that drives expression of the effector protein. In some embodiments, a viral vector provided herein comprises a promoter for producing two of the guide nucleic acids and a separate promoter for producing a third guide nucleic acid contained in the sequence encoding the genome editing tools. Accordingly, in some embodiments, the viral vector provided herein comprises a first promoter that drives transcription of the nucleotide sequence that produces the guide nucleic acid for targeting the effector protein to the TRAC gene and the guide nucleic acid for targeting the effector protein to the B2M gene, and a second promoter that drives transcription of the nucleotide sequence that produces the guide nucleic acid for targeting the effector protein to the CIITA gene. In some embodiments, the viral vector provided herein comprises a first promoter that drives transcription of the nucleotide sequence that produces the guide nucleic acid for targeting the effector protein to the TRAC gene and the guide nucleic acid for targeting the effector protein to the CIITA gene, and a second promoter that drives transcription of the nucleotide sequence that produces the guide nucleic acid for targeting the effector protein to the B2M gene. In some embodiments, the viral vector provided herein comprises a first promoter that drives transcription of the nucleotide sequence that produces the guide nucleic acid for targeting the effector protein to the B2M gene and the guide nucleic acid for targeting the effector protein to the CIITA gene, and a second promoter that drives transcription of the nucleotide sequence that produces the guide nucleic acid for targeting the effector protein to the TRAC gene.

In general, viral vectors provided herein comprise at least one promotor or a combination of promoters driving expression or transcription of one or more genome editing tools described herein. In some embodiments, the length of the promoter is less than about 500, less than about 400, or less than about 300 linked nucleotides. In some embodiments, the length of the promoter is at least 100 linked nucleotides.

In some embodiments, the length of the sequence encoding the genome editing tools (also referred to as the cloning capacity) between the ITRs is about 4 kb to about 5 kb. In some embodiments, the length of the sequence encoding the genome editing tools is about 4.2 kb to about 4.8 kb. In some embodiments, the length of the sequence encoding the genome editing tools is about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, about 3.3 kb, about 3.4 kb, about 3.5 kb, about 3.6 kb, about 3.7 kb, about 3.8 kb, about 3.9 kb, about 4.0 kb, about 4.1kb, about 4.2 kb, about 4.3 kb, about 4.4 kb, about 4.5 kb, about 4.6 kb, about 4.7 kb, about 4.8 kb, about 4.9 kb, or about 5 kb.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. In some embodiments, the length of the sequence encoding the genome editing tools of an scAAV vector is about 2kb, about 2.1 kb, about 2.2 kb, about 2.3 kb, about 2.4 kb, about 2.5 kb, about 2.6 kb, about 2.7 kb, or about 2.8 kb. The scAAV vector can comprise nucleotide sequences encoding an effector protein, providing guide nucleic acids described herein, and a donor nucleic acid described herein.

In some embodiments, the AAV vector provided herein is a self-inactivating AAV vector. A self-inactivating AAV vector provided herein comprises guide nucleic acids described herein, wherein the guide nucleic acids comprises a region that is complementary to the region of the AAV vector encoding the effector protein described herein. In some embodiments, the AAV vector comprises guide nucleic acids described herein that comprise a region that is complementary to sequences near the 5′ and 3′ ends of the region of the AAV vector encoding the effector protein, thereby allowing for the region of the AAV vector encoding the effector protein to be excised. Thus, the effector protein can control expression of itself. In some embodiments, the self-inactivating AAV vector limits the duration of expression of the effector protein, thereby limiting off-target effector protein activity and enabling safe genome editing. In some embodiments, the self-inactivating AAV vector is a self-inactivating scAAV vector.

In some embodiments, the plasmid encoding the scAAV vector provided herein comprises a nucleotide sequence encoding an effector protein having at least about 80%, 85%, 90%, 95%, 96%, 97%, 98%, 99% or 100% sequence identity to any one of the sequences recited in TABLE 1. In some embodiments, the plasmid encoding the scAAV vector provided herein comprises a nucleotide sequence encoding an effector protein having at least about 80%, 85%, 90%, 95%, 96%, 97%, 98%, or 99% sequence identity to SEQ ID NO: 2435. In some cases, the plasmid encoding the scAAV vector provided herein comprises a nucleotide sequence encoding an effector protein having the amino acid sequence of SEQ ID NO: 2435.

In some embodiments, an AAV vector provided herein comprises a modification, such as an insertion, deletion, chemical alteration, or synthetic modification, relative to a wild-type AAV vector. In some embodiments, the modification is in a protein coding region or a non-coding region of an AAV vector. In some embodiments, a modification improves the protein expression activity of the AAV vector. In some embodiments, an AAV vector provided herein is chimeric. In some embodiments, inverted terminal repeats of an AAV vector comprise a 5′ inverted terminal repeat, a 3′ inverted terminal repeat, and a mutated inverted terminal repeat. In some embodiments, a mutated inverted terminal repeat lacks a terminal resolution site. In some embodiments, an AAV vector provided herein comprises a modification in a capsid (CAP) or replication (REP) protein. In some embodiments, an AAV vector provided herein comprises any combination of REP, CAP, and ITR sequences from different AAV serotypes. In some embodiments, an AAV vector comprises a genome comprising a replication gene and inverted terminal repeats from a first AAV serotype and a capsid protein from a second AAV serotype. In some embodiments, an AAV vector comprises a genome consisting of a sequence encoding the genome editing tools described herein and inverted terminal repeats from an AAV, with no other AAV genes (e.g., genes encoding REP proteins or genes encoding CAP proteins).

In some embodiments, an AAV vector provided herein comprises a sequence encoding the genome editing tools that allows for the AAV vector to be packaged into a viral particle. Accordingly, in some embodiments, the sequence encoding the genome editing tools comprises or consists essentially of a nucleotide sequence encoding an effector protein, nucleotide sequences that produce guide nucleic acids for targeting the effector protein to the TRAC gene, the B2M gene and the CIITA gene, a first promoter driving the expression of the effector protein, one, two or three promoters driving expression of the guide nucleic acids, and a donor nucleic acid, wherein the effector protein is less than about 600 amino acids in length or a length as described herein, the nucleotide sequences producing the guide nucleic acids total about 100 to about 300 nucleotides in length, and wherein nucleotide sequence that comprises the donor nucleic acid is about 500 nucleotides to about 2,500 nucleotides in length.

### Producing AAV Delivery Vectors

In some embodiments, methods of producing AAV delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging an effector encoding nucleic acid into the AAV particle, thereby generating an AAV delivery vector. In some embodiments, promoters, stuffer sequences, and any combination thereof may be packaged in the AAV vector. In some examples, the AAV vector may package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

### Viral Particles

Disclosed herein, in some aspects, are viral particles comprising a viral vector described herein. Such viral particles are suitable for ex vivo transduction of a target cell as described herein (e.g., a T cell). Accordingly, in some embodiments, viral particles described herein are derived from a retrovirus, an adenovirus, an arenavirus, an alphavirus, an AAV, a baculovirus, a vaccinia virus, a herpes simplex virus or a poxvirus. Such viral particles provide the infective system of the virus from which it was derived in order to facilitate delivery of the viral vector into the target cell described herein.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof. In some embodiments, the AAV serotype is AAV-DJ. AAV-DJ is a synthetic serotype with a chimeric capsid of AAV-2, 8 and 9 as further described by Grimm et al. (2008) J. Virol., 82(12):5887-911. In some embodiments, the AAV serotype is a AAV X-Vivo (AAV-XV) serotype, which is a combination of the VP1 unique (VP1u) and VP1/2-common region sequences of AAV6 with those from divergent AAV serotypes AAV4, AAV5, AAV 11, and AAV12 to create chimeric AAV6 vectors, as further described by Viney et al., (2021), J. Virol., 95(7):e02023-20, which is incorporated by reference in its entirety. Such AAV-XV particles show enhanced transduction of human primary T cells, and superior genomic integration of DNA sequences by AAV alone or in combination with CRISPR gene editing. Accordingly, in some embodiments, the viral particle described herein is an AAV-XV derived from chimeras of AAV12 VP1/2 sequences and the VP3 sequence of AAV6.

In some embodiments, an AAV particle provided herein is engineered or modified. In some embodiments, a modification comprises a deletion, insertion, mutation, substitution, or a combination thereof of the capsid protein, the rep protein, an ITR sequence, or other components of an AAV. In some embodiments, modifications to the AAV genome and/or the capsids/rep proteins can be designed to facilitate more efficient or more specific transduction of a cell described herein (e.g., T cell). In general, an AAV undergoes several steps prior to achieving gene expression: 1) binding or attachment to cellular surface receptors, 2) endocytosis, 3) trafficking to the nucleus, 4) uncoating of the virus to release the genome, and 5) conversion of the genome from single-stranded to double-stranded DNA as a template for transcription in the nucleus. In some embodiments, the cumulative efficiency with which an AAV can successfully execute each individual step can determine the overall transduction efficiency. In some embodiments, modifications of AAV can improve or modify the rate limiting steps in AAV transduction including the absence or low abundance of required cellular surface receptors for viral attachment and internalization, inefficient endosomal escape leading to lysosomal degradation, slow conversion of single-stranded to double-stranded DNA template, or a combination thereof.

In some embodiments, a viral particle described herein comprises an AAV viral capsid modified relative to a naturally occurring AAV viral capsid. In some embodiments, modifying an AAV viral capsid comprises modifying a combination of capsid components. In some embodiments, a mutated AAV virus particle comprises a mutation in at least one capsid protein. In some embodiments, the mutation is in VP1 and VP2, in VP1 and VP3, in VP2 and VP3, or in VP1, VP2, and VP3. In some embodiments, a VP is eliminated. A mutation can occur at any of AAV capsid positions described thereof and can include any number of mutations. In some embodiments, a mutation is from one amino acid to another amino acid. A mutation can comprise modifying an amino acid to any permutation of the canonical amino acids (e.g., relative to a wildtype capsid protein). Any of the following amino acid modifications can be made at any of VP1, VP2, and VP3: A to R, A to N, A to D, A to C, A to Q, A to E, A to G, A to H, A to I, A to L, A to K, A to M, A to F, A to P, A to S, A to T, A to W, A to Y, A to V, R to N, R to D, R to C, R to Q, R to E, R to G, R to H, R to I, R to L, R to K, R to M, R to F, R to P, R to S, R to T, R to W, R to Y, R to V, N to D, N to C, N to Q, N to E, N to G, N to H, N to I, N to L, N to K, N to M, N to F, N to P, N to S, N to T, N to W, N to Y, N to V, D to C, D to Q, D to E, D to G, D to H, D to I, D to L, D to K, D to M, D to F, D to P, D to S, D to T, D to W, D to Y, D to V, C to Q, C to E, C to G, C to H, C to I, C to L, C to K, C to M, C to F, C to P, C to S, C to T, C to W, C to Y, C to V, Q to E, Q to G, Q to H, Q to I, Q to L, Q to K, Q to M, Q to F, Q to P, Q to S, Q to T, Q to W, Q to Y, Q to V, E to G, E to H, E to I, E to L, E to K, E to M, E to F, E to P, E to S, E to T, E to W, E to Y, E to V, G to H, G to I, G to L, G to K, G to M, G to F, G to P, G to S, G to T, G to W, G to Y, G to V, H to I, H to L, H to K, H to M, H to F, H to P, H to S, H to T, H to W, H to Y, H to V, I to L, I to K, I to M, I to F, I to P, I to S, I to T, I to W, I to Y, I to V, L to K, L to M, L to F, L to P, L to S, L to T, L to W, L to Y, L to V, K to M, K to F, K to P, K to S, K to T, K to W, K to Y, K to V, M to F, M to P, M to S, M to T, M to W, M to Y, M to V, F to P, F to S, F to T, F to W, F to Y, F to V, P to S, P to T, P to W, P to Y, P to V, S to T, S to W, S to Y, S to V, T to W, T to Y, T to V, W to Y, W to V, Y to V, and any of the previously described mutations in reverse.

In some embodiments, a viral particle provided herein comprises a chimeric capsid. In some embodiments, a chimeric capsid comprises an insertion of a foreign protein sequence into the open reading frame of the capsid gene, either from another wild-type (wt) AAV sequence or an unrelated protein. In some embodiments, a chimeric capsid is produced using a naturally existing serotype as a template. In some embodiments, a chimeric capsid is produced using a serotype that is mutated relative to a wild type as a template. In some embodiments, a chimeric capsid can comprise at least one capsid polypeptide from an AAV serotype comprising AAV1, AAV2, AAV3, AAV4, AAV5, AAV6, AAV7, AAV8, AAV9, AAV10, AAV 11, or AAV12. In some embodiments, a viral vector provided herein comprises a polypeptide comprising a VP1 from an AAV serotype comprising AAV1, AAV2, AAV3, AAV4, AAV5, AAV6, AAV7, AAV8, AAV9, AAV10, AAV 11, or AAV12. In other embodiments, a viral vector provided herein comprises a polypeptide comprising a VP2 from an AAV comprising AAV1, AAV2, AAV3, AAV4, AAV5, AAV6, AAV7, AAV8, AAV9, AAV10, AAV 11, or AAV12. In some embodiments, a viral vector provided herein comprises a polypeptide comprising a VP3 from an AAV serotype comprising AAV1, AAV2, AAV3, AAV4, AAV5, AAV6, AAV7, AAV8, AAV9, AAV10, AAV11, or AAV12.

In some embodiments, the AAV particle described herein targets a cell. In some embodiments, the AAV particle is capable of transducing a particular cell type. In some embodiments, the cell is a blood cell. The blood cell can be a leukocyte. The leukocyte can be a T cell, or a particular type of T cell. According, in some embodiments, the AAV particle is capable of transducing a naïve T cell. In some embodiments, the AAV particle is capable of transducing a cytotoxic T cell. In some embodiments, the AAV particle is capable of transducing a helper T cell. Details of selecting an AAV vector based on the target cell are well known in the art and provided in, for example, Viney et al., (2021), J. Virol., 95(7):e02023-20, Mietzsch et al., (2021), J Virol. 95(19):e0077321 and Benskey et al., (2019), Methods Mol Biol., 1937:3-26, each of which is incorporated by reference in their entireties.

### Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 Aug; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### Effector Proteins

Provided herein are vectors encoding an effector protein or methods that use an effector protein. In some embodiments, an effector protein provided herein interacts with a guide nucleic acid to form a complex. In some embodiments, an interaction between the complex and a target nucleic acid comprises one or more of: recognition of a protospacer adjacent motif (PAM) sequence within the target nucleic acid by the effector protein, hybridization of the guide nucleic acid to the target nucleic acid, modification of the target nucleic acid by the effector protein, or combinations thereof. In some embodiments, recognition of a PAM sequence within a target nucleic acid may direct the modification activity of an effector protein. In some embodiments, recognition of a PAM sequence adjacent to a target nucleic acid may direct the modification activity of an effector protein.

Modification activity of an effector protein or an engineered protein described herein may be cleavage activity, binding activity, insertion activity, substitution activity, and the like. Modification activity of an effector protein may result in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, an ability of an effector protein to edit a target nucleic acid may depend upon the effector protein being complexed with a guide nucleic acid, the guide nucleic acid being hybridized to a target sequence of the target nucleic acid, the distance between the target sequence and a PAM sequence, or combinations thereof. A target nucleic acid comprises a target strand and a non-target strand. Accordingly, in some embodiments, the effector protein may edit a target strand and/or a non-target strand of a target nucleic acid.

The modification of the target nucleic acid generated by an effector protein may, as a non-limiting example, result in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization). Accordingly, in some embodiments, provided herein are methods of editing a target nucleic acid using an effector protein of the present disclosure, or compositions or systems thereof. Also provided herein are methods of modulating expression of a target nucleic acid using an effector protein of the present disclosure, or compositions or systems thereof. Further provided herein are methods of modulating the activity of a translation product of a target nucleic acid using an effector protein of the present disclosure, or compositions or systems thereof.

In some embodiments, the complex interacts with a target nucleic acid In some embodiments, the vectors comprise viral vectors or nonviral vectors. Accordingly, provided herein are viral vectors encoding an effector protein or methods that use an effector protein. In general, the effector protein is a Cas effector protein. The effector proteins can be small, which are beneficial for nucleic acid editing. The small nature of these effector proteins allow for them to be more easily packaged and delivered with higher efficiency in the context of genome editing.

In some embodiments, the length of the effector protein is at least about 300, at least about 350, at least about 400, at least about 450 linked amino acids. In some embodiments, the length of the effector protein is at least 400 linked amino acid residues. In some embodiments, the length of the effector protein is less than less than about 400, less than about 450, less than about 500, less than about 550, less than about 600 linked amino acid residues.

In some embodiments, the length of the effector protein is about 300 to about 600 linked amino acid residues. In some embodiments, the length of the effector protein is about 400 to about 600 linked amino acid residues. In some embodiments, the length of the effector protein is about 450 to about 550 linked amino acids. In some embodiments, the length of the effector protein is about 420 to about 480 linked amino acids. In some embodiments, the length of the effector protein is about 400 to about 420, about 420 to about 440, about 440 to about 460, about 460 to about 480, about 480 to about 500, about 500 to about 520, about 520 to about 540, about 540 to about 560, about 560 to about 580, about 580 to about 600 linked amino acids.

In some embodiments, the effector protein is a Type V Cas protein. In some embodiments, the effector protein is a Type VI Cas protein. In general, a Type V Cas effector protein comprises a RuvC domain but lacks an HNH domain. In some embodiments, the RuvC domain of the Type V Cas effector protein comprises three RuvC subdomains. In some embodiments, the three RuvC subdomains are located within the C-terminal half of the Type V Cas effector protein. In some embodiments, none of the RuvC subdomains are located at the N terminus of the protein. In some embodiments, the RuvC subdomains are contiguous. In some embodiments, there are zero to about 50 amino acids between the first and second RuvC subdomains. In some embodiments, there are zero to about 50 amino acids between the second and third RuvC subdomains.

In some embodiments, the effector proteins comprise a RuvC domain (e.g., a partial RuvC domain). In some embodiments, the RuvC domain can be defined by a single, contiguous sequence, or a set of partial RuvC domains that are not contiguous with respect to the primary amino acid sequence of the protein. An effector protein of the present disclosure can include multiple partial RuvC domains, which can combine to generate a RuvC domain with substrate binding or catalytic activity. For example, an effector protein can include three partial RuvC domains (RuvC-I, RuvC-II, and RuvC-III, also referred to herein as subdomains) that are not contiguous with respect to the primary amino acid sequence of the effector protein, but form a RuvC domain once the protein is produced and folds. In some embodiments, effector proteins comprise a recognition domain with a binding affinity for a guide nucleic acid or for a guide nucleic acid-target nucleic acid heteroduplex. In some embodiments, the effector protein does not comprise a zinc finger domain. In some embodiments, the effector protein does not comprise an HNH domain.

In some embodiments, the effector protein is a Cas14 effector protein. In some embodiments, the effector protein is a Cas12 effector protein. In some embodiments, the effector protein is a CasΦ effector protein described herein. In some embodiments, the effector protein is a CasM effector described herein. In some embodiments, the Cas12 effector is a Cas12a, Cas12b, Cas12c, Cas12d, a Cas12e or a Cas12j effector. In some embodiments, the effector protein is a Cas 13 effector. In some embodiments, the Cas13 effector is a Cas13a, a Cas13b, a Cas 13c or a Cas 13d effector.

Provided herein, in some embodiments, are viral vectors that comprise a nucleotide sequence encoding an effector protein. Also provided herein, in some embodiments, are methods that use an effector protein. TABLE 1 provides illustrative amino acid sequences of effector proteins for the viral vectors and methods described herein. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences recited in TABLE 1. In some embodiments, an effector protein provided herein comprises an amino acid sequence that is at least 65% identical to any one of the sequences as set forth in TABLE 1. In some embodiments, an effector protein provided herein comprises an amino acid sequence that is at least 70% identical to any one of the sequences as set forth in TABLE 1. In some embodiments, an effector protein provided herein comprises an amino acid sequence that is at least 75% identical to any one of the sequences as set forth in TABLE 1. In some embodiments, an effector protein provided herein comprises an amino acid sequence that is at least 80% identical to any one of the sequences as set forth in TABLE 1. In some embodiments, an effector protein provided herein comprises an amino acid sequence that is at least 85% identical to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% identical to any one of the sequences as set forth in TABLE 1. In some embodiments, an effector protein provided herein comprises an amino acid sequence that is at least 97% identical to any one of the sequences as set forth in TABLE 1. In some embodiments, an effector protein provided herein comprises an amino acid sequence that is at least 98% identical to any one of the sequences as set forth in TABLE 1. In some embodiments, an effector protein provided herein comprises an amino acid sequence that is at least 99% identical to any one of the sequences as set forth in TABLE 1. In some embodiments, an effector protein provided herein comprises an amino acid sequence that is identical to any one of the sequences as set forth in TABLE 1.

In some embodiments, compositions, systems and methods described herein comprise an effector protein, or a nucleic acid encoding the effector protein, wherein the amino acid sequence of the effector protein comprises at least about 200 contiguous amino acids or more of any one of the sequences recited in TABLE 1. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, at least about 620 contiguous amino acids, at least about 640 contiguous amino acids, at least about 660 contiguous amino acids, at least about 680 contiguous amino acids, at least about 700 contiguous amino acids, or more of any one of the sequences of TABLE 1.

In some embodiments, compositions, systems and methods described herein comprise an effector protein or a nucleic acid encoding the effector protein, wherein the effector protein comprises a portion of any one of the sequences recited in TABLE 1. In some embodiments, the effector protein comprises a portion of any one of the sequences recited in TABLE 1, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of any one of the sequences recited in TABLE 1. In some embodiments, the effector protein comprises a portion of any one of the sequences recited in TABLE 1, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of any one of the sequences recited in TABLE 1.

In some embodiments, the effector protein comprises an amino acid sequence is at least 65%, at least 70%, at least 75%, at least 80%, at least 85%, at least 90%, at least 95%, at least 97%, or at least 98%, at least 99%, or 100% identical to any one of SEQ ID NOs: 1-203, 2435, 2592, 2599 and 2601. In some embodiments, the effector protein comprises an amino acid sequence that has at least about 85% sequence identity to a sequence selected from the group consisting of SEQ ID NOs: 1-45, 2435, 2599 and 2601. In some embodiments, the effector protein comprises an amino acid sequence that has at least about 90% sequence identity to a sequence selected from the group consisting of SEQ ID NOs: 1-45, 2435, 2599 and 2601. In some embodiments, the effector protein comprises an amino acid sequence that has at least about 95% sequence identity to a sequence selected from the group consisting of SEQ ID NOs: 1-45, 2435, 2599 and 2601. In some embodiments, the effector protein comprises an amino acid sequence that has at least about 98% sequence identity to a sequence selected from the group consisting of SEQ ID NOs: 1-45, 2435, 2599 and 2601. In some embodiments, the effector protein comprises an amino acid sequence that has at least about 99% sequence identity to a sequence selected from the group consisting of SEQ ID NOs: 1-45, 2435, 2599 and 2601. In some embodiments, the effector protein comprises an amino acid sequence that is identical to a sequence selected from the group consisting of SEQ ID NOs: 1-45, 2435, 2599 and 2601. In some embodiments, the effector protein comprises an amino acid sequence that has at least about 85% sequence identity to a sequence selected from the group consisting of SEQ ID NOs: 46-94 and 2592. In some embodiments, the effector protein comprises an amino acid sequence that has at least about 90% sequence identity to a sequence selected from the group consisting of SEQ ID NOs: 46-94 and 2592. In some embodiments, the effector protein comprises an amino acid sequence that has at least about 95% sequence identity to a sequence selected from the group consisting of SEQ ID NOs: 46-94 and 2592. In some embodiments, the effector protein comprises an amino acid sequence that has at least about 98% sequence identity to a sequence selected from the group consisting of SEQ ID NOs: 46-94 and 2592. In some embodiments, the effector protein comprises an amino acid sequence that has at least about 99% sequence identity to a sequence selected from the group consisting of SEQ ID NOs: 46-94 and 2592. In some embodiments, the effector protein comprises an amino acid sequence that is identical to a sequence selected from the group consisting of SEQ ID NOs: 46-94 and 2592. In some embodiments, the effector protein comprises an amino acid sequence that has at least about 85%, at least about 90%, at least about 95%, at least about 98%, at least about 99% sequence identity, or is identical, to a sequence selected from the group consisting of SEQ ID NOs: 95-203.

In some embodiments, compositions, systems, and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the sequences as set forth in TABLE 1. In some embodiments, an effector protein provided herein comprises an amino acid sequence that is at least 80% similar to any one of the sequences as set forth in TABLE 1. In some embodiments, an effector protein provided herein comprises an amino acid sequence that is at least 85% similar to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% similar to any one of the sequences as set forth in TABLE 1. In some embodiments, an effector protein provided herein comprises an amino acid sequence that is at least 97% similar to any one of the sequences as set forth in TABLE 1. In some embodiments, an effector protein provided herein comprises an amino acid sequence that is at least 98% identical to any one of the sequences as set forth in TABLE 1. In some embodiments, an effector protein provided herein comprises an amino acid sequence that is at least 99% similar to any one of the sequences as set forth in TABLE 1. In some embodiments, an effector protein provided herein comprises an amino acid sequence that is 100% similar to any one of the sequences as set forth in TABLE 1.

In some embodiments, when describing a certain percent (%) similarity in the context of an amino acid sequence, reference may be made to a value that is calculated by dividing a similarity score by the length of the alignment. In some embodiments, the similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, in some embodiments, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, in some embodiments, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. In some embodiments, the % similarity can also be calculated using commercially available programs, such as the Geneious Prime software given the parameters matrix =BLOSUM62 and threshold ≥1.

In some embodiments, compositions, systems, and methods described herein comprise an effector protein, or a nucleic acid encoding the effector protein, wherein the effector protein comprises one or more amino acid alterations relative to any one of the sequences recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, conservative deletions, non-conservative deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences recited in TABLE 1.

Effector proteins disclosed herein can function as an endonuclease that catalyzes cleavage at a specific position (e.g., at a specific nucleotide within a target sequence) in a target nucleic acid. The target nucleic acid can be single stranded RNA (ssRNA), double stranded DNA (dsDNA) or single-stranded DNA (ssDNA). In some embodiments, the target nucleic acid is single-stranded DNA. In some embodiments, the target nucleic acid is single-stranded RNA. The effector proteins can provide cis cleavage activity, trans cleavage activity, nickase activity, or a combination thereof. Cis cleavage activity is cleavage of a target nucleic acid that is hybridized to a guide nucleic acid (e.g., a dual gRNA or a sgRNA), wherein cleavage occurs within or directly adjacent to the region of the target nucleic acid that is hybridized to guide nucleic acid. Trans cleavage activity is cleavage of ssDNA or ssRNA that is near, but not hybridized to the guide nucleic acid. Trans cleavage activity is triggered by the hybridization of the guide nucleic acid to the target nucleic acid. Nickase activity is a selective cleavage of one strand of a dsDNA.

### Engineered Proteins

In some embodiments, effector proteins disclosed herein are engineered proteins. Engineered proteins are not identical to a naturally-occurring protein. Such an engineered protein can include one or more mutations, including an insertion, deletion or substitution (e.g., conservative or non-conservative substitution). An engineered protein, in some embodiments, includes at least one mutation relative to a reference protein (e.g., a naturally-occurring protein). In some embodiments, an engineered protein includes at least 1, at least 2, at least 3, at least 4, at least 5, at least 6, at least 7, at least 8, at least 9, at least 10, at least 15, at least 20, at least 25 or at least 30 mutations relative to a reference protein (e.g., a naturally-occurring protein). In some embodiments, an engineered protein includes no more than 10, 20, 30, 40, or 50 mutations relative to a reference protein (e.g., a naturally-occurring protein). Engineered proteins may not comprise an amino acid sequence that is identical to that of a naturally-occurring protein. In some embodiments, the amino acid sequence of an engineered protein is not identical to that of a naturally occurring protein. Engineered proteins may provide an increased activity relative to a naturally occurring protein. Engineered proteins may provide a reduced activity relative to a naturally occurring protein. The activity may be nuclease activity. The activity may be nickase activity. The activity may be nucleic acid binding activity. Accordingly, in some embodiments, engineered proteins may provide enhanced activity (e.g., enhanced binding of a guide nucleic acid, and/or target nucleic acid, enhanced nuclease activity, enhanced nickase activity, etc.) as compared to a naturally-occurring counterpart. In such embodiments, the effector protein may have a 1%, 5%, 10%, 20%, 30%, 40%, 50%, 60%, 70%, 80%, 90%, 100%, 120%, 140%, 160%, 180%, 200%, or more, increased activity relative to a naturally-occurring counterpart. Alternatively, in some embodiments, engineered proteins may provide reduced activity (e.g., reduced binding of a guide nucleic acid, and/or target nucleic acid, reduced nuclease activity, reduced nickase activity, etc.) relative to a naturally occurring effector protein. In such embodiments, the engineered proteins may have a 100%, 90%, 80%, 70%, 60%, 50%, 40%, 30%, 20%, 10%, 5%, 1%, or less, decreased activity relative to a naturally occurring counterpart.

In some embodiments, effector proteins disclosed herein are engineered proteins. Engineered proteins are not identical to a naturally occurring protein. Engineered proteins can provide enhanced nuclease or nickase activity as compared to a naturally occurring nuclease or nickase. Effector proteins may provide enhanced nucleic acid binding activity (e.g., enhanced binding of a guide nucleic acid, and/or target nucleic acid) as compared to a naturally-occurring counterpart. An effector protein may have a 1%, 5%, 10%, 20%, 30%, 40%, 50%, 60%, 70%, 80%, 90%, 100%, 120%, 140%, 160%, 180%, 200%, or more, increase of the activity (e.g., nuclease activity, nickase activity, binding activity) of a naturally-occurring counterpart. An engineered protein can comprise a modified form of a wildtype counterpart protein.

In some embodiments, effector proteins comprise at least one amino acid change (e.g., deletion, insertion, or substitution) that reduces the nucleic acid-cleaving activity of the effector protein relative to the wildtype counterpart. For example, a nuclease domain (e.g., RuvC domain) of an effector protein can be deleted or mutated relative to a wildtype counterpart effector protein so that it is no longer functional or comprises reduced nuclease activity. The effector protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered proteins can have no substantial nucleic acid-cleaving activity. Engineered proteins can be enzymatically inactive or “dead,” that is it can bind to a nucleic acid but not cleave it. An enzymatically inactive protein can comprise an enzymatically inactive domain (e.g. inactive nuclease domain). Enzymatically inactive can refer to an activity less than 1%, less than 2%, less than 3%, less than 4%, less than 5%, less than 6%, less than 7%, less than 8%, less than 9%, or less than 10% activity compared to the wild-type counterpart. A dead protein can associate with a guide nucleic acid to activate or repress transcription of a target nucleic acid sequence. In some embodiments, the enzymatically inactive protein is fused with a protein comprising recombinase activity.

In some embodiments, effector proteins comprise at least one amino acid change (e.g., deletion, insertion, or substitution) that increases the nucleic acid-cleaving activity of the effector protein relative to the wildtype counterpart. The effector protein can provide at least about 20%, at least about 30%, at least about 40%, at least about 50% at least about 60%, at least about 70%, at least about 80%, at least about 90%, or about 100% more nucleic acid-cleaving activity relative to that of the wild-type counterpart. The effector protein can provide at least about 2 fold, at least about 3 fold, at least about 4 fold, at least about 5 fold, at least about 6 fold, at least about 7 fold, at least about 8 fold, at least about 9 fold or at least about 10 fold more nucleic acid-cleaving activity relative to that of the wild-type counterpart.

In some embodiments, the effector protein or corresponding mRNA comprises an NLS and/or a polyA tail, respectively. An NLS is a sequence that tags a protein for import into the cell nucleus. There are many NLS described in the art. The length of the NLS can be about 5 to about 100 amino acids. The length of the NLS can be about 10 amino acids to about 20, about 30, about 40, about 50, or about 60 amino acids. The NLS can be located at the 5′ end of the effector protein. The NLS can be located at the 3′ end of the effector protein. The NLS can be located at an internal site of the effector protein (e.g., between the 5′ and 3′ end of the effector protein, but not at the 5′ or 3′ end of the effector protein). In general, the viral vector encodes an mRNA that is translated into the effector protein. In some embodiments, the mRNA comprises a polyA tail. This can increase the stability of the effector protein mRNA, thereby increasing production of Cas effector protein.

### Fusion Proteins

In some embodiments, a viral vector described herein comprises a nucleotide sequence that encodes an effector protein or a method described herein uses an effector protein, wherein the effector protein is a fusion protein. Such an effector protein can comprise a Cas effector protein and a fusion partner protein. A fusion partner protein is also simply referred to herein as a fusion partner. The fusion partner can comprise a protein or a functional domain thereof. Non-limiting examples of fusion partners include a protein having enzymatic activity that modifies a target nucleic acid and a signaling peptide, e.g., a nuclear localization signal (NLS). Accordingly, in some embodiments, fusion partners provide enzymatic activity that modifies a target nucleic acid. Such enzymatic activities include, but are not limited to, nuclease activity, DNA repair activity, DNA damage activity, integrase activity, transposase activity, recombinase activity, polymerase activity, ligase activity, and helicase activity. In some embodiments, the fusion partner comprises an RNA splicing factor. In some embodiments, any of the effector protein of the present disclosure (e.g., any of the effector proteins of TABLE 1 or fragments or variants thereof) can include a nuclear localization signal (NLS). In some cases, one or more NLS are fused or linked to the N-terminus of the effector protein. In some embodiments, one or more NLS are fused or linked to the C-terminus of the effector protein. In some embodiments, one or more NLS are fused or linked to the N-terminus and the C-terminus of the effector protein.

In some embodiments, an effector protein described herein comprises 1, 2, 3, 4, 5, 6, 7, 8, 9, 10, or more NLS at or near the N-terminus, 1, 2, 3, 4, 5, 6, 7, 8, 9, 10, or more NLS at or near the C-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLS present in one or more copies. In some embodiments, a NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In some embodiments, a NLS described herein comprises a heterologous polypeptide sequence recited in TABLE 1.1. In some embodiments, effector proteins described herein comprise an amino acid sequence that is at least about 65%, at least about 70%, at least about 75%, at least about 80%, at least about 85%, at least about 90%, at least about 95%, at least about 97%, at least about 98%, at least about 99%, or about 100% identical to any one of the sequences recited in TABLE 1 and further comprises one or more of the sequences set forth in TABLE 1.1. In some embodiments, a heterologous peptide described herein may be a fusion partner as described en supra.

In some embodiments, the link between the NLS and the effector protein comprises a tag. In some cases, said NLS can have a sequence of KRPAATKKAGQAKKKKEF (SEQ ID NO: 1584). The NLS can be selected to match the cell type of interest, for example several NLSs are known to be functional in different types of eukaryotic cell e.g. in mammalian cells. Suitable NLSs include the SV40 large T antigen NLS (PKKKRKV, SEQ ID NO: 1585) and the c-Myc NLS (PAAKRVKLD, SEQ ID NO: 1586). In some embodiments, an NLS can be the SV40 large T antigen NLS or the c-Myc NLS. NLSs that are functional in plant cells are described in Chang et al., (Plant Signal Behav. 2013 October; 8(10):e25976). In some embodiments, the nucleoplasmin NLS (KRPAATKKAGQAKKKKEF (SEQ ID NO: 1584)) is linked or fused to the C-terminus of the effector protein. In some embodiments, the SV40 NLS (PKKKRKVGIHGVPAA) (SEQ ID NO: 1587) is linked or fused to the N-terminus of the effector protein. In some embodiments, the nucleoplasmin NLS (SEQ ID NO: 1584) is linked or fused to the C-terminus of the programmable CasΦ nuclease and the SV40 NLS (SEQ ID NO: 1587) is linked or fused to the N-terminus of the effector protein.

### Multimeric Complexes

In some embodiments, viral vectors described herein comprise a nucleotide sequence that encodes an effector protein or methods described herein use an effector protein, wherein the effector protein forms a multimeric complex with another protein. In general, a multimeric complex comprises multiple proteins that non-covalently interact with one another. In some embodiments, the multimeric complex comprises a first effector protein and a second effector protein, wherein the first effector protein and the second effector protein are the same. In some embodiments, the multimeric complex comprises a first effector protein and a second effector protein, wherein the first effector protein and the second effector protein are different. A multimeric complex can comprise enhanced activity relative to the activity of any one of its effector proteins alone. For example, a multimeric complex comprising two effector proteins can comprise greater nucleic acid binding affinity, cis cleavage activity, and/or trans cleavage activity, than that of either of the effector proteins provided in monomeric form. A multimeric complex can have an affinity for a target region of a target nucleic acid and is capable of catalytic activity (e.g., cleaving, nicking or modifying the nucleic acid) at or near the target region. Multimeric complexes can be activated when complexed with a guide nucleic acid. Multimeric complexes can be activated when complexed with a guide nucleic acid and a target nucleic acid. In some embodiments, the multimeric complex cleaves the target nucleic acid. In some embodiments, the multimeric complex nicks the target nucleic acid.

In some embodiments, multimeric complexes comprise at least one effector protein comprising an amino acid sequence with at least 65%, at least 70%, at least 75%, at least 80%, at least 85%, at least 90%, at least 95% or 100% identity to any one of the sequences of TABLE 1. In some embodiments, the multimeric complex is a dimer comprising two effector proteins of identical amino acid sequences. In some embodiments, the multimeric complex comprises a first effector protein and a second effector protein, wherein the amino acid sequence of the first effector protein is at least 90%, at least 92%, at least 94%, at least 96%, at least 98% identical, or at least 99% identical to the amino acid sequence of the second effector protein.

In some embodiments, the multimeric complex is a heterodimeric complex comprising at least two effector proteins of different amino acid sequences. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein.

In some embodiments, a multimeric complex comprises at least two effector proteins. In some embodiments, a multimeric complex comprises more than two effector proteins. In some embodiments, a multimeric complex comprises two, three or four effector proteins. In some embodiments, at least one effector protein of the multimeric complex comprises an amino acid sequence with at least 65%, at least 70%, at least 75%, at least 80%, at least 85%, at least 90%, at least 95% or 100% identity to any one of the sequences of TABLE 1. In some embodiments, each effector protein of the multimeric complex independently comprises an amino acid sequence with at least 65%, at least 70%, at least 75%, at least 80%, at least 85%, at least 90%, at least 95% or 100% identity to any one of the sequences of TABLE 1.

Effector proteins disclosed herein can also function as an endonuclease for the production of a guide nucleic acid. Accordingly, in some embodiments, an effector protein or a multimeric complex thereof cleaves a precursor crRNA (“pre-crRNA”) to produce a guide RNA, also referred to as a “mature guide RNA.” For example, when a vector (e.g., viral vector or non-viral vector) described herein includes a promoter that produces the guide nucleic acid for targeting the effector protein to the TRAC gene, the B2M gene and the CIITA gene in the same RNA transcript, the effector protein can process the RNA transcript to generate the individual guide nucleic acids for targeting the effector protein to the TRAC gene, the B2M gene and the CIITA gene. Alternatively, if the vector (e.g., viral vector or non-viral vector) is RNA, the nucleotide sequences for producing the guide nucleic acids can be considered a pre-crRNA, which can result in a guide nucleic acid when cleaved by an effector protein. An effector protein that cleaves pre-crRNA to produce a mature guide RNA is said to have pre-crRNA processing activity. In some embodiments, a repeat region of a guide RNA comprises mutations or truncations relative to respective regions in a corresponding pre-crRNA.

### Protospacer Adjacent Motif (Pam) Sequences

Effector proteins of the present disclosure may cleave or nick a target nucleic acid within or near a protospacer adjacent motif (PAM) sequence of the target nucleic acid. In some embodiments, the target nucleic acid is a double stranded nucleic acid comprising a target strand and a non-target strand. In some embodiments, cleavage occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, effector proteins described herein recognize a PAM sequence. In some embodiments, recognizing a PAM sequence comprises interacting with a sequence adjacent to the PAM. In some embodiments, a target nucleic acid comprises a target sequence that is adjacent to a PAM sequence. In some embodiments, the effector protein does not require a PAM to bind and/or cleave a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP cleaves the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, such a PAM described herein is directly adjacent to the target sequence on the target strand or the non-target strand. In some embodiments, an RNP cleaves the target strand or the non-target strand. In some embodiments, the RNP cleaves both, the target strand and the non-target strand. In some embodiments, an RNP recognizes the PAM sequence, and hybridizes to a target sequence of the target nucleic acid. In some embodiments, the RNP cleaves the target nucleic acid, wherein the RNP has recognized the PAM sequence and is hybridized to the target sequence.

An effector protein of the present disclosure, or a multimeric complex thereof, may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence.

In some embodiments, an effector protein or a multimeric complex thereof recognizes a PAM on a target nucleic acid. In some cases, multiple effector proteins of the multimeric complex recognize a PAM on a target nucleic acid. In some cases, only one effector protein of the multimeric complex recognizes a PAM on a target nucleic acid. In some embodiments, at least two of the multiple effector proteins recognize the same PAM sequence. In some embodiments, at least two of the multiple effector proteins recognize different PAM sequences. In some embodiments, only one effector protein of the multimeric complex recognizes a PAM on a target nucleic acid. In some cases, the PAM is 3′ to the spacer region of the guide nucleic acid. In some cases, the PAM is directly 3′ to the spacer region of the guide nucleic acid. In some cases, the PAM sequence comprises a sequence described herein.

Effector proteins of the present disclosure can recognize a wild type PAM or a mutant PAM in a target DNA. In some embodiments, the effector protein is a CasΦ effector protein of the present disclosure that recognizes a PAM of 5′-TBN-3′, where B is one or more of C, G, or, T. For example, CasΦ effector protein of the present disclosure can recognize a PAM of 5′-TTTN-3′, wherein N is any nucleotide. As another example, CasΦ effector protein of the present disclosure can recognize a PAM of 5′-TTN-3′, wherein N is any nucleotide. In some embodiments, the PAM is 5′-TTTA-3′, 5′-GTTK-3′, 5′-VTTK-3′, 5′-VTTS-3′, 5′-TTTS-3′ or 5′-VTTN-3′, wherein K is G or T, V is A, C or G, S is C or G and N is any nucleotide. In some embodiments, the PAM is 5′-GTTB-3′, wherein B is C, G, or, T. In some embodiments of the present disclosure, the CasΦ effector protein can recognize a PAM of 5′-NTTN-3′, wherein N is any nucleotide. Other effector proteins disclosed herein (e.g., effector proteins of SEQ ID NO: 95-203), or a multimeric complex thereof, can recognize a different PAM sequence in the target nucleic acid. In some cases, the PAM sequence is 5′-CTT-3′. In some cases, the PAM sequence is 5′-CC-3′. In some cases, the PAM sequence is 5′-TCG-3′. In some cases, the PAM sequence is 5′-GCG-3′. In some cases, the PAM sequence is 5′-TTG-3′. In some cases, the PAM sequence is 5′-GTG-3′. In some cases, the PAM sequence is 5′-ATTA-3′. In some cases, the PAM sequence is 5′-ATTG-3′. In some cases, the PAM sequence is 5′-GTTA-3′. In some cases, the PAM sequence is 5′-GTTG-3′. In some cases, the PAM sequence is 5′-TC-3′. In some cases, the PAM sequence is 5′-ACTG-3′. In some cases, the PAM sequence is 5′-GCTG-3′. In some cases, the PAM sequence is 5′-TTC-3′. In some cases, the PAM sequence is 5′-TTT-3′.

Effector proteins of the present disclosure, dimers thereof, and multimeric complexes thereof can cleave or nick a target nucleic acid within or near a protospacer adjacent motif (PAM) sequence of the target nucleic acid. In some embodiments, cleavage occurs within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 nucleotides of a 5′ or 3′ terminus of a PAM sequence. As a result of this cleavage, in some embodiments, an indel occurs within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 nucleotides of the PAM sequence. A target nucleic acid can comprise a PAM sequence adjacent to a sequence that is complementary to a guide nucleic acid spacer region.

### Guide Nucleic Acids

Provided herein are vectors that include nucleotide sequences that, when transcribed and/or cleaved by the effector protein, produces one or more engineered guide nucleic acids. In some embodiments, the vectors comprise viral vectors or nonviral vectors. Accordingly, provided herein are viral vectors that include nucleotide sequences that, when transcribed and/or cleaved by the effector protein, produces one or more engineered guide nucleic acids. Guide nucleic acids, when composed of RNA, are often referred to as a “guide RNAs.” However, a guide nucleic acid can comprise deoxyribonucleotides. Accordingly, in some embodiments, guide nucleic acids can comprise DNA, RNA, or a combination thereof (e.g., RNA with a thymine base). The term “guide RNA,” as well as crRNA and tracrRNA sequence, include guide nucleic acids comprising DNA bases, RNA bases and modified nucleobases.

A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). A guide nucleic acid may be chemically synthesized or recombinantly produced by any suitable methods. Guide nucleic acids can include a chemically modified nucleobase or phosphate backbone. In some embodiments, guide nucleic acids described herein comprises one or more 2′O-methyl modified nucleotides. In some embodiments, guide nucleic acids described herein comprises at least one 2′O-methyl modified nucleotides. In some embodiments, guide nucleic acids described herein comprises one, two, three, four or five 2′O-methyl modified nucleotides. In some embodiments, guide nucleic acids described herein comprises one, two, three, four or five contiguous 2′O-methyl modified nucleotides. In some embodiments, 3′ end of any one of the guide nucleic acids described herein comprises one, two, three, four or five contiguous 2′O-methyl modified nucleotides. In some embodiments, 5′ end of any one of the guide nucleic acids described herein comprises one, two, three, four or five contiguous 2′O-methyl modified nucleotides.

In general, the guide nucleic acid comprises a nucleotide sequence that is at least 80%, at least 85%, at least 90%, at least 95%, at least 98%, at least 99%, or 100% complementary to the target sequence. In some embodiments, the guide nucleic acid comprises at least 10 contiguous nucleotides that are complementary to the target sequence in the target nucleic acid. In some embodiments, guide nucleic acid comprises a spacer sequence that is at least 80%, at least 85%, at least 90%, at least 95%, at least 98%, at least 99%, or 100% complementary to the target sequence.

In some embodiments, the guide nucleic acid can comprise a first region complementary to a target sequence (FR1) and a second region that is not complementary to the target sequence (FR2). In some embodiments, FR1 is located 5′ to FR2 (FR1-FR2). In some embodiments, FR2 is located 5′ to FR1 (FR2-FR1).

In some embodiments, the FR1 comprises one or more repeat sequences, handle sequence, or intermediary sequence. In some embodiments, an effector protein binds to at least a portion of the FR1. In some embodiments, the FR2 comprises a spacer sequence, wherein the spacer sequence can interact in a sequence-specific manner with (e.g., has complementarity with, or can hybridize to a target sequence in) a target nucleic acid.

In some embodiments, the first region, the second region, or both may be about 8 nucleic acids, about 10 nucleic acids, about 12 nucleic acids, about 14 nucleic acids, about 16 nucleic acids, about 18 nucleic acids, about 20 nucleic acids, about 22 nucleic acids, about 24 nucleic acids, about 26 nucleic acids, about 28 nucleic acids, about 30 nucleic acids, about 32 nucleic acids, about 34 nucleic acids, about 36 nucleic acids, about 38 nucleic acids, about 40 nucleic acids, about 42 nucleic acids, about 44 nucleic acids, about 46 nucleic acids, about 48 nucleic acids, or about 50 nucleic acids long.

In some embodiments, the first region, the second region, or both may be from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, or from about 8 to about 50 nucleic acids long.

In some embodiments, the first region, the second region, or both may comprise a GC content of about 1%, about 5%, about 10%, about 15%, about 20%, about 25%, about 30%, about 40%, about 50%, about 60%, about 70%, about 80%, about 90%, about 95%, or about 99%. In some embodiments, the first region, the second region, or both may comprise a GC content of from about 1% to about 95%, from about 5% to about 90%, from about 10% to about 80%, from about 15% to about 70%, from about 20% to about 60%, from about 25% to about 50%, or from about 30% to about 40%.

In some embodiments, the first region, the second region, or both may have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region, the second region, or both may have a melting temperature of from about 35° C. to about 40° C., from about 35° C. to about 45° C., from about 35° C. to about 50° C., from about 35° C. to about 55° C., from about 35° C. to about 60° C., from about 35° C. to about 65° C., from about 35° C. to about 70° C., from about 35° C. to about 75° C., from about 35° C. to about 80° C., or from about 35° C. to about 85° C.

In some embodiments, the compositions, systems, devices, kits, and methods of the present disclosure further comprise an additional nucleic acid, wherein a portion of the additional nucleic acid at least partially hybridizes to the first region of the guide nucleic acid. In some embodiments, the additional nucleic acid is at least partially hybridized to the 5′ end of the second region of the guide nucleic acid. In some embodiments, an unhybridized portion of the additional nucleic acid, at least partially, interacts with an effector protein or polypeptide. In some embodiments, the compositions, systems, devices, kits, and methods of the present disclosure comprise a dual nucleic acid system comprising the guide nucleic acid and the additional nucleic acid as described herein.

In general, a guide nucleic acid is a nucleic acid molecule that binds to an effector protein (e.g., a Cas effector protein), thereby forming a RNP complex. In some embodiments, when in a complex, at least a portion of the complex may bind, recognize, and/or hybridize to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by hybridizing to different target sequences. In some embodiments, a first guide nucleic acid may hybridize within a location of the target nucleic acid that is different from where a second guide nucleic acid may hybridize the target nucleic acid. In some embodiments, the first loci and the second loci of the target nucleic acid may be located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart. In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, the engineered guide nucleic acid imparts activity or sequence selectivity to the effector protein. A guide nucleic acid can comprise a CRISPR RNA (crRNA), an associated tracrRNA sequence or a combination thereof. In general, the engineered guide nucleic acid comprises a crRNA that is at least partially complementary to a target nucleic acid. In some embodiments, the engineered guide nucleic acid comprises a tracrRNA sequence, at least a portion of which interacts with the effector protein. The tracrRNA can hybridize to a portion of the guide nucleic acid that does not hybridize to the target nucleic acid. In some embodiments, guide nucleic acids can be a guide RNA (gRNA). In some embodiments, the crRNA and tracrRNA sequence are provided as a single guide nucleic acid, also referred to as a single guide RNA (sgRNA). However, a guide RNA is not limited to ribonucleotides, but can comprise deoxyribonucleotides and other chemically modified nucleotides. The combination of a crRNA with a tracrRNA sequence can be referred to herein as a single guide RNA (sgRNA), wherein the crRNA and the tracrRNA sequence are covalently linked. In some embodiments, the crRNA and tracrRNA sequence are linked by a phosphodiester bond. In some embodiments, the crRNA and tracrRNA sequence are linked by one or more linked nucleotides. In some embodiments, a crRNA and tracrRNA function as two separate, unlinked molecules. A guide nucleic acid can comprise a naturally occurring guide nucleic acid. A guide nucleic acid can comprise a non-naturally occurring guide nucleic acid, including a guide nucleic acid that is designed to contain a chemical or biochemical modification.

In some embodiments, the length of the guide nucleic acid is not greater than about 40, about 45, about 50, about 55, about 60, about 65, about 70, about 75, about 80, about 85, about 90, about 95, about 100 linked nucleotides. In some embodiments, the length of the guide nucleic acid is about 30 to about 100 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides.

In some embodiments, the guide nucleic acid, in total (including any tracrRNA sequence), comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 linked nucleotides. In general, a guide nucleic acid comprises at least linked nucleotides. In some embodiments, a guide nucleic acid comprises at least 25 linked nucleotides in total. A guide nucleic acid can comprise 10 to 100 linked nucleotides in total. In some embodiments, the guide nucleic acid comprises or consists essentially of about 12 to about 80 linked nucleotides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleotides in total. In some embodiments, the guide nucleic acid has about 10 to about 60, about 20 to about 50, or about 30 to about 40 linked nucleotides in total.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, the viral vectors described herein and the non-viral vectors described herein include nucleotide sequences that produce guide nucleic acids that target the effector protein to different genes. In some embodiments, the methods described herein use guide nucleic acids that target the effector protein to different genes. Accordingly, in some embodiments, the nucleotide sequence that the effector protein binds is the same for the all of guide nucleic acids. Alternatively, in some embodiments, the nucleotide sequence that the effector protein binds is different for the guide nucleic acids. Thus, in some embodiments, the nucleotide sequence that the effector protein binds for the guide nucleic acids comprise at least about 90%, at least about 95%, at least about 98%, or at least 99% sequence identity to each other. Similarly, when the non-viral vector, the viral vectors or methods described herein produces or uses three or more guide nucleic acids, in some embodiments, two or more of the guide nucleic acids have the same nucleotide sequence that the effector protein binds, while one of the guide nucleic acids has a nucleotide sequence that the effector protein binds that is at least at least about 90%, at least about 95%, at least about 98%, or at least 99% sequence identity to the corresponding sequence in the other guide nucleic acids.

In some embodiments, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. In some cases, the segment of a guide nucleic acid that comprises a sequence that is reverse complementary to the target nucleic acid is 20 nucleotides in length. A guide nucleic acid can have at least 10, 11, 12, 13, 14, 15, 16, 17, 18, 19, 20, 21, 22, 23, 24, 25, 26, 27, 28, 29, or 30 nucleotides reverse complementary to a target nucleic acid. In some cases, the guide nucleic acid can be 10, 11, 12, 13, 14, 15, 16, 17, 18, 19, 20, 21, 22, 23, 24, 25, 26, 27, 28, 29, or 30 nucleotides in length. For example, a guide nucleic acid can have at least 10 nucleotides reverse complementary to a target nucleic acid. In some embodiments, a guide nucleic acid have from 10 to 50 nucleotides reverse complementary to a target nucleic acid. In some embodiments, a guide nucleic acid have at least 25 nucleotides reverse complementary to a target nucleic acid. In some cases, the guide nucleic acid has from exactly or about 12 nucleotides to about 80 nucleotides, from about 12 nucleotides to about 50 nucleotides, from about 12 nucleotides to about 45 nucleotides, from about 12 nucleotides to about 40 nucleotides, from about 12 nucleotides to about 35 nucleotides, from about 12 nucleotides to about 30 nucleotides, from about 12 nucleotides to about 25 nucleotides, from about 12 nucleotides to about 20 nucleotides, from about 12 nucleotides to about 19 nucleotides, from about 19 nucleotides to about 20 nucleotides, from about 19 nucleotides to about 25 nucleotides, from about 19 nucleotides to about 30 nucleotides, from about 19 nucleotides to about 35 nucleotides, from about 19 nucleotides to about 40 nucleotides, from about 19 nucleotides to about 45 nucleotides, from about 19 nucleotides to about 50 nucleotides, from about 19 nucleotides to about 60 nucleotides, from about 20 nucleotides to about 25 nucleotides, from about 20 nucleotides to about 30 nucleotides, from about 20 nucleotides to about 35 nucleotides, from about 20 nucleotides to about 40 nucleotides, from about 20 nucleotides to about 45 nucleotides, from about 20 nucleotides to about 50 nucleotides, or from about 20 nucleotides to about 60 nucleotides reverse complement to a target nucleic acid. In some cases, the guide nucleic acid has from about 10 nucleotides to about 60 nucleotides, from about 20 nucleotides to about 50 nucleotides, or from about 30 nucleotides to about 40 nucleotides reverse complementary to a target nucleic acid. It is understood that the sequence of a guide nucleic acid need not be 100% reverse complementary to that of its target nucleic acid to be specifically hybridizable, hybridizable, or bind specifically. For example, the guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid. The guide nucleic acid can hybridize with a target nucleic acid.

Guide nucleic acids, when complexed with an effector protein, can bring the effector protein into proximity of a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for effectuating the activity of a protein, polypeptide or peptide described herein, such as the nuclease activity of an effector protein.

The guide nucleic acid can hybridize to a target nucleic acid (e.g., a single strand of a target nucleic acid) or a portion thereof. The guide nucleic acid can hybridize to a target nucleic acid, such as a target sequence within the TRAC gene, B2M gene or the CIITA gene. Accordingly, in some embodiments, the guide nucleic acid guide nucleic acid comprises a sequence that is complementary to an equal length portion of a target sequence of the TRAC gene. In some embodiments, guide nucleic acid comprises a sequence that is complementary to an equal length portion of a target sequence of the B2M gene. In some embodiments, guide nucleic acid comprises a sequence that is complementary to an equal length portion of a target sequence of the CIITA gene. In some embodiments, guide nucleic acid comprises a sequence that is at least 90% identical to an equal length portion of a target sequence of the TRAC gene. In some embodiments, guide nucleic acid comprises a sequence that is at least 90% identical to an equal length portion of a target sequence of the B2M gene. In some embodiments, guide nucleic acid comprises a sequence that is at least 90% identical to an equal length portion of a target sequence of the CIITA gene.

In some embodiments, the guide nucleic acid comprises a nucleotide sequence described as described herein (e.g., TABLES 2—20, 23-26, 29-31, 36 and 38). Such nucleotide sequences described herein (e.g., TABLES 2—20, 23-26, 29-31, 36 and 38) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that produces a guide nucleic acid, such as a nucleotide sequence described herein for a viral vector. Similarly, disclosure of the nucleotide sequences described herein (e.g., TABLES 2—20, 23-26, 29-31, 36 and 38) also discloses the complementary nucleotide sequence, the reverse nucleotide sequence, and the reverse complement nucleotide sequence, any one of which can be a nucleotide sequence for use in a guide nucleic acid as described herein.

In some embodiments, the guide nucleic acid comprises a sequence that is at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56 or at least 57 contiguous nucleotides of a sequence described herein (e.g., TABLES 2—20, 23-26, 29-31, 36 and 38). In some embodiments, the guide nucleic acid comprises a sequence that is 30, 31, 32, 33, 34, 35, 36, 37, 38, 39, 40, 41, 42, 43, 44, 45, 46, 47, 48, 49, 50, 51, 52, 53, 54, 55, 56 or 57 contiguous nucleotides of a sequence described herein (e.g., TABLES 2—20, 23-26, 29-31, 36 and 38). In some embodiments, the guide nucleic acid comprises a sequence that is at least 30, at least 31, at least 32, at least 33, at least 34, at least 35, at least 36 or at least 37 contiguous nucleotides of a sequence described herein (e.g., TABLES 2—20, 23-26, 29-31, 36 and 38). In some embodiments, the guide nucleic acid comprises a sequence that is 30, 31, 32, 33, 34, 35, 36 or 37 contiguous nucleotides of a sequence described herein (e.g., TABLES 2—20, 23-26, 29-31, 36 and 38). In some embodiments, the guide nucleic acid comprises a repeat sequence described herein (e.g., TABLES 2-3) and/or a spacer sequence described herein (e.g., TABLES 5-16, 18-19, and 23).

In some embodiments, the effector protein disclosed herein is used in conjunction with a specific sequence (e.g., spacer or gRNA) for targeting an effector protein described herein to the TRAC gene, the B2M gene or the CIITA gene (e.g., TABLES 5-16, 19-20 or 29-31). In some embodiments, the guide nucleic acid comprises a nucleotide sequence that is at least 70%, at least 80%, at least 90%, at least 92%, at least 95%, at least 97%, at least 99%, or 100% identical to any one of sequences described herein (e.g., TABLES 5-20, 23-26, 29-31, 36 and 38) or a complement thereof.

In some embodiments, a guide nucleic acid comprises a nucleotide sequence for targeting the effector protein to the TRAC gene. In some embodiments, such a guide nucleic acid comprises a nucleotide sequence of any one of the sequences recited in TABLE 5, TABLE 5.1, TABLE 9, TABLE 9.1, TABLE 10, TABLE 10.1, TABLE 14, TABLE 14.1, TABLE 19, TABLE 20 and TABLE 30. In some embodiments, such a guide nucleic acid comprises a nucleotide sequence of at least about: 80%, 85%, 90%, 95%, 96%, 97%, 98%, or 99% sequence identity to any one of the sequences recited in TABLE 5, TABLE 5.1, TABLE 9, TABLE 9.1, TABLE 10, TABLE 10.1, TABLE 14, TABLE 14.1, TABLE 19, TABLE 20 and TABLE 30.

In some embodiments, a guide nucleic acid comprises a nucleotide sequence for targeting the effector protein to the B2M gene. In some embodiments, such a guide nucleic acid comprises a nucleotide sequence of any one of the sequences recited in TABLE 6, TABLE 6.1, TABLE 11, TABLE 11.1, TABLE 12, TABLE 12.1, TABLE 15, TABLE 15.1, TABLE 20 and TABLE 29. In some embodiments, such a guide nucleic acid comprises a nucleotide sequence of at least about: 80%, 85%, 90%, 95%, 96%, 97%, 98%, or 99% sequence identity to anyone of the sequences recited in TABLE 6, TABLE 6.1, TABLE 11, TABLE 11.1, TABLE 12, TABLE 12.1, TABLE 15, TABLE 15.1, TABLE 20 and TABLE 29.

In some embodiments, a guide nucleic acid comprises a nucleotide sequence for targeting the effector protein to the CIITA gene. In some embodiments, such a guide nucleic acid comprises a nucleotide sequence of any one of the sequences recited in TABLE 7, TABLE 7.1, TABLE 8, TABLE 13, TABLE 16 and TABLE 31. In some embodiments, such a guide nucleic acid comprises a nucleotide sequence of at least about: 80%, 85%, 90%, 95%, 96%, 97%, 98%, or 99% sequence identity to any one of the sequences recited in TABLE 7, TABLE 7.1, TABLE 8, TABLE 13, TABLE 16 and TABLE 31.

In some embodiments, a guide nucleic acid comprises shorter versions of the guide nucleic acids disclosed herein. For example, the guide nucleic acid sequence can consist of a portion of a guide nucleic acid disclosed herein. In some instances, shorter versions can provide enhanced activity relative to their longer versions. Examples of longer versions of guide RNA for CasΦ.12 are shown in TABLES 8, 9 and 11, whereas shorter versions are show in TABLES 14, 15 and 16. The shorter versions are produced by removing sixteen nucleotides from the 5′ end of the long version and three nucleotides from the 3′ end of the long version. In some embodiments, the long version is a CasΦ.32 guide nucleic acid described in TABLES 10, 12 and 13, and, similar to the guide RNA for CasΦ.12, the shorter version is a guide nucleic acid without the sixteen nucleotides at the 5′ end of the long version and without the three nucleotides at the 3′ end of the long version.

### Repeat Sequence

In some embodiments, the repeat region described herein comprises one or more 2′O-methyl modified nucleotides. In some embodiments, the repeat region described herein comprises at least one 2′O-methyl modified nucleotides. In some embodiments, the repeat region described herein comprises one, two, three, four or five 2′O-methyl modified nucleotides. In some embodiments, the repeat region described herein comprises one, two, three, four or five contiguous 2′O-methyl modified nucleotides. In some embodiments, 3′ end of any one of the repeat region described herein comprises one, two, three, four or five contiguous 2′O-methyl modified nucleotides.

In some embodiments, the repeat sequence of the guide nucleic acid comprises a hairpin. In some embodiments, the hairpin is in the 3′ portion of the repeat sequence. The hairpin comprises a double-stranded stem portion and a single-stranded loop portion. In some embodiments, one stand of the stem portion comprises a CYC sequence and the other strand comprises a GRG sequence, wherein Y and R are complementary. In some embodiments, the repeat sequence comprises a GAC sequence at the 3′ end. In some embodiments, the G of the GAC sequence is in the stem portion of the hairpin. In some embodiments, each strand of the stem portion comprises 3, 4, 5, 6, 7, 8, 9 or 10 nucleotides. In some embodiments, each strand of the stem portion comprises 3, 4 or 5 nucleotides. In some embodiments, the loop portion comprises 2, 3, 4, 5, 6, 7, 8, 9, 10 or more nucleotides. In some embodiments, the loop portion comprises 2, 3, 4, 5 or 6 nucleotides. In some embodiments, the loop portion comprises 4 nucleotides. In some embodiments, the nucleotides are naturally occurring nucleotides. In some embodiments, the nucleotides are synthetic nucleotides.

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence is connected to another sequence of a guide nucleic acid, such as an intermediary sequence, that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of any one of the repeat sequences in TABLE 2 and TABLE 3. In some embodiments, a repeat sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of any one of the sequences recited in TABLE 2 and TABLE 3.

### Spacer Sequence

In general, guide nucleic acids comprise a spacer region that hybridizes to a target sequence of a target nucleic acid, and a repeat region that interacts with (e.g., binds) the effector protein. The repeat region can also be referred to as a “protein-binding segment.” Typically, the repeat region is adjacent to the spacer region. For example, a guide nucleic acid that interacts (e.g., binds) with the effector protein comprises a repeat region that is 5′ of the spacer region. The spacer region of the guide nucleic acid can have complementarity with (e.g., hybridize to) an equal length portion of a target sequence of a target nucleic acid. In some embodiments, the spacer region is at least 90% identity, at least 91% identity, at least 92% identity, at least 93% identity, at least 94% identity, at least 95% identity, at least 96% identity, at least 97% identity, at least 98% identity, or at least 99% identity complementary to an equal length portion of a target sequence of the target nucleic acid. In some embodiments, the spacer region is 100% complementary to an equal length portion of a target sequence of a target nucleic acid. Alternatively, the spacer region of the guide nucleic acid can have a certain % identity to an equal length portion of a target sequence of a target nucleic acid. Accordingly, in some embodiments, the spacer region of the guide nucleic acid can have at least 90% identity, at least 910% identity, at least 92% identity, at least 93% identity, at least 94% identity, at least 95% identity, at least 96% identity, at least 97% identity, at least 98% identity, at least 99% identity, to an equal length portion of a target sequence of the target nucleic acid. In some embodiments, the spacer region is 100% identical to an equal length portion of a target sequence of a target nucleic acid.

In some embodiments, the spacer region described herein comprises one or more 2′O-methyl modified nucleotides. In some embodiments, the spacer region described herein comprises at least one 2′O-methyl modified nucleotides. In some embodiments, the spacer region described herein comprises one, two, three, four or five 2′O-methyl modified nucleotides. In some embodiments, the spacer region described herein comprises one, two, three, four or five contiguous 2′O-methyl modified nucleotides. In some embodiments, 5′ end of any one of the spacer region described herein comprises one, two, three, four or five contiguous 2′O-methyl modified nucleotides.

In some embodiments, the spacer region is 15-28 linked nucleotides in length. In some embodiments, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer region is 18-24 linked nucleotides in length. In some embodiments, the spacer region is at least 15 linked nucleotides in length. In some embodiments, the spacer region is at least 16, 18, 20, or 22 linked nucleotides in length. In some embodiments, the spacer region comprises at least 10, 11, 12, 13, 14, 15, 16, 17, 18, 19, 20, 21, 22, 23, 24, 25, 26, 27, 28, 29, or 30 nucleotides. In some embodiments, the spacer region is at least 17 linked nucleotides in length. In some embodiments, the spacer region is at least 18 linked nucleotides in length. In some embodiments, the spacer region is at least 20 linked nucleotides in length. In some embodiments, the spacer region comprises at least 15 contiguous nucleotides that are complementary to the target nucleic acid.

In some embodiments, the guide nucleic acid comprises a spacer sequence that is the same as or differs by no more than 5 nucleotides from a spacer sequence described herein (e.g., TABLES 5-16, 18-19, and 23) by no more than 4 nucleotides from a spacer sequence described herein (e.g., TABLES 5-16, 18-19, and 23), by no more than 3 nucleotides from a spacer sequence described herein (e.g., TABLES 5-16, 18-19, and 23), no more than 2 nucleotides from a spacer sequence described herein (e.g., TABLES 5-16, 18-19, and 23), or no more than 1 nucleotide from a spacer sequence described herein (e.g., TABLES 5-16, 18-19, and 23). A difference can be addition, deletion or substitution and where there are multiple differences, the differences can be addition, deletion and/or substitution. In the sequences provided in TABLES 8, 13 or 16, the base T is interchangeable with U when a guide nucleic either is or comprises ribonucleic or deoxyribonucleic nucleosides.

The spacer region of guide nucleic acids for the effector proteins disclosed herein can comprise a seed region. In some embodiments, the seed regions do not tolerate mismatches in the complementarity of a spacer and a target sequence within about 1 to about 20 nucleotides from the 5′ end of a spacer sequence. The seed region starts from the 5′ end of the spacer sequence and is a region in which mismatches in the complementarity between the spacer sequence and the target sequence are not tolerated when the guide nucleic acid is bound to an effector protein such that the guide nucleic acid does not hybridize to the target sequence to allow cleavage of the target nucleic acid by the effector protein. In some embodiments, the seed region comprises between 10 and 20 nucleotides, between 12 and 20 nucleotides, between 14 and 20 nucleotides, between 14 and 18 nucleotides, between 10 and 16 nucleotides, between 12 and 16 nucleotides, or between 14 and 16 nucleotides. In some embodiments, the seed region comprises 16 nucleotides.

### Linker for Nucleic Acids

In some embodiments, guide nucleic acids comprise one or more linkers connecting different nucleotide sequences as described herein. A linker may comprise 1, 2, 3, 4, 5, 6, 7, 8, 9, 10, or more nucleotides. In some embodiments, the guide nucleic acid comprises at least one, at least two, at least three, at least four, at least five, at least six, at least seven, at least eight, at least nine, or at least ten linkers. In some embodiments, the guide nucleic acid comprises more than one linker. In some embodiments, at least two of the more than one linker are the same. In some embodiments, at least two of the more than one linker are not same. In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

A linker may be any suitable linker, examples of which are described herein. In some embodiments, a linker comprises a nucleotide sequence of 5′-GAAA-3′.

### Intermediary Sequence

Guide nucleic acids described herein may comprise one or more intermediary sequences. In general, an intermediary sequence used in the present disclosure is not transactivated or transactivating. An intermediary sequence may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop structure linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

In some embodiments, an intermediary sequence comprises a nucleotide sequence that is at least 65%, at least 70%, at least 75%, at least 80%, at least 85%, at least 90%, at least 95%, at least 97%, or at least 98%, at least 99%, or 100% identical to any one of the intermediary sequences in TABLE 4. In some embodiments, an intermediary sequence comprises at least 5, at least 6, at least 7, at least 8, at least 9, at least 10, at least 20, at least 30, at least 40, at least 50, at least 60, at least 70, at least 80, at least 90, at least 100, at least 110, at least 120, at least 130, or at least 140 contiguous nucleotides of any one of the intermediary sequences recited in TABLE 4.

### Handle Sequence

Guide nucleic acids described herein may comprise one or more handle sequences. In some embodiments, the handle sequence comprises an intermediary sequence. In such instances, at least a portion of an intermediary sequence non-covalently bonds with an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such instances, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided herein. In some embodiments, the linker comprises a sequence of 5′-GAAA-3′. In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

A handle sequence may comprise or form a secondary structure (e.g., one or more hairpin loops) that facilitates the binding of an effector protein to a guide nucleic acid and/or modification activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). An effector protein may recognize a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

### A Single Nucleic Acid System

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR1) of the guide nucleic acid non-covalently interacts with the one or more polypeptides described herein. In some embodiments, a second region (FR2) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA. crRNA

In some embodiments, a guide nucleic acid comprises a crRNA. In some embodiments, the guide nucleic acid is the crRNA. In general, a crRNA comprises a first region (FR1) and a second region (FR2), wherein the FR1 of the crRNA comprises a repeat sequence, and the FR2 of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary sequence. In some embodiments, a single nucleic acid system comprises a guide nucleic acid comprising a crRNA and an intermediary sequence.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

In some embodiments, a crRNA comprises a nucleotide sequence that is at least 65%, at least 70%, at least 75%, at least 80%, at least 85%, at least 90%, at least 95%, at least 97%, or at least 98%, at least 99%, or 100% identical to any one of the crRNA sequences in TABLE 8, TABLE 9, TABLE 9.1, TABLE 10, TABLE 10.1, TABLE 11, TABLE 11.1, TABLE 12, TABLE 12.1, TABLE 13, TABLE 14, TABLE 14.1, TABLE 15, TABLE 15.1, TABLE 16, TABLE 18 and TABLE 25. In some embodiments, a crRNA sequence comprises a repeat sequence that is at least 65%, at least 70%, at least 75%, at least 80%, at least 85%, at least 90%, at least 95%, at least 97%, or at least 98%, at least 99%, or 100% identical to any one of the sequences set forth in TABLE 2 and TABLE 3, and a spacer sequence that is at least 65%, at least 70%, at least 75%, at least 80%, at least 85%, at least 90%, at least 95%, at least 97%, or at least 98%, at least 99%, or 100% identical to any one of the sequences set forth in TABLE 5-16, 18-19, and 23. In some embodiments, a crRNA comprises at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 contiguous nucleotides of any one of the crRNA sequences recited in TABLE 8, TABLE 9, TABLE 9.1, TABLE 10, TABLE 10.1, TABLE 11, TABLE 11.1, TABLE 12, TABLE 12.1, TABLE 13, TABLE 14, TABLE 14.1, TABLE 15, TABLE 15.1, TABLE 16, TABLE 18 and TABLE 25. In some embodiments, a crRNA sequence comprises at least 5, at least 6, at least 7, at least 8, at least 9, or at least 10 contiguous nucleotides of any one of the repeat sequences recited in TABLE 2 and TABLE 3, and at least 5, at least 6, at least 7, at least 8, at least 9, or at least 10 contiguous nucleotides of any one of the spacer sequences recited in TABLE 5-16, 18-19, and 23.

TABLE 2 and TABLE 3 provide illustrative crRNA sequences for use with the viral vectors and methods described herein. In some embodiments, the crRNA of TABLE 2 and TABLE 3 can be combined with the spacer sequences described herein, for targeting an effector protein described herein to the TRAC gene, the B2M gene or the CIITA gene. In some embodiments, the crRNA sequence comprises at least 70%, at least 80%, at least 90%, at least 92%, at least 95%, at least 97%, or at least 99%, or 100% sequence identity to any one of SEQ ID NO: 204-226, or a complement thereof. In some embodiments, the crRNA comprises a nucleotide sequence of any one of SEQ ID NO: 1588-1625 as shown in TABLE 3. In some embodiments, the nucleotide sequence of the crRNA is at least 65%, at least 70%, at least 75%, at least 80%, at least 85%, at least 90%, at least 95%, or 100% identical to any one of SEQ ID NO: 1588-1625. sgRNA

In some embodiments, a guide nucleic acid comprises a sgRNA. In some embodiments, a guide nucleic acid is a sgRNA. In some embodiments, a sgRNA comprises a first region (FR1) and a second region (FR2), wherein the FR1 comprises a handle sequence and the FR2 comprises a spacer sequence. In some embodiments, the handle sequence and the spacer sequences are directly connected to each other (e.g., covalent bond (phosphodiester bond)). In some embodiments, the handle sequence and the spacer sequence are connected by a linker.

In some embodiments, a sgRNA comprises one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g, covalently linked, such as through a phosphodiester bond) In some embodiments, a repeat sequence and a spacer sequence are linked in an sgRNA by any suitable linker, examples of which are provided herein.

In some embodiments, a sgRNA comprises a nucleotide sequence that is at least 65%, at least 70%, at least 75%, at least 80%, at least 85%, at least 90%, at least 95%, at least 97%, or at least 98%, at least 99%, or 100% identical to any one of the sequences recited in TABLE 17,26 and 36. In a single nucleic acid system, any one of the sequences recited in TABLE 3 can be combined with any one of the sequences recited in TABLE 4 to form a handle sequence, wherein the handle sequence upon combining with the spacer sequences described herein forms a sgRNA. For example, in some embodiments, the crRNA and tracrRNA sequence of TABLE 3 and TABLES 4 can be combined to form sgRNA, when combined with the spacer sequences described herein, for targeting an effector protein described herein to the TRAC gene, the B2M gene or the CIITA gene. In such embodiments, the tracrRNA sequence comprises a nucleotide sequence of any one of SEQ ID NO: 385-440 as shown in TABLE 4. In some embodiments, the nucleotide sequence of the tracrRNA sequence is at least 65%, at least 70%, at least 75%, at least 80%, at least 85%, at least 90%, at least 95%, or 100% identical to any one of SEQ ID NO: 385-440.

### A Dual Nucleic Acid System

In a dual nucleic acid system, an effector protein is enabled to have a binding and/or nuclease activity on a target nucleic acid, by a tracrRNA or a tracrRNA-crRNA duplex. In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, activity of effector protein requires binding to a tracrRNA molecule. In some embodiments, the dual nucleic acid system comprises a guide nucleic acid and a tracrRNA, wherein the tracrRNA is an additional nucleic acid capable of at least partially hybridizing to the first region of the guide nucleic acid. In some embodiments, the tracrRNA or additional nucleic acid is capable of at least partially hybridizing to the 5′ end of the second region of the guide nucleic acid.

The tracrRNA can comprise deoxyribonucleosides in addition to ribonucleosides. The tracrRNA can be separate from but form a complex with a guide nucleic acid. In some embodiments, the guide nucleic acid and the tracrRNA are separate polynucleotides. A tracrRNA can comprise a repeat hybridization region and a hairpin region. The repeat hybridization region can hybridize to all or part of the sequence of the repeat of a guide nucleic acid. The repeat hybridization region can be positioned 3′ of the hairpin region. The hairpin region can comprise a first sequence, a second sequence that is reverse complementary to the first sequence, and a stem-loop linking the first sequence and the second sequence.

In some embodiments, the length of the tracrRNA is not greater than 50, 56, 68, 71, 73, 95, or 105 linked nucleotides. In some embodiments, the length of a tracrRNA is about 30 to about 120 linked nucleotides. In some embodiments, the length of a tracrRNA is about 50 to about 105, about 50 to about 95, about 50 to about 73, about 50 to about 71, about 50 to about 68, or about 50 to about 56 linked nucleotides. In some embodiments, the length of a tracrRNA is 56 to 105 linked nucleotides, from 56 to 105 linked nucleotides, 68 to 105 linked nucleotides, 71 to 105 linked nucleotides, 73 to 105 linked nucleotides, or 95 to 105 linked nucleotides. In some embodiments, the length of a tracrRNA is 40 to 60 nucleotides. In some embodiments, the length of the tracrRNA is 50, 56, 68, 71, 73, 95, or 105 linked nucleotides. In some embodiments, the length of the tracrRNA is 50 nucleotides.

An exemplary tracrRNA can comprise, from 5′ to 3′, a 5′ region, a hairpin region, a repeat hybridization region, and a 3′ region. In some embodiments, the 5′ region can hybridize to the 3′ region. In some embodiments, the 5′ region does not hybridize to the 3′ region. In some embodiments, the 3′ region is covalently linked to the guide nucleic acid (e.g., through a phosphodiester bond). In some embodiments, a tracrRNA can comprise an unhybridized region at the 3′ end of the tracrRNA. The unhybridized region can have a length of about 1, about 2, about 3, about 4, about 5, about 6, about 7, about 8, about 9, about 10, about 12, about 14, about 16, about 18, or about 20 linked nucleotides. In some embodiments, the length of the un-hybridized region is 0 to 20 linked nucleotides.

In some embodiments, the guide nucleic acid does not comprise a tracrRNA. In some embodiments, an effector protein does not require a tracrRNA to locate and/or cleave a target nucleic acid. In some embodiments, the guide nucleic acid comprises a repeat region and a spacer region, wherein the repeat region binds to the effector protein and the spacer region hybridizes to a target sequence of the target nucleic acid. The repeat sequence of the guide nucleic acid can interact with an effector protein, allowing for the guide nucleic acid and the effector protein to form an RNP complex.

TABLE 3 and TABLES 4 provides exemplary combination comprising effector proteins, crRNAs (repeat sequence), and tracrRNAs. Each row in TABLE 3 and TABLES 4 represents an exemplary combination. Moreover, in a dual nucleic acid system, a tracrRNA comprising any one of the nucleotide sequence recited in TABLE 4, and a guide RNA comprising any one of repeat sequence of the crRNA recited in TABLE 3 can be combined with the spacer sequences described herein for targeting an effector protein described herein to the TRAC gene, the B2M gene or the CIITA gene. In such embodiments, the tracrRNA comprises a nucleotide sequence of any one of SEQ ID NO: 385-440 as shown in TABLE 4. In some embodiments, the nucleotide sequence of the tracrRNA is at least 65%, at least 70%, at least 75%, at least 80%, at least 85%, at least 90%, at least 95%, or 100% identical to any one of SEQ ID NO: 385-440.

### Donor Nucleic Acid

In some embodiments, viral vectors provided herein comprise a nucleotide sequence that comprises a donor nucleic acid, wherein the donor nucleic acid encodes a CAR. Introduction of such a donor nucleic acid into a T cell, as described herein, generates a “CAR T cell.” In general, a CAR comprises an antigen binding domain that is expressed on the surface of the CAR T-cell. The antigen binding domain can be considered to be an extracellular domain. In general, the antigen binding domain binds an antigen on a target cell. The antigen binding domain can comprise an antibody. The antibody can comprise an immunoglobulin or antigen binding fragment thereof. The antibody can be a polyclonal antibody or a monoclonal antibody. The antigen binding domain can comprise or consist essentially of an antigen binding antibody fragment, referred to simply herein as an antibody fragment. Non-limiting examples of antibody fragments include Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), and isolated CDRs.

In some embodiments, the antigen binding portion of the CAR binds to an antigen that is specific to a pathogen. In some embodiments, the antigen binding portion of the CAR recognizes an antigen expressed on the surface of the infected cell due to the infection/pathogen (e.g., hepatitis virus, human immunodeficiency virus, influenza virus and corona virus).

In some embodiments, the antigen binding portion of the CAR binds an antigen expressed by a cancer cell. Such an antigen expressed by a cancer cell can be a result of the cell harboring one or more mutations that results in unchecked proliferation of the cancer cell. In some embodiments, the antigen expressed by a cancer cell is selected from the group consisting of ADRB3, AKAP-4,ALK, Androgen receptor, B7H3, BCMA, BORIS, BST2, CAIX, CD 179a, CD123, CD171, CD19, CD20, CD22, CD24, CD30, CD300LF, CD33, CD38, CD44v6, CD72, CD79a, CD79b, CD97, CEA, CLDN6, CLEC12A, CLL-1, CS-1, CXORF61, CYP1B1, Cyclin B 1, E7, EGFR, EGFRvIII, ELF2M, EMR2, EPCAM, ERBB2 (Her2/neu), ERG (TMPRSS2 ETS fusion gene), ETV6-AML, EphA2, Ephrin B2, FAP, FCAR, FCRL5, FLT3, Folate receptor alpha, Folate receptor beta, Fos-related antigen 1, Fucosyl GMl, GD2, GD3, GM3, GPC3, GPR20, GPRC5D, GloboH, HAVCR1, HMWMAA, HPV E6, IGF-I receptor, IL-13Ra2, IL-11Ra, KIT, LAGE-1a, LAIR1, LCK, LILRA2, LMP2, LY6K, LY75, LewisY, MAD-CT-1, MAD-CT-2, MAGE A1, MAGE-A1, ML-IAP, MUC1, MYCN, MelanA/MARTl, Mesothelin, NA17, NCAM, NY-BR-1, NY-ESO-1, OR51E2, OY- TES 1, PANX3, PAP, PAX3, PAX5, PCTA-1/Galectin 8, PDGFR-beta, PLAC1, PRSS21, PSCA, PSMA, Polysialic acid, Prostase, RAGE-1, ROR1, RU1, RU2, Ras mutant, RhoC, SART3, SSEA-4, SSX2, TAG72, TARP, TEM1/CD248, TEM7R, TGS5, TRP-2, TSHR, Tie 2, Tn Ag, UPK2, VEGFR2, WT1, XAGE1, and IGLL1.

In some embodiments, the donor nucleic acid includes, in addition to the nucleotide sequence encoding a CAR, one or more nucleotide sequences for directing integration of the donor nucleic acid into the TRAC gene of the target cell (e.g., T cell). These one or more nucleotide sequences can be used by the molecular machinery (homologous recombination (e.g., homology directed repair (HDR)) or non-homologous end joining (NHEJ)) present in the target cell (either naturally present or recombinantly introduced) for directing integration of the donor nucleic acid into the TRAC gene. In some embodiments, a donor nucleic acid comprises one nucleotide sequence to one side (5′ or 3′) of the nucleotide sequence encoding a CAR, such that integration of the donor nucleic acid is selective for the TRAC gene of the target cell. In some embodiments, such nucleotide sequences are located on both sides (5′ and 3′) of the nucleotide sequence encoding a CAR.

In some embodiments, the one or more nucleotide sequences for directing integration of the donor nucleic acid into the TRAC gene are identical or complementary to a target sequence in the TRAC gene. Exemplary lengths of identity or complementarity between the TRAC gene and the nucleotide sequence for directing integration include at least 5 nucleotides, at least 10 nucleotides, at least 15 nucleotides, at least 20 nucleotides, at least 25 nucleotides, or at least 30 nucleotides. In some embodiments, the length of identity or complementarity is no more than about 30, no more than about 40, or no more than about 50 nucleotides. In some embodiments, the one or more nucleotide sequences for directing integration share identity or complementarity with a target sequence in the TRAC gene that is about 5 nucleotides to about 50 nucleotides, about 10 nucleotides to about 50 nucleotides, about 15 nucleotides to about 50 nucleotides, about 20 nucleotides to about 50 nucleotides, about 25 nucleotides to about 50 nucleotides, about 30 nucleotides to about 50 nucleotides, about 5 nucleotides to about 40 nucleotides, about 10 nucleotides to about 40 nucleotides, about 15 nucleotides to about 40 nucleotides, about 20 nucleotides to about 40 nucleotides, about 25 nucleotides to about 40 nucleotides, about 30 nucleotides to about 40 nucleotides, about 5 nucleotides to about 30 nucleotides, about 10 nucleotides to about 30 nucleotides, about 15 nucleotides to about 30 nucleotides, about 20 nucleotides to about 30 nucleotides, about 5 nucleotides to about 25 nucleotides, about 10 nucleotides to about 25 nucleotides, about 15 nucleotides to about 25 nucleotides, about 20 nucleotides to about 25 nucleotides, about 5 nucleotides to about 20 nucleotides, about 10 nucleotides to about 20 nucleotides, about 15 nucleotides to about 20 nucleotides, about 5 nucleotides to about 15 nucleotides, about 10 nucleotides to about 15 nucleotides, or about 5 nucleotides to about 10 nucleotides in length.

In general, a CAR comprises an intracellular binding domain. The intracellular binding domain generally contributes to the activation of the CAR T-cell when the antigen binding domain of the CAR associates with its respective antigen. In some embodiments, the intracellular signaling domain of said CAR comprises a functional signaling domain of a protein selected from the group consisting of 4-1BB (CD137), B7-H3, BAFFR, BLAME (SLAMF8), CD100 (SEMA4D), CD103, CD150, CD160, CD160 (BY55), CD162 (SELPLG), CD18, CD19, CD2, CD229, CD27, CD28, CD29, CD30, CD4, CD40, CD49D, CD49a, CD49f, CD69, CD7, CD84, CD8alpha, CD8beta, CD96, CDS, CD11a, CD11b, CD11c, CD11d, CEACAM1, CRTAM, DNAM1 (CD226), GADS, GITR, HVEM (LIGHTR), IA4, ICAM-1, ICOS, IL2R beta, IL2R gamma, IL7R alpha, ITGA4, ITGA4, ITGA6, ITGAD, ITGAE, ITGAL, ITGAM, ITGAX, ITGB 1, ITGB2, ITGB7, LAT, LFA-1, LFA-1, LIGHT, LTBR, NKG2C, NKp30, NKp44, NKp46, NKp80 (KLRF1), OX40, PAG/Cbp, PD-1, PSGL1, SLAMF1, SLAMF4, SLAMF6, SLAMF7, SLP-76, TNFR2, TRANCE/RANKL, VLA1, and VLA-6.

In some embodiments, the donor nucleic acid encoding the CAR has a length of about 500 nucleotides to about 1,000 nucleotides, about 1,000 nucleotides to about 1,500 nucleotides, about 1,500 nucleotides to about 2,000 nucleotides, or about 2,000 nucleotides to about 2,500 nucleotides. In some embodiments, the donor nucleic acid has a length of about 1,000 nucleotides to about 2,000 nucleotides. In some embodiments, the length of the donor nucleic acid is about 2,000 nucleotides to about 2,500 nucleotides. In some embodiments, the length of the donor nucleic acid is about 1,000 nucleotides to about 1,200 nucleotides, about 1,200 nucleotides to about 1,600 nucleotides, about 1,600 nucleotides to about 2,000 nucleotides, about 1,200 nucleotides to about 1,400 nucleotides, about 1,400 nucleotides to about 1,600 nucleotides, about 1,600 nucleotides to about 1,800 nucleotides, about 1,800 nucleotides to about 2,000 nucleotides.

In some embodiments, the donor nucleic acid of a viral vector described herein includes a sequence of nucleotides that will be or has been introduced into a cell following introduction of the viral vector. The donor nucleic acid can be introduced into the cell by any mechanism, including transfecting or transducing the viral vector. The viral vector, once introduced into the cell, can be integrated into the genome of the cell or remain as an episomal plasmid or viral genome. When used in reference to the activity of an effector protein, the donor nucleic acid includes a sequence of nucleotides that will be or has been inserted at the site of cleavage by the effector protein. When used in reference to homologous recombination, the donor nucleic acid can be a sequence of DNA that serves as a template in the process of homologous recombination, which can carry the modification that is to be or has been introduced into the target nucleic acid. By using this donor nucleic acid as a template, the genetic information, including the modification, is copied into the target nucleic acid by way of homologous recombination.

### Pharmaceutical Compositions

Disclosed herein, in some aspects, are pharmaceutical composition comprising a vector (e.g., a non-viral vector comprising a sequence encoding the genome editing tools described herein; a viral vector or a viral particle comprising a viral vector, wherein the viral vector comprises a sequence encoding the genome editing tools described herein); and a pharmaceutically acceptable excipient, carrier or diluent. Non-limiting examples of pharmaceutically acceptable excipients, carriers and diluents include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); and preservatives.

In some aspects, also provided herein is a pharmaceutical composition comprising CAR T cell or a population of CAR T cells as described herein; and a pharmaceutically acceptable excipient, carrier or diluent. Such an excipient, carrier or diluent, in this context, include those that facilitate storage of the cells in a freezer, such a dimethyl sulfoxide, HSA and alternative solvents/excipients as cryopreservation agents, and other excipients, such as sodium chloride, dextrose, dextran 40, electrolytes (e.g., Plasma-Lyte A), polyampholytes (e.g., methacrylates or poly-lysine), pore-forming amphipathic pH-responsive polymers facilitating the intracellular entry of non-reducing cryoprotectant sugars (e.g., comb-like pseudopeptides harbouring alkyl side chains that mimic fusogenic proteins), dimethyl sulfoxide, 1,2-propanediol, glycerol, sorbitol, poly(ethylene glycol) 600, trehalose, creatin, isoleucine, maltose, and sucrose, including those described by van der Walle et al., (2021), Pharmaceutics 13:1317, and Sheskey et al., Handbook of Pharmaceutical Excipients, 9th ed., Pharmaceutical Press: London, U K, 2020.

### Methods of Producing CAR T Cells

Provided herein are methods of producing an immunologically compatible CAR T cell or a population of such cells. In general, the compositions (e.g., viral vectors, viral particles, pharmaceutical composition, RNP complexes of effector proteins and guide nucleic acids) and systems disclosed herein can be used to produce an immunologically compatible CAR T cell or a population of such cells. Use of such effector proteins, multimeric complexes thereof and systems described herein can provide for modifying a target nucleic acid (e.g., the TRAC gene, the B2M gene and the CIITA gene) present in the starting T cell by the generation of a mutation (e.g., indel) into the target nucleic acid. Additionally, in the context of a donor nucleic acid, such compositions (e.g., viral vectors, viral particles, non-viral vectors, pharmaceutical composition, RNP complexes of effector proteins and guide nucleic acids) and systems can be used to specifically introduce the donor nucleic acid encoding a CAR into the TRAC gene of a starting T cell, thereby generating a CAR T cell. The generation of a mutation (e.g., indel) into a target nucleic acid (e.g., B2M gene and/or CIITA gene) and introduction of the donor nucleic acid into the TRAC gene can comprise one or more effector protein cleaving the target nucleic acid, thereby leading to deletion of one or more nucleotides of the target nucleic acid and/or insertion one or more nucleotides into the target nucleic acid (e.g., inserting the donor nucleic acid encoding a CAR), or otherwise mutating one or more nucleotides of the target nucleic acid, which leads to preventing the expression (e.g., gene silencing or removal of all expression (knock out)) of the protein, polypeptide or peptide encoded by the target nucleic acid (e.g., T-cell receptor alpha-constant, beta-2 microglobulin, and/or class II major histocompatibility complex transactivator). Such mutations lead to production of an immunologically compatible CAR T cell. Moreover, the methods provided herein have a particular advantage to the methods known in the art for generating a CAR T cell, in that the methods provided herein provide for the generation of an immunologically compatible CAR T cell in a rapid and cost effective fashion by use of one or two contacting steps with the compositions (e.g., viral vectors, viral particles, non-viral vectors, pharmaceutical composition, RNP complexes of effector proteins and guide nucleic acids) disclosed herein followed by a single culturing step for generation of the CAR T immunologically compatible CAR T cell. Such methods require no other agent that alters the CAR T-cell's ability to recognize a target cell or pathogen or autoreactivity of the CAR T-cell in a subject.

Accordingly, in some aspects, provided herein is a method of producing an immunologically compatible CAR T cell comprising: contacting ex vivo a T cell with a viral vector described herein, a viral particle described herein, or the pharmaceutical composition comprising a viral vector or a viral particle described herein for a sufficient period of time to allow for viral transduction of the T cell; and culturing the T cell for a sufficient period of time for indels to occur in the TRAC gene, B2M gene and CIITA gene and for integration of the donor nucleic acid into the TRAC gene, thereby producing the immunologically compatible CAR T cell. Similarly, also provided herein, in some aspects, is a method of producing a population of immunologically compatible CAR T cells comprising: contacting ex vivo a population of T cells with a viral vector described herein, a viral particle described herein, or the pharmaceutical composition comprising a viral vector or a viral particle described herein for a sufficient period of time to allow for viral transduction of T cells contained in the population; and culturing the population of T cells for sufficient period of time for indels to occur in the TRAC gene, B2M gene and CIITA gene and for integration of the donor nucleic acid into the TRAC gene, thereby producing the population of immunologically compatible CAR T cells.

Also provided herein is a method of producing an immunologically compatible CAR T cell comprising: contacting ex vivo a T cell with a viral vector or viral particle comprising a donor nucleic acid encoding the CAR for a sufficient period of time to allow for viral transduction of the T cell; contacting ex vivo the T cell with at least three different RNP complexes comprising an effector protein and a guide nucleic acid as described herein for targeting the effector protein to the TRAC gene, B2M gene and CIITA gene; and culturing the T cell for a sufficient period of time for indels to occur in the TRAC gene, B2M gene and CIITA gene and for integration of the donor nucleic acid into the TRAC gene, thereby producing the immunologically compatible chimeric antigen receptor (CAR) T cell. Similarly, also provided herein, in some aspects, is a method of producing a population of immunologically compatible CAR T cells comprising: contacting ex vivo a population of T cells with a viral vector or viral particle comprising a donor nucleic acid encoding the CAR for a sufficient period of time to allow for viral transduction of T cells contained in the population; contacting ex vivo the population of T cells with at least three different RNP complexes as described herein for targeting the effector protein to the TRAC gene, B2M gene and CIITA gene; and culturing the population of T cells for sufficient period of time for indels to occur in the TRAC gene, B2M gene and CIITA gene and for integration of the donor nucleic acid into the TRAC gene, thereby producing the population of chimeric antigen receptor (CAR) T cells.

In some embodiments, an RNP used in the above method comprises an effector protein and a guide nucleic acid as described herein. For example, in some embodiments, the effector protein is an effector protein described herein and the guide nucleic acid comprises a sequence that is at least 90% identical or complementary to an equal length portion of a target sequence of a TRAC gene. In some embodiments, the effector protein is an effector protein described herein and the guide nucleic acid comprises a sequence that is at least 90% identical or complementary to an equal length portion of a target sequence of a B2M gene. In some embodiments, the effector protein is an effector protein described herein and the guide nucleic acid comprises a sequence that is at least 90% identical or complementary to an equal length portion of a target sequence of a CIITA gene. In some embodiments, contacting ex vivo the T cell with the RNP complexes described herein include electroporation, lipofection, or lipid nanoparticle (LNP) delivery of the RNP complexes to the T cell(s).

In some embodiments, the methods provided herein include contacting the T cells ex vivo with a viral vector described herein, a viral particle described herein, a non-viral vector described herein or the pharmaceutical composition comprising a viral vector, a viral particle, or a non-viral vector described herein for a specified period of time that allows for the transduction of the T cell(s). In some embodiments, such contacting comprises at least about 1 hour, at least about 2 hours, at least about 3 hours, at least about 4 hours, at least about 5 hours or at least about 6 hours. Such contacting can also be limited to a specific period of time, such as the contacting being no more than 10 hours, no more than 9 hours, no more than 8 hours, no more than 7, hours, no more than 6 hours, no more than 5 hours, no more than 4 hours, no more than 3 hours or no more than 2 hours. Accordingly, the period for contacting can be for about 1 hour to about 10 hours, about 1 hour to about 9 hours, about 1 hour to about 8 hours, about 1 hour to about 7 hours, about 1 hour to about 6 hours, about 1 hour to about 5 hours, about 1 hour to about 4 hours, about 1 hour to about 3 hours, about 1 hour to about 2 hours, about 2 hour to about 10 hours, about 2 hour to about 9 hours, about 2 hour to about 8 hours, about 2 hour to about 7 hours, about 2 hour to about 6 hours, about 2 hour to about 5 hours, about 2 hour to about 2 hours, or about 2 hour to about 3 hours.

The ex vivo contacting of the T cell or T cell population with a viral vector described herein, a viral particle described herein, a non-viral vector described herein, or the pharmaceutical composition comprising a viral vector, a viral particle, or a non-viral vector described herein can be performed using methods described herein (e.g., Example 14) or a method well known in the art, such as the methods described by Viney et al., (2021) and J Virol., 95(7):e02023-20, Nawaz, et al., (2021), Blood Cancer J., 11:119, each of which is incorporated by reference in its entirety.

Methods of introducing a nucleic acid and/or protein into a host cell (e.g., T cell) are known in the art, and any convenient method may be used to introduce a subject nucleic acid (e.g., an expression construct/vector) into a target cell (e.g., T cell). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like. In some embodiments, molecules of interest, such as nucleic acids of interest, are introduced to T cells. In some embodiments, an effector protein is introduced to T cells. In some embodiments, vectors, such as lipid particles and/or viral vectors may be introduced to T cells. Introduction may be for contact with a host or for assimilation into the host, for example, introduction into T cells.

In some embodiments, an effector protein may be provided as RNA. The RNA may be provided by direct chemical synthesis or may be transcribed in vitro from a DNA (e.g., encoding the effector protein). Once synthesized, the RNA may be introduced into T cells by way of any suitable technique for introducing nucleic acids into cells (e.g., microinjection, electroporation, transfection, etc.). In some embodiments, introduction of one or more nucleic acid may be through the use of a vector and/or a vector system, accordingly, in some embodiments, compositions and system described herein comprise a vector and/or a vector system.

Vectors may be introduced directly to T cells. In some embodiments, T cells may be contacted with one or more vectors as described herein, and in some embodiments, said vectors are taken up by the cells. Methods for contacting T cells with vectors include but are not limited to electroporation, calcium chloride transfection, microinjection, lipofection, micro-injection, contact with the T cells or particle that comprises a molecule of interest, or a package of T cells or particles that comprise molecules of interest.

Components described herein may also be introduced directly to T cells. For example, an engineered guide nucleic acid may be introduced to T cells, specifically introduced into T cells. Methods of introducing nucleic acids, such as RNA into T cells include, but are not limited to direct injection, transfection, or any other method used for the introduction of nucleic acids.

In some embodiments, the methods provided herein include contacting the T cells ex vivo with a specific amount of viral vector or viral particles. In general, the amount of viral vector or vial particles is identified in reference to the number of cells that are present in the culturing containing the T cells, termed a multiplicity of infection (MOI). Accordingly, in some embodiments, the method provided herein comprises using an MOI of viral vector or viral particle to T cell of about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, about 1×10⁹, about 5×10⁹, about 1×10¹⁰, or about 5×10¹⁰. In some embodiments, the MOI is about 1×10⁴. In some embodiments, the MOI is about about 5×10⁴. In some embodiments, the MOI is about 1×10⁵. In some embodiments, the MOI is about 5×10⁴. In some embodiments, the MOI is about 1×10⁶. In some embodiments, the MOI is about 5×10⁶. In some embodiments, the MOI is about 1×10⁷. In some embodiments, the MOI is about 5×10⁷. In some embodiments, the MOI is about 1×10⁸. In some embodiments, the MOI is about 5×10⁸. In some embodiments, the MOI is about 1×10⁹. In some embodiments, the MOI is about 5×10⁹. In some embodiments, the MOI is about 1×10¹⁰. In some embodiments, the MOI is about 5×10¹⁰.

In some embodiments, the methods provided herein, once completed with the contacting step(s), are cultured for a period of time sufficient for the effector protein, guide nucleic acids and donor nucleic acid to generate indels in the TRAC gene, B2M gene, and CIITA gene and for integration of the donor nucleic acid into the TRAC gene. Accordingly, in some embodiments, the culturing is for at least 1 day, at least 2 days, at least 3 days, at least 4 days, at least 5 days, or at least 6 days. Such culturing can also be limited to a specific period of time, such as the culturing being no more than 7 days, no more than 8 days, no more than 9 days, no more than 10 days, no more than 11 days, no more than 12 days, no more than 13 days, no more than 14 days, no more than 15 days, no more than 16 days, no more than 17 days, no more than 18 days, no more than 19 days, no more than 20 days, or no more than 21 days.

In some embodiments, the methods provided herein for generating a population of T cells includes a period of time for culturing the T cells such that a certain percentage of T cells include mutations (e.g., indels) in the TRAC gene, B2M gene and CIITA gene and integration of the donor nucleic acid into the TRAC gene. Accordingly, in some embodiments, the period of time is sufficient for at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76% at least 77%, at least 78%, at least 79%, at least 80% of the T cells contained in the population to have mutations (e.g., indels) occur in the TRAC gene, B2M gene and CIITA gene and integration of the donor nucleic acid. In some embodiments, the period of time is sufficient for at least 50% of the T cells contained in the population to have indels occur in the TRAC gene, B2M gene and CIITA gene and integration of the donor nucleic acid. In some embodiments, the period of time is sufficient for at least 55% of the T cells contained in the population to have indels occur in the TRAC gene, B2M gene and CIITA gene and integration of the donor nucleic acid. In some embodiments, the period of time is sufficient for at least 60% of the T cells contained in the population to have indels occur in the TRAC gene, B2M gene and CIITA gene and integration of the donor nucleic acid. In some embodiments, the period of time is sufficient for at least 65% of the T cells contained in the population to have indels occur in the TRAC gene, B2M gene and CIITA gene and integration of the donor nucleic acid. In some embodiments, the period of time is sufficient for at least 75% of the T cells contained in the population to have indels occur in the TRAC gene, B2M gene and CIITA gene and integration of the donor nucleic acid. In some embodiments, the period of time is sufficient for at least 80% of the T cells contained in the population to have indels occur in of TRAC gene, B2M gene and CIITA gene and integration of the donor nucleic acid.

Methods for assessing the number of cells in the population having the specified mutations include the methods described herein (e.g., Example 14) or any other method well known in the art, such as sequencing, use of photocleavable guide RNAs, and qPCR as further described by Zou et al., (2021) STAR Protoc., 2(4):100909 and Li et al., (2019), Sci Rep, 9:18877, each of which is incorporated by reference in its entirety.

In some embodiments, the methods provided herein end with the freezing the CAR T cell or CAR T cell population. Such freezing provides for the long term storage of the CAR T cell or CAR T cell population and future use. Freezing of the CAR T cell or CAR T cell population can be performed using methods well known in the art for preserving the cells, especially T cells, including the addition of cryoprotectants for preserving post-thaw proliferative capacity, phenotype and functional response. Exemplary cryoprotectants and methods for preserving such functions are described in Luo et al., (2017), Cryobiology. 79:65-70, which is incorporated by reference in its entirety.

Because of the limited number of contacting and culturing steps that are required by the methods provided herein, the number of T cells that are killed are greatly reduced compared to other methods known in the art. Accordingly, in some embodiments, the number of T cells that are killed during the method is no more than 1%, 2%, 3%, 4%, 5%, 6%, 7%, 8%, 9%, 10%, 11%, 12%, 13%, 14%, 15% based on the number of T cells present in the population at the start of the method. In some embodiments, the number of cells killed is less than 1%. In some embodiments, the number of T cells that are killed is no more than 3%. In some embodiments, the number of T cells that are killed is no more than 5%. In some embodiments, the number of T cells that are killed is no more than 10%. In some embodiments, the number of T cells that are killed is no more than 15%.

In some embodiments, effector protein mediated cleavage (single-stranded or double-stranded) is site-specific, meaning cleavage occurs at a specific site in the target nucleic acid, often within the region of the target nucleic acid that hybridizes with the guide nucleic acid spacer sequence. In some embodiments, the effector proteins introduce a single-stranded break in a target nucleic acid to produce a cleaved nucleic acid. In some embodiments, the effector protein is capable of introducing a break in a single stranded RNA (ssRNA). The effector protein may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel may vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

In some embodiments, methods described herein cleave a target nucleic acid at one or more locations to generate a cleaved target nucleic acid. In some embodiments, the cleaved target nucleic acid undergoes recombination (e.g., NHEJ or HDR). In some embodiments, cleavage in the target nucleic acid may be repaired (e.g., by NHEJ or HDR) without insertion of a donor nucleic acid, such that the repair results in an indel in the target nucleic acid at or near the site of the cleavage site. In some embodiments, cleavage in the target nucleic acid may be repaired (e.g., by NHEJ or HDR) with insertion of a donor nucleic acid, such that the repair results in an indel in the target nucleic acid at or near the site of the cleavage site.

In some embodiments, the mutation (e.g., indel) introduced into the target nucleic acid results in gene silencing of the target nucleic acid. Such gene silencing, in some embodiments, reduces expression of the target nucleic acid by at least 10%, at least 20%, at least 30%, at least 40%, at least 50%, at least 60%, at least 70%, at least 80%, at least 90%, or at least 95%. In some embodiments, gene silencing is accomplished by transcriptional silencing or post-transcriptional silencing. In some embodiments, the mutation (e.g., indel) introduced into the target nucleic acid occurs in both alleles of the TRAC gene, B2M gene and CIITA gene.

### CAR T Cells, Kits and Systems

The methods described herein can be used to produce an immunologically compatible CAR T or a population of such cells. Accordingly, in some aspects, provided herein is an immunologically compatible CART cell produced by a method described herein. Similarly, in some aspects, provided herein is a population of CAR T cells produced by a method described herein.

In general, CAR T cells are T cells that express a CAR. A CAR T cell can be activated in the presence of its respective antigen on a target cell, resulting in the destruction of the target cell. In some embodiments, the CAR T cell expresses CD3. In some embodiments, the CAR T cell is a naïve T cell. In some embodiments, the CAR T cell is a T-helper cells (CD4⁺ cell). In some embodiments, the CAR T cell is cytotoxic T-cells (CD8⁺ cell.) In some embodiments, the CAR T cell expresses CD4 (also referred to as a “CD4+ T cell”). In some embodiments, the CAR T cell expresses CD8 (also referred to as a “CD8+ T cell”). In some embodiments, the CAR T cell expresses CD4 and CD8 (also referred to as a “CD4+CD8+ T cell”). In some embodiments, the CAR T cell is natural killer T-cell. In some embodiments, the CAR T cell is a T-regulatory cell (T-reg).

Also provided herein, in some aspects, an immunologically compatible CAR T cell comprising: indels in each of the TRAC gene, the B2M gene, and the CIITA gene. Because of the use of the effector proteins and the guide nucleic acids described herein, in some embodiments, such a CAR T cell will include idels in each of the the TRAC gene, the B2M gene, and the CIITA gene within proximity of a PAM sequence of an effector protein described herein. Moreover, in some embodiments, such a CAR T cell will include integration of a donor nucleic acid encoding a chimeric antigen receptor (CAR) into the TRAC gene.

As described herein, effector proteins described herein can recognize specific PAM sequences. Because PAM sequences will direct the nuclease activity of the effector protein to be within or adjacent to the PAM sequences, the indels generated by the nuclease activity of the effector protein will be within proximity of a PAM sequence of an effector protein described herein. Accordingly, in some embodiments, an indel described herein will be within proximity of a PAM sequence selected from a PAM sequence comprising 5′-CTT-3′, 5′-CC-3′, 5′-TCG-3′, 5′-GCG-3′, 5′-TTG-3′, 5′-GTG-3′, 5′-ATTA-3′, 5′-ATTG-3′, 5′-GTTA-3′, 5′-GTTG-3′, 5′-TC-3′, 5′-ACTG-3′, 5′-GCTG-3′, 5′-TTC-3′, or 5′-TTT-3′. In some embodiments, an indel described herein will be within proximity of a PAM sequence comprising 5′-TBN-3′, wherein B is one or more of C, G, or T and N is any nucleotide. In some embodiments, an indel described herein will be within proximity of a PAM sequence comprising 5′-TTTN-3′, wherein N is any nucleotide. In some embodiments, an indel described herein will be within proximity of a PAM sequence comprising 5′-GTTK-3′, 5′-VTTK-3′, 5′-VTTS-3′, 5′-TTTS-3′ or 5′-VTTN-3′, wherein K is G or T, V is A, C or G, S is C or G, and N is any nucleotide.

In some embodiments, the CAR T cell provided herein comprises indels within a certain nucleotide length of the PAM sequence (either starting from the 5′ end or 3′ end of the PAM sequence, depending upon the indel location). Accordingly, in some embodiments, the indels described herein are within 10 nucleotides of the PAM sequence. In some embodiments, the indels described herein are within in some embodiments, the indels described herein are within 15 nucleotides of the PAM sequence. In some embodiments, the indels described herein are within in some embodiments, the indels described herein are within 20 nucleotides of the PAM sequence. In some embodiments, the indels described herein are within in some embodiments, the indels described herein are within 25 nucleotides of the PAM sequence. In some embodiments, the indels described herein are within in some embodiments, the indels described herein are within 30 nucleotides of the PAM sequence.

Another identifying characteristic of a CAR T cell provided herein is the location of the donor nucleic acid encoding a CAR. As described herein, use of an effector protein, guide nucleic acids and donor nucleic acid described herein, the donor nucleic acid of the CAR T cell will be in the TRAC gene. Moreover, integration of the TRAC gene can be guided by the genome editing components described here such that the sequence of the donor nucleic acid encoding the CAR is in line with the promoter of the endogenous TRAC gene. By such an integration, in some embodiments, expression of the donor nucleic acid is driven by an endogenous TRAC gene promotor of the T cell.

As described already, in some aspects, provided herein is a population of T cells comprising CAR T cells produced by a method described herein. Because of the efficiency of the methods provided herein, such a population T cells comprising the immunologically compatible CAR T cell described herein can have a high number of CAR T cells compared to the number of T cells in the population that have not been made into a CAR T cell. Accordingly, in some embodiments, at least 50% of the T cells contained in the population are an immunologically compatible CAR T cell described herein. In some embodiments, at least 55% of the T cells contained in the population are an immunologically compatible CAR T cell described herein. In some embodiments, at least 60% of the T cells contained in the population are an immunologically compatible CAR T cell described herein. In some embodiments, at least 65% of the T cells contained in the population are an immunologically compatible CAR T cell described herein. In some embodiments, at least 70% of the T cells contained in the population are an immunologically compatible CAR T cell described herein. In some embodiments, at least 75% of the T cells contained in the population are an immunologically compatible CAR T cell described herein. In some embodiments, at least 80% of the T cells contained in the population are an immunologically compatible CAR T cell described herein.

Also provided herein, in some aspects, is a kit for making an immunologically compatible chimeric antigen receptor (CAR) T cell. In some embodiments, such a kit comprises a viral vector described herein, a viral particle described herein, or a nonviral vector described herein; and one or more reagents for transducing a T cell. In some embodiments, the kit further comprises one or more containers comprising the viral vector and the one or more reagents. In some embodiments, the kit further comprises one or more containers comprising the nonviral vector and the one or more reagents. In some embodiments, the kit further comprises a package, carrier, or container that is compartmentalized to receive the one or more containers.

Also provided herein, in some aspects, is a system comprising a T cell and the viral vector described herein or the viral particle described herein. Also provided herein, in some aspects, is a system comprising a T cell and the nonviral vector described herein.

### Methods of Killing Cells and Reducing Tumor Size

Because of the antigen specificity and the immunological compatibly of the CAR T cell(s) described herein, also provided herein is a method for killing a cell or pathogen in a subject. Such a method can include administering an effective amount of an immunologically compatible CAR T cell described here or a population of immunologically compatible CAR T cells described herein to the subject. Similarly, also provided here is a method that includes: obtaining T cells from a first subject; performing a method for producing a immunologically compatible CAR T cell or population of T cells described herein; and administering an effective amount of the immunologically compatible CAR T cells back to the first subject or to a second subject.

Because of the antigen specificity, especially for cancer antigens, and the immunological compatibly of the CAR T cell(s) described herein, also provided herein a method of reducing tumor size in a subject. Such a method, in some embodiments, comprises administering an effective amount of an CAR T cell described herein or a population of CAR T cells described herein to the subject. Similarly, in some aspects, also provided herein a method of reducing tumor size in a subject that comprises: obtaining T cells from a first subject; performing a method for producing a immunologically compatible CAR T cell or population of T cells described herein; and administering an effective amount of the immunologically compatible CAR T cells back to the first subject or a second subject.

Because of the minimal number of contacting and culturing steps of the methods described herein, the time period from obtaining T cells to administration of the generated CAR T cells is shorter than other methods known in the art. For example, in some embodiments, obtaining the T cells and administering an effective amount of the immunologically compatible CAR T cells is for a period of time that is no more than 21 days. In some embodiments, obtaining the T cells and administering an effective amount of the immunologically compatible CAR T cells is for a period of time that is no more than 20 days. In some embodiments, obtaining the T cells and administering an effective amount of the immunologically compatible CAR T cells is for a period of time that is no more than 19 days. In some embodiments, obtaining the T cells and administering an effective amount of the immunologically compatible CAR T cells is for a period of time that is no more than 18 days. In some embodiments, obtaining the T cells and administering an effective amount of the immunologically compatible CAR T cells is for a period of time that is no more than 17 days. In some embodiments, obtaining the T cells and administering an effective amount of the immunologically compatible CAR T cells is for a period of time that is no more than 16 days. In some embodiments, obtaining the T cells and administering an effective amount of the immunologically compatible CAR T cells is for a period of time that is no more than 15 days. In some embodiments, obtaining the T cells and administering an effective amount of the immunologically compatible CAR T cells is for a period of time that is no more than 14 days. In some embodiments, obtaining the T cells and administering an effective amount of the immunologically compatible CAR T cells is for a period of time that is no more than 13 days. In some embodiments, obtaining the T cells and administering an effective amount of the immunologically compatible CAR T cells is for a period of time that is no more than 12 days. In some embodiments, obtaining the T cells and administering an effective amount of the immunologically compatible CAR T cells is for a period of time that is no more than 11 days. In some embodiments, obtaining the T cells and administering an effective amount of the immunologically compatible CAR T cells is for a period of time that is no more than 10 days. In some embodiments, obtaining the T cells and administering an effective amount of the immunologically compatible CAR T cells is for a period of time that is no more than 9 days. In some embodiments, obtaining the T cells and administering an effective amount of the immunologically compatible CAR T cells is for a period of time that is no more than 8 days. In some embodiments, obtaining the T cells and administering an effective amount of the immunologically compatible CAR T cells is for a period of time that is no more than 7 days. In some embodiments, obtaining the T cells and administering an effective amount of the immunologically compatible CAR T cells is for a period of time that is no more than 6 days.

In some embodiments, the T cells obtained from the subject is a naïve T cell, whereas the CAR T cell administered to the subject is a cytotoxic T cell or a helper T cell.

### Administering Cells

In some embodiments, methods comprise administering a cell or a population of cells to a subject, wherein the cell or population of cells has been contacted with or modified by a composition disclosed herein. In some embodiments, cells are administered to a subject by intravenous or parenteral injection. In some embodiments, cells are administered directly into a tumor, lymph node or site of infection.

In some embodiments, methods comprise performing leukapheresis on a subject, wherein leukocytes are collected, enriched, or depleted ex vivo to enrich T cells. The enriched T cells can be cultured to proliferate before contacting them with a composition described herein to produce autologous CAR T-cells. Cells described herein, including CAR-T cells, can be administered at a dosage of 10⁴ to 10⁹ cells/kg body weight. In some embodiments, methods comprise administering 10⁵ to 10⁶ cells/kg body weight.

Disclosed herein, in some aspects, are methods of administering a composition described herein to a subject in need thereof. Also disclosed herein, are methods of administering a cell or a population of cells comprising a composition described herein to a subject in need thereof. The subject can be a mammal. The subject can be a non-human subject. The subject can be a human subject. Methods of administering a composition or cell to a subject can be carried out in various manners, including aerosol inhalation, injection, transfusion, and implantation. The compositions and cells described herein can be administered to a subject intravenously, subcutaneously, intradermally, intratumorally, intramuscularly, or intraperitoneally. In some embodiments, compositions comprising viruses disclosed herein are administered to a subject via intravenous, parenteral, or subcutaneous injection.

In some embodiments, methods comprise administering a composition or cell described herein to a subject having cancer. The cancer can be a solid cancer (tumor). The cancer can be a blood cell cancer, including leukemias and lymphomas. Non-limiting types of cancer that could be treated with such methods and compositions include acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In some embodiments, methods comprise administering a composition or cell described herein to a subject having an infection caused by a pathogen, wherein the composition, or RNA(s) and/or protein(s) encoded by the composition, modifies a target nucleic acid of the pathogen. Non-limiting examples of pathogens are bacteria, a virus and a fungus. The target nucleic acid, in some embodiments, is a portion of a nucleic acid from a sexually transmitted infection or a contagious disease. In some embodiments, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M pneumoniae. In some embodiments, the target sequence is a portion of a gene locus of bacterium or other pathogen responsible for a disease, wherein the gene locus comprises a mutation that confers resistance to a treatment, such as antibiotic treatment.

It is understood that modifications which do not substantially affect the activity of the various embodiments described herein are also provided within the definition of the subject matter provided herein. Accordingly, the following examples are intended to illustrate but not limit the various embodiments described herein.

### Sequences and Tables

TABLE 1 provides illustrative amino acid sequences of effector proteins that are useful in the compositions, systems and methods described herein.

TABLE 1
Exemplary Amino Acid Sequence of Effector Proteins

| | SEQ | |
|---|---|---|
| | ID | |
| Name | NO | Amino Acid Sequence |
| CasM.298706 | 1 | MAKKGTNRKKMIVKVMKYELKYESGCADFNEMQNELWKLQRQTREV |
| | | MNRTIQLCYHWSYVQADYCKQHGCARRDVKPCDVYETNATSLDGYIY |
| | | QLFKDEYPNFLMANLIATLRKAHQKYDALLFDIQEGNSSIPSFKKDQPLIF |
| | | SKEAIRLPECLSDKRQITLFCFSKPYKSAHPTLDKITFAVRARSASEKSIFD |
| | | HIISGKYALGESQLVYEKKKWFFLLSYKFTPESVDVNPEKVLGVDLGVV |
| | | NALCAGSVENPHDSLFIKGTEAIEQIRRLEARKRDLQKQARYPGDGRIGH |
| | | GTKTRVSPVYQTRDAIARMQDTLNHRWSRALIDFACKKGYGTIQMEDL |
| | | SGIKALESEKPYLKHWTYFDLQSKIIYKAEEKGIRVVKVNPKCTSRRCSA |
| | | CGYISKENRKNQVEFLCVNCGYHHNADYNAAQNLSIPQIDRLIEKQLKE |
| | | QESEENEAGANPK |
| CasM.280604 | 2 | MAKGTLSKVMKYELRYLDGCGDFQNMQKELWTLQRQSREILNRTIQIA |
| | | YHWDYTDREQFKKTGQHLDIKAETGYKRLDGYIYDSLKEDVQNFASVN |
| | | VNATIQKAWAKYKSSKIDVLRGDMSLPSYKSDQPLVLHAQSMKIFSSDD |
| | | DDVLQVTLFSNAYKKACNYSNIRFIIGLHDATQRTIIKKVLSGDWGIGQS |
| | | QIVYKRPKWFLYLTYNFSPEQHEVNPDKILGVDLGESIAIYASSIGEYGSL |
| | | RIEGGEISAFAKQLEARKRSLQKQAAYCGKGRIGHGTKSRVSDVYKMED |
| | | KIANFRNTVNHRYSKMLIDYALKHMYGTIQMEDLSGIKKETGFPKFLQH |
| | | WTYYDLQQKIEAKAKEHGINFIKVDPAFTSQRCSKCGNIDSENRPSQAVF |
| | | CCKKCGYKTNADFNAS |
| CasM.281060 | 3 | MNVTKVMRYQLIYQGGGGDFESLQNQLWEFQRQTRAILNKTIQTMYLA |
| | | TANQEKFSEKALYHDLCAEYPDMISSTVNATLREATKKYRSSVREILAG |
| | | RMSLPSYKRDHPILLHNQSVALKQGNQGSYFATISVFSRKYQQGTPGVK |
| | | QPSFQLIAKDNTQRTILQRLLSGEYKLGQCQLIYIRPKWFLNVAYSFTPSE |
| | | KALDQEKVLGVDLGCVYAIYASSYGNHGIFKISGDEITSFERKQAAIQNR |
| | | AFKNDLTRIREIEERRKQKLEQARYCGEGRIGHGVKTRVAPAYQDEGKIS |
| | | RFRETINHRYSKALVDYAEKNGYGTIQMEDLSGIKSSTGFPKRLQHWTY |
| | | FDLQQKIKYKAEEQGIKVVKIKPAYTSQRCSRCGHIDPANRKSQSEFKCI |
| | | ACGFSSNADYNASQNISMRNIEKIIQGKAN |
| CasM.284933 | 4 | MAKGTITKVMKYELRYLGGFSDFHEMQKEVWQLQRQYREILNKTIQIA |
| | | LHWDYVSAQQFGESGTYLDIREETGYKTLDGYIYNCLKGAYSEMASAN |
| | | LNAAVQKAWKKYKNSKTQVLQGVMSLPSYKSDQPILIDKGNVKLSAEE |
| | | NNGRAVLTLFSRNYRDTRGLKGNVEFSVLLHDGTQKSIFRNLIDKTYAL |
| | | GQCQLVYERKKWFLLLTYSFTPAGHALDPEKILGVDLGECYALYASSCY |
| | | APGILKIEGGEIAEYALRLEKRKRSLQQQARYCGEGRIGHGTKTRVGVV |
| | | YKAEDRIASFRETINHRYSKELVDYAVSNGYGTIQMEDLSAIQKDLGFPK |
| | | RLRHWTYYDLQMKITNKAKEHGIAVVKIDPRYTSQRCSKCGHIDPANRP |
| | | RQEEFCCTACGYACNADYNASQNISIKGIEKIIQKMLSAKAD |
| CasM.287908 | 5 | MSKGMLTKVMKYTLRYVGGCGDFHEMQSILWELQKQTRAVLNKTIQIA |
| | | FEWDYRSREAFQETGEYLDVHAETGYKRLDGYIYNCLKNEYADFAGKN |
| | | LNAAIQTAWKKYNQSKRDIQTGKMSLPSYRSNQPLIIHNDNVMISQDMQ |
| | | AAPSVRFTLLSLEYKKAHDLNTNPTFEVLINDGTQRAIFEKVRSGEYKLG |
| | | QCMIQYDKKKWFLLLTYSFQPEKLTLDKNKILGVDLGETIVICASSVSER |
| | | GRFVIDGGEITRFATQIEARKRSQQHQAAYCGEGRIGHGTKTRVDAVYK |
| | | TEDRIANFRDTINHRYSRALVNYAVKHGFGTIQMEDLSGIKSSDDFPKFL |
| | | RHWTYYDLQSKIESKAKERGIAVVKVNPRFTSRRCSKCGYIDEGNRKDQ |
| | | AHFCCLSCGFRANADFNASQNLSIKGIDKIIEKEYNANSKQT |
| CasM.288518 | 6 | MGKPITKTMKYQIHYIDGCGDFHNMQKELWDLQRIVRQILNKTINESYL |
| | | WFVRSEQYYRDTGENLSVEEQTGYKTLDGHIYNLLKQEYTQKLVSNSL |
| | | NASIQAAYKKMKDSRRDVMIGTMSLPSYRSDQPIIIYNKNIKFSSHPEHGF |
| | | VVDCSLFSDAYKKSQGYEKSVKFQVSVDDNTQRSIFENILTGNYKHGQC |
| | | SIVYEKKKWFLLLTYSFVPEETKLDPDKILGVDVGVVYALYASSKGNHG |
| | | TFKIKGDEAITFIQRVEARKHSRQLQGTYCGDGRIGHGTKTRVQPVYNER |
| | | ALISNFQDTINHRYSKALIDYAKKNGYGTIQMEDLSGIKEVQQYPKYLQ |
| | | HWTYYDLQLKIQYKAKEAGIGFVKVTPKYTSQRCSHCGNIDEANRPKQ |
| | | DVFRCTVCGYERNADYNASQNLSIKGIDRIIDDQLKQMNKANPKKTENA |
| CasM.293891 | 7 | MSGGAITKVMKYDLTYKDGYGNFKDMQEAVWKLIRDTRTILNETIKIA |
| | | YHWDYLNEKSKRETGEHLDLLEETGYKRLDGYIYDDLKDRFPDFASSNL |
| | | NAAIQTAWKKYKQSQKDVYIGKMTLPSYKSDQPLPINKQSIKIYDEERE |
| | | HIVELNLFSTKHKKEHGLASNVRFRINLHDNTQHAIYERVLSGEYTLGQC |
| | | QLLYDRPKWFFILTYSFKPAQNKLDPDKILGVDMGETCALYASTFGEQG |
| | | SFVINGGEVSEYAKREEARKRSLQKQAAVCGEGRIGHGTKTRVSSVYKE |
| | | QERISNFRDTINHRYSKALIEYAVKNGCGTIQMEDLSGIRQSTDFPKFLRH |
| | | WTYYDLQQKIKTKAKETGIAVSMIDPRYTSQRCSRCGHIDKANRKDQA |
| | | HFHCLKCGYSCNADFNASQNISIRGIDKIIQKELGAKAKQTD |
| CasM.294270 | 8 | MKEIAKVMKYQLIYLDGGGDFYELQQTLWDLQRQTREILNKTIQSMYL |
| | | ATATNTAFEENALYHRFGAEYPMMAALNVNATLRTAKKRYTSTIKETL |
| | | RGTMSLPSYKRDQPILLHNQTIHLALEDGQYSALFSVYSEKFQKAHEGV |
| | | ARPRFALMARDGTQRAILDRLLDGSYRLGQSQMTYEQKKWFLSLTYKF |
| | | VPEVRELDKSKILGVDLGCVYAIYASSMQQKGIFKISGDEITEFEKRQAA |
| | | MQNREPVSTLERVEQLEQRRWQKQQQARYCGEGRVGHGTGTRVAPAY |
| | | RDADKIARFRDTINHRYSKALVEYAEKNGFGTIQMEDLSGIKEDTGFPKR |
| | | LRHWTYFDLQTKIQYKAAERGITVVKIDPQYTSQRCSRCGYIDKANRAS |
| | | QEKFLCQSCGFEANADYNASQNISVEKIDKLIAKDKKKLART |
| CasM.294491 | 9 | MGQVTKVMRYQLIYQDGGGDFYTVQQELWELQRQTREILNKTIQTMYL |
| | | ADANKEKFDNAAERTLNRRFCVDHPDMYTKTVTATLRKAKAKYNASQ |
| | | KEILAGRMSLPSYKRDQPILLNPQGFKIEEESDSFFAAIAVFSDKYKNKHP |
| | | DVDVKRLRFRLVVKDGTQRAIIRRVISGEYKLGRSQLLYSKKKWFLNVT |
| | | YSFEPAEKKVDPDKILGVDLGCVYAIYASSFGSPGVFKISGDEVSSFERK |
| | | QAAIQNRSPKSTLERVEKIEERHKQKQQQARYCGEGRIGHGTKTRIAPVY |
| | | QDEDKIARFRDTVNHRYSKALIDYAEKNGYGTIQMEDLSGIKSATGFPK |
| | | RLKHWTYYDLQTKIEYKAEERGIKVVKIDPRYTSQRCSRCGYIDSGNRK |
| | | SQAEFCCMACGFSCNADYNASQNISIGGIAKIIADKRKEADAK |
| CasM.295047 | 10 | YLDIREETGYKTLDGYIYNCLKGAYSEMASANLNAAVQKAWKKYKNS |
| | | KTQVLQGVMSLPSYKSDQPILIDKGNVKLSAEENNGRAVLTLFSRNYRD |
| | | TRGLKGNVEFSVLLHDGTQKSIFRNLIDKTYALGQCQLVYERKKWFLLL |
| | | TYSFTPAGHALDPEKILGVDLGECYALYASSCYAPGILKIEGGEIAEYALR |
| | | LEKRKRSLQQQARYCGEGRIGHGTKTRVGVVYKAEDRIASFRETINHRY |
| | | SKELVDYAVSNGYGTIQMEDLSAIQKDLGFPKRLRHWTYYDLQMKITN |
| | | KAKEHGIAVVKIDPRYTSQRCSKCGHIDPANRPRQEEFCCTACGYACNA |
| | | DYNASQNISIKGIEKIIQKMLSAKAD |
| CasM.299588 | 11 | MAEKTIVKVMKFELRYIDGAGEFSEMQKHLWELQKQTREVLNKTIQMG |
| | | YALECKRFAHHDKTGQWLDDKELTGSKYKAVADYINAELKEDYNIFYS |
| | | DCRNSTVRKAYKKFKDAKNKIFSGEMSLPSYRSNQPIIIHNRNVIIRGNAE |
| | | SALVGLKVFSDGFKALHGFPAAVNFKLCVKDGTQRAIIENVISEIYKISES |
| | | QLIYDNKKWFLILAYRFTQKKNDLNPDKILGVDLGVKFAVYASSIGEYG |
| | | SFRIKGGEVTEFIKRLEKRKKSLQNQATVCGDGRIGHGTKTRVADVYKA |
| | | RDKISNFQDTINHRYSRAIVDYARKNGYGTIQLEKLDNSIEKKGDYSPVL |
| | | VHWTYYDLRTKMEYKAAEYGIKVIAVEPKYTSQRCSKCGYISSENRKTQ |
| | | ESFECIKCGYKCNADFNASQNLSVRDIDRIIDEYLGANPELT |
| CasM.277328 | 12 | VVNVAKGALSKVMKFELSYLDGCGDFQNMQKELWTLQRQTREILNRTI |
| | | QIAYHWDYTDREHFKKTGQHLDVKSETGYKRLDGYIYDELKETVQNFA |
| | | SVNVNATIQKAWAKYKSSKTDVLRGDMSLPSYKSDQPLVLHAQSIKLSE |
| | | DKDGPVLQVTLFSNAHKKACDYSNVRFAFRLHDATQRAIFKNVLSGEY |
| | | GLGQSQIVYKRPKWFLYLTYNFSPEQHGLDPDKILGVDLGESIALYASSL |
| | | GDYGSLRIEGGEVTAFAKQLEARKRSLQKQAAHCGEGRVGHGTRARVS |
| | | DVYKAEDKIANFRNTVNHRYSKKLIEYAIQNRYGTIQMEDLSGIKQDTG |
| | | FPKFLQHWTYYDLQQKIEAKAKENGINFIKVDPSYTSQRCSKCGNIDSDN |
| | | RPSQAVFCCTKCGFRANADFNASQNLSIPEIDKIIKKERGANTK |
| CasM.297894 | 13 | MAKKGTNRKKMIVKVMKYELKYEKGCADFNEMQNELWKLQRQTREV |
| | | MNRTVQLCYHWNYVQADYCKQHGCAHRDVKPCDVYETNATSLDGYI |
| | | YQLFKDEYPNFLMANLIATLRKAHQKYDALLPDIQEGNSSIPSFKKDQPL |
| | | IFSKEAIHLPECLSDKRQITLFCFSKPYKSAHPTLDKITFAVRAHSASEKSIF |
| | | DNIINGKYALGTSQLVYEKKKWFFLLSYKFTPESVDVNPEKVLGVDLGV |
| | | VNALCAGSVENPHDSLFIKGTEAIEQIRRLEARKRDLQKQARYPGDGRIG |
| | | HGTKTRVSPVYQTRDAIARMQDTLNHRWSRALIDFACKKGYGTIQMED |
| | | LSGIKAMESEKPYLKHWTYFDLQSKIIYKAEEKGIRVVKVNPKCTSRRCS |
| | | ACGYISKENRKNQAEFLCVNCGYHHNADYNAAQNLSIPQIDRLIEKQLK |
| | | EQESEESEAGANPK |
| CasM.291449 | 14 | MTERHDNESSKIKAEVSLLNSSVPDFEKKRHVKVLKLHILKPAGDMKW |
| | | DELGALLRDARYRVFRLANLAISEAYLDFHKWRSGGNEQPKLKISQLNR |
| | | NLRSMLEDEVTGKQTKMIKSDRYSKSGALPDSIVSPLSMYKLGGLTSKS |
| | | KWSEVLRGKSSLPTFKLNMAIPVRCDKPGDRRIERTKNGDAEVELRICLQ |
| | | PYPRVIIATGRNSLGDGQRAILDRLLDNTKYSEQGYRQRCFEIKEDQRSG |
| | | KWHLFVTYDFPAIEPAKNLSRERIVGVDLGAACPLYAAINTGHARLGWK |
| | | HFSPLAARVRALQNQTIRRRRQILRGGKVSLSEDSARSGHGRKRKLKPIS |
| | | KLEGKIDRAYTTLNHQLSATVIKFAKDNGAGVVQMEDLKGLRETLTGTF |
| | | LGERWRYEELQRFIRYKADEAGIEIRLVNPQYTSRRCSECGHIHKDFTRE |
| | | FRDKSREGNKSVRFLCPDCGFTADPDYNAARNLASLDIAAIIERQLEIQG |
| | | LRKHDP |
| CasM.297599 | 15 | MKEKSKTLVKVARLRILKPAGDMKWSELGEMLRTVRYRVFRLANLAVS |
| | | EAYLGFHMYRTNRATEFKAETIGKLSRRLREMLIEEGVDEKDLSRYSQT |
| | | GAVPDTVAGALGQYKIRGITSPTKWRQVVRGQAALPTFRNDMAIPIRCD |
| | | KQYQRRLEKTEAGEIEVELMICRKPYPRIVLGTADLGPGQRAILERLLQN |
| | | TDNSADGYRQRLFEAKQDTQTKKWWLYVTYDFPRLKEGKLNQEIVVG |
| | | VDLGFSIPLYVALNIGHARLGRRHFQALGNRIRSLQRQVLARRRSIQRGG |
| | | RVNISHSTARSGHGRKRKLLPTEKLRGRIEKSYSTLNHQLSASVIDFAKN |
| | | HHAGTIQIEDLANLKEELAGTFIGARWRYHQLQQFLKYKAEEAGITLNQ |
| | | VNPRYTSRRCSECGFINIDFDRAFRDAGRTEGRVTKFLCPECGYEADPDY |
| | | NAARNISILDIDKLIRVQCKKQGLTYDAH |
| CasM.286588 | 16 | MPERPKTVNKVIWFQIHKPAGDMTWKELGNLLREARYRVFRLANLAVS |
| | | EKYLSFHMWRTGQEYKSETIGKLNRRLREMLIEEGVEEESQKRFSATGA |
| | | LPDTVVSTLAKGKLAAITSKSKWKDVVNGKTSLPTFKLNMAIPVRCDKA |
| | | EQRRLRRTESGDVELELMICKQPYPRVVLKTGKLKSGQRAILDRLVENN |
| | | DNSKEGYSQRVFEIKQVENNDGSKEWRLYISYTFPKKAVEANADVAVG |
| | | VDIGFSVPLVAAVNNGLERLGYNDFRALNERIRSLQRQVLVRRRSMQSG |
| | | GRDYVSTPTARSGHGRKRKLLPIQTLRKRWDNAYTTLNHQLSHAVVSF |
| | | AENHGAATIQIENVKSLKDELRGTFLGQRWRYFELQQFLKYKADEVGIE |
| | | LREVNARYTSRRCSECGYINMAFTRQARDKGRVDGKPMEFVCPECGYK |
| | | AHPDYNAARNIAMLDIEQKMQVQCKQQGITYADDSEVL |
| CasM.286910 | 17 | MTWPELGNMLRTVRYRVFRLANLAVSEAYLGFHMFRTKRAEEFKAET |
| | | MGKLSRRLREMLIEEGVDEKDLSRYSQTGAVPDTVAGALSQYKIRGITSP |
| | | TKWRQIVRGQVALPTFRNTMSIPVRCDKLYQRRLEQGDSGEVEVELMIC |
| | | RNPYPRVVLGTGDLNPGQQAILERLLQNTDNSADGYRQRLFEIKEDVQT |
| | | RKWWLYVTYDFPKTTGKLNPEIVVGVDLGFSIPLYVALNSGHARLGYL |
| | | HFKALGERIKSLQKQVMARRRAIQRGGRVSISHSTARTGHGVKRKLQPT |
| | | EKLRGRIEKSYSTLNHQLSASVIDFAKNHHAGVIQIEDLSGLKEQLTGTFI |
| | | GARWRYHQLQQFLKYKAEEAGITLKQINPRYTSRRCSECGFINMDFDRA |
| | | FRDAGRTYGKVTKFLCPECGYEADPDYNAARNIATLDIEKLIRVQCEKH |
| | | GLKFDAH |
| CasM.292335 | 18 | VGKEGKRNVKVMKIRILKPCDGMTWNELGQLLRDARYRVFRLANLTVS |
| | | EAYLNFHLWRTGRSQEFKKQTIGQLNRQLRNILQQEKYDDEKLNRYSKT |
| | | GALPDTVCSALWQYKLMAVMKKSKWSEVIRGKSSLPTFRNDMAIPVRC |
| | | DKPEQKRIEKTEQGQVEAALQVCVQPYPRVILGTHTLGDGQDAILKRLL |
| | | DNQNQAIGGYRQRSFEIKYDEQKRWWLFITYDFPATEVATDKTIAVGVD |
| | | LGVSVPLYAAVNNGPARLGRREFGGLGRRIRDLRNQTDARRRSIQRSGR |
| | | EGQSDDTARAGHGRKRKLLPIHILEGRLDKAYTTLNHQMSAAVIKFAAE |
| | | QGAGIIQIENLAGLQDELRGTFIGGRWRYRQLQDFLKYKTQEMGIELRQ |
| | | VNPKYTSRRCSKCGFIHKDFDRDYRNRHSENGKPAQFVCPNPDCKYESD |
| | | PDYNAARNLATLDIEEQIRVQCQKQGLEYDSKKDKNAL |
| CasM.293576 | 19 | MKEKSKTLVKVARLRILKPAGDMTWSELGEMLRTVRYRVFRLANLAVS |
| | | EAYLGFHMFRTQRAAEFKAETMGKLSRRLREMLIEEGVDEKELNCYSLT |
| | | GAVPDTVAGALHQYKIRGITSPTKWRQVVRGQAALPTFRNDMSIPIRCD |
| | | KPYQRRLEKTEAGEVEVELMICRKPYPRIVLGTADVGPGQEVILERLLQN |
| | | KDNSSDGYRQRLFEAKQDRQTGKWWLYVTYDFPRPEEGELNPEIVVGV |
| | | DLGFSVPLYVAINNGYARLGRRHFQALGNRIRSLQRQVLARRRSIQRGG |
| | | RVNISHDTARSGHGIKRKLLPTEKLRGRIEKSYSTLNHQLSASVIDFTKNH |
| | | HAGTIQIEDLANLKEVLAGTFIGARWRYHQLQQFLKYKADEAGITLKEV |
| | | NPRYTSRRCSECGFIHKDFDRAFRDSGRTDGKVARFVCPECGYGPVDPD |
| | | YNAAKNISTLDIEKHIRVQCKKQGLEYEVH |
| CasM.294537 | 20 | MKEKAKTLVKVARLRILKPAGDMTWPELGNMLRTVRYRVFRLANLAV |
| | | SEAYLGFHMFRTKRAEEFKAETMGKLSRRLREMLIEEGVDEKDLSRYSQ |
| | | TGAVPDTVAGALSQYKIRGITSPTKWRQIVRGQVALPTFRNTMSIPVRCD |
| | | KLYQRRLEQGDSGEVEVELMICRNPYPRVVLGTGDLNPGQQAILERLLQ |
| | | NTDNSADGYRQRLFEIKEDVQTRKWWLYVTYDFPKTTGKLNPEIVVGV |
| | | DLGFSIPLYVALNSGHARLGYLHFKALGERIKSLQKQVMARRRAIQRGG |
| | | RVSISHSTARTGHGVKRKLQPTEKLRGRIEKSYSTLNHQLSASVIDFAKN |
| | | HHAGVIQIEDLSGLKEQLTGTFIGARWRYHQLQQFLKYKAEEAGITLKQI |
| | | NPRYTSRRCSECGFINMDFDRAFRDAGRTYGKVTKFLCPECGYEADPDY |
| | | NAARNIATLDIEKLIRVQCEKHGLKFDAH |
| CasM.298538 | 21 | MAKKAKTMFKVTNFRILKPAGDMTWKELGQLLRDARYRTFRMANLAL |
| | | SEAYLNFYLLKKGDLKEYKNVKIGQIAKRLRDMLIEEGVDEEVQNRFSP |
| | | KVALPAYVYSALDQFKLRGLTSKSNWKKVLRGQASLPTFRLNMSVPIRC |
| | | DKPEHRRLEKTENGNVEVDLMICRKPYPRVVLETLKLDGSSKAILDRLL |
| | | ENEDNSPGNYRQRCFEVKQNPRSNDWWLYVTYEMPVDKDKKLDPKVI |
| | | VGVDLGFSVPLYVAINNGHARLGRRHFQALGKRIHNLQNQVLARRRSIQ |
| | | RGGQVNLSHSTSRSGHGRKRKLQPTEKLQQKINSAYSTLNHQLSSSVIDF |
| | | ANNHKAGTIQIEDLETLKEQLTGTYIGRQWRYYQLQQFIEYKAKENSITV |
| | | KKINPKYTSRRCSMCGHIHADFDRTFRDRSSNKGFVTKFICPECNFEADP |
| | | DYNAAKNISTLDIENKIKLQCKKQKIDY |
| CasM.19924 | 22 | MPKITRKIELLFDRSGLSEEECKEKWRFIYQINDNLYRVANRLVNQLYLA |
| | | DEIDDILRLSDQEYIALRKKLANKKLDEATRISLEEQMSQVMKRVNERRS |
| | | AILQRPQQSFAYSVVTDSDTEGLTAKILDVLKQDVLSHYKADTKEVLKG |
| | | EKSISNYKKGMPIPFAFNDSLRLYKEDGFFYLKWYNGIRFLLNFGRDASN |
| | | NQLIVERCLGISKDEISYKACSSSIQIKKKGNHSKIFLLLVVDVPVEQYAQ |
| | | KPNMVVGVDLGLNVPIYAASNSTLERKAIGSREAFLNQRGAFQRRFRAL |
| | | QRLQTTKGGRGRLHKLEPLERVREAERNWVRTQNHLFSREVINFAIDVG |
| | | ASTIQMEKLANFGRDAQGEVREDKKYVLRNWSYFELQNLIEYKAKRAG |
| | | IKVKYINPAFTSQTCSECGQLGERDSIHFKCTNPDCPNCGKDIHADYNGA |
| | | RNIAKSKDYIK |
| CasM.19952 | 23 | MPTITRKIELTLLTEGLSEEQRKEQWGLLYHINDNLYKAANNISSKLYLD |
| | | DHVSSMVRMKHAEYLSLLKELARAEKQKTPDADAIAELRKKVAAAEKE |
| | | MTDQEHAICKYATEMSTQSLSYRFATELETNIFAKILDCLKQGVFATFNS |
| | | DARDVKRGERAIRNYKKGMPIPFAWDKSLRIEKDNKDFYLRWYNGLRF |
| | | LFNFGKDRSNNRLIVERCLKMDADYDGEYKLCNSSIQIAKREGKTKLFL |
| | | LLVVKIPQEHVELNKKVVVGVDLGINVPAYVATNITEERKAIGDREHFL |
| | | NSRMAFQRRYKSLQRLRGTAGGKGRAKKLEPLERLRKAEHNWVHTQN |
| | | HLFSREVVDFAVKSHAATIHMEDLSGFGKDNDGNADERKEFVLRNWSY |
| | | YELQNMIAYKAAKYGIKVEKIHPAYTSKTCSWCGQLGFREGVTFICENP |
| | | ECKQCGEKVHADYNAARNIANSKDIIKKNE |
| CasM.274559 | 24 | MPTITRKIELTLCTDGLSEEQRKEQWGLLYHINDNLYKAANNISSKLYLD |
| | | DHVSSMVRMKHAEYLSLLKELARAEKQKTPDADAIAELKKKVAATEKE |
| | | MTDQEHAICKYATEMSTQSLSYRFSTEFETKIFAKILDCLKQGVFATFNS |
| | | DAKDVKRGERAIRNYKKGMPIPFAWTDSLRIKKDNKDFYLLWYNGLRF |
| | | LFNFGKDRSNNRLIVERCLKMDADYDGEYKLCNSSIQIAKREGKVKLFL |
| | | LLVVSIPKEHVELNKKVVVSVDLGINVPAYVATNITEERKAIGDREHFLN |
| | | SRMAFQRRYKSLQRLKGTTGGKGRTKKLEPLERLRKAEHNWVHTQNH |
| | | LFSREVVDFAVKTHAATIHMEDLSGFGKDNDGNADERKEFVLRNWSYY |
| | | ELQNMISYKAAKYGIKVEKIRPAYTSKTCSWCGQHGFREGVTFICENPA |
| | | CKQCGEKVHADYNAARNIANSKEIIKKNE |
| CasM.286251 | 25 | MPTITRKIELTLCTEGLSDQERKDQWNLLYHINDNLYRAANNISSKLYLD |
| | | DHVGSMVRLKHAEYLSLLRALEKAKKQKAPDEEVIAELSQQVATAEQE |
| | | MDEQAKAICQYATEMSTQTLSYRFATELETNIFGQILTCLRQGVFSTFNS |
| | | DARDVKRGERSIRTYKKGMPIPFPWNDSLRIGFEDGEFYLRWYNGLRFR |
| | | FDFGKDRSNNCLIVQRCMKMDKDYEGDYKLCNSSIQMVKREGKPKFFL |
| | | LLVVNIPQERVELNKNIVVGVDLGINAPAYVATNTTPERKQIGDREHFLN |
| | | ERMAFQRRFKSLQRLKGTTGGRGRAKKLEPLERLRKAEQNWVHTQNHL |
| | | FSREVIDFAVKARAATIHMEDLSGFGKDNDGNADERKEFVLRNWSYYE |
| | | LQNMITYKAAKYGIKVEKIRPAYTSKTCSWCGHQGFREGITFICENPECK |
| | | KFGEKEHADYNAARNIANSKEIIKNNEE |
| CasM.288480 | 26 | MPTITRKIELTLLTEGLSEEQRKEQWGLLYHINDNLYKAANNISSKLYLD |
| | | DHVSTMVRMKHAEYLSLLRELARAEKQKKPDVDAIAELREKVTAAEKE |
| | | MSDQERAICTYATEMSTQSLSYRFATEIETNIFAKILDCLKQGVFATFNSD |
| | | ARDVKRGERAIRNYKKGMPIPFAWDKSLRIEKDNKDFYLRWYNGLRFL |
| | | FNFGKDRSNNRLIVERCLKMDADYDGEYKLCNSSIQIVKREGKVKLFLL |
| | | LVVSIPQEHVELNKKIVVGVDLGINVPAYVATNITEERKAIGDREHFLNS |
| | | RMAFQRRYKSLQRLKGTAGGKGRTKKLEPLERLRKAEHNWVHTQNHL |
| | | FSREVVDFAVKSHAATIHMEDLSGFGKDNDGNADERKEFVLRNWSYYE |
| | | LQNMIAYKAAKYGIKVERIRPAYTSKTCSWCGQLGFREGVTFICENPEC |
| | | KQCGEKVHADYNAARNIANSKDIIKKNE |
| CasM.288668 | 27 | MPTMTRKIELKLCTEGLSDEERKAQLGLLYHINDNLYKAANNISSKLYL |
| | | DDHVSSMVRLKHAEYLSLLNEFEKAKKKGDEEQIVELSLRVAAAEKELT |
| | | DQELAICKYATEMSTDTLAYRFANEIEINVFGQILACLKQGIHSTFKKDA |
| | | ADVKRGERAIRNFKKGMPIPFPWSKSIRIENEGSDFYLRWYNGLRFRFDF |
| | | GKDRSNNRLIVSRCLNLDPDFEDEYKLSNSSLQMVKRDGRPKLFLLLVV |
| | | NIPQENVELNKKIVVGVDLGINSPAYVATNITMERQRIGSRDTFLNARMA |
| | | IQRRFQSLQKLQNTAGGRGRKKKLEPLERLKETERNWVRTQNHLFSRDV |
| | | VQFAVKTRAATIHMEDLSGFGKDDDGNADEKKEFVLRNWSYYELQTMI |
| | | KYKAAKYGIKVEKIRPAYTSRTCSWCGHEGDRKGETFICENPECEKYGK |
| | | KENADYNAARNIANSTDIIK |
| CasM.289206 | 28 | MPTITRKIELTLCTEGLSDEQRKEQWGLLYHINDNLYKAANNISSKLYLD |
| | | EHVSSMVRMKHAEYLSLLKELARAEKQQTPDEGLIAELSRKLSAAEKE |
| | | MADQELAICKYATEMSTQTLSYNFAKEIETNIFGQILTCLRQGVYATFNS |
| | | DAKDVKRGERAIRNYKKGMPIPFPWNNSLKIESDSGEFYLRWYNGLRFL |
| | | LTFGKDRSNNRMIVNRCMKMDEDFEGEYKLCNSSIQLAKRDGKPKLFLL |
| | | LVVNIPQEHVKLNKKIVVGVDLGVNVPAYVATNITEERKAIGDREHFLN |
| | | TRMAFQRRYKSLQRLKGTAGGKGRTKKLEPLERLRDAERNWVHTQNH |
| | | LFSREVVNFAVQARAATIHMEDLSGFGKDKDGNADEKKEFVLRNWSFY |
| | | ELQNMIAYKSAKYGIKVVKIRPAYTSKTCSWCGQQGDRKSTTFICENPK |
| | | CKHYGESIHADYNAARNIANSNDIVKENE |
| CasM.290598 | 29 | MPKITRKIEMTLCTEGLSDEQRKEQWGLLYHINDNLYKAANNISTKLYL |
| | | DEHVSSMVRMKHADYLSLLKELAKAEKKSPDEDLIAELREKLAAAEQE |
| | | MTDQELAICKYATEMSTQTLAYKFATEIEINVFGQILACLKQAAQSNFKS |
| | | DAKDVKRGERAIRNYKKGMPIPFPWNDNIRIDADGDEFYLRWYNGLRF |
| | | HLTFGKDKSNNRMIVKRCLKMDKDFEGEYKLCNSSIQMVKRDGKPKLF |
| | | LLLVVNIPQEHVELNKNVVVGVDLGVNVPAYVATNITEERKAIGEREHF |
| | | LNTRMQIQRRYKSLQRLKATAGGKGRTKKLEPLERLRKAEHNWVHTQN |
| | | HLFSREVVNFAVQTHAATIHMEDLSGFGKDDDGNADEQKEFVLRNWSF |
| | | YELQNMIAYKAAKYGIKVEKVKPAYTSKTCSWCGQLGFRQGVTFICENP |
| | | ACKQCGEKVHADYNAARNIANSKDIIKKNE |
| CasM.290816 | 30 | MPTITRKIELHLCTDGLTDEQQKAQRLLLYHINDNLYKAANNVSSKLYL |
| | | DEHVSSMVRLKHDEYLSLSRELARAEKKHDDELTTELRGKLAAAEREM |
| | | TDQELAICKYATEMSTQSLSYRLVTELETKIFAKILDCLKQGVYATFNSD |
| | | ARDVKRGERAIRNYKKGMPIPFAWNDSVRIEYDEKEKDFYLRWYNDIRF |
| | | KFHFGRDRSNNRLIVSRCLKLDKDYEGDYQLCNSSIQIVKRDGSTKFFLL |
| | | LVVKIPQEHVELNKRIVVGVDLGINYPAYVATNCTEERMYIGDREHFLN |
| | | TRMQFQRRYKSLQKLKGTAGGKGRSKKLEPLERLRNAERNWVHTQNH |
| | | LFSLKVVNFAVQTHAATIHLEDLSGFGKDDDGNADERKEFVLRNWSYY |
| | | ELQSMIEYKAKKYGIKVEKIRPAYTSQTCSWCGQRGFRQGVTFICENPEC |
| | | KKCGEKENADYNAARNIANSKDVIKDKNE |
| CasM.295071 | 31 | TPFVLYFQNYSLSLRQHITLYSMPTITRKIELTLCTEGLSDQERKDQWNLL |
| | | YHINDNLYRAANNISSKLYLDDHVGSMVRLKHAEYLSLLRAMEKAKKQ |
| | | KAPDEEVIAELSQQVAAAEQEMDEQAKAICQYATEMSTQTLSYRFATEL |
| | | ETNIFGQILTCLRQGVFSTFNSDARDVKRGERSIRTYKKGMPIPFPWNDSL |
| | | RIGFEDGEFYLRWYNGLRFRFDFGKDRSNNRLIVQRCMKMDKDYEGDY |
| | | KLCNSSIQMVKREGKPKFFLLLVVNIPQERVELNKNIVVGVDLGINAPAY |
| | | VATNTTPERKQIGDREHFLNERMAFQRRFKSLQRLKGTTGGRGRAKKLE |
| | | PLERLRKAEQNWVHTQNHLFSREVIDFAVKARAATIHMEDLSGFGKDR |
| | | DGNADERKEFVLRNWSYYELQNMITYKAAKYGIKVEKIRPAYTSKTCS |
| | | WCGHQGFREGITFICENPECKKFGEKEHADYNAARNIANSKEIIKNNEE |
| CasM.295231 | 32 | MPTITRKIELHLCTEELSDEQQKAQRLLLYHINDNLYKAANNVSSKLYLD |
| | | EHVSSMVRLKHDEYLSLLRELARAEKKADDELATQLREKLVAAEREMT |
| | | DQELAICKYATEMSTQSLSYRFVTELETKIFAKILDCLKQGVYATFNSDS |
| | | RDVKRGERAIRNYKKGMPIPFAWDKSVRIEYEEKEKDFFLRWYNDIRFK |
| | | FHFGRDRSNNRLIVSRCMKLDKDYEGDYQLCNSSIQIVKRDGSTKYFLLL |
| | | VVKIPQEHVELNKKIVVGVDLGINYPAFAATNCTEERMSIGDREHFLNTR |
| | | MQFQRRFKSLQRLKGTTGGKGRNKKLEPLERLRKAEHNWVHTQNHLFS |
| | | LKVVNFAVQAHAATIHLEDLSGFGKDDDGNADERKEFVLRNWSYYELQ |
| | | NMIKYKAKKFGIQVEKIRPAYTSQTCSWCGQRGFRQGITFICENPECKKC |
| | | GEKENADYNAARNIANSKDIIKDKDE |
| CasM.292139 | 33 | MPIITRKIELHISKEGLSAEDYKAQWQYLRQINDNLYMAANRVSSHCFLN |
| | | DEYKYRLCLQIPDYIDIEKQLKDSKRARLSKEELGQLKKRKKELENTVK |
| | | GRFQDEFEKNSLYTIISNEFGEIIPGQILTCLRQCVQSKYNRAKEELEKGE |
| | | RAISTYKKGMPIPFPINKSIRLQKQGEDFVLKWYNKIVFKLHFGRDRSNN |
| | | RVIVERLIQSALNDKQKGEDYVMNNSSIQLVEKDKMTKIFLLLSMDIPTQ |
| | | KRKLDSELVLGVDLGLNFPLYYATNQSANIHDHIGDKDIFLKERMVFQR |
| | | RFKELQRLQCTQGGRGRKKKLEPLEKLRDKERNWVRTKNHIFSREVIKV |
| | | ALHLGAGTIHLENLHNFGKDGNGELKNSKKFVFRNWSYFELQSMIEYK |
| | | AKMEGITVKYVNPAYTSQTCSVCGMIGERKEQAVFRCMNSSCLEYGKE |
| | | VNADFNAARNIAKAKM |
| CasM.279423 | 34 | MPTITRKIELTLCTDGLSDDLRKDQWQLLYHINDNLYKAANNISSKLYL |
| | | DEHVASMVRLKHAEYLGLIKELAKARKRADDEAVRDLCSKLAVAEQE |
| | | MNEQAKAICDYATEMSTQTLSYNFAKEIETNIFGQILTCLRQGVLLNFNS |
| | | DARDVKRGERAIRNYKKGMPIPFPWNDTIKIVSEGDEFYLRWFSGLRFH |
| | | LNFGKDRSNNRMIVRRCLKMEQDFDEEYKISNSSIQVAKRDGKQKLFLL |
| | | LVVQIPQEQVVLNKKIVVGVDLGVNVPAYVATNITEERKAIGDREHFLN |
| | | TRMQFQRRYKSLQRLKTTEGGRGRAKKLEPLERLRKAEHNWVHTQNH |
| | | LFSREVVNFALQTQAATINMEDLSGFGKDNDGNADECKEFVLRNWSYY |
| | | ELQNMIVYKASKYGIRVQKIRPAYTSKTCSWCGHMGFREGVTFICENPD |
| | | CKQFGEKVHADYNAARNIANSKEIIKNDE |
| CasM.20054 | 35 | MSKTVTKTVKIALICEHTNKYGEKVDYKDINKLLWKLQKQTRELKNKTI |
| | | QLCWEYNNFSCDYYKEHHEYPNMEDILKYKRINGFVENKLKTVNDLYS |
| | | SNCSTTILSTCNEFQNYRSEFLKGTRSINSYKSDQPLDLHKGAIKLEHDGK |
| | | DFYVSLKLLKRSAFNAMEFKGSDIRFKLNVKDKDKSTLKILESCYDKIYS |
| | | ISASKMTYDRKAGKWFLLLAYSFTPAKTENLDPEKILGVDLGIKIPICASV |
| | | YGDLDRLTIEGGKIEEFRRRVEARKRSLQKQGKQCGDGRIGHGTKKRIK |
| | | PITDIGDKIARFRDTENHIYSRYLIEYAVKKGCGTIQMEKLEGITREKDIFL |
| | | KNWTYFDLQKKIEYKAKEKGIKVVYIEPAYTSKRCSSCGFIDTDNRLDQ |
| | | AHFKCLKCGFNENADYNASQNIGIKNIDKIIKEEHKSASDKLTSE |
| CasM.282673 | 36 | VIILTKVVKLYLISEQINKEGQKIDYQRINSILWDLQKQTRDIKNRTVQLC |
| | | WEWMNFSSDYCKTQEEYPKERDILGYTLEGYVYDYFKTGYDLYTGNIS |
| | | TSSREVCSSFKNVKKEILKGERSILSYKANQPLDLHKKAISLEYDNFNFFV |
| | | KLKLLNRTGKKKYDITEDINFKIQVNDKSTRTILERCYDKEYKISGSKLIY |
| | | EKKKKLWRLNLCYSFENSQVETLEKDKILGIDLGIVYPLMASIYGEYDRF |
| | | SIKGGEIEEFRRRTEARKRSILQQTKYCGDGRIGHGRNKRTQPAYKINDKI |
| | | ARFRDTANHKYSRALIEYAVKKNCGIIQMENLTGISDNTDCFLKDWSYY |
| | | DLQTKIENKAKEMGIKVVYIKAQYTSQRCSRCGYIDVNNRIRQALFKCQ |
| | | NCGYETNADYNASQNIGMYDIENIIEETLKIQSANVKQS |
| CasM.282952 | 37 | MTKVTKVYLISEQIDKDGNKIDFKKISELLWNLQMQTRDIKNKCVQLCW |
| | | EWLNFSSDYYKKSEEYPKEKDTLGYTLSGFVYDRIKNGSDLYSSNLSTSS |
| | | RDTCTAFSNYKKEMLKGERSVLSFKANQPLDIHNKAIKLSYENGNFFVA |
| | | LKMLNRAGKEKYGIKDDLRFRMQVRDKSVRTILERLMNDEYKVSASKL |
| | | MYDKKKKLWKLNLCYSFDNHVISTLDTEKIMGVDLGVVYPIMASVNGD |
| | | YARFSIKGGEIEAFRSRVEARRRSLLNQSRYCGDGRIGHGRKKRTEPATQ |
| | | IADKIARFRDTTNHKYSRALIDYAIKNGCGTIQMEKLTGITSSAEHFLKE |
| | | WSYFDLQTKIESKAKEAGIKVVYINPKFTSQRCNKCGYIHTDNRPVQARF |
| | | CCQKCGYEENADYNASQNIGTKHIDVIIEETLKMQCEPETPTE |
| CasM.283262 | 38 | MNKVVKLALICEQSDKDNSPVDYKKINEILWELQKQTREIKNKAIQYCW |
| | | EYNNFSSDYYKKFNEYPKEKDILSYTLVGFVNDKFKTGNDLYSGNCSTT |
| | | VRNACTEFKNSKKELIKGSRSIINYRSNQPLDIHNKCIRIEFENNCFYTYLK |
| | | LLNRPAFKKYNFANTEIKFKILVRDNSTKTILERCISNEYEIAASKLLYDQ |
| | | KKKCWFLNLVYAFEIKSNNSLDPNKILGVDLGIHYPICASVYGSLDRFTI |
| | | DGGEIDEFRRRVESRKISMLKQGKNCGDGRIGHGIKARNKPVYNIEDKIA |
| | | RFRDTANHKYSRALIEYAVKHTCGTIQMEDLTGITDIANRFLKNWSYYD |
| | | LQTKIEYKAKEAGINIVYIDPKNTSRRCSKCGYIDKENRETQSRFICLKCG |
| | | FKENADYNASQNIGIKDIDKLIKEDVH |
| CasM.284833 | 39 | VTLLVKVVKIYLISEQFDKAGNQIDYKEVNKILWELQKQTREAKNKTVQ |
| | | LLWEWNNFSSDYVKASGIYPKAKDIFGYSSVHGQANKELRTKLALNSSN |
| | | LSTTTMDVCKIFNTYKKEVWEGKRSVPSYKSDQPLDLHKESIKLIYENNE |
| | | FYVRLALLKKAEFAKYGFKDGFRFKMQVKDNSTKTILERCFDEVYKINA |
| | | SKLLYDQKKKKWKLNLSYSFDNKNISELDKEKILGVDVGVNCPLVASVF |
| | | GDRDRFIIKGGEIEKFRKSVEARRRSMLEQTKYCGDGRIGHGRKKRTEPA |
| | | LNIGDKIARFRDTTNHKYSRALIEYAVKKGCGTIQMEKLTGITSKSDRFL |
| | | KDWTYYDLQTKIENKAKEVGINVVYIAPKYTSQRCSKCGYIHKDNRPNQ |
| | | AKFRCLECDFESNADYNASQNIGIKNIDKIIEKDLQKQESEVQVNENK |
| CasM.287700 | 40 | MNKVVKLALICEQSDKNNSPVDYKKVNEILWELQKQTREIKNKTIQYC |
| | | WEYYNFSSDYYKKFNKYPKEKDILSYTLWGFINDKFKTGNDLYSGNCS |
| | | ATTKKVIKEFKNSKKELIRGSRSIINYKSNQPLNIHNKCIHLQFKNNNFYV |
| | | SINLLNRRSFKKYNFANTAIKFKILVRDNSTKAILERCISNEYKISESQLIY |
| | | NKKKKCWFLNLSYAFEIKSNNSLDPNKILGVDLGIHYPICASVYGSLDRF |
| | | TIDGGEIDEFRRRVESRKISMLKQGKNCGDGRIGHGIKARNKPVYNIEDK |
| | | IARFRDTANHKYSRALIEYAVKNNCGTIQMEDLTGITDNANRFLKNWSY |
| | | YDLQTKIEYKAKEASINVVYINPENTSRRCSKCGYIDKENRKTQSSFICLK |
| | | CGFKENADYNASQNISIKDIDKLIKEDVH |
| CasM.291507 | 41 | VTLLVKVVKIHLISEQFDKAGNRIDYEEVNKILWELQKQTREAKNKTVQ |
| | | LLWEWNNFSSDYVKASGIYPKAKDIFGYSSVHGQANKELRTKLALNSSN |
| | | LSTTTMDVCKNFNTYKKEVWKGKRSVPSYKSDQPLDLHKDSIKLIYENN |
| | | QFYVRLALLKKAEFAKYGFKDGFHFKMQVKDNSTKTILERCFDEVYKIN |
| | | ASKLLYDQKKKKWKLNLSYSFDNKNISELDKEKILGVDVGVSYPLVASV |
| | | FGDRDRFKIKGGEIEKFRKSVEARRRSMLEQTKYCGDGRIGHGRKKRTE |
| | | PALNIGDKIARFRDTTNHKYSRALIEYAVKKGCGTIQMEKLTGITSKADR |
| | | FLKDWTYYDLQTKIENKAKEVGINVVYIAPKYTSQRCSKCGYIHKDNRP |
| | | NQAKFRCLECDFESNADYNASQNIGIKNIDKIIEKDLQKQESEVQVNENK |
| CasM.293410 | 42 | LIWKDALGGIILTKIVKLYLISEQIDKDGNRVDYKEINSILWNLQKQTRDI |
| | | KNKTVQLCWEWMNFSSDYYKKNELYPNEKEILNLTLRGYAYDHFKQG |
| | | YDLYSSNISVLTEAVCGAFKNAKKEMLNGEKSVLSYKAEQPLDIHKKCI |
| | | KLEYDKNFYVKLKMLNKAGKKKYGIEDDLNFKIQVEDKSTRTILERCID |
| | | GEYVVSGSKLIYDKKKKLWKLNLCYSFKANEIESLDKNKILGIDLGIACP |
| | | LMASVNGEFDRFSIKGGEIETFRKRIEARKRSVLHQTKYCGDGRIGHGRN |
| | | KRTEPAYKINDKIARFRDTANHKYSRALIDYAIRKNCGMIQMENLTGISD |
| | | KKEHFLKEWSYYDLQTKIENKAKEKGIKIVYINPEYTSQRCSKCGYIDAN |
| | | NRELRAVFKCQKCGFEADADYNASQNIGIKNIEDIIENTLKISSANEKQTK |
| | | NT |
| CasM.295105 | 43 | VFYSTFLCYILTKYIDFSANECYNINTSSEVKQLMNKVVKLALICEQSDK |
| | | DNSPVDYKKINEILWELQKQTREIKNKAIQYCWEYNNFSSDYYKKFNEY |
| | | PKEKDILSYTLVGFVNDKFKTGNDLYSGNCSTTVRNACTEFKNSKKELIK |
| | | GSRSIINYRSNQPLDIHNKCIRIEFENNCFYTYLKLLNRPAFKKYNFANTEI |
| | | KFKILVRDNSTKTILERCISNEYEIAASKLLYDQKKKCWFLNLVYAFEIKS |
| | | NNSLDPNKILGVDLGIHYPICASVYGSLDRFTIDGGEIDEFRRRVESRKIS |
| | | MLKQGKNCGDGRIGHGIKARNKPVYNIEDKIARFRDTANHKYSRALIEY |
| | | AVKHTCGTIQMEDLTGITDIANRFLKNWSYYDLQTKIEYKAKEAGINIVY |
| | | IDPKNTSRRCSKCGYIDKENRETQSRFICLKCGFKENADYNASQNIGIKDI |
| | | DKLIKEDVH |
| CasM.295187 | 44 | LISEQIDKDGNRVDYKEINSILWNLQKQTRDIKNKTVQLCWEWMNFSSD |
| | | YYKKNELYPNEKEILNLTLRGYAYDHFKQGYDLYSSNISVLTEAVCGAF |
| | | KNAKKEMLNGEKSVLSYKAEQPLDIHKKCIKLEYDKNFYVKLKMLNKA |
| | | GKKKYGIEDDLNFKIQVEDKSTRTILERCIDGEYVVSGSKLIYDKKKKLW |
| | | KLNLCYSFKANEIESLDKNKILGIDLGIACPLMASVNGEFDRFSIKGGEIE |
| | | TFRKRIEARKRSVLHQTKYCGDGRIGHGRNKRTEPAYKINDKIARFRDT |
| | | ANHKYSRALIDYAIRKNCGMIQMENLTGISDNKEHFLKEWSYYDLQTKI |
| | | ENKAKEKGIKIVYINPEYTSQRCSKCGYIDANNRELRAVFKCQNCGFEA |
| | | DADYNASQNIGIKNIEDIIENTLKISSANEKQTKNT |
| CasM.295929 | 45 | LVKVVKIYLISEQVDEQGKDVDYNTICGVLWDLQWETREIKNKTVQLC |
| | | WEWSGFSSDYYKKYGEYPKEKNLLDYTMGGFVYDKLKSKYHLYTANL |
| | | STTSQNTCGIFRTYKVDFVKGNRSVLSFKADQPLDVHKKSISIDRIDDNY |
| | | FVKLKLLNKSGIQKYGIRDDFHFRMLVKDNSTKTILERCVGGDYKAAAS |
| | | KIIYDKKKKMWCLNLSYEFDVNTAKDLNKNRILGIDIGIVYPVVASVNG |
| | | ELDRFVIQGGEIETFRRRVENRKKSLLKQTKYCGDGRIGHGRNKRTEPV |
| | | DIISDQIARFRNTANHKYSRAVIDYAVRKQCGTIQMENLKGITDKSDRFL |
| | | KNWSYYDLQQKIEYKAKEKGINVVFINPKYTSQRCSRCGYIDSANRPKL |
| | | PNQSKFLCIKCGFTENADYNASQNIALYNIEKLIDAEA |
| CasΦ.1 | 46 | MADTPTLFTQFLRHHLPGQRFRKDILKQAGRILANKGEDATIAFLRGKSE |
| | | ESPPDFQPPVKCPIIACSRPLTEWPIYQASVAIQGYVYGQSLAEFEASDPG |
| | | CSKDGLLGWFDKTGVCTDYFSVQGLNLIFQNARKRYIGVQTKVTNRNE |
| | | KRHKKLKRINAKRIAEGLPELTSDEPESALDETGHLIDPPGLNTNIYCYQQ |
| | | VSPKPLALSEVNQLPTAYAGYSTSGDDPIQPMVTKDRLSISKGQPGYIPE |
| | | HQRALLSQKKHRRMRGYGLKARALLVIVRIQDDWAVIDLRSLLRNAYW |
| | | RRIVQTKEPSTITKLLKLVTGDPVLDATRMVATFTYKPGIVQVRSAKCLK |
| | | NKQGSKLFSERYLNETVSVTSIDLGSNNLVAVATYRLVNGNTPELLQRF |
| | | TLPSHLVKDFERYKQAHDTLEDSIQKTAVASLPQGQQTEIRMWSMYGFR |
| | | EAQERVCQELGLADGSIPWNVMTATSTILTDLFLARGGDPKKCMFTSEP |
| | | KKKKNSKQVLYKIRDRAWAKMYRTLLSKETREAWNKALWGLKRGSPD |
| | | YARLSKRKEELARRCVNYTISTAEKRAQCGRTIVALEDLNIGFFHGRGK |
| | | QEPGWVGLFTRKKENRWLMQALHKAFLELAHHRGYHVIEVNPAYTSQ |
| | | TCPVCRHCDPDNRDQHNREAFHCIGCGFRGNADLDVATHNIAMVAITG |
| | | ESLKRARGSVASKTPQPLAAE |
| CasΦ.2 | 47 | MPKPAVESEFSKVLKKHFPGERFRSSYMKRGGKILAAQGEEAVVAYLQ |
| | | GKSEEEPPNFQPPAKCHVVTKSRDFAEWPIMKASEAIQRYIYALSTTERA |
| | | ACKPGKSSESHAAWFAATGVSNHGYSHVQGLNLIFDHTLGRYDGVLKK |
| | | VQLRNEKARARLESINASRADEGLPEIKAEEEEVATNETGHLLQPPGINPS |
| | | FYVYQTISPQAYRPRDEIVLPPEYAGYVRDPNAPIPLGVVRNRCDIQKGC |
| | | PGYIPEWQREAGTAISPKTGKAVTVPGLSPKKNKRMRRYWRSEKEKAQ |
| | | DALLVTVRIGTDWVVIDVRGLLRNARWRTIAPKDISLNALLDLFTGDPVI |
| | | DVRRNIVTFTYTLDACGTYARKWTLKGKQTKATLDKLTATQTVALVAI |
| | | DLGQTNPISAGISRVTQENGALQCEPLDRFTLPDDLLKDISAYRIAWDRN |
| | | EEELRARSVEALPEAQQAEVRALDGVSKETARTQLCADFGLDPKRLPW |
| | | DKMSSNTTFISEALLSNSVSRDQVFFTPAPKKGAKKKAPVEVMRKDRTW |
| | | ARAYKPRLSVEAQKLKNEALWALKRTSPEYLKLSRRKEELCRRSINYVI |
| | | EKTRRRTQCQIVIPVIEDLNVRFFHGSGKRLPGWDNFFTAKKENRWFIQG |
| | | LHKAFSDLRTHRSFYVFEVRPERTSITCPKCGHCEVGNRDGEAFQCLSCG |
| | | KTCNADLDVATHNLTQVALTGKTMPKREEPRDAQGTAPARKTKKASKS |
| | | KAPPAEREDQTPAQEPSQTS |
| CasΦ.3 | 48 | MYILEMADLKSEPSLLAKLLRDRFPGKYWLPKYWKLAEKKRLTGGEEA |
| | | ACEYMADKQLDSPPPNFRPPARCVILAKSRPFEDWPVHRVASKAQSFVI |
| | | GLSEQGFAALRAAPPSTADARRDWLRSHGASEDDLMALEAQLLETIMG |
| | | NAISLHGGVLKKIDNANVKAAKRLSGRNEARLNKGLQELPPEQEGSAYG |
| | | ADGLLVNPPGLNLNIYCRKSCCPKPVKNTARFVGHYPGYLRDSDSILISG |
| | | TMDRLTIIEGMPGHIPAWQREQGLVKPGGRRRRLSGSESNMRQKVDPST |
| | | GPRRSTRSGTVNRSNQRTGRNGDPLLVEIRMKEDWVLLDARGLLRNLR |
| | | WRESKRGLSCDHEDLSLSGLLALFSGDPVIDPVRNEVVFLYGEGIIPVRST |
| | | KPVGTRQSKKLLERQASMGPLTLISCDLGQTNLIAGRASAISLTHGSLGV |
| | | RSSVRIELDPEIIKSFERLRKDADRLETEILTAAKETLSDEQRGEVNSHEK |
| | | DSPQTAKASLCRELGLHPPSLPWGQMGPSTTFIADMLISHGRDDDAFLSH |
| | | GEFPTLEKRKKFDKRFCLESRPLLSSETRKALNESLWEVKRTSSEYARLS |
| | | QRKKEMARRAVNFVVEISRRKTGLSNVIVNIEDLNVRIFHGGGKQAPGW |
| | | DGFFRPKSENRWFIQAIHKAFSDLAAHHGIPVIESDPQRTSMTCPECGHC |
| | | DSKNRNGVRFLCKGCGASMDADFDAACRNLERVALTGKPMPKPSTSCE |
| | | RLLSATTGKVCSDHSLSHDAIEKAS |
| CasΦ.4 | 49 | MEKEITELTKIRREFPNKKFSSTDMKKAGKLLKAEGPDAVRDFLNSCQEI |
| | | IGDFKPPVKTNIVSISRPFEEWPVSMVGRAIQEYYFSLTKEELESVHPGTS |
| | | SEDHKSFFNITGLSNYNYTSVQGLNLIFKNAKAIYDGTLVKANNKNKKL |
| | | EKKFNEINHKRSLEGLPIITPDFEEPFDENGHLNNPPGINRNIYGYQGCAA |
| | | KVFVPSKHKMVSLPKEYEGYNRDPNLSLAGFRNRLEIPEGEPGHVPWFQ |
| | | RMDIPEGQIGHVNKIQRFNFVHGKNSGKVKFSDKTGRVKRYHHSKYKD |
| | | ATKPYKFLEESKKVSALDSILAHITIGDDWVVFDIRGLYRNVFYRELAQK |
| | | GLTAVQLLDLFTGDPVIDPKKGVVTFSYKEGVVPVFSQKIVPRFKSRDTL |
| | | EKLTSQGPVALLSVDLGQNEPVAARVCSLKNINDKITLDNSCRISFLDDY |
| | | KKQIKDYRDSLDELEIKIRLEAINSLETNQQVEIRDLDVFSADRAKANTV |
| | | DMFDIDPNLISWDSMSDARVSTQISDLYLKNGGDESRVYFEINNKRIKRS |
| | | DYNISQLVRPKLSDSTRKNLNDSIWKLKRTSEEYLKLSKRKLELSRAVVN |
| | | YTIRQSKLLSGINDIVIILEDLDVKKKFNGRGIRDIGWDNFFSSRKENRWFI |
| | | PAFHKAFSELSSNRGLCVIEVNPAWTSATCPDCGFCSKENRDGINFTCRK |
| | | CGVSYHADIDVATLNIARVAVLGKPMSGPADRERLGDTKKPRVARSRK |
| | | TMKRKDISNSTVEAMVTA |
| CasΦ.5 | 50 | MDMLDTETNYATETPAQQQDYSPKPPKKAQRAPKGFSKKARPEKKPPK |
| | | PITLFTQKHFSGVRFLKRVIRDASKILKLSESRTITFLEQAIERDGSAPPDV |
| | | TPPVHNTIMAVTRPFEEWPEVILSKALQKHCYALTKKIKIKTWPKKGPGK |
| | | KCLAAWSARTKIPLIPGQVQATNGLFDRIGSIYDGVEKKVTNRNANKKL |
| | | EYDEAIKEGRNPAVPEYETAYNIDGTLINKPGYNPNLYITQSRTPRLITEA |
| | | DRPLVEKILWQMVEKKTQSRNQARRARLEKAAHLQGLPVPKFVPEKVD |
| | | RSQKIEIRIIDPLDKIEPYMPQDRMAIKASQDGHVPYWQRPFLSKRRNRR |
| | | VRAGWGKQVSSIQAWLTGALLVIVRLGNEAFLADIRGALRNAQWRKLL |
| | | KPDATYQSLFNLFTGDPVVNTRINHLTMAYREGVVNIVKSRSFKGRQTR |
| | | EHLLTLLGQGKTVAGVSFDLGQKHAAGLLAAHFGLGEDGNPVFTPIQAC |
| | | FLPQRYLDSLTNYRNRYDALTLDMRRQSLLALTPAQQQEFADAQRDPG |
| | | GQAKRACCLKLNLNPDEIRWDLVSGISTMISDLYIERGGDPRDVHQQVE |
| | | TKPKGKRKSEIRILKIRDGKWAYDFRPKIADETRKAQREQLWKLQKASS |
| | | EFERLSRYKINIARAIANWALQWGRELSGCDIVIPVLEDLNVGSKFFDGK |
| | | GKWLLGWDNRFTPKKENRWFIKVLHKAVAELAPHRGVPVYEVMPHRT |
| | | SMTCPACHYCHPTNREGDRFECQSCHVVKNTDRDVAPYNILRVAVEGK |
| | | TLDRWQAEKKPQAEPDRPMILIDNQES |
| CasΦ.6 | 51 | MDMLDTETNYATETPAQQQDYSPKPPKKAQRAPKGFSKKARPEKKPPK |
| | | PITLFTQKHFSGVRFLKRVIRDASKILKLSESRTITFLEQAIERDGSAPPDV |
| | | TPPVHNTIMAVTRPFEEWPEVILSKALQKHCYALTKKIKIKTWPKKGPGK |
| | | KCLAAWSARTKIPLIPGQVQATNGLFDRIGSIYDGVEKKVTNRNANKKL |
| | | EYDEAIKEGRNPAVPEYETAYNIDGTLINKPGYNPNLYITQSRTPRLITEA |
| | | DRPLVEKILWQMVEKKTQSRNQARRARLEKAAHLQGLPVPKFVPEKVD |
| | | RSQKIEIRIIDPLDKIEPYMPQDRMAIKASQDGHVPYWQRPFLSKRRNRR |
| | | VRAGWGKQVSSIQAWLTGALLVIVRLGNEAFLADIRGALRNAQWRKLL |
| | | KPDATYQSLFNLFTGDPVVNTRTNHLTMAYREGVVDIVKSRSFKGRQTR |
| | | EHLLTLLGQGKTVAGVSFDLGQKHAAGLLAAHFGLGEDGNPVFTPIQAC |
| | | FLPQRYLDSLTNYRNRYDALTLDMRRQSLLALTPAQQQEFADAQRDPG |
| | | GQAKRACCLKLNLNPDEIRWDLVSGISTMISDLYIERGGDPRDVHQQVE |
| | | TKPKGKRKSEIRILKIRDGKWAYDFRPKIADETRKAQREQLWKLQKASS |
| | | EFERLSRYKINIARAIANWALQWGRELSGCDIVIPVLEDLNVGSKFFDGK |
| | | GKWLLGWDNRFTPKKENRWFIKVLHKAVAELAPHKGVPVYEVMPHRT |
| | | SMTCPACHYCHPTNREGDRFECQSCHVVKNTDRDVAPYNILRVAVEGK |
| | | TLDRWQAEKKPQAEPDRPMILIDNQES |
| CasΦ.7 | 52 | MSSLPTPLELLKQKHADLFKGLQFSSKDNKMAGKVLKKDGEEAALAFL |
| | | SERGVSRGELPNFRPPAKTLVVAQSRPFEEFPIYRVSEAIQLYVYSLSVKE |
| | | LETVPSGSSTKKEHQRFFQDSSVPDFGYTSVQGLNKIFGLARGIYLGVITR |
| | | GENQLQKAKSKHEALNKKRRASGEAETEFDPTPYEYMTPERKLAKPPG |
| | | VNHSIMCYVDISVDEFDFRNPDGIVLPSEYAGYCREINTAIEKGTVDRLG |
| | | HLKGGPGYIPGHQRKESTTEGPKINFRKGRIRRSYTALYAKRDSRRVRQG |
| | | KLALPSYRHHMMRLNSNAESAILAVIFFGKDWVVFDLRGLLRNVRWRN |
| | | LFVDGSTPSTLLGMFGDPVIDPKRGVVAFCYKEQIVPVVSKSITKMVKAP |
| | | ELLNKLYLKSEDPLVLVAIDLGQTNPVGVGVYRVMNASLDYEVVTRFA |
| | | LESELLREIESYRQRTNAFEAQIRAETFDAMTSEEQEEITRVRAFSASKAK |
| | | ENVCHRFGMPVDAVDWATMGSNTIHIAKWVMRHGDPSLVEVLEYRKD |
| | | NEIKLDKNGVPKKVKLTDKRIANLTSIRLRFSQETSKHYNDTMWELRRK |
| | | HPVYQKLSKSKADFSRRVVNSIIRRVNHLVPRARIVFIIEDLKNLGKVFHG |
| | | SGKRELGWDSYFEPKSENRWFIQVLHKAFSETGKHKGYYIIECWPNWTS |
| | | CTCPKCSCCDSENRHGEVFRCLACGYTCNTDFGTAPDNLVKIATTGKGL |
| | | PGPKKRCKGSSKGKNPKIARSSETGVSVTESGAPKVKKSSPTQTSQSSSQ |
| | | SAP |
| CasΦ.8 | 53 | MNKIEKEKTPLAKLMNENFAGLRFPFAIIKQAGKKLLKEGELKTIEYMTG |
| | | KGSIEPLPNFKPPVKCLIVAKRRDLKYFPICKASCEIQSYVYSLNYKDFMD |
| | | YFSTPMTSQKQHEEFFKKSGLNIEYQNVAGLNLIFNNVKNTYNGVILKV |
| | | KNRNEKLKKKAIKNNYEFEEIKTFNDDGCLINKPGINNVIYCFQSISPKIL |
| | | KNITHLPKEYNDYDCSVDRNIIQKYVSRLDIPESQPGHVPEWQRKLPEFN |
| | | NTNNPRRRRKWYSNGRNISKGYSVDQVNQAKIEDSLLAQIKIGEDWIILD |
| | | IRGLLRDLNRRELISYKNKLTIKDVLGFFSDYPIIDIKKNLVTFCYKEGVIQ |
| | | VVSQKSIGNKKSKQLLEKLIENKPIALVSIDLGQTNPVSVKISKLNKINNKI |
| | | SIESFTYRFLNEEILKEIEKYRKDYDKLELKLINEA |
| CasΦ.9 | 54 | MDMLDTETNYATETPSQQQDYSPKPPKKDRRAPKGFSKKARPEKKPPKP |
| | | ITLFTQKHFSGVRFLKRVIRDASKILKLSESRTITFLEQAIERDGSAPPDVT |
| | | PPVHNTIMAVTRPFEEWPEVILSKALQKHCYALTKKIKIKTWPKKGPGK |
| | | KCLAAWSARTKIPLIPGQVQATNGLFDRIGSIYDGVEKKVTNRNANKKL |
| | | EYDEAIKEGRNPAVPEYETAYNIDGTLINKPGYNPNLYITQSRTPRLITEA |
| | | DRPLVEKILWQMVEKKTQSRNQARRARLEKAAHLQGLPVPKFVPEKVD |
| | | RSQKIEIRIIDPLDKIEPYMPQDRMAIKASQDGHVPYWQRPFLSKRRNRR |
| | | VRAGWGKQVSSIQAWLTGALLVIVRLGNEAFLADIRGALRNAQWRKLL |
| | | KPDATYQSLFNLFTGDPVVNTRTNHLTMAYREGVVDIVKSRSFKGRQTR |
| | | EHLLTLLGQGKTVAGVSFDLGQKHAAGLLAAHFGLGEDGNPVFTPIQAC |
| | | FLPQRYLDSLTNYRNRYDALTLDMRRQSLLALTPAQQQEFADAQRDPG |
| | | GQAKRACCLKLNLNPDEIRWDLVSGISTMISDLYIERGGDPRDVHQQVE |
| | | TKPKGKRKSEIRILKIRDGKWAYDFRPKIADETRKAQREQLWKLQKASS |
| | | EFERLSRYKINIARAIANWALQWGRELSGCDIVIPVLEDLNVGSKFFDGK |
| | | GKWLLGWDNRFTPKKENRWFIKVLHKAVAELAPHRGVPVYEVMPHRT |
| | | SMTCPACHYCHPTNREGDRFECQSCHVVKNTDRDVAPYNILRVAVEGK |
| | | TLDRWQAEKKPQAEPDRPMILIDNQES |
| CasΦ.10 | 55 | MDMLDTETNYATETPSQQQDYSPKPPKKDRRAPKGFSKKARPEKKPPKP |
| | | ITLFTQKHFSGVRFLKRVIRDASKILKLSESRTITFLEQAIERDGSAPPDVT |
| | | PPVHNTIMAVTRPFEEWPEVILSKALQKHCYALTKKIKIKTWPKKGPGK |
| | | KCLAAWSARTKIPLIPGQVQATNGLFDRIGSIYDGVEKKVTNRNANKKL |
| | | EYDEAIKEGRNPAVPEYETAYNIDGTLINKPGYNPNLYITQSRTPRLITEA |
| | | DRPLVEKILWQMVEKKTQSRNQARRARLEKAAHLQGLPVPKFVPEKVD |
| | | RSQKIEIRIIDPLDKIEPYMPQDRMAIKASQDGHVPYWQRPFLSKRRNRR |
| | | VRAGWGKQVSSIQAWLTGALLVIVRLGNEAFLADIRGALRNAQWRKLL |
| | | KPDATYQSLFNLFTGDPVVNTRTNHLTMAYREGVVNIVKSRSFKGRQTR |
| | | EHLLTLLGQGKTVAGVSFDLGQKHAAGLLAAHFGLGEDGNPVFTPIQAC |
| | | FLPQRYLDSLTNYRNRYDALTLDMRRQSLLALTPAQQQEFADAQRDPG |
| | | GQAKRACCLKLNLNPDEIRWDLVSGISTMISDLYIERGGDPRDVHQQVE |
| | | TKPKGKRKSEIRILKIRDGKWAYDFRPKIADETRKAQREQLWKLQKASS |
| | | EFERLSRYKINIARAIANWALQWGRELSGCDIVIPVLEDLNVGSKFFDGK |
| | | GKWLLGWDNRFTPKKENRWFIKVLHKAVAELAPHRGVPVYEVMPHRT |
| | | SMTCPACHYCHPTNREGDRFECQSCHVVKNTDRDVAPYNILRVAVEGK |
| | | TLDRWQAEKKPQAEPDRPMILIDNQES |
| CasΦ.11 | 56 | MSNKTTPPSPLSLLLRAHFPGLKFESQDYKIAGKKLRDGGPEAVISYLTG |
| | | KGQAKLKDVKPPAKAFVIAQSRPFIEWDLVRVSRQIQEKIFGIPATKGRP |
| | | KQDGLSETAFNEAVASLEVDGKSKLNEETRAAFYEVLGLDAPSLHAQA |
| | | QNALIKSAISIREGVLKKVENRNEKNLSKTKRRKEAGEEATFVEEKAHDE |
| | | RGYLIHPPGVNQTIPGYQAVVIKSCPSDFIGLPSGCLAKESAEALTDYLPH |
| | | DRMTIPKGQPGYVPEWQHPLLNRRKNRRRRDWYSASLNKPKATCSKRS |
| | | GTPNRKNSRTDQIQSGRFKGAIPVLMRFQDEWVIIDIRGLLRNARYRKLL |
| | | KEKSTIPDLLSLFTGDPSIDMRQGVCTFIYKAGQACSAKMVKTKNAPEIL |
| | | SELTKSGPVVLVSIDLGQTNPIAAKVSRVTQLSDGQLSHETLLRELLSNDS |
| | | SDGKEIARYRVASDRLRDKLANLAVERLSPEHKSEILRAKNDTPALCKA |
| | | RVCAALGLNPEMIAWDKMTPYTEFLATAYLEKGGDRKVATLKPKNRPE |
| | | MLRRDIKFKGTEGVRIEVSPEAAEAYREAQWDLQRTSPEYLRLSTWKQE |
| | | LTKRILNQLRHKAAKSSQCEVVVMAFEDLNIKMMHGNGKWADGGWD |
| | | AFFIKKRENRWFMQAFHKSLTELGAHKGVPTIEVTPHRTSITCTKCGHCD |
| | | KANRDGERFACQKCGFVAHADLEIATDNIERVALTGKPMPKPESERSGD |
| | | AKKSVGARKAAFKPEEDAEAAE |
| CasΦ.12 | 57 | MIKPTVSQFLTPGFKLIRNHSRTAGLKLKNEGEEACKKFVRENEIPKDEC |
| | | PNFQGGPAIANIIAKSREFTEWEIYQSSLAIQEVIFTLPKDKLPEPILKEEW |
| | | RAQWLSEHGLDTVPYKEAAGLNLIIKNAVNTYKGVQVKVDNKNKNNL |
| | | AKINRKNEIAKLNGEQEISFEEIKAFDDKGYLLQKPSPNKSIYCYQSVSPK |
| | | PFITSKYHNVNLPEEYIGYYRKSNEPIVSPYQFDRLRIPIGEPGYVPKWQY |
| | | TFLSKKENKRRKLSKRIKNVSPILGIICIKKDWCVFDMRGLLRTNHWKKY |
| | | HKPTDSINDLFDYFTGDPVIDTKANVVRFRYKMENGIVNYKPVREKKGK |
| | | ELLENICDQNGSCKLATVDVGQNNPVAIGLFELKKVNGELTKTLISRHPT |
| | | PIDFCNKITAYRERYDKLESSIKLDAIKQLTSEQKIEVDNYNNNFTPQNTK |
| | | QIVCSKLNINPNDLPWDKMISGTHFISEKAQVSNKSEIYFTSTDKGKTKD |
| | | VMKSDYKWFQDYKPKLSKEVRDALSDIEWRLRRESLEFNKLSKSREQD |
| | | ARQLANWISSMCDVIGIENLVKKNNFFGGSGKREPGWDNFYKPKKENR |
| | | WWINAIHKALTELSQNKGKRVILLPAMRTSITCPKCKYCDSKNRNGEKF |
| | | NCLKCGIELNADIDVATENLATVAITAQSMPKPTCERSGDAKKPVRARK |
| | | AKAPEFHDKLAPSYTVVLREAV |
| CasΦ.13 | 58 | MRQPAEKTAFQVFRQEVIGTQKLSGGDAKTAGRLYKQGKMEAAREWL |
| | | LKGARDDVPPNFQPPAKCLVVAVSHPFEEWDISKTNHDVQAYIYAQPLQ |
| | | AEGHLNGLSEKWEDTSADQHKLWFEKTGVPDRGLPVQAINKIAKAAVN |
| | | RAFGVVRKVENRNEKRRSRDNRIAEHNRENGLTEVVREAPEVATNADG |
| | | FLLHPPGIDPSILSYASVSPVPYNSSKHSFVRLPEEYQAYNVEPDAPIPQFV |
| | | VEDRFAIPPGQPGYVPEWQRLKCSTNKHRRMRQWSNQDYKPKAGRRA |
| | | KPLEFQAHLTRERAKGALLVVMRIKEDWVVFDVRGLLRNVEWRKVLSE |
| | | EAREKLTLKGLLDLFTGDPVIDTKRGIVTFLYKAEITKILSKRTVKTKNAR |
| | | DLLLRLTEPGEDGLRREVGLVAVDLGQTHPIAAAIYRIGRTSAGALESTV |
| | | LHRQGLREDQKEKLKEYRKRHTALDSRLRKEAFETLSVEQQKEIVTVSG |
| | | SGAQITKDKVCNYLGVDPSTLPWEKMGSYTHFISDDFLRRGGDPNIVHF |
| | | DRQPKKGKVSKKSQRIKRSDSQWVGRMRPRLSQETAKARMEADWAAQ |
| | | NENEEYKRLARSKQELARWCVNTLLQNTRCITQCDEIVVVIEDLNVKSL |
| | | HGKGAREPGWDNFFTPKTENRWFIQILHKTFSELPKHRGEHVIEGCPLRT |
| | | SITCPACSYCDKNSRNGEKFVCVACGATFHADFEVATYNLVRLATTGMP |
| | | MPKSLERQGGGEKAGGARKARKKAKQVEKIVVQANANVTMNGASLHS |
| | | P |
| CasΦ.14 | 59 | MSSLPTPLELLKQKHADLFKGLQFSSKDNKMAGKVLKKDGEEAALAFL |
| | | SERGVSRGELPNFRPPAKTLVVAQSRPFEEFPIYRVSEAIQLYVYSLSVKE |
| | | LETVPSGSSTKKEHQRFFQDSSVPDFGYTSVQGLNKIFGLARGIYLGVITR |
| | | GENQLQKAKSKHEALNKKRRASGEAETEFDPTPYEYMTPERKLAKPPG |
| | | VNHSIMCYVDISVDEFDFRNPDGIVLPSEYAGYCREINTAIEKGTVDRLG |
| | | HLKGGPGYIPGHQRKESTTEGPKINFRKGRIRRSYTALYAKRDSRRVRQG |
| | | KLALPSYRHHMMRLNSNAESAILAVIFFGKDWVVFDLRGLLRNVRWRN |
| | | LFVDGSTPSTLLGMFGDPVIDPKRGVVAFCYKEQIVPVVSKSITKMVKAP |
| | | ELLNKLYLKSEDPLVLVAIDLGQTNPVGVGVYRVMNASLDYEVVTRFA |
| | | LESELLREIESYRQRTNAFEAQIRAETFDAMTSEEQEEITRVRAFSASKAK |
| | | ENVCHRFGMPVDAVDWATMGSNTIHIAKWVMRHGDPSLVEVLEYRKD |
| | | NEIKLDKNGVPKKVKLTDKRIANLTSIRLRFSQETSKHYNDTMWELRRK |
| | | HPVYQKLSKSKADFSRRVVNSIIRRVNHLVPRARIVFIIEDLKNLGKVFHG |
| | | SGKRELGWDSYFEPKSENRWFIQVLHKAFSETGKHKGYYIIECWPNWTS |
| | | CTCPKCSCCDSENRHGEVFRCLACGYTCNTDFGTAPDNLVKIATTGKGL |
| | | PGPKKRCKGSSKGKNPKIARSSETGVSVTESGAPKVKKSSPTQTSQSSSQ |
| | | SAP |
| CasΦ.15 | 60 | MIKPTVSQFLTPGFKLIRNHSRTAGLKLKNEGEEACKKFVRENEIPKDEC |
| | | PNFQGGPAIANIIAKSREFTEWEIYQSSLAIQEVIFTLPKDKLPEPILKEEW |
| | | RAQWLSEHGLDTVPYKEAAGLNLIIKNAVNTYKGVQVKVDNKNKNNL |
| | | AKINRKNEIAKLNGEQEISFEEIKAFDDKGYLLQKPSPNKSIYCYQSVSPK |
| | | PFITSKYHNVNLPEEYIGYYRKSNEPIVSPYQFDRLRIPIGEPGYVPKWQY |
| | | TFLSKKENKRRKLSKRIKNVSPILGIICIKKDWCVFDMRGLLRTNHWKKY |
| | | HKPTDSINDLFDYFTGDPVIDTKANVVRFRYKMENGIVNYKPVREKKGK |
| | | ELLENICDQNGSCKLATVDVGQNNPVAIGLFELKKVNGELTKTLISRHPT |
| | | PIDFCNKITAYRERYDKLESSIKLDAIKQLTSEQKIEVDNYNNNFTPQNTK |
| | | QIVCSKLNINPNDLPWDKMISGTHFISEKAQVSNKSEIYFTSTDKGKTKD |
| | | VMKSDYKWFQDYKPKLSKEVRDALSDIEWRLRRESLEFNKLSKSREQD |
| | | ARQLANWISSMCDVIGIENLVKKNNFFGGSGKREPGWDNFYKPKKENR |
| | | WWINAIHKALTELSQNKGKRVILLPAMRTSITCPKCKYCDSKNRNGEKF |
| | | NCLKCGIELNADIDVATENLATVAITAQSMPKPTCERSGDAKKPVRARK |
| | | AKAPEFHDKLAPSYTVVLREAV |
| CasΦ.16 | 61 | MSNKTTPPSPLSLLLRAHFPGLKFESQDYKIAGKKLRDGGPEAVISYLTG |
| | | KGQAKLKDVKPPAKAFVIAQSRPFIEWDLVRVSRQIQEKIFGIPATKGRP |
| | | KQDGLSETAFNEAVASLEVDGKSKLNEETRAAFYEVLGLDAPSLHAQA |
| | | QNALIKSAISIREGVLKKVENRNEKNLSKTKRRKEAGEEATFVEEKAHDE |
| | | RGYLIHPPGVNQTIPGYQAVVIKSCPSDFIGLPSGCLAKESAEALTDYLPH |
| | | DRMTIPKGQPGYVPEWQHPLLNRRKNRRRRDWYSASLNKPKATCSKRS |
| | | GTPNRKNSRTDQIQSGRFKGAIPVLMRFQDEWVIIDIRGLLRNARYRKLL |
| | | KEKSTIPDLLSLFTGDPSIDMRQGVCTFIYKAGQACSAKMVKTKNAPEIL |
| | | SELTKSGPVVLVSIDLGQTNPIAAKVSRVTQLSDGQLSHETLLRELLSNDS |
| | | SDGKEIARYRVASDRLRDKLANLAVERLSPEHKSEILRAKNDTPALCKA |
| | | RVCAALGLNPEMIAWDKMTPYTEFLATAYLEKGGDRKVATLKPKNRPE |
| | | MLRRDIKFKGTEGVRIEVSPEAAEAYREAQWDLQRTSPEYLRLSTWKQE |
| | | LTKRILNQLRHKAAKSSQCEVVVMAFEDLNIKMMHGNGKWADGGWD |
| | | AFFIKKRENRWFMQAFHKSLTELGAHKGVPTIEVTPHRTSITCTKCGHCD |
| | | KANRDGERFACQKCGFVAHADLEIATDNIERVALTGKPMPKPESERSGD |
| | | AKKSVGARKAAFKPEEDAEAAE |
| CasΦ.17 | 62 | MYSLEMADLKSEPSLLAKLLRDRFPGKYWLPKYWKLAEKKRLTGGEEA |
| | | ACEYMADKQLDSPPPNFRPPARCVILAKSRPFEDWPVHRVASKAQSFVI |
| | | GLSEQGFAALRAAPPSTADARRDWLRSHGASEDDLMALEAQLLETIMG |
| | | NAISLHGGVLKKIDNANVKAAKRLSGRNEARLNKGLQELPPEQEGSAYG |
| | | ADGLLVNPPGLNLNIYCRKSCCPKPVKNTARFVGHYPGYLRDSDSILISG |
| | | TMDRLTIIEGMPGHIPAWQREQGLVKPGGRRRRLSGSESNMRQKVDPST |
| | | GPRRSTRSGTVNRSNQRTGRNGDPLLVEIRMKEDWVLLDARGLLRNLR |
| | | WRESKRGLSCDHEDLSLSGLLALFSGDPVIDPVRNEVVFLYGEGIIPVRST |
| | | KPVGTRQSKKLLERQASMGPLTLISCDLGQTNLIAGRASAISLTHGSLGV |
| | | RSSVRIELDPEIIKSFERLRKDADRLETEILTAAKETLSDEQRGEVNSHEK |
| | | DSPQTAKASLCRELGLHPPSLPWGQMGPSTTFIADMLISHGRDDDAFLSH |
| | | GEFPTLEKRKKFDKRFCLESRPLLSSETRKALNESLWEVKRTSSEYARLS |
| | | QRKKEMARRAVNFVVEISRRKTGLSNVIVNIEDLNVRIFHGGGKQAPGW |
| | | DGFFRPKSENRWFIQAIHKAFSDLAAHHGIPVIESDPQRTSMTCPECGHC |
| | | DSKNRNGVRFLCKGCGASMDADFDAACRNLERVALTGKPMPKPSTSCE |
| | | RLLSATTGKVCSDHSLSHDAIEKAS |
| CasΦ.18 | 63 | MEKEITELTKIRREFPNKKFSSTDMKKAGKLLKAEGPDAVRDFLNSCQEI |
| | | IGDFKPPVKTNIVSISRPFEEWPVSMVGRAIQEYYFSLTKEELESVHPGTS |
| | | SEDHKSFFNITGLSNYNYTSVQGLNLIFKNAKAIYDGTLVKANNKNKKL |
| | | EKKFNEINHKRSLEGLPIITPDFEEPFDENGHLNNPPGINRNIYGYQGCAA |
| | | KVFVPSKHKMVSLPKEYEGYNRDPNLSLAGFRNRLEIPEGEPGHVPWFQ |
| | | RMDIPEGQIGHVNKIQRFNFVHGKNSGKVKFSDKTGRVKRYHHSKYKD |
| | | ATKPYKFLEESKKVSALDSILAIITIGDDWVVFDIRGLYRNVFYRELAQK |
| | | GLTAVQLLDLFTGDPVIDPKKGVVTFSYKEGVVPVFSQKIVPRFKSRDTL |
| | | EKLTSQGPVALLSVDLGQNEPVAARVCSLKNINDKITLDNSCRISFLDDY |
| | | KKQIKDYRDSLDELEIKIRLEAINSLETNQQVEIRDLDVFSADRAKANTV |
| | | DMFDIDPNLISWDSMSDARVSTQISDLYLKNGGDESRVYFEINNKRIKRS |
| | | DYNISQLVRPKLSDSTRKNLNDSIWKLKRTSEEYLKLSKRKLELSRAVVN |
| | | YTIRQSKLLSGINDIVIILEDLDVKKKFNGRGIRDIGWDNFFSSRKENRWFI |
| | | PAFHKTFSELSSNRGLCVIEVNPAWTSATCPDCGFCSKENRDGINFTCRK |
| | | CGVSYHADIDVATLNIARVAVLGKPMSGPADRERLGDTKKPRVARSRK |
| | | TMKRKDISNSTVEAMVTA |
| CasΦ.19 | 64 | MLVRTSTLVQDNKNSRSASRAFLKKPKMPKNKHIKEPTELAKLIRELFPG |
| | | QRFTRAINTQAGKILKHKGRDEVVEFLKNKGIDKEQFMDFRPPTKARIV |
| | | ATSGAIEEFSYLRVSMAIQECCFGKYKFPKEKVNGKLVLETVGLTKEELD |
| | | DFLPKKYYENKKSRDRFFLKTGICDYGYTYAQGLNEIFRNTRAIYEGVFT |
| | | KVNNRNEKRREKKDKYNEERRSKGLSEEPYDEDESATDESGHLINPPGV |
| | | NLNIWTCEGFCKGPYVTKLSGTPGYEVILPKVFDGYNRDPNEIISCGITDR |
| | | FAIPEGEPGHIPWHQRLEIPEGQPGYVPGHQRFADTGQNNSGKANPNKK |
| | | GRMRKYYGHGTKYTQPGEYQEVFRKGHREGNKRRYWEEDFRSEAHDC |
| | | ILYVIHIGDDWVVCDLRGPLRDAYRRGLVPKEGITTQELCNLFSGDPVID |
| | | PKHGVVTFCYKNGLVRAQKTISAGKKSRELLGALTSQGPIALIGVDLGQ |
| | | TEPVGARAFIVNQARGSLSLPTLKGSFLLTAENSSSWNVFKGEIKAYREA |
| | | IDDLAIRLKKEAVATLSVEQQTEIESYEAFSAEDAKQLACEKFGVDSSFIL |
| | | WEDMTPYHTGPATYYFAKQFLKKNGGNKSLIEYIPYQKKKSKKTPKAV |
| | | LRSDYNIACCVRPKLLPETRKALNEAIRIVQKNSDEYQRLSKRKLEFCRR |
| | | VVNYLVRKAKKLTGLERVIIAIEDLKSLEKFFTGSGKRDNGWSNFFRPKK |
| | | ENRWFIPAFHKAFSELAPNRGFYVIECNPARTSITDPDCGYCDGDNRDGI |
| | | KFECKKCGAKHHTDLDVAPLNIAIVAVTGRPMPKTVSNKSKRERSGGEK |
| | | SVGASRKRNHRKSKANQEMLDATSSAAE |
| CasΦ.20 | 65 | MPKIKKPTEISLLRKEVFPDLHFAKDRMRAASLVLKNEGREAAIEYLRVN |
| | | HEDKPPNFMPPAKTPYVALSRPLEQWPIAQASIAIQKYIFGLTKDEFSATK |
| | | KLLYGDKSTPNTESRKRWFEVTGVPNFGYMSAQGLNAIFSGALARYEG |
| | | VVQKVENRNKKRFEKLSEKNQLLIEEGQPVKDYVPDTAYHTPETLQKLA |
| | | ENNHVRVEDLGDMIDRLVHPPGIHRSIYGYQQVPPFAYDPDNPKGIILPK |
| | | AYAGYTRKPHDIIEAMPNRLNIPEGQAGYIPEHQRDKLKKGGRVKRLRT |
| | | TRVRVDATETVRAKAEALNAEKARLRGKEAILAVFQIEEDWALIDMRG |
| | | LLRNVYMRKLIAAGELTPTTLLGYFTETLTLDPRRTEATFCYHLRSEGAL |
| | | HAEYVRHGKNTRELLLDLTKDNEKIALVTIDLGQRNPLAAAIFRVGRDA |
| | | SGDLTENSLEPVSRMLLPQAYLDQIKAYRDAYDSFRQNIWDTALASLTP |
| | | EQQRQILAYEAYTPDDSKENVLRLLLGGNVMPDDLPWEDMTKNTHYIS |
| | | DRYLADGGDPSKVWFVPGPRKRKKNAPPLKKPPKPRELVKRSDHNISHL |
| | | SEFRPQLLKETRDAFEKAKIDTERGHVGYQKLSTRKDQLCKEILNWLEA |
| | | EAVRLTRCKTMVLGLEDLNGPFFNQGKGKVRGWVSFFRQKQENRWIV |
| | | NGFRKNALARAHDKGKYILELWPSWTSQTCPKCKHVHADNRHGDDFV |
| | | CLQCGARLHADAEVATWNLAVVAIQGHSLPGPVREKSNDRKKSGSARK |
| | | SKKANESGKVVGAWAAQATPKRATSKKETGTARNPVYNPLETQASCPA |
| | | P |
| CasΦ.21 | 66 | MTPSPQIARLVETPLAAALKAHHPGKKFRSDYLKKAGKILKDQGVEAA |
| | | MAHLDGKDQAEPPNFKPPAKCRIVARSREFSEWPIVKASVEIQKYIYGLT |
| | | LEERKACDPGKSSASHKAWFAKTGVNTFGYSSVQGFNLIFGHTLGRYDG |
| | | VLVKTENLNKKRAEKNERFRAKALAEGRAEPVCPPLVTATNDTGQDVT |
| | | LEDGRVVRPGQLLQPPGINPNIYAYQQVSPKAYVPGIIELPEEFQGYSRDP |
| | | NAVILPLVPRDRLSIPKGQPGYVPEPHREGLTGRKDRRMRRYYETERGT |
| | | KLKRPPLTAKGRADKANEALLVVVRIDSDWVVMDVRGLLRNARWRRL |
| | | VSKEGITLNGLLDLFTGDPVLNPKDCSVSRDTGDPVNDPRHGVVTFCYK |
| | | LGVVDVCSKDRPIKGFRTKEVLERLTSSGTVGMVSIDLGQTNPVAAAVS |
| | | RVTKGLQAETLETFTLPDDLLGKVRAYRAKTDRMEEGFRRNALRKLTA |
| | | EQQAEITRYNDATEQQAKALVCSTYGIGPEEVPWERMTSNTTYISDHILD |
| | | HGGDPDTVFFMATKRGQNKPTLHKRKDKAWGQKFRPAISVETRLARQA |
| | | AEWELRRASLEFQKLSVWKTELCRQAVNYVMERTKKRTQCDVIIPVIED |
| | | LPVPLFHGSGKRDPGWANFFVHKRENRWFIDGLHKAFSELGKHRGIYVF |
| | | EVCPQRTSITCPKCGHCDPDNRDGEKFVCLSCQATLNADLDVATTNLVR |
| | | VALTGKVMPRSERSGDAQTPGPARKARTGKIKGSKPTSAPQGATQTDA |
| | | KAHLSQTGV |
| CasΦ.22 | 67 | MTPSPQIARLVETPLAAALKAHHPGKKFRSDYLKKAGKILKDQGVEAA |
| | | MAHLDGKDQAEPPNFKPPAKCRIVARSREFSEWPIVKASVEIQKYIYGLT |
| | | LEERKACDPGKSSASHKAWFAKTGVNTFGYSSVQGFNLIFGHTLGRYDG |
| | | VLVKTENLNKKRAEKNERFRAKALAEGRAEPVCPPLVTATNDTGQDVT |
| | | LEDGRVVRPGQLLQPPGINPNIYAYQQVSPKAYVPGIIELPEEFQGYSRDP |
| | | NAVILPLVPRDRLSIPKGQPGYVPEPHREGLTGRKDRRMRRYYETERGT |
| | | KLKRPPLTAKGRADKANEALLVVVRIDSDWVVMDVRGLLRNARWRRL |
| | | VSKEGITLNGLLDLFTGDPVLNPKDCSVSRDTGDPVNDPRHGVVTFCYK |
| | | LGVVDVCSKDRPIKGFRTKEVLERLTSSGTVGMVSIDLGQTNPVAAAVS |
| | | RVTKGLQAETLETFTLPDDLLGKVRAYRAKTDRMEEGFRRNALRKLTA |
| | | EQQAEITRYNDATEQQAKALVCSTYGIGPEEVPWERMTSNTTYISDHILD |
| | | HGGDPDTVFFMATKRGQNKPTLHKRKDKAWGQKFRPAISVETRLARQA |
| | | AEWELRRASLEFQKLSVWKTELCRQAVNYVMERTKKRTQCDVIIPVIED |
| | | LPVPLFHGSGKRDPGWANFFVHKRENRWFIDGLHKAFSELGKHRGIYVF |
| | | EVCPQRTSITCPKCGHCDPDNRDGEKFVCLSCQATLHADLDVATTNLVR |
| | | VALTGKVMPRSERSGDAQTPGPARKARTGKIKGSKPTSAPQGATQTDA |
| | | KAHLSQTGV |
| CasΦ.23 | 68 | MKTEKPKTALTLLREEVFPGKKYRLDVLKEAGKKLSTKGREATIEFLTG |
| | | KDEERPQNFQPPAKTSIVAQSRPFDQWPIVQVSLAVQKYIYGLTQSEFEA |
| | | NKKALYGETGKAISTESRRAWFEATGVDNFGFTAAQGINPIFSQAVARY |
| | | EGVIKKVENRNEKKLKKLTKKNLLRLESGEEIEDFEPEATFNEEGRLLQP |
| | | PGANPNIYCYQQISPRIYDPSDPKGVILPQIYAGYDRKPEDIISAGVPNRLA |
| | | IPEGQPGYIPEHQRAGLKTQGRIRCRASVEAKARAAILAVVHLGEDWVV |
| | | LDLRGLLRNVYWRKLASPGTLTLKGLLDFFTGGPVLDARRGIATFSYTL |
| | | KSAAAVHAENTYKGKGTREVLLKLTENNSVALVTVDLGQRNPLAAMIA |
| | | RVSRTSQGDLTYPESVEPLTRLFLPDPFLEEVRKYRSSYDALRLSIREAAI |
| | | ASLTPEQQAEIRYIEKFSAGDAKKNVAEVFGIDPTQLPWDAMTPRTTYIS |
| | | DLFLRMGGDRSRVFFEVPPKKAKKAPKKPPKKPAGPRIVKRTDGMIARL |
| | | REIRPRLSAETNKAFQEARWEGERSNVAFQKLSVRRKQFARTVVNHLVQ |
| | | TAQKMSRCDTVVLGIEDLNVPFFHGRGKYQPGWEGFFRQKKENRWLIN |
| | | DMHKALSERGPHRGGYVLELTPFWTSLRCPKCGHTDSANRDGDDFVCV |
| | | KCGAKLHSDLEVATANLALVAITGQSIPRPPREQSSGKKSTGTARMKKT |
| | | SGETQGKGSKACVSEALNKIEQGTARDPVYNPLNSQVSCPAP |
| CasΦ.24 | 69 | VYNPDMKKPNNIRRIREEHFEGLCFGKDVLTKAGKIYEKDGEEAAIDFL |
| | | MGKDEEDPPNFKPPAKTTIVAQSRPFDQWPIYQVSQAVQERVFAYTEEE |
| | | FNASKEALFSGDISSKSRDFWFKTNNISDQGIGAQGLNTILSHAFSRYSGV |
| | | IKKVENRNKKRLKKLSKKNQLKIEEGLEILEFKPDSAFNENGLLAQPPGI |
| | | NPNIYGYQAVTPFVFDPDNPGDVILPKQYEGYSRKPDDIIEKGPSRLDIPK |
| | | GQPGYVPEHQRKNLKKKGRVRLYRRTPPKTKALASILAVLQIGKDWVL |
| | | FDMRGLLRSVYMREAATPGQISAKDLLDTFTGCPVLNTRTGEFTFCYKL |
| | | RSEGALHARKIYTKGETRTLLTSLTSENNTIALVTVDLGQRNPAAIMISRL |
| | | SRKEELSEKDIQPVSRRLLPDRYLNELKRYRDAYDAFRQEVRDEAFTSLC |
| | | PEHQEQVQQYEALTPEKAKNLVLKHFFGTHDPDLPWDDMTSNTHYIAN |
| | | LYLERGGDPSKVFFTRPLKKDSKSKKPRKPTKRTDASISRLPEIRPKMPED |
| | | ARKAFEKAKWEIYTGHEKFPKLAKRVNQLCREIANWIEKEAKRLTLCDT |
| | | VVVGIEDLSLPPKRGKGKFQETWQGFFRQKFENRWVIDTLKKAIQNRAH |
| | | DKGKYVLGLAPYWTSQRCPACGFIHKSNRNGDHFKCLKCEALFHADSE |
| | | VATWNLALVAVLGKGITNPDSKKPSGQKKTGTTRKKQIKGKNKGKETV |
| | | NVPPTTQEVEDIIAFFEKDDETVRNPVYKPTGT |
| CasΦ.25 | 70 | MKKPNNIRRIREEHFEGLCFGKDVLTKAGKIYEKDGEEAAIDFLMGKDE |
| | | EDPPNFKPPAKTTIVAQSRPFDQWPIYQVSQAVQERVFAYTEEEFNASKE |
| | | ALFSGDISSKSRDFWFKTNNISDQGIGAQGLNTILSHAFSRYSGVIKKVEN |
| | | RNKKRLKKLSKKNQLKIEEGLEILEFKPDSAFNENGLLAQPPGINPNIYGY |
| | | QAVTPFVFDPDNPGDVILPKQYEGYSRKPDDIIEKGPSRLDIPKGQPGYVP |
| | | EHQRKNLKKKGRVRLYRRTPPKTKALASILAVLQIGKDWVLFDMRGLL |
| | | RSVYMREAATPGQISAKDLLDTFTGCPVLNTRTGEFTFCYKLRSEGALH |
| | | ARKIYTKGETRTLLTSLTSENNTIALVTVDLGQRNPAAIMISRLSRKEELS |
| | | EKDIQPVSRRLLPDRYLNELKRYRDAYDAFRQEVRDEAFTSLCPEHQEQ |
| | | VQQYEALTPEKAKNLVLKHFFGTHDPDLPWDDMTSNTHYIANLYLERG |
| | | GDPSKVFFTRPLKKDSKSKKPRKPTKRTDASISRLPEIRPKMPEDARKAFE |
| | | KAKWEIYTGHEKFPKLAKRVNQLCREIANWIEKEAKRLTLCDTVVVGIE |
| | | DLSLPPKRGKGKFQETWQGFFRQKFENRWVIDTLKKAIQNRAHDKGKY |
| | | VLGLAPYWTSQRCPACGFIHKSNRNGDHFKCLKCEALFHADSEVATWN |
| | | LALVAVLGKGITNPDSKKPSGQKKTGTTRKKQIKGKNKGKETVNVPPTT |
| | | QEVEDIIAFFEKDDETVRNPVYKPTGT |
| CasΦ.26 | 71 | VIKTHFPAGRFRKDHQKTAGKKLKHEGEEACVEYLRNKVSDYPPNFKPP |
| | | AKGTIVAQSRPFSEWPIVRASEAIQKYVYGLTVAELDVFSPGTSKPSHAE |
| | | WFAKTGVENYGYRQVQGLNTIFQNTVNRFKGVLKKVENRNKKSLKRQ |
| | | EGANRRRVEEGLPEVPVTVESATDDEGRLLQPPGVNPSIYGYQGVAPRV |
| | | CTDLQGFSGMSVDFAGYRRDPDAVLVESLPEGRLSIPKGERGYVPEWQR |
| | | DPERNKFPLREGSRRQRKWYSNACHKPKPGRTSKYDPEALKKASAKDA |
| | | LLVSISIGEDWAIIDVRGLLRDARRRGFTPEEGLSLNSLLGLFTEYPVFDV |
| | | QRGLITFTYKLGQVDVHSRKTVPTFRSRALLESLVAKEEIALVSVDLGQT |
| | | NPASMKVSRVRAQEGALVAEPVHRMFLSDVLLGELSSYRKRMDAFEDA |
| | | IRAQAFETMTPEQQAEITRVCDVSVEVARRRVCEKYSISPQDVPWGEMT |
| | | GHSTFIVDAVLRKGGDESLVYFKNKEGETLKFRDLRISRMEGVRPRLTK |
| | | DTRDALNKAVLDLKRAHPTFAKLAKQKLELARRCVNFIEREAKRYTQC |
| | | ERVVFVIEDLNVGFFHGKGKRDRGWDAFFTAKKENRWVIQALHKAFSD |
| | | LGLHRGSYVIEVTPQRTSMTCPRCGHCDKGNRNGEKFVCLQCGATLHA |
| | | DLEVATDNIERVALTGKAMPKPPVRERSGDVQKAGTARKARKPLKPKQ |
| | | KTEPSVQEGSSDDGVDKSPGDASRNPVYNPSDTLSI |
| CasΦ.27 | 72 | MAKAKTLAALLRELLPGQHLAPHHRWVANKLLMTSGDAAAFVIGKSVS |
| | | DPVRGSFRKDVITKAGRIFKKDGPDAAAAFLDGKWEDRPPNFQPPAKAA |
| | | IVAISRSFDEWPIVKVSCAIQQYLYALPVQEFESSVPEARAQAHAAWFQD |
| | | TGVDDCNFKSTQGLNAIFNHGKRTYEGVLKKAQNRNDKKNLRLERINA |
| | | KRAEAGQAPLVAGPDESPTDDAGCLLHPPGINANIYCYQQVSPRPYEQS |
| | | CGIQLPPEYAGYNRLSNVAIPPMPNRLDIPQGQPGYVPEHHRHGIKKFGR |
| | | VRKRYGVVPGRNRDADGKRTRQVLTEAGAAAKARDSVLAVIRIGDDW |
| | | TVVDLRGLLRNAQWRKLVPDGGITVQGLLDLFTGDPVIDPRRGVVTFIY |
| | | KADSVGIHSEKVCRGKQSKNLLERLCAMPEKSSTRLDCARQAVALVSV |
| | | DLGQRNPVAARFSRVSLAEGQLQAQLVSAQFLDDAMVAMIRSYREEYD |
| | | RFESLVREQAKAALSPEQLSEIVRHEADSAESVKSCVCAKFGIDPAGLSW |
| | | DKMTSGTWRIADHVQAAGGDVEWFFFKTCGKGKEIKTVRRSDFNVAK |
| | | QFRLRLSPETRKDWNDAIWELKRGNPAYVSFSKRKSEFARRVVNDLVH |
| | | RARRAVRCDEVVFAIEDLNISFFHGKGQRQMGWDAFFEVKQENRWFIQ |
| | | ALHKAFVERATHKGGYVLEVAPARTSTTCPECRHCDPESRRGEQFCCIK |
| | | CRHTCHADLEVATFNIEQVALTGVSLPKRLSSTLL |
| CasΦ.28 | 73 | MSKEKTPPSAYAILKAKHFPDLDFEKKHKMMAGRMFKNGASEQEVVQ |
| | | YLQGKGSESLMDVKPPAKSPILAQSRPFDEWEMVRTSRLIQETIFGIPKRG |
| | | SIPKRDGLSETQFNELVASLEVGGKPMLNKQTRAIFYGLLGIKPPTFHAM |
| | | AQNILIDLAINIRKGVLKKVDNLNEKNRKKVKRIRDAGEQDVMVPAEVT |
| | | AHDDRGYLNHPPGVNPTIPGYQGVVIPFPEGFEGLPSGMTPVDWSHVLV |
| | | DYLPHDRLSIPKGSPGYIPEWQRPLLNRHKGRRHRSWYANSLNKPRKSR |
| | | TEEAKDRQNAGKRTALIEAERLKGVLPVLMRFKEDWLIIDARGLLRNAR |
| | | YRGVLPEGSTLGNLIDLFSDSPRVDTRRGICTFLYRKGRAYSTKPVKRKE |
| | | SKETLLKLTEKSTIALVSIDLGQTNPLTAKLSKVRQVDGCLVAEPVLRKLI |
| | | DNASEDGKEIARYRVAHDLLRARILEDAIDLLGIYKDEVVRARSDTPDLC |
| | | KERVCRFLGLDSQAIDWDRMTPYTDFIAQAFVAKGGDPKVVTIKPNGKP |
| | | KMFRKDRSIKNMKGIRLDISKEASSAYREAQWAIQRESPDFQRLAVWQS |
| | | QLTKRIVNQLVAWAKKCTQCDTVVLAFEDLNIGMMHGSGKWANGGW |
| | | NALFLHKQENRWFMQAFHKALTELSAHKGIPTIEVLPHRTSITCTQCGHC |
| | | HPGNRDGERFKCLKCEFLANTDLEIATDNIERVALTGLPMPKGERSSAKR |
| | | KPGGTRKTKKSKHSGNSPLAAE |
| CasΦ.29 | 74 | MEKAGPTSPLSVLIHKNFEGCRFQIDHLKIAGRKLAREGEAAAIEYLLDK |
| | | KCEGLPPNFQPPAKGNVIAQSRPFTEWAPYRASVAIQKYIYSLSVDERKV |
| | | CDPGSSSDSHEKWFKQTGVQNYGYTHVQGLNLIFKHALARYDGVLKKV |
| | | DNRNEKNRKKAERVNSFRREEGLPEEVFEEEKATDETGHLLQPPGVNHS |
| | | IYCYQSVRPKPFNPRKPGGISLPEAYSGYSLKPQDELPIGSLDRLSIPPGQP |
| | | GYVPEWQRSQLTTQKHRRKRSWYSAQKWKPRTGRTSTFDPDRLNCAR |
| | | AQGAILAVVRIHEDWVVFDVRGLLRNALWRELAGKGLTVRDLLDFFTG |
| | | DPVVDTKRGVVTFTYKLGKVDVHSLRTVRGKRSKKVLEDLTLSSDVGL |
| | | VTIDLGQTNVLAADYSKVTRSENGELLAVPLSKSFLPKHLLHEVTAYRTS |
| | | YDQMEEGFRRKALLTLTEDQQVEVTLVRDFSVESSKTKLLQLGVDVTSL |
| | | PWEKMSSNTTYISDQLLQQGADPASLFFDGERDGKPCRHKKKDRTWAY |
| | | LVRPKVSPETRKALNEALWALKNTSPEFESLSKRKIQFSRRCMNYLLNE |
| | | AKRISGCGQVVFVIEDLNVRVHHGRGKRAIGWDNFFKPKRENRWFMQA |
| | | LHKAASELAIHRGMHIIEACPARSSITCPKCGHCDPENRCSSDREKFLCVK |
| | | CGAAFHADLEVATFNLRKVALTGTALPKSIDHSRDGLIPKGARNRKLKE |
| | | PQANDEKACA |
| CasΦ.30 | 75 | MKEQSPLSSVLKSNFPGKKFLSADIRVAGRKLAQLGEAAAVEYLSPRQR |
| | | DSVPNFRPPAFCTVVAKSRPFEEWPIYKASVLLQEQIYGMTGQEFEERCG |
| | | SIPTSLSGLRQWASSVGLGAAMEGLHVQGMNLMVKNAINRYKGVLVK |
| | | VENRNKKLVEANEAKNSSREERGLPPLRPPELGSAFGPDGRLVNPPGIDK |
| | | SIRLYQGVSPVPVVKTTGRPTVHRLDIPAGEKGHVPLWQREAGLVKEGP |
| | | RRRRMWYSNSNLKRSRKDRSAEASEARKADSVVVRVSVKEDWVDIDV |
| | | RGLLRNVAWRGIERAGESTEDLLSLFSGDPVVDPSRDSVVFLYKEGVVD |
| | | VLSKKVVGAGKSRKQLEKMVSEGPVALVSCDLGQTNYVAARVSVLDES |
| | | LSPVRSFRVDPREFPSADGSQGVVGSLDRIRADSDRLEAKLLSEAEASLP |
| | | EPVRAEIEFLRSERPSAVAGRLCLKLGIDPRSIPWEKMGSTTSFISEALSAK |
| | | GSPLALHDGAPIKDSRFAHAARGRLSPESRKALNEALWERKSSSREYGVI |
| | | SRRKSEASRRMANAVLSESRRLTGLAVVAVNLEDLNMVSKFFHGRGKR |
| | | APGWAGFFTPKMENRWFIRSIHKAMCDLSKHRGITVIESRPERTSISCPEC |
| | | GHCDPENRSGERFSCKSCGVSLHADFEVATRNLERVALTGKPMPRRENL |
| | | HSPEGATASRKTRKKPREATASTFLDLRSVLSSAENEGSGPAARAG |
| CasΦ.31 | 76 | MLPPSNKIGKSMSLKEFINKRNFKSSIIKQAGKILKKEGEEAVKKYLDDN |
| | | YVEGYKKRDFPITAKCNIVASNRKIEDFDISKFSSFIQNYVFNLNKDNFEE |
| | | FSKIKYNRKSFDELYKKIANEIGLEKPNYENIQGEIAVIRNAINIYNGVLK |
| | | KVENRNKKIQEKNQSKDPPKLLSAFDDNGFLAERPGINETIYGYQSVRLR |
| | | HLDVEKDKDIIVQLPDIYQKYNKKSTDKISVKKRLNKYNVDEYGKLISK |
| | | RRKERINKDDAILCVSNFGDDWIIFDARGLLRQTYRYKLKKKGLCIKDLL |
| | | NLFTGDPIINPTKTDLKEALSLSFKDGIINNRTLKVKNYKKCPELISELIRD |
| | | KGKVAMISIDLGQTNPISYRLSKFTANNVAYIENGVISEDDIVKMKKWRE |
| | | KSDKLENLIKEEAIASLSDDEQREVRLYENDIADNTKKKILEKFNIREEDL |
| | | DFSKMSNNTYFIRDCLKNKNIDESEFTFEKNGKKLDPTDACFAREYKNK |
| | | LSELTRKKINEKIWEIKKNSKEYHKISIYKKETIRYIVNKLIKQSKEKSECD |
| | | DIIVNIEKLQIGGNFFGGRGKRDPGWNNFFLPKEENRWFINACHKAFSEL |
| | | APHKGIIVIESDPAYTSQTCPKCENCDKENRNGEKFKCKKCNYEANADID |
| | | VATENLEKIAKNGRRLIKNFDQLGERLPGAEMPGGARKRKPSKSLPKNG |
| | | RGAGVGSEPELINQSPSQVIA |
| CasΦ.32 | 77 | VPDKKETPLVALCKKSFPGLRFKKHDSRQAGRILKSKGEGAAVAFLEGK |
| | | GGTTQPNFKPPVKCNIVAMSRPLEEWPIYKASVVIQKYVYAQSYEEFKA |
| | | TDPGKSEAGLRAWLKATRVDTDGYFNVQGLNLIFQNARATYEGVLKKV |
| | | ENRNSKKVAKIEQRNEHRAERGLPLLTLDEPETALDETGHLRHRPGINCS |
| | | VFGYQHMKLKPYVPGSIPGVTGYSRDPSTPIAACGVDRLEIPEGQPGYVP |
| | | PWDRENLSVKKHRRKRASWARSRGGAIDDNMLLAVVRVADDWALLD |
| | | LRGLLRNTQYRKLLDRSVPVTIESLLNLVTNDPTLSVVKKPGKPVRYTAT |
| | | LIYKQGVVPVVKAKVVKGSYVSKMLDDTTETFSLVGVDLGVNNLIAAN |
| | | ALRIRPGKCVERLQAFTLPEQTVEDFFRFRKAYDKHQENLRLAAVRSLT |
| | | AEQQAEVLALDTFGPEQAKMQVCGHLGLSVDEVPWDKVNSRSSILSDL |
| | | AKERGVDDTLYMFPFFKGKGKKRKTEIRKRWDVNWAQHFRPQLTSETR |
| | | KALNEAKWEAERNSSKYHQLSIRKKELSRHCVNYVIRTAEKRAQCGKVI |
| | | VAVEDLHHSFRRGGKGSRKSGWGGFFAAKQEGRWLMDALFGAFCDLA |
| | | VHRGYRVIKVDPYNTSRTCPECGHCDKANRDRVNREAFICVCCGYRGN |
| | | ADIDVAAYNIAMVAITGVSLRKAARASVASTPLESLAAE |
| CasΦ.33 | 78 | MSKTKELNDYQEALARRLPGVRHQKSVRRAARLVYDRQGEDAMVAFL |
| | | DGKEVDEPYTLQPPAKCHILAVSRPIEEWPIARVTMAVQEHVYALPVHE |
| | | VEKSRPETTEGSRSAWFKNSGVSNHGVTHAQTLNAILKNAYNVYNGVI |
| | | KKVENRNAKKRDSLAAKNKSRERKGLPHFKADPPELATDEQGYLLQPP |
| | | SPNSSVYLVQQHLRTPQIDLPSGYTGPVVDPRSPIPSLIPIDRLAIPPGQPG |
| | | YVPLHDREKLTSNKHRRMKLPKSLRAQGALPVCFRVFDDWAVVDGRG |
| | | LLRHAQYRRLAPKNVSIAELLELYTGDPVIDIKRNLMTFRFAEAVVEVTA |
| | | RKIVEKYHNKYLLKLTEPKGKPVREIGLVSIDLNVQRLIALAIYRVHQTG |
| | | ESQLALSPCLHREILPAKGLGDFDKYKSKFNQLTEEILTAAVQTLTSAQQ |
| | | EEYQRYVEESSHEAKADLCLKYSITPHELAWDKMTSSTQYISRWLRDHG |
| | | WNASDFTQITKGRKKVERLWSDSRWAQELKPKLSNETRRKLEDAKHDL |
| | | QRANPEWQRLAKRKQEYSRHLANTVLSMAREYTACETVVIAIENLPMK |
| | | GGFVDGNGSRESGWDNFFTHKKENRWMIKDIHKALSDLAPNRGVHVLE |
| | | VNPQYTSQTCPECGHRDKANRDPIQRERFCCTHCGAQRHADLEVATHNI |
| | | AMVATTGKSLTGKSLAPQRLQEAAE |
| CasΦ.41 | 79 | VLLSDRIQYTDPSAPIPAMTVVDRRKIKKGEPGYVPPFMRKNLSTNKHRR |
| | | MRLSRGQKEACALPVGLRLPDGKDGWDFIIFDGRALLRACRRLRLEVTS |
| | | MDDVLDKFTGDPRIQLSPAGETIVTCMLKPQHTGVIQQKLITGKMKDRL |
| | | VQLTAEAPIAMLTVDLGEHNLVACGAYTVGQRRGKLQSERLEAFLLPEK |
| | | VLADFEGYRRDSDEHSETLRHEALKALSKRQQREVLDMLRTGADQARE |
| | | SLCYKYGLDLQALPWDKMSSNSTFIAQHLMSLGFGESATHVRYRPKRK |
| | | ASERTILKYDSRFAAEEKIKLTDETRRAWNEAIWECQRASQEFRCLSVRK |
| | | LQLARAAVNWTLTQAKQRSRCPRVVVVVEDLNVRFMHGGGKRQEGW |
| | | AGFFKARSEKRWFIQALHKAYTELPTNRGIHVMEVNPARTSITCTKCGY |
| | | CDPENRYGEDFHCRNPKCKVRGGHVANADLDIATENLARVALSGPMPK |
| | | APKLK |
| CasΦ.34 | 80 | MTPSFGYQMIIVTPIHHASGAWATLRLLFLNPKTSGVMLGMTKTKSAFA |
| | | LMREEVFPGLLFKSADLKMAGRKFAKEGREAAIEYLRGKDEERPANFKP |
| | | PAKGDIIAQSRPFDQWPIVQVSQAIQKYIFGLTKAEFDATKTLLYGEGNH |
| | | PTTESRRRWFEATGVPDFGFTSAQGLNAIFSSALARYEGVIQKVENRNEK |
| | | RLKKLSEKNQRLVEEGHAVEAYVPETAFHTLESLKALSEKSLVPLDDLM |
| | | DKIDRLAQPPGINPCLYGYQQVAPYIYDPENPRGVVLPDLYLGYCRKPD |
| | | DPITACPNRLDIPKGQPGYIPEHQRGQLKKHGRVRRFRYTNPQAKARAK |
| | | AQTAILAVLRIDEDWVVMDLRGLLRNVYFREVAAPGELTARTLLDTFTG |
| | | CPVLNLRSNVVTFCYDIESKGALHAEYVRKGWATRNKLLDLTKDGQSV |
| | | ALLSVDLGQRHPVAVMISRLKRDDKGDLSEKSIQVVSRTFADQYVDKLK |
| | | RYRVQYDALRKEIYDAALVSLPPEQQAEIRAYEAFAPGDAKANVLSVMF |
| | | QGEVSPDELPWDKMNTNTHYISDLYLRRGGDPSRVFFVPQPSTPKKNAK |
| | | KPPAPRKPVKRTDENVSHMPEFRPHLSNETREAFQKAKWTMERGNVRY |
| | | AQLSRFLNQIVREANNWLVSEAKKLTQCQTVVWAIEDLHVPFFHGKGK |
| | | YHETWDGFFRQKKEDRWFVNVFHKAISERAPNKGEYVMEVAPYRTSQR |
| | | CPVCGFVDADNRHGDHFKCLRCGVELHADLEVATWNIALVAVQGHGIA |
| | | GPPREQSCGGETAGTARKGKNIKKNKGLADAVTVEAQDSEGGSKKDAG |
| | | TARNPVYIPSESQVNCPAP |
| CasΦ.35 | 81 | MKPKTPKPPKTPVAALIDKHFPGKRFRASYLKSVGKKLKNQGEDVAVRF |
| | | LTGKDEERPPNFQPPAKSNIVAQSRPIEEWPIHKVSVAVQEYVYGLTVAE |
| | | KEACSDAGESSSSHAAWFAKTGVENFGYTSVQGLNKIFPPTFNRFDGVIK |
| | | KVENRNEKKRQKATRINEAKRNKGQSEDPPEAEVKATDDAGYLLQPPGI |
| | | NHSVYGYQSITLCPYTAEKFPTIKLPEEYAGYHSNPDAPIPAGVPDRLAIP |
| | | EGQPGHVPEEHRAGLSTKKHRRVRQWYAMANWKPKPKRTSKPDYDRL |
| | | AKARAQGALLIVIRIDEDWVVVDARGLLRNVRWRSLGKREITPNELLDL |
| | | FTGDPVLDLKRGVVTFTYAEGVVNVCSRSTTKGKQTKVLLDAMTAPRD |
| | | GKKRQIGMVAVDLGQTNPIAAEYSRVGKNAAGTLEATPLSRSTLPDELL |
| | | REIALYRKAHDRLEAQLREEAVLKLTAEQQAENARYVETSEEGAKLAL |
| | | ANLGVDTSTLPWDAMTGWSTCISDHLINHGGDTSAVFFQTIRKGTKKLE |
| | | TIKRKDSSWADIVRPRLTKETREALNDFLWELKRSHEGYEKLSKRLEEL |
| | | ARRAVNHVVQEVKWLTQCQDIVIVIEDLNVRNFHGGGKRGGGWSNFFT |
| | | VKKENRWFMQALHKAFSDLAAHRGIPVLEVYPARTSITCLGCGHCDPEN |
| | | RDGEAFVCQQCGATFHADLEVATRNIARVALTGEAMPKAPAREQPGGA |
| | | KKRGTSRRRKLTEVAVKSAEPTIHQAKNQQLNGTSRDPVYKGSELPAL |
| CasΦ.43 | 82 | MSEITDLLKANFKGKTFKSADMRMAGRILKKSGAQAVIKYLSDKGAVD |
| | | PPDFRPPAKCNIIAQSRPFDEWPICKASMAIQQHIYGLTKNEFDESSPGTSS |
| | | ASHEQWFAKTGVDTHGFTHVQGLNLIFQHAKKRYEGVIKKVENYNEKE |
| | | RKKFEGINERRSKEGMPLLEPRLRTAFGDDGKFAEKPGVNPSIYLYQQTS |
| | | PRPYDKTKHPYVHAPFELKEITTIPTQDDRLKIPFGAPGHVPEKHRSQLS |
| | | MAKHKRRRAWYALSQNKPRPPKDGSKGRRSVRDLADLKAASLADAIPL |
| | | VSRVGFDWVVIDGRGLLRNLRWRKLAHEGMTVEEMLGFFSGDPVIDPR |
| | | RNVATFIYKAEHATVKSRKPIGGAKRAREELLKATASSDGVIRQVGLISV |
| | | DLGQTNPVAYEISRMHQANGELVAEHLEYGLLNDEQVNSIQRYRAAWD |
| | | SMNESFRQKAIESLSMEAQDEIMQASTGAAKRTREAVLTMFGPNATLPW |
| | | SRMSSNTTCISDALIEVGKEEETNFVTSNGPRKRTDAQWAAYLRPRVNP |
| | | ETRALLNQAVWDLMKRSDEYERLSKRKLEMARQCVNFVVARAEKLTQ |
| | | CNNIGIVLENLVVRNFHGSGRRESGWEGFFEPKRENRWFMQVLHKAFSD |
| | | LAQHRGVMVFEVHPAYSSQTCPACRYVDPKNRSSEDRERFKCLKCGRSF |
| | | NADREVATFNIREIARTGVGLPKPDCERSRGVQTTGTARNPGRSLKSNK |
| | | NPSEPKRVLQSKTRKKITSTETQNEPLATDLKT |
| CasΦ.44 | 83 | MTPKTESPLSALCKKHFPGKRFRTNYLKDAGKILKKHGEDAVVAFLSDK |
| | | QEDEPANFCPPAKVHILAQSRPFEDWPINLASKAIQTYVYGLTADERKTC |
| | | EPGTSKESHDRWFKETGVDHHGFTSVQGLNLIFKHTLNRYDGVIKKVET |
| | | RNEKRRSSVVRINEKKAAEGLPLIAAEAEETAFGEDGRLLQPPGVNHSIY |
| | | CFQQVSPQPYSSKKHPQVVLPHAVQGVDPDAPIPVGRPNRLDIPKGQPG |
| | | YVPEWQRPHLSMKCKRVRMWYARANWRRKPGRRSVLNEARLKEASA |
| | | KGALPIVLVIGDDWLVMDARGLLRSVFWRRVAKPGLSLSELLNVTPTGL |
| | | FSGDPVIDPKRGLVTFTSKLGVVAVHSRKPTRGKKSKDLLLKMTKPTDD |
| | | GMPRHVGMVAIDLGQTNPVAAEYSRVVQSDAGTLKQEPVSRGVLPDDL |
| | | LKDVARYRRAYDLTEESIRQEAIALLSEGHRAEVTKLDQTTANETKRLL |
| | | VDRGVSESLPWEKMSSNTTYISDCLVALGKTDDVFFVPKAKKGKKETGI |
| | | AVKRKDHGWSKLLRPRTSPEARKALNENQWAVKRASPEYERLSRRKLE |
| | | LGRRCVNHIIQETKRWTQCEDIVVVLEDLNVGFFHGSGKRPDGWDNFFV |
| | | SKRENRWFIQVLHKAFGDLATHRGTHVIEVHPARTSITCIKCGHCDAGN |
| | | RDGESFVCLASACGDRRHADLEVATRNVARVAITGERMPPSEQARDVQ |
| | | KAGGARKRKPSARNVKSSYPAVEPAPASP |
| CasΦ.36 | 84 | MSDNKMKKLSKEEKPLTPLQILIRKYIDKSQYPSGFKTTIIKQAGVRIKSV |
| | | KSEQDEINLANWIISKYDPTYIKRDFNPSAKCQIIATSRSVADFDIVKMSN |
| | | KVQEIFFASSHLDKNVFDIGKSKSDHDSWFERNNVDRGIYTYSNVQGMN |
| | | LIFSNTKNTYLGVAVKAQNKFSSKMKRIQDINNFRITNHQSPLPIPDEIKIY |
| | | DDAGFLLNPPGVNPNIFGYQSCLLKPLENKEIISKTSFPEYSRLPADMIEV |
| | | NYKISNRLKFSNDQKGFIQFKDKLNLFKINSQELFSKRRRLSGQPILLVAS |
| | | FGDDWVVLDGRGLLRQVYYRGIAKPGSITISELLGFFTGDPIVDPIRGVVS |
| | | LGFKPGVLSQETLKTTSARIFAEKLPNLVLNNNVGLMSIDLGQTNPVSYR |
| | | LSEITSNMSVEHICSDFLSQDQISSIEKAKTSLDNLEEEIAIKAVDHLSDED |
| | | KINFANFSKLNLPEDTRQSLFEKYPELIGSKLDFGSMGSGTSYIADELIKFE |
| | | NKDAFYPSGKKKFDLSFSRDLRKKLSDETRKSYNDALFLEKRTNDKYLK |
| | | NAKRRKQIVRTVANSLVSKIEELGLTPVINIENLAMSGGFFDGRGKREKG |
| | | WDNFFKVKKENRWVMKDFHKAFSELSPHHGVIVIESPPYCTSVTCTKCN |
| | | FCDKKNRNGHKFTCQRCGLDANADLDIATENLEKVAISGKRMPGSERSS |
| | | DERKVAVARKAKSPKGKAIKGVKCTITDEPALLSANSQDCSQSTS |
| CasΦ.37 | 85 | MALSLAEVRERHFKGLRFRSSYLKRAGKILKKEGEAACVAYLTGKDEES |
| | | PPNFKPPAKCDVVAQSRPFEEWPIVQASVAVQSYVYGLTKEAFEAFNPG |
| | | TTKQSHEACLAATGIDTCGYSNVQGLNLIFRQAKNRYEGVITKVENRNK |
| | | KAKKKLTRKNEWRQKNGHSELPEAPEELTFNDEGRLLQPPGINPSLYTY |
| | | QQISPTPWSPKDSSILPPQYAGYERDPNAPIPFGVAKDRLTIASGCPGYIPE |
| | | WMRTAGEKTNPRTQKKFMHPGLSTRKNKRMRLPRSVRSAPLGALLVTI |
| | | HLGEDWLVLDVRGLLRNARWRGVAPKDISTQGLLNLFTGDPVIDTRRG |
| | | VVTFTYKPETVGIHSRTWLYKGKQTKEVLEKLTQDQTVALVAIDLGQTN |
| | | PVSAAASRVSRSGENLSIETVDRFFLPDELIKELRLYRMAHDRLEERIREE |
| | | STLALTEAQQAEVRALEHVVRDDAKNKVCAAFNLDAASLPWDQMTSN |
| | | TTYLSEAILAQGVSRDQVFFTPNPKKGSKEPVEVMRKDRAWVYAFKAK |
| | | LSEETRKAKNEALWALKRASPDYARLSKRREELCRRSVNMVINRAKKR |
| | | TQCQVVIPVLEDLNIGFFHGSGKRLPGWDNFFVAKKENRWLMNGLHKS |
| | | FSDLAVHRGFYVFEVMPHRTSITCPACGHCDSENRDGEAFVCLSCKRTY |
| | | HADLDVATHNLTQVAGTGLPMPEREHPGGTKKPGGSRKPESPQTHAPIL |
| | | HRTDYSESADRLGS |
| CasΦ.45 | 86 | QAVIKYLSDKGAVDPPDFRPPAKCNIIAQSRPFDEWPICKASMAIQQHIY |
| | | GLTKNEFDESSPGTSSASHEQWFAKTGVDTHGFTHVQGLNLIFQHAKKR |
| | | YEGVIKKVENYNEKERKKFEGINERRSKEGMPLLEPRLRTAFGDDGKFA |
| | | EKPGVNPSIYLYQQTSPRPYDKTKHPYVHAPFELKEITTIPTQDDRLKIPF |
| | | GAPGHVPEKHRSQLSMAKHKRRRAWYALSQNKPRPPKDGSKGRRSVR |
| | | DLADLKAASLADAIPLVSRVGFDWVVIDGRGLLRNLRWRKLAHEGMTV |
| | | EEMLGFFSGDPVIDPRRNVATFIYKAEHATVKSRKPIGGAKRAREELLKA |
| | | TASSDGVIRQVGLISVDLGQTNPVAYEISRMHQANGELVAEHLEYGLLN |
| | | DEQVNSIQRYRAAWDSMNESFRQKAIESLSMEAQDEIMQASTGAAKRT |
| | | REAVLTMFGPNATLPWSRMSSNTTCISDALIEVGKEEETNFVTSNGPRKR |
| | | TDAQWAAYLRPRVNPETRALLNQAVWDLMKRSDEYERLSKRKLEMAR |
| | | QCVNFVVARAEKLTQCNNIGIVLENLVVRNFHGSGRRESGWEGFFEPKR |
| | | ENRWFMQVLHKAFSDLAQHRGVMVFEVHPAYSSQTCPACRYVDPKNR |
| | | SSEDRERFKCLKCGRSFNADREVATFNIREIARTGVGLPKPDCERSRDVQ |
| | | TPGTARKSGRSLKSQDNLSEPKRVLQSKTRKKITSTETQNEPLATDLKT |
| CasΦ.38 | 87 | MIKEQSELSKLIEKYYPGKKFYSNDLKQAGKHLKKSEHLTAKESEELTV |
| | | EFLKSCKEKLYDFRPPAKALIISTSRPFEEWPIYKASESIQKYIYSLTKEEL |
| | | EKYNISTDKTSQENFFKESLIDNYGFANVSGLNLIFQHTKAIYDGVLKKV |
| | | NNRNNKILKKYKRKIEEGIEIDSPELEKAIDESGHFINPPGINKNIYCYQQV |
| | | SPTIFNSFKETKIICPFNYKRNPNDIIQKGVIDRLAIPFGEPGYIPDHQRDKV |
| | | NKHKKRIRKYYKNNENKNKDAILAKINIGEDWVLFDLRGLLRNAYWRK |
| | | LIPKQGITPQQLLDMFSGDPVIDPIKNNITFIYKESIIPIHSESIIKTKKSKELL |
| | | EKLTKDEQIALVSIDLGQTNPVAARFSRLSSDLKPEHVSSSFLPDELKNEI |
| | | CRYREKSDLLEIEIKNKAIKMLSQEQQDEIKLVNDISSEELKNSVCKKYNI |
| | | DNSKIPWDKMNGFTTFIADEFINNGGDKSLVYFTAKDKKSKKEKLVKLS |
| | | DKKIANSFKPKISKETREILNKITWDEKISSNEYKKLSKRKLEFARRATNY |
| | | LINQAKKATRLNNVVLVVEDLNSKFFHGSGKREDGWDNFFIPKKENRW |
| | | FIQALHKSLTDVSIHRGINVIEVRPERTSITCPKCGCCDKENRKGEDFKCI |
| | | KCDSVYHADLEVATFNIEKVAITGESMPKPDCERLGGEESIG |
| CasΦ.39 | 88 | VAFLDGKEVDEPYTLQPPAKCHILAVSRPIEEWPIARVTMAVQEHVYAL |
| | | PVHEVEKSRPETTEGSRSAWFKNSGVSNHGVTHAQTLNAILKNAYNVY |
| | | NGVIKKVENRNAKKRDSLAAKNKSRERKGLPHFKADPPELATDEQGYL |
| | | LQPPSPNSSVYLVQQHLRTPQIDLPSGYTGPVVDPRSPIPSLIPIDRLAIPPG |
| | | QPGYVPLHDREKLTSNKHRRMKLPKSLRAQGALPVCFRVFDDWAVVD |
| | | GRGLLRHAQYRRLAPKNVSIAELLELYTGDPVIDIKRNLMTFRFAEAVVE |
| | | VTARKIVEKYHNKYLLKLTEPKGKPVREIGLVSIDLNVQRLIALAIYRVH |
| | | QTGESQLALSPCLHREILPAKGLGDFDKYKSKFNQLTEEILTAAVQTLTS |
| | | AQQEEYQRYVEESSHEAKADLCLKYSITPHELAWDKMTSSTQYISRWLR |
| | | DHGWNASDFTQITKGRKKVERLWSDSRWAQELKPKLSNETRRKLEDAK |
| | | HDLQRANPEWQRLAKRKQEYSRHLANTVLSMAREYTACETVVIAIENL |
| | | PMKGGFVDGNGSRESGWDNFFTHKKENRWMIKDIHKALSDLAPNRGV |
| | | HVLEVNPQYTSQTCPECGHRDKANRDPIQRERFCCTHCGAQRHADLEV |
| | | ATHNIAMVATTGKSLTGKSLAPQRLQ |
| CasΦ.42 | 89 | LEIPEGEPGHVPWFQRMDIPEGQIGHVNKIQRFNFVHGKNSGKVKFSDKT |
| | | GRVKRYHHSKYKDATKPYKFLEESKKVSALDSILAHITIGDDWVVFDIRG |
| | | LYRNVFYRELAQKGLTAVQLLDLFTGDPVIDPKKGIITFSYKEGVVPVFS |
| | | QKIVSRFKSRDTLEKLTSQGPVALLSVDLGQNEPVAARVCSLKNINDKIA |
| | | LDNSCRIPFLDDYKKQIKDYRDSLDELEIKIRLEAINSLDVNQQVEIRDLD |
| | | VFSADRAKASTVDMFDIDPNLISWDSMSDARFSTQISDLYLKNGGDESR |
| | | VYFEINNKRIKRSDYNISQLVRPKLSDSTRKNLNDSIWKLKRTSEEYLKLS |
| | | KRKLELSRAVVNYTIRQSKLLSGINDIVIILEDLDVKKKFNGRGIRDIGWD |
| | | NFFSSRKENRWFIPAFHKSFSELSSNRGLCVIEVNPAWTSATCPDCGFCSK |
| | | ENRDGINFTCRKCGVSYHADIDVATLNIARVAVLGKPMSGPADRERLGG |
| | | TKKPRVARSRKDMKRKDISNGTVEVMVTA |
| CasΦ.46 | 90 | IPSFGYLDRLKIAKGQPGYIPEWQRETINPSKKVRRYWATNHEKIRNAIPL |
| | | VVFIGDDWVIIDGRGLLRDARRRKLADKNTTIEQLLEMVSNDPVIDSTRG |
| | | IATLSYVEGVVPVRSFIPIGEKKGREYLEKSTQKESVTLLSVDIGQINPVSC |
| | | GVYKVSNGCSKIDFLDKFFLDKKHLDAIQKYRTLQDSLEASIVNEALDEI |
| | | DPSFKKEYQNINSQTSNDVKKSLCTEYNIDPEAISWQDITAHSTLISDYLI |
| | | DNNITNDVYRTVNKAKYKTNDFGWYKKFSAKLSKEAREALNEKIWELK |
| | | IASSKYKKLSVRKKEIARTIANDCVKRAETYGDNVVVAMESLTKNNKV |
| | | MSGRGKRDPGWHNLGQAKVENRWFIQAISSAFEDKATHHGTPVLKVNP |
| | | AYTSQTCPSCGHCSKDNRSSKDRTIFVCKSCGEKFNADLDVATYNIAHV |
| | | AFSGKKLSPPSEKSSATKKPRSARKSKKSRKS |
| CasΦ.47 | 91 | SPIEKLLNGLLVKITFGNDWIICDARGLLDNVQKGIIHKSYFTNKSSLVDL |
| | | IDLFTCNPIVNYKNNVVTFCYKEGVVDVKSFTPIKSGPKTQENLIKKLKY |
| | | SRFQNEKDACVLGVGVDVGVTNPFAINGFKMPVDESSEWVMLNEPLFTI |
| | | ETSQAFREEIMAYQQRTDEMNDQFNQQSIDLLPPEYKVEFDNLPEDINEV |
| | | AKYNLLHTLNIPNNFLWDKMSNTTQFISDYLIQIGRGTETEKTITTKKGK |
| | | EKILTIRDVNWFNTFKPKISEETGKARTEIKRDLQKNSDQFQKLAKSREQ |
| | | SCRTWVNNVTEEAKIKSGCPLIIFVIEALVKDNRVFSGKGHRAIGWHNFG |
| | | KQKNERRWWVQAIHKAFQEQGVNHGYPVILCPPQYTSQTCPKCNHVDR |
| | | DNRSGEKFKCLKYGWIGNADLDVGAYNIARVAITGKALSKPLEQKKIKK |
| | | AKNKT |
| CasΦ.48 | 92 | LLDNVQKGIIHKSYFTNKSSLVDLIDLFTCNPIVNYKNNVVTFCYKEGVV |
| | | DVKSFTPIKSGPKTQENLIKKLKYSRFQNEKDACVLGVGVDVGVTNPFAI |
| | | NGFKMPVDESSEWVMLNEPLFTIETSQAFREEIMAYQQRTDEMNDQFN |
| | | QQSIDLLPPEYKVEFDNLPEDINEVAKYNLLHTLNIPNNFLWDKMSNTTQ |
| | | FISDYLIQIGRGTETEKTITTKKGKEKILTIRDVNWFNTFKPKISEETGKAR |
| | | TEIKRDLQKNSDQFQKLAKSREQSCRTWVNNVTEEAKIKSGCPLIIFVIEA |
| | | LVKDNRVFSGKGHRAIGWHNFGKQKNERRWWVQAIHKAFQEQGVNH |
| | | GYPVILCPPQYTSQTCPKCNHVDRDNRSGEKFKCLKYGWIGNADLDVG |
| | | AYNIARVAITGKALSKPLEQKKIKKAKNKT |
| CasΦ.49 | 93 | MIKPTVSQFLTPGFKLIRNHSRTAGLKLKNEGEEACKKFVRENEIPKDEC |
| | | PNFQGGPAIANIIAKSREFTEWEIYQSSLAIQEVIFTLPKDKLPEPILKEEW |
| | | RAQWLSEHGLDTVPYKEAAGLNLIIKNAVNTYKGVQVKVDNKNKNNL |
| | | AKINRKNEIAKLNGEQEISFEEIKAFDDKGYLLQKPSPNKSIYCYQSVSPK |
| | | PFITSKYHNVNLPEEYIGYYRKSNEPIVSPYQFDRLRIPIGEPGYVPKWQY |
| | | TFLSKKENKRRKLSKRIKNVSPILGIICIKKDWCVFDMRGLLRTNHWKKY |
| | | HKPTDSINDLFDYFTGDPVIDTKANVVRFRYKMENGIVNYKPVREKKGK |
| | | ELLENICDQNGSCKLATVDVGQNNPVAIGLFELKKVNGELTKTLISRHPT |
| | | PIDFCNKITAYRERYDKLESSIKLDAIKQLTSEQKIEVDNYNNNFTPQNTK |
| | | QIVCSKLNINPNDLPWDKMISGTHFISEKAQVSNKSEIYFTSTDKGKTKD |
| | | VMKSDYKWFQDYKPKLSKEVRDALSDIEWRLRRESLEFNKLSKSREQD |
| | | ARQLANWISSMCDVIGIENLVKKNNFFGGSGKREPGWDNFYKPKKENR |
| | | WWINAIHKALTELSQNKGKRVILLPAMRTSITCPKCKYCDSKNRNGEKF |
| | | NCLKCGIELNADIDVATENLATVAITAQSMPKPTCERSGDAKKPVRARK |
| | | AKAPEFHDKLAPSYTVVLREAVKRPAATKKAGQAKKKKEF |
| | | (Underlined sequence is Nuclear Localization Signal; SEQ ID NO: 1584) |
| CasΦ.12 | 94 | SNAPKKKRKVGIHGVPAAMIKPTVSQFLTPGFKLIRNHSRTAGLKLKNE |
| with NLS | | GEEACKKFVRENEIPKDECPNFQGGPAIANIIAKSREFTEWEIYQSSLAIQE |
| Signals | | VIFTLPKDKLPEPILKEEWRAQWLSEHGLDTVPYKEAAGLNLIIKNAVNT |
| | | YKGVQVKVDNKNKNNLAKINRKNEIAKLNGEQEISFEEIKAFDDKGYLL |
| | | QKPSPNKSIYCYQSVSPKPFITSKYHNVNLPEEYIGYYRKSNEPIVSPYQF |
| | | DRLRIPIGEPGYVPKWQYTFLSKKENKRRKLSKRIKNVSPILGIICIKKDW |
| | | CVFDMRGLLRTNHWKKYHKPTDSINDLFDYFTGDPVIDTKANVVRFRY |
| | | KMENGIVNYKPVREKKGKELLENICDQNGSCKLATVDVGQNNPVAIGL |
| | | FELKKVNGELTKTLISRHPTPIDFCNKITAYRERYDKLESSIKLDAIKQLTS |
| | | EQKIEVDNYNNNFTPQNTKQIVCSKLNINPNDLPWDKMISGTHFISEKAQ |
| | | VSNKSEIYFTSTDKGKTKDVMKSDYKWFQDYKPKLSKEVRDALSDIEW |
| | | RLRRESLEFNKLSKSREQDARQLANWISSMCDVIGIENLVKKNNFFGGSG |
| | | KREPGWDNFYKPKKENRWWINAIHKALTELSQNKGKRVILLPAMRTSIT |
| | | CPKCKYCDSKNRNGEKFNCLKCGIELNADIDVATENLATVAITAQSMPK |
| | | PTCERSGDAKKPVRARKAKAPEFHDKLAPSYTVVLREAVKRPAATKKA |
| | | GQAKKKKEF |
| | | (Underlined sequences Nuclear Localization Signals; SEQ ID NO: 1584) |
| CasM. | 95 | MIKNPSNRHSLPKVIISEVDHEKILEFKIKYEKLARLDRFEVKAMHYEGK |
| 1584 | | EIVFDEVLVNGGLIEVEYQDDNKTLFVKVGEKSYSIRGKKVGGKQRLLE |
| | | DRVSKTKVQLELSDGVVDNKGNLRKSRTERELIVADNIKLYSQIVGREV |
| | | TTTKEIYLVKRFLAYRSDLLFYYSFVDNFFKVAGNEKELWKINFDDATS |
| | | AQFMGYIPFMVNDNLKNDNAYLKDYVRNDVQIKDDLKKVQTIFSALRH |
| | | TLLHFNYEFFEKLFNGEDVGFDFDIGFLNLLIENIDKLNIDAKKEFIDNEKI |
| | | RLFGENLSLAKVYRLYSDICVNRVGFNKFINSMLIKDGVENQVLKAEFN |
| | | RKFGGNAYTIDIHSNQEYKRIYNEHKKLVIKVSTLKDGQAIRRGNKKISE |
| | | LKEQMKSMTKKNSLARLECKMRLAFGFLYGEYNNYKAFKNNFDTNIKN |
| | | SQFDVNDVEKSKAYFLSTYERRKPRTREKLEKVAKDIESLELKTVIANDT |
| | | LLKFILLMFVFMPQELKGDFLGFVKKYYHDVHSIDDDTKEQEEDVVEA |
| | | MSTSLKLKILGRNIRSLTLFKYALSSQVNYNSTDNIFYVEGNRYGKIYKK |
| | | LGISHNQEEFDKTLVVPLLRYYSSLFKLMNDFEIYSLAKANPTAVSLQEL |
| | | VDDETSPYKQGNYFNFNKMLRDIYGLTSDEIKSGQVVFMRNKIAHFDTE |
| | | VLLSKPLLGQTKMNLQRKDIVSFIEARGDIKELLGYDAINDFRMKVIHLR |
| | | TKMRVYSDKLQTMMDLLRNAKTPNDFYNVYKVKGVESINKHLLEVLA |
| | | QTAEERTVEKQIRDGNEKYDL |
| CasM. | 96 | MLKHKRKNKNSLARVVLSNYDSNNIYEIKIKYEKLAKLDKINIIEMDYD |
| 1730 | | ADNNVMFKKVLFNNKEIDLSHKDKTKINIELDNKKYNISAKKQIGKTHL |
| | | VVRDKQTSKISRIKKIQDTYYRGKDVFILDNNIEILDKKQTKDKFIVTLND |
| | | ITNDKTTSTEAELIDDTKDIFKKISAKKDLKSSDIYKIKRFISIRSNFSFYYT |
| | | FVDNYFKIFHAKKDKNKEELYKIKFKDEINIKPYLENILDNMKNKNGILY |
| | | DYADDREKVLNDLKNIQYVFTEFRHKLAHFDYNFLDNFFSNSVTDQYK |
| | | QKVNEIKLLDILLDNIDSLNVVPKQNYIEDETISVFDAKDIKLKRLYTYYI |
| | | KLTINYPGFKKLINSFFIQDGIENQELKEYINNKEKDTQVLKELDNKAYY |
| | | MDISQYRKYKNIYNKHKELVSEKELSSDGQKINSLNQKINKLKIEMKNIT |
| | | KPNALNRLIYRLRVAFGFIYKEYATINNFNKSFLQDTKIKRFENISQQDIK |
| | | NYLDISYQDKGKFFVKSKKTFKNKTTIKYTFEDLDLTLNEIITQDDIFVKV |
| | | IFLFSIFMPKELNGDFFGFINMYYHKMKNISYDTKDIDMLDTISQNMKLKI |
| | | LEQNIKKTYVFKYYLDLDSSIYSKLVQNIKITEDIDSKKYLYAKIFKYYQH |
| | | LYKLISDVEIYLLYKYNSKENLSITIDKDELKHRGYYNFQSLLIKNNINKD |
| | | DAYWSIVNMRNNLSHQNIDELVGHFCKGCLRKSTTDIAELWLRKDILTIT |
| | | NEIINKIESFKDIKITLGYDCVNDFTQKVKQYKQKLKASNERLAKKIEEK |
| | | QNQVVDEKNKEELEKKILNMKNIQKINRYILDIL |
| CasM. | 97 | MSQLKNPSNKNSLPRIIISDFNEIKINEIKIKYHKLDRLDKIIVKEMEIINNKI |
| 1816 | | FFKKILFNNQIKDINSENIELENYILAGEVKPSNTKIILNRDGKEKSFIVYD |
| | | GFTFKYKPNDKRISETKTNAKYILTIKDKTRHRESSTQRDILKSSIIETYKQ |
| | | ISGFENITSKDIYTIKRYIDFKNEMMFYYTFIDDFFFPITGKNKQDKKNNF |
| | | YNYKIKENAKKFISLINYRINDDFKNKNGILYDYLSNKEEIIINDFIHIQTIL |
| | | KDVRHAIAHFNFDFIQKLFDNEQAFNSKFDGIEILNILFNQKQEKYFEAQT |
| | | NYIEEETIKILDEKELSFKKLHSFYSQICQKKPAFNKLINSFIIQDGIENKEL |
| | | KDYISQKYNSKFDYYLDIHTCKIYKDIYNQHKKFVADKQFLENQKTDGQ |
| | | KIKKLNDQINQLKTKMNNLTKKNSLKRLEIKFRLAFGFIFTEYQTFKNFN |
| | | ERFIEDIKANKYSTKIELLDYGKIKEYISITHEEKRFFNYKTFNKKTNKNIN |
| | | KTIFQSLEKETFENLVKNDNLIKMMFLFQLLLPRELKGEFLGFILKIYHDL |
| | | KNIDNDTKPDEKSLSELNISTALKLKILVKNIRQINLFNYTISNNTKYEEKE |
| | | KRFYEEGNQWKDIYKKLYISHDFDIFDIHLIIPIIKYNINLYKLIGDFEVYL |
| | | LLKYLERNTNYKTLDKLIEAEELKYKGYYNFTTLLSKAINIALNDKEYH |
| | | NITHLRNNTSHQDIQNIISSFKNNKLLEQRENIIELISKESLKKKLHFDPIND |
| | | FTMKTLQLLKSLEVHSDKSEKIENLLKKEPLLPNDVYLLYKLKGIEFIKK |
| | | ELISNIGITKYEEKIQEKIAKGVEK |
| CasM. | 98 | ELCKIDFTDARSSSLIEYFKFAINDNLKNDRGYLKAYVNDVEQIRADLKK |
| 1862939 | | VGGKQRNLEDRVSRTKVQLTLTNHIEDREGKQRVSRTERELIVPQNIKLY |
| | | SQIVGREVKTTKEIYLIKRFLEYRSDLLFYYGFVDNFFKVEGNKKELCKI |
| | | DFTDARSSSLIEYFKFAINDNLKNDRGYLKAYVNDVEQIRTDLQKVKTIF |
| | | SKLRHALMHFDYDFFEKLFNGEEVGFDFDIKFLNIMIDKVEKLNIETKKE |
| | | FIEDEVITLFGERLSLKKLYGLFSHIAINRVAFNKFINSFLIKDGIENRALK |
| | | DFFNDEKGSQAYEIDIHSNAEYKALYVQHKKLVMATSAMSDGNEIAKK |
| | | NQEISELKEKMNAITKANSLARLEYKLRLAFGFIYTEYGDYTAFKNSFDR |
| | | DVKSAKYKELSVERLKAYYLATFKASKPQSHEKLEEVAKKIDRLSLKQL |
| | | IENETLLKFVLLLFTFMPQELKGEFLGFIKKYYHDKKHIEQDTKEKEEER |
| | | EGLSTGLKLKVLEKNIRSLSILKHALSFQVKYNKKDKNFYEEGNLHGKF |
| | | YKKLAISHNQEEFNKSVYAPLFRYYVALYKLINDFEIYSLAQHIVNNETL |
| | | ADQVGKAQFRQRGYFNFRKLVNCTYATAQNSSYNVLIFMRNDISHLSYE |
| | | PLFNCPLEEKASYKQKIRGREKIISVKPLSESRAEIVRFIASQTDMKKLLG |
| | | YDAVNDFNMKMVQLRRRLSVYANKQETIEKMINKAKTPNDFYNLYKL |
| | | KGIECINQHLLKVIGVTEAEKRIEKQIEEGNEKY |
| CasM. | 99 | MLKKPSNRYALPKVILSTVDHEKILEFKVKYEKLARLDRLVVERMHFDG |
| 1862895 | | ESVVFDEVIANSGDLEIAYQDDHRKLLIQAAGKSYTITGKKVGGKKRKL |
| | | EERISRAKIQLTLTDGQEDQHRRIRATVTEKALLEPKEDRDIYSKISDRKI |
| | | KTSKEIYLVKRFLSYRSDLLFYYFFVDNFFKVGNNKQELWKIKFQNQPEL |
| | | IEYFRFIINDRFKNAKNDKFDNYLKNDKAIQEDLEKIQKVFEKLRHALMH |
| | | YDYGFFEKLFGGEDQGFDLDIAFLDNFVKKIDKLNIDTKKEFVDDEKIKI |
| | | FGEDLNLADLYKLYASISINRVGFNRVVNEMIIKDGIEKSELKRAFEKKL |
| | | DKTYALDIHSDPSYKKLYNEHKRLVTEVSTYTDGNKIKEGNQKIAKLKY |
| | | EMKEITKKNALVRLECKMRLAFGLIYGRYDTHEAFKNGFDTDLKRGEF |
| | | AQIGSEEAIGYFNTTFEKSKPKSKEEIKKIARQIDNLSLSTLIEDDPLMKFI |
| | | VLMFLFVPRELKGEFLGFWRKYYHDIHSIDSDAKSDEMPDEVSLSLKLKI |
| | | LTRNIRRLNLFEYSLSEKIKYSPKNTQFYTDKSPYQKVYKRLKISHNKEEF |
| | | DKTLLVPLFRYYSILFKLINDFEIYSLAKANPDASSLSELTKTKHGFRGHY |
| | | NFTTLMMDAHKVSQGDSKKHFGIRGEIAHINTKDLIYDPLFRKSKMAQQ |
| | | RNDVIDFVLKYEKEIKAVLGYDAINDFRMKVVQLRTKLKVYSDKTQTIE |
| | | KLLNEVEAPDDFYVLYKVKGVEAINKYLLEIVSVTQAEEEIERKIITGNK |
| | | RYNT |
| CasM. | 100 | MLKHKRKNKNSLARVVLSNYDSNNIYEIKIKYEKLAKLDKINIIEMDYD |
| 1862903 | | ADNNVMFKKVLFNNKEIDLSHKDKTKINIELDNKKYNISAKKQIGKTHL |
| | | VVRNKQTSKISRIKKIQDTYYRGKDVFILDNNIEILDKKQTKDKFIVTLND |
| | | ITNNKTTSTEAELIDDTKDIFKKISAKKDLKSSDIYKIKRFISIRSNFSFYYT |
| | | FVDNYFKIFHAKKDKNKEELYKIKFKDEINIKPYLENILDNMKNKNGILY |
| | | NYANDRKKVLNDLRNIQYVFKEFRHKLAHFDYNFLDNFFSNSVEEKYK |
| | | QKVNEIKLLDILLDNIDSLNVVPKQNYIEDETISVFDAKDIKLKRLYTYYI |
| | | KLTINYPGFKKLINSFFIQDGIENQELKEYINNKEKDTQVLKELDNKAYY |
| | | MDISQYRKYKNIYNKHKELVSEKELSSDGKKINSLNQKINKLKIDMKNIT |
| | | KPNALNRLIYRLRVAFGFIYKEYATINNFNKSFLQDTKTKRFENISQQDIK |
| | | SYLDISYQDKGKFFVKSKKTFKNKTTVKYTFEDLDLTLNEIITQDDIFVK |
| | | VIFLFSIFMPKELNGDFFGFINMYYHKMKNISYDTKDIDMLDTISQNMKL |
| | | KILEQNIKKTYVFKYYLDLDSSIYSKLVQNIKITEDIDSKKYLYAKIFKYY |
| | | QHLYKLISDVEIYLLYKYNSKENLSITIDKDELKHRGYYNFQSLLIKNNIN |
| | | KDDAYWSIVNMRNNLSHQNIDELVGHFCKGCLRKSTTDIAELWLRKDIL |
| | | TITNEIINKIESFKDIKITLGYDCVNDFTQKVKQYKQKLKASNERLAKKIE |
| | | EKQNQVVDEKNKEELEKNILNMKNIQKINRYILDIL |
| CasM. | 101 | MIKKPSNRHALPKVIISKVDNQNILEFKIKYKKLSRLDRVEIKTMHYDDR |
| 1862909 | | AIVFDEVIINGGLIDVEYRDNHKTIFVKVGDKSYSISGQKVGGKERLLEN |
| | | RISQTKVQLELKDEATNRVSKTERELIVDDNIKLYSQIVGRDVKTTKDIY |
| | | LIKRFLGYRSDLLFYYGFVNNFFHVANNRPEFWKIDFNDNRNSKLIEYFIF |
| | | TINDHLKNDENYLKDYISDRGQIVDDLENIKHIFSALRHGLMHFDYDFFE |
| | | ALFNGEDIDIKMDNQGNTQPLSSLNIKFLDIMIDKLDKLNIDTKKEFIDAE |
| | | KITIFGEELSLAKLYRFYAHTAINRVAFNKLINSFIIENGVENQSLKEYFNQ |
| | | QAGGIAYEIDIHQNREYKNLYNEHKKLVSRVLSISDGQEIATLNQKIVEL |
| | | KEQMKQITKINSIKRLEYKLRLAFGFIYTEYKNYEEFKNSFDTDIKNGRFT |
| | | PKDEDGNKRAFDSRELEHLKGYYKATLQTQKPQTDEKMEEVSKRVDRL |
| | | SLKSLIGDDTLLKFILLMFTFMPQELKGEFLGFIKKYYHDTKHIDQDTISD |
| | | SDDTIEEGLSIGLKLKILDKNIRSLSILKHSLSFQTKYNKKDRSYYEDGNIH |
| | | GKFFKKLGISHNQEEFNKSVYAPLFRYYSALYKLINDFEIYTLSLHIVGNE |
| | | TLSDQVNKPQFLSGRYFNFRKLLTQSYNISNNSTHSVIFNAVINMRNDISH |
| | | LSYEPLLDCPLNGKKSYKRKIRNQFRTINIKPLVESRKMIIDFITLQTDMQ |
| | | KVLGCDAVNDFTMKIVQLRTRLKAYANKEQTIEKMITEAKTPNDFYNIY |
| | | KVKGVEAINKYLLEVIGETQVEKEIREEIERGNIANS |
| CasM. | 102 | MVKNPANRHALPKVIISEVDNNNILEFKIKYEKLARLDKVEVKSMHFDN |
| 1862917 | | NKQVVFDEVVINGGLIEPTYEDKHKKLVVTAGEKSYSIVGQKVGGKPRL |
| | | LEDRVSKTKVQLELTNYVEDKEGKKRVSKTERELIVADNIELYSQIVGRE |
| | | VKTTKEIYLIKRFLEYRSDLLFYYGFVDNFFKVAGNGKELWKIDFTNSDS |
| | | LHLIEYFKFSINDNLKNDENYLKNYVSDNTKIENDLVKCQNNFNSLRHA |
| | | LMHFDYDFFEKLFNGEDVGFDFDIEFLNIMIDKVDKLNIDTKKEFIDDEE |
| | | VTLFGEALSLKKLYGLFSHIAINRVAFNKLINSFIIEDGIENKELKDFFNNK |
| | | KESQAYEIDIHSNAEYKALYVQHKKLVMATSAMTDGDEIAKKNQEISDL |
| | | KEKMKVITKENSLARLEHKLRLAFGFIYTEYKDYKTFKKHFDQDIKGAK |
| | | YKGLNVEKLKEYYETTLKNSKPKTDEKLEDVAKKIDKLSLKELIDDDTL |
| | | LKFVLLLFIFMPQELKGDFLGFIKKYYHDKKHIDQDTKDKDTEIEELSTG |
| | | LKLKVLDKNIRSLSILKHSFSFQVKYNRKDKNFYEDGNLHGKFYKKLSIS |
| | | HNQEEFNKSVYAPLFRYYSALYKLINDFEIYALAQHVENHETLADQVNK |
| | | SQFIQKSYFNFRKLLDNTDSISQSSSYNTLIVMRNDISHLSYEPLFNYPLDE |
| | | RKSYKKKTQKGVKTFHVELLYISRAKIIELISLQTDMKKLLGYDAVNDF |
| | | NMKVVHLRKRLSVYANKEESIRKMQADAKTPNDFYNIYKVKGVESINQ |
| | | HLLKVIGVTEAEKSIEKQINEGNKKHNT |
| CasM. | 103 | MIKNPSNRYALPKVIISKIDNQNILEFKIKYKKLSKLDIVKVKSMHYDDR |
| 1862921 | | AIIFDEVIVNDGLIDVEYRDNHKTIFVKVGNKSYSISGQKVGGKERLLEN |
| | | RVSKTKVQLELKDKATNRVSKTERELIVDDNIKIYSQIVGRDVKTTKDIY |
| | | LIKRFLAYRSDLLFYYGFVNNFFHVANNRSEFWKIDFNDSNNSKLIEYFK |
| | | FTINDHLKNDENYLKDYISDNEKLKNDLIKVKNSFEKIRHALMHFDYDFF |
| | | VKLFNGEDVGLELDIEFLDIMIDKLDKLNIDTKKEFIDDEKITIFGEELSLA |
| | | KLYRFYAHTAINRVAFNKLINSFIIENGVENQSLKEYFNQQAGGIAYEIDI |
| | | HQNREYKNLYNEHKKLVSRVLSISDGQEIAILNQKIAKLKDQMKQITKA |
| | | NSIKRLEYKLRLALGFIYTEYENYEEFKNNFDTDIKNGRFTPKDNDGNKR |
| | | AFDSRELEQLKGYYEATIQTQKPKTDEKIEEVSKKIDRLSLKSLIADDILL |
| | | KFILLMFTFMPQELKGEFLGFIKKYYHDTKHIDQDTISDSDDTIETLSIGLK |
| | | LKILDKNIRSLSILKHSLSFQTKYNKKDRNYYEDGNIHGKFFKKLGISHN |
| | | QEEFNKSVYAPLFRYYSALYKLINDFEIYTLSLHIVGSETLTDQVNKSQFL |
| | | SGRYFNFRKLLTQSYHINNNSTHSTIFNAVINMRNDISHLSYEPLFDCPLN |
| | | GKKSYKRKIRNQFKTINIKPLVESRKIIIDFITLQTDMQKVLGYDAVNDFT |
| | | MKIVQLRTRLKAYANKEQTIQKMITEAKTPNDFYNIYKVQGVEEINKYL |
| | | LEVIGETQAEKEIREKIERGNIANF |
| CasM. | 104 | MTKKPSNRNSLPKVIINKVDESSILEFKIKYEKLARLDRFEVRSMRYDGD |
| 1862947 | | GRIIFDEVVANAGLLDVDYEDDNRTIVVKIENKAYNIYGKKVGGEKRLN |
| | | GKISKAKVQLILTDSIRKNANDTHRHSLTERELINKNEVDLYSKIAEREIS |
| | | TTKDIYLVKRFLAYRSDLLLYYAFINHYVRVNGNKKEFWKTEIDDKIIDY |
| | | FIYTINDTLKNKEGYLEKYIVDRDQIKKDLEKIKQIFSHLRHKLMHYDFR |
| | | FFTDLFDGKDVDIKVDNSIQKISELLDIEFLNIVIDKLEKLNIDAKKEFIDD |
| | | EKITLFGQEIELKKLYSLYAHTSINRVAFNKLINSFLIKDGVENKELKEYF |
| | | NAHNQGKESYYIDIHQNQEYKKLYIEHKNLVAKLSATTDGKEIAKINRE |
| | | LADKKEQMKQITKANSLKRLEYKLRLAFGFIYTEYKDYERFKNSFDTDT |
| | | KKKKFDAIDNAKIIEYFEATNKAKKIEKLEEILKGIDKLSLKTLIQDDILLK |
| | | FLLLFFTFLPQEIKGEFLGFIKKYYHDITSLDEDTKDKDDEITELPRSLKLK |
| | | IFSKNIRKLSILKHSLSYQIKYNKKESSYYEAGNVFNKMFKKQAISHNLEE |
| | | FGKSIYLPMLKYYSALYKLINDFEIYALYKDMDTSETLSQQVDKQEYKR |
| | | NEYFNFETLLRKKFGNDIEKVLVTYRNKIAHLDFNFLYDKPINKFISLYKS |
| | | REKIVNYIKNHDIQAVLKYDAVNDFVMKVIQLRTKLKVYADKEQTIESM |
| | | IQNTQNPNGFYNIYKVKAVENINRHLLKVIGYTESEKAVEEKIRAGNTSK |
| | | S |
| CasM. | 105 | MEKIKKPSNRNSIPSIIISDYDANKIKEIKVKYLKLARLDKITIQDMEIVDNI |
| 1422 | | VEFKKILLNGVEHTIIDNQKIEFDNYEITGCIKPSNKRRDGRISQAKYVVTI |
| | | TDKYLRENEKEKRFKSTERELPNNTLLSRYKQISGFDTLTSKDIYKIKRYI |
| | | DFKNEMLFYFQFIEEFFNPLLPKGKNFYDLNIEQNKDKVAKFIVYRLNDD |
| | | FKNKSLNSYITDTCMIINDFKKIQKILSDFRHALAHFDFDFIQKFFDDQLD |
| | | KNKFDINTISLIETLLDQKEEKNYQEKNNYIDDNDILTIFDEKGSKFSKLH |
| | | NFYTKISQKKPAFNKLINSFLSQDGVPNEEFKSYLVTKKLDFFEDIHSNKE |
| | | YKKIYIQHKNLVIKKQKEESQEKPDGQKLKNYNDELQKLKDEMNTITKQ |
| | | NSLNRLEVKLRLAFGFIANEYNYNFKNFNDEFTNDVKNEQKIKAFKNSS |
| | | NEKLKEYFESTFIEKRFFHFSVNFFNKKTKKEETKQKNIFNSIENETLEEL |
| | | VKESPLLQIITLLYLFIPRELQGEFVGFILKIYHHTKNITSDTKEDEISIEDA |
| | | QNSFSLKFKILAKNLRGLQLFHYSLSHNTLYNNKQCFFYEKGNRWQSVY |
| | | KSFQISHNQDEFDIHLVIPVIKYYINLNKLMGDFEIYALLKYADKNSITVK |
| | | LSDITSRDDLKYNGHYNFATLLFKTFGIDTNYKQNKVSIQNIKKTRNNLA |
| | | HQNIENMLKAFENSEIFAQREEIVNYLQTEHRMQEVLHYNPINDFTMKT |
| | | VQYLKSLSVHSQKEGKIADIHKKESLVPNDYYLIYKLKAIELLKQKVIEVI |
| | | GESEDEKKIKNAIAKEEQIKKGNN |
| CasM. | 106 | MMTKKPANRHALPKVIISEVDNTNILEFKIKYEKLARLDRVEVKAMHYE |
| 1740 | | DGRIIFDEVVVNGGLIEVEYQDDHKTLFVQVGEKSYSISGQKVGGKQRL |
| | | LEDRVSKTKVQLELSDGSSERVSRTERELIVADNIKLYSQIVGHEVKTTK |
| | | EIYLAKRFLGYRSDLLFYYGFVDNFFRESKNLKYGKQPVELWEDKFQVN |
| | | DKLTAYTKFMFNDDLQNSESYLKEYVKDNHKIKNDLESARDIFATFRHN |
| | | LMHFNYSFFTRLFNGEDVKIKNLQTKKFESLSDVLRNVEFLNKVIQSIDK |
| | | LNIDTRKEFIDKEKITLFNEELDLQQLYGFFAYTAINRVAFNKLINSFIIKD |
| | | GIENEQLKEYFNQRVDGTAYEIDIHQNREYKELYKKHKNLVSKVSTLSD |
| | | GKEIARGNTEISVLKEQMNKITKANSLKRLEHKLRLAFGFIYTEYGSYKA |
| | | FVSRFNEDTKRKKIKNVEFEKIGVEKQKEYYESTFTSNNKDKLGELIQEY |
| | | EKLSLNDLIENDTFLKVILLLFIFMPKEVKGDFLGFIKKYYHDTKHIEEDT |
| | | KEKDEGFTNTLPIGLKLKIVERNIAKLSVLKHSLSLKVKYNRGQYEEDNT |
| | | YRKVFKKLNISHNQEEFHKSMFSPLLRYYASLYKLINDFEIYTLSHYITDK |
| | | YSTLNKVIASEQFHYRYGWNREEKKGELVKTDNYTFSTLLSKKYGHKN |
| | | SQEISEMRNKISHFDEKILFKFPLEEVSSVPKGKGKYKKDEPIKSLKEKRE |
| | | EIVSLMEKQTDMQKVLGYDAINDFRMKTVQFQTKLKVYSNKEETIKKM |
| | | IVEAKTPNDYYNIYKVKGVEGINEHLLNVIGETEAEKSIQEQIAEGNKVN |
| | | V |
| Cas14a. | 107 | MAGKKKDKDVINKTLSVRIIRPRYSDDIEKEISDEKAKRKQDGKTGELDR |
| 280852 | | AFFSELKSRNPDIITNDELFPLFTEIQKNLTEIYNKSISLLYMKLIVEEEGGS |
| | | TASALSAGPYKECKARFNSYISLGLRQKIQSNFRRKELKGFQVSLPTAKS |
| | | DRFPIPFCHQVENGKGGFKVYETGDDFIFEVPLIKYTATNKKSTSGKNYT |
| | | KVQLNNPPVPMNVPLLLSTMRRRQTKKGMQWNKDEGTNAELRRVMSG |
| | | EYKVSYAEIIRRTRFGKHDDWFVNFSIKFKNKTDELNQNVRGGIDIGVSN |
| | | PLVCAVTNGLDRYIVANNDIMAFNERAMARRRTLLRKNRFKRSGHGAK |
| | | NKLEPITVLTEKNERFRKSILQRWAREVAEFFKRTSASVVNMEDLSGITE |
| | | REDFFSTKLRTTWNYRLMQTTIENKLKEYGIAVNYISPKYTSQTCHSCGK |
| | | RNDYFTFSYRSENNYPPFECKECNKVKCNADFNAAKNIALKVVL |
| | 108 | MRISKTLSLRIVRPFYTPEVEAGIKAEKDKREAQGQTRSLDAKFFNELKK |
| | | KHSEIILSSEFYSLLSEVQRQLTSIYNHAMSNLYHKIIVEGEKTSTSKALSN |
| | | IGYDECKAIFPSYMALGLRQKIQSNFRRRDLKNFRMAVPTAKSDKFPIPIY |
| | | RQVDGSKGGFKISENDGKDFIVELPLVDYVAEEVKTAKGRFTKINISKPP |
| | | KIKNIPVILSTLRRRQSGQWFSDDGTNAEIRRVISGEYKVSWIEIVRRTRF |
| | | GKHDDWFVNMVIKYDKPEEGLDSKVVGGIDVGVSSPLVCALNNSLDRY |
| | | FVKSSDIIAFNKRAMARRRTLLRQNKYKRSGHGSKNKLEPITVLTEKNER |
| | | FKKSIMQRWAKEVAEFFRGKGASVVRMEELSGLKEKDNFFSSYLRMYW |
| | | NYGQLQQIIENKLKEYGIKVNYVSPKDTSKKCHSCTHINEFFTFEYRQKN |
| | | NFPLFKCEKCGVECSADYNAAKNMAIA |
| Cas14 | 109 | MKDYIRKTLSLRILRPYYGEEIEKEIAAAKKKSQAEGGDGALDNKFWDR |
| ortholog 3 | | LKAEHPEIISSREFYDLLDAIQRETTLYYNRAISKLYHSLIVEREQVSTAK |
| | | ALSAGPYHEFREKFNAYISLGLREKIQSNFRRKELARYQVALPTAKSDTF |
| | | PIPIYKGFDKNGKGGFKVREIENGDFVIDLPLMAYHRVGGKAGREYIELD |
| | | RPPAVLNVPVILSTSRRRANKTWFRDEGTDAEIRRVMAGEYKVSWVEIL |
| | | QRKRFGKPYGGWYVNFTIKYQPRDYGLDPKVKGGIDIGLSSPLVCAVTN |
| | | SLARLTIRDNDLVAFNRKAMARRRTLLRQNRYKRSGHGSANKLKPIEAL |
| | | TEKNELYRKAIMRRWAREAADFFRQHRAATVNMEDLTGIKDREDYFSQ |
| | | MLRCYWNYSQLQTMLENKLKEYGIAVKYIEPKDTSKTCHSCGHVNEYF |
| | | DFNYRSAHKFPMFKCEKCGVECGADYNAARNIAQA |
| Cas14 | 110 | VKISKTLSLRIIRPYYTPEVESAIKAEKDKREAQGQTRNLDAKFFNELKK |
| ortholog 4 | | KHPQIILSGEFYSLLFEMQRQLTSIYNRAMSSLYHKIIVEGEKTSTSKALS |
| | | DIGYDECKSVFPSYIALGLRQKIQSNFRRKELKGFRMAVPTAKSDKFPIPI |
| | | YKQVDDGKGGFKISENKEGDFIVELPLVEYTAEDVKTAKGKFTKINISKP |
| | | PKIKNIPVILSTLRRKQSGQWFSDEGTNAEIRRVISGEYKVSWIEVVRRTR |
| | | FGKHDDWFLNIVIKYDKTEDGLDPEVVGGIDVGVSTPLVCAVNNSLDRY |
| | | FVKSSDIIAFKKRAMARRRTLLRQNRFKRSGHGSKSKLEPITILTEKNERF |
| | | KKSIMQRWAKEVAEFFKGERASVVQMEELSGLKEKDNFFGSYLRMYW |
| | | NYGQLQQIIENKLKEYGIKVNYVSPKDTSKKCHSCGYINEFFTFEFRQKN |
| | | NFPLFKCKKCGVECNADYNAAKNIAIA |
| Cas14 | 111 | VPITKTISLRILRPYYPPEIEAKIKAEKEKRKENGDTGSLNSSYYRELKKEY |
| ortholog 5 | | PSIIINDEFFPLLSEMQRNITSIYNRTISHLYHRLIIKKESISTAKALSEGPYR |
| | | DFKSTFNSYIALGLRQKVQSNFRKKDLMAFKIALPTAKSDKFPIPIYMQT |
| | | NFKIKESPDSDFIIELPLVEYIAKETKGKNKMFTKVEILSPPKVKNIPVILST |
| | | RRRKESGQWFSDEGTNAEIRRIISGEYKVSWIEIVKRTRFGKHDWFVNM |
| | | VISFEESQEGLDPDVIGGIDIGVSKPLICAINNSLDRYIVKGDDIIAFNRRAL |
| | | SRRRSLLRRNRLKRSGHGSRNKLEPITVLTEKNERFKKSIMQRWAKEVA |
| | | EFFKSKRASIVQMEELTGIKEREDFFSKTLRMYWNYGQLQKTVENKLRE |
| | | YGIEVRYASPKDTSRRCHSCGHINDYFTFEFRQQNNFPLFKCMNCGIECS |
| | | ADYNAARNIAIAR |
| Cas14 | 112 | MEVQKTVMKTLSLRILRPLYSQEIEKEIKEEKERRKQAGGTGELDGGFY |
| ortholog 6 | | KKLEKKHSEMFSFDRLNLLLNQLQREIAKVYNHAISELYIATIAQGNKSN |
| | | KHYISSIVYNRAYGYFYNAYIALGICSKVEANFRSNELLTQQSALPTAKS |
| | | DNFPIVLHKQKGAEGEDGGFRISTEGSDLIFEIPIPFYEYNGENRKEPYKW |
| | | VKKGGQKPVLKLILSTFRRQRNKGWAKDEGTDAEIRKVTEGKYQVSQIE |
| | | INRGKKLGEHQKWFANFSIEQPIYERKPNRSIVGGLDVGIRSPLVCAINNS |
| | | FSRYSVDSNDVFKFSKQVFAFRRRLLSKNSLKRKGHGAAHKLEPITEMT |
| | | EKNDKFRKKIIERWAKEVTNFFVKNQVGIVQIEDLSTMKDREDHFFNQY |
| | | LRGFWPYYQMQTLIENKLKEYGIEVKRVQAKYTSQLCSNPNCRYWNNY |
| | | FNFEYRKVNKFPKFKCEKCNLEISADYNAARNLSTPDIEKFVAKATKGIN |
| | | LPEK |
| Cas14 | 113 | MEEAKTVSKTLSLRILRPLYSAEIEKEIKEEKERRKQGGKSGELDSGFYK |
| ortholog 7 | | KLEKKHTQMFGWDKLNLMLSQLQRQIARVFNQSISELYIETVIQGKKSN |
| | | KHYTSKIVYNRAYSVFYNAYLALGITSKVEANFRSTELLMQKSSLPTAKS |
| | | DNFPILLHKQKGVEGEEGGFKISADGNDLIFEIPIPFYEYDSANKKEPFKW |
| | | IKKGGQKPTIKLILSTFRRQRNKGWAKDEGTDAEIRKVIEGKYQVSHIEIN |
| | | RGKKLGDHQKWFVNFTIEQPIYERKLDKNIIGGIDVGIKSPLVCAVNNSF |
| | | ARYSVDSNDVLKFSKQAFAFRRRLLSKNSLKRSGHGSKNKLDPITRMTE |
| | | KNDRFRKKIIERWAKEVTNFFIKNQVGTVQIEDLSTMKDRQDNFFNQYL |
| | | RGFWPYYQMQNLIENKLKEYGIETKRIKARYTSQLCSNPSCRHWNSYFS |
| | | FDHRKTNNFPKFKCEKCALEISADYNAARNISTPDIEKFVAKATKGINLP |
| | | DKNENVILE |
| Cas14a.1 | 114 | MAKNTITKTLKLRIVRPYNSAEVEKIVADEKNNREKIALEKNKDKVKEA |
| | | CSKHLKVAAYCTTQVERNACLFCKARKLDDKFYQKLRGQFPDAVFWQ |
| | | EISEIFRQLQKQAAEIYNQSLIELYYEIFIKGKGIANASSVEHYLSDVCYTR |
| | | AAELFKNAAIASGLRSKIKSNFRLKELKNMKSGLPTTKSDNFPIPLVKQK |
| | | GGQYTGFEISNHNSDFIIKIPFGRWQVKKEIDKYRPWEKFDFEQVQKSPK |
| | | PISLLLSTQRRKRNKGWSKDEGTEAEIKKVMNGDYQTSYIEVKRGSKIGE |
| | | KSAWMLNLSIDVPKIDKGVDPSIIGGIDVGVKSPLVCAINNAFSRYSISDN |
| | | DLFHFNKKMFARRRILLKKNRHKRAGHGAKNKLKPITILTEKSERFRKK |
| | | LIERWACEIADFFIKNKVGTVQMENLESMKRKEDSYFNIRLRGFWPYAE |
| | | MQNKIEFKLKQYGIEIRKVAPNNTSKTCSKCGHLNNYFNFEYRKKNKFP |
| | | HFKCEKCNFKENADYNAALNISNPKLKSTKEEP |
| Cas14 | 115 | MERQKVPQIRKIVRVVPLRILRPKYSDVIENALKKFKEKGDDTNTNDFW |
| ortholog 9 | | RAIRDRDTEFFRKELNFSEDEINQLERDTLFRVGLDNRVLFSYFDFLQEKL |
| | | MKDYNKIISKLFINRQSKSSFENDLTDEEVEELIEKDVTPFYGAYIGKGIK |
| | | SVIKSNLGGKFIKSVKIDRETKKVTKLTAINIGLMGLPVAKSDTFPIKIIKT |
| | | NPDYITFQKSTKENLQKIEDYETGIEYGDLLVQITIPWFKNENKDFSLIKT |
| | | KEAIEYYKLNGVGKKDLLNINLVLTTYHIRKKKSWQIDGSSQSLVREMA |
| | | NGELEEKWKSFFDTFIKKYGDEGKSALVKRRVNKKSRAKGEKGRELNL |
| | | DERIKRLYDSIKAKSFPSEINLIPENYKWKLHFSIEIPPMVNDIDSNLYGGI |
| | | DFGEQNIATLCVKNIEKDDYDFLTIYGNDLLKHAQASYARRRIMRVQDE |
| | | YKARGHGKSRKTKAQEDYSERMQKLRQKITERLVKQISDFFLWRNKFH |
| | | MAVCSLRYEDLNTLYKGESVKAKRMRQFINKQQLFNGIERKLKDYNSEI |
| | | YVNSRYPHYTSRLCSKCGKLNLYFDFLKFRTKNIIIRKNPDGSEIKYMPFF |
| | | ICEFCGWKQAGDKNASANIADKDYQDKLNKEKEFCNIRKPKSKKEDIGE |
| | | ENEEERDYSRRFNRNSFIYNSLKKDNKLNQEKLFDEWKNQLKRKIDGRN |
| | | KFEPKEYKDRFSYLFAYYQEIIKNESES |
| Cas14 | 116 | MVPTELITKTLQLRVIRPLYFEEIEKELAELKEQKEKEFEETNSLLLESKKI |
| ortholog 10 | | DAKSLKKLKRKARSSAAVEFWKIAKEKYPDILTKPEMEFIFSEMQKMMA |
| | | RFYNKSMTNIFIEMNNDEKVNPLSLISKASTEANQVIKCSSISSGLNRKIA |
| | | GSINKTKFKQVRDGLISLPTARTETFPISFYKSTANKDEIPISKINLPSEEEA |
| | | DLTITLPFPFFEIKKEKKGQKAYSYFNIIEKSGRSNNKIDLLLSTHRRQRRK |
| | | GWKEEGGTSAEIRRLMEGEFDKEWEIYLGEAEKSEKAKNDLIKNMTRG |
| | | KLSKDIKEQLEDIQVKYFSDNNVESWNDLSKEQKQELSKLRKKKVEELK |
| | | DWKHVKEILKTRAKIGWVELKRGKRQRDRNKWFVNITITRPPFINKELD |
| | | DTKFGGIDLGVKVPFVCAVHGSPARLIIKENEILQFNKMVSARNRQITKD |
| | | SEQRKGRGKKNKFIKKEIFNERNELFRKKIIERWANQIVKFFEDQKCATV |
| | | QIENLESFDRTSYK |
| Cas14 | 117 | MKSDTKDKKIIIHQTKTLSLRIVKPQSIPMEEFTDLVRYHQMIIFPVYNNG |
| ortholog 11 | | AIDLYKKLFKAKIQKGNEARAIKYFMNKIVYAPIANTVKNSYIALGYSTK |
| | | MQSSFSGKRLWDLRFGEATPPTIKADFPLPFYNQSGFKVSSENGEFIIGIPF |
| | | GQYTKKTVSDIEKKTSFAWDKFTLEDTTKKTLIELLLSTKTRKMNEGWK |
| | | NNEGTEAEIKRVMDGTYQVTSLEILQRDDSWFVNFNIAYDSLKKQPDRD |
| | | KIAGIHMGITRPLTAVIYNNKYRALSIYPNTVMHLTQKQLARIKEQRTNS |
| | | KYATGGHGRNAKVTGTDTLSEAYRQRRKKIIEDWIASIVKFAINNEIGTI |
| | | YLEDISNTNSFFAAREQKLIYLEDISNTNSFLSTYKYPISAISDTLQHKLEE |
| | | KAIQVIRKKAYYVNQICSLCGHYNKGFTYQFRRKNKFPKMKCQGCLEA |
| | | TSTEFNAAANVANPDYEKLLIKHGLLQLKK |
| Cas14 | 118 | MSTITRQVRLSPTPEQSRLLMAHCQQYISTVNVLVAAFDSEVLTGKVSTK |
| ortholog 12 | | DFRAALPSAVKNQALRDAQSVFKRSVELGCLPVLKKPHCQWNNQNWR |
| | | VEGDQLILPICKDGKTQQERFRCAAVALEGKAGILRIKKKRGKWIADLT |
| | | VTQEDAPESSGSAIMGVDLGIKVPAVAHIGGKGTRFFGNGRSQRSMRRR |
| | | FYARRKTLQKAKKLRAVRKSKGKEARWMKTINHQLSRQIVNHAHALG |
| | | VGTIKIEALQGIRKGTTRKSRGAAARKNNRMTNTWSFSQLTLFITYKAQ |
| | | RQGITVEQVDPAYTSQDCPACRARNGAQDRTYVCSECGWRGHRDTVG |
| | | AINISRRAGLSGHRRGATGA |
| Cas14 | 119 | MIAQKTIKIKLNPTKEQIIKLNSIIEEYIKVSNFTAKKIAEIQESFTDSGLTQ |
| ortholog 13 | | GTCSECGKEKTYRKYHLLKKDNKLFCITCYKRKYSQFTLQKVEFQNKTG |
| | | LRNVAKLPKTYYTNAIRFASDTFSGFDEIIKKKQNRLNSIQNRLNFWKEL |
| | | LYNPSNRNEIKIKVVKYAPKTDTREHPHYYSEAEIKGRIKRLEKQLKKFK |
| | | MPKYPEFTSETISLQRELYSWKNPDELKISSITDKNESMNYYGKEYLKRY |
| | | IDLINSQTPQILLEKENNSFYLCFPITKNIEMPKIDDTFEPVGIDWGITRNIA |
| | | VVSILDSKTKKPKFVKFYSAGYILGKRKHYKSLRKHFGQKKRQDKINKL |
| | | GTKEDRFIDSNIHKLAFLIVKEIRNHSNKPIILMENITDNREEAEKSMRQNI |
| | | LLHSVKSRLQNYIAYKALWNNIPTNLVKPEHTSQICNRCGHQDRENRPK |
| | | GSKLFKCVKCNYMSNADFNASINIARKFYIGEYEPFYKDNEKMKSGVNS |
| | | ISM |
| Cas14 | 120 | LKLSEQENITTGVKFKLKLDKETSEGLNDYFDEYGKAINFAIKVIQKELA |
| ortholog 14 | | EDRFAGKVRLDENKKPLLNEDGKKIWDFPNEFCSCGKQVNRYVNGKSL |
| | | CQECYKNKFTEYGIRKRMYSAKGRKAEQDINIKNSTNKISKTHFNYAIRE |
| | | AFILDKSIKKQRKERFRRLREMKKKLQEFIEIRDGNKILCPKIEKQRVERY |
| | | IHPSWINKEKKLEDFRGYSMSNVLGKIKILDRNIKREEKSLKEKGQINFK |
| | | ARRLMLDKSVKFLNDNKISFTISKNLPKEYELDLPEKEKRLNWLKEKIKII |
| | | KNQKPKYAYLLRKDDNFYLQYTLETEFNLKEDYSGIVGIDRGVSHIAVY |
| | | TFVHNNGKNERPLFLNSSEILRLKNLQKERDRFLRRKHNKKRKKSNMRN |
| | | IEKKIQLILHNYSKQIVDFAKNKNAFIVFEKLEKPKKNRSKMSKKSQYKL |
| | | SQFTFKKLSDLVDYKAKREGIKVLYISPEYTSKECSHCGEKVNTQRPFNG |
| | | NSSLFKCNKCGVELNADYNASINIAKKGLNILNSTN |
| Cas14 | 121 | MEESIITGVKFKLRIDKETTKKLNEYFDEYGKAINFAVKIIQKELADDRFA |
| ortholog 15 | | GKAKLDQNKNPILDENGKKIYEFPDEFCSCGKQVNKYVNNKPFCQECY |
| | | KIRFTENGIRKRMYSAKGRKAEHKINILNSTNKISKTHFNYAIREAFILDK |
| | | SIKKQRKKRNERLRESKKRLQQFIDMRDGKREICPTIKGQKVDRFIHPSW |
| | | ITKDKKLEDFRGYTLSIINSKIKILDRNIKREEKSLKEKGQIIFKAKRLMLD |
| | | KSIRFVGDRKVLFTISKTLPKEYELDLPSKEKRLNWLKEKIEIIKNQKPKY |
| | | AYLLRKNIESEKKPNYEYYLQYTLEIKPELKDFYDGAIGIDRGINHIAVCT |
| | | FISNDGKVTPPKFFSSGEILRLKNLQKERDRFLLRKHNKNRKKGNMRVIE |
| | | NKINLILHRYSKQIVDMAKKLNASIVFEELGRIGKSRTKMKKSQRYKLSL |
| | | FIFKKLSDLVDYKSRREGIRVTYVPPEYTSKECSHCGEKVNTQRPFNGNY |
| | | SLFKCNKCGIQLNSDYNASINIAKKGLKIPNST |
| Cas14 | 122 | LWTIVIGDFIEMPKQDLVTTGIKFKLDVDKETRKKLDDYFDEYGKAINFA |
| ortholog 16 | | VKIIQKNLKEDRFAGKIALGEDKKPLLDKDGKKIYNYPNESCSCGNQVR |
| | | RYVNAKPFCVDCYKLKFTENGIRKRMYSARGRKADSDINIKNSTNKISK |
| | | THFNYAIREGFILDKSLKKQRSKRIKKLLELKRKLQEFIDIRQGQMVLCPK |
| | | IKNQRVDKFIHPSWLKRDKKLEEFRGYSLSVVEGKIKIFNRNILREEDSLR |
| | | QRGHVNFKANRIMLDKSVRFLDGGKVNFNLNKGLPKEYLLDLPKKENK |
| | | LSWLNEKISLIKLQKPKYAYLLRREGSFFIQYTIENVPKTFSDYLGAIGIDR |
| | | GISHIAVCTFVSKNGVNKAPVFFSSGEILKLKSLQKQRDLFLRGKHNKIR |
| | | KKSNMRNIDNKINLILHKYSRNIVNLAKSEKAFIVFEKLEKIKKSRFKMS |
| | | KSLQYKLSQFTFKKLSDLVEYKAKIEGIKVDYVPPEYTSKECSHCGEKVD |
| | | TQRPFNGNSSLFKCNKCRVQLNADYNASINIAKKSLNISN |
| Cas14 | 123 | MSKTTISVKLKIIDLSSEKKEFLDNYFNEYAKATTFCQLRIRRLLRNTHW |
| ortholog 17 | | LGKKEKSSKKWIFESGICDLCGENKELVNEDRNSGEPAKICKRCYNGRY |
| | | GNQMIRKLFVSTKKREVQENMDIRRVAKLNNTHYHRIPEEAFDMIKAAD |
| | | TAEKRRKKNVEYDKKRQMEFIEMFNDEKKRAARPKKPNERETRYVHIS |
| | | KLESPSKGYTLNGIKRKIDGMGKKIERAEKGLSRKKIFGYQGNRIKLDSN |
| | | WVRFDLAESEITIPSLFKEMKLRITGPTNVHSKSGQIYFAEWFERINKQPN |
| | | NYCYLIRKTSSNGKYEYYLQYTYEAEVEANKEYAGCLGVDIGCSKLAA |
| | | AVYYDSKNKKAQKPIEIFTNPIKKIKMRREKLIKLLSRVKVRHRRRKLMQ |
| | | LSKTEPIIDYTCHKTARKIVEMANTAKAFISMENLETGIKQKQQARETKK |
| | | QKFYRNMFLFRKLSKLIEYKALLKGIKIVYVKPDYTSQTCSSCGADKEKT |
| | | ERPSQAIFRCLNPTCRYYQRDINADFNAAVNIAKKALNNTEVVTTLL |
| Cas14 | 124 | MARAKNQPYQKLTTTTGIKFKLDLSEEEGKRFDEYFSEYAKAVNFCAKV |
| ortholog 18 | | IYQLRKNLKFAGKKELAAKEWKFEISNCDFCNKQKEIYYKNIANGQKVC |
| | | KGCHRTNFSDNAIRKKMIPVKGRKVESKFNIHNTTKKISGTHRHWAFED |
| | | AADIIESMDKQRKEKQKRLRREKRKLSYFFELFGDPAKRYELPKVGKQR |
| | | VPRYLHKIIDKDSLTKKRGYSLSYIKNKIKISERNIERDEKSLRKASPIAFG |
| | | ARKIKMSKLDPKRAFDLENNVFKIPGKVIKGQYKFFGTNVANEHGKKFY |
| | | KDRISKILAGKPKYFYLLRKKVAESDGNPIFEYYVQWSIDTETPAITSYDN |
| | | ILGIDAGITNLATTVLIPKNLSAEHCSHCGNNHVKPIFTKFFSGKELKAIKI |
| | | KSRKQKYFLRGKHNKLVKIKRIRPIEQKVDGYCHVVSKQIVEMAKERNS |
| | | CIALEKLEKPKKSKFRQRRREKYAVSMFVFKKLATFIKYKAAREGIEIIPV |
| | | EPEGTSYTCSHCKNAQNNQRPYFKPNSKKSWTSMFKCGKCGIELNSDYN |
| | | AAFNIAQKALNMTSA |
| Cas14 | 125 | MDEKHFFCSYCNKELKISKNLINKISKGSIREDEAVSKAISIHNKKEHSLIL |
| ortholog 19 | | GIKFKLFIENKLDKKKLNEYFDNYSKAVTFAARIFDKIRSPYKFIGLKDKN |
| | | TKKWTFPKAKCVFCLEEKEVAYANEKDNSKICTECYLKEFGENGIRKKI |
| | | YSTRGRKVEPKYNIFNSTKELSSTHYNYAIRDAFQLLDALKKQRQKKLK |
| | | SIFNQKLRLKEFEDIFSDPQKRIELSLKPHQREKRYIHLSKSGQESINRGYT |
| | | LRFVRGKIKSLTRNIEREEKSLRKKTPIHFKGNRLMIFPAGIKFDFASNKV |
| | | KISISKNLPNEFNFSGTNVKNEHGKSFFKSRIELIKTQKPKYAYVLRKIKR |
| | | EYSKLRNYEIEKIRLENPNADLCDFYLQYTIETESRNNEEINGIIGIDRGIT |
| | | NLACLVLLKKGDKKPSGVKFYKGNKILGMKIAYRKHLYLLKGKRNKLR |
| | | KQRQIRAIEPKINLILHQISKDIVKIAKEKNFAIALEQLEKPKKARFAQRKK |
| | | EKYKLALFTFKNLSTLIEYKSKREGIPVIYVPPEKTSQMCSHCAINGDEHV |
| | | DTQRPYKKPNAQKPSYSLFKCNKCGIELNADYNAAFNIAQKGLKTLML |
| | | NHSH |
| Cas14 | 126 | MLQTLLVKLDPSKEQYKMLYETMERFNEACNQIAETVFAIHSANKIEVQ |
| ortholog 20 | | KTVYYPIREKFGLSAQLTILAIRKVCEAYKRDKSIKPEFRLDGALVYDQR |
| | | VLSWKGLDKVSLVTLQGRQIIPIKFGDYQKARMDRIRGQADLILVKGVF |
| | | YLCVVVEVSEESPYDPKGVLGVDLGIKNLAVDSDGEVHSGEQTTNTRER |
| | | LDSLKARLQSKGTKSAKRHLKKLSGRMAKFSKDVNHCISKKLVAKAKG |
| | | TLMSIALEDLQGIRDRVTVRKAQRRNLHTWNFGLLRMFVDYKAKIAGV |
| | | PLVFVDPRNTSRTCPSCGHVAKANRPTRDEFRCVSCGFAGAADHIAAMN |
| | | IAFRAEVSQPIVTRFFVQSQAPSFRVG |
| Cas14 | 127 | MDEEPDSAEPNLAPISVKLKLVKLDGEKLAALNDYFNEYAKAVNFCELK |
| ortholog 21 | | MQKIRKNLVNIRGTYLKEKKAWINQTGECCICKKIDELRCEDKNPDING |
| | | KICKKCYNGRYGNQMIRKLFVSTNKRAVPKSLDIRKVARLHNTHYHRIP |
| | | PEAADIIKAIETAERKRRNRILFDERRYNELKDALENEEKRVARPKKPKE |
| | | REVRYVPISKKDTPSKGYTMNALVRKVSGMAKKIERAKRNLNKRKKIE |
| | | YLGRRILLDKNWVRFDFDKSEISIPTMKEFFGEMRFEITGPSNVMSPNGR |
| | | EYFTKWFDRIKAQPDNYCYLLRKESEDETDFYLQYTWRPDAHPKKDYT |
| | | GCLGIDIGGSKLASAVYFDADKNRAKQPIQIFSNPIGKWKTKRQKVIKVL |
| | | SKAAVRHKTKKLESLRNIEPRIDVHCHRIARKIVGMALAANAFISMENLE |
| | | GGIREKQKAKETKKQKFSRNMFVFRKLSKLIEYKALMEGVKVVYIVPDY |
| | | TSQLCSSCGTNNTKRPKQAIFMCQNTECRYFGKNINADFNAAINIAKKAL |
| | | NRKDIVRELS |
| Cas14 | 128 | MEKNNSEQTSITTGIKFKLKLDKETKEKLNNYFDEYGKAINFAVRIIQMQ |
| ortholog 22 | | LNDDRLAGKYKRDEKGKPILGEDGKKILEIPNDFCSCGNQVNHYVNGVS |
| | | FCQECYKKRFSENGIRKRMYSAKGRKAEQDINIKNSTNKISKTHFNYAIR |
| | | EAFNLDKSIKKQREKRFKKLKDMKRKLQEFLEIRDGKRVICPKIEKQKVE |
| | | RYIHPSWINKEKKLEEFRGYSLSIVNSKIKSFDRNIQREEKSLKEKGQINF |
| | | KAQRLMLDKSVKFLKDNKVSFTISKELPKTFELDLPKKEKKLNWLNEKL |
| | | EIIKNQKPKYAYLLRKENNIFLQYTLDSIPEIHSEYSGAVGIDRGVSHIAV |
| | | YTFLDKDGKNERPFFLSSSGILRLKNLQKERDKFLRKKHNKIRKKGNMR |
| | | NIEQKINLILHEYSKQIVNFAKDKNAFIVFELLEKPKKSRERMSKKIQYKL |
| | | SQFTFKKLSDLVDYKAKREGIKVIYVEPAYTSKDCSHCGERVNTQRPFN |
| | | GNFSLFKCNKCGIVLNSDYNASLNIARKGLNISAN |
| Cas14 | 129 | MAEEKFFFCEKCNKDIKIPKNYINKQGAEEKARAKHEHRVHALILGIKFK |
| ortholog 23 | | IYPKKEDISKLNDYFDEYAKAVTFTAKIVDKLKAPFLFAGKRDKDTSKK |
| | | KWVFPVDKCSFCKEKTEINYRTKQGKNICNSCYLTEFGEQGLLEKIYAT |
| | | KGRKVSSSFNLFNSTKKLTGTHNNYVVKESLQLLDALKKQRSKRLKKLS |
| | | NTRRKLKQFEEMFEKEDKRFQLPLKEKQRELRFIHVSQKDRATEFKGYT |
| | | MNKIKSKIKVLRRNIEREQRSLNRKSPVFFRGTRIRLSPSVQFDDKDNKIK |
| | | LTLSKELPKEYSFSGLNVANEHGRKFFAEKLKLIKENKSKYAYLLRRQV |
| | | NKNNKKPIYDYYLQYTVEFLPNIITNYNGILGIDRGINTLACIVLLENKKE |
| | | KPSFVKFFSGKGILNLKNKRRKQLYFLKGVHNKYRKQQKIRPIEPRIDQIL |
| | | HDISKQIIDLAKEKRVAISLEQLEKPQKPKFRQSRKAKYKLSQFNFKTLSN |
| | | YIDYKAKKEGIRVIYIAPEMTSQNCSRCAMKNDLHVNTQRPYKNTSSLF |
| | | KCNKCGVELNADYNAAFNIAQKGLKILNS |
| Cas14 | 130 | MISLKLKLLPDEEQKKLLDEMFWKWASICTRVGFGRADKEDLKPPKDA |
| ortholog 24 | | EGVWFSLTQLNQANTDINDLREAMKHQKHRLEYEKNRLEAQRDDTQD |
| | | ALKNPDRREISTKRKDLFRPKASVEKGFLKLKYHQERYWVRRLKEINKL |
| | | IERKTKTLIKIEKGRIKFKATRITLHQGSFKIRFGDKPAFLIKALSGKNQID |
| | | APFVVVPEQPICGSVVNSKKYLDEITTNFLAYSVNAMLFGLSRSEEMLLK |
| | | AKRPEKIKKKEEKLAKKQSAFENKKKELQKLLGRELTQQEEAIIEETRNQ |
| | | FFQDFEVKITKQYSELLSKIANELKQKNDFLKVNKYPILLRKPLKKAKSK |
| | | KINNLSPSEWKYYLQFGVKPLLKQKSRRKSRNVLGIDRGLKHLLAVTVL |
| | | EPDKKTFVWNKLYPNPITGWKWRRRKLLRSLKRLKRRIKSQKHETIHEN |
| | | QTRKKLKSLQGRIDDLLHNISRKIVETAKEYDAVIVVEDLQSMRQHGRS |
| | | KGNRLKTLNYALSLFDYANVMQLIKYKAGIEGIQIYDVKPAGTSQNCAY |
| | | CLLAQRDSHEYKRSQENSKIGVCLNPNCQNHKKQIDADLNAARVIASCY |
| | | ALKINDSQPFGTRKRFKKRTTN |
| Cas14 | 131 | METLSLKLKLNPSKEQLLVLDKMFWKWASICTRLGLKKAEMSDLEPPK |
| ortholog 25 | | DAEGVWFSKTQLNQANTDVNDLRKAMQHQGKRIEYELDKVENRRNEI |
| | | QEMLEKPDRRDISPNRKDLFRPKAAVEKGYLKLKYHKLGYWSKELKTA |
| | | NKLIERKRKTLAKIDAGKMKFKPTRISLHTNSFRIKFGEEPKIALSTTSKH |
| | | EKIELPLITSLQRPLKTSCAKKSKTYLDAAILNFLAYSTNAALFGLSRSEE |
| | | MLLKAKKPEKIEKRDRKLATKRESFDKKLKTLEKLLERKLSEKEKSVFK |
| | | RKQTEFFDKFCITLDETYVEALHRIAEELVSKNKYLEIKKYPVLLRKPESR |
| | | LRSKKLKNLKPEDWTYYIQFGFQPLLDTPKPIKTKTVLGIDRGVRHLLAV |
| | | SIFDPRTKTFTFNRLYSNPIVDWKWRRRKLLRSIKRLKRRLKSEKHVHLH |
| | | ENQFKAKLRSLEGRIEDHFHNLSKEIVDLAKENNSVIVVENLGGMRQHG |
| | | RGRGKWLKALNYALSHFDYAKVMQLIKYKAELAGVFVYDVAPAGTSI |
| | | NCAYCLLNDKDASNYTRGKVINGKKNTKIGECKTCKKEFDADLNAARV |
| | | IALCYEKRLNDPQPFGTRKQFKPKKP |
| Cas14 | 132 | MKALKLQLIPTRKQYKILDEMFWKWASLANRVSQKGESKETLAPKKDI |
| ortholog 26 | | QKIQFNATQLNQIEKDIKDLRGAMKEQQKQKERLLLQIQERRSTISEMLN |
| | | DDNNKERDPHRPLNFRPKGWRKFHTSKHWVGELSKILRQEDRVKKTIER |
| | | IVAGKISFKPKRIGIWSSNYKINFFKRKISINPLNSKGFELTLMTEPTQDLIG |
| | | KNGGKSVLNNKRYLDDSIKSLLMFALHSRFFGLNNTDTYLLGGKINPSL |
| | | VKYYKKNQDMGEFGREIVEKFERKLKQEINEQQKKIIMSQIKEQYSNRD |
| | | SAFNKDYLGLINEFSEVFNQRKSERAEYLLDSFEDKIKQIKQEIGESLNISD |
| | | WDFLIDEAKKAYGYEEGFTEYVYSKRYLEILNKIVKAVLITDIYFDLRKY |
| | | PILLRKPLDKIKKISNLKPDEWSYYIQFGYDSINPVQLMSTDKFLGIDRGL |
| | | THLLAYSVFDKEKKEFIINQLEPNPIMGWKWKLRKVKRSLQHLERRIRA |
| | | QKMVKLPENQMKKKLKSIEPKIEVHYHNISRKIVNLAKDYNASIVVESLE |
| | | GGGLKQHGRKKNARNRSLNYALSLFDYGKIASLIKYKADLEGVPMYEV |
| | | LPAYTSQQCAKCVLEKGSFVDPEIIGYVEDIGIKGSLLDSLFEGTELSSIQV |
| | | LKKIKNKIELSARDNHNKEINLILKYNFKGLVIVRGQDKEEIAEHPIKEIN |
| | | GKFAILDFVYKRGKEKVGKKGNQKVRYTGNKKVGYCSKHGQVDADLN |
| | | ASRVIALCKYLDINDPILFGEQRKSFK |
| Cas14 | 133 | MVTRAIKLKLDPTKNQYKLLNEMFWKWASLANRFSQKGASKETLAPK |
| ortholog 27 | | DGTQKIQFNATQLNQIKKDVDDLRGAMEKQGKQKERLLIQIQERLLTISE |
| | | ILRDDSKKEKDPHRPQNFRPFGWRRFHTSAYWSSEASKLTRQVDRVRRT |
| | | IERIKAGKINFKPKRIGLWSSTYKINFLKKKINISPLKSKSFELDLITEPQQK |
| | | IIGKEGGKSVANSKKYLDDSIKSLLIFAIKSRLFGLNNKDKPLFENIITPNL |
| | | VRYHKKGQEQENFKKEVIKKFENKLKKEISQKQKEIIFSQIERQYENRDA |
| | | TFSEDYLRAISEFSEIFNQRKKERAKELLNSFNEKIRQLKKEVNGNISEED |
| | | LKILEVEAEKAYNYENGFIEWEYSEQFLGVLEKIARAVLISDNYFDLKKY |
| | | PILIRKPTNKSKKITNLKPEEWDYYIQFGYGLINSPMKIETKNFMGIDRGL |
| | | THLLAYSIFDRDSEKFTINQLELNPIKGWKWKLRKVKRSLQHLERRMRA |
| | | QKGVKLPENQMKKRLKSIEPKIESYYHNLSRKIVNLAKANNASIVVESLE |
| | | GGGLKQHGRKKNSRHRALNYALSLFDYGKIASLIKYKSDLEGVPMYEV |
| | | LPAYTSQQCAKCVLKKGSFVEPEIIGYIEEIGFKENLLTLLFEDTGLSSVQ |
| | | VLKKSKNKMTLSARDKEGKMVDLVLKYNFKGLVISQEKKKEEIVEFPIK |
| | | EIDGKFAVLDSAYKRGKERISKKGNQKLVYTGNKKVGYCSVHGQVDAD |
| | | LNASRVIALCKYLGINEPIVFGEQRKSFK |
| Cas14 | 134 | LDLITEPIQPHKSSSLRSKEFLEYQISDFLNFSLHSLFFGLASNEGPLVDFKI |
| ortholog 28 | | YDKIVIPKPEERFPKKESEEGKKLDSFDKRVEEYYSDKLEKKIERKLNTEE |
| | | KNVIDREKTRIWGEVNKLEEIRSIIDEINEIKKQKHISEKSKLLGEKWKKV |
| | | NNIQETLLSQEYVSLISNLSDELTNKKKELLAKKYSKFDDKIKKIKEDYG |
| | | LEFDENTIKKEGEKAFLNPDKFSKYQFSSSYLKLIGEIARSLITYKGFLDL |
| | | NKYPIIFRKPINKVKKIHNLEPDEWKYYIQFGYEQINNPKLETENILGIDR |
| | | GLTHILAYSVFEPRSSKFILNKLEPNPIEGWKWKLRKLRRSIQNLERRWR |
| | | AQDNVKLPENQMKKNLRSIEDKVENLYHNLSRKIVDLAKEKNACIVFEK |
| | | LEGQGMKQHGRKKSDRLRGLNYKLSLFDYGKIAKLIKYKAEIEGIPIYRI |
| | | DSAYTSQNCAKCVLESRRFAQPEEISCLDDFKEGDNLDKRILEGTGLVEA |
| | | KIYKKLLKEKKEDFEIEEDIAMFDTKKVIKENKEKTVILDYVYTRRKEIIG |
| | | TNHKKNIKGIAKYTGNTKIGYCMKHGQVDADLNASRTIALCKNFDINNP |
| | | EIWK |
| Cas14 | 135 | MSDESLVSSEDKLAIKIKIVPNAEQAKMLDEMFKKWSSICNRISRGKEDI |
| ortholog 29 | | ETLRPDEGKELQFNSTQLNSATMDVSDLKKAMARQGERLEAEVSKLRG |
| | | RYETIDASLRDPSRRHTNPQKPSSFYPSDWDISGRLTPRFHTARHYSTELR |
| | | KLKAKEDKMLKTINKIKNGKIVFKPKRITLWPSSVNMAFKGSRLLLKPFA |
| | | NGFEMELPIVISPQKTADGKSQKASAEYMRNALLGLAGYSINQLLFGMN |
| | | RSQKMLANAKKPEKVEKFLEQMKNKDANFDKKIKALEGKWLLDRKLK |
| | | ESEKSSIAVVRTKFFKSGKVELNEDYLKLLKHMANEILERDGFVNLNKY |
| | | PILSRKPMKRYKQKNIDNLKPNMWKYYIQFGYEPIFERKASGKPKNIMGI |
| | | DRGLTHLLAVAVFSPDQQKFLFNHLESNPIMHWKWKLRKIRRSIQHMER |
| | | RIRAEKNKHIHEAQLKKRLGSIEEKTEQHYHIVSSKIINWAIEYEAAIVLES |
| | | LSHMKQRGGKKSVRTRALNYALSLFDYEKVARLITYKARIRGIPVYDVL |
| | | PGMTSKTCATCLLNGSQGAYVRGLETTKAAGKATKRKNMKIGKCMVC |
| | | NSSENSMIDADLNAARVIAICKYKNLNDPQPAGSRKVFKRF |
| Cas14 | 136 | MLALKLKIMPTEKQAEILDAMFWKWASICSRIAKMKKKVSVKENKKEL |
| ortholog 30 | | SKKIPSNSDIWFSKTQLCQAEVDVGDHKKALKNFEKRQESLLDELKYKV |
| | | KAINEVINDESKREIDPNNPSKFRIKDSTKKGNLNSPKFFTLKKWQKILQE |
| | | NEKRIKKKESTIEKLKRGNIFFNPTKISLHEEEYSINFGSSKLLLNCFYKYN |
| | | KKSGINSDQLENKFNEFQNGLNIICSPLQPIRGSSKRSFEFIRNSIINFLMYS |
| | | LYAKLFGIPRSVKALMKSNKDENKLKLEEKLKKKKSSFNKTVKEFEKMI |
| | | GRKLSDNESKILNDESKKFFEIIKSNNKYIPSEEYLKLLKDISEEIYNSNIDF |
| | | KPYKYSILIRKPLSKFKSKKLYNLKPTDYKYYLQLSYEPFSKQLIATKTIL |
| | | GIDRGLKHLLAVSVFDPSQNKFVYNKLIKNPVFKWKKRYHDLKRSIRNR |
| | | ERRIRALTGVHIHENQLIKKLKSMKNKINVLYHNVSKNIVDLAKKYESTI |
| | | VLERLENLKQHGRSKGKRYKKLNYVLSNFDYKKIESLISYKAKKEGVPV |
| | | SNINPKYTSKTCAKCLLEVNQLSELKNEYNRDSKNSKIGICNIHGQIDAD |
| | | LNAARVIALCYSKNLNEPHFK |
| Cas14 | 137 | VINLFGYKFALYPNKTQEELLNKHLGECGWLYNKAIEQNEYYKADSNIE |
| ortholog 31 | | EAQKKFELLPDKNSDEAKVLRGNISKDNYVYRTLVKKKKSEINVQIRKA |
| | | VVLRPAETIRNLAKVKKKGLSVGRLKFIPIREWDVLPFKQSDQIRLEENY |
| | | LILEPYGRLKFKMHRPLLGKPKTFCIKRTATDRWTISFSTEYDDSNMRKN |
| | | DGGQVGIDVGLKTHLRLSNENPDEDPRYPNPKIWKRYDRRLTILQRRISK |
| | | SKKLGKNRTRLRLRLSRLWEKIRNSRADLIQNETYEILSENKLIAIEDLNV |
| | | KGMQEKKDKKGRKGRTRAQEKGLHRSISDAAFSEFRRVLEYKAKRFGS |
| | | EVKPVSAIDSSKECHNCGNKKGMPLESRIYECPKCGLKIDRDLNSAKVIL |
| | | ARATGVRPGSNARADTKISATAGASVQTEGTVSEDFRQQMETSDQKPM |
| | | QGEGSKEPPMNPEHKSSGRGSKHVNIGCKNKVGLYNEDENSRSTEKQIM |
| | | DENRSTTEDMVEIGALHSPVLTT |
| Cas14 | 138 | MIASIDYEAVSQALIVFEFKAKGKDSQYQAIDEAIRSYRFIRNSCLRYWM |
| ortholog 32 | | DNKKVGKYDLNKYCKVLAKQYPFANKLNSQARQSAAECSWSAISRFYD |
| | | NCKRKVSGKKGFPKFKKHARSVEYKTSGWKLSENRKAITFTDKNGIGKL |
| | | KLKGTYDLHFSQLEDMKRVRLVRRADGYYVQFCISVDVKVETEPTGKA |
| | | IGLDVGIKYFLADSSGNTIENPQFYRKAEKKLNRANRRKSKKYIRGVKPQ |
| | | SKNYHKARCRYARKHLRVSRQRKEYCKRVAYCVIHSNDVVAYEDLNV |
| | | KGMVKNRHLAKSISDVAWSTFRHWLEYFAIKYGKLTIPVAPHNTSQNCS |
| | | NCDKKVPKSLSTRTHICHHCGYSEDRDVNAAKNILKKALSTVGQTGSLK |
| | | LGEIEPLLVLEQSCTRKFDL |
| Cas14 | 139 | LAEENTLHLTLAMSLPLNDLPENRTRSELWRRQWLPQKKLSLLLGVNQS |
| ortholog 33 | | VRKAAADCLRWFEPYQELLWWEPTDPDGKKLLDKEGRPIKRTAGHMR |
| | | VLRKLEEIAPFRGYQLGSAVKNGLRHKVADLLLSYAKRKLDPQFTDKTS |
| | | YPSIGDQFPIVWTGAFVCYEQSITGQLYLYLPLFPRGSHQEDITNNYDPDR |
| | | GPALQVFGEKEIARLSRSTSGLLLPLQFDKWGEATFIRGENNPPTWKATH |
| | | RRSDKKWLSEVLLREKDFQPKRVELLVRNGRIFVNVACEIPTKPLLEVEN |
| | | FMGVSFGLEHLVTVVVINRDGNVVHQRQEPARRYEKTYFARLERLRRR |
| | | GGPFSQELETFHYRQVAQIVEEALRFKSVPAVEQVGNIPKGRYNPRLNLR |
| | | LSYWPFGKLADLTSYKAVKEGLPKPYSVYSATAKMLCSTCGAANKEGD |
| | | QPISLKGPTVYCGNCGTRHNTGENTALNLARRAQELFVKGVVAR |
| Cas14 | 140 | MSQSLLKWHDMAGRDKDASRSLQKSAVEGVLLHLTASHRVALEMLEK |
| ortholog 34 | | SVSQTVAVTMEAAQQRLVIVLEDDPTKATSRKRVISADLQFTREEFGSLP |
| | | NWAQKLASTCPEIATKYADKHINSIRIAWGVAKESTNGDAVEQKLQWQI |
| | | RLLDVTMFLQQLVLQLADKALLEQIPSSIRGGIGQEVAQQVTSHIQLLDS |
| | | GTVLKAELPTISDRNSELARKQWEDAIQTVCTYALPFSRERARILDPGKY |
| | | AAEDPRGDRLINIDPMWARVLKGPTVKSLPLLFVSGSSIRIVKLTLPRKH |
| | | AAGHKHTFTATYLVLPVSREWINSLPGTVQEKVQWWKKPDVLATQELL |
| | | VGKGALKKSANTLVIPISAGKKRFFNHILPALQRGFPLQWQRIVGRSYRR |
| | | PATHRKWFAQLTIGYTNPSSLPEMALGIHFGMKDILWWALADKQGNILK |
| | | DGSIPGNSILDFSLQEKGKIERQQKAGKNVAGKKYGKSLLNATYRVVNG |
| | | VLEFSKGISAEHASQPIGLGLETIRFVDKASGSSPVNARHSNWNYGQLSGI |
| | | FANKAGPAGFSVTEITLKKAQRDLSDAEQARVLAIEATKRFASRIKRLAT |
| | | KRKDDTLFV |
| Cas14 | 141 | VEPVEKERFYYRTYTFRLDGQPRTQNLTTQSGWGLLTKAVLDNTKHYW |
| ortholog 35 | | EIVHHARIANQPIVFENPVIDEQGNPKLNKLGQPRFWKRPISDIVNQLRAL |
| | | FENQNPYQLGSSLIQGTYWDVAENLASWYALNKEYLAGTATWGEPSFP |
| | | EPHPLTEINQWMPLTFSSGKVVRLLKNASGRYFIGLPILGENNPCYRMRT |
| | | IEKLIPCDGKGRVTSGSLILFPLVGIYAQQHRRMTDICESIRTEKGKLAWA |
| | | QVSIDYVREVDKRRRMRRTRKSQGWIQGPWQEVFILRLVLAHKAPKLY |
| | | KPRCFAGISLGPKTLASCVILDQDERVVEKQQWSGSELLSLIHQGEERLR |
| | | SLREQSKPTWNAAYRKQLKSLINTQVFTIVTFLRERGAAVRLESIARVRK |
| | | STPAPPVNFLLSHWAYRQITERLKDLAIRNGMPLTHSNGSYGVRFTCSQC |
| | | GATNQGIKDPTKYKVDIESETFLCSICSHREIAAVNTATNLAKQLLDE |
| Cas14 | 142 | MNDTETSETLTSHRTVCAHLHVVGETGSLPRLVEAALAELITLNGRATQ |
| ortholog 36 | | ALLSLAKNGLVLRRDKEENLIAAELTLPCRKNKYADVAAKAGEPILATRI |
| | | NNKGKLVTKKWYGEGNSYHIVRFTPETGMFTVRVFDRYAFDEELLHLH |
| | | SEVVFGSDLPKGIKAKTDSLPANFLQAVFTSFLELPFQGFPDIVVKPAMK |
| | | QAAEQLLSYVQLEAGENQQAEYPDTNERDPELRLVEWQKSLHELSVRT |
| | | EPFEFVRARDIDYYAETDRRGNRFVNITPEWTKFAESPFARRLPLKIPPEF |
| | | CILLRRKTEGHAKIPNRIYLGLQIFDGVTPDSTLGVLATAEDGKLFWWHD |
| | | HLDEFSNLEGKPEPKLKNKPQLLMVSLEYDREQRFEESVGGDRKICLVT |
| | | LKETRNFRRGWNGRILGIHFQHNPVITWALMDHDAEVLEKGFIEGNAFL |
| | | GKALDKQALNEYLQKGGKWVGDRSFGNKLKGITHTLASLIVRLAREKD |
| | | AWIALEEISWVQKQSADSVANHEIVEQPHHSLTR |
| Cas14 | 143 | MNDTETSETLTSHRTVCAHLHVVGETGSLPRLVEAALAELITLNGRATQ |
| ortholog 37 | | ALLSLAKNGLVLRRDKEENLIAAELTLPCRKNKYADVAAKAGEPILATRI |
| | | NNKGKLVTKKWYGEGNSYHIVRFTPETGMFTVRVFDRYAFDEELLHLH |
| | | SEVVFGSDLPKGIKAKTDSLPANFLQAVFTSFLELPFQGFPDIVVKPAMK |
| | | QAAEQLLSYVQLEAGENQQAEYPDTNERDPELRLVEWQKSLHELSVRT |
| | | EPFEFVRARDIDYYAETDRRGNRFVNITPEWTKFAESPFARRLPLKIPPEF |
| | | CILLRRKTEGHAKIPNRIYLGLQIFDGVTPDSTLGVLATAEDGKLFWWHD |
| | | HLDEFSNLEGKPEPKLKNKPQLLMVSLEYDREQRFEESVGGDRKICLVT |
| | | LKETRNFRRGRHGHTRTDRLPAGNTLWRADFATSAEVAAPKWNGRILG |
| | | IHFQHNPVITWALMDHDAEVLEKGFIEGNAFLGKALDKQALNEYLQKG |
| | | GKWVGDRSFGNKLKGITHTLASLIVRLAREKDAWIALEEISWVQKQSAD |
| | | SVANRRFSMWNYSRLATLIEWLGTDIATRDCGTAAPLAHKVSDYLTHFT |
| | | CPECGACRKAGQKKEIADTVRAGDILTCRKCGFSGPIPDNFIAEFVAKKA |
| | | LERMLKKKPV |
| Cas14 | 144 | MAKRNFGEKSEALYRAVRFEVRPSKEELSILLAVSEVLRMLFNSALAER |
| ortholog 38 | | QQVFTEFIASLYAELKSASVPEEISEIRKKLREAYKEHSISLFDQINALTAR |
| | | RVEDEAFASVTRNWQEETLDALDGAYKSFLSLRRKGDYDAHSPRSRDS |
| | | GFFQKIPGRSGFKIGEGRIALSCGAGRKLSFPIPDYQQGRLAETTKLKKFE |
| | | LYRDQPNLAKSGRFWISVVYELPKPEATTCQSEQVAFVALGASSIGVVS |
| | | QRGEEVIALWRSDKHWVPKIEAVEERMKRRVKGSRGWLRLLNSGKRR |
| | | MHMISSRQHVQDEREIVDYLVRNHGSHFVVTELVVRSKEGKLADSSKPE |
| | | RGGSLGLNWAAQNTGSLSRLVRQLEEKVKEHGGSVRKHKLTLTEAPPA |
| | | RGAENKLWMARKLRESFLKEV |
| Cas14 | 145 | LAKNDEKELLYQSVKFEIYPDESKIRVLTRVSNILVLVWNSALGERRARF |
| ortholog 39 | | ELYIAPLYEELKKFPRKSAESNALRQKIREGYKEHIPTFFDQLKKLLTPMR |
| | | KEDPALLGSVPRAYQEETLNTLNGSFVSFMTLRRNNDMDAKPPKGRAE |
| | | DRFHEISGRSGFKIDGSEFVLSTKEQKLRFPIPNYQLEKLKEAKQIKKFTL |
| | | YQSRDRRFWISIAYEIELPDQRPFNPEEVIYIAFGASSIGVISPEGEKVIDFW |
| | | RPDKHWKPKIKEVENRMRSCKKGSRAWKKRAAARRKMYAMTQRQQK |
| | | LNHREIVASLLRLGFHFVVTEYTVRSKPGKLADGSNPKRGGAPQGFNWS |
| | | AQNTGSFGEFILWLKQKVKEQGGTVQTFRLVLGQSERPEKRGRDNKIEM |
| | | VRLLREKYLESQTIVV |
| Cas14 | 146 | MAKGKKKEGKPLYRAVRFEIFPTSDQITLFLRVSKNLQQVWNEAWQER |
| ortholog 40 | | QSCYEQFFGSIYERIGQAKKRAQEAGFSEVWENEAKKGLNKKLRQQEIS |
| | | MQLVSEKESLLQELSIAFQEHGVTLYDQINGLTARRIIGEFALIPRNWQEE |
| | | TLDSLDGSFKSFLALRKNGDPDAKPPRQRVSENSFYKIPGRSGFKVSNGQ |
| | | IYLSFGKIGQTLTSVIPEFQLKRLETAIKLKKFELCRDERDMAKPGRFWIS |
| | | VAYEIPKPEKVPVVSKQITYLAIGASRLGVVSPKGEFCLNLPRSDYHWKP |
| | | QINALQERLEGVVKGSRKWKKRMAACTRMFAKLGHQQKQHGQYEVV |
| | | KKLLRHGVHFVVTELKVRSKPGALADASKSDRKGSPTGPNWSAQNTGN |
| | | IARLIQKLTDKASEHGGTVIKRNPPLLSLEERQLPDAQRKIFIAKKLREEFL |
| | | ADQK |
| Cas14 | 147 | MAKREKKDDVVLRGTKMRIYPTDRQVTLMDMWRRRCISLWNLLLNLE |
| ortholog 41 | | TAAYGAKNTRSKLGWRSIWARVVEENHAKALIVYQHGKCKKDGSFVL |
| | | KRDGTVKHPPRERFPGDRKILLGLFDALRHTLDKGAKCKCNVNQPYALT |
| | | RAWLDETGHGARTADIIAWLKDFKGECDCTAISTAAKYCPAPPTAELLT |
| | | KIKRAAPADDLPVDQAILLDLFGALRGGLKQKECDHTHARTVAYFEKHE |
| | | LAGRAEDILAWLIAHGGTCDCKIVEEAANHCPGPRLFIWEHELAMIMAR |
| | | LKAEPRTEWIGDLPSHAAQTVVKDLVKALQTMLKERAKAAAGDESARK |
| | | TGFPKFKKQAYAAGSVYFPNTTMFFDVAAGRVQLPNGCGSMRCEIPRQ |
| | | LVAELLERNLKPGLVIGAQLGLLGGRIWRQGDRWYLSCQWERPQPTLLP |
| | | KTGRTAGVKIAASIVFTTYDNRGQTKEYPMPPADKKLTAVHLVAGKQN |
| | | SRALEAQKEKEKKLKARKERLRLGKLEKGHDPNALKPLKRPRVRRSKLF |
| | | YKSAARLAACEAIERDRRDGFLHRVTNEIVHKFDAVSVQKMSVAPMMR |
| | | RQKQKEKQIESKKNEAKKEDNGAAKKPRNLKPVRKLLRHVAMARGRQ |
| | | FLEYKYNDLRGPGSVLIADRLEPEVQECSRCGTKNPQMKDGRRLLRCIG |
| | | VLPDGTDCDAVLPRNRNAARNAEKRLRKHREAHNA |
| Cas14 | 148 | MNEVLPIPAVGEDAADTIMRGSKMRIYPSVRQAATMDLWRRRCIQLWN |
| ortholog 42 | | LLLELEQAAYSGENRRTQIGWRSIWATVVEDSHAEAVRVAREGKKRKD |
| | | GTFRKAPSGKEIPPLDPAMLAKIQRQMNGAVDVDPKTGEVTPAQPRLFM |
| | | WEHELQKIMARLKQAPRTHWIDDLPSHAAQSVVKDLIKALQAMLRERK |
| | | KRASGIGGRDTGFPKFKKNRYAAGSVYFANTQLRFEAKRGKAGDPDAV |
| | | RGEFARVKLPNGVGWMECRMPRHINAAHAYAQATLMGGRIWRQGEN |
| | | WYLSCQWKMPKPAPLPRAGRTAAIKIAAAIPITTVDNRGQTREYAMPPI |
| | | DRERIAAHAAAGRAQSRALEARKRRAKKREAYAKKRHAKKLERGIAAK |
| | | PPGRARIKLSPGFYAAAAKLAKLEAEDANAREAWLHEITTQIVRNFDVIA |
| | | VPRMEVAKLMKKPEPPEEKEEQVKAPWQGKRRSLKAARVMMRRTAM |
| | | ALIQTTLKYKAVDLRGPQAYEEIAPLDVTAAACSGCGVLKPEWKMARA |
| | | KGREIMRCQEPLPGGKTCNTVLTYTRNSARVIGRELAVRLAERQKA |
| Cas14 | 149 | MTTQKTYNFCFYDQRFFELSKEAGEVYSRSLEEFWKIYDETGVWLSKFD |
| ortholog 43 | | LQKHMRNKLERKLLHSDSFLGAMQQVHANLASWKQAKKVVPDACPPR |
| | | KPKFLQAILFKKSQIKYKNGFLRLTLGTEKEFLYLKWDINIPLPIYGSVTY |
| | | SKTRGWKINLCLETEVEQKNLSENKYLSIDLGVKRVATIFDGENTITLSG |
| | | KKFMGLMHYRNKLNGKTQSRLSHKKKGSNNYKKIQRAKRKTTDRLLNI |
| | | QKEMLHKYSSFIVNYAIRNDIGNIIIGDNSSTHDSPNMRGKTNQKISQNPE |
| | | QKLKNYIKYKFESISGRVDIVPEPYTSRKCPHCKNIKKSSPKGRTYKCKK |
| | | CGFIFDRDGVGAINIYNENVSFGQIISPGRIRSLTEPIGMKFHNEIYFKSYV |
| | | AA |
| Cas14 | 150 | MSVRSFQARVECDKQTMEHLWRTHKVFNERLPEIIKILFKMKRGECGQN |
| ortholog 44 | | DKQKSLYKSISQSILEANAQNADYLLNSVSIKGWKPGTAKKYRNASFTW |
| | | ADDAAKLSSQGIHVYDKKQVLGDLPGMMSQMVCRQSVEAISGHIELTK |
| | | KWEKEHNEWLKEKEKWESEDEHKKYLDLREKFEQFEQSIGGKITKRRG |
| | | RWHLYLKWLSDNPDFAAWRGNKAVINPLSEKAQIRINKAKPNKKNSVE |
| | | RDEFFKANPEMKALDNLHGYYERNFVRRRKTKKNPDGFDHKPTFTLPH |
| | | PTIHPRWFVFNKPKTNPEGYRKLILPKKAGDLGSLEMRLLTGEKNKGNY |
| | | PDDWISVKFKADPRLSLIRPVKGRRVVRKGKEQGQTKETDSYEFFDKHL |
| | | KKWRPAKLSGVKLIFPDKTPKAAYLYFTCDIPDEPLTETAKKIQWLETGD |
| | | VTKKGKKRKKKVLPHGLVSCAVDLSMRRGTTGFATLCRYENGKIHILRS |
| | | RNLWVGYKEGKGCHPYRWTEGPDLGHIAKHKREIRILRSKRGKPVKGE |
| | | ESHIDLQKHIDYMGEDRFKKAARTIVNFALNTENAASKNGFYPRADVLL |
| | | LENLEGLIPDAEKERGINRALAGWNRRHLVERVIEMAKDAGFKRRVFEI |
| | | PPYGTSQVCSKCGALGRRYSIIRENNRREIRFGYVEKLFACPNCGYCANA |
| | | DHNASVNLNRRFLIEDSFKSYYDWKRLSEKKQKEEIETIESKLMDKLCA |
| | | MHKISRGSISK |
| Cas14 | 151 | MHLWRTHCVFNQRLPALLKRLFAMRRGEVGGNEAQRQVYQRVAQFVL |
| ortholog 45 | | ARDAKDSVDLLNAVSLRKRSANSAFKKKATISCNGQAREVTGEEVFAE |
| | | AVALASKGVFAYDKDDMRAGLPDSLFQPLTRDAVACMRSHEELVATW |
| | | KKEYREWRDRKSEWEAEPEHALYLNLRPKFEEGEAARGGRFRKRAERD |
| | | HAYLDWLEANPQLAAWRRKAPPAVVPIDEAGKRRIARAKAWKQASVR |
| | | AEEFWKRNPELHALHKIHVQYLREFVRPRRTRRNKRREGFKQRPTFTMP |
| | | DPVRHPRWCLFNAPQTSPQGYRLLRLPQSRRTVGSVELRLLTGPSDGAG |
| | | FPDAWVNVRFKADPRLAQLRPVKVPRTVTRGKNKGAKVEADGFRYYD |
| | | DQLLIERDAQVSGVKLLFRDIRMAPFADKPIEDRLLSATPYLVFAVEIKD |
| | | EARTERAKAIRFDETSELTKSGKKRKTLPAGLVSVAVDLDTRGVGFLTR |
| | | AVIGVPEIQQTHHGVRLLQSRYVAVGQVEARASGEAEWSPGPDLAHIAR |
| | | HKREIRRLRQLRGKPVKGERSHVRLQAHIDRMGEDRFKKAARKIVNEAL |
| | | RGSNPAAGDPYTRADVLLYESLETLLPDAERERGINRALLRWNRAKLIE |
| | | HLKRMCDDAGIRHFPVSPFGTSQVCSKCGALGRRYSLARENGRAVIRFG |
| | | WVERLFACPNPECPGRRPDRPDRPFTCNSDHNASVNLHRVFALGDQAV |
| | | AAFRALAPRDSPARTLAVKRVEDTLRPQLMRVHKLADAGVDSPF |
| Cas14 | 152 | MATLVYRYGVRAHGSARQQDAVVSDPAMLEQLRLGHELRNALVGVQH |
| ortholog 46 | | RYEDGKRAVWSGFASVAAADHRVTTGETAVAELEKQARAEHSADRTA |
| | | ATRQGTAESLKAARAAVKQARADRKAAMAAVAEQAKPKIQALGDDRD |
| | | AEIKDLYRRFCQDGVLLPRCGRCAGDLRSDGDCTDCGAAHEPRKLYWA |
| | | TYNAIREDHQTAVKLVEAKRKAGQPARLRFRRWTGDGTLTVQLQRMH |
| | | GPACRCVTCAEKLTRRARKTDPQAPAVAADPAYPPTDPPRDPALLASGQ |
| | | GKWRNVLQLGTWIPPGEWSAMSRAERRRVGRSHIGWQLGGGRQLTLP |
| | | VQLHRQMPADADVAMAQLTRVRVGGRHRMSVALTAKLPDPPQVQGLP |
| | | PVALHLGWRQRPDGSLRVATWACPQPLDLPPAVADVVVSHGGRWGEV |
| | | IMPARWLADAEVPPRLLGRRDKAMEPVLEALADWLEAHTEACTARMTP |
| | | ALVRRWRSQGRLAGLTNRWRGQPPTGSAEILTYLEAWRIQDKLLWERE |
| | | SHLRRRLAARRDDAWRRVASWLARHAGVLVVDDADIAELRRRDDPAD |
| | | TDPTMPASAAQAARARAALAAPGRLRHLATITATRDGLGVHTVASAGL |
| | | TRLHRKCGHQAQPDPRYAASAVVTCPGCGNGYDQDYNAAMLMLDRQ |
| | | QQP |
| Cas14 | 153 | MSRVELHRAYKFRLYPTPAQVAELAEWERQLRRLYNLAHSQRLAAMQ |
| ortholog 47 | | RHVRPKSPGVLKSECLSCGAVAVAEIGTDGKAKKTVKHAVGCSVLECR |
| | | SCGGSPDAEGRTAHTAACSFVDYYRQGREMTQLLEEDDQLARVVCSAR |
| | | QETLRDLEKAWQRWHKMPGFGKPHFKKRIDSCRIYFSTPKSWAVDLGY |
| | | LSFTGVASSVGRIKIRQDRVWPGDAKFSSCHVVRDVDEWYAVFPLTFTK |
| | | EIEKPKGGAVGINRGAVHAIADSTGRVVDSPKFYARSLGVIRHRARLLDR |
| | | KVPFGRAVKPSPTKYHGLPKADIDAAAARVNASPGRLVYEARARGSIAA |
| | | AEAHLAALVLPAPRQTSQLPSEGRNRERARRFLALAHQRVRRQREWFL |
| | | HNESAHYAQSYTKIAIEDWSTKEMTSSEPRDAEEMKRVTRARNRSILDV |
| | | GWYELGRQIAYKSEATGAEFAKVDPGLRETETHVPEAIVRERDVDVSG |
| | | MLRGEAGISGTCSRCGGLLRASASGHADAECEVCLHVEVGDVNAAVNV |
| | | LKRAMFPGAAPPSKEKAKVTIGIKGRKKKRAA |
| Cas14 | 154 | MSRVELHRAYKFRLYPTPVQVAELSEWERQLRRLYNLGHEQRLLTLTR |
| ortholog 48 | | HLRPKSPGVLKGECLSCDSTQVQEVGADGRPKTTVRHAEQCPTLACRSC |
| | | GALRDAEGRTAHTVACAFVDYYRQGREMTELLAADDQLARVVCSARQ |
| | | EVLRDLDKAWQRWRKMPGFGKPRFKRRTDSCRIYFSTPKAWKLEGGHL |
| | | SFTGAATTVGAIKMRQDRNWPASVQFSSCHVVRDVDEWYAVFPLTFVA |
| | | EVARPKGGAVGINRGAVHAIADSTGRVVDSPRYYARALGVIRHRARLFD |
| | | RKVPSGHAVKPSPTKYRGLSAIEVDRVARATGFTPGRVVTEALNRGGVA |
| | | YAECALAAIAVLGHGPERPLTSDGRNREKARKFLALAHQRVRRQREWF |
| | | LHNESAHYARTYSKIAIEDWSTKEMTASEPQGEETRRVTRSRNRSILDVG |
| | | WYELGRQLAYKTEATGAEFAQVDPGLKETETNVPKAIADARDVDVSG |
| | | MLRGEAGISGTCSKCGGLLRAPASGHADAECEICLNVEVGDVNAAVNV |
| | | LKRAMFPGDAPPASGEKPKVSIGIKGRQKKKKAA |
| Cas14 | 155 | MEAIATGMSPERRVELGILPGSVELKRAYKFRLYPMKVQQAELSEWERQ |
| ortholog 49 | | LRRLYNLAHEQRLAALLRYRDWDFQKGACPSCRVAVPGVHTAACDHV |
| | | DYFRQAREMTQLLEVDAQLSRVICCARQEVLRDLDKAWQRWRKKLGG |
| | | RPRFKRRTDSCRIYLSTPKHWEIAGRYLRLSGLASSVGEIRIEQDRAFPEG |
| | | ALLSSCSIVRDVDEWYACLPLTFTQPIERAPHRSVGLNRGVVHALADSD |
| | | GRVVDSPKFFERALATVQKRSRDLARKVSGSRNAHKARIKLAKAHQRV |
| | | RRQRAAFLHQESAYYSKGFDLVALEDMSVRKMTATAGEAPEMGRGAQ |
| | | RDLNRGILDVGWYELARQIDYKRLAHGGELLRVDPGQTTPLACVTEEQP |
| | | ARGISSACAVCGIPLARPASGNARMRCTACGSSQVGDVNAAENVLTRAL |
| | | SSAPSGPKSPKASIKIKGRQKRLGTPANRAGEASGGDPPVRGPVEGGTLA |
| | | YVVEPVSESQSDT |
| Cas14 | 156 | MTVRTYKYRAYPTPEQAEALTSWLRFASQLYNAALEHRKNAWGRHDA |
| ortholog 50 | | HGRGFRFWDGDAAPRKKSDPPGRWVYRGGGGAHISKNDQGKLLTEFR |
| | | REHAELLPPGMPALVQHEVLARLERSMAAFFQRATKGQKAGYPRWRSE |
| | | HRYDSLTFGLTSPSKERFDPETGESLGRGKTVGAGTYHNGDLRLTGLGE |
| | | LRILEHRRIPMGAIPKSVIVRRSGKRWFVSIAMEMPSVEPAASGRPAVGL |
| | | DMGVVTWGTAFTADTSAAAALVADLRRMATDPSDCRRLEELEREAAQ |
| | | LSEVLAHCRARGLDPARPRRCPKELTKLYRRSLHRLGELDRACARIRRR |
| | | LQAAHDIAEPVPDEAGSAVLIEGSNAGMRHARRVARTQRRVARRTRAG |
| | | HAHSNRRKKAVQAYARAKERERSARGDHRHKVSRALVRQFEEISVEAL |
| | | DIKQLTVAPEHNPDPQPDLPAHVQRRRNRGELDAAWGAFFAALDYKAA |
| | | DAGGRVARKPAPHTTQECARCGTLVPKPISLRVHRCPACGYTAPRTVNS |
| | | ARNVLQRPLEEPGRAGPSGANGRGVPHAVA |
| Cas14 | 157 | MNCRYRYRIYPTPGQRQSLARLFGCVRVVWNDALFLCRQSEKLPKNSEL |
| ortholog 51 | | QKLCITQAKKTEARGWLGQVSAIPLQQSVADLGVAFKNFFQSRSGKRKG |
| | | KKVNPPRVKRRNNRQGARFTRGGFKVKTSKVYLARIGDIKIKWSRPLPS |
| | | EPSSVTVIKDCAGQYFLSFVVEVKPEIKPPKNPSIGIDLGLKTFASCSNGE |
| | | KIDSPDYSRLYRKLKRCQRRLAKRQRGSKRRERMRVKVAKLNAQIRDK |
| | | RKDFLHKLSTKVVNENQVIALEDLNVGGMLKNRKLSRAISQAGWYEFR |
| | | SLCEGKAEKHNRDFRVISRWEPTSQVCSECGYRWGKIDLSVRSIVCINCG |
| | | VEHDRDDNASVNIEQAGLKVGVGHTHDSKRTGSACKTSNGAVCVEPST |
| | | HREYVQLTLFDW |
| Cas14 | 158 | MKSRWTFRCYPTPEQEQHLARTFGCVRFVWNWALRARTDAFRAGERIG |
| ortholog 52 | | YPATDKALTLLKQQPETVWLNEVSSVCLQQALRDLQVAFSNFFDKRAA |
| | | HPSFKRKEARQSANYTERGFSFDHERRILKLAKIGAIKVKWSRKAIPHPSS |
| | | IRLIRTASGKYFVSLVVETQPAPMPETGESVGVDFGVARLATLSNGERIS |
| | | NPKHGAKWQRRLAFYQKRLARATKGSKRRMRIKRHVARIHEKIGNSRS |
| | | DTLHKLSTDLVTRFDLICVEDLNLRGMVKNHSLARSLHDASIGSAIRMIE |
| | | EKAERYGKNVVKIDRWFPSSKTCSDCGHIVEQLPLNVREWTCPECGTTH |
| | | DRDANAAANILAVGQTVSAHGGTVRRSRAKASERKSQRSANRQGVNR |
| | | A |
| Cas14 | 159 | KEPLNIGKTAKAVFKEIDPTSLNRAANYDASIELNCKECKFKPFKNVKRY |
| ortholog 53 | | EFNFYNNWYRCNPNSCLQSTYKAQVRKVEIGYEKLKNEILTQMQYYPW |
| | | FGRLYQNFFHDERDKMTSLDEIQVIGVQNKVFFNTVEKAWREIIKKRFK |
| | | DNKETMETIPELKHAAGHGKRKLSNKSLLRRRFAFVQKSFKFVDNSDVS |
| | | YRSFSNNIACVLPSRIGVDLGGVISRNPKREYIPQEISFNAFWKQHEGLKK |
| | | GRNIEIQSVQYKGETVKRIEADTGEDKAWGKNRQRRFTSLILKLVPKQG |
| | | GKKVWKYPEKRNEGNYEYFPIPIEFILDSGETSIRFGGDEGEAGKQKHLV |
| | | IPFNDSKATPLASQQTLLENSRFNAEVKSCIGLAIYANYFYGYARNYVISS |
| | | IYHKNSKNGQAITAIYLESIAHNYVKAIERQLQNLLLNLRDFSFMESHKK |
| | | ELKKYFGGDLEGTGGAQKRREKEEKIEKEIEQSYLPRLIRLSLTKMVTKQ |
| | | VEM |
| Cas14 | 160 | ELIVNENKDPLNIGKTAKAVFKEIDPTSINRAANYDASIELACKECKFKPF |
| ortholog 54 | | NNTKRHDFSFYSNWHRCSPNSCLQSTYRAKIRKTEIGYEKLKNEILNQM |
| | | QYYPWFGRLYQNFFNDQRDKMTSLDEIQVTGVQNKIFFNTVEKAWREII |
| | | KKRFRDNKETMRTIPDLKNKSGHGSRKLSNKSLLRRRFAFAQKSFKLVD |
| | | NSDVSYRAFSNNVACVLPSKIGVDIGGIINKDLKREYIPQEITFNVFWKQH |
| | | DGLKKGRNIEIHSVQYKGEIVKRIEADTGEDKAWGKNRQRRFTSLILKIT |
| | | PKQGGKKIWKFPEKKNASDYEYFPIPIEFILDNGDASIKFGGEEGEVGKQ |
| | | KHLLIPFNDSKATPLSSKQMLLETSRFNAEVKSTIGLALYANYFVSYARN |
| | | YVIKSTYHKNSKKGQIVTEIYLESISQNFVRAIQRQLQSLMLNLKDWGFM |
| | | QTHKKELKKYFGSDLEGSKGGQKRREKEEKIEKEIEASYLPRLIRLSLTKS |
| | | VTKAEEM |
| Cas14 | 161 | PEEKTSKLKPNSINLAANYDANEKFNCKECKFHPFKNKKRYEFNFYNNL |
| ortholog 55 | | HGCKSCTKSTNNPAVKRIEIGYQKLKFEIKNQMEAYPWFGRLRINFYSDE |
| | | KRKMSELNEMQVTGVKNKIFFDAIECAWREILKKRFRESKETLITIPKLK |
| | | NKAGHGARKHRNKKLLIRRRAFMKKNFHFLDNDSISYRSFANNIACVLP |
| | | SKVGVDIGGIISPDVGKDIKPVDISLNLMWASKEGIKSGRKVEIYSTQYD |
| | | GNMVKKIEAETGEDKSWGKNRKRRQTSLLLSIPKPSKQVQEFDFKEWPR |
| | | YKDIEKKVQWRGFPIKIIFDSNHNSIEFGTYQGGKQKVLPIPFNDSKTTPL |
| | | GSKMNKLEKLRFNSKIKSRLGSAIAANKFLEAARTYCVDSLYHEVSSAN |
| | | AIGKGKIFIEYYLEILSQNYIEAAQKQLQRFIESIEQWFVADPFQGRLKQY |
| | | FKDDLKRAKCFLCANREVQTTCYAAVKLHKSCAEKVKDKNKELAIKER |
| | | NNKEDAVIKEVEASNYPRVIRLKLTKTITNKAM |
| Cas14 | 162 | SESENKIIEQYYAFLYSFRDKYEKPEFKNRGDIKRKLQNKWEDFLKEQNL |
| ortholog 56 | | KNDKKLSNYIFSNRNFRRSYDREEENEEGIDEKKSKPKRINCFEKEKNLK |
| | | DQYDKDAINASANKDGAQKWGCFECIFFPMYKIESGDPNKRIIINKTRFK |
| | | LFDFYLNLKGCKSCLRSTYHPYRSNVYIESNYDKLKREIGNFLQQKNIFQ |
| | | RMRKAKVSEGKYLTNLDEYRLSCVAMHFKNRWLFFDSIQKVLRETIKQ |
| | | RLKQMRESYDEQAKTKRSKGHGRAKYEDQVRMIRRRAYSAQAHKLLD |
| | | NGYITLFDYDDKEINKVCLTAINQEGFDIGGYLNSDIDNVMPPIEISFHLK |
| | | WKYNEPILNIESPFSKAKISDYLRKIREDLNLERGKEGKARSKKNVRRKV |
| | | LASKGEDGYKKIFTDFFSKWKEELEGNAMERVLSQSSGDIQWSKKKRIH |
| | | YTTLVLNINLLDKKGVGNLKYYEIAEKTKILSFDKNENKFWPITIQVLLD |
| | | GYEIGTEYDEIKQLNEKTSKQFTIYDPNTKIIKIPFTDSKAVPLGMLGINIA |
| | | TLKTVKKTERDIKVSKIFKGGLNSKIVSKIGKGIYAGYFPTVDKEILEEVE |
| | | EDTLDNEFSSKSQRNIFLKSIIKNYDKMLKEQLFDFYSFLVRNDLGVRFLT |
| | | DRELQNIEDESFNLEKRFFETDRDRIARWFDNTNTDDGKEKFKKLANEIV |
| | | DSYKPRLIRLPVVRVIKRIQPVKQREM |
| Cas14 | 163 | KYSTRDFSELNEIQVTACKQDEFFKVIQNAWREIIKKRFLENRENFIEKKI |
| ortholog 57 | | FKNKKGRGKRQESDKTIQRNRASVMKNFQLIENEKIILRAPSGHVACVFP |
| | | VKVGLDIGGFKTDDLEKNIFPPRTITINVFWKNRDRQRKGRKLEVWGIK |
| | | ARTKLIEKVHKWDKLEEVKKKRLKSLEQKQEKSLDNWSEVNNDSFYKV |
| | | QIDELQEKIDKSLKGRTMNKILDNKAKESKEAEGLYIEWEKDFEGEMLR |
| | | RIEASTGGEEKWGKRRQRRHTSLLLDIKNNSRGSKEIINFYSYAKQGKKE |
| | | KKIEFFPFPLTITLDAEEESPLNIKSIPIEDKNATSKYFSIPFTETRATPLSILG |
| | | DRVQKFKTKNISGAIKRNLGSSISSCKIVQNAETSAKSILSLPNVKEDNNM |
| | | EIFINTMSKNYFRAMMKQMESFIFEMEPKTLIDPYKEKAIKWFEVAASSR |
| | | AKRKLKKLSKADIKKSELLLSNTEEFEKEKQEKLEALEKEIEEFYLPRIVR |
| | | LQLTKTILETPVM |
| Cas14 | 164 | KKLQLLGHKILLKEYDPNAVNAAANFETSTAELCGQCKMKPFKNKRRF |
| ortholog 58 | | QYTFGKNYHGCLSCIQNVYYAKKRIVQIAKEELKHQLTDSIASIPYKYTS |
| | | LFSNTNSIDELYILKQERAAFFSNTNSIDELYITGIENNIAFKVISAIWDEIIK |
| | | KRRQRYAESLTDTGTVKANRGHGGTAYKSNTRQEKIRALQKQTLHMVT |
| | | NPYISLARYKNNYIVATLPRTIGMHIGAIKDRDPQKKLSDYAINFNVFWS |
| | | DDRQLIELSTVQYTGDMVRKIEAETGENNKWGENMKRTKTSLLLEILTK |
| | | KTTDELTFKDWAFSTKKEIDSVTKKTYQGFPIGIIFEGNESSVKFGSQNYF |
| | | PLPFDAKITPPTAEGFRLDWLRKGSFSSQMKTSYGLAIYSNKVTNAIPAY |
| | | VIKNMFYKIARAENGKQIKAKFLKKYLDIAGNNYVPFIIMQHYRVLDTFE |
| | | EMPISQPKVIRLSLTKTQHIIIKKDKTDSKM |
| Cas14 | 165 | NTSNLINLGKKAINISANYDANLEVGCKNCKFLSSNGNFPRQTNVKEGC |
| ortholog 59 | | HSCEKSTYEPSIYLVKIGERKAKYDVLDSLKKFTFQSLKYQSKKSMKSRN |
| | | KKPKELKEFVIFANKNKAFDVIQKSYNHLILQIKKEINRMNSKKRKKNH |
| | | KRRLFRDREKQLNKLRLIESSNLFLPRENKGNNHVFTYVAIHSVGRDIGV |
| | | IGSYDEKLNFETELTYQLYFNDDKRLLYAYKPKQNKIIKIKEKLWNLRKE |
| | | KEPLDLEYEKPLNKSITFSIKNDNLFKVSKDLMLRRAKFNIQGKEKLSKE |
| | | ERKINRDLIKIKGLVNSMSYGRFDELKKEKNIWSPHIYREVRQKEIKPCLI |
| | | KNGDRIEIFEQLKKKMERLRRFREKRQKKISKDLIFAERIAYNFHTKSIKN |
| | | TSNKINIDQEAKRGKASYMRKRIGYETFKNKYCEQCLSKGNVYRNVQK |
| | | GCSCFENPFDWIKKGDENLLPKKNEDLRVKGAFRDEALEKQIVKIAFNIA |
| | | KGYEDFYDNLGESTEKDLKLKFKVGTTINEQESLKL |
| Cas14 | 166 | TSNPIKLGKKAINISANYDSNLQIGCKNCKFLSYNGNFPRQTNVKEGCHS |
| ortholog 60 | | CEKSTYEPPVYTVRIGERRSKYDVLDSLKKFIFLSLKYRQSKKMKTRSKG |
| | | IRGLEEFVISANLKKAMDVIQKSYRHLILNIKNEIVRMNGKKRNKNHKRL |
| | | LFRDREKQLNKLRLIEGSSFFKPPTVKGDNSIFTCVAIHNIGRDIGIAGDYF |
| | | DKLEPKIELTYQLYYEYNPKKESEINKRLLYAYKPKQNKIIEIKEKLWNL |
| | | RKEKSPLDLEYEKPLTKSITFLVKRDGVFRISKDLMLRKAKFIIQGKEKLS |
| | | KEERKINRDLIKIKSNIISLTYGRFDELKKDKTIWSPHIFRDVKQGKITPCIE |
| | | RKGDRMDIFQQLRKKSERLRENRKKRQKKISKDLIFAERIAYNFHTKSIK |
| | | NTSNLINIKHEAKRGKASYMRKRIGNETFRIKYCEQCFPKNNVYKNVQK |
| | | GCSCFEDPFEYIKKGNEDLIPNKNQDLKAKGAFRDDALEKQIIKVAFNIA |
| | | KGYEDFYENLKKTTEKDIRLKFKVGTIISEEM |
| Cas14 | 167 | NNSINLSKKAINISANYDANLQVRCKNCKFLSSNGNFPRQTDVKEGCHS |
| ortholog 61 | | CEKSTYEPPVYDVKIGEIKAKYEVLDSLKKFTFQSLKYQLSKSMKFRSKK |
| | | IKELKEFVIFAKESKALNVINRSYKHLILNIKNDINRMNSKKRIKNHKGRL |
| | | FLDRQKQLSKLKLIEGSSFFVPAKNVGNKSVFTCVAIHSIGRDIGIAGLYD |
| | | SFTKPVNEITYQIFFSGERRLLYAYKPKQLKILSIKENLWSLKNEKKPLDL |
| | | LYEKPLGKNLNFNVKGGDLFRVSKDLMIRNAKFNVHGRQRLSDEERLIN |
| | | RNFIKIKGEVVSLSYGRFEELKKDRKLWSPHIFKDVRQNKIKPCLVMQG |
| | | QRIDIFEQLKRKLELLKKIRKSRQKKLSKDLIFGERIAYNFHTKSIKNTSN |
| | | KINIDSDAKRGRASYMRKRIGNETFKLKYCDVCFPKANVYRRVQNGCSC |
| | | SENPYNYIKKGDKDLLPKKDEGLAIKGAFRDEKLNKQIIKVAFNIAKGYE |
| | | DFYDDLKKRTEKDVDLKFKIGTTVLDQKPMEIFDGIVITWL |
| Cas14 | 168 | LLTTVVETNNLAKKAINVAANFDANIDRQYYRCTPNLCRFIAQSPRETKE |
| ortholog 62 | | KDAGCSSCTQSTYDPKVYVIKIGKLLAKYEILKSLKRFLFMNRYFKQKK |
| | | TERAQQKQKIGTELNEMSIFAKATNAMEVIKRATKHCTYDIIPETKSLQM |
| | | LKRRRHRVKVRSLLKILKERRMKIKKIPNTFIEIPKQAKKNKSDYYVAAA |
| | | LKSCGIDVGLCGAYEKNAEVEAEYTYQLYYEYKGNSSTKRILYCYNNPQ |
| | | KNIREFWEAFYIQGSKSHVNTPGTIRLKMEKFLSPITIESEALDFRVWNSD |
| | | LKIRNGQYGFIKKRSLGKEAREIKKGMGDIKRKIGNLTYGKSPSELKSIH |
| | | VYRTERENPKKPRAARKKEDNFMEIFEMQRKKDYEVNKKRRKEATDA |
| | | AKIMDFAEEPIRHYHTNNLKAVRRIDMNEQVERKKTSVFLKRIMQNGYR |
| | | GNYCRKCIKAPEGSNRDENVLEKNEGCLDCIGSEFIWKKSSKEKKGLWH |
| | | TNRLLRRIRLQCFTTAKAYENFYNDLFEKKESSLDIIKLKVSITTKSM |
| Cas14 | 169 | ASTMNLAKQAINFAANYDSNLEIGCKGCKFMSTWSKKSNPKFYPRQNN |
| ortholog 63 | | QANKCHSCTYSTGEPEVPIIEIGERAAKYKIFTALKKFVFMSVAYKERRR |
| | | QRFKSKKPKELKELAICSNREKAMEVIQKSVVHCYGDVKQEIPRIRKIKV |
| | | LKNHKGRLFYKQKRSKIKIAKLEKGSFFKTFIPKVHNNGCHSCHEASLNK |
| | | PILVTTALNTIGADIGLINDYSTIAPTETDISWQVYYEFIPNGDSEAVKKRL |
| | | LYFYKPKGALIKSIRDKYFKKGHENAVNTGFFKYQGKIVKGPIKFVNNEL |
| | | DFARKPDLKSMKIKRAGFAIPSAKRLSKEDREINRESIKIKNKIYSLSYGR |
| | | KKTLSDKDIIKHLYRPVRQKGVKPLEYRKAPDGFLEFFYSLKRKERRLRK |
| | | QKEKRQKDMSEIIDAADEFAWHRHTGSIKKTTNHINFKSEVKRGKVPIM |
| | | KKRIANDSFNTRHCGKCVKQGNAINKYYIEKQKNCFDCNSIEFKWEKAA |
| | | LEKKGAFKLNKRLQYIVKACFNVAKAYESFYEDFRKGEEESLDLKFKIG |
| | | TTTTLKQYPQNKARAM |
| Cas14 | 170 | HSHNLMLTKLGKQAINFAANYDANLEIGCKNCKFLSYSPKQANPKKYPR |
| ortholog 64 | | QTDVHEDGNIACHSCMQSTKEPPVYIVPIGERKSKYEILTSLNKFTFLALK |
| | | YKEKKRQAFRAKKPKELQELAIAFNKEKAIKVIDKSIQHLILNIKPEIARIQ |
| | | RQKRLKNRKGKLLYLHKRYAIKMGLIKNGKYFKVGSPKKDGKKLLVLC |
| | | ALNTIGRDIGIIGNIEENNRSETEITYQLYFDCLDANPNELRIKEIEYNRLK |
| | | SYERKIKRLVYAYKPKQTKILEIRSKFFSKGHENKVNTGSFNFENPLNKSI |
| | | SIKVKNSAFDFKIGAPFIMLRNGKFHIPTKKRLSKEEREINRTLSKIKGRVF |
| | | RLTYGRNISEQGSKSLHIYRKERQHPKLSLEIRKQPDSFIDEFEKLRLKQN |
| | | FISKLKKQRQKKLADLLQFADRIAYNYHTSSLEKTSNFINYKPEVKRGRT |
| | | SYIKKRIGNEGFEKLYCETCIKSNDKENAYAVEKEELCFVCKAKPFTWK |
| | | KTNKDKLGIFKYPSRIKDFIRAAFTVAKSYNDFYENLKKKDLKNEIFLKF |
| | | KIGLILSHEKKNHISIAKSVAEDERISGKSIKNILNKSIKLEKNCYSCFFHKE |
| | | DM |
| Cas14 | 171 | SLERVIDKRNLAKKAINIAANFDANINKGFYRCETNQCMFIAQKPRKTNN |
| ortholog 65 | | TGCSSCLQSTYDPVIYVVKVGEMLAKYEILKSLKRFVFMNRSFKQKKTE |
| | | KAKQKERIGGELNEMSIFANAALAMGVIKRAIRHCHVDIRPEINRLSELK |
| | | KTKHRVAAKSLVKIVKQRKTKWKGIPNSFIQIPQKARNKDADFYVASAL |
| | | KSGGIDIGLCGTYDKKPHADPRWTYQLYFDTEDESEKRLLYCYNDPQAK |
| | | IRDFWKTFYERGNPSMVNSPGTIEFRMEGFFEKMTPISIESKDFDFRVWN |
| | | KDLLIRRGLYEIKKRKNLNRKAREIKKAMGSVKRVLANMTYGKSPTDK |
| | | KSIPVYRVEREKPKKPRAVRKEENELADKLENYRREDFLIRNRRKREATE |
| | | IAKIIDAAEPPIRHYHTNHLRAVKRIDLSKPVARKNTSVFLKRIMQNGYR |
| | | GNYCKKCIKGNIDPNKDECRLEDIKKCICCEGTQNIWAKKEKLYTGRINV |
| | | LNKRIKQMKLECFNVAKAYENFYDNLAALKEGDLKVLKLKVSIPALNPE |
| | | ASDPEEDM |
| Cas14 | 172 | NASINLGKRAINLSANYDSNLVIGCKNCKFLSFNGNFPRQTNVREGCHSC |
| ortholog 66 | | DKSTYAPEVYIVKIGERKAKYDVLDSLKKFTFQSLKYQIKKSMRERSKK |
| | | PKELLEFVIFANKDKAFNVIQKSYEHLILNIKQEINRMNGKKRIKNHKKR |
| | | LFKDREKQLNKLRLIGSSSLFFPRENKGDKDLFTYVAIHSVGRDIGVAGS |
| | | YESHIEPISDLTYQLFINNEKRLLYAYKPKQNKIIELKENLWNLKKEKKPL |
| | | DLEFTKPLEKSITFSVKNDKLFKVSKDLMLRQAKFNIQGKEKLSKEERQI |
| | | NRDFSKIKSNVISLSYGRFEELKKEKNIWSPHIYREVKQKEIKPCIVRKGD |
| | | RIELFEQLKRKMDKLKKFRKERQKKISKDLNFAERIAYNFHTKSIKNTSN |
| | | KINIDQEAKRGKASYMRKRIGNESFRKKYCEQCFSVGNVYHNVQNGCS |
| | | CFDNPIELIKKGDEGLIPKGKEDRKYKGALRDDNLQMQIIRVAFNIAKGY |
| | | EDFYNNLKEKTEKDLKLKFKIGTTISTQESNNKEM |
| Cas14 | 173 | SNLIKLGKQAINFAANYDANLEVGCKNCKFLSSTNKYPRQTNVHLDNK |
| ortholog 67 | | MACRSCNQSTMEPAIYIVRIGEKKAKYDIYNSLTKFNFQSLKYKAKRSQ |
| | | RFKPKQPKELQELSIAVRKEKALDIIQKSIDHLIQDIRPEIPRIKQQKRYKN |
| | | HVGKLFYLQKRRKNKLNLIGKGSFFKVFSPKEKKNELLVICALTNIGRDI |
| | | GLIGNYNTIINPLFEVTYQLYYDYIPKKNNKNVQRRLLYAYKSKNEKILK |
| | | LKEAFFKRGHENAVNLGSFSYEKPLEKSLTLKIKNDKDDFQVSPSLRIRT |
| | | GRFFVPSKRNLSRQEREINRRLVKIKSKIKNMTYGKFETARDKQSVHIFR |
| | | LERQKEKLPLQFRKDEKEFMEEFQKLKRRTNSLKKLRKSRQKKLADLLQ |
| | | LSEKVVYNNHTGTLKKTSNFLNFSSSVKRGKTAYIKELLGQEGFETLYCS |
| | | NCINKGQKTRYNIETKEKCFSCKDVPFVWKKKSTDKDRKGAFLFPAKLK |
| | | DVIKATFTVAKAYEDFYDNLKSIDEKKPYIKFKIGLILAHVRHEHKARAK |
| | | EEAGQKNIYNKPIKIDKNCKECFFFKEEAM |
| Cas14 | 174 | NTTRKKFRKRTGFPQSDNIKLAYCSAIVRAANLDADIQKKHNQCNPNLC |
| ortholog 68 | | VGIKSNEQSRKYEHSDRQALLCYACNQSTGAPKVDYIQIGEIGAKYKILQ |
| | | MVNAYDFLSLAYNLTKLRNGKSRGHQRMSQLDEVVIVADYEKATEVIK |
| | | RSINHLLDDIRGQLSKLKKRTQNEHITEHKQSKIRRKLRKLSRLLKRRRW |
| | | KWGTIPNPYLKNWVFTKKDPELVTVALLHKLGRDIGLVNRSKRRSKQK |
| | | LLPKVGFQLYYKWESPSLNNIKKSKAKKLPKRLLIPYKNVKLFDNKQKL |
| | | ENAIKSLLESYQKTIKVEFDQFFQNRTEEIIAEEQQTLERGLLKQLEKKKN |
| | | EFASQKKALKEEKKKIKEPRKAKLLMEESRSLGFLMANVSYALFNTTIE |
| | | DLYKKSNVVSGCIPQEPVVVFPADIQNKGSLAKILFAPKDGFRIKFSGQH |
| | | LTIRTAKFKIRGKEIKILTKTKREILKNIEKLRRVWYREQHYKLKLFGKEV |
| | | SAKPRFLDKRKTSIERRDPNKLADQTDDRQAELRNKEYELRHKQHKMA |
| | | ERLDNIDTNAQNLQTLSFWVGEADKPPKLDEKDARGFGVRTCISAWKW |
| | | FMEDLLKKQEEDPLLKLKLSIM |
| Cas14 | 175 | PKKPKFQKRTGFPQPDNLRKEYCLAIVRAANLDADFEKKCTKCEGIKTN |
| ortholog 69 | | KKGNIVKGRTYNSADKDNLLCYACNISTGAPAVDYVFVGALEAKYKIL |
| | | QMVKAYDFHSLAYNLAKLWKGRGRGHQRMGGLNEVVIVSNNEKALD |
| | | VIEKSLNHFHDEIRGELSRLKAKFQNEHLHVHKESKLRRKLRKISRLLKR |
| | | RRWKWDVIPNSYLRNFTFTKTRPDFISVALLHRVGRDIGLVTKTKIPKPT |
| | | DLLPQFGFQIYYTWDEPKLNKLKKSRLRSEPKRLLVPYKKIELYKNKSVL |
| | | EEAIRHLAEVYTEDLTICFKDFFETQKRKFVSKEKESLKRELLKELTKLK |
| | | KDFSERKTALKRDRKEIKEPKKAKLLMEESRSLGFLAANTSYALFNLIAA |
| | | DLYTKSKKACSTKLPRQLSTILPLEIKEHKSTTSLAIKPEEGFKIRFSNTHL |
| | | SIRTPKFKMKGADIKALTKRKREILKNATKLEKSWYGLKHYKLKLYGKE |
| | | VAAKPRFLDKRNPSIDRRDPKELMEQIENRRNEVKDLEYEIRKGQHQMA |
| | | KRLDNVDTNAQNLQTKSFWVGEADKPPELDSMEAKKLGLRTCISAWK |
| | | WFMKDLVLLQEKSPNLKLKLSLTEM |
| Cas14 | 176 | KFSKRQEGFLIPDNIDLYKCLAIVRSANLDADVQGHKSCYGVKKNGTYR |
| ortholog 70 | | VKQNGKKGVKEKGRKYVFDLIAFKGNIEKIPHEAIEEKDQGRVIVLGKF |
| | | NYKLILNIEKNHNDRASLEIKNKIKKLVQISSLETGEFLSDLLSGKIGIDEV |
| | | YGIIEPDVFSGKELVCKACQQSTYAPLVEYMPVGELDAKYKILSAIKGYD |
| | | FLSLAYNLSRNRANKKRGHQKLGGGELSEVVISANYDKALNVIKRSINH |
| | | YHVEIKPEISKLKKKMQNEPLKVMKQARIRRELHQLSRKVKRLKWKWG |
| | | MIPNPELQNIIFEKKEKDFVSYALLHTLGRDIGLFKDTSMLQVPNISDYGF |
| | | QIYYSWEDPKLNSIKKIKDLPKRLLIPYKRLDFYIDTILVAKVIKNLIELYR |
| | | KSYVYETFGEEYGYAKKAEDILFDWDSINLSEGIEQKIQKIKDEFSDLLYE |
| | | ARESKRQNFVESFENILGLYDKNFASDRNSYQEKIQSMIIKKQQENIEQK |
| | | LKREFKEVIERGFEGMDQNKKYYKVLSPNIKGGLLYTDTNNLGFFRSHL |
| | | AFMLLSKISDDLYRKNNLVSKGGNKGILDQTPETMLTLEFGKSNLPNISI |
| | | KRKFFNIKYNSSWIGIRKPKFSIKGAVIREITKKVRDEQRLIKSLEGVWHK |
| | | STHFKRWGKPRFNLPRHPDREKNNDDNLMESITSRREQIQLLLREKQKQ |
| | | QEKMAGRLDKIDKEIQNLQTANFQIKQIDKKPALTEKSEGKQSVRNALS |
| | | AWKWFMEDLIKYQKRTPILQLKLAKM |
| Cas14 | 177 | KFSKRQEGFVIPENIGLYKCLAIVRSANLDADVQGHVSCYGVKKNGTYV |
| ortholog 71 | | LKQNGKKSIREKGRKYASDLVAFKGDIEKIPFEVIEEKKKEQSIVLGKFN |
| | | YKLVLDVMKGEKDRASLTMKNKSKKLVQVSSLGTDEFLLTLLNEKFGIE |
| | | EIYGIIEPEVFSGKKLVCKACQQSTYAPLVEYMPVGELDSKYKILSAIKGY |
| | | DFLSLAYNLARHRSNKKRGHQKLGGGELSEVVISANNAKALNVIKRSLN |
| | | HYYSEIKPEISKLRKKMQNEPLKVGKQARMRRELHQLSRKVKRLKWKW |
| | | GKIPNLELQNITFKESDRDFISYALLHTLGRDIGMFNKTEIKMPSNILGYG |
| | | FQIYYDWEEPKLNTIKKSKNTPKRILIPYKKLDFYNDSILVARAIKELVGL |
| | | FQESYEWEIFGNEYNYAKEAEVELIKLDEESINGNVEKKLQRIKENFSNL |
| | | LEKAREKKRQNFIESFESIARLYDESFTADRNEYQREIQSFIIEKQKQSIEK |
| | | KLKNEFKKIVEKKFNEQEQGKKHYRVLNPTIINEFLPKDKNNLGFLRSKI |
| | | AFILLSKISDDLYKKSNAVSKGGEKGIIKQQPETILDLEFSKSKLPSINIKK |
| | | KLFNIKYTSSWLGIRKPKFNIKGAKIREITRRVRDVQRTLKSAESSWYAST |
| | | HFRRWGFPRFNQPRHPDKEKKSDDRLIESITLLREQIQILLREKQKGQKE |
| | | MAGRLDDVDKKIQNLQTANFQIKQTGDKPALTEKSAGKQSFRNALSAW |
| | | KWFMENLLKYQNKTPDLKLKIARTVM |
| Cas14 | 178 | KWIEPNNIDFNKCLAITRSANLDADVQGHKMCYGIKTNGTYKAIGKINK |
| ortholog 72 | | KHNTGIIEKRRTYVYDLIVTKEKNEKIVKKTDFMAIDEEIEFDEKKEKLL |
| | | KKYIKAEVLGTGELIRKDLNDGEKFDDLCSIEEPQAFRRSELVCKACNQS |
| | | TYASDIRYIPIGEIEAKYKILKAIKGYDFLSLKYNLGRLRDSKKRGHQKM |
| | | GQGELKEFVICANKEKALDVIKRSLNHYLNEVKDEISRLNKKMQNEPLK |
| | | VNDQARWRRELNQISRRLKRLKWKWGEIPNPELKNLIFKSSRPEFVSYA |
| | | LIHTLGRDIGLINETELKPNNIQEYGFQIYYKWEDPELNHIKKVKNIPKRFI |
| | | IPYKNLDLFGKYTILSRAIEGILKLYSSSFQYKSFKDPNLFAKEGEKKITNE |
| | | DFELGYDEKIKKIKDDFKSYKKALLEKKKNTLEDSLNSILSVYEQSLLTE |
| | | QINNVKKWKEGLLKSKESIHKQKKIENIEDIISRIEELKNVEGWIRTKERDI |
| | | VNKEETNLKREIKKELKDSYYEEVRKDFSDLKKGEESEKKPFREEPKPIVI |
| | | KDYIKFDVLPGENSALGFFLSHLSFNLFDSIQYELFEKSRLSSSKHPQIPETI |
| | | LDL |
| Cas14 | 179 | FRKFVKRSGAPQPDNLNKYKCIAIVRAANLDADIMSNESSNCVMCKGIK |
| ortholog 73 | | MNKRKTAKGAAKTTELGRVYAGQSGNLLCTACTKSTMGPLVDYVPIGR |
| | | IRAKYTILRAVKEYDFLSLAYNLARTRVSKKGGRQKMHSLSELVIAAEY |
| | | EIAWNIIKSSVIHYHQETKEEISGLRKKLQAEHIHKNKEARIRREMHQISR |
| | | RIKRLKWKWHMIPNSELHNFLFKQQDPSFVAVALLHTLGRDIGMINKPK |
| | | GSAKREFIPEYGFQIYYKWMNPKLNDINKQKYRKMPKRSLIPYKNLNVF |
| | | GDRELIENAMHKLLKLYDENLEVKGSKFFKTRVVAISSKESEKLKRDLL |
| | | WKGELAKIKKDFNADKNKMQELFKEVKEPKKANALMKQSRNMGFLLQ |
| | | NISYGALGLLANRMYEASAKQSKGDATKQPSIVIPLEMEFGNAFPKLLLR |
| | | SGKFAMNVSSPWLTIRKPKFVIKGNKIKNITKLMKDEKAKLKRLETSYH |
| | | RATHFRPTLRGSIDWDSPYFSSPKQPNTHRRSPDRLSADITEYRGRLKSVE |
| | | AELREGQRAMAKKLDSVDMTASNLQTSNFQLEKGEDPRLTEIDEKGRSI |
| | | RNCISSWKKFMEDLMKAQEANPVIKIKIALKDESSVLSEDSM |
| Cas14 | 180 | KFHPENLNKSYCLAIVRAANLDADIQGHINCIGIKSNKSDRNYENKLESL |
| ortholog 74 | | QNVELLCKACTKSTYKPNINSVPVGEKKAKYSILSEIKKYDFNSLVYNLK |
| | | KYRKGKSRGHQKLNELRELVITSEYKKALDVINKSVNHYLVNIKNKMS |
| | | KLKKILQNEHIHVGTLARIRRERNRISRKLDHYRKKWKFVPNKILKNYVF |
| | | KNQSPDFVSVALLHKLGRDIGLITKTAILQKSFPEYSLQLYYKYDTPKLN |
| | | YLKKSKFKSLPKRILISYKYPKFDINSNYIEESIDKLLKLYEESPIYKNNSKI |
| | | IEFFKKSEDNLIKSENDSLKRGIMKEFEKVTKNFSSKKKKLKEELKLKNE |
| | | DKNSKMLAKVSRPIGFLKAYLSYMLFNIISNRIFEFSRKSSGRIPQLPSCIIN |
| | | LGNQFENFKNELQDSNIGSKKNYKYFCNLLLKSSGFNISYEEEHLSIKTPN |
| | | FFINGRKLKEITSEKKKIRKENEQLIKQWKKLTFFKPSNLNGKKTSDKIRF |
| | | KSPNNPDIERKSEDNIVENIAKVKYKLEDLLSEQRKEFNKLAKKHDGVD |
| | | VEAQCLQTKSFWIDSNSPIKKSLEKKNEKVSVKKKMKAIRSCISAWKWF |
| | | MADLIEAQKETPMIKLKLALM |
| Cas14 | 181 | TTLVPSHLAGIEVMDETTSRNEDMIQKETSRSNEDENYLGVKNKCGINV |
| ortholog 75 | | HKSGRGSSKHEPNMPPEKSGEGQMPKQDSTEMQQRFDESVTGETQVSA |
| | | GATASIKTDARANSGPRVGTARALIVKASNLDRDIKLGCKPCEYIRSELP |
| | | MGKKNGCNHCEKSSDIASVPKVESGFRKAKYELVRRFESFAADSISRHL |
| | | GKEQARTRGKRGKKDKKEQMGKVNLDEIAILKNESLIEYTENQILDARS |
| | | NRIKEWLRSLRLRLRTRNKGLKKSKSIRRQLITLRRDYRKWIKPNPYRPD |
| | | EDPNENSLRLHTKLGVDIGVQGGDNKRMNSDDYETSFSITWRDTATRKI |
| | | CFTKPKGLLPRHMKFKLRGYPELILYNEELRIQDSQKFPLVDWERIPIFKL |
| | | RGVSLGKKKVKALNRITEAPRLVVAKRIQVNIESKKKKVLTRYVYNDKS |
| | | INGRLVKAEDSNKDPLLEFKKQAEEINSDAKYYENQEIAKNYLWGCEGL |
| | | HKNLLEEQTKNPYLAFKYGFLNIV |
| Cas14 | 182 | LDFKRTCSQELVLLPEIEGLKLSGTQGVTSLAKKLINKAANVDRDESYGC |
| ortholog 76 | | HHCIHTRTSLSKPVKKDCNSCNQSTNHPAVPITLKGYKIAFYELWHRFTS |
| | | WAVDSISKALHRNKVMGKVNLDEYAVVDNSHIVCYAVRKCYEKRQRS |
| | | VRLHKRAYRCRAKHYNKSQPKVGRIYKKSKRRNARNLKKEAKRYFQP |
| | | NEITNGSSDALFYKIGVDLGIAKGTPETEVKVDVSICFQVYYGDARRVLR |
| | | VRKMDELQSFHLDYTGKLKLKGIGNKDTFTIAKRNESLKWGSTKYEVSR |
| | | AHKKFKPFGKKGSVKRKCNDYFRSIASWSCEAASQRAQSNLKNAFPYQ |
| | | KALVKCYKNLDYKGVKKNDMWYRLCSNRIFRYSRIAEDIAQYQSDKGK |
| | | AKFEFVILAQSVAEYDISAIM |
| Cas14 | 183 | VFLTDDKRKTALRKIRSAFRKTAEIALVRAQEADSLDRQAKKLTIETVSF |
| ortholog 77 | | GAPGAKNAFIGSLQGYNWNSHRANVPSSGSAKDVFRITELGLGIPQSAH |
| | | EASIGKSFELVGNVVRYTANLLSKGYKKGAVNKGAKQQREIKGKEQLSF |
| | | DLISNGPISGDKLINGQKDALAWWLIDKMGFHIGLAMEPLSSPNTYGITL |
| | | QAFWKRHTAPRRYSRGVIRQWQLPFGRQLAPLIHNFFRKKGASIPIVLTN |
| | | ASKKLAGKGVLLEQTALVDPKKWWQVKEQVTGPLSNIWERSVPLVLYT |
| | | ATFTHKHGAAHKRPLTLKVIRISSGSVFLLPLSKVTPGKLVRAWMPDINI |
| | | LRDGRPDEAAYKGPDLIRARERSFPLAYTCVTQIADEWQKRALESNRDSI |
| | | TPLEAKLVTGSDLLQIHSTVQQAVEQGIGGRISSPIQELLAKDALQLVLQ |
| | | QLFMTVDLLRIQWQLKQEVADGNTSEKAVGWAIRISNIHKDAYKTAIEP |
| | | CTSALKQAWNPLSGFEERTFQLDASIVRKRSTAKTPDDELVIVLRQQAAE |
| | | MTVAVTQSVSKELMELAVRHSATLHLLVGEVASKQLSRSADKDRGAM |
| | | DHWKLLSQSM |
| Cas14 | 184 | EDLLQKALNTATNVAAIERHSCISCLFTESEIDVKYKTPDKIGQNTAGCQ |
| ortholog 78 | | SCTFRVGYSGNSHTLPMGNRIALDKLRETIQRYAWHSLLFNVPPAPTSKR |
| | | VRAISELRVAAGRERLFTVITFVQTNILSKLQKRYAANWTPKSQERLSRL |
| | | REEGQHILSLLESGSWQQKEVVREDQDLIVCSALTKPGLSIGAFCRPKYL |
| | | KPAKHALVLRLIFVEQWPGQIWGQSKRTRRMRRRKDVERVYDISVQAW |
| | | ALKGKETRISECIDTMRRHQQAYIGVLPFLILSGSTVRGKGDCPILKEITR |
| | | MRYCPNNEGLIPLGIFYRGSANKLLRVVKGSSFTLPMWQNIETLPHPEPF |
| | | SPEGWTATGALYEKNLAYWSALNEAVDWYTGQILSSGLQYPNQNEFLA |
| | | RLQNVIDSIPRKWFRPQGLKNLKPNGQEDIVPNEFVIPQNAIRAHHVIEW |
| | | YHKTNDLVAKTLLGWGSQTTLNQTRPQGDLRFTYTRYYFREKEVPEV |
| Cas14 | 185 | VPKKKLMRELAKKAVFEAIFNDPIPGSFGCKRCTLIDGARVTDAIEKKQG |
| ortholog 79 | | AKRCAGCEPCTFHTLYDSVKHALPAATGCDRTAIDTGLWEILTALRSYN |
| | | WMSFRRNAVSDASQKQVWSIEELAIWADKERALRVILSALTHTIGKLKN |
| | | GFSRDGVWKGGKQLYENLAQKDLAKGLFANGEIFGKELVEADHDMLA |
| | | WTIVPNHQFHIGLIRGNWKPAAVEASTAFDARWLTNGAPLRDTRTHGH |
| | | RGRRFNRTEKLTVLCIKRDGGVSEEFRQERDYELSVMLLQPKNKLKPEP |
| | | KGELNSFEDLHDHWWFLKGDEATALVGLTSDPTVGDFIQLGLYIRNPIK |
| | | AHGETKRRLLICFEPPIKLPLRRAFPSEAFKTWEPTINVFRNGRRDTEAYY |
| | | DIDRARVFEFPETRVSLEHLSKQWEVLRLEPDRENTDPYEAQQNEGAEL |
| | | QVYSLLQEAAQKMAPKVVIDPFGQFPLELFSTFVAQLFNAPLSDTKAKIG |
| | | KPLDSGFVVESHLHLLEEDFAYRDFVRVTFMGTEPTFRVIHYSNGEGYW |
| | | KKTVLKGKNNIRTALIPEGAKAAVDAYKNKRCPLTLEAAILNEEKDRRL |
| | | VLGNKALSLLAQTARGNLTILEALAAEVLRPLSGTEGVVHLHACVTRHS |
| | | TLTESTETDNM |
| Cas14 | 186 | VEKLFSERLKRAMWLKNEAGRAPPAETLTLKHKRVSGGHEKVKEELQR |
| ortholog 80 | | VLRSLSGTNQAAWNLGLSGGREPKSSDALKGEKSRVVLETVVFHSGHN |
| | | RVLYDVIEREDQVHQRSSIMHMRRKGSNLLRLWGRSGKVRRKMREEVA |
| | | EIKPVWHKDSRWLAIVEEGRQSVVGISSAGLAVFAVQESQCTTAEPKPLE |
| | | YVVSIWFRGSKALNPQDRYLEFKKLKTTEALRGQQYDPIPFSLKRGAGC |
| | | SLAIRGEGIKFGSRGPIKQFFGSDRSRPSHADYDGKRRLSLFSKYAGDLA |
| | | DLTEEQWNRTVSAFAEDEVRRATLANIQDFLSISHEKYAERLKKRIESIEE |
| | | PVSASKLEAYLSAIFETFVQQREALASNFLMRLVESVALLISLEEKSPRVE |
| | | FRVARYLAESKEGFNRKAM |
| Cas14 | 187 | VVITQSELYKERLLRVMEIKNDRGRKEPRESQGLVLRFTQVTGGQEKVK |
| ortholog 81 | | QKLWLIFEGFSGTNQASWNFGQPAGGRKPNSGDALKGPKSRVTYETVV |
| | | FHFGLRLLSAVIERHNLKQQRQTMAYMKRRAAARKKWARSGKKCSRM |
| | | RNEVEKIKPKWHKDPRWFDIVKEGEPSIVGISSAGFAIYIVEEPNFPRQDP |
| | | LEIEYAISIWFRRDRSQYLTFKKIQKAEKLKELQYNPIPFRLKQEKTSLVF |
| | | ESGDIKFGSRGSIEHFRDEARGKPPKADMDNNRRLTMFSVFSGNLTNLTE |
| | | EQYARPVSGLLAPDEKRMPTLLKKLQDFFTPIHEKYGERIKQRLANSEAS |
| | | KRPFKKLEEYLPAIYLEFRARREGLASNWVLVLINSVRTLVRIKSEDPYIE |
| | | FKVSQYLLEKEDNKAL |
| Cas14 | 188 | KQDALFEERLKKAIFIKRQADPLQREELSLLPPNRKIVTGGHESAKDTLK |
| ortholog 82 | | QILRAINGTNQASWNPGTPSGKRDSKSADALAGPKSRVKLETVVFHVGH |
| | | RLLKKVVEYQGHQKQQHGLKAFMRTCAAMRKKWKRSGKVVGELREQ |
| | | LANIQPKWHYDSRPLNLCFEGKPSVVGLRSAGIALYTIQKSVVPVKEPKP |
| | | IEYAVSIWFRGPKAMDREDRCLEFKKLKIATELRKLQFEPIVSTLTQGIKG |
| | | FSLYIQGNSVKFGSRGPIKYFSNESVRQRPPKADPDGNKRLALFSKFSGD |
| | | LSDLTEEQWNRPILAFEGIIRRATLGNIQDYLTVGHEQFAISLEQLLSEKES |
| | | VLQMSIEQQRLKKNLGKKAENEWVESFGAEQARKKAQGIREYISGFFQE |
| | | YCSQREQWAENWVQQLNKSVRLFLTIQDSTPFIEFRVARYLPKGEKKKG |
| | | KAM |
| Cas14 | 189 | ANHAERHKRLRKEANRAANRNRPLVADCDTGDPLVGICRLLRRGDKM |
| ortholog 83 | | QPNKTGCRSCEQVEPELRDAILVSGPGRLDNYKYELFQRGRAMAVHRLL |
| | | KRVPKLNRPKKAAGNDEKKAENKKSEIQKEKQKQRRMMPAVSMKQVS |
| | | VADFKHVIENTVRHLFGDRRDREIAECAALRAASKYFLKSRRVRPRKLP |
| | | KLANPDHGKELKGLRLREKRAKLKKEKEKQAELARSNQKGAVLHVAT |
| | | LKKDAPPMPYEKTQGRNDYTTFVISAAIKVGATRGTKPLLTPQPREWQC |
| | | SLYWRDGQRWIRGGLLGLQAGIVLGPKLNRELLEAVLQRPIECRMSGCG |
| | | NPLQVRGAAVDFFMTTNPFYVSGAAYAQKKFKPFGTKRASEDGAAAKA |
| | | REKLMTQLAKVLDKVVTQAAHSPLDGIWETRPEAKLRAMIMALEHEWI |
| | | FLRPGPCHNAAEEVIKCDCTGGHAILWALIDEARGALEHKEFYAVTRAH |
| | | THDCEKQKLGGRLAGFLDLLIAQDVPLDDAPAARKIKTLLEATPPAPCY |
| | | KAATSIATCDCEGKFDKLWAIIDATRAGHGTEDLWARTLAYPQNVNCK |
| | | CKAGKDLTHRLADFLGLLIKRDGPFRERPPHKVTGDRKLVFSGDKKCKG |
| | | HQYVILAKAHNEEVVRAWISRWGLKSRTNKAGYAATELNLLLNWLSIC |
| | | RRRWMDMLTVQRDTPYIRMKTGRLVVDDKKERKAM |
| Cas14 | 190 | AKQREALRVALERGIVRASNRTYTLVTNCTKGGPLPEQCRMIERGKARA |
| ortholog 84 | | MKWEPKLVGCGSCAAATVDLPAIEEYAQPGRLDVAKYKLTTQILAMAT |
| | | RRMMVRAAKLSRRKGQWPAKVQEEKEEPPEPKKMLKAVEMRPVAIVD |
| | | FNRVIQTTIEHLWAERANADEAELKALKAAAAYFGPSLKIRARGPPKAAI |
| | | GRELKKAHRKKAYAERKKARRKRAELARSQARGAAAHAAIRERDIPPM |
| | | AYERTQGRNDVTTIPIAAAIKIAATRGARPLPAPKPMKWQCSLYWNEGQ |
| | | RWIRGGMLTAQAYAHAANIHRPMRCEMWGVGNPLKVRAFEGRVADPD |
| | | GAKGRKAEFRLQTNAFYVSGAAYRNKKFKPFGTDRGGIGSARKKRERL |
| | | MAQLAKILDKVVSQAAHSPLDDIWHTRPAQKLRAMIKQLEHEWMFLRP |
| | | QAPTVEGTKPDVDVAGNMQRQIKALMAPDLPPIEKGSPAKRFTGDKRK |
| | | KGERAVRVAEAHSDEVVTAWISRWGIQTRRNEGSYAAQELELLLNWLQ |
| | | ICRRRWLDMTAAQRVSPYIRMKSGRMITDAADEGVAPIPLVENM |
| Cas14 | 191 | KSISGRSIKHMACLKDMLKSEITEIEEKQKKESLRKWDYYSKFSDEILFRR |
| ortholog 85 | | NLNVSANHDANACYGCNPCAFLKEVYGFRIERRNNERIISYRRGLAGCK |
| | | SCVQSTGYPPIEFVRRKFGADKAMEIVREVLHRRNWGALARNIGREKEA |
| | | DPILGELNELLLVDARPYFGNKSAANETNLAFNVITRAAKKFRDEGMYD |
| | | IHKQLDIHSEEGKVPKGRKSRLIRIERKHKAIHGLDPGETWRYPHCGKGE |
| | | KYGVWLNRSRLIHIKGNEYRCLTAFGTTGRRMSLDVACSVLGHPLVKK |
| | | KRKKGKKTVDGTELWQIKKATETLPEDPIDCTFYLYAAKPTKDPFILKV |
| | | GSLKAPRWKKLHKDFFEYSDTEKTQGQEKGKRVVRRGKVPRILSLRPD |
| | | AKFKVSIWDDPYNGKNKEGTLLRMELSGLDGAKKPLILKRYGEPNTKPK |
| | | NFVFWRPHITPHPLTFTPKHDFGDPNKKTKRRRVFNREYYGHLNDLAK |
| | | MEPNAKFFEDREVSNKKNPKAKNIRIQAKESLPNIVAKNGRWAAFDPND |
| | | SLWKLYLHWRGRRKTIKGGISQEFQEFKERLDLYKKHEDESEWKEKEK |
| | | LWENHEKEWKKTLEIHGSIAEVSQRCVMQSMMGPLDGLVQKKDYVHI |
| | | GQSSLKAADDAWTFSANRYKKATGPKWGKISVSNLLYDANQANAELIS |
| | | QSISKYLSKQKDNQGCEGRKMKFLIKIIEPLRENFVKHTRWLHEMTQKD |
| | | CEVRAQFSRVSM |
| Cas14 | 192 | FPSDVGADALKHVRMLQPRLTDEVRKVALTRAPSDRPALARFAAVAQD |
| ortholog 86 | | GLAFVRHLNVSANHDSNCTFPRDPRDPRRGPCEPNPCAFLREVWGFRIV |
| | | ARGNERALSYRRGLAGCKSCVQSTGFPSVPFHRIGADDCMRKLHEILKA |
| | | RNWRLLARNIGREREADPLLTELSEYLLVDARTYPDGAAPNSGRLAENV |
| | | IKRAAKKFRDEGMRDIHAQLRVHSREGKVPKGRLQRLRRIERKHRAIHA |
| | | LDPGPSWEAEGSARAEVQGVAVYRSQLLRVGHHTQQIEPVGIVARTLFG |
| | | VGRTDLDVAVSVLGAPLTKRKKGSKTLESTEDFRIAKARETRAEDKIEV |
| | | AFVLYPTASLLRDEIPKDAFPAMRIDRFLLKVGSVQADREILLQDDYYRF |
| | | GDAEVKAGKNKGRTVTRPVKVPRLQALRPDAKFRVNVWADPFGAGDS |
| | | PGTLLRLEVSGVTRRSQPLRLLRYGQPSTQPANFLCWRPHRVPDPMTFTP |
| | | RQKFGERRKNRRTRRPRVFERLYQVHIKHLAHLEPNRKWFEEARVSAQ |
| | | KWAKARAIRRKGAEDIPVVAPPAKRRWAALQPNAELWDLYAHDREAR |
| | | KRFRGGRAAEGEEFKPRLNLYLAHEPEAEWESKRDRWERYEKKWTAV |
| | | LEEHSRMCAVADRTLPQFLSDPLGARMDDKDYAFVGKSALAVAEAFVE |
| | | EGTVERAQGNCSITAKKKFASNASRKRLSVANLLDVSDKADRALVFQA |
| | | VRQYVQRQAENGGVEGRRMAFLRKLLAPLRQNFVCHTRWLHM |
| Cas14 | 193 | AARKKKRGKIGITVKAKEKSPPAAGPFMARKLVNVAANVDGVEVHLCV |
| ortholog 87 | | ECEADAHGSASARLLGGCRSCTGSIGAEGRLMGSVDVDRERVIAEPVHT |
| | | ETERLGPDVKAFEAGTAESKYAIQRGLEYWGVDLISRNRARTVRKMEE |
| | | ADRPESSTMEKTSWDEIAIKTYSQAYHASENHLFWERQRRVRQHALALF |
| | | RRARERNRGESPLQSTQRPAPLVLAALHAEAAAISGRARAEYVLRGPSA |
| | | NVRAAAADIDAKPLGHYKTPSPKVARGFPVKRDLLRARHRIVGLSRAYF |
| | | KPSDVVRGTSDAIAHVAGRNIGVAGGKPKEIEKTFTLPFVAYWEDVDRV |
| | | VHCSSFKADGPWVRDQRIKIRGVSSAVGTFSLYGLDVAWSKPTSFYIRCS |
| | | DIRKKFHPKGFGPMKHWRQWAKELDRLTEQRASCVVRALQDDEELLQT |
| | | MERGQRYYDVFSCAATHATRGEADPSGGCSRCELVSCGVAHKVTKKA |
| | | KGDTGIEAVAVAGCSLCESKLVGPSKPRVHRQMAALRQSHALNYLRRL |
| | | QREWEALEAVQAPTPYLRFKYARHLEVRSM |
| Cas14 | 194 | AAKKKKQRGKIGISVKPKEGSAPPADGPFMARKLVNVAANVDGVEVNL |
| ortholog 88 | | CIECEADAHGSAPARLLGGCKSCTGSIGAEGRLMGSVDVDRADAIAKPV |
| | | NTETEKLGPDVQAFEAGTAETKYALQRGLEYWGVDLISRNRSRTVRRTE |
| | | EGQPESATMEKTSWDEIAIKSYTRAYHASENHLFWERQRRVRQHALALF |
| | | KRAKERNRGDSTLPREPGHGLVAIAALACEAYAVGGRNLAETVVRGPT |
| | | FGTARAVRDVEIASLGRYKTPSPKVAHGSPVKRDFLRARHRIVGLARAY |
| | | YRPSDVVRGTSDAIAHVAGRNIGVAGGKPRAVEAVFTLPFVAYWEDVD |
| | | RVVHCSSFQVSAPWNRDQRMKIAGVTTAAGTFSLHGGELKWAKPTSFY |
| | | IRCSDTRRKFRPKGFGPMKRWRQWAKDLDRLVEQRASCVVRALQDDA |
| | | ALLETMERGQRYYDVFACAVTHATRGEADRLAGCSRCALTPCQEAHRV |
| | | TTKPRGDAGVEQVQTSDCSLCEGKLVGPSKPRLHRTLTLLRQEHGLNYL |
| | | RRLQREWESLEAVQVPTPYLRFKYARHLEVRSM |
| Cas14 | 195 | TDSQSESVPEVVYALTGGEVPGRVPPDGGSAEGARNAPTGLRKQRGKIK |
| ortholog 89 | | ISAKPSKPGSPASSLARTLVNEAANVDGVQSSGCATCRMRANGSAPRAL |
| | | PIGCVACASSIGRAPQEETVCALPTTQGPDVRLLEGGHALRKYDIQRALE |
| | | YWGVDLIGRNLDRQAGRGMEPAEGATATMKRVSMDELAVLDFGKSYY |
| | | ASEQHLFAARQRRVRQHAKALKIRAKHANRSGSVKRALDRSRKQVTAL |
| | | AREFFKPSDVVRGDSDALAHVVGRNLGVSRHPAREIPQTFTLPLCAYWE |
| | | DVDRVISCSSLLAGEPFARDQEIRIEGVSSALGSLRLYRGAIEWHKPTSLY |
| | | IRCSDTRRKFRPRGGLKKRWRQWAKDLDRLVEQRACCIVRSLQADVEL |
| | | LQTMERAQRFYDVHDCAATHVGPVAVRCSPCAGKQFDWDRYRLLAAL |
| | | RQEHALNYLRRLQREWESLEAQQVKMPYLRFKYARKLEVSGPLIGLEV |
| | | RREPSMGTAIAEM |
| Cas14 | 196 | AGTAGRRHGSLGARRSINIAGVTDRHGRWGCESCVYTRDQAGNRARCA |
| ortholog 90 | | PCDQSTYAPDVQEVTIGQRQAKYTIFLTLQSFSWTNTMRNNKRAAAGRS |
| | | KRTTGKRIGQLAEIKITGVGLAHAHNVIQRSLQHNITKMWRAEKGKSKR |
| | | VARLKKAKQLTKRRAYFRRRMSRQSRGNGFFRTGKGGIHAVAPVKIGL |
| | | DVGMIASGSSEPADEQTVTLDAIWKGRKKKIRLIGAKGELAVAACRFRE |
| | | QQTKGDKCIPLILQDGEVRWNQNNWQCHPKKLVPLCGLEVSRKFVSQA |
| | | DRLAQNKVASPLAARFDKTSVKGTLVESDFAAVLVNVTSIYQQCHAML |
| | | LRSQEPTPSLRVQRTITSM |
| Cas14 | 197 | GVRFSPAQSQVFFRTVIPQSVEARFAINMAAIHDAAGAFGCSVCRFEDRT |
| ortholog 91 | | PRNAKAVHGCSPCTRSTNRPDVFVLPVGAIKAKYDVFMRLLGFNWTHL |
| | | NRRQAKRVTVRDRIGQLDELAISMLTGKAKAVLKKSICHNVDKSFKAM |
| | | RGSLKKLHRKASKTGKSQLRAKLSDLRERTNTTQEGSHVEGDSDVALN |
| | | KIGLDVGLVGKPDYPSEESVEVVVCLYFVGKVLILDAQGRIRDMRAKQY |
| | | DGFKIPIIQRGQLTVLSVKDLGKWSLVRQDYVLAGDLRFEPKISKDRKYA |
| | | ECVKRIALITLQASLGFKERIPYYVTKQVEIKNASHIAFVTEAIQNCAENF |
| | | REMTEYLMKYQEKSPDLKVLLTQLM |
| Cas14 | 198 | RAVVGKVFLEQARRALNLATNFGTNHRTGCNGCYVTPGKLSIPQDGEK |
| ortholog 92 | | NAAGCTSCLMKATASYVSYPKPLGEKVAKYSTLDALKGFPWYSLRLNL |
| | | RPNYRGKPINGVQEVAPVSKFRLAEEVIQAVQRYHFTELEQSFPGGRRRL |
| | | RELRAFYTKEYRRAPEQRQHVVNGDRNIVVVTVLHELGFSVGMFNEVE |
| | | LLPKTPIECAVNVFIRGNRVLLEVRKPQFDKERLLVESLWKKDSRRHTA |
| | | KWTPPNNEGRIFTAEGWKDFQLPLLLGSTSRSLRAIEKEGFVQLAPGRDP |
| | | DYNNTIDEQHSGRPFLPLYLYLQGTISQEYCVFAGTWVIPFQDGISPYSTK |
| | | DTFQPDLKRKAYSLLLDAVKHRLGNKVASGLQYGRFPAIEELKRLVRM |
| | | HGATRKIPRGEKDLLKKGDPDTPEWWLLEQYPEFWRLCDAAAKRVSQN |
| | | VGLLLSLKKQPLWQRRWLESRTRNEPLDNLPLSMALTLHLTNEEAL |
| Cas14 | 199 | AAVYSKFYIENHFKMGIPETLSRIRGPSIIQGFSVNENYINIAGVGDRDFIF |
| ortholog 93 | | GCKKCKYTRGKPSSKKINKCHPCKRSTYPEPVIDVRGSISEFKYKIYNKL |
| | | KQEPNQSIKQNTKGRMNPSDHTSSNDGIIINGIDNRIAYNVIFSSYKHLME |
| | | KQINLLRDTTKRKARQIKKYNNSGKKKHSLRSQTKGNLKNRYHMLGMF |
| | | KKGSLTITNEGDFITAVRKVGLDISLYKNESLNKQEVETELCLNIKWGRT |
| | | KSYTVSGYIPLPINIDWKLYLFEKETGLTLRLFGNKYKIQSKKFLIAQLFK |
| | | PKRPPCADPVVKKAQKWSALNAHVQQMAGLFSDSHLLKRELKNRMHK |
| | | QLDFKSLWVGTEDYIKWFEELSRSYVEGAEKSLEFFRQDYFCFNYTKQT |
| | | TM |
| Cas14 | 200 | PQQQRDLMLMAANYDQDYGNGCGPCTVVASAAYRPDPQAQHGCKRH |
| ortholog 94 | | LRTLGASAVTHVGLGDRTATITALHRLRGPAALAARARAAQAASAPMT |
| | | PDTDAPDDRRRLEAIDADDVVLVGAHRALWSAVRRWADDRRAALRRR |
| | | LHSEREWLLKDQIRWAELYTLIEASGTPPQGRWRNTLGALRGQSRWRR |
| | | VLAPTMRATCAETHAELWDALAELVPEMAKDRRGLLRPPVEADALWR |
| | | APMIVEGWRGGHSVVVDAVAPPLDLPQPCAWTAVRLSGDPRQRWGLH |
| | | LAVPPLGQVQPPDPLKATLAVSMRHRGGVRVRTLQAMAVDADAPMQR |
| | | HLQVPLTLQRGGGLQWGIHSRGVRRREARSMASWEGPPIWTGLQLVNR |
| | | WKGQGSALLAPDRPPDTPPYAPDAAVAPAQPDTKRARRTLKEACTVCR |
| | | CAPGHMRQLQVTLTGDGTWRRFRLRAPQGAKRKAEVLKVATQHDERI |
| | | ANYTAWYLKRPEHAAGCDTCDGDSRLDGACRGCRPLLVGDQCFRRYL |
| | | DKIEADRDDGLAQIKPKAQEAVAAMAAKRDARAQKVAARAAKLSEAT |
| | | GQRTAATRDASHEARAQKELEAVATEGTTVRHDAAAVSAFGSWVARK |
| | | GDEYRHQVGVLANRLEHGLRLQELMAPDSVVADQQRASGHARVGYRY |
| | | VLTAM |
| Cas14 | 201 | AVAHPVGRGNAGSPGARGPEELPRQLVNRASNVTRPATYGCAPCRHVR |
| ortholog 95 | | LSIPKPVLTGCRACEQTTHPAPKRAVRGGADAAKYDLAAFFAGWAADL |
| | | EGRNRRRQVHAPLDPQPDPNHEPAVTLQKIDLAEVSIEEFQRVLARSVK |
| | | HRHDGRASREREKARAYAQVAKKRRNSHAHGARTRRAVRRQTRAVRR |
| | | AHRMGANSGEILVASGAEDPVPEAIDHAAQLRRRIRACARDLEGLRHLS |
| | | RRYLKTLEKPCRRPRAPDLGRARCHALVESLQAAERELEELRRCDSPDT |
| | | AMRRLDAVLAAAASTDATFATGWTVVGMDLGVAPRGSAAPEVSPMEM |
| | | AISVFWRKGSRRVIVSKPIAGMPIRRHELIRLEGLGTLRLDGNHYTGAGV |
| | | TKGRGLSEGTEPDFREKSPSTLGFTLSDYRHESRWRPYGAKQGKTARQF |
| | | FAAMSRELRALVEHQVLAPMGPPLLEAHERRFETLLKGQDNKSIHAGGG |
| | | GRYVWRGPPDSKKRPAADGDWFRFGRGHADHRGWANKRHELAANYL |
| | | QSAFRLWSTLAEAQEPTPYARYKYTRVTM |
| Cas14 | 202 | WDFLTLQVYERHTSPEVCVAGNSTKCASGTRKSDHTHGVGVKLGAQEI |
| ortholog 96 | | NVSANDDRDHEVGCNICVISRVSLDIKGWRYGCESCVQSTPEWRSIVRF |
| | | DRNHKEAKGECLSRFEYWGAQSIARSLKRNKLMGGVNLDELAIVQNEN |
| | | VVKTSLKHLFDKRKDRIQANLKAVKVRMRERRKSGRQRKALRRQCRKL |
| | | KRYLRSYDPSDIKEGNSCSAFTKLGLDIGISPNKPPKIEPKVEVVFSLFYQ |
| | | GACDKIVTVSSPESPLPRSWKIKIDGIRALYVKSTKVKFGGRTFRAGQRN |
| | | NRRKVRPPNVKKGKRKGSRSQFFNKFAVGLDAVSQQLPIASVQGLWGR |
| | | AETKKAQTICLKQLESNKPLKESQRCLFLADNWVVRVCGFLRALSQRQG |
| | | PTPYIRYRYRCNM |
| Cas14 | 203 | ARNVGQRNASRQSKRESAKARSRRVTGGHASVTQGVALINAAANADR |
| ortholog 97 | | DHTTGCEPCTWERVNLPLQEVIHGCDSCTKSSPFWRDIKVVNKGYREAK |
| | | EEIMRIASGISADHLSRALSHNKVMGRLNLDEVCILDFRTVLDTSLKHLT |
| | | DSRSNGIKEHIRAVHRKIRMRRKSGKTARALRKQYFALRRQWKAGHKP |
| | | NSIREGNSLTALRAVGFDVGVSEGTEPMPAPQTEVVLSVFYKGSATRILR |
| | | ISSPHPIAKRSWKVKIAGIKALKLIRREHDFSFGRETYNASQRAEKRKFSP |
| | | HAARKDFFNSFAVQLDRLAQQLCVSSVENLWVTEPQQKLLTLAKDTAP |
| | | YGIREGARFADTRARLAWNWVFRVCGFTRALHQEQEPTPYCRFTWRSK |
| | | M |
| CasM | 2435 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| | | FTKSLKDGLMYGRVSLPTYRKDNPLFVDVRFVALRGTKQKYNGLYHEY |
| | | KSHTEFLDNLYSSDLKVYIKFANDITFQVIFGNPRKSSALRSEFQNIFEEY |
| | | YKVCQSSIQFSGTKIILNMAMDIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRCSCCGYED |
| | | AGNRPKKEKGQAYFKCLKCGEEMNADFNAARNIAMSTEFQSGKKTKK |
| | | QKKEQHENK |
| CasΦ.12 | 2592 | MIKPTVSQFLTPGFKLIRNHSRTAGRKLKNEGEEACKKFVRENEIPKDEC |
| L26R | | PNFQGGPAIANIIAKSREFTEWEIYQSSLAIQEVIFTLPKDKLPEPILKEEW |
| | | RAQWLSEHGLDTVPYKEAAGLNLIIKNAVNTYKGVQVKVDNKNKNNL |
| | | AKINRKNEIAKLNGEQEISFEEIKAFDDKGYLLQKPSPNKSIYCYQSVSPK |
| | | PFITSKYHNVNLPEEYIGYYRKSNEPIVSPYQFDRLRIPIGEPGYVPKWQY |
| | | TFLSKKENKRRKLSKRIKNVSPILGIICIKKDWCVFDMRGLLRTNHWKKY |
| | | HKPTDSINDLFDYFTGDPVIDTKANVVRFRYKMENGIVNYKPVREKKGK |
| | | ELLENICDQNGSCKLATVDVGQNNPVAIGLFELKKVNGELTKTLISRHPT |
| | | PIDFCNKITAYRERYDKLESSIKLDAIKQLTSEQKIEVDNYNNNFTPQNTK |
| | | QIVCSKLNINPNDLPWDKMISGTHFISEKAQVSNKSEIYFTSTDKGKTKD |
| | | VMKSDYKWFQDYKPKLSKEVRDALSDIEWRLRRESLEFNKLSKSREQD |
| | | ARQLANWISSMCDVIGIENLVKKNNFFGGSGKREPGWDNFYKPKKENR |
| | | WWINAIHKALTELSQNKGKRVILLPAMRTSITCPKCKYCDSKNRNGEKF |
| | | NCLKCGIELNADIDVATENLATVAITAQSMPKPTCERSGDAKKPVRARK |
| | | AKAPEFHDKLAPSYTVVLREAV |
| CasM. | 2599 | MVITRKIALTVVGNKEEKDRVYTYIRDGIKNQNLAMNQYMSALYVAN |
| 292007 | | MQDISKDDRKELNHLYTRISTSKKGSAYSTDIQFPKGLPCTSSLGQEVRA |
| | | KFKKACKDGLMYGRVSLPTYRANNPLLIHVDYVRLRSTNPHNDTGLYH |
| | | NYESHTEFLEHLYKNDCEVFIKFANNITFQLFFGQPHKSHELRSVIQKVFE |
| | | EYYSVCGSSIEISKKGKIMLNMCIEIPVEKKELDENIVVGVDLGISTPAMC |
| | | GLNCNDYVREGIGSKDTLLSKRTQLQRQYRELQGRMKMTNGGHGRGK |
| | | KLKKMDDYRNHERHFVQTYNHQVSKKIVDFALKYKAKYINVEDLSGFG |
| | | NRDTNQWVLRNWSYYELQQYITYKAQKYGIEVRKVKPYLTSQTCSHCG |
| | | HYEPGQRLDQAHFECKNCGLKINADFNASRNIAMSTEFV |
| CasM. | 2601 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| D220R | | FTKSLKDGLMYGRVSLPTYRKDNPLFVDVRFVALRGTKQKYNGLYHEY |
| | | KSHTEFLDNLYSSDLKVYIKFANDITFQVIFGNPRKSSALRSEFQNIFEEY |
| | | YKVCQSSIQFSGTKIILNMAMDIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRCSCCGYED |
| | | AGNRPKKEKGQAYFKCLKCGEEMNADFNAARNIAMSTEFQSGKKTKK |
| | | QKKEQHENK |

TABLE 1.1 provides illustrative nuclear localization sequences that are useful in the compositions, systems and methods described herein

TABLE 1.1
Exemplary Nuclear Localization Signal Sequences

| SEQ | | |
|---|---|---|
| ID | | |
| NO: | Description | Sequence |
| 1584 | NLS | KRPAATKKAGQAKKKKEF |
| 1585 | NLS | PKKKRKV |
| 1586 | NLS | PAAKRVKLD |
| 1587 | NLS | PKKKRKVGIHGVPAA |
| 2642 | NLS | KR(K/R)R |
| 2643 | NLS | (P/R)XXKR(\hat{ }D/E)(K/R) |
| 2644 | NLS | KRX(W/F/Y)XXAF |
| 2645 | NLS | (R/P)XXKR(K/R)(\hat{ }D/E) |
| 2646 | NLS | LGKR(K/R)(W/F/Y) |
| 2647 | NLS | KRX₁₀K(K/R)(K/R) |
| 2648 | NLS | K(K/R)RK |
| 2649 | NLS | KRX₁₁K(K/R)(K/R) |
| 2650 | NLS | KRX₁₂K(K/R)(K/R) |
| 2651 | NLS | KRX₁₀K(K/R)X(K/R) |
| 2652 | NLS | KRX₁₁K(K/R)X(K/R) |
| 2653 | NLS | KRX₁₂K(K/R)X(K/R) |
| 2654 | NLS | APKKKRKVGIHGVPAA |
| 2655 | NLS | LPPLERLTL |

*wherein X is any naturally occurring amino acid; and \hat{ }D/E is any naturally occurring amino acid except Asp or Glu

TABLE 2 provides illustrative nucleotide sequences (DNA sequences) of repeat sequences that are useful in the compositions, systems and methods described herein.

TABLE 2
Exemplary Repeat Sequences (DNA sequences) for CasΦ Effector Proteins

| | | SEQ ID. |
|---|---|---|
| Name | Repeat sequence (shown as DNA), 5′-to-3′ | NO. |
| CasΦ.01 | GGAGAGATCTCAAACGATTGCTCGATTAGTCGAGAC | 204 |
| CasΦ.02 | GTCGGAACGCTCAACGATTGCCCCTCACGAGGGGAC | 205 |
| CasΦ.04 | ACCAAAACGACTATTGATTGCCCAGTACGCTGGGAC | 206 |
| CasΦ.07 | GGATCCAATCCTTTTTGATTGCCCAATTCGTTGGGAC | 207 |
| CasΦ.10 | GGATCTGAGGATCATTATTGCTCGTTACGACGAGAC | 208 |
| CasΦ.11 | CCTGCGAAACCTTTTGATTGCTCAGTACGCTGAGAC | 209 |
| CasΦ.12 | CTTTCAAGACTAATAGATTGCTCCTTACGAGGAGAC | 210 |
| CasΦ.13 | GTAGAAGACCTCGCTGATTGCTCGGTGCGCCGAGAC | 211 |
| CasΦ.17 | ATGGCAACAGACTCTCATTGCGCGGTACGCCGCGAC | 212 |
| CasΦ.18 | ACCAAAACGACTATTGATTGCCCAGTACGCTGGGAC | 206 |
| CasΦ.19 | GTCGCTCTCTAACGCTTGCCCAGTACGCTGGGAC | 213 |
| CasΦ.20 | GCTGGAAGACTCAATGATGGCTCCTTACGAGGAGAC | 214 |
| CasΦ.21 | GGTTGAACCCTCAACAGATTGCTCGGTAAGCCGAGAC | 215 |
| CasΦ.22 | GGTTGAACCCTCAACAGATTGCTCGGTAAGCCGAGAC | 215 |
| CasΦ.23 | CTTGAAATCCTGTCAGATTGCTCCCTTCGGGGAGAC | 216 |
| CasΦ.24 | GCTGGAAGACTCAATGATGGCTCCTTACGAGGAGAC | 214 |
| CasΦ.25 | GCTGGAAGACTCAATGATGGCTCCTTACGAGGAGAC | 214 |
| CasΦ.26 | CTAGGAACGCACGCAGATTGCTCGGTACGCCGAGAC | 217 |
| CasΦ.27 | ATTGCAACGCCTAAAGATTGCTCGATACGTCGAGAC | 218 |
| CasΦ.28 | GTTCGGCRAYCCTTTGATTGCTCAGTACGCTGAGAC | 219 |
| CasΦ.29 | GTTGAACCTAGATCAGATGGCTCAGTACGCTGAGAC | 220 |
| CasΦ.30 | CCCTCAACACGTCAGAAATGCCCGGCACGCCGGGAC | 221 |
| CasΦ.31 | GTCGCAAGACTCGAATAATTGCCCCTCTATGGGGAC | 222 |
| CasΦ.32 | GCTGGGGACCGATCCTGATTGCTCGCTGCGGCGAGAC | 223 |
| CasΦ.33 | CTCTCAATGGATAACGATTGCTCTCTACGGAGAGAC | 224 |
| CasΦ.34 | GCTGGAAGACTCAATGATGGCTCCTTACGAGGAGAC | 214 |
| CasΦ.35 | GTTGAACCCTCAACAGATTGCTCGGTAAGCCGAGAC | 225 |
| CasΦ.36 | GTCGCAAGACTCGAATAATTGCCCCTCTATGGGGAC | 222 |
| CasΦ.37 | GTCGGAACGCTCAACGATTGCCCCTCACGAGGGGAC | 205 |
| CasΦ.38 | GTTGAACCTAGATCAGATGGCTCAGTACGCTGAGAC | 220 |
| CasΦ.39 | CTCTCAATGGATAACGATTGCTCTCTACGGAGAGAC | 224 |
| CasΦ.41 | ACTGAAACCACCAACGATTGCGCTCCTCGGAGCGAC | 226 |
| CasΦ.42 | ACCAAAACGACTATTGATTGCCCAGTACGCTGGGAC | 206 |
| CasΦ.43 | GTTGAACCTAGATCAGATGGCTCAGTACGCTGAGAC | 220 |
| CasΦ.44 | GTTGAACCCTCAACAGATTGCTCGGTAAGCCGAGAC | 225 |
| CasΦ.45 | GTTGAACCTAGATCAGATGGCTCAGTACGCTGAGAC | 220 |
| CasΦ.46 | GTCGGAACGCTCAACGATTGCCCCTCACGAGGGGAC | 205 |
| CasΦ.47 | GGTTGAACCCTCAACAGATTGCTCGGTAAGCCGAGAC | 215 |
| CasΦ.48 | GGTTGAACCCTCAACAGATTGCTCGGTAAGCCGAGAC | 215 |

TABLE 3 provides illustrative nucleotide sequences (RNA sequences) of repeat sequences that are useful in the compositions, systems and methods described herein.

TABLE 3
Exemplary crRNA Repeat Sequence for CasM Effector Proteins

| | | SEQ |
|---|---|---|
| | | ID |
| Name | Repeat sequence | NO. |
| CasM.298706 | CGUUGCAGCUCGCACGUUGGCACUGGUUGAAGG | 1588 |
| CasM.280604 | GUUGCAACUCACGCGCGUAUGUGGCUUGAAGG | 1589 |
| CasM.281060 | GUUGCAAUUCAUAUCUCCGGGUGGAUUGAAGG | 1590 |
| CasM.284933 | GUUGCAGCGUGCGCGAGCGUGUGGCUUGAAGG | 1591 |
| CasM.287908 | GUUGCAACUCGCACGUGAAUGCGACUUGAAGG | 1592 |
| CasM.288518 | GAUGCAACUCGUGUGUAUGUGCGAGUUGAAGG | 1593 |
| CasM.293891 | GACGCAACUCGCGCGCGGGCAUGUAUUGAGGG | 1594 |
| CasM.294270 | GAUGCAUCUGACACAGCUGGGUGAGUUGAAGG | 1595 |
| CasM.294491 | GUUGCAACACAUGUAUGUGGGUGAGUUGAAGG | 1596 |
| CasM.295047 | GUUGCAGCGUGCGCGAGCGUGUGGCUUGAAGG | 1591 |
| CasM.299588 | GUUGCAAUUUGUAUACGAGUGUGACUUGAAGG | 1597 |
| CasM.277328 | GCUGCAACACGCGCGGGUACGCGGGUUGAAGG | 1598 |
| CasM.297894 | GUUGCAACUCGCACGUUGGCACUGAUUGAAGG | 1599 |
| CasM.291449 | GCUGUAGCCCUGCUCAAAUUGUAGGGCGCAUGCAGG | 1600 |
| CasM.291449 | GCUGUAGCCCUGCUCAAAUUGUAGGGCGCAUGCAGG | 1600 |
| CasM.297599 | GUUGUAGUCGACCUGAAUCUGUGGGGUGCUUACAGG | 1601 |
| CasM.297599 | GUUGUAGUCGACCUGAAUCUGUGGGGUGCUUACAGG | 1601 |
| CasM.286588 | GGUGUAUGUAACCGCAAUUUGAAGGGUGCAUACAGG | 1602 |
| CasM.286588 | GGUGUAUGUAACCGCAAUUUGAAGGGUGCAUACAGG | 1602 |
| CasM.286910 | GUUGGAAUCGACCUUAAUUUGAGGUGUGCUUACAGG | 1603 |
| CasM.286910 | GUUGGAAUCGACCUUAAUUUGAGGUGUGCUUACAGG | 1603 |
| CasM.292335 | GCUGAAAGAGCAGAGAAUUUGUUGUGUGCAUACAGG | 1604 |
| CasM.292335 | GCUGAAAGAGCAGAGAAUUUGUUGUGUGCAUACAGG | 1604 |
| CasM.293576 | GUUGGAGUCGGCUUGAAUCUGCGGGGUGCUUACAGG | 1605 |
| CasM.293576 | GUUGGAGUCGGCUUGAAUCUGCGGGGUGCUUACAGG | 1605 |
| CasM.294537 | GUUGGAAUCGACCUUAAUUUGAGGUGUGCUUACAGG | 1603 |
| CasM.294537 | GUUGGAAUCGACCUUAAUUUGAGGUGUGCUUACAGG | 1603 |
| CasM.298538 | GUUGUAAGAGACCCGAAUUUUAGCUGUGUAUACAGG | 1606 |
| CasM.298538 | GUUGUAAGAGACCCGAAUUUUAGCUGUGUAUACAGG | 1606 |
| CasM.19924 | GUUGUGAAUGCAGGCAUUUUUGAUGGUAAAUCCAAC | 1607 |
| CasM.19952 | ACUGUCAGACAAUGCAAAAUGUGUGGUACAUCCAAC | 1608 |
| CasM.274559 | GCUGUCAGUAGUAGUAAAAAUGGGGGUACAUCCAAC | 1609 |
| CasM.286251 | ACUGUCAGUACAUGCAAAAAUGAGGGUACAUCCAAC | 1610 |
| CasM.288480 | ACUGUCAGACAAUGCAAAAUGAGUGGUACAUCCAAC | 1611 |
| CasM.288668 | GCUGUUAGAACAUACAAAAUGAAAGGUACAUCCAAC | 1612 |
| CasM.289206 | GCUGCAUGUCAUGGCAAAAGGAAAGGUACAUCCAAC | 1613 |
| CasM.290598 | GCUGUCAGACACCUAAAAAAUGAGGGUACAUCCAAC | 1614 |
| CasM.290816 | GCUGUGAGUCACAGUAAAAAUGAAGGUAUAUCCAAC | 1615 |
| CasM.295071 | ACUGUCAGUACAUGCAAAAAUGAGGGUACAUCCAAC | 1610 |
| CasM.295231 | GCUGUGAGUCACAGUAAAAAUGAAGGUAUAUCCAAC | 1615 |
| CasM.292139 | GAUGUAUAUGCUAUGAUUUUGUAUGGUACAUCCAAC | 1616 |
| CasM.292139 | GAUGUAUAUGCUAUGAUUUUGUAUGGUACAUCCAAC | 1616 |
| CasM.279423 | GCUGUCAGUAGUAGUAAAAAUGGGGGUACAUCCAAC | 1609 |
| CasM.20054 | GUUGAGCUCUGCAUUACGCAGAUGAAUGACGAG | 1617 |
| CasM.20054 | GUUGAGCUCUGCAUUACGCAGAUGAAUGACGAG | 1617 |
| CasM.282673 | GAUGCAACUUAGAUGCAUAUGUAAGUUGUGAG | 1618 |
| CasM.282673 | GAUGCAACUUAGAUGCAUAUGUAAGUUGUGAG | 1618 |
| CasM.282952 | GUUGCAAUCUGCGUACAGGCGUAAGAUGUGAG | 1619 |
| CasM.282952 | GUUGCAAUCUGCGUACAGGCGUAAGAUGUGAG | 1619 |
| CasM.283262 | GAUCAUAUCUGCUUGUAUGGGUAUGCUGCGAG | 1620 |
| CasM.283262 | GAUCAUAUCUGCUUGUAUGGGUAUGCUGCGAG | 1620 |
| CasM.284833 | GUUGCAACUUACGCAUAGGUGUAAAAUACGAG | 1621 |
| CasM.284833 | GUUGCAACUUACGCAUAGGUGUAAAAUACGAG | 1621 |
| CasM.287700 | GAUUAUAUCUGCUUGUAUGGGUAUACUGCGAG | 1622 |
| CasM.291507 | GUUGCAACUUACGCAUAGGUGUAAAAUACGAG | 1621 |
| CasM.291507 | GUUGCAACUUACGCAUAGGUGUAAAAUACGAG | 1621 |
| CasM.293410 | UCAGCUCACAACCUACAUAUGCAUACAAGAUAUAUCGU | 1623 |
| CasM.293410 | UCAGCUCACAACCUACAUAUGCAUACAAGAUAUAUCGU | 1623 |
| CasM.295105 | GAUCAUAUCUGCUUGUAUGGGUAUGCUGCGAG | 1620 |
| CasM.295105 | GAUCAUAUCUGCUUGUAUGGGUAUGCUGCGAG | 1620 |
| CasM.295187 | GAUAUAUCUUGUAUGCAUAUGUAGGUUGUGAG | 1624 |
| CasM.295187 | GAUAUAUCUUGUAUGCAUAUGUAGGUUGUGAG | 1624 |
| CasM.295929 | GUUGCAAUGAACGUAUGUGCAUGAGGUGUGAG | 1625 |
| CasM.295929 | GUUGCAAUGAACGUAUGUGCAUGAGGUGUGAG | 1625 |
| CasΦ.01 | GGAGAGAUCUCAAACGAUUGCUCGAUUAGUCGAGAC | 2073 |
| CasΦ.02 | GUCGGAACGCUCAACGAUUGCCCCUCACGAGGGGAC | 2074 |
| CasΦ.04 | ACCAAAACGACUAUUGAUUGCCCAGUACGCUGGGAC | 2075 |
| CasΦ.07 | GGAUCCAAUCCUUUUUGAUUGCCCAAUUCGUUGGGAC | 2076 |
| CasΦ.10 | GGAUCUGAGGAUCAUUAUUGCUCGUUACGACGAGAC | 2077 |
| CasΦ.11 | CCUGCGAAACCUUUUGAUUGCUCAGUACGCUGAGAC | 2078 |
| CasΦ.12 | CUUUCAAGACUAAUAGAUUGCUCCUUACGAGGAGAC | 2079 |
| CasΦ.13 | GUAGAAGACCUCGCUGAUUGCUCGGUGCGCCGAGAC | 2080 |
| CasΦ.17 | AUGGCAACAGACUCUCAUUGCGCGGUACGCCGCGAC | 2081 |
| CasΦ.18 | ACCAAAACGACUAUUGAUUGCCCAGUACGCUGGGAC | 2075 |
| CasΦ.19 | GUCGCUCUCUAACGCUUGCCCAGUACGCUGGGAC | 2082 |
| CasΦ.20 | GCUGGAAGACUCAAUGAUGGCUCCUUACGAGGAGAC | 2083 |
| CasΦ.21 | GGUUGAACCCUCAACAGAUUGCUCGGUAAGCCGAGAC | 2084 |
| CasΦ.22 | GGUUGAACCCUCAACAGAUUGCUCGGUAAGCCGAGAC | 2084 |
| CasΦ.23 | CUUGAAAUCCUGUCAGAUUGCUCCCUUCGGGGAGAC | 2085 |
| CasΦ.24 | GCUGGAAGACUCAAUGAUGGCUCCUUACGAGGAGAC | 2083 |
| CasΦ.25 | GCUGGAAGACUCAAUGAUGGCUCCUUACGAGGAGAC | 2083 |
| CasΦ.26 | CUAGGAACGCACGCAGAUUGCUCGGUACGCCGAGAC | 2086 |
| CasΦ.27 | AUUGCAACGCCUAAAGAUUGCUCGAUACGUCGAGAC | 2087 |
| CasΦ.28 | GUUCGGCRAYCCUUUGAUUGCUCAGUACGCUGAGAC | 2088 |
| CasΦ.29 | GUUGAACCUAGAUCAGAUGGCUCAGUACGCUGAGAC | 2089 |
| CasΦ.30 | CCCUCAACACGUCAGAAAUGCCCGGCACGCCGGGAC | 2090 |
| CasΦ.31 | GUCGCAAGACUCGAAUAAUUGCCCCUCUAUGGGGAC | 2091 |
| CasΦ.32 | GCUGGGGACCGAUCCUGAUUGCUCGCUGCGGCGAGAC | 2092 |
| CasΦ.33 | CUCUCAAUGGAUAACGAUUGCUCUCUACGGAGAGAC | 2093 |
| CasΦ.34 | GCUGGAAGACUCAAUGAUGGCUCCUUACGAGGAGAC | 2083 |
| CasΦ.35 | GUUGAACCCUCAACAGAUUGCUCGGUAAGCCGAGAC | 2094 |
| CasΦ.36 | GUCGCAAGACUCGAAUAAUUGCCCCUCUAUGGGGAC | 2091 |
| CasΦ.37 | GUCGGAACGCUCAACGAUUGCCCCUCACGAGGGGAC | 2074 |
| CasΦ.38 | GUUGAACCUAGAUCAGAUGGCUCAGUACGCUGAGAC | 2089 |
| CasΦ.39 | CUCUCAAUGGAUAACGAUUGCUCUCUACGGAGAGAC | 2093 |
| CasΦ.41 | ACUGAAACCACCAACGAUUGCGCUCCUCGGAGCGAC | 2095 |
| CasΦ.42 | ACCAAAACGACUAUUGAUUGCCCAGUACGCUGGGAC | 2075 |
| CasΦ.43 | GUUGAACCUAGAUCAGAUGGCUCAGUACGCUGAGAC | 2089 |
| CasΦ.44 | GUUGAACCCUCAACAGAUUGCUCGGUAAGCCGAGAC | 2094 |
| CasΦ.45 | GUUGAACCUAGAUCAGAUGGCUCAGUACGCUGAGAC | 2089 |
| CasΦ.46 | GUCGGAACGCUCAACGAUUGCCCCUCACGAGGGGAC | 2074 |
| CasΦ.47 | GGUUGAACCCUCAACAGAUUGCUCGGUAAGCCGAGAC | 2084 |
| CasΦ.48 | GGUUGAACCCUCAACAGAUUGCUCGGUAAGCCGAGAC | 2084 |
| CasΦ.12 | AUUGCUCCUUACGAGGAGAC | 2656 |

TABLE 4 provides illustrative intermediary sequences that are useful in the compositions, systems and methods described herein.

TABLE 4
Exemplary intermediary sequence for CasM Effector Proteins

| Name | tracrRNA sequence |
|---|---|
| CasM.298706 | GGGGCGUCUUCCCGUCCCUAAAUCGAGAUAGCAGCCAUUUUUCUUCAU |
| | UUUUGAAGACGGUCUUGCACUCGAAAAGGUCAAG (SEQ ID NO: 385) |
| CasM.280604 | GGGGCGACUUCCCGCCCCAAAAUCGAGAAAGUGACUGUCAGACUUUGC |
| | UAUGCAAAGCAAGUAAUACACUCGAGAAGGUAAAGA (SEQ ID NO: 386) |
| CasM.281060 | AGGGCGACUUCCCGUCCUAAAAUCGAGAAAGUGACAAUUCAGUCUCGC |
| | AUUUCGAGCAUUGUAAUACACUCGAAAAGGUUAAG (SEQ ID NO: 387) |
| CasM.284933 | GGGGCGACUUCCCGUCCCAAAAUCGAGAAAGUGGUCGUAAGUCUCGAU |
| | CGGAUCGAAGCAGACAAUACACUCGAAAAGGUUAAGU (SEQ ID NO: 388) |
| CasM.287908 | GGGGCGACUUCCCGUCCCUAAAUCGAGAAAGUGGCGGUAAGACUUCGG |
| | UCUUCGAAGCGCGCAAUACACUCGAAAAGGUUAA (SEQ ID NO: 389) |
| CasM.288518 | GGGGCGACUUCCCGUCCCAAAAUCGAGAAAGUGACAGUAAUUCUUUGU |
| | UUUACAGAGGUUGUAAUACACUCGAUAAGGUUAAG (SEQ ID NO: 390) |
| CasM.293891 | GGGGCGACCUCCCGUCCCAAAAUCGAGAAAGUGGCCGUCAGACUUCUC |
| | GCUGAGAAGCACGCAAUACACUCGAAAAGGUAAAG (SEQ ID NO: 391) |
| CasM.294270 | AGGGCGACUUCCCGUCCUGAAAUCGAGAAAGUGACAAGGAAAGCGCAA |
| | UUUUGCGCCGUUGUAAUACACUCGAGAAGGUCAAG (SEQ ID NO: 392) |
| CasM.294491 | AGGGCGACUUCCCGUCCUAAAAUCGAGAUAGUGACAAGUCAGUCUCUU |
| | AUGAGGAGCAUUGUAAUACACUCGAGAAGGUCAAG (SEQ ID NO: 393) |
| CasM.295047 | GGGGCGACUUCCCGUCCCAAAAUCGAGAAAGUGGUCGUAAGUCUCGAU |
| | CGGAUCGAAGCAGACAAUACACUCGAAAAGGUUAAGU (SEQ ID NO: 388) |
| CasM.299588 | AGGGCGACUUCACGUCCUCAAAUCGAGAAAGUGAGCGUAAGACUUGGC |
| | UUCUGUCAAGCGGUUAAUACACUCGAGAAGGUUAA (SEQ ID NO: 394) |
| CasM.277328 | GGGGCGACUUCCCGUCCCGAAAUCGAGAAAGUGACCGUCAGACUCUGC |
| | UUUGCAGAGCAGGUAAUACACUCGAGAAGGUAAAG (SEQ ID NO: 395) |
| CasM.297894 | GGGGCGUCUUCCCGUCCCUAAAUCGAGAUAGCAGCCAUUUUUCUUCAU |
| | UUUUUGAAGACGGUCUUGCACUCGAAAAGGUCAAG (SEQ ID NO: 396) |
| CasM.291449 | CACGCTAGCTGAAAAGCAACCGCGTACACGCGGACGAACGGCCGACCTG |
| | CTCGGCCTGAAGGTTGAGAAGGTTATGTATAAGAGGAGAAAATCCCCCTT |
| | CATAATCGCTCACCAAGCTCCCAATTTACATATTTT (SEQ ID NO: 397) |
| CasM.291449 | CGGCCGACCUGCUCGGCCUGAAGGUUGAGAAGGUUAUGUAUAAGAGGA |
| | GAAAAUCCCCCUUCAUAAUCGCUCACCAAGCUCCCAAUUUACAUAUUU |
| | U (SEQ ID NO: 398) |
| CasM.297599 | TATTGCGCTAGCCATAATGGCAATCGCGTACAGGCAACTGAAGGCCGACC |
| | TGTACGGCCTTAAGGTTGAGAAGGCACATGTAAGTGGAAAAATGCTTTCC |
| | CGTTGTGTTCGCTCACCAAGCACACACGTTTTTTT (SEQ ID NO: 399) |
| CasM.297599 | GAAGGCCGACCUGUACGGCCUUAAGGUUGAGAAGGCACAUGUAAGUGG |
| | AAAAAUGCUUUCCCGUUGUGUUCGCUCACCAAGCACACACGUUUUUUU |
| | (SEQ ID NO: 400) |
| CasM.286588 | AGGTCGCCGTTTACGTTGCGTCACAAGGGCGCGCGGGCGACCGAAGGCC |
| | GATCTGTACGGCCTGCAGGTTGAGAAGGCACATATTAGAGGAAAATTGCT |
| | TCCCTTTGTGTTCGCTCACCGAGTATTCCTTGTTTTTT (SEQ ID NO: 401) |
| CasM.286588 | AUCUGUACGGCCUGCAGGUUGAGAAGGCACAUAUUAGAGGAAAAUUGC |
| | UUCCCUUUGUGUUCGCUCACCGAGUAUUCCUUGUUUUUU (SEQ ID NO: |
| | 402) |
| CasM.286910 | CAATGTTTCGCTAACCTTTAAGGTAATCGCGGGCAGGCGACTGAAGGCCG |
| | ACCTGTACGGCCTTAAGGCTGAGAAGGCACATGTAAGTGGAAAAATGCT |
| | TTCCCGTTGTGTTCGCTCACCAAGCACATTTGTTTTTTT (SEQ ID NO: 403) |
| CasM.286910 | GAAGGCCGACCUGUACGGCCUUAAGGCUGAGAAGGCACAUGUAAGUGG |
| | AAAAAUGCUUUCCCGUUGUGUUCGCUCACCAAGCACAUUUGUUUUUUU |
| | (SEQ ID NO: 404) |
| CasM.292335 | AGGCCGTTATCAACGTTTCGCGGAAGAGCGGACGAACGGCTGAAGGCCG |
| | ACCTGTACGGCCTAAAGGTTGAGAAGGCACATGTAAGAGGAAAATCGCT |
| | TCCCTTTGTGTTCGCTCACCGGGTACACGCGTTTTTTT (SEQ ID NO: 405) |
| CasM.292335 | AGGCCGACCUGUACGGCCUAAAGGUUGAGAAGGCACAUGUAAGAGGAA |
| | AAUCGCUUCCCUUUGUGUUCGCUCACCGGGUACACGCGUUUUUUU (SEQ |
| | ID NO: 406) |
| CasM.293576 | TCGTAAATGTTGCGCTAGCCATAATGGCAATCGCGTACAGGCAACTGAAG |
| | GCCGACCTGTACGGCCTTAAGGTTGAGAAGGCACATGTCAGTGGAAAAA |
| | TGCTTTCCCTTTGTGTTCGCTCACCAAGCACACGCGGTTTTTT (SEQ ID NO: |
| | 407) |
| CasM.293576 | AAGGCCGACCUGUACGGCCUUAAGGUUGAGAAGGCACAUGUCAGUGGA |
| | AAAAUGCUUUCCCUUUGUGUUCGCUCACCAAGCACACGCGGUUUUUU |
| | (SEQ ID NO: 408) |
| CasM.294537 | AATGTTTCGCTAACCTTTAAGGTAATCGCGGGCAGGCGACTGAAGGCCGA |
| | CCTGTACGGCCTTAAGGCTGAGAAGGCACATGTAAGTGGAAAAATGCTTT |
| | CCCGTTGTGTTCGCTCACCAAGCACATTTGTTTTTTT (SEQ ID NO: 409) |
| CasM.294537 | AAGGCCGACCUGUACGGCCUUAAGGCUGAGAAGGCACAUGUAAGUGGA |
| | AAAAUGCUUUCCCGUUGUGUUCGCUCACCAAGCACAUUUGUUUUUUU |
| | (SEQ ID NO: 410) |
| CasM.298538 | GGTCGTTGTAAAACGTAACGCTAGCCTTATGGCAATCGCGAACGAACGAC |
| | TGAAGGCCGACCTGTACGGCCTGAAGGATGAGAAGGCACATATTAGAGG |
| | AAAAAAATGGTTCCCTTTGTGACCGCTCACCAAACACATGTTTATTTTT |
| | (SEQ ID NO: 411) |
| CasM.298538 | AAGGCCGACCUGUACGGCCUGAAGGAUGAGAAGGCACAUAUUAGAGGA |
| | AAAAAAUGGUUCCCUUUGUGACCGCUCACCAAACACAUGUUUAUUUUU |
| | (SEQ ID NO: 412) |
| CasM.19924 | AUGAAUAGGAUUCGUCCUAUGGGGCAGUUGGUUGCCCUUAGCCUGAGG |
| | CAUUUAUUGCACUCGGGAAGUACCAUUUCUCA (SEQ ID NO: 413) |
| CasM.19952 | AUGAAUAGGAUUCGUCCUAUGGGGCAGUUGGUUGCCCUUAGCCUGAGG |
| | CAUUUAUUGCACUCGGGAAGUACCAUUUCUCA (SEQ ID NO: 413) |
| CasM.274559 | AUGAAUAGGAUUUAUCCUAUGGGGCAGUUGGUUGCCCUUAGCCUGAGG |
| | CAUUUAAUGCACUCGGGAAGUACCUUUUCUCA (SEQ ID NO: 414) |
| CasM.286251 | AAGAAUAGGAUUCAUCCUAUGGGGCAGUUGGUUGCCCUUAGCCUGAGG |
| | AAUUUAAUUCACUCGGGAAGUACCUUUCUCAU (SEQ ID NO: 415) |
| CasM.288480 | AUGAAUAGGAUUCGUCCUAUGGGGCAGUUGGUUGCCCUUAGCCUGAGG |
| | CAUUUAUUGCACUCGGGAAGUACCAUUUCUCA (SEQ ID NO: 413) |
| CasM.288668 | AUGGAUAGGAUUCGUCCUAUGGGGCAGUUGGGACCAUGUAAUGCCCUU |
| | AGCCUGAGGAAUUCAUUUCACUCGGGAAGUAU (SEQ ID NO: 416) |
| CasM.289206 | AUGAAUAGGAUUUAUCCUAUGGGGCAGUUGGUUGCCCUUAGCCUGAGG |
| | CAUUUAAUGCACUCGGGAAGUACCUUUUCUCA (SEQ ID NO: 414) |
| CasM.290598 | AUGAAUAGGAUUUAUCCUAUGGGGCAGUUGGUUGCCCUUAGCCUGAGG |
| | CAUUUAAUGCACUCGGGAAGUACCUUUUCUCA (SEQ ID NO: 414) |
| CasM.290816 | AUGAAUAGGAUUUAUCCUAUGGGGCAGUUGGAUGCCCUUAGCCUGAGG |
| | CAUUUAUUGCACUCGGGAAGUACCUUUUCUCA (SEQ ID NO: 417) |
| CasM.295071 | AAGAAUAGGAUUCAUCCUAUGGGGCAGUUGGUUGCCCUUAGCCUGAGG |
| | AAUUUAAUUCACUCGGGAAGUACCUUUCUCAU (SEQ ID NO: 415) |
| CasM.295231 | AUGAAUAGGAUUUAUCCUAUGGGGCAGUUGGAUGCCCUUAGCCUGAGG |
| | CAUUUAUUGCACUCGGGAAGUACCUUUUCUCA (SEQ ID NO: 417) |
| CasM.292139 | UAUUUUCUAAUGGGGUUGUUGGAAAGAGCUUUUACUGAAAUUUGUAA |
| | AGGUGCCCUGAACUUGAGAAUUGAAAAAUUACUCGAG (SEQ ID NO: 418) |
| CasM.292139 | AUGGGGUUGUUGGAAAGAGCUUUUACUGAAAUUUGUAAAGGUGCCCU |
| | GAACUUGAGAAUUGAAAAAUUACUCGAG (SEQ ID NO: 419) |
| CasM.279423 | AUGAAUAGGAUUUAUCCUAUGGGGCAGUUGGUUGCCCUUAGCCUGAGG |
| | CAUUUAAUGCACUCGGGAAGUACCUUUUCUCA (SEQ ID NO: 414) |
| CasM.20054 | TTCGGGCGGCTCGGCGTCCGTAAATCGAGAAAGAGCTTGTAATTCCTGAT |
| | TCTATCAGGTGAAGCAACACTCGGTAAGGTATAACAATACACATGTATAA |
| | TCCGTGTATTTAAGTTCATTTT (SEQ ID NO: 420) |
| CasM.20054 | UUCGGGCGGCUCGGCGUCCGUAAAUCGAGAAAGAGCUUGUAAUUCCUG |
| | AUUCUAUCAGGUGAAGCAACACUCGGUAAGGUAUAAC (SEQ ID NO: 421) |
| CasM.282673 | ATAAGGGCGGCTCAGCGTCCTAAAGTCGAGAAAGTATGCGTAAACTTCTT |
| | TCATAGAATTGCAGATACTCTCGGCAAGGTAAAAACCCTACAAATTTAAT |
| | CCTTGTAGGCGACTTATATTTGTGTATATTT (SEQ ID NO: 422) |
| CasM.282673 | AUAAGGGCGGCUCAGCGUCCUAAAGUCGAGAAAGUAUGCGUAAACUUC |
| | UUUCAUAGAAUUGCAGAUACUCUCGGCAAGGUAAAA (SEQ ID NO: 423) |
| CasM.282952 | ATTCTTTCCTCGGAAAGTGGTAGATACTCTCGGTAAGGTAAACTGTGTAT |
| | GAACAGTTTGAAATCCTGCACATAAAATCCGTGCAGGCATCTTATAGTTT |
| | TGTGCATCTTT (SEQ ID NO: 424) |
| CasM.282952 | AUUCUUUCCUCGGAAAGUGGUAGAUACUCUCGGUAAGGUAAACUGUGU |
| | AUGAACAGUUUGAAAUCCUGCACAUAAAAUCCGUGCAGGCAUC (SEQ ID |
| | NO: 425) |
| CasM.283262 | TTCGGGCGGCTCGGCGTCCGTAAACCGAGAAAGTATATGTAAGTCTGAAT |
| | TTATTCAGCGTTAGATACACTCGGTAAGGTTCAAACAATACATATTCAAT |
| | CCATGTATTCAGTATATTTGTACATTTTT (SEQ ID NO: 426) |
| CasM.283262 | UUCGGGCGGCUCGGCGUCCGUAAACCGAGAAAGUAUAUGUAAGUCUGA |
| | AUUUAUUCAGCGUUAGAUACACUCGGUAAGGUUCAAAC (SEQ ID NO: |
| | 427) |
| CasM.284833 | TTCAGGGCGACTCGGCGTCCTAAAATCGAGAAAGTGTACATAAATTTTTA |
| | ACAAAATACGGTAAATACTCTCGGTAAGGTTTTAACGTGCACATAATAAT |
| | CCGTGCAACAGGGTTACACTTTTGTGCAATTTT (SEQ ID NO: 428) |
| CasM.284833 | UUCAGGGCGACUCGGCGUCCUAAAAUCGAGAAAGUGUACAUAAAUUUU |
| | UAACAAAAUACGGUAAAUACUCUCGGUAAGGUUUUAAC (SEQ ID NO: |
| | 429) |
| CasM.287700 | UUCGGGCGGCUCGGCGUCCGUAAACCGAGAAAGUAUAUGUAAGUCUGA |
| | AUUUAUUCAGCGUUAGAUACACUCGGUAAGGUUUAAAC (SEQ ID NO: |
| | 430) |
| CasM.291507 | TTCAGGGCGACTCGGCGTCCTAAAATCGAGAAAGTGTACATAAGTTTTTA |
| | ACAAAATACGGTAAATACTCTCGGTAAGGTTTTAACGTGCACATAATAAT |
| | CCGTGCAACAGGGTTACACTTTTGTGCAATTTT (SEQ ID NO: 431) |
| CasM.291507 | UUCAGGGCGACUCGGCGUCCUAAAAUCGAGAAAGUGUACAUAAGUUUU |
| | UAACAAAAUACGGUAAAUACUCUCGGUAAGGUUUUAACG (SEQ ID NO: |
| | 432) |
| CasM.293410 | TATTAAGGGCGGCTCAGCGTCCTTAAGTCGAGAAAGTATACATAAATTTC |
| | TTATATAGAATAGTAGATACTCTCGGCAAGGTATAAACCCTACAAATTTA |
| | ATCCTTGTAGGCAACTTATATTTGTATTTATTT (SEQ ID NO: 433) |
| CasM.293410 | UAUUAAGGGCGGCUCAGCGUCCUUAAGUCGAGAAAGUAUACAUAAAUU |
| | UCUUAUAUAGAAUAGUAGAUACUCUCGGCAAGGUAUAAACC (SEQ ID |
| | NO: 434) |
| CasM.295105 | TTTCGGGCGGCTCGGCGTCCGTAAACCGAGAAAGTATATGTAAGTCTGAA |
| | TTTATTCAGCGTTAGATACACTCGGTAAGGTTCAAACAATACATATTCAA |
| | TCCATGTATTCAGTATATTTGTACATTTTT (SEQ ID NO: 435) |
| CasM.295105 | UUUCGGGCGGCUCGGCGUCCGUAAACCGAGAAAGUAUAUGUAAGUCUG |
| | AAUUUAUUCAGCGUUAGAUACACUCGGUAAGGUUCAAAC (SEQ ID NO: |
| | 436) |
| CasM.295187 | ATATTAAGGGCGGCTCAGCGTCCTTAAGTCGAGAAAGTATACATAAATTT |
| | CTTATATAGAATAGTAGATACTCTCGGCAAGGTATAAACCCTACAAATTT |
| | AATCCTTGTAGGCAACTTATATTTGTATTTATTT (SEQ ID NO: 437) |
| CasM.295187 | AUAUUAAGGGCGGCUCAGCGUCCUUAAGUCGAGAAAGUAUACAUAAAU |
| | UUCUUAUAUAGAAUAGUAGAUACUCUCGGCAAGGUAUAAAC (SEQ ID |
| | NO: 438) |
| CasM.295929 | AAACAAGGGCGGCTCAACGTCCTAGAATCGAGAAAGTATGCGTAAGACT |
| | TATTTATTGAGCGGTAGATACTCTCGGTAAGGTATAAATTCCACAATGAA |
| | AATCCTGTGGACACCGTATAATATGTGCATGTTT (SEQ ID NO: 439) |
| CasM.295929 | AAACAAGGGCGGCUCAACGUCCUAGAAUCGAGAAAGUAUGCGUAAGAC |
| | UUAUUUAUUGAGCGGUAGAUACUCUCGGUAAGGUAUAAAUUC (SEQ ID |
| | NO: 440) |

TABLE 5, TABLE 5.1, TABLE 6, TABLE 6.1, TABLE 7, and TABLE 7.1 provide illustrative spacer sequences that are useful in the compositions, systems and methods described herein.

TABLE 5
Spacer sequences of gRNAs (DNA sequences)
targeting human TRAC in T cells

| | Spacer sequence (5′ → 3′), | | SEQ ID |
|---|---|---|---|
| Name | shown as DNA | Target | NO |
| R3040 | TGGATATCTGTGGGACAAGA | TRAC | 227 |
| R3041 | TCCCACAGATATCCAGAACC | TRAC | 228 |
| R3042 | GAGTCTCTCAGCTGGTACAC | TRAC | 229 |
| R3043 | AGAGTCTCTCAGCTGGTACA | TRAC | 230 |
| R3044 | TCACTGGATTTAGAGTCTCT | TRAC | 231 |
| R3045 | AGAATCAAAATCGGTGAATA | TRAC | 232 |
| R3046 | GAGAATCAAAATCGGTGAAT | TRAC | 233 |
| R3047 | ACCGATTTTGATTCTCAAAC | TRAC | 234 |
| R3048 | TTTGAGAATCAAAATCGGTG | TRAC | 235 |
| R3049 | GTTTGAGAATCAAAATCGGT | TRAC | 236 |
| R3050 | TGATTCTCAAACAAATGTGT | TRAC | 237 |
| R3051 | GATTCTCAAACAAATGTGTC | TRAC | 238 |
| R3052 | ATTCTCAAACAAATGTGTCA | TRAC | 239 |
| R3053 | TGACACATTTGTTTGAGAAT | TRAC | 240 |
| R3054 | TCAAACAAATGTGTCACAAA | TRAC | 241 |
| R3055 | GTGACACATTTGTTTGAGAA | TRAC | 242 |
| R3056 | CTTTGTGACACATTTGTTTG | TRAC | 243 |
| R3057 | TGATGTGTATATCACAGACA | TRAC | 244 |
| R3058 | TCTGTGATATACACATCAGA | TRAC | 245 |
| R3059 | GTCTGTGATATACACATCAG | TRAC | 246 |
| R3060 | TGTCTGTGATATACACATCA | TRAC | 247 |
| R3061 | AAGTCCATAGACCTCATGTC | TRAC | 248 |
| R3062 | CTCTTGAAGTCCATAGACCT | TRAC | 249 |
| R3063 | AAGAGCAACAGTGCTGTGGC | TRAC | 250 |
| R3064 | CTCCAGGCCACAGCACTGTT | TRAC | 251 |
| R3065 | TTGCTCCAGGCCACAGCACT | TRAC | 252 |
| R3066 | GTTGCTCCAGGCCACAGCAC | TRAC | 253 |
| R3067 | CACATGCAAAGTCAGATTTG | TRAC | 254 |
| R3068 | GCACATGCAAAGTCAGATTT | TRAC | 255 |
| R3069 | GCATGTGCAAACGCCTTCAA | TRAC | 256 |
| R3070 | AAGGCGTTTGCACATGCAAA | TRAC | 257 |
| R3071 | CATGTGCAAACGCCTTCAAC | TRAC | 258 |
| R3072 | TTGAAGGCGTTTGCACATGC | TRAC | 259 |
| R3073 | AACAACAGCATTATTCCAGA | TRAC | 260 |
| R3074 | TGGAATAATGCTGTTGTTGA | TRAC | 261 |
| R3075 | TTCCAGAAGACACCTTCTTC | TRAC | 262 |
| R3076 | CAGAAGACACCTTCTTCCCC | TRAC | 263 |
| R3077 | CCTGGGCTGGGGAAGAAGGT | TRAC | 264 |
| R3078 | TTCCCCAGCCCAGGTAAGGG | TRAC | 265 |
| R3079 | CCCAGCCCAGGTAAGGGCAG | TRAC | 266 |
| R3080 | TAAAAGGAAAAACAGACATT | TRAC | 267 |
| R3081 | CTAAAAGGAAAAACAGACAT | TRAC | 268 |
| R3082 | TTCCTTTTAGAAAGTTCCTG | TRAC | 269 |
| R3083 | TCCTTTTAGAAAGTTCCTGT | TRAC | 270 |
| R3084 | CCTTTTAGAAAGTTCCTGTG | TRAC | 271 |
| R3085 | CTTTTAGAAAGTTCCTGTGA | TRAC | 272 |
| R3086 | TAGAAAGTTCCTGTGATGTC | TRAC | 273 |
| R3136 | AGAAAGTTCCTGTGATGTCA | TRAC | 274 |
| R3137 | GAAAGTTCCTGTGATGTCAA | TRAC | 275 |
| R3138 | ACATCACAGGAACTTTCTAA | TRAC | 276 |
| R3139 | CTGTGATGTCAAGCTGGTCG | TRAC | 277 |
| R3140 | TCGACCAGCTTGACATCACA | TRAC | 278 |
| R3141 | CTCGACCAGCTTGACATCAC | TRAC | 279 |
| R3142 | TCTCGACCAGCTTGACATCA | TRAC | 280 |
| R3143 | AAAGCTTTTCTCGACCAGCT | TRAC | 281 |
| R3144 | CAAAGCTTTTCTCGACCAGC | TRAC | 282 |
| R3145 | CCTGTTTCAAAGCTTTTCTC | TRAC | 283 |
| R3146 | GAAACAGGTAAGACAGGGGT | TRAC | 284 |
| R3147 | AAACAGGTAAGACAGGGGTC | TRAC | 285 |

TABLE 5.1
Spacer sequences of gRNAs targeting human TRAC in
T cells

| SEQ | | SEQ | |
|---|---|---|---|
| ID | | ID | |
| NO | Spacer Sequence | NO | Spacer Sequence |
| 1962 | UCACAAAGUAAGGAUUCUGA | 2023 | UCACUGGAUUUAGAGUCUCU |
| 1963 | UGGACUUCAAGAGCAACAGU | 2024 | AGAAUCAAAAUCGGUGAAUA |
| 1964 | AUUCUCAAACAAAUGUGUCA | 2025 | GAGAAUCAAAAUCGGUGAAU |
| 1965 | ACUUUGCAUGUGCAAACGCC | 2026 | ACCGAUUUUGAUUCUCAAAC |
| 1966 | CAAACGCCUUCAACAACAGC | 2027 | UUUGAGAAUCAAAAUCGGUG |
| 1967 | UAUAUCACAGACAAAACUGU | 2028 | GUUUGAGAAUCAAAAUCGGU |
| 1968 | AAUCCAGUGACAAGUCUGUC | 2029 | UGAUUCUCAAACAAAUGUGU |
| 1969 | AUGUGUAUAUCACAGACAAA | 2030 | GAUUCUCAAACAAAUGUGUC |
| 1970 | CAUGUGCAAACGCCUUCAAC | 2031 | UGACACAUUUGUUUGAGAAU |
| 1971 | UCACAGACAAAACUGUGCUA | 2032 | UCAAACAAAUGUGUCACAAA |
| 1972 | UAUCACAGACAAAACUGUGC | 2033 | GUGACACAUUUGUUUGAGAA |
| 1973 | UCUGCCUAUUCACCGAUUUU | 2034 | CUUUGUGACACAUUUGUUUG |
| 1974 | GCCUGGAGCAACAAAUCUGA | 2035 | UGAUGUGUAUAUCACAGACA |
| 1975 | CCAGCUGAGAGACUCUAAAU | 2036 | GUCUGUGAUAUACACAUCAG |
| 1976 | CCUAUUCACCGAUUUUGAUU | 2037 | UGUCUGUGAUAUACACAUCA |
| 1977 | CUAGACAUGAGGUCUAUGGA | 2038 | AAGUCCAUAGACCUCAUGUC |
| 1978 | GACUUCAAGAGCAACAGUGC | 2039 | CUCUUGAAGUCCAUAGACCU |
| 1979 | GCACAGUUUUGUCUGUGAUA | 2040 | AAGAGCAACAGUGCUGUGGC |
| 1980 | AGAAUCAAAAUCGGUGAAUA | 2041 | CUCCAGGCCACAGCACUGUU |
| 1981 | CACAUCAGAAUCCUUACUUU | 2042 | GUUGCUCCAGGCCACAGCAC |
| 1982 | UGAUAUACACAUCAGAAUCC | 2043 | GCACAUGCAAAGUCAGAUUU |
| 1983 | ACACAUUUGUUUGAGAAUCA | 2044 | GCAUGUGCAAACGCCUUCAA |
| 1984 | UGACACAUUUGUUUGAGAAU | 2045 | AAGGCGUUUGCACAUGCAAA |
| 1985 | GAGUCUCUCAGCUGGUACAC | 2046 | UUGAAGGCGUUUGCACAUGC |
| 1986 | UUGCUCCAGGCCACAGCACU | 2047 | AACAACAGCAUUAUUCCAGA |
| 1987 | CACAUGCAAAGUCAGAUUUG | 2048 | UGGAAUAAUGCUGUUGUUGA |
| 1988 | UUUGAGAAUCAAAAUCGGUG | 2049 | UUCCAGAAGACACCUUCUUC |
| 1989 | AUAUACACAUCAGAAUCCUU | 2050 | CAGAAGACACCUUCUUCCCC |
| 1990 | GAAUAAUGCUGUUGUUGAAG | 2051 | CCUGGGCUGGGGAAGAAGGU |
| 1991 | UCUGUGAUAUACACAUCAGA | 2052 | UUCCCCAGCCCAGGUAAGGG |
| 1992 | AUGUCAAGCUGGUCGAGAAA | 2053 | CCCAGCCCAGGUAAGGGCAG |
| 1993 | CUCAUGACGCUGCGGCUGUG | 2054 | UAAAAGGAAAAACAGACAUU |
| 1994 | AUCUGCUCAUGACGCUGCGG | 2055 | CUAAAAGGAAAAACAGACAU |
| 1995 | CUCCCUCGCUCCUUCCUCUG | 2056 | UUCCUUUUAGAAAGUUCCUG |
| 1996 | GGCGUGUUGUAUGUCCUGCU | 2057 | UCCUUUUAGAAAGUUCCUGU |
| 1997 | CACAUUCCCUCCUGCUCCCC | 2058 | CCUUUUAGAAAGUUCCUGUG |
| 1998 | CAAGAUUGUAAGACAGCCUG | 2059 | CUUUUAGAAAGUUCCUGUGA |
| 1999 | CAUUGCCCCUCUUCUCCCUC | 2060 | UAGAAAGUUCCUGUGAUGUC |
| 2000 | UAUCUGGGCGUGUUGUAUGU | 2061 | AGAAAGUUCCUGUGAUGUCA |
| 2001 | UGUCCUGCUGCCGAUGCCUU | 2062 | GAAAGUUCCUGUGAUGUCAA |
| 2002 | AGACAGCCUGUGCUCCCUCG | 2063 | ACAUCACAGGAACUUUCUAA |
| 2003 | UUCCCUUAUUGCUGCUUGUC | 2064 | CUGUGAUGUCAAGCUGGUCG |
| 2004 | AUUAAGAUUGCUGAAGAGCU | 2065 | UCGACCAGCUUGACAUCACA |
| 2005 | CCCCCCCGGCAAUGCCACCA | 2066 | CUCGACCAGCUUGACAUCAC |
| 2006 | UCUGGGCGUGUUGUAUGUCC | 2067 | UCUCGACCAGCUUGACAUCA |
| 2007 | UGAUUAAGAUUGCUGAAGAG | 2068 | AAAGCUUUUCUCGACCAGCU |
| 2008 | GGUCCUGCAGAAUGUUGUGA | 2069 | CAAAGCUUUUCUCGACCAGC |
| 2009 | UGCCCCCCCGGCAAUGCCAC | 2070 | CCUGUUUCAAAGCUUUUCUC |
| 2010 | CUGUGUAUCUGGGCGUGUUG | 2071 | GAAACAGGUAAGACAGGGGU |
| 2011 | UUUGGAGAGGGAGAAGAGGG | 2072 | AAACAGGUAAGACAGGGGUC |
| 2012 | CAGGACCUAGAGCCCAAGAG | 1358 | UCCCACAGAUAUCCAGAACC |
| 2013 | CCGUGAAUGUCAGGCAGUGA | 1353 | GAGUCUCUCAGCUGGUACAC |
| 2014 | GAGAGGGAGAAGAGGGGCAA | 1359 | AGAGUCUCUCAGCUGGUACA |
| 2015 | GGGAGCAGGAGGGAAUGUGC | 1360 | AAGUCCAUAGACCUCAUGUC |
| 2016 | CACAGCCAGGGGAGGCUGCA | 1361 | AAGAGCAACAGUGCUGUGGC |
| 2017 | GGAUGGCGGAGGCAGUCUCU | 1362 | GUUGCUCCAGGCCACAGCAC |
| 2018 | UGGGAUGGCGGAGGCAGUCU | 1363 | GCACAUGCAAAGUCAGAUUU |
| 2019 | GCAGCUCUUCAGCAAUCUUA | 1364 | GCAUGUGCAAACGCCUUCAA |
| 2020 | UGGAUAUCUGUGGGACAAGA | 1365 | CUAAAAGGAAAAACAGACAU |
| 2021 | UCCCACAGAUAUCCAGAACC | 1366 | CUCGACCAGCUUGACAUCAC |
| 2022 | AGAGUCUCUCAGCUGGUACA | 2659 | GAGUCUCUCAGCUGGUAC |

TABLE 6
Spacer sequences of gRNAs (DNA sequences)
targeting human B2M in T cells

| | Spacer Sequence | | |
|---|---|---|---|
| | (5′ --> 3′), | | SEQ |
| Name | shown as DNA | Target | ID NO |
| R3087 | AATATAAGTGGAGGCGTCGC | B2M | 286 |
| R3088 | ATATAAGTGGAGGCGTCGCG | B2M | 287 |
| R3089 | AGGAATGCCCGCCAGCGCGA | B2M | 288 |
| R3090 | CTGAAGCTGACAGCATTCGG | B2M | 289 |
| R3091 | GGGCCGAGATGTCTCGCTCC | B2M | 290 |
| R3092 | GCTGTGCTCGCGCTACTCTC | B2M | 291 |
| R3093 | CTGGCCTGGAGGCTATCCAG | B2M | 292 |
| R3094 | TGGCCTGGAGGCTATCCAGC | B2M | 293 |
| R3095 | ATGTGTCTTTTCCCGATATT | B2M | 294 |
| R3096 | TCCCGATATTCCTCAGGTAC | B2M | 295 |
| R3097 | CCCGATATTCCTCAGGTACT | B2M | 296 |
| R3098 | CCGATATTCCTCAGGTACTC | B2M | 297 |
| R3099 | GAGTACCTGAGGAATATCGG | B2M | 298 |
| R3100 | GGAGTACCTGAGGAATATCG | B2M | 299 |
| R3101 | CTCAGGTACTCCAAAGATTC | B2M | 300 |
| R3102 | AGGTTTACTCACGTCATCCA | B2M | 301 |
| R3103 | ACTCACGTCATCCAGCAGAG | B2M | 302 |
| R3104 | CTCACGTCATCCAGCAGAGA | B2M | 303 |
| R3105 | TCTGCTGGATGACGTGAGTA | B2M | 304 |
| R3106 | CATTCTCTGCTGGATGACGT | B2M | 305 |
| R3107 | CCATTCTCTGCTGGATGACG | B2M | 306 |
| R3108 | ACTTTCCATTCTCTGCTGGA | B2M | 307 |
| R3109 | GACTTTCCATTCTCTGCTGG | B2M | 308 |
| R3110 | AGGAAATTTGACTTTCCATT | B2M | 309 |
| R3111 | CCTGAATTGCTATGTGTCTG | B2M | 310 |
| R3112 | CTGAATTGCTATGTGTCTGG | B2M | 311 |
| R3113 | CTATGTGTCTGGGTTTCATC | B2M | 312 |
| R3114 | AATGTCGGATGGATGAAACC | B2M | 313 |
| R3115 | CATCCATCCGACATTGAAGT | B2M | 314 |
| R3116 | ATCCATCCGACATTGAAGTT | B2M | 315 |
| R3117 | AGTAAGTCAACTTCAATGTC | B2M | 316 |
| R3118 | TTCAGTAAGTCAACTTCAAT | B2M | 317 |
| R3119 | AAGTTGACTTACTGAAGAAT | B2M | 318 |
| R3120 | ACTTACTGAAGAATGGAGAG | B2M | 319 |
| R3121 | TCTCTCCATTCTTCAGTAAG | B2M | 320 |
| R3122 | CTGAAGAATGGAGAGAGAAT | B2M | 321 |
| R3123 | AATTCTCTCTCCATTCTTCA | B2M | 322 |
| R3124 | CAATTCTCTCTCCATTCTTC | B2M | 323 |
| R3125 | TCAATTCTCTCTCCATTCTT | B2M | 324 |
| R3126 | TTCAATTCTCTCTCCATTCT | B2M | 325 |
| R3127 | AAAAAGTGGAGCATTCAGAC | B2M | 326 |
| R3128 | CTGAAAGACAAGTCTGAATG | B2M | 327 |
| R3129 | AGACTTGTCTTTCAGCAAGG | B2M | 328 |
| R3130 | TCTTTCAGCAAGGACTGGTC | B2M | 329 |
| R3131 | CAGCAAGGACTGGTCTTTCT | B2M | 330 |
| R3132 | AGCAAGGACTGGTCTTTCTA | B2M | 331 |
| R3133 | CTATCTCTTGTACTACACTG | B2M | 332 |
| R3134 | TATCTCTTGTACTACACTGA | B2M | 333 |
| R3135 | AGTGTAGTACAAGAGATAGA | B2M | 334 |
| R3148 | TACTACACTGAATTCACCCC | B2M | 335 |
| R3149 | AGTGGGGGTGAATTCAGTGT | B2M | 336 |
| R3150 | CAGTGGGGGTGAATTCAGTG | B2M | 337 |
| R3151 | TCAGTGGGGGTGAATTCAGT | B2M | 338 |
| R3152 | TTCAGTGGGGGTGAATTCAG | B2M | 339 |
| R3153 | ACCCCCACTGAAAAAGATGA | B2M | 340 |
| R3154 | ACACGGCAGGCATACTCATC | B2M | 341 |
| R3155 | GGCTGTGACAAAGTCACATG | B2M | 342 |
| R3156 | GTCACAGCCCAAGATAGTTA | B2M | 343 |
| R3157 | TCACAGCCCAAGATAGTTAA | B2M | 344 |
| R3158 | ACTATCTTGGGCTGTGACAA | B2M | 345 |
| R3159 | CCCCACTTAACTATCTTGGG | B2M | 346 |

TABLE 6.1
Spacer sequences of gRNAs targeting human B2M

| SEQ | | SEQ | |
|---|---|---|---|
| ID | | ID | |
| NO | Spacer Sequence | NO | Spacer Sequence |
| 1626 | CUCGCGCUACUCUCUCUUUC | 1695 | AAUAUAAGUGGAGGCGUCGC |
| 1627 | GGUUUCAUCCAUCCGACAUU | 1696 | AUAUAAGUGGAGGCGUCGCG |
| 1628 | CUACACUGAAUUCACCCCCA | 1697 | AGGAAUGCCCGCCAGCGCGA |
| 1629 | UCUCUUGUACUACACUGAAU | 1698 | CUGAAGCUGACAGCAUUCGG |
| 1630 | CUCACGUCAUCCAGCAGAGA | 1699 | GGGCCGAGAUGUCUCGCUCC |
| 1631 | UGUCUGGGUUUCAUCCAUCC | 1700 | GCUGUGCUCGCGCUACUCUC |
| 1632 | CCUGCCGUGUGAACCAUGUG | 1701 | CUGGCCUGGAGGCUAUCCAG |
| 1375 | UCACAGCCCAAGAUAGUUAA | 1702 | UGGCCUGGAGGCUAUCCAGC |
| 1633 | ACUUUGUCACAGCCCAAGAU | 1703 | AUGUGUCUUUUCCCGAUAUU |
| 1634 | UCUGGGUUUCAUCCAUCCGA | 1704 | UCCCGAUAUUCCUCAGGUAC |
| 1635 | AACCAUGUGACUUUGUCACA | 1705 | CCCGAUAUUCCUCAGGUACU |
| 1636 | AAUGCUCCACUUUUUCAAUU | 1706 | CCGAUAUUCCUCAGGUACUC |
| 1637 | ACUUUCCAUUCUCUGCUGGA | 1707 | GAGUACCUGAGGAAUAUCGG |
| 1638 | ACAAAGUCACAUGGUUCACA | 1708 | GGAGUACCUGAGGAAUAUCG |
| 1639 | GUACAAGAGAUAGAAAGACC | 1709 | CUCAGGUACUCCAAAGAUUC |
| 1640 | CUGGAUGACGUGAGUAAACC | 1710 | AGGUUUACUCACGUCAUCCA |
| 1641 | GUUUAUUUUUGUUCCACAAG | 1711 | ACUCACGUCAUCCAGCAGAG |
| 1642 | CACAAAAUGUAGGGUUAUAA | 1712 | UCUGCUGGAUGACGUGAGUA |
| 1643 | GGGGAAAAUUUAGAAAUAUA | 1713 | CAUUCUCUGCUGGAUGACGU |
| 1644 | CUUGCUUGCUUUUUAAUAUU | 1714 | CCAUUCUCUGCUGGAUGACG |
| 1645 | CUUUGAGUGCUGUCUCCAUG | 1715 | GACUUUCCAUUCUCUGCUGG |
| 1646 | AUAAAGUAAGGCAUGGUUGU | 1716 | AGGAAAUUUGACUUUCCAUU |
| 1647 | GUUAAUCUGGUUUAUUUUUG | 1717 | CCUGAAUUGCUAUGUGUCUG |
| 1648 | AUGUAUCUGAGCAGGUUGCU | 1718 | CUGAAUUGCUAUGUGUCUGG |
| 1649 | CUUAGAAUUUGGGGGAAAAU | 1719 | CUAUGUGUCUGGGUUUCAUC |
| 1650 | GAUUGGAUGAAUUCCAAAUU | 1720 | AAUGUCGGAUGGAUGAAACC |
| 1651 | UGCACAAAAUGUAGGGUUAU | 1721 | CAUCCAUCCGACAUUGAAGU |
| 1652 | GAAAUAUAAUUGACAGGAUU | 1722 | AUCCAUCCGACAUUGAAGUU |
| 1653 | AGUGCUGUCUCCAUGUUUGA | 1723 | AGUAAGUCAACUUCAAUGUC |
| 1654 | GGAGGGCUGGCAACUUAGAG | 1724 | UUCAGUAAGUCAACUUCAAU |
| 1655 | AACUCUUCAAUCUCUUGCAC | 1725 | AAGUUGACUUACUGAAGAAU |
| 1656 | AUAAUGUUAACAUGGACAUG | 1726 | ACUUACUGAAGAAUGGAGAG |
| 1657 | CUUAUACACUUACACUUUAU | 1727 | UCUCUCCAUUCUUCAGUAAG |
| 1658 | AUAUUGAUAUGCUUAUACAC | 1728 | CUGAAGAAUGGAGAGAGAAU |
| 1659 | GGGUUAUAAUAAUGUUAACA | 1729 | AAUUCUCUCUCCAUUCUUCA |
| 1660 | CAUUUGAUAAAGUAAGGCAU | 1730 | CAAUUCUCUCUCCAUUCUUC |
| 1661 | UUUUUGUUCCACAAGUUAAA | 1731 | UCAAUUCUCUCUCCAUUCUU |
| 1662 | UUCCACAAGUUAAAUAAAUC | 1732 | UUCAAUUCUCUCUCCAUUCU |
| 1663 | UCUGAGCAGGUUGCUCCACA | 1733 | AAAAAGUGGAGCAUUCAGAC |
| 1664 | AUUCUACUUUGAGUGCUGUC | 1734 | CUGAAAGACAAGUCUGAAUG |
| 1665 | AGCAGGUUGCUCCACAGGUA | 1735 | AGACUUGUCUUUCAGCAAGG |
| 1666 | AUUGACAGGAUUAUUGGAAA | 1736 | UCUUUCAGCAAGGACUGGUC |
| 1667 | AAGAUGCCGCAUUUGGAUUG | 1737 | CAGCAAGGACUGGUCUUUCU |
| 1668 | AUGAAUGAAACAUUUUGUCA | 1738 | AGCAAGGACUGGUCUUUCUA |
| 1669 | CAUACUCUGCUUAGAAUUUG | 1739 | CUAUCUCUUGUACUACACUG |
| 1670 | UAAUUCUACUUUGAGUGCUG | 1740 | UAUCUCUUGUACUACACUGA |
| 1671 | CACUUACACUUUAUGCACAA | 1741 | AGUGUAGUACAAGAGAUAGA |
| 1672 | ACCAAGAUGUUGAUGUUGGA | 1742 | UACUACACUGAAUUCACCCC |
| 1673 | CAUAAAGUGUAAGUGUAUAA | 1743 | AGUGGGGGUGAAUUCAGUGU |
| 1674 | GAACAAAAAUAAACCAGAUU | 1744 | CAGUGGGGGUGAAUUCAGUG |
| 1675 | CUCCCCACCUCUAAGUUGCC | 1745 | UCAGUGGGGGUGAAUUCAGU |
| 1676 | AGUUGCCAGCCCUCCUAGAG | 1746 | UUCAGUGGGGGUGAAUUCAG |
| 1677 | AAUUGGAAGUUAACUUAUGC | 1747 | ACCCCCACUGAAAAAGAUGA |
| 1678 | AGCAGAGUAUGUAAAUUGGA | 1748 | ACACGGCAGGCAUACUCAUC |
| 1679 | ACAAAUUUCCAAUAAUCCUG | 1749 | GGCUGUGACAAAGUCACAUG |
| 1680 | CACGCUUAACUAUCUUAACA | 1750 | GUCACAGCCCAAGAUAGUUA |
| 1681 | UUUAACUUGUGGAACAAAAA | 1751 | ACUAUCUUGGGCUGUGACAA |
| 1682 | UGAUUUAUUUAACUUGUGGA | 1752 | CCCCACUUAACUAUCUUGGG |
| 1683 | GAGCAACCUGCUCAGAUACA | 1367 | AUAUAAGUGGAGGCGUCGCG |
| 1684 | ACUUGUGGAACAAAAAUAAA | 1368 | GGGCCGAGAUGUCUCGCUCC |
| 1685 | AGUGCAAGAGAUUGAAGAGU | 1369 | UGGCCUGGAGGCUAUCCAGC |
| 1686 | AGUGUAUAAGCAUAUCAAUA | 1370 | AAGUUGACUUACUGAAGAAU |
| 1687 | AUUUAUUUAACUUGUGGAAC | 1371 | AGCAAGGACUGGUCUUUCUA |
| 1688 | UGACAAAAUGUUUCAUUCAU | 1372 | AGUGGGGGUGAAUUCAGUGU |
| 1689 | UGCAUAAAGUGUAAGUGUAU | 1351 | CAGUGGGGGUGAAUUCAGUG |
| 1690 | AAGAAGAUCAUGUCCAUGUU | 1373 | GGCUGUGACAAAGUCACAUG |
| 1691 | AAUUUUCCCCCAAAUUCUAA | 1374 | GUCACAGCCCAAGAUAGUUA |
| 1692 | GAAUUCAUCCAAUCCAAAUG | 1375 | UCACAGCCCAAGAUAGUUAA |
| 1693 | UUUCUAAAUUUUCCCCCAAA | 1355 | CAGUGGGGGUGAAUUCA |
| 1694 | ACCCUACAUUUUGUGCAUAA | 1368 | GGGCCGAGAUGUCUCGCUCC |
| 2657 | GGGCCGAGAUGUCUCGC | 2658 | AGCAAGGACUGGUCUUU |

TABLE 7
Spacer sequences of gRNAs (DNA sequences)
targeting human CIITA

| | Spacer sequence (5′ --> 3′), | | |
|---|---|---|---|
| Name | shown as DNA | Target | SEQ ID NO |
| R4503 C2TA_T1.1 | CTACACAATGCGTTGCCTGG | CIITA | 446 |
| R4504 C2TA_T1.2 | GGGCTCTGACAGGTAGGACC | CIITA | 447 |
| R4505 C2TA_T1.3 | TGTAGGAATCCCAGCCAGGC | CIITA | 448 |
| R4506 C2TA_T1.8 | CCTGGCTCCACGCCCTGCTG | CIITA | 449 |
| R4507 C2TA_T1.9 | GGGAAGCTGAGGGCACGAGG | CIITA | 450 |
| R4508 C2TA_T2.1 | ACAGCGATGCTGACCCCCTG | CIITA | 451 |
| R4509 C2TA_T2.2 | TTAACAGCGATGCTGACCCC | CIITA | 452 |
| R4510 C2TA_T2.3 | TATGACCAGATGGACCTGGC | CIITA | 453 |
| R4511 C2TA_T2.4 | GGGCCCCTAGAAGGTGGCTA | CIITA | 454 |
| R4512 C2TA_T2.5 | TAGGGGCCCCAACTCCATGG | CIITA | 455 |
| R4513 C2TA_T2.6 | AGAAGCTCCAGGTAGCCACC | CIITA | 456 |
| R4514 C2TA_T2.7 | TCCAGCCAGGTCCATCTGGT | CIITA | 457 |
| R4515 C2TA_T2.8 | TTCTCCAGCCAGGTCCATCT | CIITA | 458 |
| R5200 | AGCAGGCTGTTGTGTGACAT | CIITA | 459 |
| R5201 | CATGTCACACAACAGCCTGC | CIITA | 460 |
| R5202 | TGTGACATGGAAGGTGATGA | CIITA | 461 |
| R5203 | ATCACCTTCCATGTCACACA | CIITA | 462 |
| R5204 | GCATAAGCCTCCCTGGTCTC | CIITA | 463 |
| R5205 | CAGGACTCCCAGCTGGAGGG | CIITA | 464 |
| R5206 | CTCAGGCCCTCCAGCTGGGA | CIITA | 465 |
| R5207 | TGCTGGCATCTCCATACTCT | CIITA | 466 |
| R5208 | TGCCCAACTTCTGCTGGCAT | CIITA | 467 |
| R5209 | CTGCCCAACTTCTGCTGGCA | CIITA | 468 |
| R5210 | TCTGCCCAACTTCTGCTGGC | CIITA | 469 |
| R5211 | TGACTTTTCTGCCCAACTTC | CIITA | 470 |
| R5212 | CTGACTTTTCTGCCCAACTT | CIITA | 471 |
| R5213 | TCTGACTTTTCTGCCCAACT | CIITA | 472 |
| R5214 | CCAGAGGAGCTTCCGGCAGA | CIITA | 473 |
| R5215 | AGGTCTGCCGGAAGCTCCTC | CIITA | 474 |
| R5216 | CGGCAGACCTGAAGCACTGG | CIITA | 475 |
| R5217 | CAGTGCTTCAGGTCTGCCGG | CIITA | 476 |
| R5218 | AACAGCGCAGGCAGTGGCAG | CIITA | 477 |
| R5219 | AACCAGGAGCCAGCCTCCGG | CIITA | 478 |
| R5220 | TCCAGGCGCATCTGGCCGGA | CIITA | 479 |
| R5221 | CTCCAGGCGCATCTGGCCGG | CIITA | 480 |
| R5222 | TCTCCAGGCGCATCTGGCCG | CIITA | 481 |
| R5223 | CTCCAGTTCCTCGTTGAGCT | CIITA | 482 |
| R5224 | TCCAGTTCCTCGTTGAGCTG | CIITA | 483 |
| R5225 | AGGCAGCTCAACGAGGAACT | CIITA | 484 |
| R5226 | CTCGTTGAGCTGCCTGAATC | CIITA | 485 |
| R5227 | AGCTGCCTGAATCTCCCTGA | CIITA | 486 |
| R5228 | GTCCCCACCATCTCCACTCT | CIITA | 487 |
| R5229 | TCCCCACCATCTCCACTCTG | CIITA | 488 |
| R5230 | CCAGAGCCCATGGGGCAGAG | CIITA | 489 |
| R5231 | GCCAGAGCCCATGGGGCAGA | CIITA | 490 |
| R5232 | CAGCCTCAGAGATTTGCCAG | CIITA | 491 |
| R5233 | GGAGGCCGTGGACAGTGAAT | CIITA | 492 |
| R5234 | ACTGTCCACGGCCTCCCAAC | CIITA | 493 |
| R5235 | GCTCCATCAGCCACTGACCT | CIITA | 494 |
| R5236 | AGGCATGCTGGGCAGGTCAG | CIITA | 495 |
| R5237 | CTCGGGAGGTCAGGGCAGGT | CIITA | 496 |
| R5238 | GCTCGGGAGGTCAGGGCAGG | CIITA | 497 |
| R5239 | GAGACCTCTCCAGCTGCCGG | CIITA | 498 |
| R5240 | TTGGAGACCTCTCCAGCTGC | CIITA | 499 |
| R5241 | GAAGCTTGTTGGAGACCTCT | CIITA | 500 |
| R5242 | GGAAGCTTGTTGGAGACCTC | CIITA | 501 |
| R5243 | TGGAAGCTTGTTGGAGACCT | CIITA | 502 |
| R5244 | TACCGCTCACTGCAGGACAC | CIITA | 503 |
| R5245 | CTGCTGCTCCTCTCCAGCCT | CIITA | 504 |
| R5246 | CCGCTCCAGGCTCTTGCTGC | CIITA | 505 |
| R5247 | TGCCCAGTCCGGGGTGGCCA | CIITA | 506 |
| R5248 | GGCCAGCTGCCGTTCTGCCC | CIITA | 507 |
| R5249 | GCAGCCAACAGCACCTCAGC | CIITA | 508 |
| R5250 | GCTGCCAAGGAGCACCGGCG | CIITA | 509 |
| R5251 | CCCAGCACAGCAATCACTCG | CIITA | 510 |
| R5252 | GCCCAGCACAGCAATCACTC | CIITA | 511 |
| R5253 | CTGTGCTGGGCAAAGCTGGT | CIITA | 512 |
| R5254 | CCCTGACCAGCTTTGCCCAG | CIITA | 513 |
| R5255 | GGCTGGGGCAGTGAGCCGGG | CIITA | 514 |
| R5256 | TGGCCGGCTTCCCCAGTACG | CIITA | 515 |
| R5257 | CCCAGTACGACTTTGTCTTC | CIITA | 516 |
| R5258 | GTCTTCTCTGTCCCCTGCCA | CIITA | 517 |
| R5259 | TCTTCTCTGTCCCCTGCCAT | CIITA | 518 |
| R5260 | TCTGTCCCCTGCCATTGCTT | CIITA | 519 |
| R5261 | AAGCAATGGCAGGGGACAGA | CIITA | 520 |
| R5262 | CTTGAACCGTCCGGGGGATG | CIITA | 521 |
| R5263 | AACCGTCCGGGGGATGCCTA | CIITA | 522 |
| R5264 | TCCCTGGGCCCACAGCCACT | CIITA | 523 |
| R5265 | AAGATGTGGCTGAAAACCTC | CIITA | 524 |
| R5266 | TCAGCCACATCTTGAAGAGA | CIITA | 525 |
| R5267 | CAGCCACATCTTGAAGAGAC | CIITA | 526 |
| R5268 | AGCCACATCTTGAAGAGACC | CIITA | 527 |
| R5269 | AAGAGACCTGACCGCGTTCT | CIITA | 528 |
| R5270 | TGCTCATCCTAGACGGCTTC | CIITA | 529 |
| R5271 | CAGCTCCTCGAAGCCGTCTA | CIITA | 530 |
| R5272 | CGCTTCCAGCTCCTCGAAGC | CIITA | 531 |
| R5273 | GAGGAGCTGGAAGCGCAAGA | CIITA | 532 |
| R5274 | CTGCACAGCACGTGCGGACC | CIITA | 533 |
| R5275 | TGGAAAAGGCCGGCCAGCAG | CIITA | 534 |
| R5276 | TTCTGGAAAAGGCCGGCCAG | CIITA | 535 |
| R5277 | TCCAGAAGAAGCTGCTCCGA | CIITA | 536 |
| R5278 | CCAGAAGAAGCTGCTCCGAG | CIITA | 537 |
| R5279 | CAGAAGAAGCTGCTCCGAGG | CIITA | 538 |
| R5280 | CACCCTCCTCCTCACAGCCC | CIITA | 539 |
| R5281 | CTCAGGCTCTGGACCAGGCG | CIITA | 540 |
| R5282 | GAGCTGTCCGGCTTCTCCAT | CIITA | 541 |
| R5283 | AGCTGTCCGGCTTCTCCATG | CIITA | 542 |
| R5284 | TCCATGGAGCAGGCCCAGGC | CIITA | 543 |
| R5285 | GAGAGCTCAGGGATGACAGA | CIITA | 544 |
| R5286 | AGAGCTCAGGGATGACAGAG | CIITA | 545 |
| R5287 | GTGCTCTGTCATCCCTGAGC | CIITA | 546 |
| R5288 | TTCTCAGTCACAGCCACAGC | CIITA | 547 |
| R5289 | TCAGTCACAGCCACAGCCCT | CIITA | 548 |
| R5290 | GTGCCGGGCAGTGTGCCAGC | CIITA | 549 |
| R5291 | TGCCGGGCAGTGTGCCAGCT | CIITA | 550 |
| R5292 | GCGTCCTCCCCAAGCTCCAG | CIITA | 551 |
| R5293 | GGGAGGACGCCAAGCTGCCC | CIITA | 552 |
| R5294 | GCCAGCTCTGCCAGGGCCCC | CIITA | 553 |
| R5295 | ATGTCTGCGGCCCAGCTCCC | CIITA | 554 |
| R5392 | GATGTCTGCGGCCCAGCTCC | CIITA | 555 |
| R5393 | CCATCCGCAGACGTGAGGAC | CIITA | 556 |
| R5394 | GCCATCGCCCAGGTCCTCAC | CIITA | 557 |
| R5395 | GGCCATCGCCCAGGTCCTCA | CIITA | 558 |
| R5396 | GACTAAGCCTTTGGCCATCG | CIITA | 559 |
| R5397 | GTCCAACACCCACCGCGGGC | CIITA | 560 |
| R5398 | CAGGAGGAAGCTGGGGAAGG | CIITA | 561 |
| R5399 | CCCAGCTTCCTCCTGCAATG | CIITA | 562 |
| R5400 | CTCCTGCAATGCTTCCTGGG | CIITA | 563 |
| R5401 | CTGGGGGCCCTGTGGCTGGC | CIITA | 564 |
| R5402 | GCCACTCAGAGCCAGCCACA | CIITA | 565 |
| R5403 | CGCCACTCAGAGCCAGCCAC | CIITA | 566 |
| R5404 | ATTTCGCCACTCAGAGCCAG | CIITA | 567 |
| R5405 | TCCTTGATTTCGCCACTCAG | CIITA | 568 |
| R5406 | GGGTCAATGCTAGGTACTGC | CIITA | 569 |
| R5407 | CTTGGGGTCAATGCTAGGTA | CIITA | 570 |
| R5408 | TTCCTTGGGGTCAATGCTAG | CIITA | 571 |
| R5409 | ACCCCAAGGAAGAAGAGGCC | CIITA | 572 |
| R5410 | TCATAGGGCCTCTTCTTCCT | CIITA | 573 |
| R5411 | CTGGCTGGGCTGATCTTCCA | CIITA | 574 |
| R5412 | TGGCTGGGCTGATCTTCCAG | CIITA | 575 |
| R5413 | CAGCCTCCCGCCCGCTGCCT | CIITA | 576 |
| R5414 | CTGTCCACCGAGGCAGCCGC | CIITA | 577 |
| R5415 | TGCTTCCTGTCCACCGAGGC | CIITA | 578 |
| R5416 | AGGTACCTCGCAAGCACCTT | CIITA | 579 |
| R5417 | CGAGGTACCTGAAGCGGCTG | CIITA | 580 |
| R5418 | CAGCCTCCTCGGCCTCGTGG | CIITA | 581 |
| R5419 | GGCAGCACGTGGTACAGGAG | CIITA | 582 |
| R5420 | GCAGCACGTGGTACAGGAGC | CIITA | 583 |
| R5421 | TCTGGGCACCCGCCTCACGC | CIITA | 584 |
| R5422 | CTGGGCACCCGCCTCACGCC | CIITA | 585 |
| R5423 | TGGGCACCCGCCTCACGCCT | CIITA | 586 |
| R5424 | CCCAGTACATGTGCATCAGG | CIITA | 587 |
| R5425 | GCCCGCCGCCTCCAAGGCCT | CIITA | 588 |
| R5426 | GAGGCGGCGGGCCAAGACTT | CIITA | 589 |
| R5427 | TCCCTGGACCTCCGCAGCAC | CIITA | 590 |
| R5428 | GCCCCTCTGGATTGGGGAGC | CIITA | 591 |
| R5429 | CCCCTCTGGATTGGGGAGCC | CIITA | 592 |
| R5430 | GGGAGCCTCGTGGGACTCAG | CIITA | 593 |
| R5431 | GTCTCCCCATGCTGCTGCAG | CIITA | 594 |
| R5432 | TCCTCTGCTGCCTGAAGTAG | CIITA | 595 |
| R5433 | AGGCAGCAGAGGAGAAGTTC | CIITA | 596 |
| R5434 | AAAGGCTCGATGGTGAACTT | CIITA | 597 |
| R5435 | GAAAGGCTCGATGGTGAACT | CIITA | 598 |
| R5436 | ACCATCGAGCCTTTCAAAGC | CIITA | 599 |
| R5437 | GCTTTGAAAGGCTCGATGGT | CIITA | 600 |
| R5438 | AGGGACTTGGCTTTGAAAGG | CIITA | 601 |
| R5439 | CAAAGCCAAGTCCCTGAAGG | CIITA | 602 |
| R5440 | AAAGCCAAGTCCCTGAAGGA | CIITA | 603 |
| R5441 | CACATCCTTCAGGGACTTGG | CIITA | 604 |
| R5442 | CCAGGTCTTCCACATCCTTC | CIITA | 605 |
| R5443 | CCCAGGTCTTCCACATCCTT | CIITA | 606 |
| R5444 | CTCGGAAGACACAGCTGGGG | CIITA | 607 |
| R5445 | GGTCCCGAACAGCAGGGAGC | CIITA | 608 |
| R5446 | AGGTCCCGAACAGCAGGGAG | CIITA | 609 |
| R5447 | TTTAGGTCCCGAACAGCAGG | CIITA | 610 |
| R5448 | CTTTAGGTCCCGAACAGCAG | CIITA | 611 |
| R5449 | GGGACCTAAAGAAACTGGAG | CIITA | 612 |
| R5450 | GGGAAAGCCTGGGGGCCTGA | CIITA | 613 |
| R5451 | GGGGAAAGCCTGGGGGCCTG | CIITA | 614 |
| R5452 | CCCCAAACTGGTGCGGATCC | CIITA | 615 |
| R5453 | CCCAAACTGGTGCGGATCCT | CIITA | 616 |
| R5454 | TTCTCACTCAGCGCATCCAG | CIITA | 617 |
| R5455 | AGCTGGGGGAAGGTGGCTGA | CIITA | 618 |
| R5456 | CCCCAGCTGAAGTCCTTGGA | CIITA | 619 |
| R5457 | CAAGGACTTCAGCTGGGGGA | CIITA | 620 |
| R5458 | CCAAGGACTTCAGCTGGGGG | CIITA | 621 |
| R5459 | AGGGTTTCCAAGGACTTCAG | CIITA | 622 |
| R5460 | TAGGCACCCAGGTCAGTGAT | CIITA | 623 |
| R5461 | GTAGGCACCCAGGTCAGTGA | CIITA | 624 |
| R5462 | GCTCGCTGCATCCCTGCTCA | CIITA | 625 |
| R5463 | GCCTGAGCAGGGATGCAGCG | CIITA | 626 |
| R5464 | TACAATAACTGCATCTGCGA | CIITA | 627 |
| R5465 | GCTCGTGTGCTTCCGGACAT | CIITA | 628 |
| R5466 | CGGACATGGTGTCCCTCCGG | CIITA | 629 |
| R5467 | ACGGCTGCCGGGGCCCAGCA | CIITA | 630 |
| R5468 | GGAGGTGTCCTCATGTGGAG | CIITA | 631 |
| R5469 | CTGGACACTGAATGGGATGG | CIITA | 632 |
| R5470 | AGTGTCCAGGAACACCTGCA | CIITA | 633 |
| R5471 | CAGGTGTTCCTGGACACTGA | CIITA | 634 |
| R5472 | TTGCAGGTGTTCCTGGACAC | CIITA | 635 |
| R5473 | ACGGATCAGCCTGAGATGAT | CIITA | 636 |

TABLE 7.1
Spacer sequences of gRNAs targeting human CIITA

| SEQ | |
|---|---|
| ID | |
| NO | Spacer Sequence |
| 1754 | UGCUUCUGAGCUGGGCAUCC |
| 1755 | AGCUGGGCAUCCGAAGGCAU |
| 1756 | CUUCUGAGCUGGGCAUCCGA |
| 1757 | GGAAUCCCAGCCAGGCAGCA |
| 1758 | UAGGAAUCCCAGCCAGGCAG |
| 1759 | GCAGCCCCUCCUCGUGCCCU |
| 1760 | ACAGGUAGGACCCAGCAGGG |
| 1761 | UGACCAGAUGGACCUGGCUG |
| 1762 | CCACUUCUAUGACCAGAUGG |
| 1763 | ACCAGAUGGACCUGGCUGGA |
| 1764 | CCACCAUGGAGUUGGGGCCC |
| 1765 | CCUCUACCACUUCUAUGACC |
| 1766 | GGGGCCCCAACUCCAUGGUG |
| 1767 | GUCAUAGAAGUGGUAGAGGC |
| 1768 | ACAUGGAAGGUGAUGAAGAG |
| 1769 | UGACAUGGAAGGUGAUGAAG |
| 1770 | UCUUCCAGGACUCCCAGCUG |
| 1771 | CUACACAAUGCGUUGCCUGG |
| 1772 | GGGCUCUGACAGGUAGGACC |
| 1773 | UGUAGGAAUCCCAGCCAGGC |
| 1774 | CCUGGCUCCACGCCCUGCUG |
| 1775 | GGGAAGCUGAGGGCACGAGG |
| 1776 | ACAGCGAUGCUGACCCCCUG |
| 1777 | UUAACAGCGAUGCUGACCCC |
| 1778 | UAUGACCAGAUGGACCUGGC |
| 1779 | GGGCCCCUAGAAGGUGGCUA |
| 1780 | UAGGGGCCCCAACUCCAUGG |
| 1781 | AGAAGCUCCAGGUAGCCACC |
| 1782 | UCCAGCCAGGUCCAUCUGGU |
| 1783 | UUCUCCAGCCAGGUCCAUCU |
| 1784 | AGCAGGCUGUUGUGUGACAU |
| 1785 | CAUGUCACACAACAGCCUGC |
| 1786 | UGUGACAUGGAAGGUGAUGA |
| 1787 | AUCACCUUCCAUGUCACACA |
| 1788 | GCAUAAGCCUCCCUGGUCUC |
| 1789 | CAGGACUCCCAGCUGGAGGG |
| 1790 | CUCAGGCCCUCCAGCUGGGA |
| 1791 | UGCUGGCAUCUCCAUACUCU |
| 1792 | UGCCCAACUUCUGCUGGCAU |
| 1793 | CUGCCCAACUUCUGCUGGCA |
| 1794 | UCUGCCCAACUUCUGCUGGC |
| 1795 | UGACUUUUCUGCCCAACUUC |
| 1796 | CUGACUUUUCUGCCCAACUU |
| 1797 | UCUGACUUUUCUGCCCAACU |
| 1798 | CCAGAGGAGCUUCCGGCAGA |
| 1799 | AGGUCUGCCGGAAGCUCCUC |
| 1800 | CGGCAGACCUGAAGCACUGG |
| 1801 | CAGUGCUUCAGGUCUGCCGG |
| 1802 | AACAGCGCAGGCAGUGGCAG |
| 1803 | AACCAGGAGCCAGCCUCCGG |
| 1804 | UCCAGGCGCAUCUGGCCGGA |
| 1805 | CUCCAGGCGCAUCUGGCCGG |
| 1806 | UCUCCAGGCGCAUCUGGCCG |
| 1807 | CUCCAGUUCCUCGUUGAGCU |
| 1808 | UCCAGUUCCUCGUUGAGCUG |
| 1809 | AGGCAGCUCAACGAGGAACU |
| 1810 | CUCGUUGAGCUGCCUGAAUC |
| 1811 | AGCUGCCUGAAUCUCCCUGA |
| 1812 | GUCCCCACCAUCUCCACUCU |
| 1813 | UCCCCACCAUCUCCACUCUG |
| 1814 | CCAGAGCCCAUGGGGCAGAG |
| 1815 | GCCAGAGCCCAUGGGGCAGA |
| 1816 | CAGCCUCAGAGAUUUGCCAG |
| 1817 | GGAGGCCGUGGACAGUGAAU |
| 1818 | ACUGUCCACGGCCUCCCAAC |
| 1819 | GCUCCAUCAGCCACUGACCU |
| 1820 | AGGCAUGCUGGGCAGGUCAG |
| 1821 | CUCGGGAGGUCAGGGCAGGU |
| 1822 | GCUCGGGAGGUCAGGGCAGG |
| 1823 | GAGACCUCUCCAGCUGCCGG |
| 1824 | UUGGAGACCUCUCCAGCUGC |
| 1825 | GAAGCUUGUUGGAGACCUCU |
| 1826 | GGAAGCUUGUUGGAGACCUC |
| 1827 | UGGAAGCUUGUUGGAGACCU |
| 1828 | UACCGCUCACUGCAGGACAC |
| 1829 | CUGCUGCUCCUCUCCAGCCU |
| 1830 | CCGCUCCAGGCUCUUGCUGC |
| 1831 | UGCCCAGUCCGGGGUGGCCA |
| 1832 | GGCCAGCUGCCGUUCUGCCC |
| 1833 | GCAGCCAACAGCACCUCAGC |
| 1834 | GCUGCCAAGGAGCACCGGCG |
| 1835 | CCCAGCACAGCAAUCACUCG |
| 1836 | GCCCAGCACAGCAAUCACUC |
| 1837 | CUGUGCUGGGCAAAGCUGGU |
| 1838 | CCCUGACCAGCUUUGCCCAG |
| 1839 | GGCUGGGGCAGUGAGCCGGG |
| 1840 | UGGCCGGCUUCCCCAGUACG |
| 1841 | CCCAGUACGACUUUGUCUUC |
| 1842 | GUCUUCUCUGUCCCCUGCCA |
| 1843 | UCUUCUCUGUCCCCUGCCAU |
| 1844 | UCUGUCCCCUGCCAUUGCUU |
| 1845 | AAGCAAUGGCAGGGGACAGA |
| 1846 | CUUGAACCGUCCGGGGGAUG |
| 1847 | AACCGUCCGGGGGAUGCCUA |
| 1848 | UCCCUGGGCCCACAGCCACU |
| 1849 | AAGAUGUGGCUGAAAACCUC |
| 1850 | UCAGCCACAUCUUGAAGAGA |
| 1851 | CAGCCACAUCUUGAAGAGAC |
| 1852 | AGCCACAUCUUGAAGAGACC |
| 1853 | AAGAGACCUGACCGCGUUCU |
| 1854 | UGCUCAUCCUAGACGGCUUC |
| 1855 | CAGCUCCUCGAAGCCGUCUA |
| 1856 | CGCUUCCAGCUCCUCGAAGC |
| 1857 | GAGGAGCUGGAAGCGCAAGA |
| 1858 | CUGCACAGCACGUGCGGACC |
| 1859 | UGGAAAAGGCCGGCCAGCAG |
| 1860 | UUCUGGAAAAGGCCGGCCAG |
| 1861 | UCCAGAAGAAGCUGCUCCGA |
| 1862 | CCAGAAGAAGCUGCUCCGAG |
| 1863 | CAGAAGAAGCUGCUCCGAGG |
| 1864 | CACCCUCCUCCUCACAGCCC |
| 1865 | CUCAGGCUCUGGACCAGGCG |
| 1866 | GAGCUGUCCGGCUUCUCCAU |
| 1867 | AGCUGUCCGGCUUCUCCAUG |
| 1868 | UCCAUGGAGCAGGCCCAGGC |
| 1869 | GAGAGCUCAGGGAUGACAGA |
| 1870 | AGAGCUCAGGGAUGACAGAG |
| 1871 | GUGCUCUGUCAUCCCUGAGC |
| 1872 | UUCUCAGUCACAGCCACAGC |
| 1873 | UCAGUCACAGCCACAGCCCU |
| 1874 | GUGCCGGGCAGUGUGCCAGC |
| 1875 | UGCCGGGCAGUGUGCCAGCU |
| 1876 | GCGUCCUCCCCAAGCUCCAG |
| 1877 | GGGAGGACGCCAAGCUGCCC |
| 1878 | GCCAGCUCUGCCAGGGCCCC |
| 1879 | AUGUCUGCGGCCCAGCUCCC |
| 1880 | GAUGUCUGCGGCCCAGCUCC |
| 1881 | CCAUCCGCAGACGUGAGGAC |
| 1882 | GCCAUCGCCCAGGUCCUCAC |
| 1883 | GGCCAUCGCCCAGGUCCUCA |
| 1884 | GACUAAGCCUUUGGCCAUCG |
| 1885 | GUCCAACACCCACCGCGGGC |
| 1886 | CAGGAGGAAGCUGGGGAAGG |
| 1887 | CCCAGCUUCCUCCUGCAAUG |
| 1888 | CUCCUGCAAUGCUUCCUGGG |
| 1889 | CUGGGGGCCCUGUGGCUGGC |
| 1890 | GCCACUCAGAGCCAGCCACA |
| 1891 | CGCCACUCAGAGCCAGCCAC |
| 1892 | AUUUCGCCACUCAGAGCCAG |
| 1893 | UCCUUGAUUUCGCCACUCAG |
| 1894 | GGGUCAAUGCUAGGUACUGC |
| 1895 | CUUGGGGUCAAUGCUAGGUA |
| 1896 | UUCCUUGGGGUCAAUGCUAG |
| 1897 | ACCCCAAGGAAGAAGAGGCC |
| 1898 | UCAUAGGGCCUCUUCUUCCU |
| 1899 | CUGGCUGGGCUGAUCUUCCA |
| 1900 | UGGCUGGGCUGAUCUUCCAG |
| 1901 | CAGCCUCCCGCCCGCUGCCU |
| 1902 | CUGUCCACCGAGGCAGCCGC |
| 1903 | UGCUUCCUGUCCACCGAGGC |
| 1904 | AGGUACCUCGCAAGCACCUU |
| 1905 | CGAGGUACCUGAAGCGGCUG |
| 1906 | CAGCCUCCUCGGCCUCGUGG |
| 1907 | GGCAGCACGUGGUACAGGAG |
| 1908 | GCAGCACGUGGUACAGGAGC |
| 1909 | UCUGGGCACCCGCCUCACGC |
| 1910 | CUGGGCACCCGCCUCACGCC |
| 1911 | UGGGCACCCGCCUCACGCCU |
| 1912 | CCCAGUACAUGUGCAUCAGG |
| 1913 | GCCCGCCGCCUCCAAGGCCU |
| 1914 | GAGGCGGCGGGCCAAGACUU |
| 1915 | UCCCUGGACCUCCGCAGCAC |
| 1916 | GCCCCUCUGGAUUGGGGAGC |
| 1917 | CCCCUCUGGAUUGGGGAGCC |
| 1918 | GGGAGCCUCGUGGGACUCAG |
| 1919 | GUCUCCCCAUGCUGCUGCAG |
| 1920 | UCCUCUGCUGCCUGAAGUAG |
| 1921 | AGGCAGCAGAGGAGAAGUUC |
| 1922 | AAAGGCUCGAUGGUGAACUU |
| 1923 | GAAAGGCUCGAUGGUGAACU |
| 1924 | ACCAUCGAGCCUUUCAAAGC |
| 1925 | GCUUUGAAAGGCUCGAUGGU |
| 1926 | AGGGACUUGGCUUUGAAAGG |
| 1927 | CAAAGCCAAGUCCCUGAAGG |
| 1928 | AAAGCCAAGUCCCUGAAGGA |
| 1929 | CACAUCCUUCAGGGACUUGG |
| 1930 | CCAGGUCUUCCACAUCCUUC |
| 1931 | CCCAGGUCUUCCACAUCCUU |
| 1932 | CUCGGAAGACACAGCUGGGG |
| 1933 | GGUCCCGAACAGCAGGGAGC |
| 1934 | AGGUCCCGAACAGCAGGGAG |
| 1935 | UUUAGGUCCCGAACAGCAGG |
| 1936 | CUUUAGGUCCCGAACAGCAG |
| 1937 | GGGACCUAAAGAAACUGGAG |
| 1938 | GGGAAAGCCUGGGGGCCUGA |
| 1939 | GGGGAAAGCCUGGGGGCCUG |
| 1940 | CCCCAAACUGGUGCGGAUCC |
| 1941 | CCCAAACUGGUGCGGAUCCU |
| 1942 | UUCUCACUCAGCGCAUCCAG |
| 1943 | AGCUGGGGGAAGGUGGCUGA |
| 1944 | CCCCAGCUGAAGUCCUUGGA |
| 1945 | CAAGGACUUCAGCUGGGGGA |
| 1946 | CCAAGGACUUCAGCUGGGGG |
| 1947 | AGGGUUUCCAAGGACUUCAG |
| 1948 | UAGGCACCCAGGUCAGUGAU |
| 1949 | GUAGGCACCCAGGUCAGUGA |
| 1950 | GCUCGCUGCAUCCCUGCUCA |
| 1951 | GCCUGAGCAGGGAUGCAGCG |
| 1952 | UACAAUAACUGCAUCUGCGA |
| 1953 | GCUCGUGUGCUUCCGGACAU |
| 1954 | CGGACAUGGUGUCCCUCCGG |
| 1955 | ACGGCUGCCGGGGCCCAGCA |
| 1956 | GGAGGUGUCCUCAUGUGGAG |
| 1957 | CUGGACACUGAAUGGGAUGG |
| 1958 | AGUGUCCAGGAACACCUGCA |
| 1959 | CAGGUGUUCCUGGACACUGA |
| 1960 | UUGCAGGUGUUCCUGGACAC |
| 1961 | ACGGAUCAGCCUGAGAUGAU |

TABLE 8, TABLE 9, TABLE 9.1, TABLE 10, TABLE 10.1, TABLE 11, TABLE 11.1, TABLE 12, TABLE 12.1, TABLE 13, TABLE 14, TABLE 14.1, TABLE 15, TABLE 15.1 and TABLE 16 provide illustrative guide sequences that are useful in the compositions, systems and methods described herein.

TABLE 8
CasΦ.12 gRNAs targeting human CIITA

| | Repeat + spacer sequence RNA | |
|---|---|---|
| Name | Sequence (5′ --> 3′) | SEQ ID NO |
| R4503_CasPhi12_ | CUUUCAAGACUAAUAGAUUGCUCCUUACG | 637 |
| C2TA_T1.1 | AGGAGACCUACACAAUGCGUUGCCUGG | |
| R4504_CasPhi12_ | CUUUCAAGACUAAUAGAUUGCUCCUUACG | 638 |
| C2TA_T1.2 | AGGAGACGGGCUCUGACAGGUAGGACC | |
| R4505_CasPhi12_ | CUUUCAAGACUAAUAGAUUGCUCCUUACG | 639 |
| C2TA_T1.3 | AGGAGACUGUAGGAAUCCCAGCCAGGC | |
| R4506_CasPhi12_ | CUUUCAAGACUAAUAGAUUGCUCCUUACG | 640 |
| C2TA_T1.8 | AGGAGACCCUGGCUCCACGCCCUGCUG | |
| R4507_CasPhi12_ | CUUUCAAGACUAAUAGAUUGCUCCUUACG | 641 |
| C2TA_T1.9 | AGGAGACGGGAAGCUGAGGGCACGAGG | |
| R4508_CasPhi12_ | CUUUCAAGACUAAUAGAUUGCUCCUUACG | 642 |
| C2TA_T2.1 | AGGAGACACAGCGAUGCUGACCCCCUG | |
| R4509_CasPhi12_ | CUUUCAAGACUAAUAGAUUGCUCCUUACG | 643 |
| C2TA_T2.2 | AGGAGACUUAACAGCGAUGCUGACCCC | |
| R4510_CasPhi12_ | CUUUCAAGACUAAUAGAUUGCUCCUUACG | 644 |
| C2TA_T2.3 | AGGAGACUAUGACCAGAUGGACCUGGC | |
| R4511_CasPhi12_ | CUUUCAAGACUAAUAGAUUGCUCCUUACG | 645 |
| C2TA_T2.4 | AGGAGACGGGCCCCUAGAAGGUGGCUA | |
| R4512_CasPhi12_ | CUUUCAAGACUAAUAGAUUGCUCCUUACG | 646 |
| C2TA_T2.5 | AGGAGACUAGGGGCCCCAACUCCAUGG | |
| R4513_CasPhi12_ | CUUUCAAGACUAAUAGAUUGCUCCUUACG | 647 |
| C2TA_T2.6 | AGGAGACAGAAGCUCCAGGUAGCCACC | |
| R4514_CasPhi12_ | CUUUCAAGACUAAUAGAUUGCUCCUUACG | 648 |
| C2TA_T2.7 | AGGAGACUCCAGCCAGGUCCAUCUGGU | |
| R4515_CasPhi12_ | CUUUCAAGACUAAUAGAUUGCUCCUUACG | 649 |
| C2TA_T2.8 | AGGAGACUUCUCCAGCCAGGUCCAUCU | |
| R5200_CasPhi12 | CUUUCAAGACUAAUAGAUUGCUCCUUACG | 650 |
| | AGGAGACAGCAGGCUGUUGUGUGACAU | |
| R5201_CasPhi12 | CUUUCAAGACUAAUAGAUUGCUCCUUACG | 651 |
| | AGGAGACCAUGUCACACAACAGCCUGC | |
| R5202_CasPhi12 | CUUUCAAGACUAAUAGAUUGCUCCUUACG | 652 |
| | AGGAGACUGUGACAUGGAAGGUGAUGA | |
| R5203_CasPhi12 | CUUUCAAGACUAAUAGAUUGCUCCUUACG | 653 |
| | AGGAGACAUCACCUUCCAUGUCACACA | |
| R5204_CasPhi12 | CUUUCAAGACUAAUAGAUUGCUCCUUACG | 654 |
| | AGGAGACGCAUAAGCCUCCCUGGUCUC | |
| R5205_CasPhi12 | CUUUCAAGACUAAUAGAUUGCUCCUUACG | 655 |
| | AGGAGACCAGGACUCCCAGCUGGAGGG | |
| R5206_CasPhi12 | CUUUCAAGACUAAUAGAUUGCUCCUUACG | 656 |
| | AGGAGACCUCAGGCCCUCCAGCUGGGA | |
| R5207_CasPhi12 | CUUUCAAGACUAAUAGAUUGCUCCUUACG | 657 |
| | AGGAGACUGCUGGCAUCUCCAUACUCU | |
| R5208_CasPhi12 | CUUUCAAGACUAAUAGAUUGCUCCUUACG | 658 |
| | AGGAGACUGCCCAACUUCUGCUGGCAU | |
| R5209_CasPhi12 | CUUUCAAGACUAAUAGAUUGCUCCUUACG | 659 |
| | AGGAGACCUGCCCAACUUCUGCUGGCA | |
| R5210_CasPhi12 | CUUUCAAGACUAAUAGAUUGCUCCUUACG | 660 |
| | AGGAGACUCUGCCCAACUUCUGCUGGC | |
| R5211_CasPhi12 | CUUUCAAGACUAAUAGAUUGCUCCUUACG | 661 |
| | AGGAGACUGACUUUUCUGCCCAACUUC | |
| R5212_CasPhi12 | CUUUCAAGACUAAUAGAUUGCUCCUUACG | 662 |
| | AGGAGACCUGACUUUUCUGCCCAACUU | |
| R5213_CasPhi12 | CUUUCAAGACUAAUAGAUUGCUCCUUACG | 663 |
| | AGGAGACUCUGACUUUUCUGCCCAACU | |
| R5214_CasPhi12 | CUUUCAAGACUAAUAGAUUGCUCCUUACG | 664 |
| | AGGAGACCCAGAGGAGCUUCCGGCAGA | |
| R5215_CasPhi12 | CUUUCAAGACUAAUAGAUUGCUCCUUACG | 665 |
| | AGGAGACAGGUCUGCCGGAAGCUCCUC | |
| R5216_CasPhi12 | CUUUCAAGACUAAUAGAUUGCUCCUUACG | 666 |
| | AGGAGACCGGCAGACCUGAAGCACUGG | |
| R5217_CasPhi12 | CUUUCAAGACUAAUAGAUUGCUCCUUACG | 667 |
| | AGGAGACCAGUGCUUCAGGUCUGCCGG | |
| R5218_CasPhi12 | CUUUCAAGACUAAUAGAUUGCUCCUUACG | 668 |
| | AGGAGACAACAGCGCAGGCAGUGGCAG | |
| R5219_CasPhi12 | CUUUCAAGACUAAUAGAUUGCUCCUUACG | 669 |
| | AGGAGACAACCAGGAGCCAGCCUCCGG | |
| R5220_CasPhi12 | CUUUCAAGACUAAUAGAUUGCUCCUUACG | 670 |
| | AGGAGACUCCAGGCGCAUCUGGCCGGA | |
| R5221_CasPhi12 | CUUUCAAGACUAAUAGAUUGCUCCUUACG | 671 |
| | AGGAGACCUCCAGGCGCAUCUGGCCGG | |
| R5222_CasPhi12 | CUUUCAAGACUAAUAGAUUGCUCCUUACG | 672 |
| | AGGAGACUCUCCAGGCGCAUCUGGCCG | |
| R5223_CasPhi12 | CUUUCAAGACUAAUAGAUUGCUCCUUACG | 673 |
| | AGGAGACCUCCAGUUCCUCGUUGAGCU | |
| R5224_CasPhi12 | CUUUCAAGACUAAUAGAUUGCUCCUUACG | 674 |
| | AGGAGACUCCAGUUCCUCGUUGAGCUG | |
| R5225_CasPhi12 | CUUUCAAGACUAAUAGAUUGCUCCUUACG | 675 |
| | AGGAGACAGGCAGCUCAACGAGGAACU | |
| R5226_CasPhi12 | CUUUCAAGACUAAUAGAUUGCUCCUUACG | 676 |
| | AGGAGACCUCGUUGAGCUGCCUGAAUC | |
| R5227_CasPhi12 | CUUUCAAGACUAAUAGAUUGCUCCUUACG | 677 |
| | AGGAGACAGCUGCCUGAAUCUCCCUGA | |
| R5228_CasPhi12 | CUUUCAAGACUAAUAGAUUGCUCCUUACG | 678 |
| | AGGAGACGUCCCCACCAUCUCCACUCU | |
| R5229_CasPhi12 | CUUUCAAGACUAAUAGAUUGCUCCUUACG | 679 |
| | AGGAGACUCCCCACCAUCUCCACUCUG | |
| R5230_CasPhi12 | CUUUCAAGACUAAUAGAUUGCUCCUUACG | 680 |
| | AGGAGACCCAGAGCCCAUGGGGCAGAG | |
| R5231_CasPhi12 | CUUUCAAGACUAAUAGAUUGCUCCUUACG | 681 |
| | AGGAGACGCCAGAGCCCAUGGGGCAGA | |
| R5232_CasPhi12 | CUUUCAAGACUAAUAGAUUGCUCCUUACG | 682 |
| | AGGAGACCAGCCUCAGAGAUUUGCCAG | |
| R5233_CasPhi12 | CUUUCAAGACUAAUAGAUUGCUCCUUACG | 683 |
| | AGGAGACGGAGGCCGUGGACAGUGAAU | |
| R5234_CasPhi12 | CUUUCAAGACUAAUAGAUUGCUCCUUACG | 684 |
| | AGGAGACACUGUCCACGGCCUCCCAAC | |
| R5235_CasPhi12 | CUUUCAAGACUAAUAGAUUGCUCCUUACG | 685 |
| | AGGAGACGCUCCAUCAGCCACUGACCU | |
| R5236_CasPhi12 | CUUUCAAGACUAAUAGAUUGCUCCUUACG | 686 |
| | AGGAGACAGGCAUGCUGGGCAGGUCAG | |
| R5237_CasPhi12 | CUUUCAAGACUAAUAGAUUGCUCCUUACG | 687 |
| | AGGAGACCUCGGGAGGUCAGGGCAGGU | |
| R5238_CasPhi12 | CUUUCAAGACUAAUAGAUUGCUCCUUACG | 688 |
| | AGGAGACGCUCGGGAGGUCAGGGCAGG | |
| R5239_CasPhi12 | CUUUCAAGACUAAUAGAUUGCUCCUUACG | 689 |
| | AGGAGACGAGACCUCUCCAGCUGCCGG | |
| R5240_CasPhi12 | CUUUCAAGACUAAUAGAUUGCUCCUUACG | 690 |
| | AGGAGACUUGGAGACCUCUCCAGCUGC | |
| R5241_CasPhi12 | CUUUCAAGACUAAUAGAUUGCUCCUUACG | 691 |
| | AGGAGACGAAGCUUGUUGGAGACCUCU | |
| R5242_CasPhi12 | CUUUCAAGACUAAUAGAUUGCUCCUUACG | 692 |
| | AGGAGACGGAAGCUUGUUGGAGACCUC | |
| R5243_CasPhi12 | CUUUCAAGACUAAUAGAUUGCUCCUUACG | 693 |
| | AGGAGACUGGAAGCUUGUUGGAGACCU | |
| R5244_CasPhi12 | CUUUCAAGACUAAUAGAUUGCUCCUUACG | 694 |
| | AGGAGACUACCGCUCACUGCAGGACAC | |
| R5245_CasPhi12 | CUUUCAAGACUAAUAGAUUGCUCCUUACG | 695 |
| | AGGAGACCUGCUGCUCCUCUCCAGCCU | |
| R5246_CasPhi12 | CUUUCAAGACUAAUAGAUUGCUCCUUACG | 696 |
| | AGGAGACCCGCUCCAGGCUCUUGCUGC | |
| R5247_CasPhi12 | CUUUCAAGACUAAUAGAUUGCUCCUUACG | 697 |
| | AGGAGACUGCCCAGUCCGGGGUGGCCA | |
| R5248_CasPhi12 | CUUUCAAGACUAAUAGAUUGCUCCUUACG | 698 |
| | AGGAGACGGCCAGCUGCCGUUCUGCCC | |
| R5249_CasPhi12 | CUUUCAAGACUAAUAGAUUGCUCCUUACG | 699 |
| | AGGAGACGCAGCCAACAGCACCUCAGC | |
| R5250_CasPhi12 | CUUUCAAGACUAAUAGAUUGCUCCUUACG | 700 |
| | AGGAGACGCUGCCAAGGAGCACCGGCG | |
| R5251_CasPhi12 | CUUUCAAGACUAAUAGAUUGCUCCUUACG | 701 |
| | AGGAGACCCCAGCACAGCAAUCACUCG | |
| R5252_CasPhi12 | CUUUCAAGACUAAUAGAUUGCUCCUUACG | 702 |
| | AGGAGACGCCCAGCACAGCAAUCACUC | |
| R5253_CasPhi12 | CUUUCAAGACUAAUAGAUUGCUCCUUACG | 703 |
| | AGGAGACCUGUGCUGGGCAAAGCUGGU | |
| R5254_CasPhi12 | CUUUCAAGACUAAUAGAUUGCUCCUUACG | 704 |
| | AGGAGACCCCUGACCAGCUUUGCCCAG | |
| R5255_CasPhi12 | CUUUCAAGACUAAUAGAUUGCUCCUUACG | 705 |
| | AGGAGACGGCUGGGGCAGUGAGCCGGG | |
| R5256_CasPhi12 | CUUUCAAGACUAAUAGAUUGCUCCUUACG | 706 |
| | AGGAGACUGGCCGGCUUCCCCAGUACG | |
| R5257_CasPhi12 | CUUUCAAGACUAAUAGAUUGCUCCUUACG | 707 |
| | AGGAGACCCCAGUACGACUUUGUCUUC | |
| R5258_CasPhi12 | CUUUCAAGACUAAUAGAUUGCUCCUUACG | 708 |
| | AGGAGACGUCUUCUCUGUCCCCUGCCA | |
| R5259_CasPhi12 | CUUUCAAGACUAAUAGAUUGCUCCUUACG | 709 |
| | AGGAGACUCUUCUCUGUCCCCUGCCAU | |
| R5260_CasPhi12 | CUUUCAAGACUAAUAGAUUGCUCCUUACG | 710 |
| | AGGAGACUCUGUCCCCUGCCAUUGCUU | |
| R5261_CasPhi12 | CUUUCAAGACUAAUAGAUUGCUCCUUACG | 711 |
| | AGGAGACAAGCAAUGGCAGGGGACAGA | |
| R5262_CasPhi12 | CUUUCAAGACUAAUAGAUUGCUCCUUACG | 712 |
| | AGGAGACCUUGAACCGUCCGGGGGAUG | |
| R5263_CasPhi12 | CUUUCAAGACUAAUAGAUUGCUCCUUACG | 713 |
| | AGGAGACAACCGUCCGGGGGAUGCCUA | |
| R5264_CasPhi12 | CUUUCAAGACUAAUAGAUUGCUCCUUACG | 714 |
| | AGGAGACUCCCUGGGCCCACAGCCACU | |
| R5265_CasPhi12 | CUUUCAAGACUAAUAGAUUGCUCCUUACG | 715 |
| | AGGAGACAAGAUGUGGCUGAAAACCUC | |
| R5266_CasPhi12 | CUUUCAAGACUAAUAGAUUGCUCCUUACG | 716 |
| | AGGAGACUCAGCCACAUCUUGAAGAGA | |
| R5267_CasPhi12 | CUUUCAAGACUAAUAGAUUGCUCCUUACG | 717 |
| | AGGAGACCAGCCACAUCUUGAAGAGAC | |
| R5268_CasPhi12 | CUUUCAAGACUAAUAGAUUGCUCCUUACG | 718 |
| | AGGAGACAGCCACAUCUUGAAGAGACC | |
| R5269_CasPhi12 | CUUUCAAGACUAAUAGAUUGCUCCUUACG | 719 |
| | AGGAGACAAGAGACCUGACCGCGUUCU | |
| R5270_CasPhi12 | CUUUCAAGACUAAUAGAUUGCUCCUUACG | 720 |
| | AGGAGACUGCUCAUCCUAGACGGCUUC | |
| R5271_CasPhi12 | CUUUCAAGACUAAUAGAUUGCUCCUUACG | 721 |
| | AGGAGACCAGCUCCUCGAAGCCGUCUA | |
| R5272_CasPhi12 | CUUUCAAGACUAAUAGAUUGCUCCUUACG | 722 |
| | AGGAGACCGCUUCCAGCUCCUCGAAGC | |
| R5273_CasPhi12 | CUUUCAAGACUAAUAGAUUGCUCCUUACG | 723 |
| | AGGAGACGAGGAGCUGGAAGCGCAAGA | |
| R5274_CasPhi12 | CUUUCAAGACUAAUAGAUUGCUCCUUACG | 724 |
| | AGGAGACCUGCACAGCACGUGCGGACC | |
| R5275_CasPhi12 | CUUUCAAGACUAAUAGAUUGCUCCUUACG | 725 |
| | AGGAGACUGGAAAAGGCCGGCCAGCAG | |
| R5276_CasPhi12 | CUUUCAAGACUAAUAGAUUGCUCCUUACG | 726 |
| | AGGAGACUUCUGGAAAAGGCCGGCCAG | |
| R5277_CasPhi12 | CUUUCAAGACUAAUAGAUUGCUCCUUACG | 727 |
| | AGGAGACUCCAGAAGAAGCUGCUCCGA | |
| R5278_CasPhi12 | CUUUCAAGACUAAUAGAUUGCUCCUUACG | 728 |
| | AGGAGACCCAGAAGAAGCUGCUCCGAG | |
| R5279_CasPhi12 | CUUUCAAGACUAAUAGAUUGCUCCUUACG | 729 |
| | AGGAGACCAGAAGAAGCUGCUCCGAGG | |
| R5280_CasPhi12 | CUUUCAAGACUAAUAGAUUGCUCCUUACG | 730 |
| | AGGAGACCACCCUCCUCCUCACAGCCC | |
| R5281_CasPhi12 | CUUUCAAGACUAAUAGAUUGCUCCUUACG | 731 |
| | AGGAGACCUCAGGCUCUGGACCAGGCG | |
| R5282_CasPhi12 | CUUUCAAGACUAAUAGAUUGCUCCUUACG | 732 |
| | AGGAGACGAGCUGUCCGGCUUCUCCAU | |
| R5283_CasPhi12 | CUUUCAAGACUAAUAGAUUGCUCCUUACG | 733 |
| | AGGAGACAGCUGUCCGGCUUCUCCAUG | |
| R5284_CasPhi12 | CUUUCAAGACUAAUAGAUUGCUCCUUACG | 734 |
| | AGGAGACUCCAUGGAGCAGGCCCAGGC | |
| R5285_CasPhi12 | CUUUCAAGACUAAUAGAUUGCUCCUUACG | 735 |
| | AGGAGACGAGAGCUCAGGGAUGACAGA | |
| R5286_CasPhi12 | CUUUCAAGACUAAUAGAUUGCUCCUUACG | 736 |
| | AGGAGACAGAGCUCAGGGAUGACAGAG | |
| R5287_CasPhi12 | CUUUCAAGACUAAUAGAUUGCUCCUUACG | 737 |
| | AGGAGACGUGCUCUGUCAUCCCUGAGC | |
| R5288_CasPhi12 | CUUUCAAGACUAAUAGAUUGCUCCUUACG | 738 |
| | AGGAGACUUCUCAGUCACAGCCACAGC | |
| R5289_CasPhi12 | CUUUCAAGACUAAUAGAUUGCUCCUUACG | 739 |
| | AGGAGACUCAGUCACAGCCACAGCCCU | |
| R5290_CasPhi12 | CUUUCAAGACUAAUAGAUUGCUCCUUACG | 740 |
| | AGGAGACGUGCCGGGCAGUGUGCCAGC | |
| R5291_CasPhi12 | CUUUCAAGACUAAUAGAUUGCUCCUUACG | 741 |
| | AGGAGACUGCCGGGCAGUGUGCCAGCU | |
| R5292_CasPhi12 | CUUUCAAGACUAAUAGAUUGCUCCUUACG | 742 |
| | AGGAGACGCGUCCUCCCCAAGCUCCAG | |
| R5293_CasPhi12 | CUUUCAAGACUAAUAGAUUGCUCCUUACG | 743 |
| | AGGAGACGGGAGGACGCCAAGCUGCCC | |
| R5294_CasPhi12 | CUUUCAAGACUAAUAGAUUGCUCCUUACG | 744 |
| | AGGAGACGCCAGCUCUGCCAGGGCCCC | |
| R5295_CasPhi12 | CUUUCAAGACUAAUAGAUUGCUCCUUACG | 745 |
| | AGGAGACAUGUCUGCGGCCCAGCUCCC | |
| R5392_CasPhi12 | CUUUCAAGACUAAUAGAUUGCUCCUUACG | 746 |
| | AGGAGACGAUGUCUGCGGCCCAGCUCC | |
| R5393_CasPhi12 | CUUUCAAGACUAAUAGAUUGCUCCUUACG | 747 |
| | AGGAGACCCAUCCGCAGACGUGAGGAC | |
| R5394_CasPhi12 | CUUUCAAGACUAAUAGAUUGCUCCUUACG | 748 |
| | AGGAGACGCCAUCGCCCAGGUCCUCAC | |
| R5395_CasPhi12 | CUUUCAAGACUAAUAGAUUGCUCCUUACG | 749 |
| | AGGAGACGGCCAUCGCCCAGGUCCUCA | |
| R5396_CasPhi12 | CUUUCAAGACUAAUAGAUUGCUCCUUACG | 750 |
| | AGGAGACGACUAAGCCUUUGGCCAUCG | |
| R5397_CasPhi12 | CUUUCAAGACUAAUAGAUUGCUCCUUACG | 751 |
| | AGGAGACGUCCAACACCCACCGCGGGC | |
| R5398_CasPhi12 | CUUUCAAGACUAAUAGAUUGCUCCUUACG | 752 |
| | AGGAGACCAGGAGGAAGCUGGGGAAGG | |
| R5399_CasPhi12 | CUUUCAAGACUAAUAGAUUGCUCCUUACG | 753 |
| | AGGAGACCCCAGCUUCCUCCUGCAAUG | |
| R5400_CasPhi12 | CUUUCAAGACUAAUAGAUUGCUCCUUACG | 754 |
| | AGGAGACCUCCUGCAAUGCUUCCUGGG | |
| R5401_CasPhi12 | CUUUCAAGACUAAUAGAUUGCUCCUUACG | 755 |
| | AGGAGACCUGGGGGCCCUGUGGCUGGC | |
| R5402_CasPhi12 | CUUUCAAGACUAAUAGAUUGCUCCUUACG | 756 |
| | AGGAGACGCCACUCAGAGCCAGCCACA | |
| R5403_CasPhi12 | CUUUCAAGACUAAUAGAUUGCUCCUUACG | 757 |
| | AGGAGACCGCCACUCAGAGCCAGCCAC | |
| R5404_CasPhi12 | CUUUCAAGACUAAUAGAUUGCUCCUUACG | 758 |
| | AGGAGACAUUUCGCCACUCAGAGCCAG | |
| R5405_CasPhi12 | CUUUCAAGACUAAUAGAUUGCUCCUUACG | 759 |
| | AGGAGACUCCUUGAUUUCGCCACUCAG | |
| R5406_CasPhi12 | CUUUCAAGACUAAUAGAUUGCUCCUUACG | 760 |
| | AGGAGACGGGUCAAUGCUAGGUACUGC | |
| R5407_CasPhi12 | CUUUCAAGACUAAUAGAUUGCUCCUUACG | 761 |
| | AGGAGACCUUGGGGUCAAUGCUAGGUA | |
| R5408_CasPhi12 | CUUUCAAGACUAAUAGAUUGCUCCUUACG | 762 |
| | AGGAGACUUCCUUGGGGUCAAUGCUAG | |
| R5409_CasPhi12 | CUUUCAAGACUAAUAGAUUGCUCCUUACG | 763 |
| | AGGAGACACCCCAAGGAAGAAGAGGCC | |
| R5410_CasPhi12 | CUUUCAAGACUAAUAGAUUGCUCCUUACG | 764 |
| | AGGAGACUCAUAGGGCCUCUUCUUCCU | |
| R5411_CasPhi12 | CUUUCAAGACUAAUAGAUUGCUCCUUACG | 765 |
| | AGGAGACCUGGCUGGGCUGAUCUUCCA | |
| R5412_CasPhi12 | CUUUCAAGACUAAUAGAUUGCUCCUUACG | 766 |
| | AGGAGACUGGCUGGGCUGAUCUUCCAG | |
| R5413_CasPhi12 | CUUUCAAGACUAAUAGAUUGCUCCUUACG | 767 |
| | AGGAGACCAGCCUCCCGCCCGCUGCCU | |
| R5414_CasPhi12 | CUUUCAAGACUAAUAGAUUGCUCCUUACG | 768 |
| | AGGAGACCUGUCCACCGAGGCAGCCGC | |
| R5415_CasPhi12 | CUUUCAAGACUAAUAGAUUGCUCCUUACG | 769 |
| | AGGAGACUGCUUCCUGUCCACCGAGGC | |
| R5416_CasPhi12 | CUUUCAAGACUAAUAGAUUGCUCCUUACG | 770 |
| | AGGAGACAGGUACCUCGCAAGCACCUU | |
| R5417_CasPhi12 | CUUUCAAGACUAAUAGAUUGCUCCUUACG | 771 |
| | AGGAGACCGAGGUACCUGAAGCGGCUG | |
| R5418_CasPhi12 | CUUUCAAGACUAAUAGAUUGCUCCUUACG | 772 |
| | AGGAGACCAGCCUCCUCGGCCUCGUGG | |
| R5419_CasPhi12 | CUUUCAAGACUAAUAGAUUGCUCCUUACG | 773 |
| | AGGAGACGGCAGCACGUGGUACAGGAG | |
| R5420_CasPhi12 | CUUUCAAGACUAAUAGAUUGCUCCUUACG | 774 |
| | AGGAGACGCAGCACGUGGUACAGGAGC | |
| R5421_CasPhi12 | CUUUCAAGACUAAUAGAUUGCUCCUUACG | 775 |
| | AGGAGACUCUGGGCACCCGCCUCACGC | |
| R5422_CasPhi12 | CUUUCAAGACUAAUAGAUUGCUCCUUACG | 776 |
| | AGGAGACCUGGGCACCCGCCUCACGCC | |
| R5423_CasPhi12 | CUUUCAAGACUAAUAGAUUGCUCCUUACG | 777 |
| | AGGAGACUGGGCACCCGCCUCACGCCU | |
| R5424_CasPhi12 | CUUUCAAGACUAAUAGAUUGCUCCUUACG | 778 |
| | AGGAGACCCCAGUACAUGUGCAUCAGG | |
| R5425_CasPhi12 | CUUUCAAGACUAAUAGAUUGCUCCUUACG | 779 |
| | AGGAGACGCCCGCCGCCUCCAAGGCCU | |
| R5426_CasPhi12 | CUUUCAAGACUAAUAGAUUGCUCCUUACG | 780 |
| | AGGAGACGAGGCGGCGGGCCAAGACUU | |
| R5427_CasPhi12 | CUUUCAAGACUAAUAGAUUGCUCCUUACG | 781 |
| | AGGAGACUCCCUGGACCUCCGCAGCAC | |
| R5428_CasPhi12 | CUUUCAAGACUAAUAGAUUGCUCCUUACG | 782 |
| | AGGAGACGCCCCUCUGGAUUGGGGAGC | |
| R5429_CasPhi12 | CUUUCAAGACUAAUAGAUUGCUCCUUACG | 783 |
| | AGGAGACCCCCUCUGGAUUGGGGAGCC | |
| R5430_CasPhi12 | CUUUCAAGACUAAUAGAUUGCUCCUUACG | 784 |
| | AGGAGACGGGAGCCUCGUGGGACUCAG | |
| R5431_CasPhi12 | CUUUCAAGACUAAUAGAUUGCUCCUUACG | 785 |
| | AGGAGACGUCUCCCCAUGCUGCUGCAG | |
| R5432_CasPhi12 | CUUUCAAGACUAAUAGAUUGCUCCUUACG | 786 |
| | AGGAGACUCCUCUGCUGCCUGAAGUAG | |
| R5433_CasPhi12 | CUUUCAAGACUAAUAGAUUGCUCCUUACG | 787 |
| | AGGAGACAGGCAGCAGAGGAGAAGUUC | |
| R5434_CasPhi12 | CUUUCAAGACUAAUAGAUUGCUCCUUACG | 788 |
| | AGGAGACAAAGGCUCGAUGGUGAACUU | |
| R5435_CasPhi12 | CUUUCAAGACUAAUAGAUUGCUCCUUACG | 789 |
| | AGGAGACGAAAGGCUCGAUGGUGAACU | |
| R5436_CasPhi12 | CUUUCAAGACUAAUAGAUUGCUCCUUACG | 790 |
| | AGGAGACACCAUCGAGCCUUUCAAAGC | |
| R5437_CasPhi12 | CUUUCAAGACUAAUAGAUUGCUCCUUACG | 791 |
| | AGGAGACGCUUUGAAAGGCUCGAUGGU | |
| R5438_CasPhi12 | CUUUCAAGACUAAUAGAUUGCUCCUUACG | 792 |
| | AGGAGACAGGGACUUGGCUUUGAAAGG | |
| R5439_CasPhi12 | CUUUCAAGACUAAUAGAUUGCUCCUUACG | 793 |
| | AGGAGACCAAAGCCAAGUCCCUGAAGG | |
| R5440_CasPhi12 | CUUUCAAGACUAAUAGAUUGCUCCUUACG | 794 |
| | AGGAGACAAAGCCAAGUCCCUGAAGGA | |
| R5441_CasPhi12 | CUUUCAAGACUAAUAGAUUGCUCCUUACG | 795 |
| | AGGAGACCACAUCCUUCAGGGACUUGG | |
| R5442_CasPhi12 | CUUUCAAGACUAAUAGAUUGCUCCUUACG | 796 |
| | AGGAGACCCAGGUCUUCCACAUCCUUC | |
| R5443_CasPhi12 | CUUUCAAGACUAAUAGAUUGCUCCUUACG | 797 |
| | AGGAGACCCCAGGUCUUCCACAUCCUU | |
| R5444_CasPhi12 | CUUUCAAGACUAAUAGAUUGCUCCUUACG | 798 |
| | AGGAGACCUCGGAAGACACAGCUGGGG | |
| R5445_CasPhi12 | CUUUCAAGACUAAUAGAUUGCUCCUUACG | 799 |
| | AGGAGACGGUCCCGAACAGCAGGGAGC | |
| R5446_CasPhi12 | CUUUCAAGACUAAUAGAUUGCUCCUUACG | 780 |
| | AGGAGACAGGUCCCGAACAGCAGGGAG | |
| R5447_CasPhi12 | CUUUCAAGACUAAUAGAUUGCUCCUUACG | 781 |
| | AGGAGACUUUAGGUCCCGAACAGCAGG | |
| R5448_CasPhi12 | CUUUCAAGACUAAUAGAUUGCUCCUUACG | 782 |
| | AGGAGACCUUUAGGUCCCGAACAGCAG | |
| R5449_CasPhi12 | CUUUCAAGACUAAUAGAUUGCUCCUUACG | 783 |
| | AGGAGACGGGACCUAAAGAAACUGGAG | |
| R5450_CasPhi12 | CUUUCAAGACUAAUAGAUUGCUCCUUACG | 784 |
| | AGGAGACGGGAAAGCCUGGGGGCCUGA | |
| R5451_CasPhi12 | CUUUCAAGACUAAUAGAUUGCUCCUUACG | 785 |
| | AGGAGACGGGGAAAGCCUGGGGGCCUG | |
| R5452_CasPhi12 | CUUUCAAGACUAAUAGAUUGCUCCUUACG | 786 |
| | AGGAGACCCCCAAACUGGUGCGGAUCC | |
| R5453_CasPhi12 | CUUUCAAGACUAAUAGAUUGCUCCUUACG | 787 |
| | AGGAGACCCCAAACUGGUGCGGAUCCU | |
| R5454_CasPhi12 | CUUUCAAGACUAAUAGAUUGCUCCUUACG | 788 |
| | AGGAGACUUCUCACUCAGCGCAUCCAG | |
| R5455_CasPhi12 | CUUUCAAGACUAAUAGAUUGCUCCUUACG | 789 |
| | AGGAGACAGCUGGGGGAAGGUGGCUGA | |
| R5456_CasPhi12 | CUUUCAAGACUAAUAGAUUGCUCCUUACG | 790 |
| | AGGAGACCCCCAGCUGAAGUCCUUGGA | |
| R5457_CasPhi12 | CUUUCAAGACUAAUAGAUUGCUCCUUACG | 791 |
| | AGGAGACCAAGGACUUCAGCUGGGGGA | |
| R5458_CasPhi12 | CUUUCAAGACUAAUAGAUUGCUCCUUACG | 792 |
| | AGGAGACCCAAGGACUUCAGCUGGGGG | |
| R5459_CasPhi12 | CUUUCAAGACUAAUAGAUUGCUCCUUACG | 793 |
| | AGGAGACAGGGUUUCCAAGGACUUCAG | |
| R5460_CasPhi12 | CUUUCAAGACUAAUAGAUUGCUCCUUACG | 794 |
| | AGGAGACUAGGCACCCAGGUCAGUGAU | |
| R5461_CasPhi12 | CUUUCAAGACUAAUAGAUUGCUCCUUACG | 795 |
| | AGGAGACGUAGGCACCCAGGUCAGUGA | |
| R5462_CasPhi12 | CUUUCAAGACUAAUAGAUUGCUCCUUACG | 796 |
| | AGGAGACGCUCGCUGCAUCCCUGCUCA | |
| R5463_CasPhi12 | CUUUCAAGACUAAUAGAUUGCUCCUUACG | 797 |
| | AGGAGACGCCUGAGCAGGGAUGCAGCG | |
| R5464_CasPhi12 | CUUUCAAGACUAAUAGAUUGCUCCUUACG | 798 |
| | AGGAGACUACAAUAACUGCAUCUGCGA | |
| R5465_CasPhi12 | CUUUCAAGACUAAUAGAUUGCUCCUUACG | 799 |
| | AGGAGACGCUCGUGUGCUUCCGGACAU | |
| R5466_CasPhi12 | CUUUCAAGACUAAUAGAUUGCUCCUUACG | 800 |
| | AGGAGACCGGACAUGGUGUCCCUCCGG | |
| R5467_CasPhi12 | CUUUCAAGACUAAUAGAUUGCUCCUUACG | 801 |
| | AGGAGACACGGCUGCCGGGGCCCAGCA | |
| R5468_CasPhi12 | CUUUCAAGACUAAUAGAUUGCUCCUUACG | 802 |
| | AGGAGACGGAGGUGUCCUCAUGUGGAG | |
| R5469_CasPhi12 | CUUUCAAGACUAAUAGAUUGCUCCUUACG | 803 |
| | AGGAGACCUGGACACUGAAUGGGAUGG | |
| R5470_CasPhi12 | CUUUCAAGACUAAUAGAUUGCUCCUUACG | 804 |
| | AGGAGACAGUGUCCAGGAACACCUGCA | |
| R5471_CasPhi12 | CUUUCAAGACUAAUAGAUUGCUCCUUACG | 805 |
| | AGGAGACCAGGUGUUCCUGGACACUGA | |
| R5472_CasPhi12 | CUUUCAAGACUAAUAGAUUGCUCCUUACG | 806 |
| | AGGAGACUUGCAGGUGUUCCUGGACAC | |
| R5473_CasPhi12 | CUUUCAAGACUAAUAGAUUGCUCCUUACG | 807 |
| | AGGAGACACGGAUCAGCCUGAGAUGAU | |

TABLE 9
CasΦ.12 gRNAs (DNA sequences) targeting human TRAC in T cells

| | Repeat + spacer RNA Sequence | SEQ |
|---|---|---|
| Name | (5′ --> 3′), shown as DNA | ID NO |
| R3040_CasPhi12 | CTTTCAAGACTAATAGATTGCTCCTTACGAGGAGACT | 808 |
| | GGATATCTGTGGGACAAGA | |
| R3041_CasPhi12 | CTTTCAAGACTAATAGATTGCTCCTTACGAGGAGACTC | 809 |
| | CCACAGATATCCAGAACC | |
| R3042_CasPhi12 | CTTTCAAGACTAATAGATTGCTCCTTACGAGGAGACG | 810 |
| | AGTCTCTCAGCTGGTACAC | |
| R3043_CasPhi12 | CTTTCAAGACTAATAGATTGCTCCTTACGAGGAGACA | 811 |
| | GAGTCTCTCAGCTGGTACA | |
| R3044_CasPhi12 | CTTTCAAGACTAATAGATTGCTCCTTACGAGGAGACTC | 812 |
| | ACTGGATTTAGAGTCTCT | |
| R3045_CasPhi12 | CTTTCAAGACTAATAGATTGCTCCTTACGAGGAGACA | 813 |
| | GAATCAAAATCGGTGAATA | |
| R3046_CasPhi12 | CTTTCAAGACTAATAGATTGCTCCTTACGAGGAGACG | 814 |
| | AGAATCAAAATCGGTGAAT | |
| R3047_CasPhi12 | CTTTCAAGACTAATAGATTGCTCCTTACGAGGAGACA | 815 |
| | CCGATTTTGATTCTCAAAC | |
| R3048_CasPhi12 | CTTTCAAGACTAATAGATTGCTCCTTACGAGGAGACTT | 816 |
| | TGAGAATCAAAATCGGTG | |
| R3049_CasPhi12 | CTTTCAAGACTAATAGATTGCTCCTTACGAGGAGACG | 817 |
| | TTTGAGAATCAAAATCGGT | |
| R3050_CasPhi12 | CTTTCAAGACTAATAGATTGCTCCTTACGAGGAGACT | 818 |
| | GATTCTCAAACAAATGTGT | |
| R3051_CasPhi12 | CTTTCAAGACTAATAGATTGCTCCTTACGAGGAGACG | 819 |
| | ATTCTCAAACAAATGTGTC | |
| R3052_CasPhi12 | CTTTCAAGACTAATAGATTGCTCCTTACGAGGAGACA | 820 |
| | TTCTCAAACAAATGTGTCA | |
| R3053_CasPhi12 | CTTTCAAGACTAATAGATTGCTCCTTACGAGGAGACT | 821 |
| | GACACATTTGTTTGAGAAT | |
| R3054_CasPhi12 | CTTTCAAGACTAATAGATTGCTCCTTACGAGGAGACTC | 822 |
| | AAACAAATGTGTCACAAA | |
| R3055_CasPhi12 | CTTTCAAGACTAATAGATTGCTCCTTACGAGGAGACG | 823 |
| | TGACACATTTGTTTGAGAA | |
| R3056_CasPhi12 | CTTTCAAGACTAATAGATTGCTCCTTACGAGGAGACCT | 824 |
| | TTGTGACACATTTGTTTG | |
| R3057_CasPhi12 | CTTTCAAGACTAATAGATTGCTCCTTACGAGGAGACT | 825 |
| | GATGTGTATATCACAGACA | |
| R3058_CasPhi12 | CTTTCAAGACTAATAGATTGCTCCTTACGAGGAGACTC | 826 |
| | TGTGATATACACATCAGA | |
| R3059_CasPhi12 | CTTTCAAGACTAATAGATTGCTCCTTACGAGGAGACG | 827 |
| | TCTGTGATATACACATCAG | |
| R3060_CasPhi12 | CTTTCAAGACTAATAGATTGCTCCTTACGAGGAGACT | 828 |
| | GTCTGTGATATACACATCA | |
| R3061_CasPhi12 | CTTTCAAGACTAATAGATTGCTCCTTACGAGGAGACA | 829 |
| | AGTCCATAGACCTCATGTC | |
| R3062_CasPhi12 | CTTTCAAGACTAATAGATTGCTCCTTACGAGGAGACCT | 830 |
| | CTTGAAGTCCATAGACCT | |
| R3063_CasPhi12 | CTTTCAAGACTAATAGATTGCTCCTTACGAGGAGACA | 831 |
| | AGAGCAACAGTGCTGTGGC | |
| R3064_CasPhi12 | CTTTCAAGACTAATAGATTGCTCCTTACGAGGAGACCT | 832 |
| | CCAGGCCACAGCACTGTT | |
| R3065_CasPhi12 | CTTTCAAGACTAATAGATTGCTCCTTACGAGGAGACTT | 833 |
| | GCTCCAGGCCACAGCACT | |
| R3066_CasPhi12 | CTTTCAAGACTAATAGATTGCTCCTTACGAGGAGACG | 834 |
| | TTGCTCCAGGCCACAGCAC | |
| R3067_CasPhi12 | CTTTCAAGACTAATAGATTGCTCCTTACGAGGAGACC | 835 |
| | ACATGCAAAGTCAGATTTG | |
| R3068_CasPhi12 | CTTTCAAGACTAATAGATTGCTCCTTACGAGGAGACG | 836 |
| | CACATGCAAAGTCAGATTT | |
| R3069_CasPhi12 | CTTTCAAGACTAATAGATTGCTCCTTACGAGGAGACG | 837 |
| | CATGTGCAAACGCCTTCAA | |
| R3070_CasPhi12 | CTTTCAAGACTAATAGATTGCTCCTTACGAGGAGACA | 838 |
| | AGGCGTTTGCACATGCAAA | |
| R3071_CasPhi12 | CTTTCAAGACTAATAGATTGCTCCTTACGAGGAGACC | 839 |
| | ATGTGCAAACGCCTTCAAC | |
| R3072_CasPhi12 | CTTTCAAGACTAATAGATTGCTCCTTACGAGGAGACTT | 840 |
| | GAAGGCGTTTGCACATGC | |
| R3073_CasPhi12 | CTTTCAAGACTAATAGATTGCTCCTTACGAGGAGACA | 841 |
| | ACAACAGCATTATTCCAGA | |
| R3074_CasPhi12 | CTTTCAAGACTAATAGATTGCTCCTTACGAGGAGACT | 842 |
| | GGAATAATGCTGTTGTTGA | |
| R3075_CasPhi12 | CTTTCAAGACTAATAGATTGCTCCTTACGAGGAGACTT | 843 |
| | CCAGAAGACACCTTCTTC | |
| R3076_CasPhi12 | CTTTCAAGACTAATAGATTGCTCCTTACGAGGAGACC | 844 |
| | AGAAGACACCTTCTTCCCC | |
| R3077_CasPhi12 | CTTTCAAGACTAATAGATTGCTCCTTACGAGGAGACC | 845 |
| | CTGGGCTGGGGAAGAAGGT | |
| R3078_CasPhi12 | CTTTCAAGACTAATAGATTGCTCCTTACGAGGAGACTT | 846 |
| | CCCCAGCCCAGGTAAGGG | |
| R3079_CasPhi12 | CTTTCAAGACTAATAGATTGCTCCTTACGAGGAGACC | 847 |
| | CCAGCCCAGGTAAGGGCAG | |
| R3080_CasPhi12 | CTTTCAAGACTAATAGATTGCTCCTTACGAGGAGACT | 848 |
| | AAAAGGAAAAACAGACATT | |
| R3081_CasPhi12 | CTTTCAAGACTAATAGATTGCTCCTTACGAGGAGACCT | 849 |
| | AAAAGGAAAAACAGACAT | |
| R3082_CasPhi12 | CTTTCAAGACTAATAGATTGCTCCTTACGAGGAGACTT | 850 |
| | CCTTTTAGAAAGTTCCTG | |
| R3083_CasPhi12 | CTTTCAAGACTAATAGATTGCTCCTTACGAGGAGACTC | 851 |
| | CTTTTAGAAAGTTCCTGT | |
| R3084_CasPhi12 | CTTTCAAGACTAATAGATTGCTCCTTACGAGGAGACC | 852 |
| | CTTTTAGAAAGTTCCTGTG | |
| R3085_CasPhi12 | CTTTCAAGACTAATAGATTGCTCCTTACGAGGAGACCT | 853 |
| | TTTAGAAAGTTCCTGTGA | |
| R3086_CasPhi12 | CTTTCAAGACTAATAGATTGCTCCTTACGAGGAGACT | 854 |
| | AGAAAGTTCCTGTGATGTC | |
| R3136_CasPhi12 | CTTTCAAGACTAATAGATTGCTCCTTACGAGGAGACA | 855 |
| | GAAAGTTCCTGTGATGTCA | |
| R3137_CasPhi12 | CTTTCAAGACTAATAGATTGCTCCTTACGAGGAGACG | 856 |
| | AAAGTTCCTGTGATGTCAA | |
| R3138_CasPhi12 | CTTTCAAGACTAATAGATTGCTCCTTACGAGGAGACA | 857 |
| | CATCACAGGAACTTTCTAA | |
| R3139_CasPhi12 | CTTTCAAGACTAATAGATTGCTCCTTACGAGGAGACCT | 858 |
| | GTGATGTCAAGCTGGTCG | |
| R3140_CasPhi12 | CTTTCAAGACTAATAGATTGCTCCTTACGAGGAGACTC | 859 |
| | GACCAGCTTGACATCACA | |
| R3141_CasPhi12 | CTTTCAAGACTAATAGATTGCTCCTTACGAGGAGACCT | 860 |
| | CGACCAGCTTGACATCAC | |
| R3142_CasPhi12 | CTTTCAAGACTAATAGATTGCTCCTTACGAGGAGACTC | 861 |
| | TCGACCAGCTTGACATCA | |
| R3143_CasPhi12 | CTTTCAAGACTAATAGATTGCTCCTTACGAGGAGACA | 862 |
| | AAGCTTTTCTCGACCAGCT | |
| R3144_CasPhi12 | CTTTCAAGACTAATAGATTGCTCCTTACGAGGAGACC | 863 |
| | AAAGCTTTTCTCGACCAGC | |
| R3145_CasPhi12 | CTTTCAAGACTAATAGATTGCTCCTTACGAGGAGACC | 864 |
| | CTGTTTCAAAGCTTTTCTC | |
| R3146_CasPhi12 | CTTTCAAGACTAATAGATTGCTCCTTACGAGGAGACG | 865 |
| | AAACAGGTAAGACAGGGGT | |
| R3147_CasPhi12 | CTTTCAAGACTAATAGATTGCTCCTTACGAGGAGACA | 866 |
| | AACAGGTAAGACAGGGGTC | |

TABLE 9.1
CasΦ.12 gRNAs targeting human TRAC in T cells

| SEQ ID NO | Repeat + spacer RNA Sequence (5′ --> 3′) |
|---|---|
| 2096 | CUUUCAAGACUAAUAGAUUGCUCCUUACGAGGAGACU |
| | GGAUAUCUGUGGGACAAGA |
| 2097 | CUUUCAAGACUAAUAGAUUGCUCCUUACGAGGAGACU |
| | CCCACAGAUAUCCAGAACC |
| 2098 | CUUUCAAGACUAAUAGAUUGCUCCUUACGAGGAGACG |
| | AGUCUCUCAGCUGGUACAC |
| 2099 | CUUUCAAGACUAAUAGAUUGCUCCUUACGAGGAGACA |
| | GAGUCUCUCAGCUGGUACA |
| 2100 | CUUUCAAGACUAAUAGAUUGCUCCUUACGAGGAGACU |
| | CACUGGAUUUAGAGUCUCU |
| 2101 | CUUUCAAGACUAAUAGAUUGCUCCUUACGAGGAGACA |
| | GAAUCAAAAUCGGUGAAUA |
| 2102 | CUUUCAAGACUAAUAGAUUGCUCCUUACGAGGAGACG |
| | AGAAUCAAAAUCGGUGAAU |
| 2103 | CUUUCAAGACUAAUAGAUUGCUCCUUACGAGGAGACA |
| | CCGAUUUUGAUUCUCAAAC |
| 2104 | CUUUCAAGACUAAUAGAUUGCUCCUUACGAGGAGACU |
| | UUGAGAAUCAAAAUCGGUG |
| 2105 | CUUUCAAGACUAAUAGAUUGCUCCUUACGAGGAGACG |
| | UUUGAGAAUCAAAAUCGGU |
| 2106 | CUUUCAAGACUAAUAGAUUGCUCCUUACGAGGAGACU |
| | GAUUCUCAAACAAAUGUGU |
| 2107 | CUUUCAAGACUAAUAGAUUGCUCCUUACGAGGAGACG |
| | AUUCUCAAACAAAUGUGUC |
| 2108 | CUUUCAAGACUAAUAGAUUGCUCCUUACGAGGAGACA |
| | UUCUCAAACAAAUGUGUCA |
| 2109 | CUUUCAAGACUAAUAGAUUGCUCCUUACGAGGAGACU |
| | GACACAUUUGUUUGAGAAU |
| 2110 | CUUUCAAGACUAAUAGAUUGCUCCUUACGAGGAGACU |
| | CAAACAAAUGUGUCACAAA |
| 2111 | CUUUCAAGACUAAUAGAUUGCUCCUUACGAGGAGACG |
| | UGACACAUUUGUUUGAGAA |
| 2112 | CUUUCAAGACUAAUAGAUUGCUCCUUACGAGGAGACC |
| | UUUGUGACACAUUUGUUUG |
| 2113 | CUUUCAAGACUAAUAGAUUGCUCCUUACGAGGAGACU |
| | GAUGUGUAUAUCACAGACA |
| 2114 | CUUUCAAGACUAAUAGAUUGCUCCUUACGAGGAGACU |
| | CUGUGAUAUACACAUCAGA |
| 2115 | CUUUCAAGACUAAUAGAUUGCUCCUUACGAGGAGACG |
| | UCUGUGAUAUACACAUCAG |
| 2116 | CUUUCAAGACUAAUAGAUUGCUCCUUACGAGGAGACU |
| | GUCUGUGAUAUACACAUCA |
| 2117 | CUUUCAAGACUAAUAGAUUGCUCCUUACGAGGAGACA |
| | AGUCCAUAGACCUCAUGUC |
| 2118 | CUUUCAAGACUAAUAGAUUGCUCCUUACGAGGAGACC |
| | UCUUGAAGUCCAUAGACCU |
| 2119 | CUUUCAAGACUAAUAGAUUGCUCCUUACGAGGAGACA |
| | AGAGCAACAGUGCUGUGGC |
| 2120 | CUUUCAAGACUAAUAGAUUGCUCCUUACGAGGAGACC |
| | UCCAGGCCACAGCACUGUU |
| 2121 | CUUUCAAGACUAAUAGAUUGCUCCUUACGAGGAGACU |
| | UGCUCCAGGCCACAGCACU |
| 2122 | CUUUCAAGACUAAUAGAUUGCUCCUUACGAGGAGACG |
| | UUGCUCCAGGCCACAGCAC |
| 2123 | CUUUCAAGACUAAUAGAUUGCUCCUUACGAGGAGACC |
| | ACAUGCAAAGUCAGAUUUG |
| 2124 | CUUUCAAGACUAAUAGAUUGCUCCUUACGAGGAGACG |
| | CACAUGCAAAGUCAGAUUU |
| 2125 | CUUUCAAGACUAAUAGAUUGCUCCUUACGAGGAGACG |
| | CAUGUGCAAACGCCUUCAA |
| 2126 | CUUUCAAGACUAAUAGAUUGCUCCUUACGAGGAGACA |
| | AGGCGUUUGCACAUGCAAA |
| 2127 | CUUUCAAGACUAAUAGAUUGCUCCUUACGAGGAGACC |
| | AUGUGCAAACGCCUUCAAC |
| 2128 | CUUUCAAGACUAAUAGAUUGCUCCUUACGAGGAGACU |
| | UGAAGGCGUUUGCACAUGC |
| 2129 | CUUUCAAGACUAAUAGAUUGCUCCUUACGAGGAGACA |
| | ACAACAGCAUUAUUCCAGA |
| 2130 | CUUUCAAGACUAAUAGAUUGCUCCUUACGAGGAGACU |
| | GGAAUAAUGCUGUUGUUGA |
| 2131 | CUUUCAAGACUAAUAGAUUGCUCCUUACGAGGAGACU |
| | UCCAGAAGACACCUUCUUC |
| 2132 | CUUUCAAGACUAAUAGAUUGCUCCUUACGAGGAGACC |
| | AGAAGACACCUUCUUCCCC |
| 2133 | CUUUCAAGACUAAUAGAUUGCUCCUUACGAGGAGACC |
| | CUGGGCUGGGGAAGAAGGU |
| 2134 | CUUUCAAGACUAAUAGAUUGCUCCUUACGAGGAGACU |
| | UCCCCAGCCCAGGUAAGGG |
| 2135 | CUUUCAAGACUAAUAGAUUGCUCCUUACGAGGAGACC |
| | CCAGCCCAGGUAAGGGCAG |
| 2136 | CUUUCAAGACUAAUAGAUUGCUCCUUACGAGGAGACU |
| | AAAAGGAAAAACAGACAUU |
| 2137 | CUUUCAAGACUAAUAGAUUGCUCCUUACGAGGAGACC |
| | UAAAAGGAAAAACAGACAU |
| 2138 | CUUUCAAGACUAAUAGAUUGCUCCUUACGAGGAGACU |
| | UCCUUUUAGAAAGUUCCUG |
| 2139 | CUUUCAAGACUAAUAGAUUGCUCCUUACGAGGAGACU |
| | CCUUUUAGAAAGUUCCUGU |
| 2140 | CUUUCAAGACUAAUAGAUUGCUCCUUACGAGGAGACC |
| | CUUUUAGAAAGUUCCUGUG |
| 2141 | CUUUCAAGACUAAUAGAUUGCUCCUUACGAGGAGACC |
| | UUUUAGAAAGUUCCUGUGA |
| 2142 | CUUUCAAGACUAAUAGAUUGCUCCUUACGAGGAGACU |
| | AGAAAGUUCCUGUGAUGUC |
| 2143 | CUUUCAAGACUAAUAGAUUGCUCCUUACGAGGAGACA |
| | GAAAGUUCCUGUGAUGUCA |
| 2144 | CUUUCAAGACUAAUAGAUUGCUCCUUACGAGGAGACG |
| | AAAGUUCCUGUGAUGUCAA |
| 2145 | CUUUCAAGACUAAUAGAUUGCUCCUUACGAGGAGACA |
| | CAUCACAGGAACUUUCUAA |
| 2146 | CUUUCAAGACUAAUAGAUUGCUCCUUACGAGGAGACC |
| | UGUGAUGUCAAGCUGGUCG |
| 2147 | CUUUCAAGACUAAUAGAUUGCUCCUUACGAGGAGACU |
| | CGACCAGCUUGACAUCACA |
| 2148 | CUUUCAAGACUAAUAGAUUGCUCCUUACGAGGAGACC |
| | UCGACCAGCUUGACAUCAC |
| 2149 | CUUUCAAGACUAAUAGAUUGCUCCUUACGAGGAGACU |
| | CUCGACCAGCUUGACAUCA |
| 2150 | CUUUCAAGACUAAUAGAUUGCUCCUUACGAGGAGACA |
| | AAGCUUUUCUCGACCAGCU |
| 2151 | CUUUCAAGACUAAUAGAUUGCUCCUUACGAGGAGACC |
| | AAAGCUUUUCUCGACCAGC |
| 2152 | CUUUCAAGACUAAUAGAUUGCUCCUUACGAGGAGACC |
| | CUGUUUCAAAGCUUUUCUC |
| 2153 | CUUUCAAGACUAAUAGAUUGCUCCUUACGAGGAGACG |
| | AAACAGGUAAGACAGGGGU |
| 2154 | CUUUCAAGACUAAUAGAUUGCUCCUUACGAGGAGACA |
| | AACAGGUAAGACAGGGGUC |

TABLE 10
CasΦ.32 gRNAs (DNA sequences) targeting human TRAC in T cells

| | Repeat + spacer RNA Sequence (5′ --> 3′), | SEQ ID |
|---|---|---|
| Name | shown as DNA | NO |
| R3040_CasPhi32 | GCTGGGGACCGATCCTGATTGCTCGCTGCGGCG | 867 |
| | AGACTGGATATCTGTGGGACAAGA | |
| R3041_CasPhi32 | GCTGGGGACCGATCCTGATTGCTCGCTGCGGCG | 868 |
| | AGACTCCCACAGATATCCAGAACC | |
| R3042_CasPhi32 | GCTGGGGACCGATCCTGATTGCTCGCTGCGGCG | 869 |
| | AGACGAGTCTCTCAGCTGGTACAC | |
| R3043_CasPhi32 | GCTGGGGACCGATCCTGATTGCTCGCTGCGGCG | 870 |
| | AGACAGAGTCTCTCAGCTGGTACA | |
| R3044_CasPhi32 | GCTGGGGACCGATCCTGATTGCTCGCTGCGGCG | 871 |
| | AGACTCACTGGATTTAGAGTCTCT | |
| R3045_CasPhi32 | GCTGGGGACCGATCCTGATTGCTCGCTGCGGCG | 872 |
| | AGACAGAATCAAAATCGGTGAATA | |
| R3046_CasPhi32 | GCTGGGGACCGATCCTGATTGCTCGCTGCGGCG | 873 |
| | AGACGAGAATCAAAATCGGTGAAT | |
| R3047_CasPhi32 | GCTGGGGACCGATCCTGATTGCTCGCTGCGGCG | 874 |
| | AGACACCGATTTTGATTCTCAAAC | |
| R3048_CasPhi32 | GCTGGGGACCGATCCTGATTGCTCGCTGCGGCG | 875 |
| | AGACTTTGAGAATCAAAATCGGTG | |
| R3049_CasPhi32 | GCTGGGGACCGATCCTGATTGCTCGCTGCGGCG | 876 |
| | AGACGTTTGAGAATCAAAATCGGT | |
| R3050_CasPhi32 | GCTGGGGACCGATCCTGATTGCTCGCTGCGGCG | 877 |
| | AGACTGATTCTCAAACAAATGTGT | |
| R3051_CasPhi32 | GCTGGGGACCGATCCTGATTGCTCGCTGCGGCG | 878 |
| | AGACGATTCTCAAACAAATGTGTC | |
| R3052_CasPhi32 | GCTGGGGACCGATCCTGATTGCTCGCTGCGGCG | 879 |
| | AGACATTCTCAAACAAATGTGTCA | |
| R3053_CasPhi32 | GCTGGGGACCGATCCTGATTGCTCGCTGCGGCG | 880 |
| | AGACTGACACATTTGTTTGAGAAT | |
| R3054_CasPhi32 | GCTGGGGACCGATCCTGATTGCTCGCTGCGGCG | 881 |
| | AGACTCAAACAAATGTGTCACAAA | |
| R3055_CasPhi32 | GCTGGGGACCGATCCTGATTGCTCGCTGCGGCG | 882 |
| | AGACGTGACACATTTGTTTGAGAA | |
| R3056_CasPhi32 | GCTGGGGACCGATCCTGATTGCTCGCTGCGGCG | 883 |
| | AGACCTTTGTGACACATTTGTTTG | |
| R3057_CasPhi32 | GCTGGGGACCGATCCTGATTGCTCGCTGCGGCG | 884 |
| | AGACTGATGTGTATATCACAGACA | |
| R3058_CasPhi32 | GCTGGGGACCGATCCTGATTGCTCGCTGCGGCG | 885 |
| | AGACTCTGTGATATACACATCAGA | |
| R3059_CasPhi32 | GCTGGGGACCGATCCTGATTGCTCGCTGCGGCG | 886 |
| | AGACGTCTGTGATATACACATCAG | |
| R3060_CasPhi32 | GCTGGGGACCGATCCTGATTGCTCGCTGCGGCG | 887 |
| | AGACTGTCTGTGATATACACATCA | |
| R3061_CasPhi32 | GCTGGGGACCGATCCTGATTGCTCGCTGCGGCG | 888 |
| | AGACAAGTCCATAGACCTCATGTC | |
| R3062_CasPhi32 | GCTGGGGACCGATCCTGATTGCTCGCTGCGGCG | 889 |
| | AGACCTCTTGAAGTCCATAGACCT | |
| R3063_CasPhi32 | GCTGGGGACCGATCCTGATTGCTCGCTGCGGCG | 890 |
| | AGACAAGAGCAACAGTGCTGTGGC | |
| R3064_CasPhi32 | GCTGGGGACCGATCCTGATTGCTCGCTGCGGCG | 891 |
| | AGACCTCCAGGCCACAGCACTGTT | |
| R3065_CasPhi32 | GCTGGGGACCGATCCTGATTGCTCGCTGCGGCG | 892 |
| | AGACTTGCTCCAGGCCACAGCACT | |
| R3066_CasPhi32 | GCTGGGGACCGATCCTGATTGCTCGCTGCGGCG | 893 |
| | AGACGTTGCTCCAGGCCACAGCAC | |
| R3067_CasPhi32 | GCTGGGGACCGATCCTGATTGCTCGCTGCGGCG | 894 |
| | AGACCACATGCAAAGTCAGATTTG | |
| R3068_CasPhi32 | GCTGGGGACCGATCCTGATTGCTCGCTGCGGCG | 895 |
| | AGACGCACATGCAAAGTCAGATTT | |
| R3069_CasPhi32 | GCTGGGGACCGATCCTGATTGCTCGCTGCGGCG | 896 |
| | AGACGCATGTGCAAACGCCTTCAA | |
| R3070_CasPhi32 | GCTGGGGACCGATCCTGATTGCTCGCTGCGGCG | 897 |
| | AGACAAGGCGTTTGCACATGCAAA | |
| R3071_CasPhi32 | GCTGGGGACCGATCCTGATTGCTCGCTGCGGCG | 898 |
| | AGACCATGTGCAAACGCCTTCAAC | |
| R3072_CasPhi32 | GCTGGGGACCGATCCTGATTGCTCGCTGCGGCG | 899 |
| | AGACTTGAAGGCGTTTGCACATGC | |
| R3073_CasPhi32 | GCTGGGGACCGATCCTGATTGCTCGCTGCGGCG | 900 |
| | AGACAACAACAGCATTATTCCAGA | |
| R3074_CasPhi32 | GCTGGGGACCGATCCTGATTGCTCGCTGCGGCG | 901 |
| | AGACTGGAATAATGCTGTTGTTGA | |
| R3075_CasPhi32 | GCTGGGGACCGATCCTGATTGCTCGCTGCGGCG | 902 |
| | AGACTTCCAGAAGACACCTTCTTC | |
| R3076_CasPhi32 | GCTGGGGACCGATCCTGATTGCTCGCTGCGGCG | 903 |
| | AGACCAGAAGACACCTTCTTCCCC | |
| R3077_CasPhi32 | GCTGGGGACCGATCCTGATTGCTCGCTGCGGCG | 904 |
| | AGACCCTGGGCTGGGGAAGAAGGT | |
| R3078_CasPhi32 | GCTGGGGACCGATCCTGATTGCTCGCTGCGGCG | 905 |
| | AGACTTCCCCAGCCCAGGTAAGGG | |
| R3079_CasPhi32 | GCTGGGGACCGATCCTGATTGCTCGCTGCGGCG | 906 |
| | AGACCCCAGCCCAGGTAAGGGCAG | |
| R3080_CasPhi32 | GCTGGGGACCGATCCTGATTGCTCGCTGCGGCG | 907 |
| | AGACTAAAAGGAAAAACAGACATT | |
| R3081_CasPhi32 | GCTGGGGACCGATCCTGATTGCTCGCTGCGGCG | 908 |
| | AGACCTAAAAGGAAAAACAGACAT | |
| R3082_CasPhi32 | GCTGGGGACCGATCCTGATTGCTCGCTGCGGCG | 909 |
| | AGACTTCCTTTTAGAAAGTTCCTG | |
| R3083_CasPhi32 | GCTGGGGACCGATCCTGATTGCTCGCTGCGGCG | 910 |
| | AGACTCCTTTTAGAAAGTTCCTGT | |
| R3084_CasPhi32 | GCTGGGGACCGATCCTGATTGCTCGCTGCGGCG | 911 |
| | AGACCCTTTTAGAAAGTTCCTGTG | |
| R3085_CasPhi32 | GCTGGGGACCGATCCTGATTGCTCGCTGCGGCG | 912 |
| | AGACCTTTTAGAAAGTTCCTGTGA | |
| R3086_CasPhi32 | GCTGGGGACCGATCCTGATTGCTCGCTGCGGCG | 913 |
| | AGACTAGAAAGTTCCTGTGATGTC | |
| R3136_CasPhi32 | GCTGGGGACCGATCCTGATTGCTCGCTGCGGCG | 914 |
| | AGACAGAAAGTTCCTGTGATGTCA | |
| R3137_CasPhi32 | GCTGGGGACCGATCCTGATTGCTCGCTGCGGCG | 915 |
| | AGACGAAAGTTCCTGTGATGTCAA | |
| R3138_CasPhi32 | GCTGGGGACCGATCCTGATTGCTCGCTGCGGCG | 916 |
| | AGACACATCACAGGAACTTTCTAA | |
| R3139_CasPhi32 | GCTGGGGACCGATCCTGATTGCTCGCTGCGGCG | 917 |
| | AGACCTGTGATGTCAAGCTGGTCG | |
| R3140_CasPhi32 | GCTGGGGACCGATCCTGATTGCTCGCTGCGGCG | 918 |
| | AGACTCGACCAGCTTGACATCACA | |
| R3141_CasPhi32 | GCTGGGGACCGATCCTGATTGCTCGCTGCGGCG | 919 |
| | AGACCTCGACCAGCTTGACATCAC | |
| R3142_CasPhi32 | GCTGGGGACCGATCCTGATTGCTCGCTGCGGCG | 920 |
| | AGACTCTCGACCAGCTTGACATCA | |
| R3143_CasPhi32 | GCTGGGGACCGATCCTGATTGCTCGCTGCGGCG | 921 |
| | AGACAAAGCTTTTCTCGACCAGCT | |
| R3144_CasPhi32 | GCTGGGGACCGATCCTGATTGCTCGCTGCGGCG | 922 |
| | AGACCAAAGCTTTTCTCGACCAGC | |
| R3145_CasPhi32 | GCTGGGGACCGATCCTGATTGCTCGCTGCGGCG | 923 |
| | AGACCCTGTTTCAAAGCTTTTCTC | |
| R3146_CasPhi32 | GCTGGGGACCGATCCTGATTGCTCGCTGCGGCG | 924 |
| | AGACGAAACAGGTAAGACAGGGGT | |
| R3147_CasPhi32 | GCTGGGGACCGATCCTGATTGCTCGCTGCGGCG | 925 |
| | AGACAAACAGGTAAGACAGGGGTC | |

TABLE 10.1
CasΦ.32 gRNAs targeting human TRAC in T cells

| SEQ ID NO | Repeat + spacer RNA Sequence (5′ --> 3′) |
|---|---|
| 2155 | GCUGGGGACCGAUCCUGAUUGCUCGCUGCGGCGAGACU |
| | GGAUAUCUGUGGGACAAGA |
| 2156 | GCUGGGGACCGAUCCUGAUUGCUCGCUGCGGCGAGACU |
| | CCCACAGAUAUCCAGAACC |
| 2157 | GCUGGGGACCGAUCCUGAUUGCUCGCUGCGGCGAGACG |
| | AGUCUCUCAGCUGGUACAC |
| 2158 | GCUGGGGACCGAUCCUGAUUGCUCGCUGCGGCGAGACA |
| | GAGUCUCUCAGCUGGUACA |
| 2159 | GCUGGGGACCGAUCCUGAUUGCUCGCUGCGGCGAGACU |
| | CACUGGAUUUAGAGUCUCU |
| 2160 | GCUGGGGACCGAUCCUGAUUGCUCGCUGCGGCGAGACA |
| | GAAUCAAAAUCGGUGAAUA |
| 2161 | GCUGGGGACCGAUCCUGAUUGCUCGCUGCGGCGAGACG |
| | AGAAUCAAAAUCGGUGAAU |
| 2162 | GCUGGGGACCGAUCCUGAUUGCUCGCUGCGGCGAGACA |
| | CCGAUUUUGAUUCUCAAAC |
| 2163 | GCUGGGGACCGAUCCUGAUUGCUCGCUGCGGCGAGACU |
| | UUGAGAAUCAAAAUCGGUG |
| 2164 | GCUGGGGACCGAUCCUGAUUGCUCGCUGCGGCGAGACG |
| | UUUGAGAAUCAAAAUCGGU |
| 2165 | GCUGGGGACCGAUCCUGAUUGCUCGCUGCGGCGAGACU |
| | GAUUCUCAAACAAAUGUGU |
| 2166 | GCUGGGGACCGAUCCUGAUUGCUCGCUGCGGCGAGACG |
| | AUUCUCAAACAAAUGUGUC |
| 2167 | GCUGGGGACCGAUCCUGAUUGCUCGCUGCGGCGAGACA |
| | UUCUCAAACAAAUGUGUCA |
| 2168 | GCUGGGGACCGAUCCUGAUUGCUCGCUGCGGCGAGACU |
| | GACACAUUUGUUUGAGAAU |
| 2169 | GCUGGGGACCGAUCCUGAUUGCUCGCUGCGGCGAGACU |
| | CAAACAAAUGUGUCACAAA |
| 2170 | GCUGGGGACCGAUCCUGAUUGCUCGCUGCGGCGAGACG |
| | UGACACAUUUGUUUGAGAA |
| 2171 | GCUGGGGACCGAUCCUGAUUGCUCGCUGCGGCGAGACC |
| | UUUGUGACACAUUUGUUUG |
| 2172 | GCUGGGGACCGAUCCUGAUUGCUCGCUGCGGCGAGACU |
| | GAUGUGUAUAUCACAGACA |
| 2173 | GCUGGGGACCGAUCCUGAUUGCUCGCUGCGGCGAGACU |
| | CUGUGAUAUACACAUCAGA |
| 2174 | GCUGGGGACCGAUCCUGAUUGCUCGCUGCGGCGAGACG |
| | UCUGUGAUAUACACAUCAG |
| 2175 | GCUGGGGACCGAUCCUGAUUGCUCGCUGCGGCGAGACU |
| | GUCUGUGAUAUACACAUCA |
| 2176 | GCUGGGGACCGAUCCUGAUUGCUCGCUGCGGCGAGACA |
| | AGUCCAUAGACCUCAUGUC |
| 2177 | GCUGGGGACCGAUCCUGAUUGCUCGCUGCGGCGAGACC |
| | UCUUGAAGUCCAUAGACCU |
| 2178 | GCUGGGGACCGAUCCUGAUUGCUCGCUGCGGCGAGACA |
| | AGAGCAACAGUGCUGUGGC |
| 2179 | GCUGGGGACCGAUCCUGAUUGCUCGCUGCGGCGAGACC |
| | UCCAGGCCACAGCACUGUU |
| 2180 | GCUGGGGACCGAUCCUGAUUGCUCGCUGCGGCGAGACU |
| | UGCUCCAGGCCACAGCACU |
| 2181 | GCUGGGGACCGAUCCUGAUUGCUCGCUGCGGCGAGACG |
| | UUGCUCCAGGCCACAGCAC |
| 2182 | GCUGGGGACCGAUCCUGAUUGCUCGCUGCGGCGAGACC |
| | ACAUGCAAAGUCAGAUUUG |
| 2183 | GCUGGGGACCGAUCCUGAUUGCUCGCUGCGGCGAGACG |
| | CACAUGCAAAGUCAGAUUU |
| 2184 | GCUGGGGACCGAUCCUGAUUGCUCGCUGCGGCGAGACG |
| | CAUGUGCAAACGCCUUCAA |
| 2185 | GCUGGGGACCGAUCCUGAUUGCUCGCUGCGGCGAGACA |
| | AGGCGUUUGCACAUGCAAA |
| 2186 | GCUGGGGACCGAUCCUGAUUGCUCGCUGCGGCGAGACC |
| | AUGUGCAAACGCCUUCAAC |
| 2187 | GCUGGGGACCGAUCCUGAUUGCUCGCUGCGGCGAGACU |
| | UGAAGGCGUUUGCACAUGC |
| 2188 | GCUGGGGACCGAUCCUGAUUGCUCGCUGCGGCGAGACA |
| | ACAACAGCAUUAUUCCAGA |
| 2189 | GCUGGGGACCGAUCCUGAUUGCUCGCUGCGGCGAGACU |
| | GGAAUAAUGCUGUUGUUGA |
| 2190 | GCUGGGGACCGAUCCUGAUUGCUCGCUGCGGCGAGACU |
| | UCCAGAAGACACCUUCUUC |
| 2191 | GCUGGGGACCGAUCCUGAUUGCUCGCUGCGGCGAGACC |
| | AGAAGACACCUUCUUCCCC |
| 2192 | GCUGGGGACCGAUCCUGAUUGCUCGCUGCGGCGAGACC |
| | CUGGGCUGGGGAAGAAGGU |
| 2193 | GCUGGGGACCGAUCCUGAUUGCUCGCUGCGGCGAGACU |
| | UCCCCAGCCCAGGUAAGGG |
| 2194 | GCUGGGGACCGAUCCUGAUUGCUCGCUGCGGCGAGACC |
| | CCAGCCCAGGUAAGGGCAG |
| 2195 | GCUGGGGACCGAUCCUGAUUGCUCGCUGCGGCGAGACU |
| | AAAAGGAAAAACAGACAUU |
| 2196 | GCUGGGGACCGAUCCUGAUUGCUCGCUGCGGCGAGACC |
| | UAAAAGGAAAAACAGACAU |
| 2197 | GCUGGGGACCGAUCCUGAUUGCUCGCUGCGGCGAGACU |
| | UCCUUUUAGAAAGUUCCUG |
| 2198 | GCUGGGGACCGAUCCUGAUUGCUCGCUGCGGCGAGACU |
| | CCUUUUAGAAAGUUCCUGU |
| 2199 | GCUGGGGACCGAUCCUGAUUGCUCGCUGCGGCGAGACC |
| | CUUUUAGAAAGUUCCUGUG |
| 2200 | GCUGGGGACCGAUCCUGAUUGCUCGCUGCGGCGAGACC |
| | UUUUAGAAAGUUCCUGUGA |
| 2201 | GCUGGGGACCGAUCCUGAUUGCUCGCUGCGGCGAGACU |
| | AGAAAGUUCCUGUGAUGUC |
| 2202 | GCUGGGGACCGAUCCUGAUUGCUCGCUGCGGCGAGACA |
| | GAAAGUUCCUGUGAUGUCA |
| 2203 | GCUGGGGACCGAUCCUGAUUGCUCGCUGCGGCGAGACG |
| | AAAGUUCCUGUGAUGUCAA |
| 2204 | GCUGGGGACCGAUCCUGAUUGCUCGCUGCGGCGAGACA |
| | CAUCACAGGAACUUUCUAA |
| 2205 | GCUGGGGACCGAUCCUGAUUGCUCGCUGCGGCGAGACC |
| | UGUGAUGUCAAGCUGGUCG |
| 2206 | GCUGGGGACCGAUCCUGAUUGCUCGCUGCGGCGAGACU |
| | CGACCAGCUUGACAUCACA |
| 2207 | GCUGGGGACCGAUCCUGAUUGCUCGCUGCGGCGAGACC |
| | UCGACCAGCUUGACAUCAC |
| 2208 | GCUGGGGACCGAUCCUGAUUGCUCGCUGCGGCGAGACU |
| | CUCGACCAGCUUGACAUCA |
| 2209 | GCUGGGGACCGAUCCUGAUUGCUCGCUGCGGCGAGACA |
| | AAGCUUUUCUCGACCAGCU |
| 2210 | GCUGGGGACCGAUCCUGAUUGCUCGCUGCGGCGAGACC |
| | AAAGCUUUUCUCGACCAGC |
| 2211 | GCUGGGGACCGAUCCUGAUUGCUCGCUGCGGCGAGACC |
| | CUGUUUCAAAGCUUUUCUC |
| 2212 | GCUGGGGACCGAUCCUGAUUGCUCGCUGCGGCGAGACG |
| | AAACAGGUAAGACAGGGGU |
| 2213 | GCUGGGGACCGAUCCUGAUUGCUCGCUGCGGCGAGACA |
| | AACAGGUAAGACAGGGGUC |

TABLE 11
CasΦ.12 gRNAs (DNA sequences) targeting human B2M in T cells

| | Repeat + spacer RNA Sequence (5′ --> 3′), | SEQ ID |
|---|---|---|
| Name | shown as DNA | NO |
| R3087_CasPhi12 | CTTTCAAGACTAATAGATTGCTCCTTACGAGGAG | 926 |
| | ACAATATAAGTGGAGGCGTCGC | |
| R3088_CasPhi12 | CTTTCAAGACTAATAGATTGCTCCTTACGAGGAG | 927 |
| | ACATATAAGTGGAGGCGTCGCG | |
| R3089_CasPhi12 | CTTTCAAGACTAATAGATTGCTCCTTACGAGGAG | 928 |
| | ACAGGAATGCCCGCCAGCGCGA | |
| R3090_CasPhi12 | CTTTCAAGACTAATAGATTGCTCCTTACGAGGAG | 929 |
| | ACCTGAAGCTGACAGCATTCGG | |
| R3091_CasPhi12 | CTTTCAAGACTAATAGATTGCTCCTTACGAGGAG | 930 |
| | ACGGGCCGAGATGTCTCGCTCC | |
| R3092_CasPhi12 | CTTTCAAGACTAATAGATTGCTCCTTACGAGGAG | 931 |
| | ACGCTGTGCTCGCGCTACTCTC | |
| R3093_CasPhi12 | CTTTCAAGACTAATAGATTGCTCCTTACGAGGAG | 932 |
| | ACCTGGCCTGGAGGCTATCCAG | |
| R3094_CasPhi12 | CTTTCAAGACTAATAGATTGCTCCTTACGAGGAG | 933 |
| | ACTGGCCTGGAGGCTATCCAGC | |
| R3095_CasPhi12 | CTTTCAAGACTAATAGATTGCTCCTTACGAGGAG | 934 |
| | ACATGTGTCTTTTCCCGATATT | |
| R3096_CasPhi12 | CTTTCAAGACTAATAGATTGCTCCTTACGAGGAG | 935 |
| | ACTCCCGATATTCCTCAGGTAC | |
| R3097_CasPhi12 | CTTTCAAGACTAATAGATTGCTCCTTACGAGGAG | 936 |
| | ACCCCGATATTCCTCAGGTACT | |
| R3098_CasPhi12 | CTTTCAAGACTAATAGATTGCTCCTTACGAGGAG | 937 |
| | ACCCGATATTCCTCAGGTACTC | |
| R3099_CasPhi12 | CTTTCAAGACTAATAGATTGCTCCTTACGAGGAG | 938 |
| | ACGAGTACCTGAGGAATATCGG | |
| R3100_CasPhi12 | CTTTCAAGACTAATAGATTGCTCCTTACGAGGAG | 939 |
| | ACGGAGTACCTGAGGAATATCG | |
| R3101_CasPhi12 | CTTTCAAGACTAATAGATTGCTCCTTACGAGGAG | 940 |
| | ACCTCAGGTACTCCAAAGATTC | |
| R3102_CasPhi12 | CTTTCAAGACTAATAGATTGCTCCTTACGAGGAG | 941 |
| | ACAGGTTTACTCACGTCATCCA | |
| R3103_CasPhi12 | CTTTCAAGACTAATAGATTGCTCCTTACGAGGAG | 942 |
| | ACACTCACGTCATCCAGCAGAG | |
| R3104_CasPhi12 | CTTTCAAGACTAATAGATTGCTCCTTACGAGGAG | 943 |
| | ACCTCACGTCATCCAGCAGAGA | |
| R3105_CasPhi12 | CTTTCAAGACTAATAGATTGCTCCTTACGAGGAG | 944 |
| | ACTCTGCTGGATGACGTGAGTA | |
| R3106_CasPhi12 | CTTTCAAGACTAATAGATTGCTCCTTACGAGGAG | 945 |
| | ACCATTCTCTGCTGGATGACGT | |
| R3107_CasPhi12 | CTTTCAAGACTAATAGATTGCTCCTTACGAGGAG | 946 |
| | ACCCATTCTCTGCTGGATGACG | |
| R3108_CasPhi12 | CTTTCAAGACTAATAGATTGCTCCTTACGAGGAG | 947 |
| | ACACTTTCCATTCTCTGCTGGA | |
| R3109_CasPhi12 | CTTTCAAGACTAATAGATTGCTCCTTACGAGGAG | 948 |
| | ACGACTTTCCATTCTCTGCTGG | |
| R3110_CasPhi12 | CTTTCAAGACTAATAGATTGCTCCTTACGAGGAG | 949 |
| | ACAGGAAATTTGACTTTCCATT | |
| R3111_CasPhi12 | CTTTCAAGACTAATAGATTGCTCCTTACGAGGAG | 950 |
| | ACCCTGAATTGCTATGTGTCTG | |
| R3112_CasPhi12 | CTTTCAAGACTAATAGATTGCTCCTTACGAGGAG | 951 |
| | ACCTGAATTGCTATGTGTCTGG | |
| R3113_CasPhi12 | CTTTCAAGACTAATAGATTGCTCCTTACGAGGAG | 952 |
| | ACCTATGTGTCTGGGTTTCATC | |
| R3114_CasPhi12 | CTTTCAAGACTAATAGATTGCTCCTTACGAGGAG | 953 |
| | ACAATGTCGGATGGATGAAACC | |
| R3115_CasPhi12 | CTTTCAAGACTAATAGATTGCTCCTTACGAGGAG | 954 |
| | ACCATCCATCCGACATTGAAGT | |
| R3116_CasPhi12 | CTTTCAAGACTAATAGATTGCTCCTTACGAGGAG | 955 |
| | ACATCCATCCGACATTGAAGTT | |
| R3117_CasPhi12 | CTTTCAAGACTAATAGATTGCTCCTTACGAGGAG | 956 |
| | ACAGTAAGTCAACTTCAATGTC | |
| R3118_CasPhi12 | CTTTCAAGACTAATAGATTGCTCCTTACGAGGAG | 957 |
| | ACTTCAGTAAGTCAACTTCAAT | |
| R3119_CasPhi12 | CTTTCAAGACTAATAGATTGCTCCTTACGAGGAG | 958 |
| | ACAAGTTGACTTACTGAAGAAT | |
| R3120_CasPhi12 | CTTTCAAGACTAATAGATTGCTCCTTACGAGGAG | 959 |
| | ACACTTACTGAAGAATGGAGAG | |
| R3121_CasPhi12 | CTTTCAAGACTAATAGATTGCTCCTTACGAGGAG | 960 |
| | ACTCTCTCCATTCTTCAGTAAG | |
| R3122_CasPhi12 | CTTTCAAGACTAATAGATTGCTCCTTACGAGGAG | 961 |
| | ACCTGAAGAATGGAGAGAGAAT | |
| R3123_CasPhi12 | CTTTCAAGACTAATAGATTGCTCCTTACGAGGAG | 962 |
| | ACAATTCTCTCTCCATTCTTCA | |
| R3124_CasPhi12 | CTTTCAAGACTAATAGATTGCTCCTTACGAGGAG | 963 |
| | ACCAATTCTCTCTCCATTCTTC | |
| R3125_CasPhi12 | CTTTCAAGACTAATAGATTGCTCCTTACGAGGAG | 964 |
| | ACTCAATTCTCTCTCCATTCTT | |
| R3126_CasPhi12 | CTTTCAAGACTAATAGATTGCTCCTTACGAGGAG | 965 |
| | ACTTCAATTCTCTCTCCATTCT | |
| R3127_CasPhi12 | CTTTCAAGACTAATAGATTGCTCCTTACGAGGAG | 966 |
| | ACAAAAAGTGGAGCATTCAGAC | |
| R3128_CasPhi12 | CTTTCAAGACTAATAGATTGCTCCTTACGAGGAG | 967 |
| | ACCTGAAAGACAAGTCTGAATG | |
| R3129_CasPhi12 | CTTTCAAGACTAATAGATTGCTCCTTACGAGGAG | 968 |
| | ACAGACTTGTCTTTCAGCAAGG | |
| R3130_CasPhi12 | CTTTCAAGACTAATAGATTGCTCCTTACGAGGAG | 969 |
| | ACTCTTTCAGCAAGGACTGGTC | |
| R3131_CasPhi12 | CTTTCAAGACTAATAGATTGCTCCTTACGAGGAG | 970 |
| | ACCAGCAAGGACTGGTCTTTCT | |
| R3132_CasPhi12 | CTTTCAAGACTAATAGATTGCTCCTTACGAGGAG | 971 |
| | ACAGCAAGGACTGGTCTTTCTA | |
| R3133_CasPhi12 | CTTTCAAGACTAATAGATTGCTCCTTACGAGGAG | 972 |
| | ACCTATCTCTTGTACTACACTG | |
| R3134_CasPhi12 | CTTTCAAGACTAATAGATTGCTCCTTACGAGGAG | 973 |
| | ACTATCTCTTGTACTACACTGA | |
| R3135_CasPhi12 | CTTTCAAGACTAATAGATTGCTCCTTACGAGGAG | 974 |
| | ACAGTGTAGTACAAGAGATAGA | |
| R3148_CasPhi12 | CTTTCAAGACTAATAGATTGCTCCTTACGAGGAG | 975 |
| | ACTACTACACTGAATTCACCCC | |
| R3149_CasPhi12 | CTTTCAAGACTAATAGATTGCTCCTTACGAGGAG | 976 |
| | ACAGTGGGGGTGAATTCAGTGT | |
| R3150_CasPhi12 | CTTTCAAGACTAATAGATTGCTCCTTACGAGGAG | 977 |
| | ACCAGTGGGGGTGAATTCAGTG | |
| R3151_CasPhi12 | CTTTCAAGACTAATAGATTGCTCCTTACGAGGAG | 978 |
| | ACTCAGTGGGGGTGAATTCAGT | |
| R3152_CasPhi12 | CTTTCAAGACTAATAGATTGCTCCTTACGAGGAG | 979 |
| | ACTTCAGTGGGGGTGAATTCAG | |
| R3153_CasPhi12 | CTTTCAAGACTAATAGATTGCTCCTTACGAGGAG | 980 |
| | ACACCCCCACTGAAAAAGATGA | |
| R3154_CasPhi12 | CTTTCAAGACTAATAGATTGCTCCTTACGAGGAG | 981 |
| | ACACACGGCAGGCATACTCATC | |
| R3155_CasPhi12 | CTTTCAAGACTAATAGATTGCTCCTTACGAGGAG | 982 |
| | ACGGCTGTGACAAAGTCACATG | |
| R3156_CasPhi12 | CTTTCAAGACTAATAGATTGCTCCTTACGAGGAG | 983 |
| | ACGTCACAGCCCAAGATAGTTA | |
| R3157_CasPhi12 | CTTTCAAGACTAATAGATTGCTCCTTACGAGGAG | 984 |
| | ACTCACAGCCCAAGATAGTTAA | |
| R3158_CasPhi12 | CTTTCAAGACTAATAGATTGCTCCTTACGAGGAG | 985 |
| | ACACTATCTTGGGCTGTGACAA | |
| R3159_CasPhi12 | CTTTCAAGACTAATAGATTGCTCCTTACGAGGAG | 986 |
| | ACCCCCACTTAACTATCTTGGG | |

TABLE 11.1
CasΦ.12 gRNAs targeting human B2M in T cells

| SEQ ID NO | Repeat + spacer RNA Sequence (5′ --> 3′) |
|---|---|
| 2214 | CUUUCAAGACUAAUAGAUUGCUCCUUACGAGGAGAC |
| | AAUAUAAGUGGAGGCGUCGC |
| 2215 | CUUUCAAGACUAAUAGAUUGCUCCUUACGAGGAGAC |
| | AUAUAAGUGGAGGCGUCGCG |
| 2216 | CUUUCAAGACUAAUAGAUUGCUCCUUACGAGGAGAC |
| | AGGAAUGCCCGCCAGCGCGA |
| 2217 | CUUUCAAGACUAAUAGAUUGCUCCUUACGAGGAGAC |
| | CUGAAGCUGACAGCAUUCGG |
| 2218 | CUUUCAAGACUAAUAGAUUGCUCCUUACGAGGAGAC |
| | GGGCCGAGAUGUCUCGCUCC |
| 2219 | CUUUCAAGACUAAUAGAUUGCUCCUUACGAGGAGAC |
| | GCUGUGCUCGCGCUACUCUC |
| 2220 | CUUUCAAGACUAAUAGAUUGCUCCUUACGAGGAGAC |
| | CUGGCCUGGAGGCUAUCCAG |
| 2221 | CUUUCAAGACUAAUAGAUUGCUCCUUACGAGGAGAC |
| | UGGCCUGGAGGCUAUCCAGC |
| 2222 | CUUUCAAGACUAAUAGAUUGCUCCUUACGAGGAGAC |
| | AUGUGUCUUUUCCCGAUAUU |
| 2223 | CUUUCAAGACUAAUAGAUUGCUCCUUACGAGGAGAC |
| | UCCCGAUAUUCCUCAGGUAC |
| 2224 | CUUUCAAGACUAAUAGAUUGCUCCUUACGAGGAGAC |
| | CCCGAUAUUCCUCAGGUACU |
| 2225 | CUUUCAAGACUAAUAGAUUGCUCCUUACGAGGAGAC |
| | CCGAUAUUCCUCAGGUACUC |
| 2226 | CUUUCAAGACUAAUAGAUUGCUCCUUACGAGGAGAC |
| | GAGUACCUGAGGAAUAUCGG |
| 2227 | CUUUCAAGACUAAUAGAUUGCUCCUUACGAGGAGAC |
| | GGAGUACCUGAGGAAUAUCG |
| 2228 | CUUUCAAGACUAAUAGAUUGCUCCUUACGAGGAGAC |
| | CUCAGGUACUCCAAAGAUUC |
| 2229 | CUUUCAAGACUAAUAGAUUGCUCCUUACGAGGAGAC |
| | AGGUUUACUCACGUCAUCCA |
| 2230 | CUUUCAAGACUAAUAGAUUGCUCCUUACGAGGAGAC |
| | ACUCACGUCAUCCAGCAGAG |
| 2231 | CUUUCAAGACUAAUAGAUUGCUCCUUACGAGGAGAC |
| | CUCACGUCAUCCAGCAGAGA |
| 2232 | CUUUCAAGACUAAUAGAUUGCUCCUUACGAGGAGAC |
| | UCUGCUGGAUGACGUGAGUA |
| 2233 | CUUUCAAGACUAAUAGAUUGCUCCUUACGAGGAGAC |
| | CAUUCUCUGCUGGAUGACGU |
| 2234 | CUUUCAAGACUAAUAGAUUGCUCCUUACGAGGAGAC |
| | CCAUUCUCUGCUGGAUGACG |
| 2235 | CUUUCAAGACUAAUAGAUUGCUCCUUACGAGGAGAC |
| | ACUUUCCAUUCUCUGCUGGA |
| 2236 | CUUUCAAGACUAAUAGAUUGCUCCUUACGAGGAGAC |
| | GACUUUCCAUUCUCUGCUGG |
| 2237 | CUUUCAAGACUAAUAGAUUGCUCCUUACGAGGAGAC |
| | AGGAAAUUUGACUUUCCAUU |
| 2238 | CUUUCAAGACUAAUAGAUUGCUCCUUACGAGGAGAC |
| | CCUGAAUUGCUAUGUGUCUG |
| 2239 | CUUUCAAGACUAAUAGAUUGCUCCUUACGAGGAGAC |
| | CUGAAUUGCUAUGUGUCUGG |
| 2240 | CUUUCAAGACUAAUAGAUUGCUCCUUACGAGGAGAC |
| | CUAUGUGUCUGGGUUUCAUC |
| 2241 | CUUUCAAGACUAAUAGAUUGCUCCUUACGAGGAGAC |
| | AAUGUCGGAUGGAUGAAACC |
| 2242 | CUUUCAAGACUAAUAGAUUGCUCCUUACGAGGAGAC |
| | CAUCCAUCCGACAUUGAAGU |
| 2243 | CUUUCAAGACUAAUAGAUUGCUCCUUACGAGGAGAC |
| | AUCCAUCCGACAUUGAAGUU |
| 2244 | CUUUCAAGACUAAUAGAUUGCUCCUUACGAGGAGAC |
| | AGUAAGUCAACUUCAAUGUC |
| 2245 | CUUUCAAGACUAAUAGAUUGCUCCUUACGAGGAGAC |
| | UUCAGUAAGUCAACUUCAAU |
| 2246 | CUUUCAAGACUAAUAGAUUGCUCCUUACGAGGAGAC |
| | AAGUUGACUUACUGAAGAAU |
| 2247 | CUUUCAAGACUAAUAGAUUGCUCCUUACGAGGAGAC |
| | ACUUACUGAAGAAUGGAGAG |
| 2248 | CUUUCAAGACUAAUAGAUUGCUCCUUACGAGGAGAC |
| | UCUCUCCAUUCUUCAGUAAG |
| 2249 | CUUUCAAGACUAAUAGAUUGCUCCUUACGAGGAGAC |
| | CUGAAGAAUGGAGAGAGAAU |
| 2250 | CUUUCAAGACUAAUAGAUUGCUCCUUACGAGGAGAC |
| | AAUUCUCUCUCCAUUCUUCA |
| 2251 | CUUUCAAGACUAAUAGAUUGCUCCUUACGAGGAGAC |
| | CAAUUCUCUCUCCAUUCUUC |
| 2252 | CUUUCAAGACUAAUAGAUUGCUCCUUACGAGGAGAC |
| | UCAAUUCUCUCUCCAUUCUU |
| 2253 | CUUUCAAGACUAAUAGAUUGCUCCUUACGAGGAGAC |
| | UUCAAUUCUCUCUCCAUUCU |
| 2254 | CUUUCAAGACUAAUAGAUUGCUCCUUACGAGGAGAC |
| | AAAAAGUGGAGCAUUCAGAC |
| 2255 | CUUUCAAGACUAAUAGAUUGCUCCUUACGAGGAGAC |
| | CUGAAAGACAAGUCUGAAUG |
| 2256 | CUUUCAAGACUAAUAGAUUGCUCCUUACGAGGAGAC |
| | AGACUUGUCUUUCAGCAAGG |
| 2257 | CUUUCAAGACUAAUAGAUUGCUCCUUACGAGGAGAC |
| | UCUUUCAGCAAGGACUGGUC |
| 2258 | CUUUCAAGACUAAUAGAUUGCUCCUUACGAGGAGAC |
| | CAGCAAGGACUGGUCUUUCU |
| 2259 | CUUUCAAGACUAAUAGAUUGCUCCUUACGAGGAGAC |
| | AGCAAGGACUGGUCUUUCUA |
| 2260 | CUUUCAAGACUAAUAGAUUGCUCCUUACGAGGAGAC |
| | CUAUCUCUUGUACUACACUG |
| 2261 | CUUUCAAGACUAAUAGAUUGCUCCUUACGAGGAGAC |
| | UAUCUCUUGUACUACACUGA |
| 2262 | CUUUCAAGACUAAUAGAUUGCUCCUUACGAGGAGAC |
| | AGUGUAGUACAAGAGAUAGA |
| 2263 | CUUUCAAGACUAAUAGAUUGCUCCUUACGAGGAGAC |
| | UACUACACUGAAUUCACCCC |
| 2264 | CUUUCAAGACUAAUAGAUUGCUCCUUACGAGGAGAC |
| | AGUGGGGGUGAAUUCAGUGU |
| 2265 | CUUUCAAGACUAAUAGAUUGCUCCUUACGAGGAGAC |
| | CAGUGGGGGUGAAUUCAGUG |
| 2266 | CUUUCAAGACUAAUAGAUUGCUCCUUACGAGGAGAC |
| | UCAGUGGGGGUGAAUUCAGU |
| 2267 | CUUUCAAGACUAAUAGAUUGCUCCUUACGAGGAGAC |
| | UUCAGUGGGGGUGAAUUCAG |
| 2268 | CUUUCAAGACUAAUAGAUUGCUCCUUACGAGGAGAC |
| | ACCCCCACUGAAAAAGAUGA |
| 2269 | CUUUCAAGACUAAUAGAUUGCUCCUUACGAGGAGAC |
| | ACACGGCAGGCAUACUCAUC |
| 2270 | CUUUCAAGACUAAUAGAUUGCUCCUUACGAGGAGAC |
| | GGCUGUGACAAAGUCACAUG |
| 2271 | CUUUCAAGACUAAUAGAUUGCUCCUUACGAGGAGAC |
| | GUCACAGCCCAAGAUAGUUA |
| 2272 | CUUUCAAGACUAAUAGAUUGCUCCUUACGAGGAGAC |
| | UCACAGCCCAAGAUAGUUAA |
| 2273 | CUUUCAAGACUAAUAGAUUGCUCCUUACGAGGAGAC |
| | ACUAUCUUGGGCUGUGACAA |
| 2274 | CUUUCAAGACUAAUAGAUUGCUCCUUACGAGGAGAC |
| | CCCCACUUAACUAUCUUGGG |
| 1381 | CUUUCAAGACUAAUAGAUUGCUCCUUACGAGGAGAC |
| | AGCAAGGACUGGUCUUUCUA |
| 1582 | CUUUCAAGACUAAUAGAUUGCUCCUUACGAGGAGAC |
| | GGGCCGAGAUGUCUCGCUCC |

TABLE 12
CasΦ.32 gRNAs (DNA sequences) targeting human B2M

| | Repeat + spacer RNA Sequence (5′ --> 3′), | SEQ |
|---|---|---|
| Name | shown as DNA | ID NO |
| R3087_CasPhi32 | GCTGGGGACCGATCCTGATTGCTCGCTGCGGCGAG | 987 |
| | ACAATATAAGTGGAGGCGTCGC | |
| R3088_CasPhi32 | GCTGGGGACCGATCCTGATTGCTCGCTGCGGCGAG | 988 |
| | ACATATAAGTGGAGGCGTCGCG | |
| R3089_CasPhi32 | GCTGGGGACCGATCCTGATTGCTCGCTGCGGCGAG | 989 |
| | ACAGGAATGCCCGCCAGCGCGA | |
| R3090_CasPhi32 | GCTGGGGACCGATCCTGATTGCTCGCTGCGGCGAG | 990 |
| | ACCTGAAGCTGACAGCATTCGG | |
| R3091_CasPhi32 | GCTGGGGACCGATCCTGATTGCTCGCTGCGGCGAG | 991 |
| | ACGGGCCGAGATGTCTCGCTCC | |
| R3092_CasPhi32 | GCTGGGGACCGATCCTGATTGCTCGCTGCGGCGAG | 992 |
| | ACGCTGTGCTCGCGCTACTCTC | |
| R3093_CasPhi32 | GCTGGGGACCGATCCTGATTGCTCGCTGCGGCGAG | 993 |
| | ACCTGGCCTGGAGGCTATCCAG | |
| R3094_CasPhi32 | GCTGGGGACCGATCCTGATTGCTCGCTGCGGCGAG | 994 |
| | ACTGGCCTGGAGGCTATCCAGC | |
| R3095_CasPhi32 | GCTGGGGACCGATCCTGATTGCTCGCTGCGGCGAG | 995 |
| | ACATGTGTCTTTTCCCGATATT | |
| R3096_CasPhi32 | GCTGGGGACCGATCCTGATTGCTCGCTGCGGCGAG | 996 |
| | ACTCCCGATATTCCTCAGGTAC | |
| R3097_CasPhi32 | GCTGGGGACCGATCCTGATTGCTCGCTGCGGCGAG | 997 |
| | ACCCCGATATTCCTCAGGTACT | |
| R3098_CasPhi32 | GCTGGGGACCGATCCTGATTGCTCGCTGCGGCGAG | 998 |
| | ACCCGATATTCCTCAGGTACTC | |
| R3099_CasPhi32 | GCTGGGGACCGATCCTGATTGCTCGCTGCGGCGAG | 999 |
| | ACGAGTACCTGAGGAATATCGG | |
| R3100_CasPhi32 | GCTGGGGACCGATCCTGATTGCTCGCTGCGGCGAG | 1000 |
| | ACGGAGTACCTGAGGAATATCG | |
| R3101_CasPhi32 | GCTGGGGACCGATCCTGATTGCTCGCTGCGGCGAG | 1001 |
| | ACCTCAGGTACTCCAAAGATTC | |
| R3102_CasPhi32 | GCTGGGGACCGATCCTGATTGCTCGCTGCGGCGAG | 1002 |
| | ACAGGTTTACTCACGTCATCCA | |
| R3103_CasPhi32 | GCTGGGGACCGATCCTGATTGCTCGCTGCGGCGAG | 1003 |
| | ACACTCACGTCATCCAGCAGAG | |
| R3104_CasPhi32 | GCTGGGGACCGATCCTGATTGCTCGCTGCGGCGAG | 1004 |
| | ACCTCACGTCATCCAGCAGAGA | |
| R3105_CasPhi32 | GCTGGGGACCGATCCTGATTGCTCGCTGCGGCGAG | 1005 |
| | ACTCTGCTGGATGACGTGAGTA | |
| R3106_CasPhi32 | GCTGGGGACCGATCCTGATTGCTCGCTGCGGCGAG | 1006 |
| | ACCATTCTCTGCTGGATGACGT | |
| R3107_CasPhi32 | GCTGGGGACCGATCCTGATTGCTCGCTGCGGCGAG | 1007 |
| | ACCCATTCTCTGCTGGATGACG | |
| R3108_CasPhi32 | GCTGGGGACCGATCCTGATTGCTCGCTGCGGCGAG | 1008 |
| | ACACTTTCCATTCTCTGCTGGA | |
| R3109_CasPhi32 | GCTGGGGACCGATCCTGATTGCTCGCTGCGGCGAG | 1009 |
| | ACGACTTTCCATTCTCTGCTGG | |
| R3110_CasPhi32 | GCTGGGGACCGATCCTGATTGCTCGCTGCGGCGAG | 1010 |
| | ACAGGAAATTTGACTTTCCATT | |
| R3111_CasPhi32 | GCTGGGGACCGATCCTGATTGCTCGCTGCGGCGAG | 1011 |
| | ACCCTGAATTGCTATGTGTCTG | |
| R3112_CasPhi32 | GCTGGGGACCGATCCTGATTGCTCGCTGCGGCGAG | 1012 |
| | ACCTGAATTGCTATGTGTCTGG | |
| R3113_CasPhi32 | GCTGGGGACCGATCCTGATTGCTCGCTGCGGCGAG | 1013 |
| | ACCTATGTGTCTGGGTTTCATC | |
| R3114_CasPhi32 | GCTGGGGACCGATCCTGATTGCTCGCTGCGGCGAG | 1014 |
| | ACAATGTCGGATGGATGAAACC | |
| R3115_CasPhi32 | GCTGGGGACCGATCCTGATTGCTCGCTGCGGCGAG | 1015 |
| | ACCATCCATCCGACATTGAAGT | |
| R3116_CasPhi32 | GCTGGGGACCGATCCTGATTGCTCGCTGCGGCGAG | 1016 |
| | ACATCCATCCGACATTGAAGTT | |
| R3117_CasPhi32 | GCTGGGGACCGATCCTGATTGCTCGCTGCGGCGAG | 1017 |
| | ACAGTAAGTCAACTTCAATGTC | |
| R3118_CasPhi32 | GCTGGGGACCGATCCTGATTGCTCGCTGCGGCGAG | 1018 |
| | ACTTCAGTAAGTCAACTTCAAT | |
| R3119_CasPhi32 | GCTGGGGACCGATCCTGATTGCTCGCTGCGGCGAG | 1019 |
| | ACAAGTTGACTTACTGAAGAAT | |
| R3120_CasPhi32 | GCTGGGGACCGATCCTGATTGCTCGCTGCGGCGAG | 1020 |
| | ACACTTACTGAAGAATGGAGAG | |
| R3121_CasPhi32 | GCTGGGGACCGATCCTGATTGCTCGCTGCGGCGAG | 1021 |
| | ACTCTCTCCATTCTTCAGTAAG | |
| R3122_CasPhi32 | GCTGGGGACCGATCCTGATTGCTCGCTGCGGCGAG | 1022 |
| | ACCTGAAGAATGGAGAGAGAAT | |
| R3123_CasPhi32 | GCTGGGGACCGATCCTGATTGCTCGCTGCGGCGAG | 1023 |
| | ACAATTCTCTCTCCATTCTTCA | |
| R3124_CasPhi32 | GCTGGGGACCGATCCTGATTGCTCGCTGCGGCGAG | 1024 |
| | ACCAATTCTCTCTCCATTCTTC | |
| R3125_CasPhi32 | GCTGGGGACCGATCCTGATTGCTCGCTGCGGCGAG | 1025 |
| | ACTCAATTCTCTCTCCATTCTT | |
| R3126_CasPhi32 | GCTGGGGACCGATCCTGATTGCTCGCTGCGGCGAG | 1026 |
| | ACTTCAATTCTCTCTCCATTCT | |
| R3127_CasPhi32 | GCTGGGGACCGATCCTGATTGCTCGCTGCGGCGAG | 1027 |
| | ACAAAAAGTGGAGCATTCAGAC | |
| R3128_CasPhi32 | GCTGGGGACCGATCCTGATTGCTCGCTGCGGCGAG | 1028 |
| | ACCTGAAAGACAAGTCTGAATG | |
| R3129_CasPhi32 | GCTGGGGACCGATCCTGATTGCTCGCTGCGGCGAG | 1029 |
| | ACAGACTTGTCTTTCAGCAAGG | |
| R3130_CasPhi32 | GCTGGGGACCGATCCTGATTGCTCGCTGCGGCGAG | 1030 |
| | ACTCTTTCAGCAAGGACTGGTC | |
| R3131_CasPhi32 | GCTGGGGACCGATCCTGATTGCTCGCTGCGGCGAG | 1031 |
| | ACCAGCAAGGACTGGTCTTTCT | |
| R3132_CasPhi32 | GCTGGGGACCGATCCTGATTGCTCGCTGCGGCGAG | 1032 |
| | ACAGCAAGGACTGGTCTTTCTA | |
| R3133_CasPhi32 | GCTGGGGACCGATCCTGATTGCTCGCTGCGGCGAG | 1033 |
| | ACCTATCTCTTGTACTACACTG | |
| R3134_CasPhi32 | GCTGGGGACCGATCCTGATTGCTCGCTGCGGCGAG | 1034 |
| | ACTATCTCTTGTACTACACTGA | |
| R3135_CasPhi32 | GCTGGGGACCGATCCTGATTGCTCGCTGCGGCGAG | 1035 |
| | ACAGTGTAGTACAAGAGATAGA | |
| R3148_CasPhi32 | GCTGGGGACCGATCCTGATTGCTCGCTGCGGCGAG | 1036 |
| | ACTACTACACTGAATTCACCCC | |
| R3149_CasPhi32 | GCTGGGGACCGATCCTGATTGCTCGCTGCGGCGAG | 1037 |
| | ACAGTGGGGGTGAATTCAGTGT | |
| R3150_CasPhi32 | GCTGGGGACCGATCCTGATTGCTCGCTGCGGCGAG | 1038 |
| | ACCAGTGGGGGTGAATTCAGTG | |
| R3151_CasPhi32 | GCTGGGGACCGATCCTGATTGCTCGCTGCGGCGAG | 1039 |
| | ACTCAGTGGGGGTGAATTCAGT | |
| R3152_CasPhi32 | GCTGGGGACCGATCCTGATTGCTCGCTGCGGCGAG | 1040 |
| | ACTTCAGTGGGGGTGAATTCAG | |
| R3153_CasPhi32 | GCTGGGGACCGATCCTGATTGCTCGCTGCGGCGAG | 1041 |
| | ACACCCCCACTGAAAAAGATGA | |
| R3154_CasPhi32 | GCTGGGGACCGATCCTGATTGCTCGCTGCGGCGAG | 1042 |
| | ACACACGGCAGGCATACTCATC | |
| R3155_CasPhi32 | GCTGGGGACCGATCCTGATTGCTCGCTGCGGCGAG | 1043 |
| | ACGGCTGTGACAAAGTCACATG | |
| R3156_CasPhi32 | GCTGGGGACCGATCCTGATTGCTCGCTGCGGCGAG | 1044 |
| | ACGTCACAGCCCAAGATAGTTA | |
| R3157_CasPhi32 | GCTGGGGACCGATCCTGATTGCTCGCTGCGGCGAG | 1045 |
| | ACTCACAGCCCAAGATAGTTAA | |
| R3158_CasPhi32 | GCTGGGGACCGATCCTGATTGCTCGCTGCGGCGAG | 1046 |
| | ACACTATCTTGGGCTGTGACAA | |
| R3159_CasPhi32 | GCTGGGGACCGATCCTGATTGCTCGCTGCGGCGAG | 1047 |
| | ACCCCCACTTAACTATCTTGGG | |

TABLE 12.1
CasΦ.32 gRNAs targeting human B2M

| SEQ ID NO | Repeat + spacer RNA Sequence (5′ --> 3′) |
|---|---|
| 2275 | GCUGGGGACCGAUCCUGAUUGCUCGCUGCGGCGAGACAAUAUAAG |
| | UGGAGGCGUCGC |
| 2276 | GCUGGGGACCGAUCCUGAUUGCUCGCUGCGGCGAGACAUAUAAGU |
| | GGAGGCGUCGCG |
| 2277 | GCUGGGGACCGAUCCUGAUUGCUCGCUGCGGCGAGACAGGAAUGCC |
| | CGCCAGCGCGA |
| 2278 | GCUGGGGACCGAUCCUGAUUGCUCGCUGCGGCGAGACCUGAAGCUG |
| | ACAGCAUUCGG |
| 2279 | GCUGGGGACCGAUCCUGAUUGCUCGCUGCGGCGAGACGGGCCGAGA |
| | UGUCUCGCUCC |
| 2280 | GCUGGGGACCGAUCCUGAUUGCUCGCUGCGGCGAGACGCUGUGCUC |
| | GCGCUACUCUC |
| 2281 | GCUGGGGACCGAUCCUGAUUGCUCGCUGCGGCGAGACCUGGCCUGG |
| | AGGCUAUCCAG |
| 2282 | GCUGGGGACCGAUCCUGAUUGCUCGCUGCGGCGAGACUGGCCUGGA |
| | GGCUAUCCAGC |
| 2283 | GCUGGGGACCGAUCCUGAUUGCUCGCUGCGGCGAGACAUGUGUCUU |
| | UUCCCGAUAUU |
| 2284 | GCUGGGGACCGAUCCUGAUUGCUCGCUGCGGCGAGACUCCCGAUAU |
| | UCCUCAGGUAC |
| 2285 | GCUGGGGACCGAUCCUGAUUGCUCGCUGCGGCGAGACCCCGAUAUU |
| | CCUCAGGUACU |
| 2286 | GCUGGGGACCGAUCCUGAUUGCUCGCUGCGGCGAGACCCGAUAUUC |
| | CUCAGGUACUC |
| 2287 | GCUGGGGACCGAUCCUGAUUGCUCGCUGCGGCGAGACGAGUACCUG |
| | AGGAAUAUCGG |
| 2288 | GCUGGGGACCGAUCCUGAUUGCUCGCUGCGGCGAGACGGAGUACCU |
| | GAGGAAUAUCG |
| 2289 | GCUGGGGACCGAUCCUGAUUGCUCGCUGCGGCGAGACCUCAGGUAC |
| | UCCAAAGAUUC |
| 2290 | GCUGGGGACCGAUCCUGAUUGCUCGCUGCGGCGAGACAGGUUUACU |
| | CACGUCAUCCA |
| 2291 | GCUGGGGACCGAUCCUGAUUGCUCGCUGCGGCGAGACACUCACGUC |
| | AUCCAGCAGAG |
| 2292 | GCUGGGGACCGAUCCUGAUUGCUCGCUGCGGCGAGACCUCACGUCA |
| | UCCAGCAGAGA |
| 2293 | GCUGGGGACCGAUCCUGAUUGCUCGCUGCGGCGAGACUCUGCUGGA |
| | UGACGUGAGUA |
| 2294 | GCUGGGGACCGAUCCUGAUUGCUCGCUGCGGCGAGACCAUUCUCUG |
| | CUGGAUGACGU |
| 2295 | GCUGGGGACCGAUCCUGAUUGCUCGCUGCGGCGAGACCCAUUCUCU |
| | GCUGGAUGACG |
| 2296 | GCUGGGGACCGAUCCUGAUUGCUCGCUGCGGCGAGACACUUUCCAU |
| | UCUCUGCUGGA |
| 2297 | GCUGGGGACCGAUCCUGAUUGCUCGCUGCGGCGAGACGACUUUCCA |
| | UUCUCUGCUGG |
| 2298 | GCUGGGGACCGAUCCUGAUUGCUCGCUGCGGCGAGACAGGAAAUU |
| | UGACUUUCCAUU |
| 2299 | GCUGGGGACCGAUCCUGAUUGCUCGCUGCGGCGAGACCCUGAAUUG |
| | CUAUGUGUCUG |
| 2300 | GCUGGGGACCGAUCCUGAUUGCUCGCUGCGGCGAGACCUGAAUUGC |
| | UAUGUGUCUGG |
| 2301 | GCUGGGGACCGAUCCUGAUUGCUCGCUGCGGCGAGACCUAUGUGUC |
| | UGGGUUUCAUC |
| 2302 | GCUGGGGACCGAUCCUGAUUGCUCGCUGCGGCGAGACAAUGUCGGA |
| | UGGAUGAAACC |
| 2303 | GCUGGGGACCGAUCCUGAUUGCUCGCUGCGGCGAGACCAUCCAUCC |
| | GACAUUGAAGU |
| 2304 | GCUGGGGACCGAUCCUGAUUGCUCGCUGCGGCGAGACAUCCAUCCG |
| | ACAUUGAAGUU |
| 2305 | GCUGGGGACCGAUCCUGAUUGCUCGCUGCGGCGAGACAGUAAGUCA |
| | ACUUCAAUGUC |
| 2306 | GCUGGGGACCGAUCCUGAUUGCUCGCUGCGGCGAGACUUCAGUAAG |
| | UCAACUUCAAU |
| 2307 | GCUGGGGACCGAUCCUGAUUGCUCGCUGCGGCGAGACAAGUUGACU |
| | UACUGAAGAAU |
| 2308 | GCUGGGGACCGAUCCUGAUUGCUCGCUGCGGCGAGACACUUACUGA |
| | AGAAUGGAGAG |
| 2309 | GCUGGGGACCGAUCCUGAUUGCUCGCUGCGGCGAGACUCUCUCCAU |
| | UCUUCAGUAAG |
| 2310 | GCUGGGGACCGAUCCUGAUUGCUCGCUGCGGCGAGACCUGAAGAAU |
| | GGAGAGAGAAU |
| 2311 | GCUGGGGACCGAUCCUGAUUGCUCGCUGCGGCGAGACAAUUCUCUC |
| | UCCAUUCUUCA |
| 2312 | GCUGGGGACCGAUCCUGAUUGCUCGCUGCGGCGAGACCAAUUCUCU |
| | CUCCAUUCUUC |
| 2313 | GCUGGGGACCGAUCCUGAUUGCUCGCUGCGGCGAGACUCAAUUCUC |
| | UCUCCAUUCUU |
| 2314 | GCUGGGGACCGAUCCUGAUUGCUCGCUGCGGCGAGACUUCAAUUCU |
| | CUCUCCAUUCU |
| 2315 | GCUGGGGACCGAUCCUGAUUGCUCGCUGCGGCGAGACAAAAAGUG |
| | GAGCAUUCAGAC |
| 2316 | GCUGGGGACCGAUCCUGAUUGCUCGCUGCGGCGAGACCUGAAAGAC |
| | AAGUCUGAAUG |
| 2317 | GCUGGGGACCGAUCCUGAUUGCUCGCUGCGGCGAGACAGACUUGUC |
| | UUUCAGCAAGG |
| 2318 | GCUGGGGACCGAUCCUGAUUGCUCGCUGCGGCGAGACUCUUUCAGC |
| | AAGGACUGGUC |
| 2319 | GCUGGGGACCGAUCCUGAUUGCUCGCUGCGGCGAGACCAGCAAGGA |
| | CUGGUCUUUCU |
| 2320 | GCUGGGGACCGAUCCUGAUUGCUCGCUGCGGCGAGACAGCAAGGAC |
| | UGGUCUUUCUA |
| 2321 | GCUGGGGACCGAUCCUGAUUGCUCGCUGCGGCGAGACCUAUCUCUU |
| | GUACUACACUG |
| 2322 | GCUGGGGACCGAUCCUGAUUGCUCGCUGCGGCGAGACUAUCUCUUG |
| | UACUACACUGA |
| 2323 | GCUGGGGACCGAUCCUGAUUGCUCGCUGCGGCGAGACAGUGUAGU |
| | ACAAGAGAUAGA |
| 2324 | GCUGGGGACCGAUCCUGAUUGCUCGCUGCGGCGAGACUACUACACU |
| | GAAUUCACCCC |
| 2325 | GCUGGGGACCGAUCCUGAUUGCUCGCUGCGGCGAGACAGUGGGGG |
| | UGAAUUCAGUGU |
| 2326 | GCUGGGGACCGAUCCUGAUUGCUCGCUGCGGCGAGACCAGUGGGGG |
| | UGAAUUCAGUG |
| 2327 | GCUGGGGACCGAUCCUGAUUGCUCGCUGCGGCGAGACUCAGUGGGG |
| | GUGAAUUCAGU |
| 2328 | GCUGGGGACCGAUCCUGAUUGCUCGCUGCGGCGAGACUUCAGUGGG |
| | GGUGAAUUCAG |
| 2329 | GCUGGGGACCGAUCCUGAUUGCUCGCUGCGGCGAGACACCCCCACU |
| | GAAAAAGAUGA |
| 2330 | GCUGGGGACCGAUCCUGAUUGCUCGCUGCGGCGAGACACACGGCAG |
| | GCAUACUCAUC |
| 2331 | GCUGGGGACCGAUCCUGAUUGCUCGCUGCGGCGAGACGGCUGUGAC |
| | AAAGUCACAUG |
| 2332 | GCUGGGGACCGAUCCUGAUUGCUCGCUGCGGCGAGACGUCACAGCC |
| | CAAGAUAGUUA |
| 2333 | GCUGGGGACCGAUCCUGAUUGCUCGCUGCGGCGAGACUCACAGCCC |
| | AAGAUAGUUAA |
| 2334 | GCUGGGGACCGAUCCUGAUUGCUCGCUGCGGCGAGACACUAUCUUG |
| | GGCUGUGACAA |
| 2335 | GCUGGGGACCGAUCCUGAUUGCUCGCUGCGGCGAGACCCCCACUUA |
| | ACUAUCUUGGG |

TABLE 13
CasΦ.32 gRNAs targeting human CIITA

| | Repeat + spacer sequence RNA | SEQ ID |
|---|---|---|
| Name | Sequence (5′ --> 3′) | NO |
| R4503_CasPhi32_C2TA_T1.1 | GCUGGGGACCGAUCCUGAUUGCUCGCUGCGGCGAGA | 1048 |
| | CCUACACAAUGCGUUGCCUGG | |
| R4504_CasPhi32_C2TA_T1.2 | GCUGGGGACCGAUCCUGAUUGCUCGCUGCGGCGAGA | 1049 |
| | CGGGCUCUGACAGGUAGGACC | |
| R4505_CasPhi32_C2TA_T1.3 | GCUGGGGACCGAUCCUGAUUGCUCGCUGCGGCGAGA | 1050 |
| | CUGUAGGAAUCCCAGCCAGGC | |
| R4506_CasPhi32_C2TA_T1.8 | GCUGGGGACCGAUCCUGAUUGCUCGCUGCGGCGAGA | 1051 |
| | CCCUGGCUCCACGCCCUGCUG | |
| R4507_CasPhi32_C2TA_T1.9 | GCUGGGGACCGAUCCUGAUUGCUCGCUGCGGCGAGA | 1052 |
| | CGGGAAGCUGAGGGCACGAGG | |
| R4508_CasPhi32_C2TA_T2.1 | GCUGGGGACCGAUCCUGAUUGCUCGCUGCGGCGAGA | 1053 |
| | CACAGCGAUGCUGACCCCCUG | |
| R4509_CasPhi32_C2TA_T2.2 | GCUGGGGACCGAUCCUGAUUGCUCGCUGCGGCGAGA | 1054 |
| | CUUAACAGCGAUGCUGACCCC | |
| R4510_CasPhi32_C2TA_T2.3 | GCUGGGGACCGAUCCUGAUUGCUCGCUGCGGCGAGA | 1055 |
| | CUAUGACCAGAUGGACCUGGC | |
| R4511_CasPhi32_C2TA_T2.4 | GCUGGGGACCGAUCCUGAUUGCUCGCUGCGGCGAGA | 1056 |
| | CGGGCCCCUAGAAGGUGGCUA | |
| R4512_CasPhi32_C2TA_T2.5 | GCUGGGGACCGAUCCUGAUUGCUCGCUGCGGCGAGA | 1057 |
| | CUAGGGGCCCCAACUCCAUGG | |
| R4513_CasPhi32_C2TA_T2.6 | GCUGGGGACCGAUCCUGAUUGCUCGCUGCGGCGAGA | 1058 |
| | CAGAAGCUCCAGGUAGCCACC | |
| R4514_CasPhi32_C2TA_T2.7 | GCUGGGGACCGAUCCUGAUUGCUCGCUGCGGCGAGA | 1059 |
| | CUCCAGCCAGGUCCAUCUGGU | |
| R4515_CasPhi32_C2TA_T2.8 | GCUGGGGACCGAUCCUGAUUGCUCGCUGCGGCGAGA | 1060 |
| | CUUCUCCAGCCAGGUCCAUCU | |

TABLE 14
Shortened CasΦ.12 gRNAs (DNA sequences) targeting human TRAC

| | | SEQ |
|---|---|---|
| | Repeat + spacer RNA Sequence (5′ --> 3′), | ID |
| Name | shown as DNA | NO |
| R3040_CasPhi12_S | ATTGCTCCTTACGAGGAGACTGGATATCTGTGGGACA | 1061 |
| R3041_CasPhi12_S | ATTGCTCCTTACGAGGAGACTCCCACAGATATCCAGA | 1062 |
| R3042_CasPhi12 S | ATTGCTCCTTACGAGGAGACGAGTCTCTCAGCTGGTA | 1063 |
| R3043_CasPhi12_S | ATTGCTCCTTACGAGGAGACAGAGTCTCTCAGCTGGT | 1064 |
| R3044_CasPhi12_S | ATTGCTCCTTACGAGGAGACTCACTGGATTTAGAGTC | 1065 |
| R3045_CasPhi12_S | ATTGCTCCTTACGAGGAGACAGAATCAAAATCGGTGA | 1066 |
| R3046_CasPhi12_S | ATTGCTCCTTACGAGGAGACGAGAATCAAAATCGGTG | 1067 |
| R3047_CasPhi12_S | ATTGCTCCTTACGAGGAGACACCGATTTTGATTCTCA | 1068 |
| R3048_CasPhi12_S | ATTGCTCCTTACGAGGAGACTTTGAGAATCAAAATCG | 1069 |
| R3049_CasPhi12 S | ATTGCTCCTTACGAGGAGACGTTTGAGAATCAAAATC | 1070 |
| R3050_CasPhi12_S | ATTGCTCCTTACGAGGAGACTGATTCTCAAACAAATG | 1071 |
| R3051_CasPhi12_S | ATTGCTCCTTACGAGGAGACGATTCTCAAACAAATGT | 1072 |
| R3052_CasPhi12_S | ATTGCTCCTTACGAGGAGACATTCTCAAACAAATGTG | 1073 |
| R3053_CasPhi12_S | ATTGCTCCTTACGAGGAGACTGACACATTTGTTTGAG | 1074 |
| R3054_CasPhi12_S | ATTGCTCCTTACGAGGAGACTCAAACAAATGTGTCAC | 1075 |
| R3055_CasPhi12_S | ATTGCTCCTTACGAGGAGACGTGACACATTTGTTTGA | 1076 |
| R3056_CasPhi12_S | ATTGCTCCTTACGAGGAGACCTTTGTGACACATTTGT | 1077 |
| R3057_CasPhi12_S | ATTGCTCCTTACGAGGAGACTGATGTGTATATCACAG | 1078 |
| R3058_CasPhi12_S | ATTGCTCCTTACGAGGAGACTCTGTGATATACACATC | 1079 |
| R3059_CasPhi12_S | ATTGCTCCTTACGAGGAGACGTCTGTGATATACACAT | 1080 |
| R3060_CasPhi12_S | ATTGCTCCTTACGAGGAGACTGTCTGTGATATACACA | 1081 |
| R3061_CasPhi12_S | ATTGCTCCTTACGAGGAGACAAGTCCATAGACCTCAT | 1082 |
| R3062_CasPhi12_S | ATTGCTCCTTACGAGGAGACCTCTTGAAGTCCATAGA | 1083 |
| R3063_CasPhi12 S | ATTGCTCCTTACGAGGAGACAAGAGCAACAGTGCTGT | 1084 |
| R3064_CasPhi12_S | ATTGCTCCTTACGAGGAGACCTCCAGGCCACAGCACT | 1085 |
| R3065_CasPhi12_S | ATTGCTCCTTACGAGGAGACTTGCTCCAGGCCACAGC | 1086 |
| R3066_CasPhi12_S | ATTGCTCCTTACGAGGAGACGTTGCTCCAGGCCACAG | 1087 |
| R3067_CasPhi12_S | ATTGCTCCTTACGAGGAGACCACATGCAAAGTCAGAT | 1088 |
| R3068_CasPhi12_S | ATTGCTCCTTACGAGGAGACGCACATGCAAAGTCAGA | 1089 |
| R3069_CasPhi12_S | ATTGCTCCTTACGAGGAGACGCATGTGCAAACGCCTT | 1090 |
| R3070_CasPhi12_S | ATTGCTCCTTACGAGGAGACAAGGCGTTTGCACATGC | 1091 |
| R3071_CasPhi12_S | ATTGCTCCTTACGAGGAGACCATGTGCAAACGCCTTC | 1092 |
| R3072_CasPhi12_S | ATTGCTCCTTACGAGGAGACTTGAAGGCGTTTGCACA | 1093 |
| R3073_CasPhi12_S | ATTGCTCCTTACGAGGAGACAACAACAGCATTATTCC | 1094 |
| R3074_CasPhi12_S | ATTGCTCCTTACGAGGAGACTGGAATAATGCTGTTGT | 1095 |
| R3075_CasPhi12_S | ATTGCTCCTTACGAGGAGACTTCCAGAAGACACCTTC | 1096 |
| R3076_CasPhi12_S | ATTGCTCCTTACGAGGAGACCAGAAGACACCTTCTTC | 1097 |
| R3077_CasPhi12_S | ATTGCTCCTTACGAGGAGACCCTGGGCTGGGGAAGAA | 1098 |
| R3078_CasPhi12_S | ATTGCTCCTTACGAGGAGACTTCCCCAGCCCAGGTAA | 1099 |
| R3079_CasPhi12_S | ATTGCTCCTTACGAGGAGACCCCAGCCCAGGTAAGGG | 1100 |
| R3080_CasPhi12_S | ATTGCTCCTTACGAGGAGACTAAAAGGAAAAACAGA | 1101 |
| | C | |
| R3081_CasPhi12_S | ATTGCTCCTTACGAGGAGACCTAAAAGGAAAAACAG | 1102 |
| | A | |
| R3082_CasPhi12_S | ATTGCTCCTTACGAGGAGACTTCCTTTTAGAAAGTTC | 1103 |
| R3083_CasPhi12_S | ATTGCTCCTTACGAGGAGACTCCTTTTAGAAAGTTCC | 1104 |
| R3084_CasPhi12 S | ATTGCTCCTTACGAGGAGACCCTTTTAGAAAGTTCCT | 1105 |
| R3085_CasPhi12_S | ATTGCTCCTTACGAGGAGACCTTTTAGAAAGTTCCTG | 1106 |
| R3086_CasPhi12_S | ATTGCTCCTTACGAGGAGACTAGAAAGTTCCTGTGAT | 1107 |
| R3136_CasPhi12_S | ATTGCTCCTTACGAGGAGACAGAAAGTTCCTGTGATG | 1108 |
| R3137_CasPhi12_S | ATTGCTCCTTACGAGGAGACGAAAGTTCCTGTGATGT | 1109 |
| R3138_CasPhi12_S | ATTGCTCCTTACGAGGAGACACATCACAGGAACTTTC | 1110 |
| R3139_CasPhi12 S | ATTGCTCCTTACGAGGAGACCTGTGATGTCAAGCTGG | 1111 |
| R3140_CasPhi12_S | ATTGCTCCTTACGAGGAGACTCGACCAGCTTGACATC | 1112 |
| R3141_CasPhi12_S | ATTGCTCCTTACGAGGAGACCTCGACCAGCTTGACAT | 1113 |
| R3142_CasPhi12_S | ATTGCTCCTTACGAGGAGACTCTCGACCAGCTTGACA | 1114 |
| R3143_CasPhi12_S | ATTGCTCCTTACGAGGAGACAAAGCTTTTCTCGACCA | 1115 |
| R3144_CasPhi12_S | ATTGCTCCTTACGAGGAGACCAAAGCTTTTCTCGACC | 1116 |
| R3145_CasPhi12_S | ATTGCTCCTTACGAGGAGACCCTGTTTCAAAGCTTTT | 1117 |
| R3146_CasPhi12_S | ATTGCTCCTTACGAGGAGACGAAACAGGTAAGACAG | 1118 |
| | G | |
| R3147_CasPhi12_S | ATTGCTCCTTACGAGGAGACAAACAGGTAAGACAGG | 1119 |
| | G | |

TABLE 14.1
Shortened_CasΦ.12 gRNAs targeting human TRAC

| SEQ ID NO | Repeat + spacer RNA Sequence (5′ --> 3′) |
|---|---|
| 2370 | AUUGCUCCUUACGAGGAGACUGGAUAUCUGUGGGACA |
| 2371 | AUUGCUCCUUACGAGGAGACUCCCACAGAUAUCCAGA |
| 2372 | AUUGCUCCUUACGAGGAGACGAGUCUCUCAGCUGGUA |
| 2373 | AUUGCUCCUUACGAGGAGACAGAGUCUCUCAGCUGGU |
| 2374 | AUUGCUCCUUACGAGGAGACUCACUGGAUUUAGAGUC |
| 2375 | AUUGCUCCUUACGAGGAGACAGAAUCAAAAUCGGUGA |
| 2376 | AUUGCUCCUUACGAGGAGACGAGAAUCAAAAUCGGUG |
| 2377 | AUUGCUCCUUACGAGGAGACACCGAUUUUGAUUCUCA |
| 2378 | AUUGCUCCUUACGAGGAGACUUUGAGAAUCAAAAUCG |
| 2379 | AUUGCUCCUUACGAGGAGACGUUUGAGAAUCAAAAUC |
| 2380 | AUUGCUCCUUACGAGGAGACUGAUUCUCAAACAAAUG |
| 2381 | AUUGCUCCUUACGAGGAGACGAUUCUCAAACAAAUGU |
| 2382 | AUUGCUCCUUACGAGGAGACAUUCUCAAACAAAUGUG |
| 2383 | AUUGCUCCUUACGAGGAGACUGACACAUUUGUUUGAG |
| 2384 | AUUGCUCCUUACGAGGAGACUCAAACAAAUGUGUCAC |
| 2385 | AUUGCUCCUUACGAGGAGACGUGACACAUUUGUUUGA |
| 2386 | AUUGCUCCUUACGAGGAGACCUUUGUGACACAUUUGU |
| 2387 | AUUGCUCCUUACGAGGAGACUGAUGUGUAUAUCACAG |
| 2388 | AUUGCUCCUUACGAGGAGACUCUGUGAUAUACACAUC |
| 2389 | AUUGCUCCUUACGAGGAGACGUCUGUGAUAUACACAU |
| 2390 | AUUGCUCCUUACGAGGAGACUGUCUGUGAUAUACACA |
| 2391 | AUUGCUCCUUACGAGGAGACAAGUCCAUAGACCUCAU |
| 2392 | AUUGCUCCUUACGAGGAGACCUCUUGAAGUCCAUAGA |
| 2393 | AUUGCUCCUUACGAGGAGACAAGAGCAACAGUGCUGU |
| 2394 | AUUGCUCCUUACGAGGAGACCUCCAGGCCACAGCACU |
| 2395 | AUUGCUCCUUACGAGGAGACUUGCUCCAGGCCACAGC |
| 2396 | AUUGCUCCUUACGAGGAGACGUUGCUCCAGGCCACAG |
| 2397 | AUUGCUCCUUACGAGGAGACCACAUGCAAAGUCAGAU |
| 2398 | AUUGCUCCUUACGAGGAGACGCACAUGCAAAGUCAGA |
| 2399 | AUUGCUCCUUACGAGGAGACGCAUGUGCAAACGCCUU |
| 2400 | AUUGCUCCUUACGAGGAGACAAGGCGUUUGCACAUGC |
| 2401 | AUUGCUCCUUACGAGGAGACCAUGUGCAAACGCCUUC |
| 2402 | AUUGCUCCUUACGAGGAGACUUGAAGGCGUUUGCACA |
| 2403 | AUUGCUCCUUACGAGGAGACAACAACAGCAUUAUUCC |
| 2404 | AUUGCUCCUUACGAGGAGACUGGAAUAAUGCUGUUGU |
| 2405 | AUUGCUCCUUACGAGGAGACUUCCAGAAGACACCUUC |
| 2406 | AUUGCUCCUUACGAGGAGACCAGAAGACACCUUCUUC |
| 2407 | AUUGCUCCUUACGAGGAGACCCUGGGCUGGGGAAGAA |
| 2408 | AUUGCUCCUUACGAGGAGACUUCCCCAGCCCAGGUAA |
| 2409 | AUUGCUCCUUACGAGGAGACCCCAGCCCAGGUAAGGG |
| 2410 | AUUGCUCCUUACGAGGAGACUAAAAGGAAAAACAGAC |
| 2411 | AUUGCUCCUUACGAGGAGACCUAAAAGGAAAAACAGA |
| 2412 | AUUGCUCCUUACGAGGAGACUUCCUUUUAGAAAGUUC |
| 2413 | AUUGCUCCUUACGAGGAGACUCCUUUUAGAAAGUUCC |
| 2414 | AUUGCUCCUUACGAGGAGACCCUUUUAGAAAGUUCCU |
| 2415 | AUUGCUCCUUACGAGGAGACCUUUUAGAAAGUUCCUG |
| 2416 | AUUGCUCCUUACGAGGAGACUAGAAAGUUCCUGUGAU |
| 2417 | AUUGCUCCUUACGAGGAGACAGAAAGUUCCUGUGAUG |
| 2418 | AUUGCUCCUUACGAGGAGACGAAAGUUCCUGUGAUGU |
| 2419 | AUUGCUCCUUACGAGGAGACACAUCACAGGAACUUUC |
| 2420 | AUUGCUCCUUACGAGGAGACCUGUGAUGUCAAGCUGG |
| 2421 | AUUGCUCCUUACGAGGAGACUCGACCAGCUUGACAUC |
| 2422 | AUUGCUCCUUACGAGGAGACCUCGACCAGCUUGACAU |
| 2423 | AUUGCUCCUUACGAGGAGACUCUCGACCAGCUUGACA |
| 2424 | AUUGCUCCUUACGAGGAGACAAAGCUUUUCUCGACCA |
| 2425 | AUUGCUCCUUACGAGGAGACCAAAGCUUUUCUCGACC |
| 2426 | AUUGCUCCUUACGAGGAGACCCUGUUUCAAAGCUUUU |
| 2427 | AUUGCUCCUUACGAGGAGACGAAACAGGUAAGACAGG |
| 2428 | AUUGCUCCUUACGAGGAGACAAACAGGUAAGACAGGG |
| 1354 | AUUGCUCCUUACGAGGAGACGAGUCUCUCAGCUGGUACAC |
| 1357 | AUUGCUCCUUACGAGGAGACGAGUCUCUCAGCUGGUA |

TABLE 15
Shortened CasΦ.12 gRNAs (DNA sequences) targeting human B2M

| | Repeat + spacer RNA Sequence (5′ --> 3′), | SEQ ID |
|---|---|---|
| Name | shown as DNA | NO |
| R3115_CasPhi12_S | ATTGCTCCTTACGAGGAGACCATCCATCCGACATTGA | 1120 |
| R3116_CasPhi12_S | ATTGCTCCTTACGAGGAGACATCCATCCGACATTGAA | 1121 |
| R3117_CasPhi12_S | ATTGCTCCTTACGAGGAGACAGTAAGTCAACTTCAAT | 1122 |
| R3118_CasPhi12_S | ATTGCTCCTTACGAGGAGACTTCAGTAAGTCAACTTC | 1123 |
| R3119_CasPhi12_S | ATTGCTCCTTACGAGGAGACAAGTTGACTTACTGAAG | 1124 |
| R3120_CasPhi12_S | ATTGCTCCTTACGAGGAGACACTTACTGAAGAATGGA | 1125 |
| R3121_CasPhi12_S | ATTGCTCCTTACGAGGAGACTCTCTCCATTCTTCAGT | 1126 |
| R3122_CasPhi12_S | ATTGCTCCTTACGAGGAGACCTGAAGAATGGAGAGAG | 1127 |
| R3123_CasPhi12_S | ATTGCTCCTTACGAGGAGACAATTCTCTCTCCATTCT | 1128 |
| R3124_CasPhi12_S | ATTGCTCCTTACGAGGAGACCAATTCTCTCTCCATTC | 1129 |
| R3125_CasPhi12_S | ATTGCTCCTTACGAGGAGACTCAATTCTCTCTCCATT | 1130 |
| R3126_CasPhi12_S | ATTGCTCCTTACGAGGAGACTTCAATTCTCTCTCCAT | 1131 |
| R3127_CasPhi12_S | ATTGCTCCTTACGAGGAGACAAAAAGTGGAGCATTCA | 1132 |
| R3128_CasPhi12_S | ATTGCTCCTTACGAGGAGACCTGAAAGACAAGTCTGA | 1133 |
| R3129_CasPhi12_S | ATTGCTCCTTACGAGGAGACAGACTTGTCTTTCAGCA | 1134 |
| R3130_CasPhi12_S | ATTGCTCCTTACGAGGAGACTCTTTCAGCAAGGACTG | 1135 |
| R3131_CasPhi12_S | ATTGCTCCTTACGAGGAGACCAGCAAGGACTGGTCTT | 1136 |
| R3132_CasPhi12_S | ATTGCTCCTTACGAGGAGACAGCAAGGACTGGTCTTT | 1137 |
| R3133_CasPhi12_S | ATTGCTCCTTACGAGGAGACCTATCTCTTGTACTACA | 1138 |
| R3134_CasPhi12_S | ATTGCTCCTTACGAGGAGACTATCTCTTGTACTACAC | 1139 |
| R3135_CasPhi12_S | ATTGCTCCTTACGAGGAGACAGTGTAGTACAAGAGAT | 1140 |
| R3148_CasPhi12_S | ATTGCTCCTTACGAGGAGACTACTACACTGAATTCAC | 1141 |
| R3149_CasPhil2_S | ATTGCTCCTTACGAGGAGACAGTGGGGGTGAATTCAG | 1142 |
| R3150_CasPhi12_S | ATTGCTCCTTACGAGGAGACCAGTGGGGGTGAATTCA | 1143 |
| R3151_CasPhi12_S | ATTGCTCCTTACGAGGAGACTCAGTGGGGGTGAATTC | 1144 |
| R3152_CasPhi12_S | ATTGCTCCTTACGAGGAGACTTCAGTGGGGGTGAATT | 1145 |
| R3153_CasPhi12_S | ATTGCTCCTTACGAGGAGACACCCCCACTGAAAAAGA | 1146 |
| R3154_CasPhi12_S | ATTGCTCCTTACGAGGAGACACACGGCAGGCATACTC | 1147 |
| R3155_CasPhi12_S | ATTGCTCCTTACGAGGAGACGGCTGTGACAAAGTCAC | 1148 |
| R3156_CasPhi12_S | ATTGCTCCTTACGAGGAGACGTCACAGCCCAAGATAG | 1149 |
| R3157_CasPhil2_S | ATTGCTCCTTACGAGGAGACTCACAGCCCAAGATAGT | 1150 |
| R3158_CasPhi12_S | ATTGCTCCTTACGAGGAGACACTATCTTGGGCTGTGA | 1151 |
| R3159_CasPhi12_S | ATTGCTCCTTACGAGGAGACCCCCACTTAACTATCTT | 1152 |

TABLE 15.1
Shortened CasΦ.12 gRNAs targeting human B2M

| SEQ ID | |
|---|---|
| NO | Repeat + spacer RNA Sequence (5′ --> 3′) |
| 2337 | AUUGCUCCUUACGAGGAGACCAUCCAUCCGACAUUGA |
| 2338 | AUUGCUCCUUACGAGGAGACAUCCAUCCGACAUUGAA |
| 2339 | AUUGCUCCUUACGAGGAGACAGUAAGUCAACUUCAAU |
| 2340 | AUUGCUCCUUACGAGGAGACUUCAGUAAGUCAACUUC |
| 2341 | AUUGCUCCUUACGAGGAGACAAGUUGACUUACUGAAG |
| 2342 | AUUGCUCCUUACGAGGAGACACUUACUGAAGAAUGGA |
| 2343 | AUUGCUCCUUACGAGGAGACUCUCUCCAUUCUUCAGU |
| 2344 | AUUGCUCCUUACGAGGAGACCUGAAGAAUGGAGAGAG |
| 2345 | AUUGCUCCUUACGAGGAGACAAUUCUCUCUCCAUUCU |
| 2346 | AUUGCUCCUUACGAGGAGACCAAUUCUCUCUCCAUUC |
| 2347 | AUUGCUCCUUACGAGGAGACUCAAUUCUCUCUCCAUU |
| 2348 | AUUGCUCCUUACGAGGAGACUUCAAUUCUCUCUCCAU |
| 2349 | AUUGCUCCUUACGAGGAGACAAAAAGUGGAGCAUUCA |
| 2350 | AUUGCUCCUUACGAGGAGACCUGAAAGACAAGUCUGA |
| 2351 | AUUGCUCCUUACGAGGAGACAGACUUGUCUUUCAGCA |
| 2352 | AUUGCUCCUUACGAGGAGACUCUUUCAGCAAGGACUG |
| 2353 | AUUGCUCCUUACGAGGAGACCAGCAAGGACUGGUCUU |
| 2354 | AUUGCUCCUUACGAGGAGACAGCAAGGACUGGUCUUU |
| 2355 | AUUGCUCCUUACGAGGAGACCUAUCUCUUGUACUACA |
| 2356 | AUUGCUCCUUACGAGGAGACUAUCUCUUGUACUACAC |
| 2357 | AUUGCUCCUUACGAGGAGACAGUGUAGUACAAGAGAU |
| 2358 | AUUGCUCCUUACGAGGAGACUACUACACUGAAUUCAC |
| 2359 | AUUGCUCCUUACGAGGAGACAGUGGGGGUGAAUUCAG |
| 2360 | AUUGCUCCUUACGAGGAGACCAGUGGGGGUGAAUUCA |
| 2361 | AUUGCUCCUUACGAGGAGACUCAGUGGGGGUGAAUUC |
| 2362 | AUUGCUCCUUACGAGGAGACUUCAGUGGGGGUGAAUU |
| 2363 | AUUGCUCCUUACGAGGAGACACCCCCACUGAAAAAGA |
| 2364 | AUUGCUCCUUACGAGGAGACACACGGCAGGCAUACUC |
| 2365 | AUUGCUCCUUACGAGGAGACGGCUGUGACAAAGUCAC |
| 2366 | AUUGCUCCUUACGAGGAGACGUCACAGCCCAAGAUAG |
| 2367 | AUUGCUCCUUACGAGGAGACUCACAGCCCAAGAUAGU |
| 2368 | AUUGCUCCUUACGAGGAGACACUAUCUUGGGCUGUGA |
| 2369 | AUUGCUCCUUACGAGGAGACCCCCACUUAACUAUCUU |

TABLE 16
Shortened CasΦ.12 gRNAs targeting human CIITA

| | | SEQ ID |
|---|---|---|
| Name | Repeat + spacer RNA Sequence (5′ --> 3′) | NO |
| R4503_CasPhi12 | AUUGCUCCUUACGAGGAGACCUACACAAUGCGUUGCC | 1153 |
| C2TA_T1.1_S | | |
| R4504_CasPhi12 | AUUGCUCCUUACGAGGAGACGGGCUCUGACAGGUAGG | 1154 |
| C2TA_T1.2_S | | |
| R4505_CasPhi12 | AUUGCUCCUUACGAGGAGACUGUAGGAAUCCCAGCCA | 1155 |
| C2TA_T1.3_S | | |
| R4506_CasPhi12 | AUUGCUCCUUACGAGGAGACCCUGGCUCCACGCCCUG | 1156 |
| C2TA_T1.8_S | | |
| R4507_CasPhi12 | AUUGCUCCUUACGAGGAGACGGGAAGCUGAGGGCACG | 1157 |
| C2TA_T1.9_S | | |
| R4508_CasPhi12 | AUUGCUCCUUACGAGGAGACACAGCGAUGCUGACCCC | 1158 |
| C2TA_T2.1_S | | |
| R4509_CasPhi12 | AUUGCUCCUUACGAGGAGACUUAACAGCGAUGCUGAC | 1159 |
| C2TA_T2.2_S | | |
| R4510_CasPhi12 | AUUGCUCCUUACGAGGAGACUAUGACCAGAUGGACCU | 1160 |
| C2TA_T2.3_S | | |
| R4511_CasPhi12 | AUUGCUCCUUACGAGGAGACGGGCCCCUAGAAGGUGG | 1161 |
| C2TA_T2.4_S | | |
| R4512_CasPhi12 | AUUGCUCCUUACGAGGAGACUAGGGGCCCCAACUCCA | 1162 |
| C2TA_T2.5_S | | |
| R4513_CasPhi12 | AUUGCUCCUUACGAGGAGACAGAAGCUCCAGGUAGCC | 1163 |
| C2TA_T2.6_S | | |
| R4514_CasPhi12 | AUUGCUCCUUACGAGGAGACUCCAGCCAGGUCCAUCU | 1164 |
| C2TA_T2.7_S | | |
| R4515_CasPhi12 | AUUGCUCCUUACGAGGAGACUUCUCCAGCCAGGUCCA | 1165 |
| C2TA_T2.8_S | | |
| R5200_CasPhi12_S | AUUGCUCCUUACGAGGAGACAGCAGGCUGUUGUGUGA | 1166 |
| R5201_CasPhil2_S | AUUGCUCCUUACGAGGAGACCAUGUCACACAACAGCC | 1167 |
| R5202_CasPhi12_S | AUUGCUCCUUACGAGGAGACUGUGACAUGGAAGGUGA | 1168 |
| R5203_CasPhi12_S | AUUGCUCCUUACGAGGAGACAUCACCUUCCAUGUCAC | 1169 |
| R5204_CasPhil2_S | AUUGCUCCUUACGAGGAGACGCAUAAGCCUCCCUGGU | 1170 |
| R5205_CasPhi12_S | AUUGCUCCUUACGAGGAGACCAGGACUCCCAGCUGGA | 1171 |
| R5206_CasPhil2_S | AUUGCUCCUUACGAGGAGACCUCAGGCCCUCCAGCUG | 1172 |
| R5207_CasPhi12_S | AUUGCUCCUUACGAGGAGACUGCUGGCAUCUCCAUAC | 1173 |
| R5208_CasPhi12_S | AUUGCUCCUUACGAGGAGACUGCCCAACUUCUGCUGG | 1174 |
| R5209_CasPhi12_S | AUUGCUCCUUACGAGGAGACCUGCCCAACUUCUGCUG | 1175 |
| R5210_CasPhi12_S | AUUGCUCCUUACGAGGAGACUCUGCCCAACUUCUGCU | 1176 |
| R5211_CasPhi12_S | AUUGCUCCUUACGAGGAGACUGACUUUUCUGCCCAAC | 1177 |
| R5212_CasPhi12_S | AUUGCUCCUUACGAGGAGACCUGACUUUUCUGCCCAA | 1178 |
| R5213_CasPhi12_S | AUUGCUCCUUACGAGGAGACUCUGACUUUUCUGCCCA | 1179 |
| R5214_CasPhi12_S | AUUGCUCCUUACGAGGAGACCCAGAGGAGCUUCCGGC | 1180 |
| R5215_CasPhi12_S | AUUGCUCCUUACGAGGAGACAGGUCUGCCGGAAGCUC | 1181 |
| R5216_CasPhil2_S | AUUGCUCCUUACGAGGAGACCGGCAGACCUGAAGCAC | 1182 |
| R5217_CasPhi12_S | AUUGCUCCUUACGAGGAGACCAGUGCUUCAGGUCUGC | 1183 |
| R5218_CasPhi12_S | AUUGCUCCUUACGAGGAGACAACAGCGCAGGCAGUGG | 1184 |
| R5219_CasPhil2_S | AUUGCUCCUUACGAGGAGACAACCAGGAGCCAGCCUC | 1185 |
| R5220_CasPhi12_S | AUUGCUCCUUACGAGGAGACUCCAGGCGCAUCUGGCC | 1186 |
| R5221_CasPhi12_S | AUUGCUCCUUACGAGGAGACCUCCAGGCGCAUCUGGC | 1187 |
| R5222_CasPhi12_S | AUUGCUCCUUACGAGGAGACUCUCCAGGCGCAUCUGG | 1188 |
| R5223_CasPhi12_S | AUUGCUCCUUACGAGGAGACCUCCAGUUCCUCGUUGA | 1189 |
| R5224_CasPhi12_S | AUUGCUCCUUACGAGGAGACUCCAGUUCCUCGUUGAG | 1190 |
| R5225_CasPhi12_S | AUUGCUCCUUACGAGGAGACAGGCAGCUCAACGAGGA | 1191 |
| R5226_CasPhi12_S | AUUGCUCCUUACGAGGAGACCUCGUUGAGCUGCCUGA | 1192 |
| R5227_CasPhi12_S | AUUGCUCCUUACGAGGAGACAGCUGCCUGAAUCUCCC | 1193 |
| R5228_CasPhi12_S | AUUGCUCCUUACGAGGAGACGUCCCCACCAUCUCCAC | 1194 |
| R5229_CasPhi12_S | AUUGCUCCUUACGAGGAGACUCCCCACCAUCUCCACU | 1195 |
| R5230_CasPhi12_S | AUUGCUCCUUACGAGGAGACCCAGAGCCCAUGGGGCA | 1196 |
| R5231_CasPhi12_S | AUUGCUCCUUACGAGGAGACGCCAGAGCCCAUGGGGC | 1197 |
| R5232_CasPhi12_S | AUUGCUCCUUACGAGGAGACCAGCCUCAGAGAUUUGC | 1198 |
| R5233_CasPhi12_S | AUUGCUCCUUACGAGGAGACGGAGGCCGUGGACAGUG | 1199 |
| R5234_CasPhi12_S | AUUGCUCCUUACGAGGAGACACUGUCCACGGCCUCCC | 1200 |
| R5235_CasPhi12_S | AUUGCUCCUUACGAGGAGACGCUCCAUCAGCCACUGA | 1201 |
| R5236_CasPhi12_S | AUUGCUCCUUACGAGGAGACAGGCAUGCUGGGCAGGU | 1202 |
| R5237_CasPhi12_S | AUUGCUCCUUACGAGGAGACCUCGGGAGGUCAGGGCA | 1203 |
| R5238_CasPhi12_S | AUUGCUCCUUACGAGGAGACGCUCGGGAGGUCAGGGC | 1204 |
| R5239_CasPhi12_S | AUUGCUCCUUACGAGGAGACGAGACCUCUCCAGCUGC | 1205 |
| R5240_CasPhi12_S | AUUGCUCCUUACGAGGAGACUUGGAGACCUCUCCAGC | 1206 |
| R5241_CasPhi12_S | AUUGCUCCUUACGAGGAGACGAAGCUUGUUGGAGACC | 1207 |
| R5242_CasPhi12_S | AUUGCUCCUUACGAGGAGACGGAAGCUUGUUGGAGAC | 1208 |
| R5243_CasPhi12_S | AUUGCUCCUUACGAGGAGACUGGAAGCUUGUUGGAGA | 1209 |
| R5244_CasPhi12_S | AUUGCUCCUUACGAGGAGACUACCGCUCACUGCAGGA | 1210 |
| R5245_CasPhi12_S | AUUGCUCCUUACGAGGAGACCUGCUGCUCCUCUCCAG | 1211 |
| R5246_CasPhi12_S | AUUGCUCCUUACGAGGAGACCCGCUCCAGGCUCUUGC | 1212 |
| R5247_CasPhi12_S | AUUGCUCCUUACGAGGAGACUGCCCAGUCCGGGGUGG | 1213 |
| R5248_CasPhi12_S | AUUGCUCCUUACGAGGAGACGGCCAGCUGCCGUUCUG | 1214 |
| R5249_CasPhi12_S | AUUGCUCCUUACGAGGAGACGCAGCCAACAGCACCUC | 1215 |
| R5250_CasPhil2_S | AUUGCUCCUUACGAGGAGACGCUGCCAAGGAGCACCG | 1216 |
| R5251_CasPhi12_S | AUUGCUCCUUACGAGGAGACCCCAGCACAGCAAUCAC | 1217 |
| R5252_CasPhi12_S | AUUGCUCCUUACGAGGAGACGCCCAGCACAGCAAUCA | 1218 |
| R5253_CasPhi12_S | AUUGCUCCUUACGAGGAGACCUGUGCUGGGCAAAGCU | 1219 |
| R5254_CasPhi12_S | AUUGCUCCUUACGAGGAGACCCCUGACCAGCUUUGCC | 1220 |
| R5255_CasPhi12_S | AUUGCUCCUUACGAGGAGACGGCUGGGGCAGUGAGCC | 1221 |
| R5256_CasPhi12_S | AUUGCUCCUUACGAGGAGACUGGCCGGCUUCCCCAGU | 1222 |
| R5257_CasPhi12_S | AUUGCUCCUUACGAGGAGACCCCAGUACGACUUUGUC | 1223 |
| R5258_CasPhi12_S | AUUGCUCCUUACGAGGAGACGUCUUCUCUGUCCCCUG | 1224 |
| R5259_CasPhi12_S | AUUGCUCCUUACGAGGAGACUCUUCUCUGUCCCCUGC | 1225 |
| R5260_CasPhi12_S | AUUGCUCCUUACGAGGAGACUCUGUCCCCUGCCAUUG | 1226 |
| R5261_CasPhi12_S | AUUGCUCCUUACGAGGAGACAAGCAAUGGCAGGGGAC | 1227 |
| R5262_CasPhi12_S | AUUGCUCCUUACGAGGAGACCUUGAACCGUCCGGGGG | 1228 |
| R5263_CasPhi12_S | AUUGCUCCUUACGAGGAGACAACCGUCCGGGGGAUGC | 1229 |
| R5264_CasPhi12_S | AUUGCUCCUUACGAGGAGACUCCCUGGGCCCACAGCC | 1230 |
| R5265_CasPhi12_S | AUUGCUCCUUACGAGGAGACAAGAUGUGGCUGAAAAC | 1231 |
| R5266_CasPhi12_S | AUUGCUCCUUACGAGGAGACUCAGCCACAUCUUGAAG | 1232 |
| R5267_CasPhi12_S | AUUGCUCCUUACGAGGAGACCAGCCACAUCUUGAAGA | 1233 |
| R5268_CasPhi12_S | AUUGCUCCUUACGAGGAGACAGCCACAUCUUGAAGAG | 1234 |
| R5269_CasPhi12_S | AUUGCUCCUUACGAGGAGACAAGAGACCUGACCGCGU | 1235 |
| R5270_CasPhi12_S | AUUGCUCCUUACGAGGAGACUGCUCAUCCUAGACGGC | 1236 |
| R5271_CasPhi12_S | AUUGCUCCUUACGAGGAGACCAGCUCCUCGAAGCCGU | 1237 |
| R5272_CasPhi12_S | AUUGCUCCUUACGAGGAGACCGCUUCCAGCUCCUCGA | 1238 |
| R5273_CasPhi12_S | AUUGCUCCUUACGAGGAGACGAGGAGCUGGAAGCGCA | 1239 |
| R5274_CasPhi12_S | AUUGCUCCUUACGAGGAGACCUGCACAGCACGUGCGG | 1240 |
| R5275_CasPhi12_S | AUUGCUCCUUACGAGGAGACUGGAAAAGGCCGGCCAG | 1241 |
| R5276_CasPhi12_S | AUUGCUCCUUACGAGGAGACUUCUGGAAAAGGCCGGC | 1242 |
| R5277_CasPhil2_S | AUUGCUCCUUACGAGGAGACUCCAGAAGAAGCUGCUC | 1243 |
| R5278_CasPhi12_S | AUUGCUCCUUACGAGGAGACCCAGAAGAAGCUGCUCC | 1244 |
| R5279_CasPhi12_S | AUUGCUCCUUACGAGGAGACCAGAAGAAGCUGCUCCG | 1245 |
| R5280_CasPhil2_S | AUUGCUCCUUACGAGGAGACCACCCUCCUCCUCACAG | 1246 |
| R5281_CasPhi12_S | AUUGCUCCUUACGAGGAGACCUCAGGCUCUGGACCAG | 1247 |
| R5282_CasPhi12_S | AUUGCUCCUUACGAGGAGACGAGCUGUCCGGCUUCUC | 1248 |
| R5283_CasPhi12_S | AUUGCUCCUUACGAGGAGACAGCUGUCCGGCUUCUCC | 1249 |
| R5284_CasPhi12_S | AUUGCUCCUUACGAGGAGACUCCAUGGAGCAGGCCCA | 1250 |
| R5285_CasPhi12_S | AUUGCUCCUUACGAGGAGACGAGAGCUCAGGGAUGAC | 1251 |
| R5286_CasPhi12_S | AUUGCUCCUUACGAGGAGACAGAGCUCAGGGAUGACA | 1252 |
| R5287_CasPhi12_S | AUUGCUCCUUACGAGGAGACGUGCUCUGUCAUCCCUG | 1253 |
| R5288_CasPhi12_S | AUUGCUCCUUACGAGGAGACUUCUCAGUCACAGCCAC | 1254 |
| R5289_CasPhi12_S | AUUGCUCCUUACGAGGAGACUCAGUCACAGCCACAGC | 1255 |
| R5290_CasPhi12_S | AUUGCUCCUUACGAGGAGACGUGCCGGGCAGUGUGCC | 1256 |
| R5291_CasPhi12_S | AUUGCUCCUUACGAGGAGACUGCCGGGCAGUGUGCCA | 1257 |
| R5292_CasPhi12_S | AUUGCUCCUUACGAGGAGACGCGUCCUCCCCAAGCUC | 1258 |
| R5293_CasPhi12_S | AUUGCUCCUUACGAGGAGACGGGAGGACGCCAAGCUG | 1259 |
| R5294_CasPhi12_S | AUUGCUCCUUACGAGGAGACGCCAGCUCUGCCAGGGC | 1260 |
| R5295_CasPhi12_S | AUUGCUCCUUACGAGGAGACAUGUCUGCGGCCCAGCU | 1261 |
| R5392_CasPhi12_S | AUUGCUCCUUACGAGGAGACGAUGUCUGCGGCCCAGC | 1262 |
| R5393_CasPhi12_S | AUUGCUCCUUACGAGGAGACCCAUCCGCAGACGUGAG | 1263 |
| R5394_CasPhi12_S | AUUGCUCCUUACGAGGAGACGCCAUCGCCCAGGUCCU | 1264 |
| R5395_CasPhi12_S | AUUGCUCCUUACGAGGAGACGGCCAUCGCCCAGGUCC | 1265 |
| R5396_CasPhi12_S | AUUGCUCCUUACGAGGAGACGACUAAGCCUUUGGCCA | 1266 |
| R5397_CasPhi12_S | AUUGCUCCUUACGAGGAGACGUCCAACACCCACCGCG | 1267 |
| R5398_CasPhi12_S | AUUGCUCCUUACGAGGAGACCAGGAGGAAGCUGGGGA | 1268 |
| R5399_CasPhil2_S | AUUGCUCCUUACGAGGAGACCCCAGCUUCCUCCUGCA | 1269 |
| R5400_CasPhi12_S | AUUGCUCCUUACGAGGAGACCUCCUGCAAUGCUUCCU | 1270 |
| R5401_CasPhi12_S | AUUGCUCCUUACGAGGAGACCUGGGGGCCCUGUGGCU | 1271 |
| R5402_CasPhil2_S | AUUGCUCCUUACGAGGAGACGCCACUCAGAGCCAGCC | 1272 |
| R5403_CasPhi12_S | AUUGCUCCUUACGAGGAGACCGCCACUCAGAGCCAGC | 1273 |
| R5404_CasPhi12_S | AUUGCUCCUUACGAGGAGACAUUUCGCCACUCAGAGC | 1274 |
| R5405_CasPhil2_S | AUUGCUCCUUACGAGGAGACUCCUUGAUUUCGCCACU | 1275 |
| R5406_CasPhi12_S | AUUGCUCCUUACGAGGAGACGGGUCAAUGCUAGGUAC | 1276 |
| R5407_CasPhi12_S | AUUGCUCCUUACGAGGAGACCUUGGGGUCAAUGCUAG | 1277 |
| R5408_CasPhi12_S | AUUGCUCCUUACGAGGAGACUUCCUUGGGGUCAAUGC | 1278 |
| R5409_CasPhi12_S | AUUGCUCCUUACGAGGAGACACCCCAAGGAAGAAGAG | 1279 |
| R5410_CasPhi12_S | AUUGCUCCUUACGAGGAGACUCAUAGGGCCUCUUCUU | 1280 |
| R5411_CasPhi12_S | AUUGCUCCUUACGAGGAGACCUGGCUGGGCUGAUCUU | 1281 |
| R5412_CasPhi12_S | AUUGCUCCUUACGAGGAGACUGGCUGGGCUGAUCUUC | 1282 |
| R5413_CasPhi12_S | AUUGCUCCUUACGAGGAGACCAGCCUCCCGCCCGCUG | 1283 |
| R5414_CasPhi12_S | AUUGCUCCUUACGAGGAGACCUGUCCACCGAGGCAGC | 1284 |
| R5415_CasPhi12_S | AUUGCUCCUUACGAGGAGACUGCUUCCUGUCCACCGA | 1285 |
| R5416_CasPhi12_S | AUUGCUCCUUACGAGGAGACAGGUACCUCGCAAGCAC | 1286 |
| R5417_CasPhi12_S | AUUGCUCCUUACGAGGAGACCGAGGUACCUGAAGCGG | 1287 |
| R5418_CasPhi12_S | AUUGCUCCUUACGAGGAGACCAGCCUCCUCGGCCUCG | 1288 |
| R5419_CasPhi12_S | AUUGCUCCUUACGAGGAGACGGCAGCACGUGGUACAG | 1289 |
| R5420_CasPhi12_S | AUUGCUCCUUACGAGGAGACGCAGCACGUGGUACAGG | 1290 |
| R5421_CasPhi12_S | AUUGCUCCUUACGAGGAGACUCUGGGCACCCGCCUCA | 1291 |
| R5422_CasPhi12_S | AUUGCUCCUUACGAGGAGACCUGGGCACCCGCCUCAC | 1292 |
| R5423_CasPhi12_S | AUUGCUCCUUACGAGGAGACUGGGCACCCGCCUCACG | 1293 |
| R5424_CasPhi12_S | AUUGCUCCUUACGAGGAGACCCCAGUACAUGUGCAUC | 1294 |
| R5425_CasPhi12_S | AUUGCUCCUUACGAGGAGACGCCCGCCGCCUCCAAGG | 1295 |
| R5426_CasPhi12_S | AUUGCUCCUUACGAGGAGACGAGGCGGCGGGCCAAGA | 1296 |
| R5427_CasPhi12_S | AUUGCUCCUUACGAGGAGACUCCCUGGACCUCCGCAG | 1297 |
| R5428_CasPhi12_S | AUUGCUCCUUACGAGGAGACGCCCCUCUGGAUUGGGG | 1298 |
| R5429_CasPhil2_S | AUUGCUCCUUACGAGGAGACCCCCUCUGGAUUGGGGA | 1299 |
| R5430_CasPhi12_S | AUUGCUCCUUACGAGGAGACGGGAGCCUCGUGGGACU | 1300 |
| R5431_CasPhi12_S | AUUGCUCCUUACGAGGAGACGUCUCCCCAUGCUGCUG | 1301 |
| R5432_CasPhi12_S | AUUGCUCCUUACGAGGAGACUCCUCUGCUGCCUGAAG | 1302 |
| R5433_CasPhi12_S | AUUGCUCCUUACGAGGAGACAGGCAGCAGAGGAGAAG | 1303 |
| R5434_CasPhi12_S | AUUGCUCCUUACGAGGAGACAAAGGCUCGAUGGUGAA | 1304 |
| R5435_CasPhi12_S | AUUGCUCCUUACGAGGAGACGAAAGGCUCGAUGGUGA | 1305 |
| R5436_CasPhi12_S | AUUGCUCCUUACGAGGAGACACCAUCGAGCCUUUCAA | 1306 |
| R5437_CasPhi12_S | AUUGCUCCUUACGAGGAGACGCUUUGAAAGGCUCGAU | 1307 |
| R5438_CasPhi12_S | AUUGCUCCUUACGAGGAGACAGGGACUUGGCUUUGAA | 1308 |
| R5439_CasPhi12_S | AUUGCUCCUUACGAGGAGACCAAAGCCAAGUCCCUGA | 1309 |
| R5440_CasPhi12_S | AUUGCUCCUUACGAGGAGACAAAGCCAAGUCCCUGAA | 1310 |
| R5441_CasPhi12_S | AUUGCUCCUUACGAGGAGACCACAUCCUUCAGGGACU | 1311 |
| R5442_CasPhi12_S | AUUGCUCCUUACGAGGAGACCCAGGUCUUCCACAUCC | 1312 |
| R5443_CasPhi12_S | AUUGCUCCUUACGAGGAGACCCCAGGUCUUCCACAUC | 1313 |
| R5444_CasPhi12_S | AUUGCUCCUUACGAGGAGACCUCGGAAGACACAGCUG | 1314 |
| R5445_CasPhi12_S | AUUGCUCCUUACGAGGAGACGGUCCCGAACAGCAGGG | 1315 |
| R5446_CasPhi12_S | AUUGCUCCUUACGAGGAGACAGGUCCCGAACAGCAGG | 1316 |
| R5447_CasPhi12_S | AUUGCUCCUUACGAGGAGACUUUAGGUCCCGAACAGC | 1317 |
| R5448_CasPhi12_S | AUUGCUCCUUACGAGGAGACCUUUAGGUCCCGAACAG | 1318 |
| R5449_CasPhi12_S | AUUGCUCCUUACGAGGAGACGGGACCUAAAGAAACUG | 1319 |
| R5450_CasPhi12_S | AUUGCUCCUUACGAGGAGACGGGAAAGCCUGGGGGCC | 1320 |
| R5451_CasPhi12_S | AUUGCUCCUUACGAGGAGACGGGGAAAGCCUGGGGGC | 1321 |
| R5452_CasPhi12_S | AUUGCUCCUUACGAGGAGACCCCCAAACUGGUGCGGA | 1322 |
| R5453_CasPhi12_S | AUUGCUCCUUACGAGGAGACCCCAAACUGGUGCGGAU | 1323 |
| R5454_CasPhil2_S | AUUGCUCCUUACGAGGAGACUUCUCACUCAGCGCAUC | 1324 |
| R5455_CasPhi12_S | AUUGCUCCUUACGAGGAGACAGCUGGGGGAAGGUGGC | 1325 |
| R5456_CasPhi12_S | AUUGCUCCUUACGAGGAGACCCCCAGCUGAAGUCCUU | 1326 |
| R5457_CasPhil2_S | AUUGCUCCUUACGAGGAGACCAAGGACUUCAGCUGGG | 1327 |
| R5458_CasPhi12_S | AUUGCUCCUUACGAGGAGACCCAAGGACUUCAGCUGG | 1328 |
| R5459_CasPhil2_S | AUUGCUCCUUACGAGGAGACAGGGUUUCCAAGGACUU | 1329 |
| R5460_CasPhi12_S | AUUGCUCCUUACGAGGAGACUAGGCACCCAGGUCAGU | 1330 |
| R5461_CasPhil2_S | AUUGCUCCUUACGAGGAGACGUAGGCACCCAGGUCAG | 1331 |
| R5462_CasPhi12_S | AUUGCUCCUUACGAGGAGACGCUCGCUGCAUCCCUGC | 1332 |
| R5463_CasPhi12_S | AUUGCUCCUUACGAGGAGACGCCUGAGCAGGGAUGCA | 1333 |
| R5464_CasPhil2_S | AUUGCUCCUUACGAGGAGACUACAAUAACUGCAUCUG | 1334 |
| R5465_CasPhi12_S | AUUGCUCCUUACGAGGAGACGCUCGUGUGCUUCCGGA | 1335 |
| R5466_CasPhil2_S | AUUGCUCCUUACGAGGAGACCGGACAUGGUGUCCCUC | 1336 |
| R5467_CasPhil2_S | AUUGCUCCUUACGAGGAGACACGGCUGCCGGGGCCCA | 1337 |
| R5468_CasPhi12_S | AUUGCUCCUUACGAGGAGACGGAGGUGUCCUCAUGUG | 1338 |
| R5469_CasPhil2_S | AUUGCUCCUUACGAGGAGACCUGGACACUGAAUGGGA | 1339 |
| R5470_CasPhi12_S | AUUGCUCCUUACGAGGAGACAGUGUCCAGGAACACCU | 1340 |
| R5471_CasPhil2_S | AUUGCUCCUUACGAGGAGACCAGGUGUUCCUGGACAC | 1341 |
| R5472_CasPhil2_S | AUUGCUCCUUACGAGGAGACUUGCAGGUGUUCCUGGA | 1342 |
| R5473_CasPhi12_S | AUUGCUCCUUACGAGGAGACACGGAUCAGCCUGAGAU | 1343 |

### EXAMPLES

### Example 1. AAV Vector Encoding CasΦ.12 and Guide RNAs Edit PCSK9 in Mammalian Cells

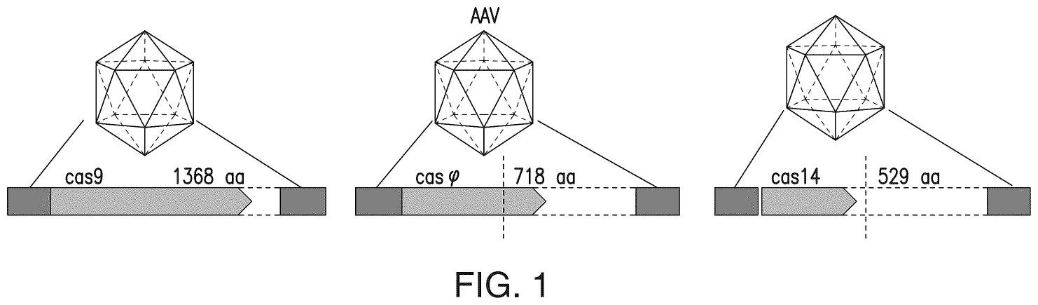

This example demonstrates that genome editing can be performed with an AAV vector encoding a Cas effector protein having a length of between 700 and 800 amino acids as depicted in FIG. 1 (CasΦ.12) and a guide RNA targeting PCSK9. Several guide RNAs with varying repeat lengths (nucleotide sequence that is capable of being non-covalently bound by an effector protein) of 36, 25, 20, or 19 nucleotides in combination with spacer lengths (nucleotide sequence that hybridizes to a target nucleic acid) of 20, 17, or 16 nucleotides were tested. Each guide RNA was cloned into an AAV vector with a U6 promoter to drive guide RNA expression, and an intron-less EF1alpha short (EFS) promoter driving CasΦ.12 expression. The AAV vector also included a polyA signal and 1 kb stuffer sequence. Hepal-6 mouse hepatoma cells were nucleofected with 10 μg of AAV plasmid. After 72 hours, genomic DNA was extracted and the frequency of indel mutations was determined using NGS.

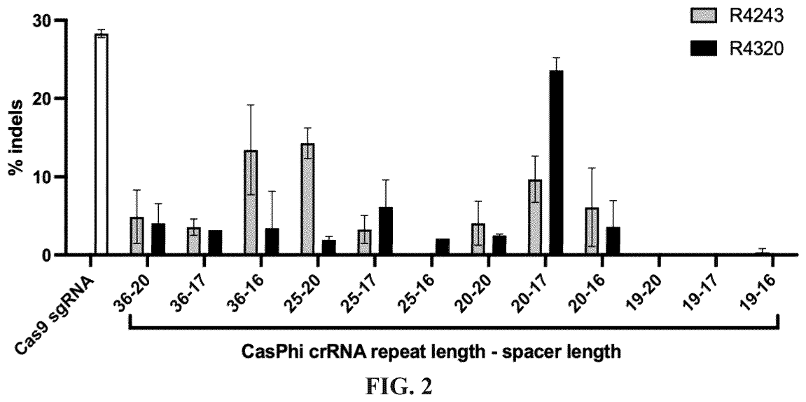

FIG. 2 shows the frequency of CasΦ.12 induced indel mutations in Hepal-6 cells transduced with 10 μg of each AAV plasmid. In the graph legend, repeat and spacer lengths are indicated as the number of nucleotides in the repeat followed by the number of nucleotides in the spacer, e.g., 20-17 has a repeat length of 20 nucleotides and a spacer length of 17 nucleotides. The frequency of indel mutations is comparable to that of Cas9. This study demonstrates that a vector encoding a guide RNA and CasΦ.12 provide robust genome editing across different gRNA sequences and with gRNAs of different repeat and spacer lengths.

### Example 2: CasM.19952 edits genomic DNA in mammalian cells

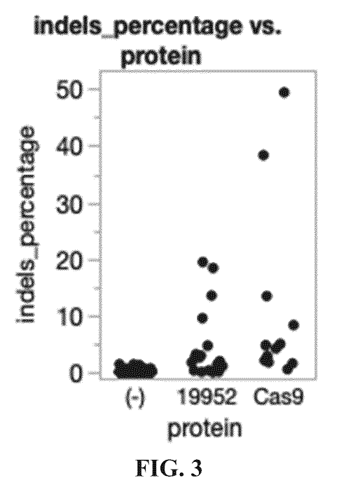

CasM.19952 was tested for its ability to produce indels in HEK293T cells. Briefly, a plasmid encoding CasM.19952 and a guide RNA was delivered by lipofection to HEK293T cells. This was performed for a variety of guide RNAs targeting up to twenty-four loci adjacent to biochemically determined PAM sequences. Indels were detected by next generation sequencing of PCR amplicons at the targeted loci and indel percentage was calculated as the fraction of sequencing reads containing insertions or deletions relative to an unedited reference sequence. Sequencing libraries with less than 2000 of reads aligning to the reference sequence were excluded from the analysis for quality control purposes. “No plasmid” and SpyCas9 were included as negative and positive controls, respectively. FIG. 3 shows the results. TABLE 17 describes the sequences of the single guide RNAs tested that provided the greatest percent of reads with indels. Non-bold, non-italicized, capital letters indicate the tracrRNA sequence region of the guide RNA; italicized letters indicate a linker; bold letters indicate the repeat sequence; and the lowercase letters represent the spacer sequence. This experiment demonstrated that CasM.19952 is a robust editor of genomic DNA in mammalian cells.

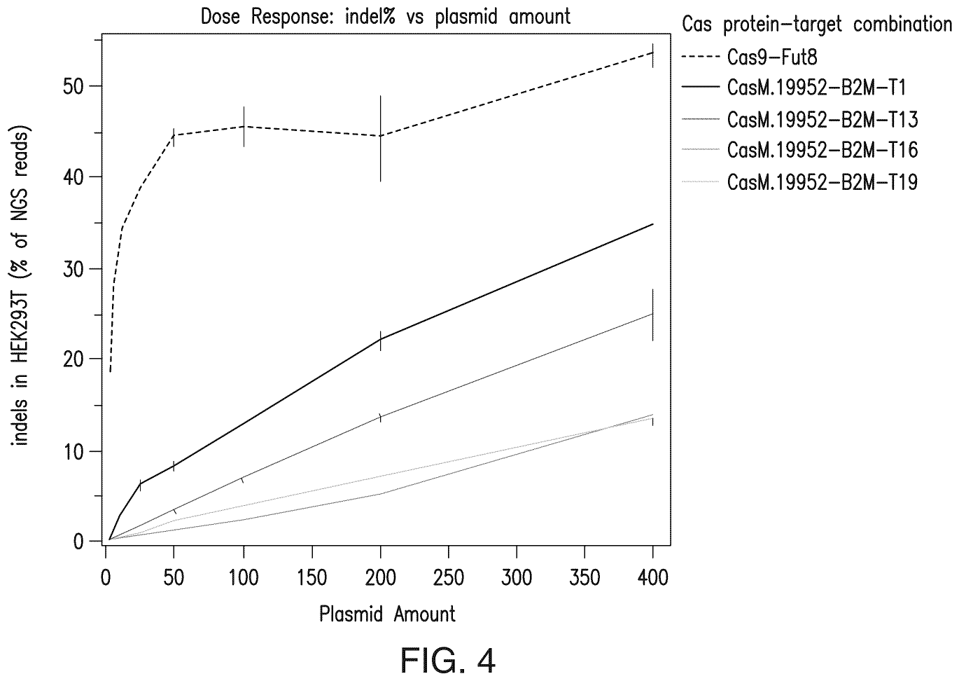

A dose-response experiment confirmed the genome editing capability of CasM.19952 in mammalian cells. Plasmids encoding CasM.19952 and single guide RNAs were delivered at various concentrations by lipofection into HEK293T. CasM.19952 was programmed to target four loci. SpyCas9 was included as a positive control. Indels were observed at all four loci. Results are shown in FIG. 4.

TABLE 17
sgRNAs that provided genome editing with CasM.19952 in HEK293T cells

| | | % of |
|---|---|---|
| | | reads |
| sgRNA Sequence | | with |
| DNA Sequence | RNA Sequence | indels |
| TGGGGCAGTTGGTTGCCCTTAGCC | UGGGGCAGUUGGUUGCCCUUAGC | 13.47 |
| TGAGGCATTTATTGCACTCGGGAA | CUGAGGCAUUUAUUGCACUCGGG | |
| GTACCATTTCTCAGAAATGGTACA | AAGUACCAUUUCUCAGAAAUGGU | |
| TCCAACtctaggegcccgctaagttc (SEQ ID | ACAUCCAACucuaggcgcccgcuaaguuc | |
| NO: 1344) | (SEQ ID NO: 2429) | |
| TGGGGCAGTTGGTTGCCCTTAGCC | UGGGGCAGUUGGUUGCCCUUAGC | 4.63 |
| TGAGGCATTTATTGCACTCGGGAA | CUGAGGCAUUUAUUGCACUCGGG | |
| GTACCATTTCTCAGAAATGGTACA | AAGUACCAUUUCUCAGAAAUGGU | |
| TCCAACcccgggtaagcctgtctgct (SEQ ID | ACAUCCAACcccggguaagccugucugcu | |
| NO: 1345) | (SEQ ID NO: 2430) | |
| TGGGGCAGTTGGTTGCCCTTAGCC | UGGGGCAGUUGGUUGCCCUUAGC | 19.40 |
| TGAGGCATTTATTGCACTCGGGAA | CUGAGGCAUUUAUUGCACUCGGG | |
| GTACCATTTCTCAGAAATGGTACA | AAGUACCAUUUCUCAGAAAUGGU | |
| TCCAACcgtgctgtttcctccccacg (SEQ ID | ACAUCCAACcgugcuguuuccuccccacg | |
| NO: 1346) | (SEQ ID NO: 2431) | |
| TGGGGCAGTTGGTTGCCCTTAGCC | UGGGGCAGUUGGUUGCCCUUAGC | 3.15 |
| TGAGGCATTTATTGCACTCGGGAA | CUGAGGCAUUUAUUGCACUCGGG | |
| GTACCATTTCTCAGAAATGGTACA | AAGUACCAUUUCUCAGAAAUGGU | |
| TCCAACgtgccttagtttcttcatct (SEQ ID | ACAUCCAAgugccuuaguuuuucaucu | |
| NO: 1347) | (SEQ ID NO: 2432) | |
| TGGGGCAGTTGGTTGCCCTTAGCC | UGGGGCAGUUGGUUGCCCUUAGC | 18.35 |
| TGAGGCATTTATTGCACTCGGGAA | CUGAGGCAUUUAUUGCACUCGGG | |
| GTACCATTTCTCAGAAATGGTACA | AAGUACCAUUUCUCAGAAAUGGU | |
| TCCAACgggggcgggggggagaaaaa (SEQ | ACAUCCAACggggggggggggagaaaaa | |
| ID NO: 1348) | (SEQ ID NO: 2433) | |
| TGGGGCAGTTGGTTGCCCTTAGCC | UGGGGCAGUUGGUUGCCCUUAGC | 9.48 |
| TGAGGCATTTATTGCACTCGGGAA | CUGAGGCAUUUAUUGCACUCGGG | |
| GTACCATTTCTCAGAAATGGTACA | AAGUACCAUUUCUCAGAAAUGGU | |
| TCCAACgcgccctccgatctggggtg (SEQ | ACAUCCAACgcgcccuccgaucuggggug | |
| ID NO: 1349) | (SEQ ID NO: 2434) | |

### Example 3. PAM Requirement for CasΦ Determined by In Vitro Enrichment

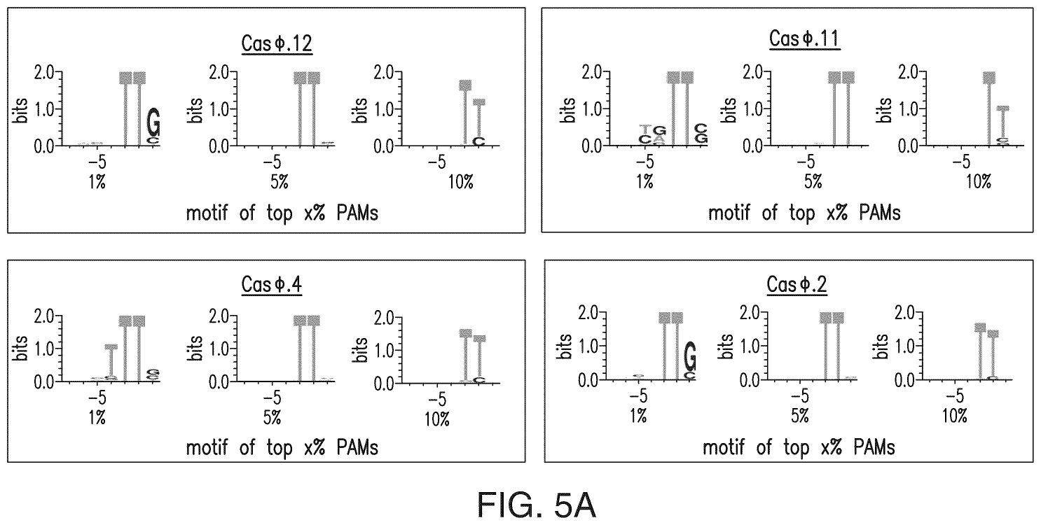

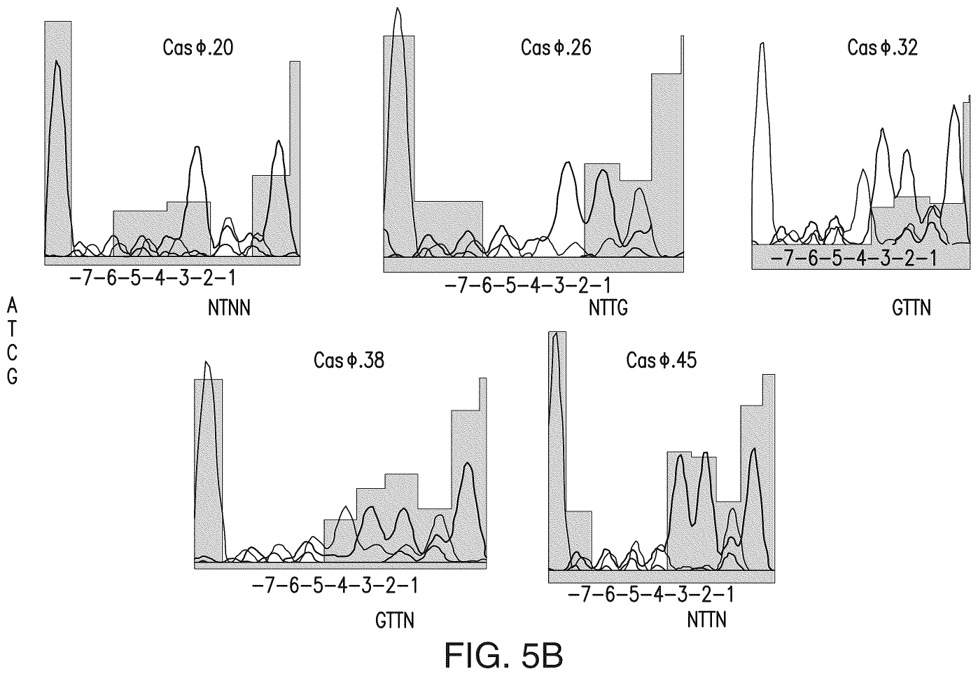

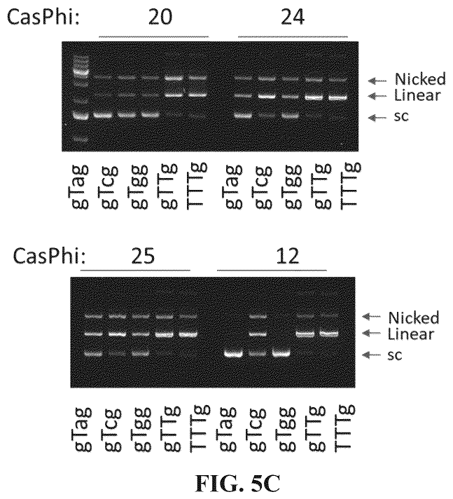

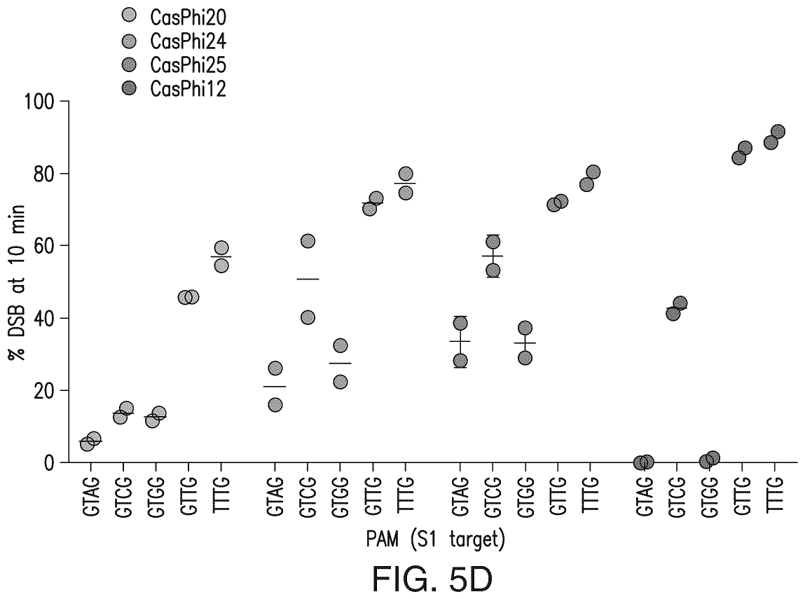

This example illustrates the NTTN PAM requirement for CasΦ.2, CasΦ.4, CasΦ.11 and CasΦ.12. An in vitro enrichment (IVE) analysis was performed. The CasΦ polypeptides were complexed with crRNA to form 500 nM RNP complexes at room temperature in NEB CutSmart buffer (50 mM Potassium Acetate, 20 mM Tris-Acetate, 10 mM Magnesium Acetate, 100 μg/ml BSA, pH 7.9 at 25° C.) for 30 minutes in a volume of 25 l. crRNA sequences are provided in TABLE 2. The cleavage incubation was performed at 37° C. and the reaction was quenched after 30 minutes. The substrate for the cleavage incubation was a pooled plasmid library which includes different PAM sequences. After quenching, the cleavage reactions were cleaned using Beckman SPRi beads. The samples were sequenced to identify which PAM sequences enabled target cleavage by the CasΦ polypeptides. As shown in FIG. 5A, this analysis revealed an NTTN PAM requirement for CasΦ.2, CasΦ.4, CasΦ.11 and CasΦ.12.

The inventors went on to assess the PAM requirement of CasΦ.20, CasΦ.26, CasΦ.32, CasΦ.38 and CasΦ.45. An IVE analysis was performed using the protocol described above for CasΦ.2, CasΦ.4, CasΦ.11 and CasΦ.12. As shown in FIG. 5B, Sanger sequencing revealed a NTNN PAM requirement for CasΦ.20, a NTTG PAM requirement for CasΦ.26, a GTTN PAM requirement for CasΦ.32 and CasΦ.38, and a NTTN PAM requirement for CasΦ.45.

The inventors also determined a single-base PAM requirement for CasΦ.20, CasΦ.24 and CasΦ.25. Amino acid sequences of the proteins used are shown in TABLE 1. The CasΦ polypeptides were complexed with their native crRNAs to form RNP complexes at room temperature for 20 minutes. crRNA sequences are provided in TABLE 2. The RNP complexes were incubated with target DNA at 37° C. for 60 minutes in NEB CutSmart buffer (50 mM Potassium Acetate, 20 mM Tris-Acetate, 10 mM Magnesium Acetate, 100 μg/ml BSA, pH 7.9 at 25° C.). The RNPs were then used in cleavage reactions with plasmid DNA comprising a target sequence and a PAM. Stating with a TTTg PAM, the PAM was mutated to each of the sequences shown in FIG. 5C to assess the PAM requirement. The products of the cleavage reactions were analyzed by gel electrophoresis, as seen in FIG. 5C. FIG. 5D provides the quantification of the gels shown in FIG. 5C. Together, the data in FIG. 5C and FIG. 5D demonstrate a NTNN PAM for DNA cleavage by CasΦ.20, CasΦ.24 and CasΦ.25.

This example demonstrates PAM sequences that enable CasΦ polypeptides to be targeted to a target sequence.

### Example 4. CasΦ-mediated genome editing in primary cells

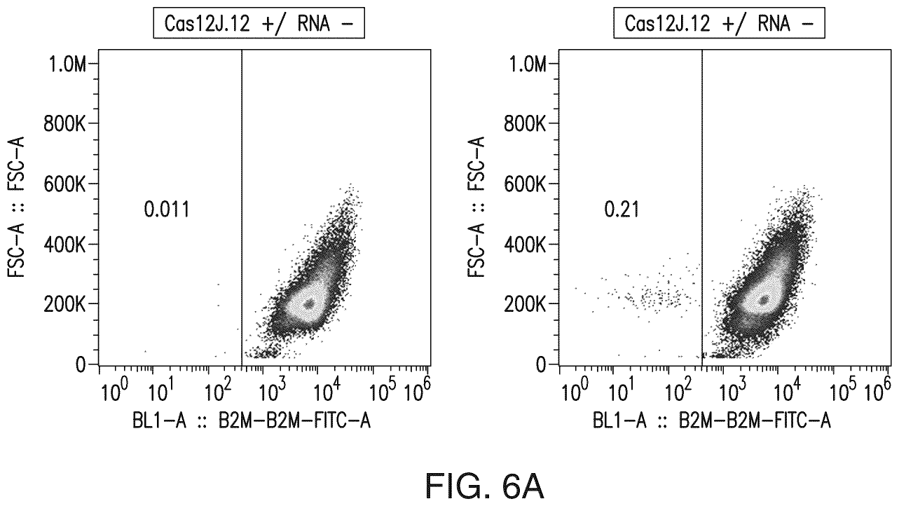

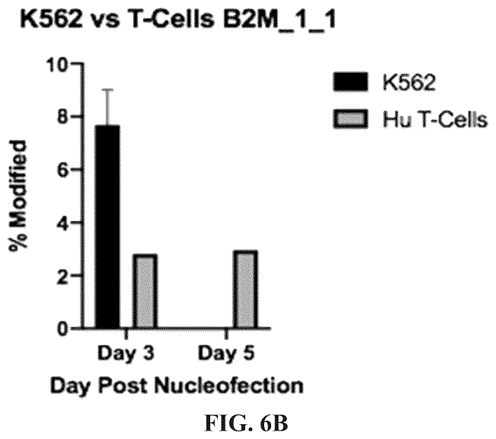

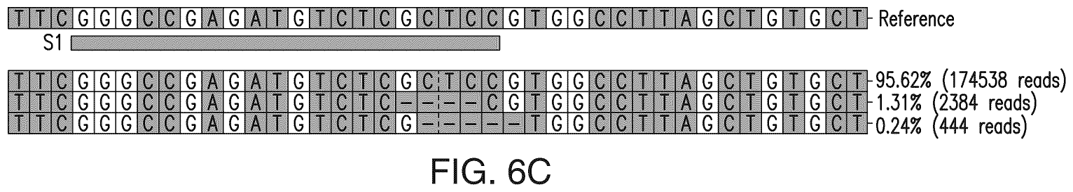

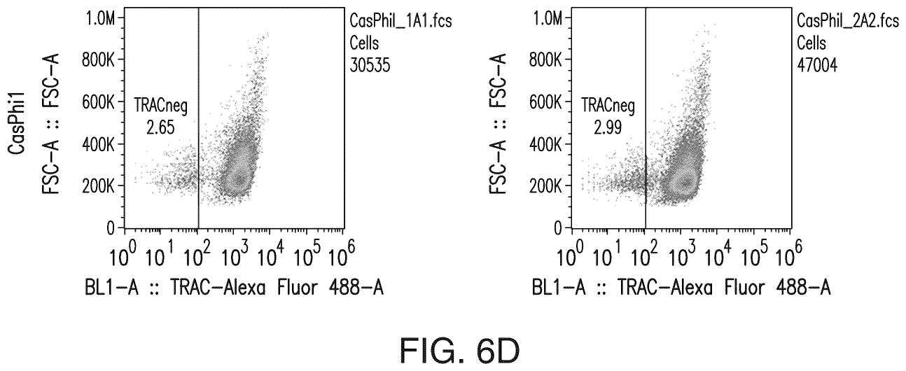

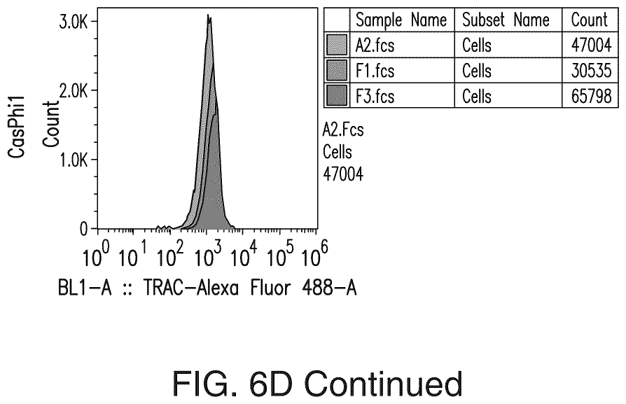

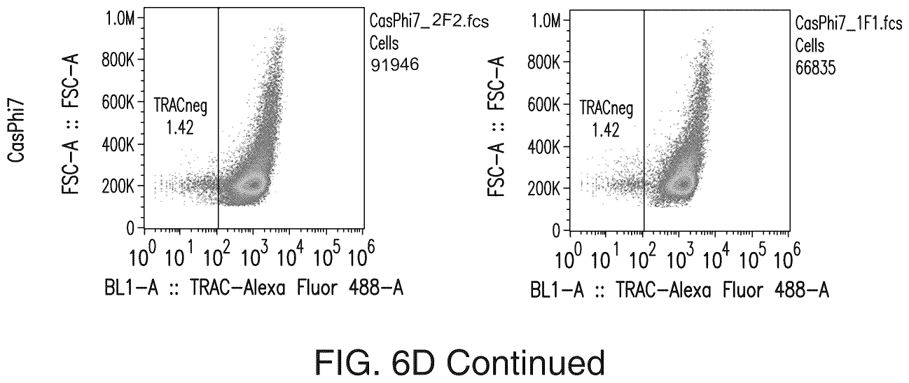

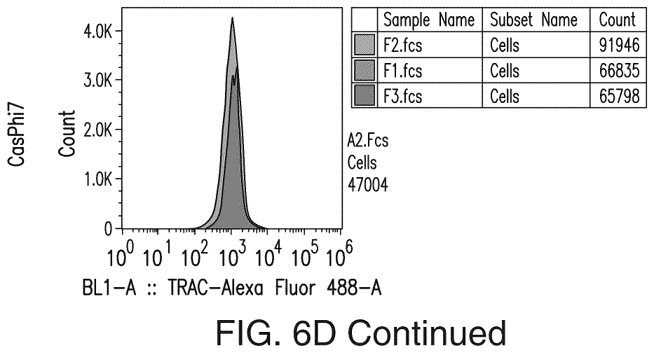

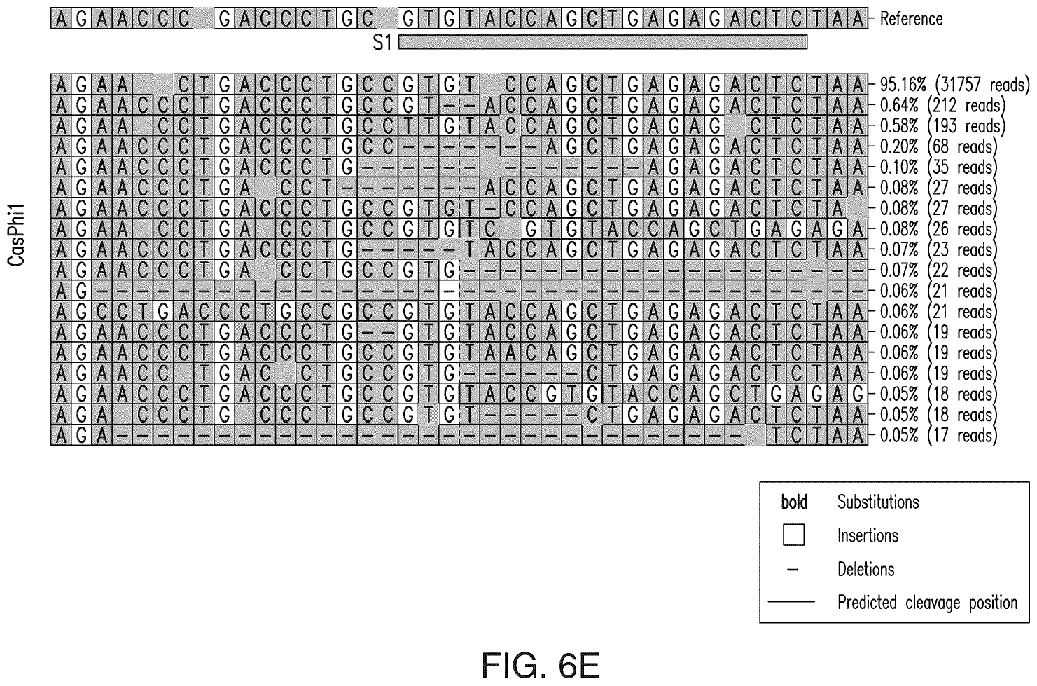

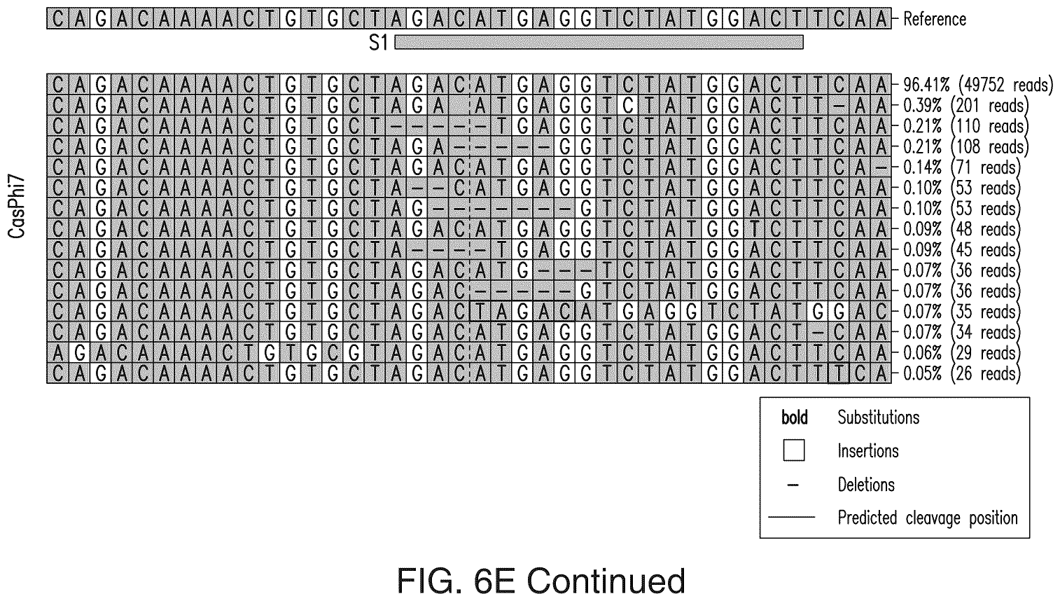

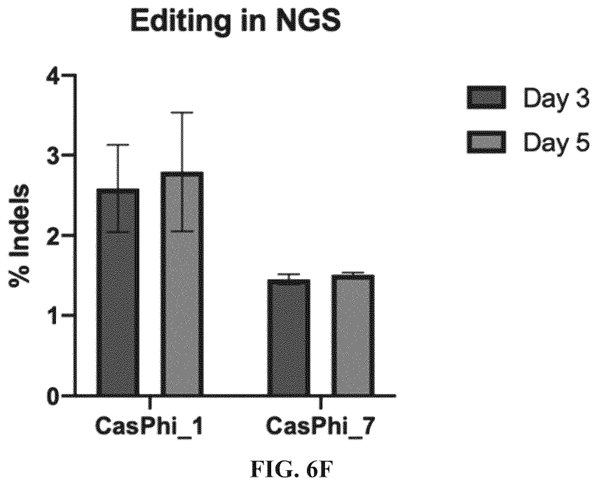

This example illustrates the ability of CasΦ polypeptides to mediate genome editing in primary cells, such as T cells. In this study, CasΦ.12 was delivered to human T cells. CasΦ.12 was complexed to its native crRNA comprising the spacer sequence 5′-GGGCCGAGAUGUCUCGCUCC-3′ (SEQ ID NO: 1368). Complexes were formed in a 3:1 ratio of crRNA:protein. For nucleofection, 50 pmol RNP was mixed with 320,000 cells per well and the Amaxa EH115 program was used. Immediately after nucleofection, 80 μl pre-warmed culture medium was added to each well. The cells were then left in the cuvette plate for 15 minutes before transfer to the culture plate. Genomic DNA was extracted from cells on day 3 and day 5. Flow cytometry analysis was performed on day 5. As shown in FIG. 6A, when CasΦ.12 was delivered with a gRNA targeting the endogenous beta-2 microglobulin (B2M) gene, a distinct population of B2M-negative cells was detected by flow cytometry analysis demonstrating the CasΦ.12-mediated knockout of the endogenous B2M gene. In the absence of the B2M-targeting gRNA, the population of B2M-negative cells was not observed by flow cytometry. Indels were confirmed by next generation sequencing analysis, as shown FIG. 6C, and quantified, as shown in FIG. 6B.

The inventors went on to use CasΦ.12 to target the T-cell receptor alpha-constant (TRAC) gene. Knockout of the TRAC gene prevents expression of the T cell receptor. Accordingly, TRAC knockout T cells are beneficial for T cell therapies (e.g., CAR-T cell therapies) because TRAC knockout T cells have a longer half-life in vivo as the T cells have less potential to attack the recipient's normal cells. In this study, CasΦ.12 and gRNA targeting the TRAC gene (CasPhi1 or CasPhi7) were delivered to T cells. As shown in FIG. 6D, the delivery of the CasΦ.12 and the gRNA resulted in a population of TRAC-negative cells, which were detected by flow cytometry. The inventors went on to confirm the presence of indel mutations by sequencing the target locus. As shown in FIG. 6E, the sequence analysis revealed insertion, deletion and substitution mutations at the endogenous targeted locus. The frequency of indel mutations was quantified, as shown in FIG. 6F.

These data demonstrate the utility of CasΦ polypeptides as a robust genome editing tool in primary human cells.

### Example 5. High Efficiency of CasΦ Polypeptide-Mediated Genome Editing in Primary Cells

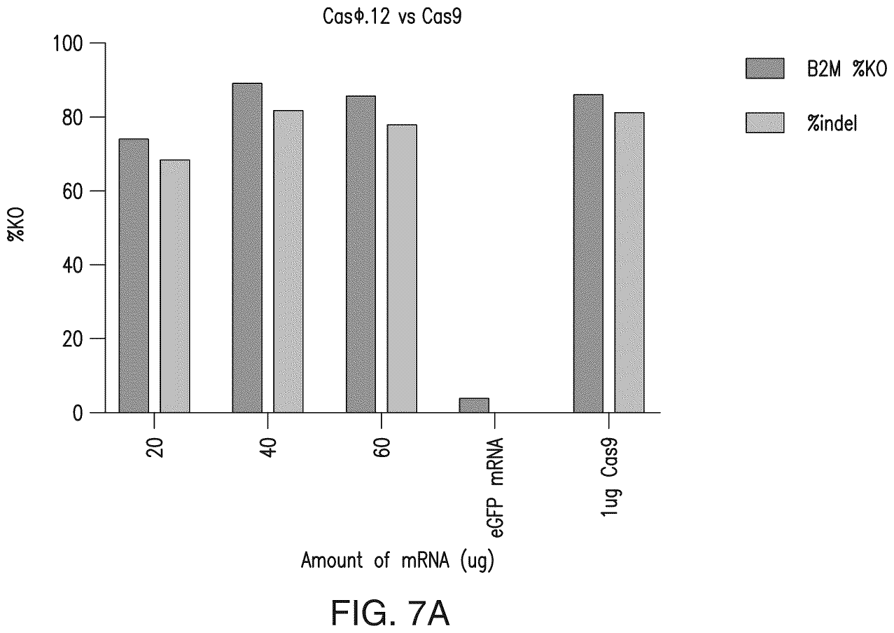

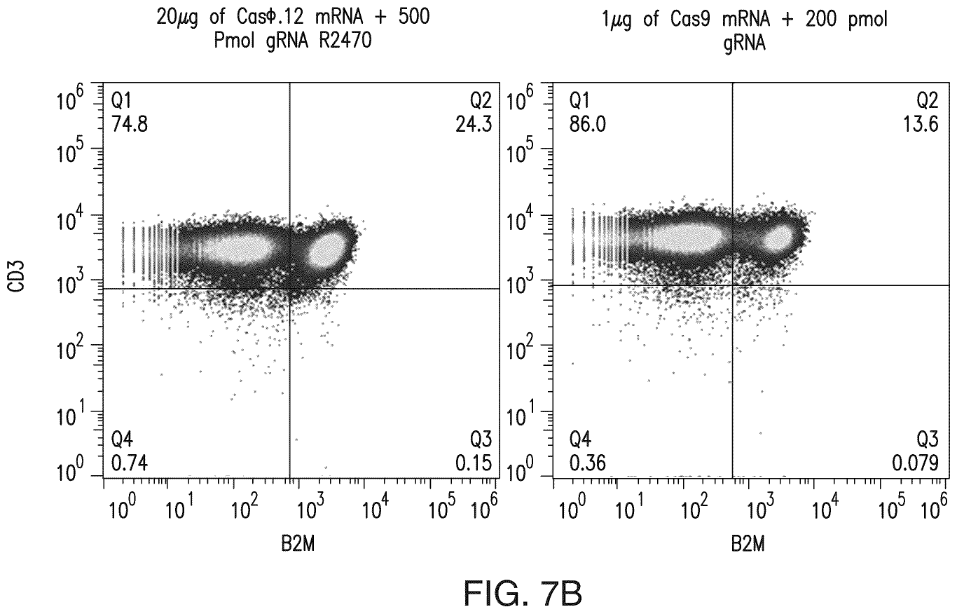

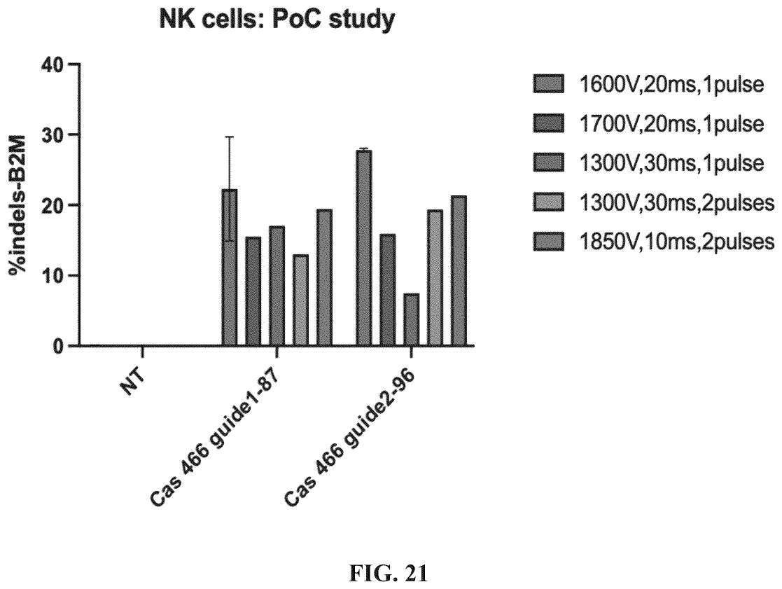

The present example shows that CasΦ.12 mediates high genome editing efficiency that is comparable the editing efficiency mediated by Cas9. Results of the study are shown in FIG. 21. In this study, CasΦ.12 mRNA (SEQ ID NO: 57) with a gRNA (CUUUCAAGACUAAUAGAUUGCUCCUUACGAGGAGACGGGCCGAGAUGUCUCGCUC C (SEQ ID NO: 1582)); spacer sequence is bold and underlined) or Cas9 mRNA with a gRNA (GGCCGAGATGTCTCGCTCCG (SEQ ID NO: 1583)) was delivered to T cells. gRNAs used in this study targeted the B2M gene. For nucleofection, T cells were resuspended in BTXpress electroporation medium (5×10⁵ cells per well) and mixed with CasΦ.12 or Cas9 mRNA and 500 pmol gRNA. Cells were collected on day 2 for extraction of genomic DNA, and the frequency of indel mutations was determined. As shown in FIG. 7A, when 20 μg of CasΦ.12 mRNA was delivered with gRNA to T cells, high genome editing efficiency was achieved, and this was at a similar level to of genome editing achieved using Cas9. Cells were also collected on Day 2 for flow cytometry to determine the frequency of B2M knockout. As shown in FIG. 7B and quantified in FIG. 7A, a similar percentage of B2M-negative cells were detected after delivery of CasΦ.12 or Cas9 mRNA. Accordingly, this example demonstrates high efficiency of CasΦ polypeptide-mediated genome efficiency in primary cells.

### Example 6

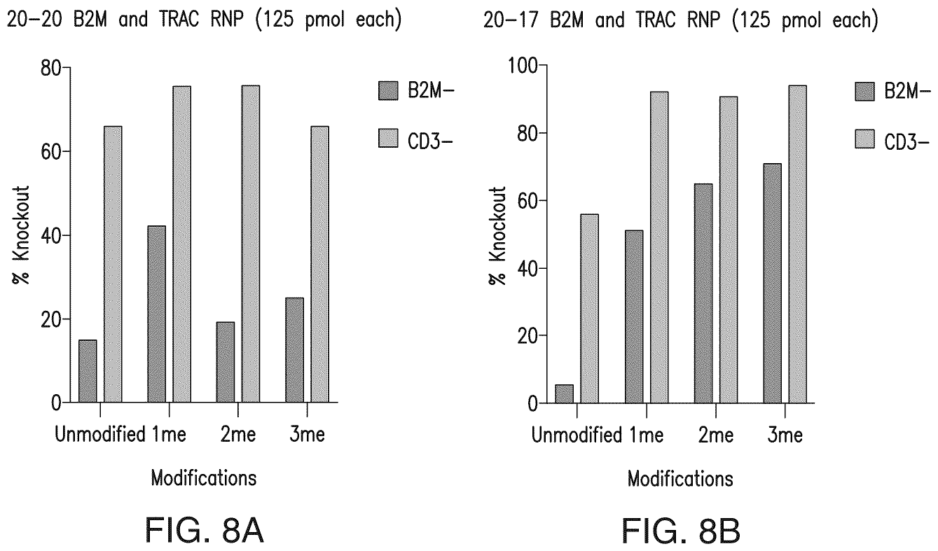

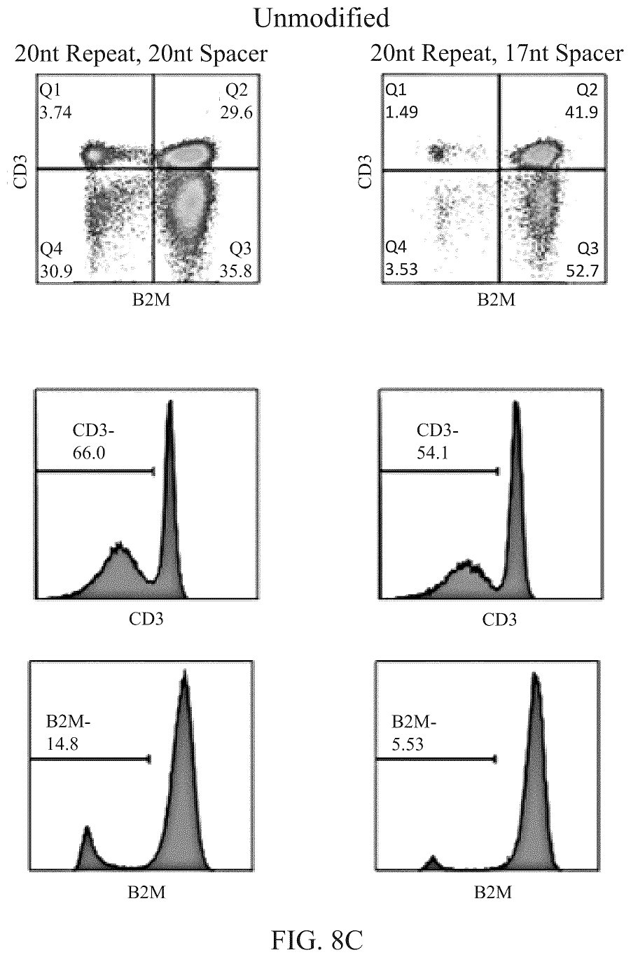

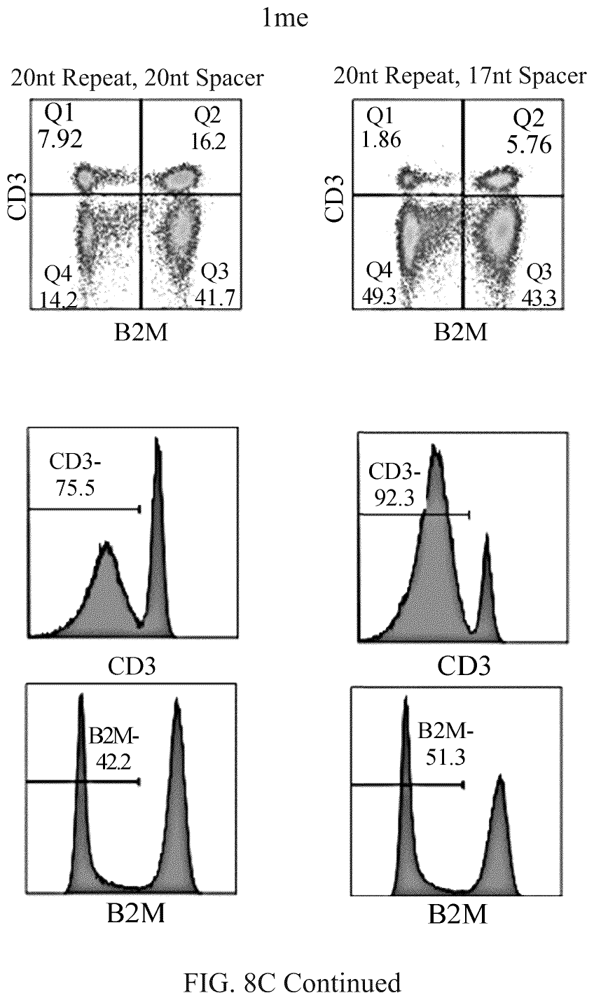

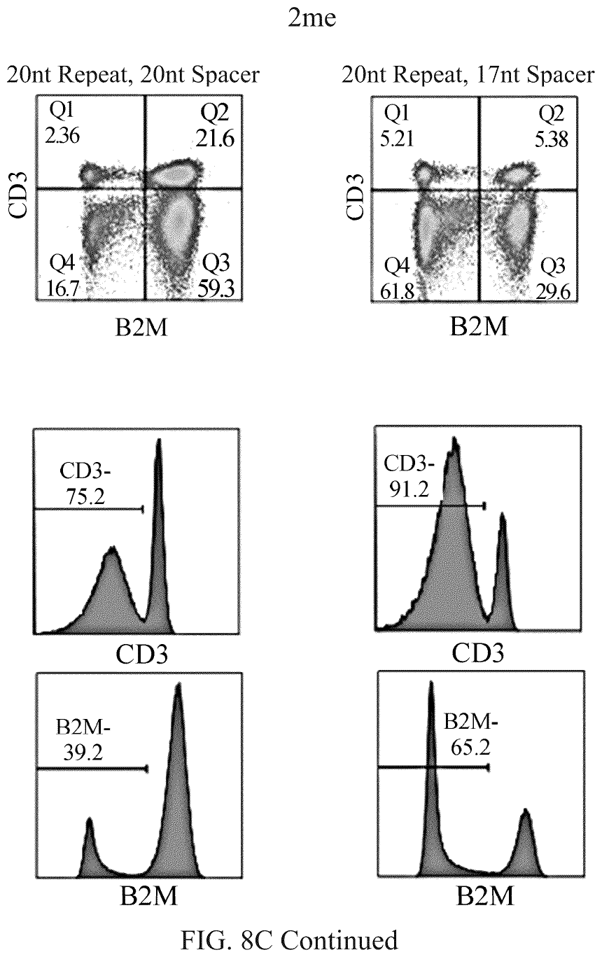

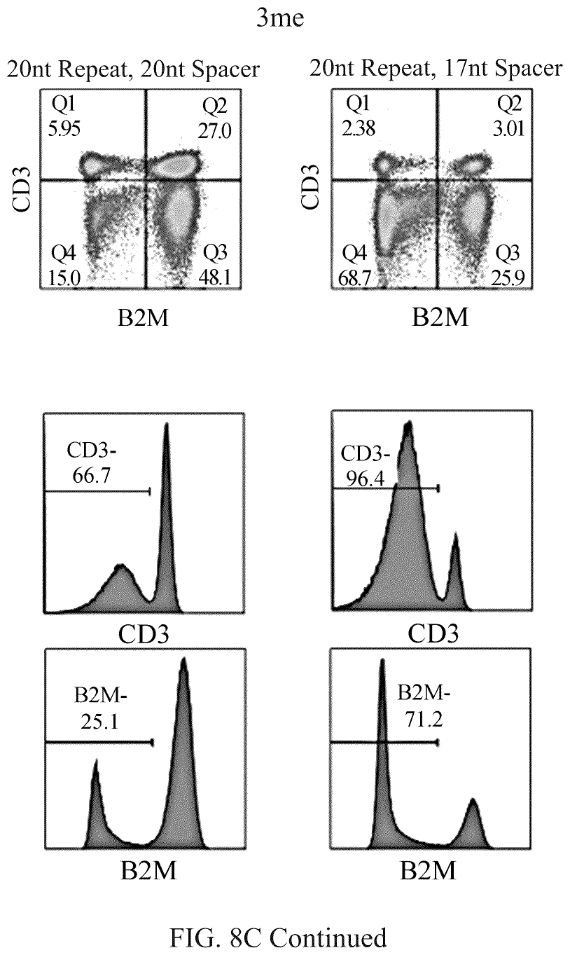

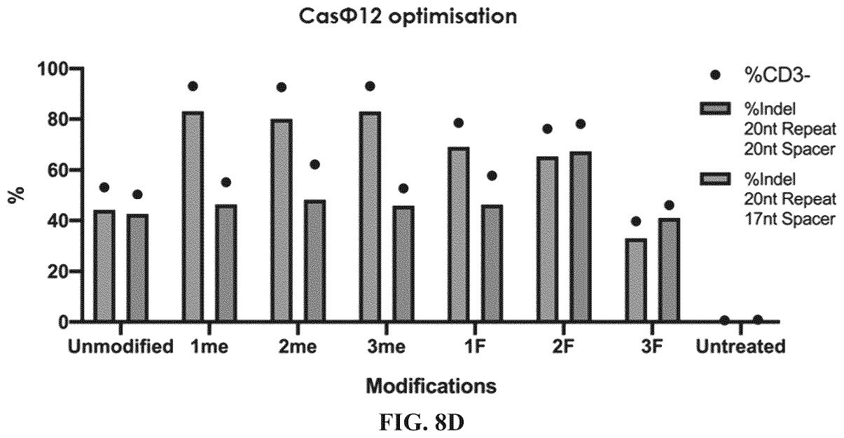

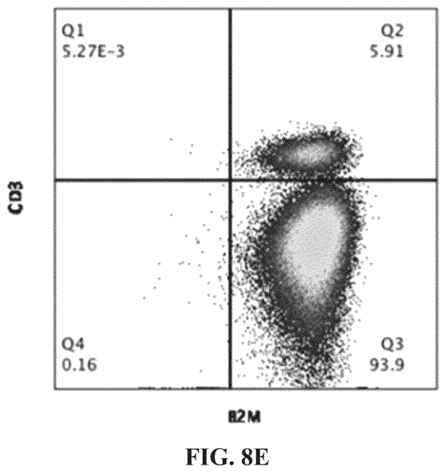

This example illustrates the ability of CasΦ RNP complexes to target multiple genes simultaneously. In this study, gRNAs targeting B2M or TRAC were incubated with CasΦ.12 polypeptides (SEQ ID NO: 57) for 10 minutes at room temperature to form RNP complexes. RNP complexes were formed with a variety of gRNAs with different modifications (unmodified, 2′-O-methyl on the last 3′ nucleotide of the crRNA (line), 2′-O-methyl on the last two 3′ nucleotides of the crRNA (2me) and 2′-O-methyl on the last three 3′ nucleotides of the crRNA(3me)) and with different repeat and spacer sequences (20-20, which corresponds to 20 nucleotide repeat and 20 nucleotide spacer, and 20-17, which corresponds to 20 nucleotide repeat and 17 nucleotide spacer), as shown in TABLE 18. B2M targeting RNPs, TRAC targeting RNPs or B2M targeting RNPs and TRAC targeting RNPs were added to T cells. T cells were resuspended at 5×10⁵ cells/20 μL in Nucleofection P3 solution and an Amaxa 4D 96-well electroporation system with pulse code EHi115 was used to nucleofect the cells. Immediately after nucleofection, 85 l pre-warmed culture medium was added to each well. The cells were then left in the cuvette plate for 10 minutes before transfer to the culture plate. On Day 3, genomic DNA was extracted. On Day 5, cells were harvested for flow cytometry. Quantification of the percentage of B2M-negative and CD3-negative cells is shown in FIG. 8A for gRNAs with a repeat length of 20 nucleotides and a spacer length of 20 nucleotides, and in FIG. 8B for gRNAs with a repeat length of 20 nucleotides and a spacer length of 17 nucleotides. Corresponding flow cytometry panels can be seen in FIG. 8C for gRNAs of different repeat and spacer lengths and with different modifications.

In a further study, RNP complexes were formed using CasΦ.12 and modified gRNAs (unmodified, line, 2me, 3me, 2′-fluoro on the last 3′ nucleotide of the crRNA (RF), 2′-fluoro on the last two 3′ nucleotides of the crRNA (2F) and 2′-fluoro on the last three 3′ nucleotides of the crRNA (3F)) with different lengths of spacer sequences (20-20 and 20-17 as above) that target TRAC. T cells were nucleofected with RNP complexes (125 pmol) using the P3 primary cell nucleofection kit and an Amaxa 4D 96-well electroporation system with pulse code EH115. As shown in FIG. 8D, ˜90 % editing efficiency was achieved using CasΦ.12 and modified gRNAs. FIG. 8E shows a flow cytometry plot illustrating-90% TRAC knockout in T cells after delivery of CasΦ.12 and modified gRNAs. This data further demonstrates the ability of CasΦ to mediate high efficiency genome editing.

TABLE 18

| | | | Repeat | Spacer | |
|---|---|---|---|---|---|
| | | | sequence | sequence | crRNA sequence |
| Name | Target | Modification | (5′ --> 3′) | (5′ --> 3′) | (5′ --> 3′) |
| R3150 | B2M | Unmodified, 2′OMe | AUUGCUCC | CAGUGGGG | AUUGCUCCUUA |
| 20-20 | Exon 2 | at last 3′ base (1me) | UUACGAG | GUGAAUUC | CGAGGAGACCA |
| | | 2′OMe at last two | GAGAC | AGUG (SEQ | GUGGGGGUGAA |
| | | 3′ bases (2me) | (SEQ ID | ID NO: 1351) | UUCAGUG (SEQ |
| | | 2′OMe at last three | NO: 1350) | | ID NO: 1352) |
| | | 3′ bases (3me) | | | |
| R3042 | TRAC | Unmodified, | AUUGCUCC | GAGUCUCU | AUUGCUCCUUA |
| 20-20 | Exon 1 | 1me | UUACGAG | CAGCUGGU | CGAGGAGACGA |
| | | 2me | GAGAC | ACAC (SEQ | GUCUCUCAGCU |
| | | 3me | (SEQ ID | ID NO: 1353) | GGUACAC (SEQ |
| | | | NO: 1350) | | ID NO: 1354) |
| R3150 | B2M | Unmodified, | AUUGCUCC | CAGUGGGG | AUUGCUCCUUA |
| 20-17 | Exon 2 | 1me | UUACGAG | GUGAAUUC | CGAGGAGACCA |
| | | 2me | GAGAC | A (SEQ ID | GUGGGGGUGAA |
| | | 3me | (SEQ ID | NO: 1355) | UUCA (SEQ ID |
| | | | NO: 1350) | | NO: 1356) |
| R3042 | TRAC | Unmodified, | AUUGCUCC | CAGUGGGG | AUUGCUCCUUA |
| 20-17 | Exon 1 | 1me | UUACGAG | GUGAAUUC | CGAGGAGACGA |
| | | 2me | GAGAC | A (SEQ ID | GUCUCUCAGCU |
| | | 3me | (SEQ ID | NO: 1355) | GGUA (SEQ ID |
| | | | NO: 1350) | | NO: 1357) |

### Example 7. Identification of Optimal Guide RNAs for CasΦ Polypeptide-Mediated Genome Editing in Primary Cells

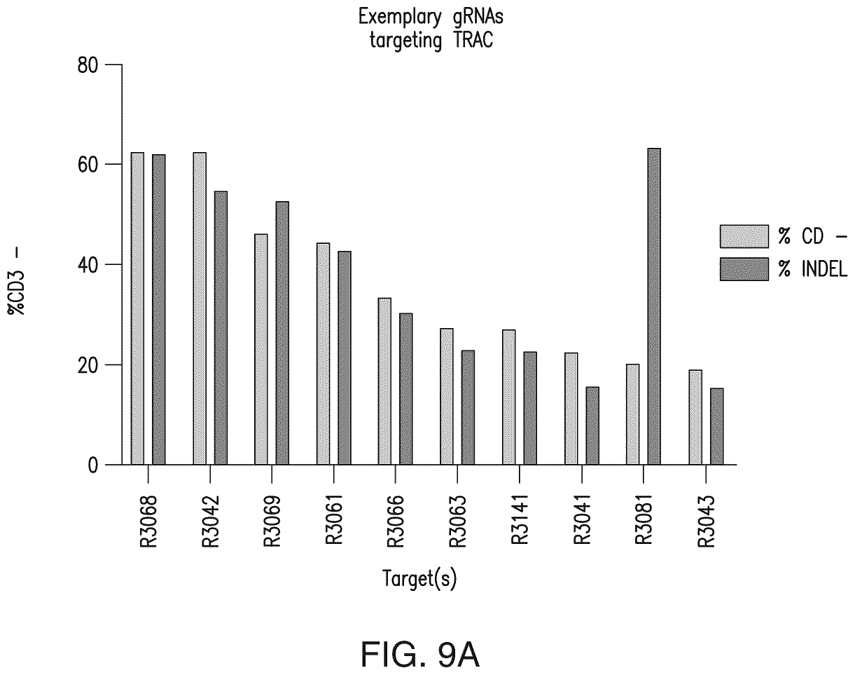

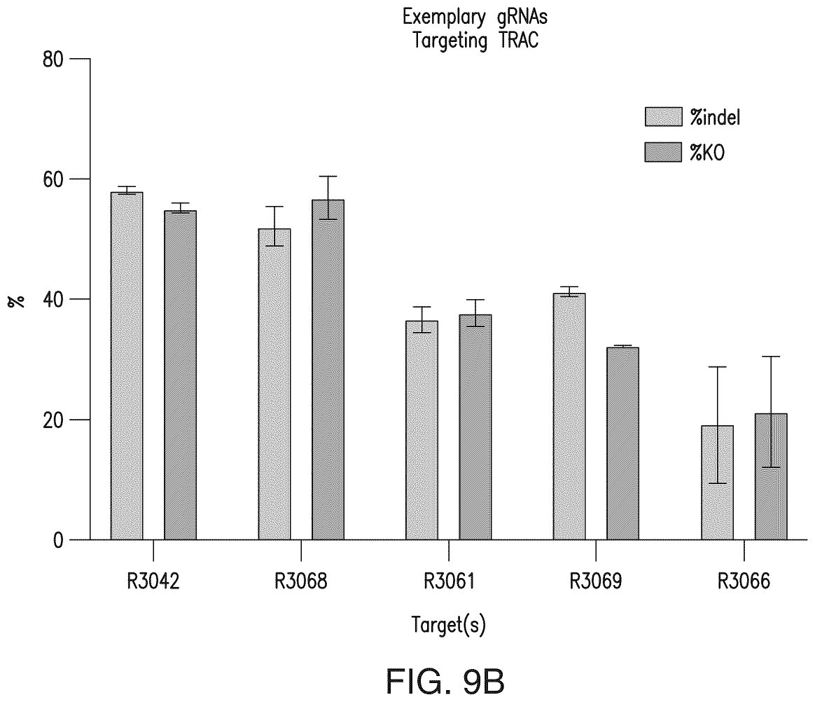

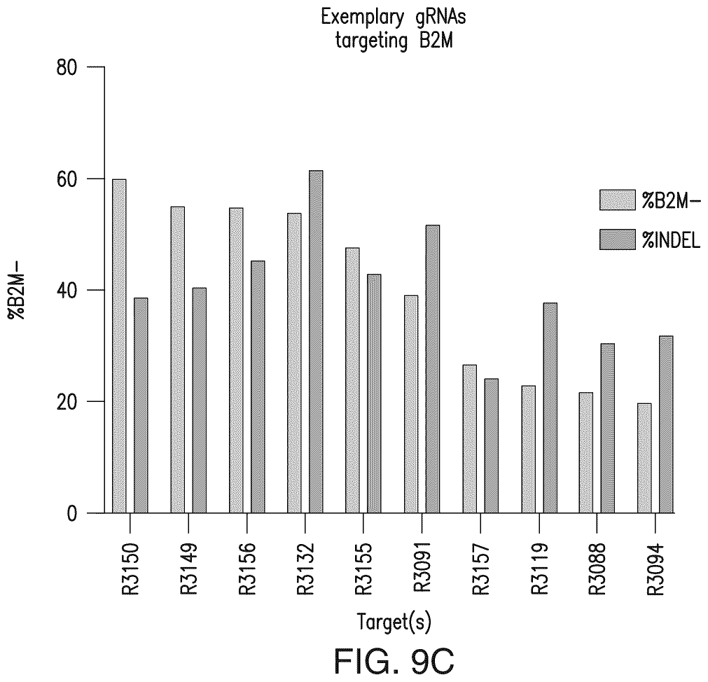

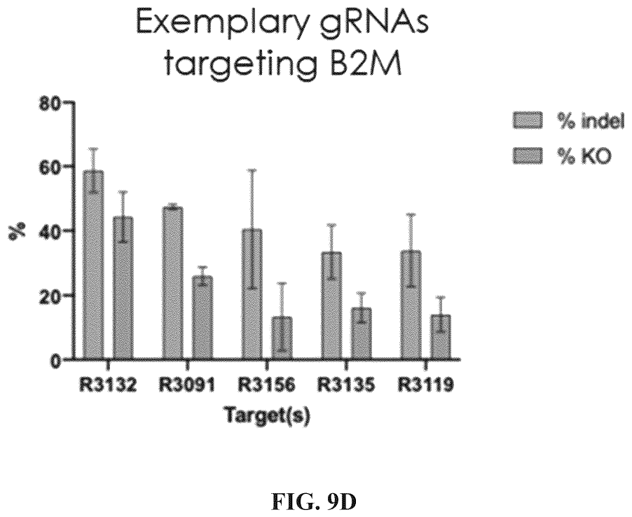

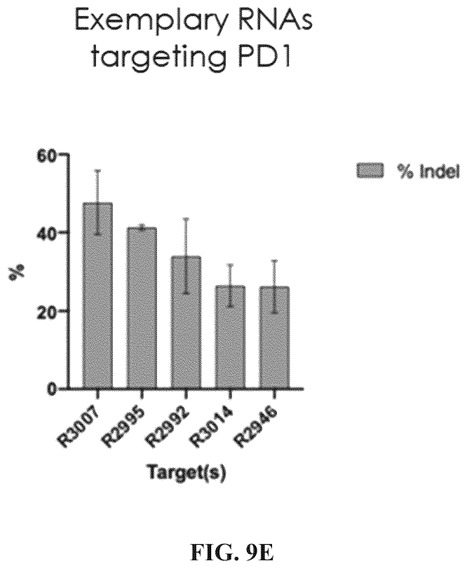

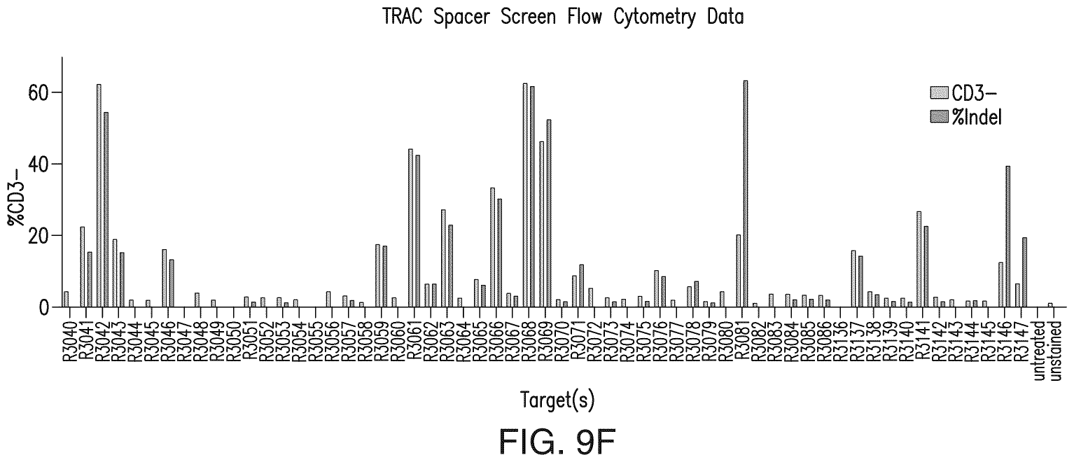

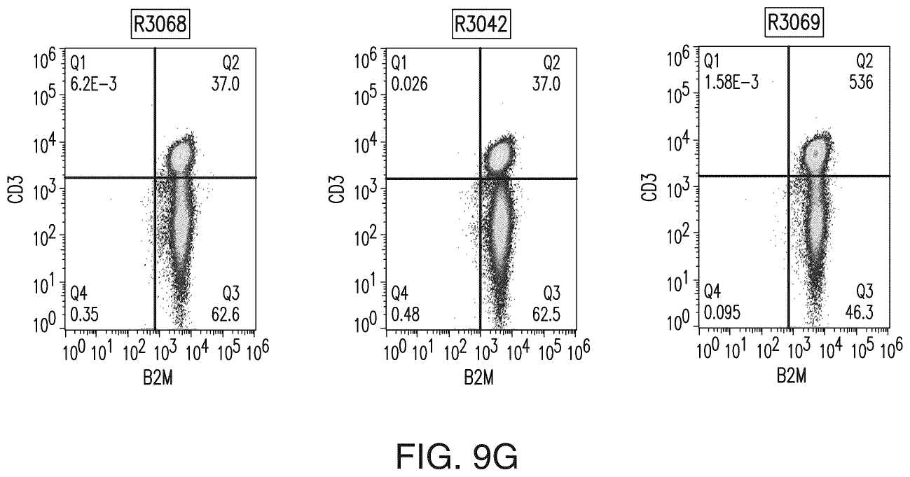

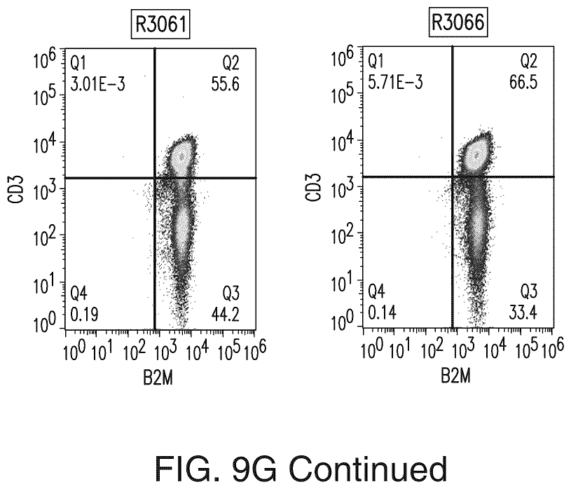

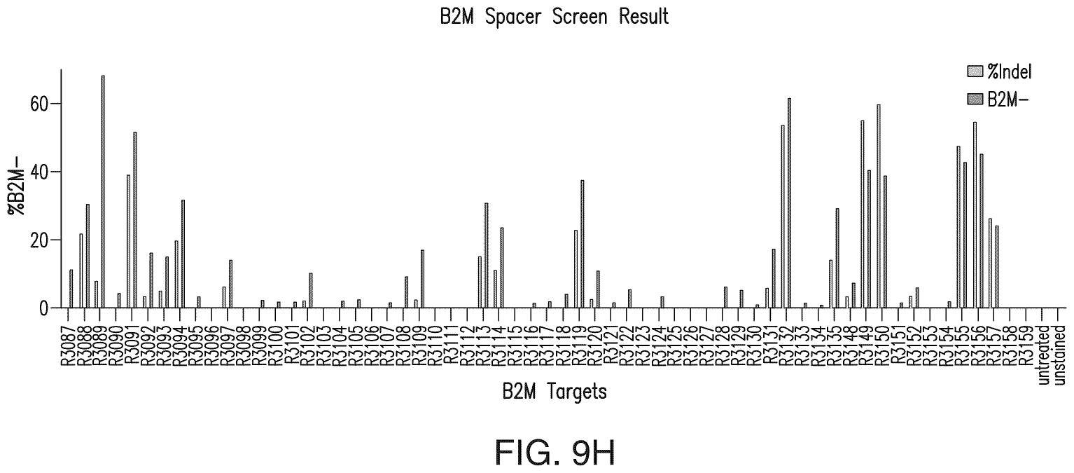

The present example shows identification of the best performing gRNAs that target TRAC, B32M and programmed cell death protein 1 (PD1) in T cells. In this study, CasΦ.12 polypeptides (SEQ ID NO: 57) were incubated with different gRNAs (shown in TABLE 19) at room temperature for 10 minutes to form RNP complexes. T cells were resuspended at 5×10⁵ cells/20 μL in electroporation solution (Lonza) and an Amaxa 4D Nucleofector with pulse code EH15 was used to nucleofect the cells Immediately after nucleofection, 80 d pre-warmed culture medium was added to each well. The cells were then left in the cuvette plate for 10 minutes before transfer to the culture plate. After 48 hours, DNA was extracted from half of the cells and PCR was performed to detect the frequency of indels. The rest of the cells were cultured until Day 5, and were then collected for flow cytometry to detect the frequency of TRAC or 2M knockout. FIG. 9A and FIG. 9B show exemplary gRNAs for targeting TRAC. FIG. 9C and FIG. 9D show exemplary gRNAs for targeting B32M. FIG. 9E shows exemplary gRNAs for targeting PD 1. Additionally, this example demonstrates that a guide RNAs targeting a non-coding region can mediate gene knockout. For example, R3007, R2995, R2992 and R3014 target non-coding regions of the PD1 gene. The screening for gRNAs targeting TRAC is shown in FIG. 9F and for gRNAs targeting B2M is shown in FIG. 9H. Flow cytometry plots of exemplary gRNAs targeting TRAC are shown in FIG. 9G and of exemplary gRNAs targeting B32M in FIG. 9I.

TABLE 19

| Name | Target | Spacer sequence (5′ -- > 3′) |
|---|---|---|
| R3041 | TRAC | UCCCACAGAUAUCCAGAACC (SEQ ID NO: 1358) |
| R3042 | TRAC | GAGUCUCUCAGCUGGUACAC (SEQ ID NO: 1353) |
| R3043 | TRAC | AGAGUCUCUCAGCUGGUACA (SEQ ID NO: 1359) |
| R3061 | TRAC | AAGUCCAUAGACCUCAUGUC (SEQ ID NO: 1360) |
| R3063 | TRAC | AAGAGCAACAGUGCUGUGGC (SEQ ID NO: 1361) |
| R3066 | TRAC | GUUGCUCCAGGCCACAGCAC (SEQ ID NO: 1362) |
| R3068 | TRAC | GCACAUGCAAAGUCAGAUUU (SEQ ID NO: 1363) |
| R3069 | TRAC | GCAUGUGCAAACGCCUUCAA (SEQ ID NO: 1364) |
| R3081 | TRAC | CUAAAAGGAAAAACAGACAU (SEQ ID NO: 1365) |
| R3141 | TRAC | CUCGACCAGCUUGACAUCAC (SEQ ID NO: 1366) |
| R3088 | B2M | AUAUAAGUGGAGGCGUCGCG (SEQ ID NO: 1367) |
| R3091 | B2M | GGGCCGAGAUGUCUCGCUCC (SEQ ID NO: 1368) |
| R3094 | B2M | UGGCCUGGAGGCUAUCCAGC (SEQ ID NO: 1369) |
| R3119 | B2M | AAGUUGACUUACUGAAGAAU (SEQ ID NO: 1370) |
| R3132 | B2M | AGCAAGGACUGGUCUUUCUA (SEQ ID NO: 1371) |
| R3149 | B2M | AGUGGGGGUGAAUUCAGUGU (SEQ ID NO: 1372) |
| R3150 | B2M | CAGUGGGGGUGAAUUCAGUG (SEQ ID NO: 1351) |
| R3155 | B2M | GGCUGUGACAAAGUCACAUG (SEQ ID NO: 1373) |
| R3156 | B2M | GUCACAGCCCAAGAUAGUUA (SEQ ID NO: 1374) |
| R3157 | B2M | UCACAGCCCAAGAUAGUUAA (SEQ ID NO: 1375) |
| R2946 | PD1 | UGUGACACGGAAGCGGCAGU (SEQ ID NO: 1376) |
| R2992 | PD1 | GGGGCUGGUUGGAGAUGGCC (SEQ ID NO: 1377) |
| R2995 | PD1 | GAGCAGCCAAGGUGCCCCUG (SEQ ID NO: 1378) |
| R3007 | PD1 | ACACAUGCCCAGGCAGCACC (SEQ ID NO: 1379) |
| R3014 | PD1 | AGGCCCAGCCAGCACUCUGG (SEQ ID NO: 1380) |

### Example 8. RNP and mRNA Delivery of CasΦ Polypeptides

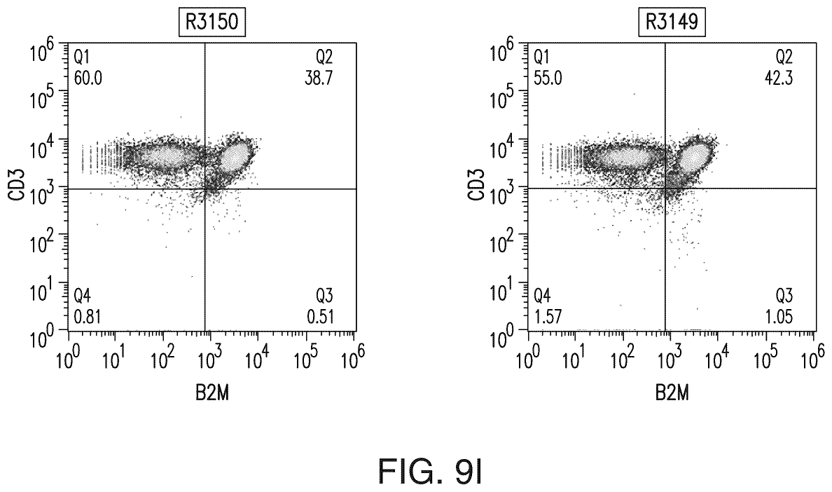

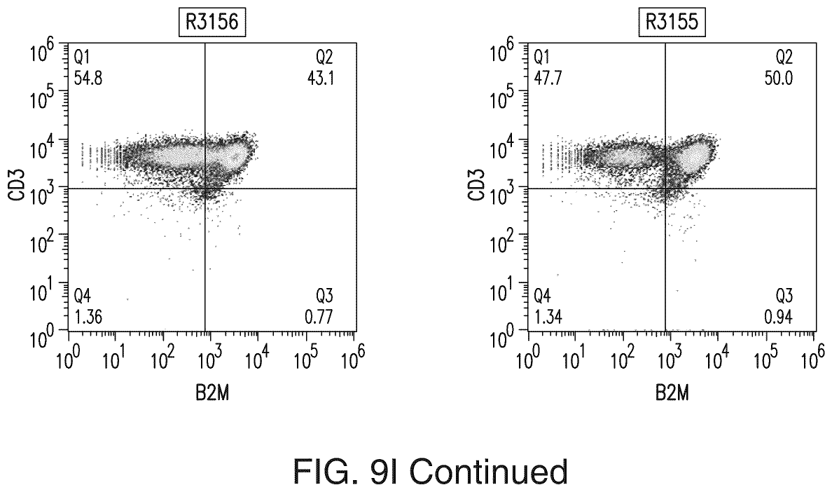

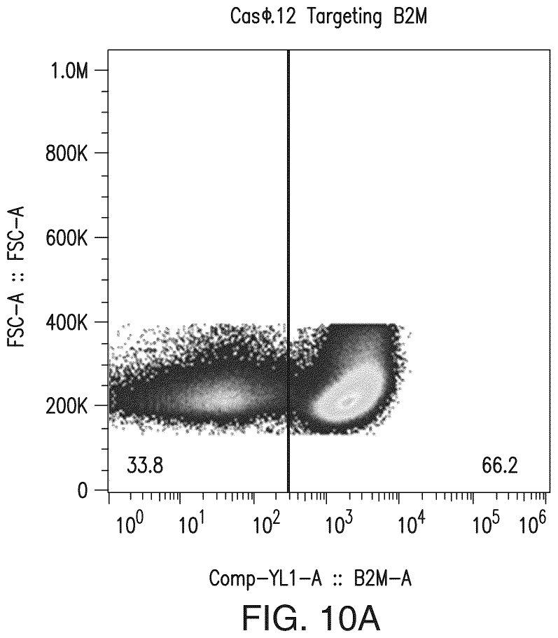

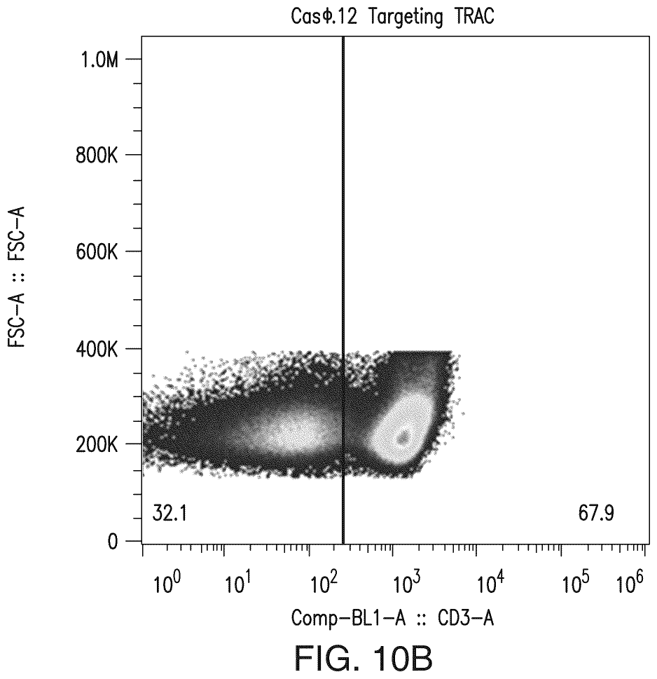

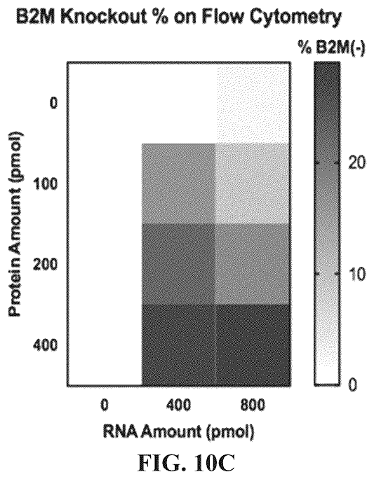

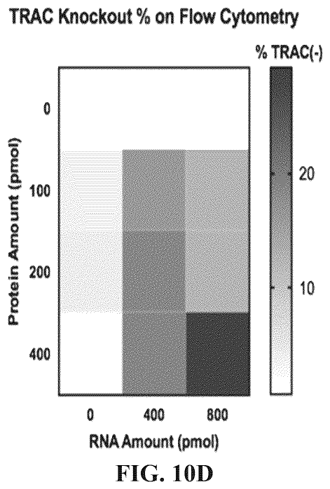

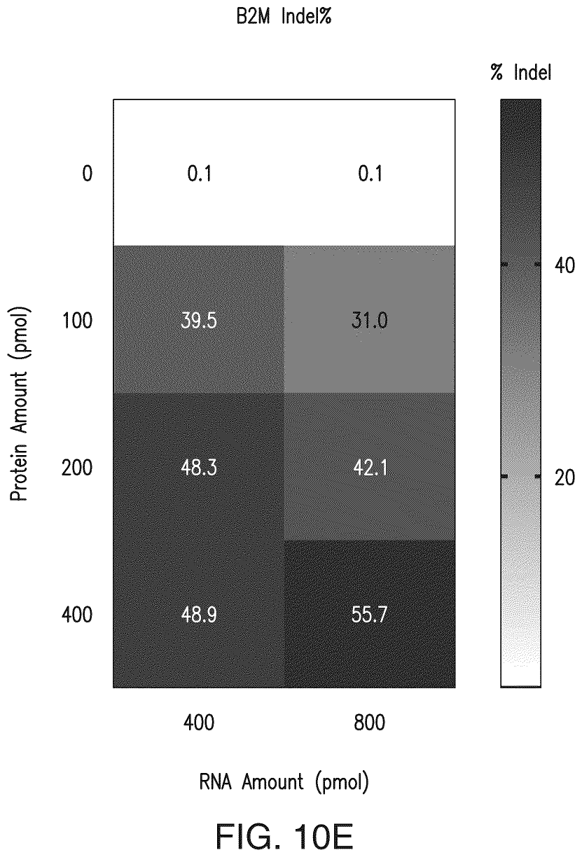

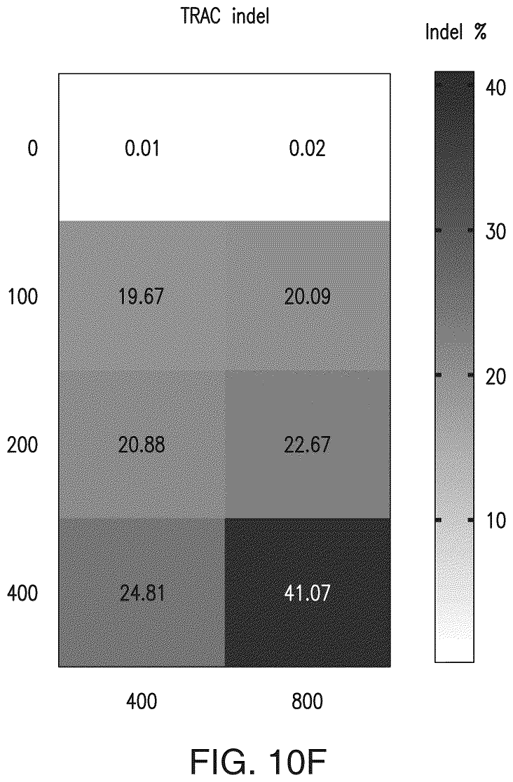

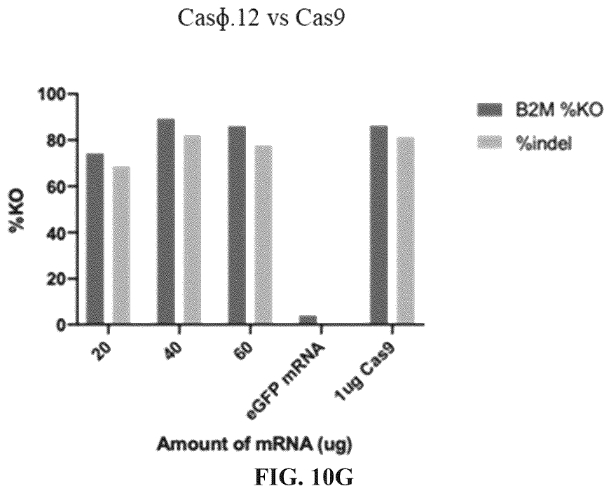

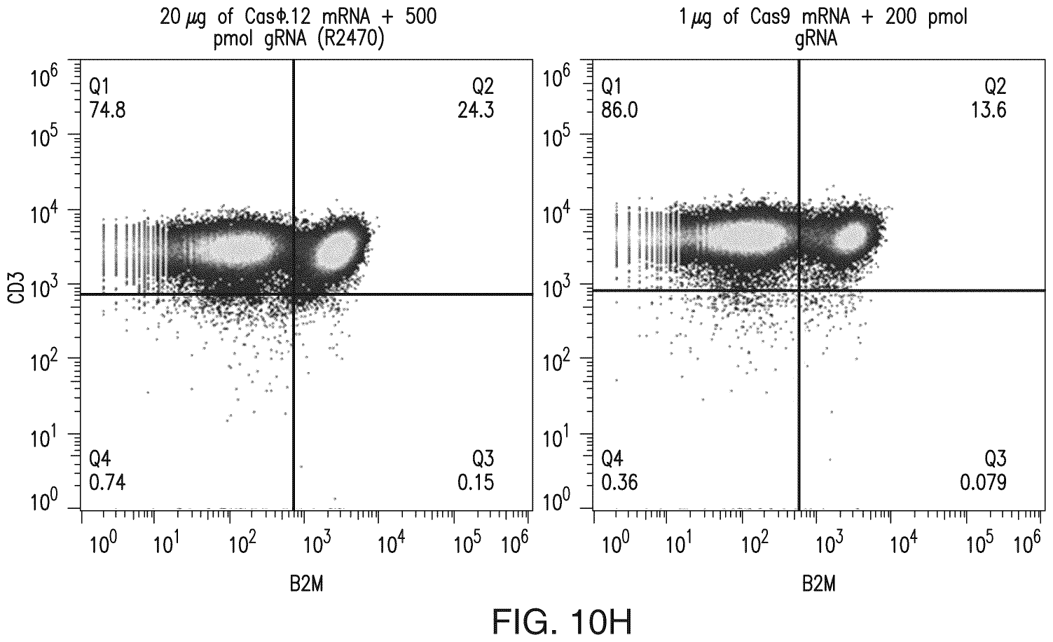

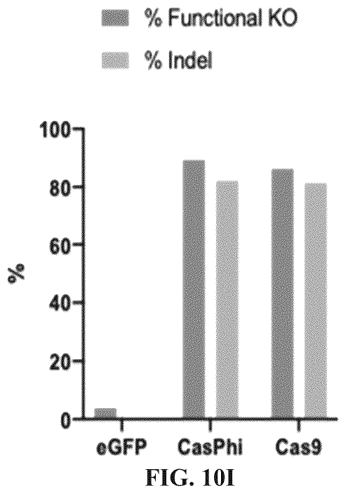

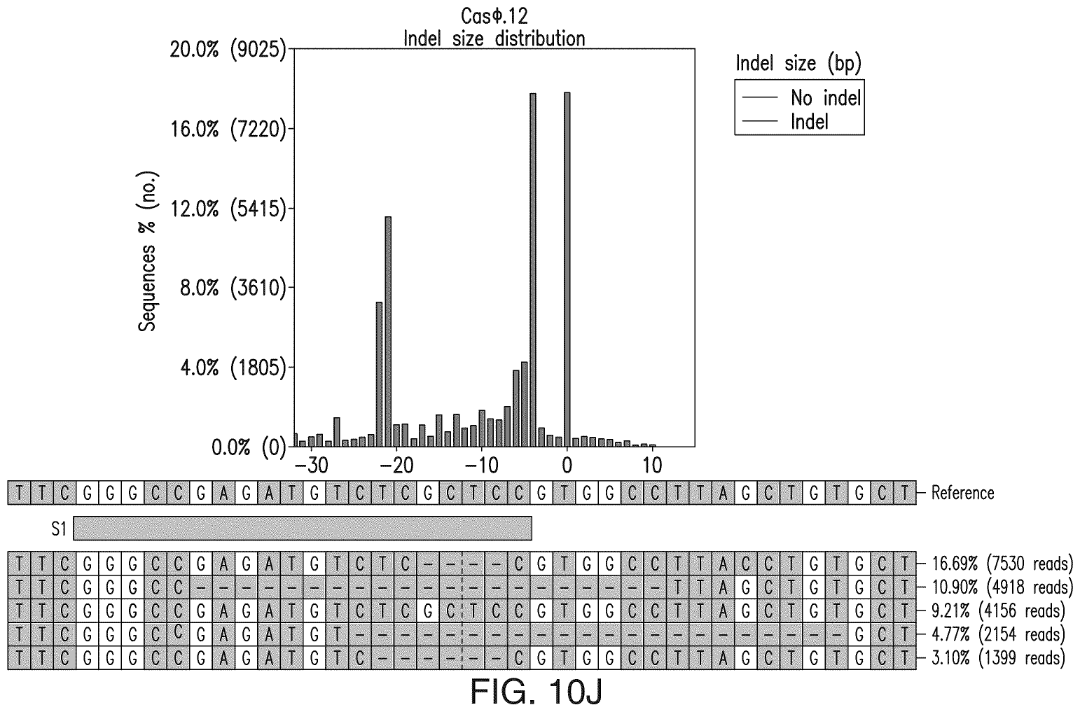

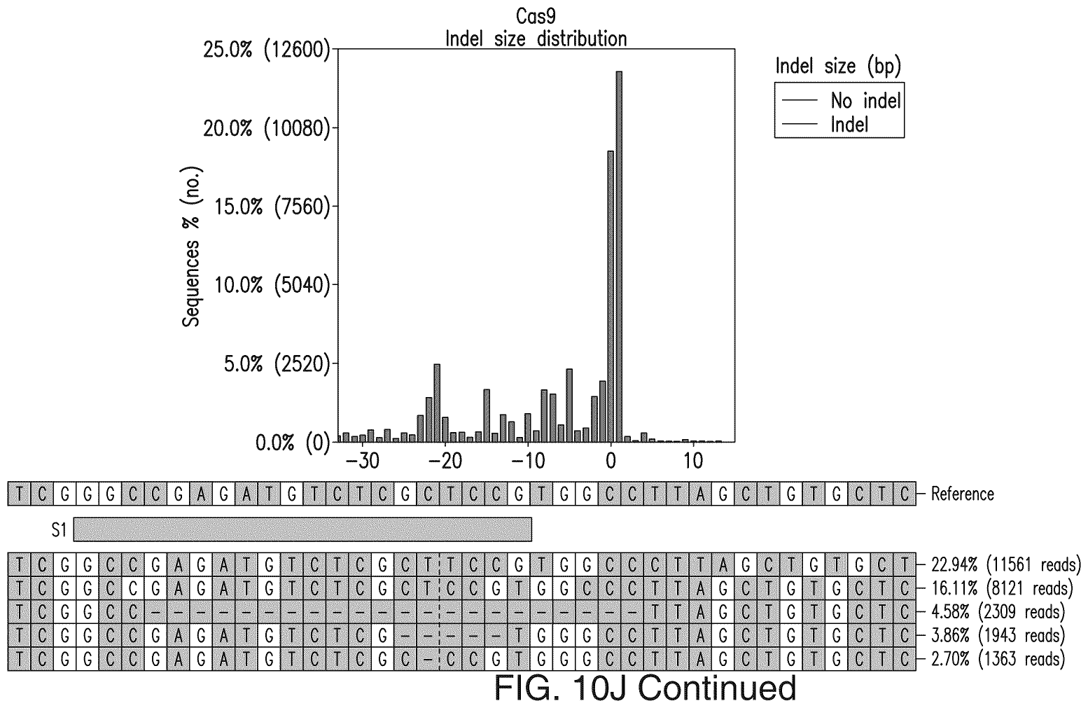

This example illustrates that CasΦ.12 can be delivered to primary cells as mRNA or as an RNP complex. In one study, RNP complexes were formed using CasΦ.12 protein (0, 100, 200 or 400 μmol) (SEQ ID NO: 57) and gRNAs (0, 400 or 800 μmol) targeting B2M or TRAC. RNP complexes were added to T cells. T cells were nucleofected using the Amaxa P3 kit and Amaxa 4D 96-well electroporation system with pulse code EH115. Cells were harvested for flow cytometry to determine the percentage of B2M or TRAC knockout cells, and genomic DNA was extracted to detect the frequency of indel mutations. As shown in FIG. 10A, a distinct population of B2M-negative cells was detected in T cells transfected with CasΦ.12 RNP complex targeting B2M. A distinct population of TRAC-negative cells was detected in in T cells transfected with CasΦ.12 RNP complex targeting TRAC, and shown in FIG. 10B. Quantification of the percentage of B2M knockout cells is shown in FIG. 10C and quantification of the percentage of TRAC knockout cells is shown in FIG. 10D. A high frequency of indel mutations was also seen after delivery of RNP complexes. As shown in FIG. 10E, ˜55% indel mutations was detected when RNP complexes targeting B2M were formed using 400 pmol protein and 800 pmol guide RNA. A similar frequency of indel mutations was detected when RNP complexes targeting TRAC were formed using the same conditions, as illustrated in FIG. 10F.

In a second study, CasΦ.12 mRNA was delivered to T cells with a gRNA targeting the B2M gene. For nucleofection, T cells were resuspended in BTXpress electroporation medium (5×10⁵ cells per well) and mixed with CasΦ.12 mRNA and 500 pmol gRNA. Cells were collected on Day 2 for extraction of genomic DNA, and the frequency of indel mutations was determined. As shown in FIG. 10G and FIG. 10H, delivery of CasΦ.12 mRNA and gRNA resulted in a high frequency of indel mutations. This was at a comparable level to genome editing with delivery of Cas9 mRNA. Further data from this study are shown in FIG. 10I and FIG. 10J. FIG. 10I shows the frequency of indel mutations and functional knockout, as assessed by flow cytometry, of the B2M gene induced by either CasΦ.12 or Cas9 targeting the same site. FIG. 10J shows the distribution of the size of indel mutations induced by CasΦ.12 or Cas9 determined by NGS analysis. CasΦ.12 predominantly induced larger deletion mutations whereas Cas9 induced mostly small 1 bp InDels. This data further confirms the ability of CasΦ.12 to mediate genome editing at the B2M locus.

### Example 9. Multiplex Genome Editing with CasΦ Polypeptides

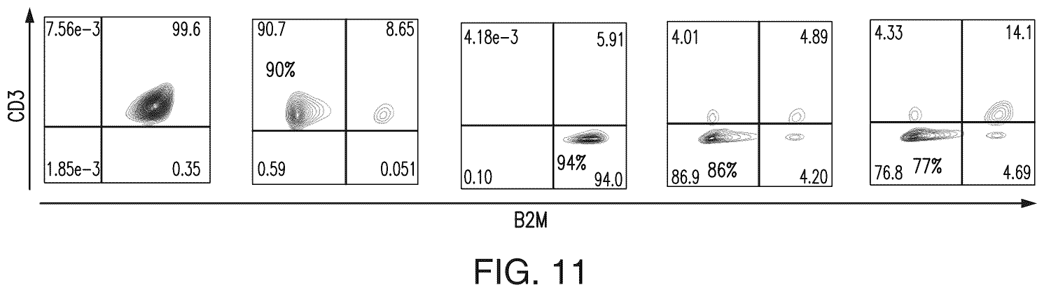

This example illustrates the ability of CasΦ RNP complexes to knockout multiple genes simultaneously. In this study, gRNAs targeting B2M, TRAC and PDCD1 (provided in TABLE 20) were incubated with CasΦ.12 (SEQ ID NO: 57) for 10 minutes at room temperature to form B32M, TRAC, and PDC1 targeting RNPs, respectively. The 2M targeting RNPs, TRAC targeting RNPs, PDCD1 targeting RNPs and combinations thereof were added to T cells. T cells were resuspended at 5×10⁵ cells/20 μL in Nucleofection P3 solution and an Amaxa4D 96-well electroporation system with pulse code EH115 was used to nucleofect the cells. Immediately after nucleofection, 85 d pre-warmed culture medium was added to each well. The cells were then left in the cuvette plate for 10 minutes before transfer to the culture plate. On Day 3, genomic DNA was extracted and sent for NGS sequencing and the 0 indel was measured with a positive indel being indicative of 0% knockout. On Day 5, cells were harvested for flow cytometry and the 00 knockout was measured with fluorescently labeled antibodies to TRAC and 82M (antibody to PDCD1 unavailable). % indel results are presented in TABLE 21 and flow cytometry data presented in TABLE 22. Corresponding flow cytometry panels are shown in FIG. 11.

TABLE 20

| Descrip- | SEQ | |
|---|---|---|
| tion | ID | gRNA Sequence |
| B2M gRNA | 1381 | CUUUCAAGACUAAUAGAUUGCUCCUUACGAGGAG |
| (R3132) | | ACAGCAAGGACUGGUCUUUCUA |
| TRAC gRNA | 1382 | CUUUCAAGACUAAUAGAUUGCUCCUUACGAGGAG |
| (R3042) | | ACGAGUCUCUCAGCUGGUACAC |
| PDCD1 gRNA | 1383 | CUUUCAAGACUAAUAGAUUGCUCCUUACGAGGAG |
| (R2925) | | ACUAGCACCGCCCAGACGACUG |

TABLE 21

| Description | RNP Guide ID(s) | Amplicon | % INDEL |
|---|---|---|---|
| TRAC single KO | R3042 | TRAC | 77.6% |
| B2M single KO | R3132 | B2M | 85.5% |
| PDCD1 single KO | R2925 | PDCD1 | 44.6% |
| TRAC, B2M double KO | R3132 & R3042 | TRAC | 58.8% |
| TRAC, B2M double KO | R3132 & R3042 | B2M | 61.2% |
| TRAC, B2M, PDCD1 triple KO | R3132, R3042, R2925 | TRAC | 59.2% |
| TRAC, B2M, PDCD1 triple KO | R3132, R3042, R2925 | B2M | 69.4% |
| TRAC, B2M, PDCD1 triple KO | R3132, R3042, R2925 | PDCD1 | 42.1% |

TABLE 22

| gRNA | B2M+ CD3− | B2M+, CD3+ | B2M−, CD3+ | B2M−, CD3− |
|---|---|---|---|---|
| TRAC | 94 | 5.91 | 0.00418 | 0.1 |
| B2M | 0.051 | 8.65 | 90.7 | 0.59 |
| TRAC + B2M | 4.2 | 4.89 | 4.01 | 86.9 |
| TRAC + B2M + | 4.74 | 14.1 | 4.33 | 76.8 |
| PDCD1 | | | | |

### Example 10. Adeno-Associated Virus Encoding CasΦ.12 Facilitates Genome Editing

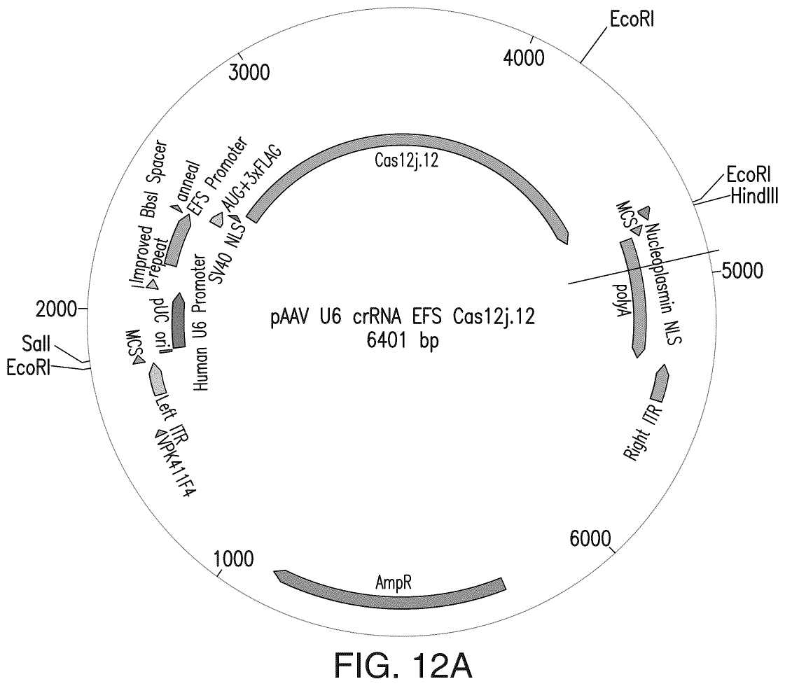

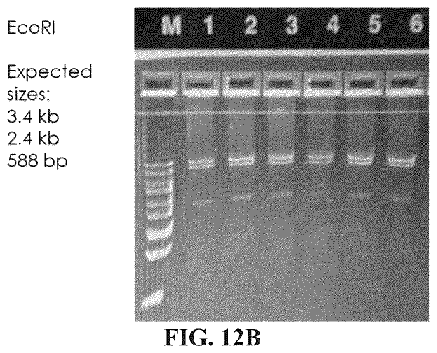

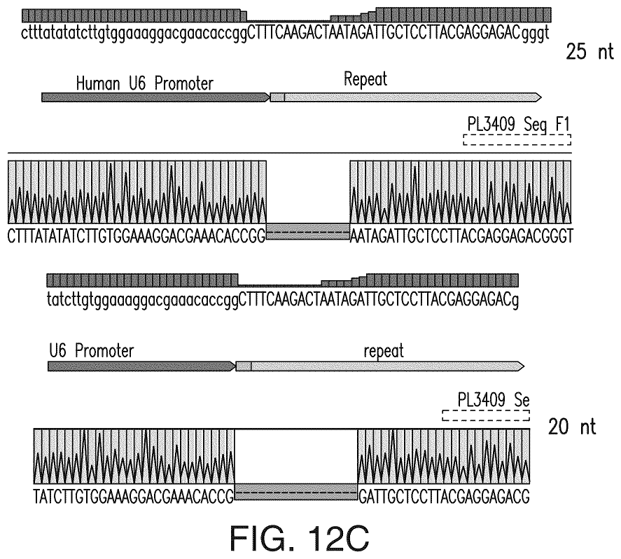

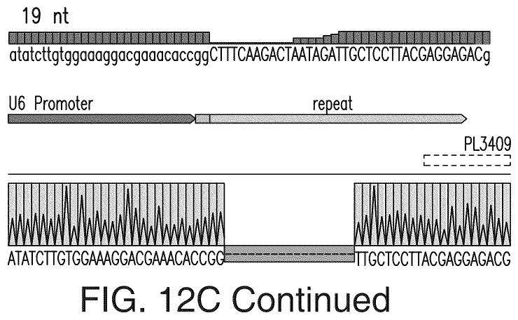

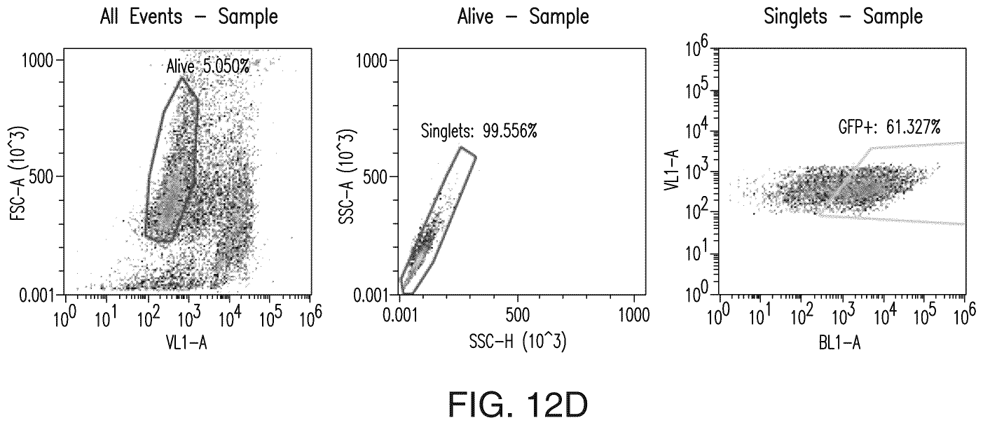

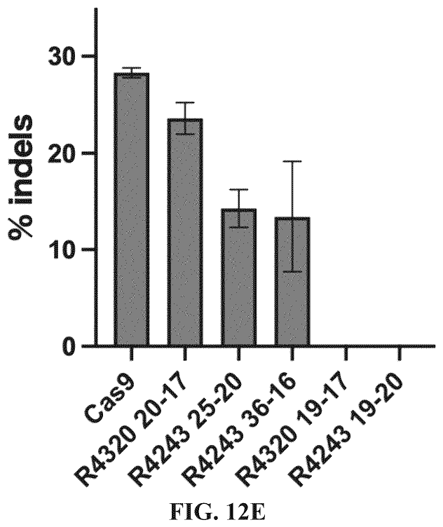

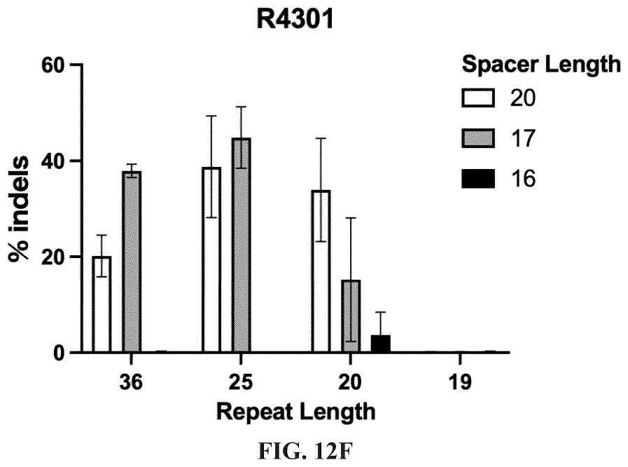

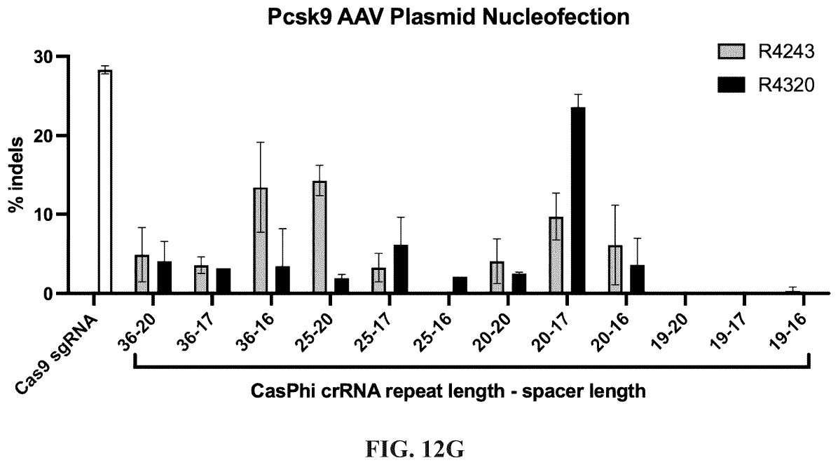

This example shows that a CasΦ.12 plasmid, including both CasΦ polypeptide sequence and gRNA sequence, sometimes called an all-in-one, can be used to facilitate genome editing. In this study, the crRNAs (sequences shown in TABLE 23 and TABLE 24) from the initial RNP screen were chosen and truncations of these crRNAs were generated with repeat lengths of 36, 25, 20, or 19 nucleotides in combination with spacer lengths of 20, 17, or 16 nucleotides. Each crRNA was then cloned into an AAV vector consisting of U6 promoter to drive crRNA expression, intron-less EF1alpha short (EFS) promoter driving CasΦ expression, PolyA signal, and 1 kb stuffer sequence genomic. Hepal-6 mouse hepatoma cells were nucleofected with 10 μg of each AAV plasmid. After 72 hours, genomic DNA was extracted and the frequency of indel mutations was determined using NGS. FIG. 12A shows a plasmid map of the adeno-associated virus (AAV) encoding the CasΦ polypeptide sequence and gRNA sequence. FIG. 12B illustrates repeat truncations. FIG. 12C shows various truncated repeat sequences (25 nt, 20 nt and 19 nt), the data of which shown in FIGS. 12D-12G. FIG. 12D shows efficient transfection with AAV. FIG. 12E shows the frequency of CasΦ.12 induced indel mutations in Hepal-6 cells transduced with 10 μg of each AAV plasmid. gRNAs containing repeat sequences of 19, 20, 25 or 36 nucleotides and spacer sequences of 16, 17 or 20 nucleotides were used in this study. In the graph legend, repeat and spacer lengths are indicated as the number of nucleotides in the repeat followed by the number of nucleotides in the spacer, e.g. 20-17 has a repeat length of 20 nucleotides and a spacer length of 17 nucleotides. The frequency of indel mutations is comparable to that of Cas9. FIG. 12F, and FIG. 12G show the frequency of CasΦ.12 induced indel mutations with different gRNA containing repeat and spacer sequences of different lengths (indicated as in FIG. 12F with repeat length followed by spacer length). This study demonstrates that the all-in-one vector method of CasΦ.12 mediated genome editing is robust across different gRNA sequences and with gRNAs of different repeat and spacer lengths.

TABLE 23
spacer sequences of gRNAs targeting mouse PCSK9

| | | | SEQ ID |
|---|---|---|---|
| Name | Spacer sequence (5′ --> 3′) | Target | NO |
| R4238 | CCGCUGUUGCCGCCGCUGCU | PCSK9 | 1384 |
| R4239 | CCGCCGCUGCUGCUGCUGUU | PCSK9 | 1385 |
| R4240 | CUGCUACUGUGCCCCACCGG | PCSK9 | 1386 |
| R4241 | AUAAUCUCCAUCCUCGUCCU | PCSK9 | 1387 |
| R4242 | UGAAGAGCUGAUGCUCGCCC | PCSK9 | 1388 |
| R4243 | GAGCAACGGCGGAAGGUGGC | PCSK9 | 1389 |
| R4244 | CUGGCAGCCUCCAGGCCUCC | PCSK9 | 1390 |
| R4245 | UGGUGCUGAUGGAGGAGACC | PCSK9 | 1391 |
| R4246 | AAUCUGUAGCCUCUGGGUCU | PCSK9 | 1392 |
| R4247 | UUCAAUCUGUAGCCUCUGGG | PCSK9 | 1393 |
| R4248 | GUUCAAUCUGUAGCCUCUGG | PCSK9 | 1394 |
| R4249 | AACAAACUGCCCACCGCCUG | PCSK9 | 1395 |
| R4250 | AUGACAUAGCCCCGGCGGGC | PCSK9 | 1396 |
| R4251 | UACAUAUCUUUUAUGACCUC | PCSK9 | 1397 |
| R4252 | UAUGACCUCUUCCCUGGCUU | PCSK9 | 1398 |
| R4253 | AUGACCUCUUCCCUGGCUUC | PCSK9 | 1399 |
| R4254 | UGACCUCUUCCCUGGCUUCU | PCSK9 | 1400 |
| R4255 | ACCAAGAAGCCAGGGAAGAG | PCSK9 | 1401 |
| R4256 | CCUGGCUUCUUGGUGAAGAU | PCSK9 | 1402 |
| R4257 | UUGGUGAAGAUGAGCAGUGA | PCSK9 | 1403 |
| R4258 | GUGAAGAUGAGCAGUGACCU | PCSK9 | 1404 |
| R4259 | CCCCAUGUGGAGUACAUUGA | PCSK9 | 1405 |
| R4260 | CUCAAUGUACUCCACAUGGG | PCSK9 | 1406 |
| R4261 | AGGAAGACUCCUUUGUCUUC | PCSK9 | 1407 |
| R4262 | GUCUUCGCCCAGAGCAUCCC | PCSK9 | 1408 |
| R4263 | UCUUCGCCCAGAGCAUCCCA | PCSK9 | 1409 |
| R4264 | GCCCAGAGCAUCCCAUGGAA | PCSK9 | 1410 |
| R4265 | CAUGGGAUGCUCUGGGCGAA | PCSK9 | 1411 |
| R4266 | GCUCCAGGUUCCAUGGGAUG | PCSK9 | 1412 |
| R4267 | UCCCAGCAUGGCACCAGACA | PCSK9 | 1413 |
| R4268 | CUCUGUCUGGUGCCAUGCUG | PCSK9 | 1414 |
| R4269 | GAUACCAGCAUCCAGGGUGC | PCSK9 | 1415 |
| R4270 | AGGGCAGGGUCACCAUCACC | PCSK9 | 1416 |
| R4271 | AAGUCGGUGAUGGUGACCCU | PCSK9 | 1417 |
| R4272 | AACAGCGUGCCGGAGGAGGA | PCSK9 | 1418 |
| R4273 | GCCACACCAGCAUCCCGGCC | PCSK9 | 1419 |
| R4274 | AGCACACGCAGGCUGUGCAG | PCSK9 | 1420 |
| R4275 | ACAGUUGAGCACACGCAGGC | PCSK9 | 1421 |
| R4276 | CCUUGACAGUUGAGCACACG | PCSK9 | 1422 |
| R4277 | GCUGACUCUUCCGAAUAAAC | PCSK9 | 1423 |
| R4278 | AUUCGGAAGAGUCAGCUAAU | PCSK9 | 1424 |
| R4279 | UUCGGAAGAGUCAGCUAAUC | PCSK9 | 1425 |
| R4280 | GGAAGAGUCAGCUAAUCCAG | PCSK9 | 1426 |
| R4281 | UGCUGCCCCUGGCCGGUGGG | PCSK9 | 1427 |
| R4282 | AGGAUGCGGCUAUACCCACC | PCSK9 | 1428 |
| R4283 | CCAGCUGCUGCAACCAGCAC | PCSK9 | 1429 |
| R4284 | CAGCAGCUGGGAACUUCCGG | PCSK9 | 1430 |
| R4285 | CGGGACGACGCCUGCCUCUA | PCSK9 | 1431 |
| R4286 | GUGGCCCCGACUGUGAUGAC | PCSK9 | 1432 |
| R4287 | CCUUGGGGACUUUGGGGACU | PCSK9 | 1433 |
| R4288 | GUCCCCAAAGUCCCCAAGGU | PCSK9 | 1434 |
| R4289 | GGGACUUUGGGGACUAAUUU | PCSK9 | 1435 |
| R4290 | GGGGACUAAUUUUGGACGCU | PCSK9 | 1436 |
| R4291 | GGGACUAAUUUUGGACGCUG | PCSK9 | 1437 |
| R4292 | UGGACGCUGUGUGGAUCUCU | PCSK9 | 1438 |
| R4293 | GGACGCUGUGUGGAUCUCUU | PCSK9 | 1439 |
| R4294 | GACGCUGUGUGGAUCUCUUU | PCSK9 | 1440 |
| R4295 | CCGGGGGCAAAGAGAUCCAC | PCSK9 | 1441 |
| R4296 | GCCCCCGGGAAGGACAUCAU | PCSK9 | 1442 |
| R4297 | CCCCCGGGAAGGACAUCAUC | PCSK9 | 1443 |
| R4298 | AUGUCACAGAGUGGGACCUC | PCSK9 | 1444 |
| R4299 | UGGCUCGGAUGCUGAGCCGG | PCSK9 | 1445 |
| R4300 | CCCUGGCCGAGCUGCGGCAG | PCSK9 | 1446 |
| R4301 | GUAGAGAAGUGGAUCAGCCU | PCSK9 | 1447 |
| R4302 | GGUAGAGAAGUGGAUCAGCC | PCSK9 | 1448 |
| R4303 | UCUACCAAAGACGUCAUCAA | PCSK9 | 1449 |
| R4304 | AUGACGUCUUUGGUAGAGAA | PCSK9 | 1450 |
| R4305 | CCUGAGGACCAGCAGGUGCU | PCSK9 | 1451 |
| R4306 | GGGGUCAGCACCUGCUGGUC | PCSK9 | 1452 |
| R4307 | GAGUGGGCCCCGAGUGUGCC | PCSK9 | 1453 |
| R4308 | UGGGGCACAGCGGGCUGUAG | PCSK9 | 1454 |
| R4309 | UCCAGGAGCGGGAGGCGUCG | PCSK9 | 1455 |
| R4310 | CAGACCUGCUGGCCUCCUAU | PCSK9 | 1456 |
| R4311 | AGGGCCUUGCAGACCUGCUG | PCSK9 | 1457 |
| R4312 | GGGGGUGAGGGUGUCUAUGC | PCSK9 | 1458 |
| R4313 | GGGGUGAGGGUGUCUAUGCC | PCSK9 | 1459 |
| R4314 | GCACGGGGAACCAGGCAGCA | PCSK9 | 1460 |
| R4315 | CCCGUGCCAACUGCAGCAUC | PCSK9 | 1461 |
| R4316 | UGGAUGCUGCAGUUGGCACG | PCSK9 | 1462 |
| R4317 | UGGUGGCAGUGGACAUGGGU | PCSK9 | 1463 |
| R4318 | CACUUCCCAAUGGAAGCUGC | PCSK9 | 1464 |
| R4319 | CAUUGGGAAGUGGAAGACCU | PCSK9 | 1465 |
| R4320 | GGAAGUGGAAGACCUUAGUG | PCSK9 | 1466 |
| R4321 | GUGUCCGGAGGCAGCCUGCG | PCSK9 | 1467 |
| R4322 | GCCACCAGGCGGCCAGUGUC | PCSK9 | 1468 |
| R4323 | CUGCUGCCAUGCCCCAGGGC | PCSK9 | 1469 |
| R4324 | CAGCCCUGGGGCAUGGCAGC | PCSK9 | 1470 |
| R4325 | CAUUCCAGCCCUGGGGCAUG | PCSK9 | 1471 |
| R4326 | GCAUUCCAGCCCUGGGGCAU | PCSK9 | 1472 |
| R4327 | UGCAUUCCAGCCCUGGGGCA | PCSK9 | 1473 |
| R4328 | AUUUUGCAUUCCAGCCCUGG | PCSK9 | 1474 |
| R4329 | CAUCCAGUCAGGGUCCAUCC | PCSK9 | 1475 |
| R4330 | UCCACGCUGUAGGCUCCCAG | PCSK9 | 1476 |
| R4331 | CCACACACAGGUUGUCCACG | PCSK9 | 1477 |
| R4332 | UCCACUGGUCCUGUCUGCUC | PCSK9 | 1478 |
| R4333 | CUGAAGGCCGGCUCCGGCAG | PCSK9 | 1479 |

TABLE 24
CasΦ.12 gRNAs targeting mouse PCSK9

| | Repeat + spacer sequence RNA | SEQ ID |
|---|---|---|
| Name | Sequence (5' --> 3') | NO |
| R4238_CasPhi12 | CUUUCAAGACUAAUAGAUUGCUCCUUACGAGGAGA | 1480 |
| | CCCGCUGUUGCCGCCGCUGCU | |
| R4239_CasPhi12 | CUUUCAAGACUAAUAGAUUGCUCCUUACGAGGAGA | 1481 |
| | CCCGCCGCUGCUGCUGCUGUU | |
| R4240_CasPhi12 | CUUUCAAGACUAAUAGAUUGCUCCUUACGAGGAGA | 1482 |
| | CCUGCUACUGUGCCCCACCGG | |
| R4241_CasPhi12 | CUUUCAAGACUAAUAGAUUGCUCCUUACGAGGAGA | 1483 |
| | CAUAAUCUCCAUCCUCGUCCU | |
| R4242_CasPhi12 | CUUUCAAGACUAAUAGAUUGCUCCUUACGAGGAGA | 1484 |
| | CUGAAGAGCUGAUGCUCGCCC | |
| R4243_CasPhi12 | CUUUCAAGACUAAUAGAUUGCUCCUUACGAGGAGA | 1485 |
| | CGAGCAACGGCGGAAGGUGGC | |
| R4244_CasPhi12 | CUUUCAAGACUAAUAGAUUGCUCCUUACGAGGAGA | 1486 |
| | CCUGGCAGCCUCCAGGCCUCC | |
| R4245_CasPhi12 | CUUUCAAGACUAAUAGAUUGCUCCUUACGAGGAGA | 1487 |
| | CUGGUGCUGAUGGAGGAGACC | |
| R4246_CasPhi12 | CUUUCAAGACUAAUAGAUUGCUCCUUACGAGGAGA | 1488 |
| | CAAUCUGUAGCCUCUGGGUCU | |
| R4247_CasPhi12 | CUUUCAAGACUAAUAGAUUGCUCCUUACGAGGAGA | 1489 |
| | CUUCAAUCUGUAGCCUCUGGG | |
| R4248_CasPhi12 | CUUUCAAGACUAAUAGAUUGCUCCUUACGAGGAGA | 1490 |
| | CGUUCAAUCUGUAGCCUCUGG | |
| R4249_CasPhi12 | CUUUCAAGACUAAUAGAUUGCUCCUUACGAGGAGA | 1491 |
| | CAACAAACUGCCCACCGCCUG | |
| R4250_CasPhi12 | CUUUCAAGACUAAUAGAUUGCUCCUUACGAGGAGA | 1492 |
| | CAUGACAUAGCCCCGGCGGGC | |
| R4251_CasPhi12 | CUUUCAAGACUAAUAGAUUGCUCCUUACGAGGAGA | 1493 |
| | CUACAUAUCUUUUAUGACCUC | |
| R4252_CasPhi12 | CUUUCAAGACUAAUAGAUUGCUCCUUACGAGGAGA | 1494 |
| | CUAUGACCUCUUCCCUGGCUU | |
| R4253_CasPhi12 | CUUUCAAGACUAAUAGAUUGCUCCUUACGAGGAGA | 1495 |
| | CAUGACCUCUUCCCUGGCUUC | |
| R4254_CasPhi12 | CUUUCAAGACUAAUAGAUUGCUCCUUACGAGGAGA | 1496 |
| | CUGACCUCUUCCCUGGCUUCU | |
| R4255_CasPhi12 | CUUUCAAGACUAAUAGAUUGCUCCUUACGAGGAGA | 1497 |
| | CACCAAGAAGCCAGGGAAGAG | |
| R4256_CasPhi12 | CUUUCAAGACUAAUAGAUUGCUCCUUACGAGGAGA | 1498 |
| | CCCUGGCUUCUUGGUGAAGAU | |
| R4257_CasPhi12 | CUUUCAAGACUAAUAGAUUGCUCCUUACGAGGAGA | 1499 |
| | CUUGGUGAAGAUGAGCAGUGA | |
| R4258_CasPhi12 | CUUUCAAGACUAAUAGAUUGCUCCUUACGAGGAGA | 1500 |
| | CGUGAAGAUGAGCAGUGACCU | |
| R4259_CasPhi12 | CUUUCAAGACUAAUAGAUUGCUCCUUACGAGGAGA | 1501 |
| | CCCCCAUGUGGAGUACAUUGA | |
| R4260_CasPhi12 | CUUUCAAGACUAAUAGAUUGCUCCUUACGAGGAGA | 1502 |
| | CCUCAAUGUACUCCACAUGGG | |
| R4261_CasPhi12 | CUUUCAAGACUAAUAGAUUGCUCCUUACGAGGAGA | 1503 |
| | CAGGAAGACUCCUUUGUCUUC | |
| R4262_CasPhi12 | CUUUCAAGACUAAUAGAUUGCUCCUUACGAGGAGA | 1504 |
| | CGUCUUCGCCCAGAGCAUCCC | |
| R4263_CasPhi12 | CUUUCAAGACUAAUAGAUUGCUCCUUACGAGGAGA | 1505 |
| | CUCUUCGCCCAGAGCAUCCCA | |
| R4264_CasPhi12 | CUUUCAAGACUAAUAGAUUGCUCCUUACGAGGAGA | 1506 |
| | CGCCCAGAGCAUCCCAUGGAA | |
| R4265_CasPhi12 | CUUUCAAGACUAAUAGAUUGCUCCUUACGAGGAGA | 1507 |
| | CCAUGGGAUGCUCUGGGCGAA | |
| R4266_CasPhi12 | CUUUCAAGACUAAUAGAUUGCUCCUUACGAGGAGA | 1508 |
| | CGCUCCAGGUUCCAUGGGAUG | |
| R4267_CasPhi12 | CUUUCAAGACUAAUAGAUUGCUCCUUACGAGGAGA | 1509 |
| | CUCCCAGCAUGGCACCAGACA | |
| R4268_CasPhi12 | CUUUCAAGACUAAUAGAUUGCUCCUUACGAGGAGA | 1510 |
| | CCUCUGUCUGGUGCCAUGCUG | |
| R4269_CasPhi12 | CUUUCAAGACUAAUAGAUUGCUCCUUACGAGGAGA | 1511 |
| | CGAUACCAGCAUCCAGGGUGC | |
| R4270_CasPhi12 | CUUUCAAGACUAAUAGAUUGCUCCUUACGAGGAGA | 1512 |
| | CAGGGCAGGGUCACCAUCACC | |
| R4271_CasPhi12 | CUUUCAAGACUAAUAGAUUGCUCCUUACGAGGAGA | 1513 |
| | CAAGUCGGUGAUGGUGACCCU | |
| R4272_CasPhi12 | CUUUCAAGACUAAUAGAUUGCUCCUUACGAGGAGA | 1514 |
| | CAACAGCGUGCCGGAGGAGGA | |
| R4273_CasPhi12 | CUUUCAAGACUAAUAGAUUGCUCCUUACGAGGAGA | 1515 |
| | CGCCACACCAGCAUCCCGGCC | |
| R4274_CasPhi12 | CUUUCAAGACUAAUAGAUUGCUCCUUACGAGGAGA | 1516 |
| | CAGCACACGCAGGCUGUGCAG | |
| R4275_CasPhi12 | CUUUCAAGACUAAUAGAUUGCUCCUUACGAGGAGA | 1517 |
| | CACAGUUGAGCACACGCAGGC | |
| R4276_CasPhi12 | CUUUCAAGACUAAUAGAUUGCUCCUUACGAGGAGA | 1518 |
| | CCCUUGACAGUUGAGCACACG | |
| R4277_CasPhi12 | CUUUCAAGACUAAUAGAUUGCUCCUUACGAGGAGA | 1519 |
| | CGCUGACUCUUCCGAAUAAAC | |
| R4278_CasPhi12 | CUUUCAAGACUAAUAGAUUGCUCCUUACGAGGAGA | 1520 |
| | CAUUCGGAAGAGUCAGCUAAU | |
| R4279_CasPhi12 | CUUUCAAGACUAAUAGAUUGCUCCUUACGAGGAGA | 1521 |
| | CUUCGGAAGAGUCAGCUAAUC | |
| R4280_CasPhi12 | CUUUCAAGACUAAUAGAUUGCUCCUUACGAGGAGA | 1522 |
| | CGGAAGAGUCAGCUAAUCCAG | |
| R4281_CasPhi12 | CUUUCAAGACUAAUAGAUUGCUCCUUACGAGGAGA | 1523 |
| | CUGCUGCCCCUGGCCGGUGGG | |
| R4282_CasPhi12 | CUUUCAAGACUAAUAGAUUGCUCCUUACGAGGAGA | 1524 |
| | CAGGAUGCGGCUAUACCCACC | |
| R4283_CasPhi12 | CUUUCAAGACUAAUAGAUUGCUCCUUACGAGGAGA | 1525 |
| | CCCAGCUGCUGCAACCAGCAC | |
| R4284_CasPhi12 | CUUUCAAGACUAAUAGAUUGCUCCUUACGAGGAGA | 1526 |
| | CCAGCAGCUGGGAACUUCCGG | |
| R4285_CasPhi12 | CUUUCAAGACUAAUAGAUUGCUCCUUACGAGGAGA | 1527 |
| | CCGGGACGACGCCUGCCUCUA | |
| R4286_CasPhi12 | CUUUCAAGACUAAUAGAUUGCUCCUUACGAGGAGA | 1528 |
| | CGUGGCCCCGACUGUGAUGAC | |
| R4287_CasPhi12 | CUUUCAAGACUAAUAGAUUGCUCCUUACGAGGAGA | 1529 |
| | CCCUUGGGGACUUUGGGGACU | |
| R4288_CasPhi12 | CUUUCAAGACUAAUAGAUUGCUCCUUACGAGGAGA | 1530 |
| | CGUCCCCAAAGUCCCCAAGGU | |
| R4289_CasPhi12 | CUUUCAAGACUAAUAGAUUGCUCCUUACGAGGAGA | 1531 |
| | CGGGACUUUGGGGACUAAUUU | |
| R4290_CasPhi12 | CUUUCAAGACUAAUAGAUUGCUCCUUACGAGGAGA | 1532 |
| | CGGGGACUAAUUUUGGACGCU | |
| R4291_CasPhi12 | CUUUCAAGACUAAUAGAUUGCUCCUUACGAGGAGA | 1533 |
| | CGGGACUAAUUUUGGACGCUG | |
| R4292_CasPhi12 | CUUUCAAGACUAAUAGAUUGCUCCUUACGAGGAGA | 1534 |
| | CUGGACGCUGUGUGGAUCUCU | |
| R4293_CasPhi12 | CUUUCAAGACUAAUAGAUUGCUCCUUACGAGGAGA | 1535 |
| | CGGACGCUGUGUGGAUCUCUU | |
| R4294_CasPhi12 | CUUUCAAGACUAAUAGAUUGCUCCUUACGAGGAGA | 1536 |
| | CGACGCUGUGUGGAUCUCUUU | |
| R4295_CasPhi12 | CUUUCAAGACUAAUAGAUUGCUCCUUACGAGGAGA | 1537 |
| | CCCGGGGGCAAAGAGAUCCAC | |
| R4296_CasPhi12 | CUUUCAAGACUAAUAGAUUGCUCCUUACGAGGAGA | 1538 |
| | CGCCCCCGGGAAGGACAUCAU | |
| R4297_CasPhi12 | CUUUCAAGACUAAUAGAUUGCUCCUUACGAGGAGA | 1539 |
| | CCCCCCGGGAAGGACAUCAUC | |
| R4298_CasPhi12 | CUUUCAAGACUAAUAGAUUGCUCCUUACGAGGAGA | 1540 |
| | CAUGUCACAGAGUGGGACCUC | |
| R4299_CasPhi12 | CUUUCAAGACUAAUAGAUUGCUCCUUACGAGGAGA | 1541 |
| | CUGGCUCGGAUGCUGAGCCGG | |
| R4300_CasPhi12 | CUUUCAAGACUAAUAGAUUGCUCCUUACGAGGAGA | 1542 |
| | CCCCUGGCCGAGCUGCGGCAG | |
| R4301_CasPhi12 | CUUUCAAGACUAAUAGAUUGCUCCUUACGAGGAGA | 1543 |
| | CGUAGAGAAGUGGAUCAGCCU | |
| R4302_CasPhi12 | CUUUCAAGACUAAUAGAUUGCUCCUUACGAGGAGA | 1544 |
| | CGGUAGAGAAGUGGAUCAGCC | |
| R4303_CasPhi12 | CUUUCAAGACUAAUAGAUUGCUCCUUACGAGGAGA | 1545 |
| | CUCUACCAAAGACGUCAUCAA | |
| R4304_CasPhi12 | CUUUCAAGACUAAUAGAUUGCUCCUUACGAGGAGA | 1546 |
| | CAUGACGUCUUUGGUAGAGAA | |
| R4305_CasPhi12 | CUUUCAAGACUAAUAGAUUGCUCCUUACGAGGAGA | 1547 |
| | CCCUGAGGACCAGCAGGUGCU | |
| R4306_CasPhi12 | CUUUCAAGACUAAUAGAUUGCUCCUUACGAGGAGA | 1548 |
| | CGGGGUCAGCACCUGCUGGUC | |
| R4307_CasPhi12 | CUUUCAAGACUAAUAGAUUGCUCCUUACGAGGAGA | 1549 |
| | CGAGUGGGCCCCGAGUGUGCC | |
| R4308_CasPhi12 | CUUUCAAGACUAAUAGAUUGCUCCUUACGAGGAGA | 1550 |
| | CUGGGGCACAGCGGGCUGUAG | |
| R4309_CasPhi12 | CUUUCAAGACUAAUAGAUUGCUCCUUACGAGGAGA | 1551 |
| | CUCCAGGAGCGGGAGGCGUCG | |
| R4310_CasPhi12 | CUUUCAAGACUAAUAGAUUGCUCCUUACGAGGAGA | 1552 |
| | CCAGACCUGCUGGCCUCCUAU | |
| R4311_CasPhi12 | CUUUCAAGACUAAUAGAUUGCUCCUUACGAGGAGA | 1553 |
| | CAGGGCCUUGCAGACCUGCUG | |
| R4312_CasPhi12 | CUUUCAAGACUAAUAGAUUGCUCCUUACGAGGAGA | 1554 |
| | CGGGGGUGAGGGUGUCUAUGC | |
| R4313_CasPhi12 | CUUUCAAGACUAAUAGAUUGCUCCUUACGAGGAGA | 1555 |
| | CGGGGUGAGGGUGUCUAUGCC | |
| R4314_CasPhi12 | CUUUCAAGACUAAUAGAUUGCUCCUUACGAGGAGA | 1556 |
| | CGCACGGGGAACCAGGCAGCA | |
| R4315_CasPhi12 | CUUUCAAGACUAAUAGAUUGCUCCUUACGAGGAGA | 1557 |
| | CCCCGUGCCAACUGCAGCAUC | |
| R4316_CasPhi12 | CUUUCAAGACUAAUAGAUUGCUCCUUACGAGGAGA | 1558 |
| | CUGGAUGCUGCAGUUGGCACG | |
| R4317_CasPhi12 | CUUUCAAGACUAAUAGAUUGCUCCUUACGAGGAGA | 1559 |
| | CUGGUGGCAGUGGACAUGGGU | |
| R4318_CasPhi12 | CUUUCAAGACUAAUAGAUUGCUCCUUACGAGGAGA | 1560 |
| | CCACUUCCCAAUGGAAGCUGC | |
| R4319_CasPhi12 | CUUUCAAGACUAAUAGAUUGCUCCUUACGAGGAGA | 1561 |
| | CCAUUGGGAAGUGGAAGACCU | |
| R4320_CasPhi12 | CUUUCAAGACUAAUAGAUUGCUCCUUACGAGGAGA | 1562 |
| | CGGAAGUGGAAGACCUUAGUG | |
| R4321_CasPhi12 | CUUUCAAGACUAAUAGAUUGCUCCUUACGAGGAGA | 1563 |
| | CGUGUCCGGAGGCAGCCUGCG | |
| R4322_CasPhi12 | CUUUCAAGACUAAUAGAUUGCUCCUUACGAGGAGA | 1564 |
| | CGCCACCAGGCGGCCAGUGUC | |
| R4323_CasPhi12 | CUUUCAAGACUAAUAGAUUGCUCCUUACGAGGAGA | 1565 |
| | CCUGCUGCCAUGCCCCAGGGC | |
| R4324_CasPhi12 | CUUUCAAGACUAAUAGAUUGCUCCUUACGAGGAGA | 1566 |
| | CCAGCCCUGGGGCAUGGCAGC | |
| R4325_CasPhi12 | CUUUCAAGACUAAUAGAUUGCUCCUUACGAGGAGA | 1567 |
| | CCAUUCCAGCCCUGGGGCAUG | |
| R4326_CasPhi12 | CUUUCAAGACUAAUAGAUUGCUCCUUACGAGGAGA | 1568 |
| | CGCAUUCCAGCCCUGGGGCAU | |
| R4327_CasPhi12 | CUUUCAAGACUAAUAGAUUGCUCCUUACGAGGAGA | 1569 |
| | CUGCAUUCCAGCCCUGGGGCA | |
| R4328_CasPhi12 | CUUUCAAGACUAAUAGAUUGCUCCUUACGAGGAGA | 1570 |
| | CAUUUUGCAUUCCAGCCCUGG | |
| R4329_CasPhi12 | CUUUCAAGACUAAUAGAUUGCUCCUUACGAGGAGA | 1571 |
| | CCAUCCAGUCAGGGUCCAUCC | |
| R4330_CasPhi12 | CUUUCAAGACUAAUAGAUUGCUCCUUACGAGGAGA | 1572 |
| | CUCCACGCUGUAGGCUCCCAG | |
| R4331_CasPhi12 | CUUUCAAGACUAAUAGAUUGCUCCUUACGAGGAGA | 1573 |
| | CCCACACACAGGUUGUCCACG | |
| R4332_CasPhi12 | CUUUCAAGACUAAUAGAUUGCUCCUUACGAGGAGA | 1574 |
| | CUCCACUGGUCCUGUCUGCUC | |
| R4333_CasPhi12 | CUUUCAAGACUAAUAGAUUGCUCCUUACGAGGAGA | 1575 |
| | CCUGAAGGCCGGCUCCGGCAG | |

### Example 11. Optimization of Lipid Nanoparticle Delivery of CasΦ

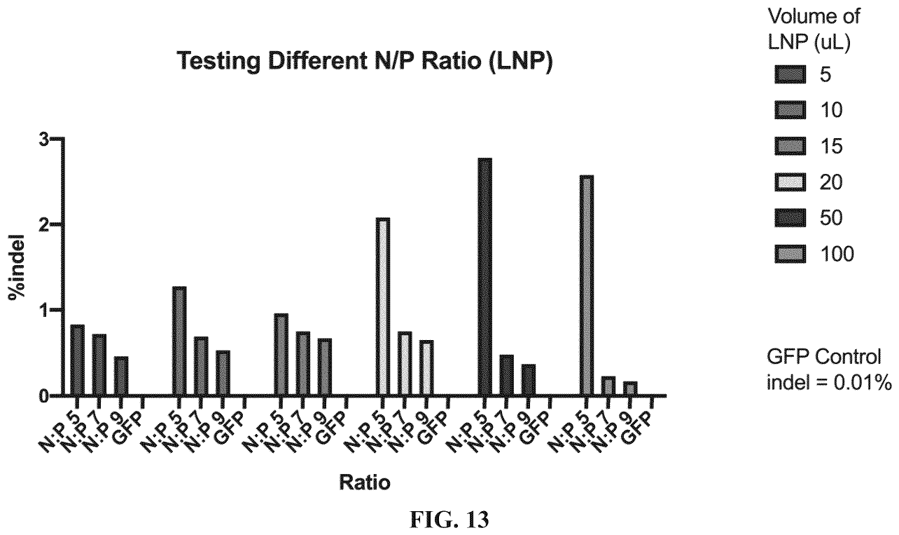

This example describes the optimization of lipid nanoparticle (LNP) delivery of CasΦ mRNA and gRNA. In this study, the encapsulation efficiency of LNPs was optimized by testing different amine group to phosphate group ratio (N/P) of LNPs containing CasΦ mRNA and gRNA. An LNP kit from Precision Nanosystems (GenVoy-ILM™) was used to generate LNPs with different N/P ratios. LNPs were then dropped into HEK293T cells. Genomic DNA was extracted and the frequency of indel mutations was determined using NGS. The gRNA used in this study was R2470 with 2′O-methyl on the first three 5′ and last three 3′ nucleotides and phosphorothioate bonds in between the first three 5′ nucleotides and in between the last two 3′ nucleotides. The mRNA was generated using T7 messenger mRNA IVT kit. As shown in FIG. 13, indel mutations were detected following the use of a range of N/P ratios.

LNPs are one of the most clinically advanced non-viral delivery systems for gene therapy. LNPs have many properties that make them ideal candidates for delivery of nucleic acids, including ease of manufacture, low cytotoxicity and immunogenicity, high effiency of nucleic acid encapsulation and cell transfection, multidosing capabilities and flexibility of design (Kulkami et al., (2018) Nucleic Acid Therapeutics).

### Example 12. Genome Editing with CasΦ Polypeptides Mediates Efficient Editing of CIITA Locus

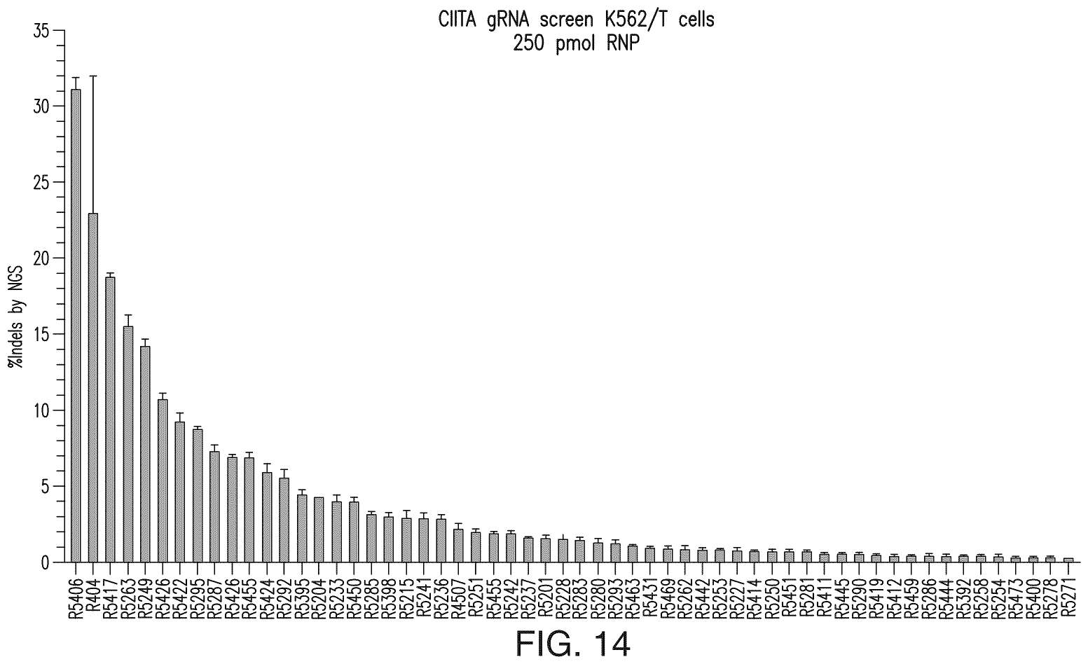

This example demonstrates CasΦ-mediated genome editing of the CIITA locus. In this study, RNP complexes were formed using CasΦ polypeptides and gRNAs targeting CIITA (sequences shown in TABLE 7 and TABLE 8). K562 cells were nucleofected with RNP complexes (250 μmol) using Lonza nucleofection protocols. Cells were harvested after 48 hours, genomic DNA was isolated and the frequency of indel mutations was evaluated using NGS analysis (MiSeq, Illumina). As shown in FIG. 14, effective genome editing of the CIITA locus was achieved using CasΦ RNP complexes.

### Example 13. PAM Screening for Effector Proteins

Effector proteins and guide RNA combinations represented in TABLE 27 were screened by in vitro enrichment (IVE) for PAM recognition. TABLE 27 shows the components of each effector protein-guide RNA complex assayed for PAM recognition. The amino acid sequences of the effector protein names in the second column of the TABLE are shown in TABLE 1 herein. The nucleotide sequences of the guide components in the third through sixth columns of the TABLE are shown in TABLE 25 and TABLE 26 herein. For example, as shown in TABLE 25, an effector protein comprising an amino acid sequence of SEQ ID NO: 1 complexed with a guide comprising a crRNA of SEQ ID NO: 347 and a tracrRNA of SEQ ID NO: 385 was screened for PAM recognition. Briefly, effector proteins were complexed with corresponding guide RNAs for 15 minutes at 37° C. The complexes were added to an IVE reaction mix. PAM screening reactions used 10 μl of RNP in 100 μl reactions with 1,000 ng of a 5′ PAM library in 1× Cutsmart buffer and were carried out for 15 minutes at 25° C., 45 minutes at 37° C. and 15 minutes at 45° C. Reactions were terminated with 1 μl of proteinase K and 5 μl of 500 mM EDTA for 30 minutes at 37° C. Next generation sequencing was performed on cut sequences to identify enriched PAMs. As shown in TABLE 27, cis cleavages were observed with RNP complexes comprising effector proteins and corresponding guide RNAs.

TABLE 25
Exemplary crRNA and tracRNA for CasM Effector Proteins

| Comp. | | | |
|---|---|---|---|
| No. | Protein | crRNA (repeat) | tracrRNA |
| 1 | CasM.298706 | CGUUGCAGCUCGCAC | GGGGCGUCUUCCCGUCCCUAAA |
| | (SEQ ID NO: | GUUGGCACUGGUUGA | UCGAGAUAGCAGCCAUUUUUCU |
| | 1) | AGGUAUUAAAUACUC | UCAUUUUUGAAGACGGUCUUGC |
| | | GUAUUGCU (SEQ ID | ACUCGAAAAGGUCAAG (SEQ ID |
| | | NO: 347) | NO: 385) |
| 2 | CasM.280604 | GUUGCAACUCACGCG | GGGGCGACUUCCCGCCCCAAAA |
| | (SEQ ID NO: | CGUAUGUGGCUUGAA | UCGAGAAAGUGACUGUCAGACU |
| | 2) | GGUAUUAAAUACUCG | UUGCUAUGCAAAGCAAGUAAUA |
| | | UAUUGCU (SEQ ID NO: | CACUCGAGAAGGUAAAGA (SEQ |
| | | 348) | ID NO: 386) |
| 3 | CasM.281060 | GUUGCAAUUCAUAUC | AGGGCGACUUCCCGUCCUAAAA |
| | (SEQ ID NO: | UCCGGGUGGAUUGAA | UCGAGAAAGUGACAAUUCAGUC |
| | 3) | GGUAUUAAAUACUCG | UCGCAUUUCGAGCAUUGUAAUA |
| | | UAUUGCU (SEQ ID NO: | CACUCGAAAAGGUUAAG (SEQ |
| | | 349) | ID NO: 387) |
| 4 | CasM.284933 | GUUGCAGCGUGCGCG | GGGGCGACUUCCCGUCCCAAAA |
| | (SEQ ID NO: | AGCGUGUGGCUUGAA | UCGAGAAAGUGGUCGUAAGUCU |
| | 4) | GGUAUUAAAUACUCG | CGAUCGGAUCGAAGCAGACAAU |
| | | UAUUGCU (SEQ ID NO: | ACACUCGAAAAGGUUAAGU |
| | | 350) | (SEQ ID NO: 388) |
| 5 | CasM.287908 | GUUGCAACUCGCACG | GGGGCGACUUCCCGUCCCUAAA |
| | (SEQ ID NO: | UGAAUGCGACUUGAA | UCGAGAAAGUGGCGGUAAGACU |
| | 5) | GGUAUUAAAUACUCG | UCGGUCUUCGAAGCGCGCAAUA |
| | | UAUUGCU (SEQ ID NO: | CACUCGAAAAGGUUAA (SEQ ID |
| | | 351) | NO: 389) |
| 6 | CasM.288518 | GAUGCAACUCGUGUG | GGGGCGACUUCCCGUCCCAAAA |
| | (SEQ ID NO: | UAUGUGCGAGUUGAA | UCGAGAAAGUGACAGUAAUUCU |
| | 6) | GGUAUUAAAUACUCG | UUGUUUUACAGAGGUUGUAAU |
| | | UAUUGCU (SEQ ID NO: | ACACUCGAUAAGGUUAAG (SEQ |
| | | 352) | ID NO: 390) |
| 7 | CasM.293891 | GACGCAACUCGCGCG | GGGGCGACCUCCCGUCCCAAAA |
| | (SEQ ID NO: | CGGGCAUGUAUUGAG | UCGAGAAAGUGGCCGUCAGACU |
| | 7) | GGUAUUAAAUACUCG | UCUCGCUGAGAAGCACGCAAUA |
| | | UAUUGCU (SEQ ID NO: | CACUCGAAAAGGUAAAG (SEQ |
| | | 353) | ID NO: 391) |
| 8 | CasM.294270 | GAUGCAUCUGACACA | AGGGCGACUUCCCGUCCUGAAA |
| | (SEQ ID NO: | GCUGGGUGAGUUGAA | UCGAGAAAGUGACAAGGAAAGC |
| | 8) | GGUAUUAAAUACUCG | GCAAUUUUGCGCCGUUGUAAUA |
| | | UAUUGCU (SEQ ID NO: | CACUCGAGAAGGUCAAG (SEQ |
| | | 354) | ID NO: 392) |
| 9 | CasM.294491 | GUUGCAACACAUGUA | AGGGCGACUUCCCGUCCUAAAA |
| | (SEQ ID NO: | UGUGGGUGAGUUGAA | UCGAGAUAGUGACAAGUCAGUC |
| | 9) | GGUAUUAAAUACUCG | UCUUAUGAGGAGCAUUGUAAUA |
| | | UAUUGCU (SEQ ID NO: | CACUCGAGAAGGUCAAG (SEQ |
| | | 355) | ID NO: 393) |
| 10 | CasM.295047 | GUUGCAGCGUGCGCG | GGGGCGACUUCCCGUCCCAAAA |
| | (SEQ ID NO: | AGCGUGUGGCUUGAA | UCGAGAAAGUGGUCGUAAGUCU |
| | 10) | GGUAUUAAAUACUCG | CGAUCGGAUCGAAGCAGACAAU |
| | | UAUUGCU (SEQ ID NO: | ACACUCGAAAAGGUUAAGU |
| | | 350) | (SEQ ID NO: 388) |
| 11 | CasM.299588 | GUUGCAAUUUGUAUA | AGGGCGACUUCACGUCCUCAAA |
| | (SEQ ID NO: | CGAGUGUGACUUGAA | UCGAGAAAGUGAGCGUAAGACU |
| | 11) | GGUAUUAAAUACUCG | UGGCUUCUGUCAAGCGGUUAAU |
| | | UAUUGCU (SEQ ID NO: | ACACUCGAGAAGGUUAA (SEQ |
| | | 356) | ID NO: 394) |
| 12 | CasM.277328 | GCUGCAACACGCGCG | GGGGCGACUUCCCGUCCCGAAA |
| | (SEQ ID NO: | GGUACGCGGGUUGAA | UCGAGAAAGUGACCGUCAGACU |
| | 12) | GGUAUUAAAUACUCG | CUGCUUUGCAGAGCAGGUAAUA |
| | | UAUUGCU (SEQ ID NO: | CACUCGAGAAGGUAAAG (SEQ |
| | | 357) | ID NO: 395) |
| 13 | CasM.297894 | GUUGCAACUCGCACG | GGGGCGUCUUCCCGUCCCUAAA |
| | (SEQ ID NO: | UUGGCACUGAUUGAA | UCGAGAUAGCAGCCAUUUUUCU |
| | 13) | GGUAUUAAAUACUCG | UCAUUUUUUGAAGACGGUCUUG |
| | | UAUUGCU (SEQ ID NO: | CACUCGAAAAGGUCAAG (SEQ |
| | | 358) | ID NO: 396) |
| 14 | CasM.291449 | GCUGUAGCCCUGCUC | CACGCTAGCTGAAAAGCAACCG |
| | (SEQ ID NO: | AAAUUGUAGGGCGCA | CGTACACGCGGACGAACGGCCG |
| | 14) | UGCAGGUAUUAAAUA | ACCTGCTCGGCCTGAAGGTTGAG |
| | | CUCGUAUUGCU (SEQ | AAGGTTATGTATAAGAGGAGAA |
| | | ID NO: 359) | AATCCCCCTTCATAATCGCTCAC |
| | | | CAAGCTCCCAATTTACATATTTT |
| | | | (SEQ ID NO: 397) |
| 15 | CasM.291449 | GCUGUAGCCCUGCUC | CGGCCGACCUGCUCGGCCUGAA |
| | (SEQ ID NO: | AAAUUGUAGGGCGCA | GGUUGAGAAGGUUAUGUAUAA |
| | 14) | UGCAGGUAUUAAAUA | GAGGAGAAAAUCCCCCUUCAUA |
| | | CUCGUAUUGCU (SEQ | AUCGCUCACCAAGCUCCCAAUU |
| | | ID NO: 359) | UACAUAUUUU (SEQ ID NO: 398) |
| 16 | CasM.297599 | GUUGUAGUCGACCUG | TATTGCGCTAGCCATAATGGCAA |
| | (SEQ ID NO: | AAUCUGUGGGGUGCU | TCGCGTACAGGCAACTGAAGGC |
| | 15) | UACAGGUAUUAAAUA | CGACCTGTACGGCCTTAAGGTTG |
| | | CUCGUAUUGCU (SEQ | AGAAGGCACATGTAAGTGGAAA |
| | | ID NO: 360) | AATGCTTTCCCGTTGTGTTCGCT |
| | | | CACCAAGCACACACGTTTTTTT |
| | | | (SEQ ID NO: 399) |
| 17 | CasM.297599 | GUUGUAGUCGACCUG | GAAGGCCGACCUGUACGGCCUU |
| | (SEQ ID NO: | AAUCUGUGGGGUGCU | AAGGUUGAGAAGGCACAUGUAA |
| | 15) | UACAGGUAUUAAAUA | GUGGAAAAAUGCUUUCCCGUUG |
| | | CUCGUAUUGCU (SEQ | UGUUCGCUCACCAAGCACACAC |
| | | ID NO: 360) | GUUUUUUU (SEQ ID NO: 400) |
| 18 | CasM.286588 | GGUGUAUGUAACCGC | AGGTCGCCGTTTACGTTGCGTCA |
| | (SEQ ID NO: | AAUUUGAAGGGUGCA | CAAGGGCGCGCGGGCGACCGAA |
| | 16) | UACAGGUAUUAAAUA | GGCCGATCTGTACGGCCTGCAGG |
| | | CUCGUAUUGCU (SEQ | TTGAGAAGGCACATATTAGAGG |
| | | ID NO: 361) | AAAATTGCTTCCCTTTGTGTTCG |
| | | | CTCACCGAGTATTCCTTGTTTTTT |
| | | | (SEQ ID NO: 401) |
| 19 | CasM.286588 | GGUGUAUGUAACCGC | AUCUGUACGGCCUGCAGGUUGA |
| | (SEQ ID NO: | AAUUUGAAGGGUGCA | GAAGGCACAUAUUAGAGGAAAA |
| | 16) | UACAGGUAUUAAAUA | UUGCUUCCCUUUGUGUUCGCUC |
| | | CUCGUAUUGCU (SEQ | ACCGAGUAUUCCUUGUUUUUU |
| | | ID NO: 361) | (SEQ ID NO: 402) |
| 20 | CasM.286910 | GUUGGAAUCGACCUU | CAATGTTTCGCTAACCTTTAAGG |
| | (SEQ ID NO: | AAUUUGAGGUGUGCU | TAATCGCGGGCAGGCGACTGAA |
| | 17) | UACAGGUAUUAAAUA | GGCCGACCTGTACGGCCTTAAGG |
| | | CUCGUAUUGCU (SEQ | CTGAGAAGGCACATGTAAGTGG |
| | | ID NO: 362) | AAAAATGCTTTCCCGTTGTGTTC |
| | | | GCTCACCAAGCACATTTGTTTTT |
| | | | TT (SEQ ID NO: 403) |
| 21 | CasM.286910 | GUUGGAAUCGACCUU | GAAGGCCGACCUGUACGGCCUU |
| | (SEQ ID NO: | AAUUUGAGGUGUGCU | AAGGCUGAGAAGGCACAUGUAA |
| | 17) | UACAGGUAUUAAAUA | GUGGAAAAAUGCUUUCCCGUUG |
| | | CUCGUAUUGCU (SEQ | UGUUCGCUCACCAAGCACAUUU |
| | | ID NO: 362) | GUUUUUUU (SEQ ID NO: 404) |
| 22 | CasM.292335 | GCUGAAAGAGCAGAG | AGGCCGTTATCAACGTTTCGCGG |
| | (SEQ ID NO: | AAUUUGUUGUGUGCA | AAGAGCGGACGAACGGCTGAAG |
| | 18) | UACAGGUAUUAAAUA | GCCGACCTGTACGGCCTAAAGGT |
| | | CUCGUAUUGCU (SEQ | TGAGAAGGCACATGTAAGAGGA |
| | | ID NO: 363) | AAATCGCTTCCCTTTGTGTTCGC |
| | | | TCACCGGGTACACGCGTTTTTTT |
| | | | (SEQ ID NO: 405) |
| 23 | CasM.292335 | GCUGAAAGAGCAGAG | AGGCCGACCUGUACGGCCUAAA |
| | (SEQ ID NO: | AAUUUGUUGUGUGCA | GGUUGAGAAGGCACAUGUAAGA |
| | 18) | UACAGGUAUUAAAUA | GGAAAAUCGCUUCCCUUUGUGU |
| | | CUCGUAUUGCU (SEQ | UCGCUCACCGGGUACACGCGUU |
| | | ID NO: 363) | UUUUU (SEQ ID NO: 406) |
| 24 | CasM.293576 | GUUGGAGUCGGCUUG | TCGTAAATGTTGCGCTAGCCATA |
| | (SEQ ID NO: | AAUCUGCGGGGUGCU | ATGGCAATCGCGTACAGGCAAC |
| | 19) | UACAGGUAUUAAAUA | TGAAGGCCGACCTGTACGGCCTT |
| | | CUCGUAUUGCU (SEQ | AAGGTTGAGAAGGCACATGTCA |
| | | ID NO: 364) | GTGGAAAAATGCTTTCCCTTTGT |
| | | | GTTCGCTCACCAAGCACACGCGG |
| | | | TTTTTT (SEQ ID NO: 407) |
| 25 | CasM.293576 | GUUGGAGUCGGCUUG | AAGGCCGACCUGUACGGCCUUA |
| | ((SEQ ID | AAUCUGCGGGGUGCU | AGGUUGAGAAGGCACAUGUCAG |
| | NO: 19) | UACAGGUAUUAAAUA | UGGAAAAAUGCUUUCCCUUUGU |
| | | CUCGUAUUGCU (SEQ | GUUCGCUCACCAAGCACACGCG |
| | | ID NO: 364) | GUUUUUU (SEQ ID NO: 408) |
| 26 | CasM.294537 | GUUGGAAUCGACCUU | AATGTTTCGCTAACCTTTAAGGT |
| | (SEQ ID NO: | AAUUUGAGGUGUGCU | AATCGCGGGCAGGCGACTGAAG |
| | 20) | UACAGGUAUUAAAUA | GCCGACCTGTACGGCCTTAAGGC |
| | | CUCGUAUUGCU (SEQ | TGAGAAGGCACATGTAAGTGGA |
| | | ID NO: 362) | AAAATGCTTTCCCGTTGTGTTCG |
| | | | CTCACCAAGCACATTTGTTTTTTT |
| | | | (SEQ ID NO: 409) |
| 27 | CasM.294537 | GUUGGAAUCGACCUU | AAGGCCGACCUGUACGGCCUUA |
| | (SEQ ID NO: | AAUUUGAGGUGUGCU | AGGCUGAGAAGGCACAUGUAAG |
| | 20) | UACAGGUAUUAAAUA | UGGAAAAAUGCUUUCCCGUUGU |
| | | CUCGUAUUGCU (SEQ | GUUCGCUCACCAAGCACAUUUG |
| | | ID NO: 362) | UUUUUUU (SEQ ID NO: 410) |
| 28 | CasM.298538 | GUUGUAAGAGACCCG | GGTCGTTGTAAAACGTAACGCTA |
| | (SEQ ID NO: | AAUUUUAGCUGUGUA | GCCTTATGGCAATCGCGAACGA |
| | 21) | UACAGGUAUUAAAUA | ACGACTGAAGGCCGACCTGTAC |
| | | CUCGUAUUGCU (SEQ | GGCCTGAAGGATGAGAAGGCAC |
| | | ID NO: 365) | ATATTAGAGGAAAAAAATGGTT |
| | | | CCCTTTGTGACCGCTCACCAAAC |
| | | | ACATGTTTATTTTT (SEQ ID NO: |
| | | | 411) |
| 29 | CasM.298538 | GUUGUAAGAGACCCG | AAGGCCGACCUGUACGGCCUGA |
| | (SEQ ID NO: | AAUUUUAGCUGUGUA | AGGAUGAGAAGGCACAUAUUAG |
| | 21) | UACAGGUAUUAAAUA | AGGAAAAAAAUGGUUCCCUUUG |
| | | CUCGUAUUGCU (SEQ | UGACCGCUCACCAAACACAUGU |
| | | ID NO: 365) | UUAUUUUU (SEQ ID NO: 412) |
| 30 | CasM.19924 | GUUGUGAAUGCAGGC | AUGAAUAGGAUUCGUCCUAUGG |
| | (SEQ ID NO: | AUUUUUGAUGGUAAA | GGCAGUUGGUUGCCCUUAGCCU |
| | 22) | UCCAACUAUUAAAUA | GAGGCAUUUAUUGCACUCGGGA |
| | | CUCGUAUUGCU (SEQ | AGUACCAUUUCUCA (SEQ ID |
| | | ID NO: 366) | NO:413) |
| 32 | CasM.19952 | ACUGUCAGACAAUGC | AUGAAUAGGAUUCGUCCUAUGG |
| | (SEQ ID NO: | AAAAUGUGUGGUACA | GGCAGUUGGUUGCCCUUAGCCU |
| | 23) | UCCAACUAUUAAAUA | GAGGCAUUUAUUGCACUCGGGA |
| | | CUCGUAUUGCU (SEQ | AGUACCAUUUCUCA (SEQ ID NO: |
| | | ID NO: 367) | 413) |
| 34 | CasM.274559 | GCUGUCAGUAGUAGU | AUGAAUAGGAUUUAUCCUAUGG |
| | (SEQ ID NO: | AAAAAUGGGGGUACA | GGCAGUUGGUUGCCCUUAGCCU |
| | 24) | UCCAACUAUUAAAUA | GAGGCAUUUAAUGCACUCGGGA |
| | | CUCGUAUUGCU (SEQ | AGUACCUUUUCUCA (SEQ ID NO: |
| | | ID NO: 368) | 414) |
| 36 | CasM.286251 | ACUGUCAGUACAUGC | AAGAAUAGGAUUCAUCCUAUGG |
| | (SEQ ID NO: | AAAAAUGAGGGUACA | GGCAGUUGGUUGCCCUUAGCCU |
| | 25) | UCCAACUAUUAAAUA | GAGGAAUUUAAUUCACUCGGGA |
| | | CUCGUAUUGCU (SEQ | AGUACCUUUCUCAU (SEQ ID NO: |
| | | ID NO: 369) | 415) |
| 38 | CasM.288480 | ACUGUCAGACAAUGC | AUGAAUAGGAUUCGUCCUAUGG |
| | (SEQ ID NO: | AAAAUGAGUGGUACA | GGCAGUUGGUUGCCCUUAGCCU |
| | 26) | UCCAACUAUUAAAUA | GAGGCAUUUAUUGCACUCGGGA |
| | | CUCGUAUUGCU (SEQ | AGUACCAUUUCUCA (SEQ ID NO: |
| | | ID NO: 370) | 413) |
| 40 | CasM.288668 | GCUGUUAGAACAUAC | AUGGAUAGGAUUCGUCCUAUGG |
| | (SEQ ID NO: | AAAAUGAAAGGUACA | GGCAGUUGGGACCAUGUAAUGC |
| | 27) | UCCAACUAUUAAAUA | CCUUAGCCUGAGGAAUUCAUUU |
| | | CUCGUAUUGCU (SEQ | CACUCGGGAAGUAU (SEQ ID NO: |
| | | ID NO: 371) | 416) |
| 41 | CasM.289206 | GCUGCAUGUCAUGGC | AUGAAUAGGAUUUAUCCUAUGG |
| | (SEQ ID NO: | AAAAGGAAAGGUACA | GGCAGUUGGUUGCCCUUAGCCU |
| | 28) | UCCAACUAUUAAAUA | GAGGCAUUUAAUGCACUCGGGA |
| | | CUCGUAUUGCU (SEQ | AGUACCUUUUCUCA (SEQ ID NO: |
| | | ID NO: 372) | 414) |
| 43 | CasM.290598 | GCUGUCAGACACCUA | AUGAAUAGGAUUUAUCCUAUGG |
| | (SEQ ID NO: | AAAAAUGAGGGUACA | GGCAGUUGGUUGCCCUUAGCCU |
| | 29) | UCCAACUAUUAAAUA | GAGGCAUUUAAUGCACUCGGGA |
| | | CUCGUAUUGCU (SEQ | AGUACCUUUUCUCA (SEQ ID NO: |
| | | ID NO: 373) | 414) |
| 45 | CasM.290816 | GCUGUGAGUCACAGU | AUGAAUAGGAUUUAUCCUAUGG |
| | (SEQ ID NO: | AAAAAUGAAGGUAUA | GGCAGUUGGAUGCCCUUAGCCU |
| | 30) | UCCAACUAUUAAAUA | GAGGCAUUUAUUGCACUCGGGA |
| | | CUCGUAUUGCU (SEQ | AGUACCUUUUCUCA (SEQ ID NO: |
| | | ID NO: 374) | 417) |
| 47 | CasM.295071 | ACUGUCAGUACAUGC | AAGAAUAGGAUUCAUCCUAUGG |
| | (SEQ ID NO: | AAAAAUGAGGGUACA | GGCAGUUGGUUGCCCUUAGCCU |
| | 31) | UCCAACUAUUAAAUA | GAGGAAUUUAAUUCACUCGGGA |
| | | CUCGUAUUGCU (SEQ | AGUACCUUUCUCAU (SEQ ID NO: |
| | | ID NO: 369) | 415) |
| 49 | CasM.295231 | GCUGUGAGUCACAGU | AUGAAUAGGAUUUAUCCUAUGG |
| | (SEQ ID NO: | AAAAAUGAAGGUAUA | GGCAGUUGGAUGCCCUUAGCCU |
| | 32) | UCCAACUAUUAAAUA | GAGGCAUUUAUUGCACUCGGGA |
| | | CUCGUAUUGCU (SEQ | AGUACCUUUUCUCA (SEQ ID NO: |
| | | ID NO: 374) | 417) |
| 51 | CasM.292139 | GAUGUAUAUGCUAUG | UAUUUUCUAAUGGGGUUGUUG |
| | (SEQ ID NO: | AUUUUGUAUGGUACA | GAAAGAGCUUUUACUGAAAUUU |
| | 33) | UCCAACUAUUAAAUA | GUAAAGGUGCCCUGAACUUGAG |
| | | CUCGUAUUGCU (SEQ | AAUUGAAAAAUUACUCGAG |
| | | ID NO: 375) | (SEQ ID NO: 418) |
| 52 | CasM.292139 | GAUGUAUAUGCUAUG | AUGGGGUUGUUGGAAAGAGCU |
| | (SEQ ID NO: | AUUUUGUAUGGUACA | UUUACUGAAAUUUGUAAAGGU |
| | 33) | UCCAACUAUUAAAUA | GCCCUGAACUUGAGAAUUGAAA |
| | | CUCGUAUUGCU (SEQ | AAUUACUCGAG (SEQ ID NO: 419) |
| | | ID NO: 375) | |
| 54 | CasM.279423 | GCUGUCAGUAGUAGU | AUGAAUAGGAUUUAUCCUAUGG |
| | (SEQ ID NO: | AAAAAUGGGGGUACA | GGCAGUUGGUUGCCCUUAGCCU |
| | 34) | UCCAACUAUUAAAUA | GAGGCAUUUAAUGCACUCGGGA |
| | | CUCGUAUUGCU (SEQ | AGUACCUUUUCUCA (SEQ ID NO: |
| | | ID NO: 368) | 414) |
| 55 | CasM.20054 | GUUGAGCUCUGCAUU | TTCGGGCGGCTCGGCGTCCGTAA |
| | (SEQ ID NO: | ACGCAGAUGAAUGAC | ATCGAGAAAGAGCTTGTAATTCC |
| | 35) | GAGUAUUAAAUACUC | TGATTCTATCAGGTGAAGCAACA |
| | | GUAUUGCU (SEQ ID | CTCGGTAAGGTATAACAATACAC |
| | | NO: 376) | ATGTATAATCCGTGTATTTAAGT |
| | | | TCATTTT (SEQ ID NO: 420) |
| 56 | CasM.20054 | GUUGAGCUCUGCAUU | UUCGGGCGGCUCGGCGUCCGUA |
| | (SEQ ID NO: | ACGCAGAUGAAUGAC | AAUCGAGAAAGAGCUUGUAAUU |
| | 35) | GAGUAUUAAAUACUC | CCUGAUUCUAUCAGGUGAAGCA |
| | | GUAUUGCU (SEQ ID | ACACUCGGUAAGGUAUAAC |
| | | NO: 376) | (SEQ ID NO: 421) |
| 57 | CasM.282673 | GAUGCAACUUAGAUG | ATAAGGGCGGCTCAGCGTCCTA |
| | (SEQ ID NO: | CAUAUGUAAGUUGUG | AAGTCGAGAAAGTATGCGTAAA |
| | 36) | AGUAUUAAAUACUCG | CTTCTTTCATAGAATTGCAGATA |
| | | UAUUGCU (SEQ ID | CTCTCGGCAAGGTAAAAACCCTA |
| | | NO:377) | CAAATTTAATCCTTGTAGGCGAC |
| | | | TTATATTTGTGTATATTT (SEQ ID |
| | | | NO: 422) |
| 58 | CasM.282673 | GAUGCAACUUAGAUG | AUAAGGGCGGCUCAGCGUCCUA |
| | (SEQ ID NO: | CAUAUGUAAGUUGUG | AAGUCGAGAAAGUAUGCGUAAA |
| | 36) | AGUAUUAAAUACUCG | CUUCUUUCAUAGAAUUGCAGAU |
| | | UAUUGCU (SEQ ID NO: | ACUCUCGGCAAGGUAAAA (SEQ |
| | | 377) | ID NO: 423) |
| 59 | CasM.282952 | GUUGCAAUCUGCGUA | ATTCTTTCCTCGGAAAGTGGTAG |
| | (SEQ ID NO: | CAGGCGUAAGAUGUG | ATACTCTCGGTAAGGTAAACTGT |
| | 37) | AGUAUUAAAUACUCG | GTATGAACAGTTTGAAATCCTGC |
| | | UAUUGCU (SEQ ID NO: | ACATAAAATCCGTGCAGGCATCT |
| | | 378) | TATAGTTTTGTGCATCTTT (SEQ |
| | | | ID NO: 424) |
| 60 | CasM.282952 | GUUGCAAUCUGCGUA | AUUCUUUCCUCGGAAAGUGGUA |
| | (SEQ ID NO: | CAGGCGUAAGAUGUG | GAUACUCUCGGUAAGGUAAACU |
| | 37) | AGUAUUAAAUACUCG | GUGUAUGAACAGUUUGAAAUCC |
| | | UAUUGCU (SEQ ID NO: | UGCACAUAAAAUCCGUGCAGGC |
| | | 378) | AUC (SEQ ID NO: 425) |
| 61 | CasM.283262 | GAUCAUAUCUGCUUG | TTCGGGCGGCTCGGCGTCCGTAA |
| | (SEQ ID NO: | UAUGGGUAUGCUGCG | ACCGAGAAAGTATATGTAAGTCT |
| | 38) | AGUAUUAAAUACUCG | GAATTTATTCAGCGTTAGATACA |
| | | UAUUGCU (SEQ ID NO: | CTCGGTAAGGTTCAAACAATACA |
| | | 379) | TATTCAATCCATGTATTCAGTAT |
| | | | ATTTGTACATTTTT (SEQ ID NO: |
| | | | 426) |
| 62 | CasM.283262 | GAUCAUAUCUGCUUG | UUCGGGCGGCUCGGCGUCCGUA |
| | (SEQ ID NO: | UAUGGGUAUGCUGCG | AACCGAGAAAGUAUAUGUAAGU |
| | 38) | AGUAUUAAAUACUCG | CUGAAUUUAUUCAGCGUUAGAU |
| | | UAUUGCU (SEQ ID NO: | ACACUCGGUAAGGUUCAAAC |
| | | 379) | (SEQ ID NO: 427) |
| 63 | CasM.284833 | GUUGCAACUUACGCA | TTCAGGGCGACTCGGCGTCCTAA |
| | (SEQ ID NO: | UAGGUGUAAAAUACG | AATCGAGAAAGTGTACATAAAT |
| | 39) | AGUAUUAAAUACUCG | TTTTAACAAAATACGGTAAATAC |
| | | UAUUGCU (SEQ ID NO: | TCTCGGTAAGGTTTTAACGTGCA |
| | | 380) | CATAATAATCCGTGCAACAGGGT |
| | | | TACACTTTTGTGCAATTTT (SEQ |
| | | | ID NO: 428) |
| 64 | CasM.284833 | GUUGCAACUUACGCA | UUCAGGGCGACUCGGCGUCCUA |
| | (SEQ ID NO: | UAGGUGUAAAAUACG | AAAUCGAGAAAGUGUACAUAAA |
| | 39) | AGUAUUAAAUACUCG | UUUUUAACAAAAUACGGUAAAU |
| | | UAUUGCU (SEQ ID NO: | ACUCUCGGUAAGGUUUUAAC |
| | | 380) | (SEQ ID NO: 429) |
| 65 | CasM.287700 | GAUUAUAUCUGCUUG | UUCGGGCGGCUCGGCGUCCGUA |
| | ((SEQ ID | UAUGGGUAUACUGCG | AACCGAGAAAGUAUAUGUAAGU |
| | NO: 40) | AGUAUUAAAUACUCG | CUGAAUUUAUUCAGCGUUAGAU |
| | | UAUUGCU (SEQ ID NO: | ACACUCGGUAAGGUUUAAAC |
| | | 381) | (SEQ ID NO: 430) |
| 66 | CasM.291507 | GUUGCAACUUACGCA | TTCAGGGCGACTCGGCGTCCTAA |
| | (SEQ ID NO: | UAGGUGUAAAAUACG | AATCGAGAAAGTGTACATAAGT |
| | 41) | AGUAUUAAAUACUCG | TTTTAACAAAATACGGTAAATAC |
| | | UAUUGCU (SEQ ID NO: | TCTCGGTAAGGTTTTAACGTGCA |
| | | 380) | CATAATAATCCGTGCAACAGGGT |
| | | | TACACTTTTGTGCAATTTT (SEQ |
| | | | ID NO: 431) |
| 67 | CasM.291507 | GUUGCAACUUACGCA | UUCAGGGCGACUCGGCGUCCUA |
| | (SEQ ID NO: | UAGGUGUAAAAUACG | AAAUCGAGAAAGUGUACAUAAG |
| | 41) | AGUAUUAAAUACUCG | UUUUUAACAAAAUACGGUAAAU |
| | | UAUUGCU (SEQ ID NO: | ACUCUCGGUAAGGUUUUAACG |
| | | 380) | (SEQ ID NO: 432) |
| 68 | CasM.293410 | UCAGCUCACAACCUA | TATTAAGGGCGGCTCAGCGTCCT |
| | (SEQ ID NO: | CAUAUGCAUACAAGA | TAAGTCGAGAAAGTATACATAA |
| | 42) | UAUAUCGUUAUUAAA | ATTTCTTATATAGAATAGTAGAT |
| | | UACUCGUAUUGCU | ACTCTCGGCAAGGTATAAACCCT |
| | | (SEQ ID NO: 382) | ACAAATTTAATCCTTGTAGGCAA |
| | | | CTTATATTTGTATTTATTT (SEQ |
| | | | ID NO: 433) |
| 69 | CasM.293410 | UCAGCUCACAACCUA | UAUUAAGGGCGGCUCAGCGUCC |
| | (SEQ ID NO: | CAUAUGCAUACAAGA | UUAAGUCGAGAAAGUAUACAUA |
| | 42) | UAUAUCGUUAUUAAA | AAUUUCUUAUAUAGAAUAGUA |
| | | UACUCGUAUUGCU | GAUACUCUCGGCAAGGUAUAAA |
| | | (SEQ ID NO: 382) | CC (SEQ ID NO: 434) |
| 70 | CasM.295105 | GAUCAUAUCUGCUUG | TTTCGGGCGGCTCGGCGTCCGTA |
| | (SEQ ID NO: | UAUGGGUAUGCUGCG | AACCGAGAAAGTATATGTAAGT |
| | 43) | AGUAUUAAAUACUCG | CTGAATTTATTCAGCGTTAGATA |
| | | UAUUGCU (SEQ ID NO: | CACTCGGTAAGGTTCAAACAATA |
| | | 379) | CATATTCAATCCATGTATTCAGT |
| | | | ATATTTGTACATTTTT (SEQ ID |
| | | | NO: 435) |
| 71 | CasM.295105 | GAUCAUAUCUGCUUG | UUUCGGGCGGCUCGGCGUCCGU |
| | (SEQ ID NO: | UAUGGGUAUGCUGCG | AAACCGAGAAAGUAUAUGUAAG |
| | 43) | AGUAUUAAAUACUCG | UCUGAAUUUAUUCAGCGUUAGA |
| | | UAUUGCU (SEQ ID NO: | UACACUCGGUAAGGUUCAAAC |
| | | 379) | (SEQ ID NO: 436) |
| 72 | CasM.295187 | GAUAUAUCUUGUAUG | ATATTAAGGGCGGCTCAGCGTCC |
| | (SEQ ID NO: | CAUAUGUAGGUUGUG | TTAAGTCGAGAAAGTATACATA |
| | 44) | AGUAUUAAAUACUCG | AATTTCTTATATAGAATAGTAGA |
| | | UAUUGCU (SEQ ID NO: | TACTCTCGGCAAGGTATAAACCC |
| | | 383) | TACAAATTTAATCCTTGTAGGCA |
| | | | ACTTATATTTGTATTTATTT (SEQ |
| | | | ID NO: 437) |
| 73 | CasM.295187 | GAUAUAUCUUGUAUG | AUAUUAAGGGCGGCUCAGCGUC |
| | (SEQ ID NO: | CAUAUGUAGGUUGUG | CUUAAGUCGAGAAAGUAUACAU |
| | 44) | AGUAUUAAAUACUCG | AAAUUUCUUAUAUAGAAUAGU |
| | | UAUUGCU (SEQ ID NO: | AGAUACUCUCGGCAAGGUAUAA |
| | | 383) | AC (SEQ ID NO: 438) |
| 74 | CasM.295929 | GUUGCAAUGAACGUA | AAACAAGGGCGGCTCAACGTCC |
| | (SEQ ID NO: | UGUGCAUGAGGUGUG | TAGAATCGAGAAAGTATGCGTA |
| | 45) | AGUAUUAAAUACUCG | AGACTTATTTATTGAGCGGTAGA |
| | | UAUUGCU (SEQ ID NO: | TACTCTCGGTAAGGTATAAATTC |
| | | 384) | CACAATGAAAATCCTGTGGACA |
| | | | CCGTATAATATGTGCATGTTT |
| | | | (SEQ ID NO: 439) |
| 75 | CasM.295929 | GUUGCAAUGAACGUA | AAACAAGGGCGGCUCAACGUCC |
| | (SEQ ID NO: | UGUGCAUGAGGUGUG | UAGAAUCGAGAAAGUAUGCGUA |
| | 45) | AGUAUUAAAUACUCG | AGACUUAUUUAUUGAGCGGUAG |
| | | UAUUGCU (SEQ ID NO: | AUACUCUCGGUAAGGUAUAAAU |
| | | 384) | UC (SEQ ID NO: 440) |

TABLE 26
Exemplary sgRNAs for CasM Effector Proteins

| Comp. | Effector | |
|---|---|---|
| No | protein | SgRNA |
| 31 | CasM.19924 | UGGGGCAGUUGGUUGCCCUUAGCCUGAGGCAUU |
| | (SEQ ID NO: | UAUUGCACUCGGGAAGUACCAUUUCUCAGAAAU |
| | 22) | GGUACAUCCAACUAUUAAAUACUCGUAUUGCU |
| | | ((SEQ ID NO: 441) |
| 33 | CasM.19952 | UGGGGCAGUUGGUUGCCCUUAGCCUGAGGCAUU |
| | (SEQ ID NO: | UAUUGCACUCGGGAAGUACCAUUUCUCAGAAAU |
| | 23) | GGUACAUCCAACUAUUAAAUACUCGUAUUGCU |
| | | (SEQ ID NO: 441) |
| 35 | CasM.274559 | AUGGGGCAGUUGGUUGCCCUUAGCCUGAGGCAU |
| | (SEQ ID NO: | UUAAUGCACUCGGGAAGUACCUUUUCUCAGAAA |
| | 24) | UGGUACAUCCAACUAUUAAAUACUCGUAUUGCU |
| | | (SEQ ID NO: 442) |
| 37 | CasM.286251 | AUGGGGCAGUUGGUUGCCCUUAGCCUGAGGAAU |
| | (SEQ ID NO: | UUAAUUCACUCGGGAAGUACCUUUCUCAUGAAA |
| | 25) | UGGUACAUCCAACUAUUAAAUACUCGUAUUGCU |
| | | (SEQ ID NO: 443) |
| 39 | CasM.288480 | UGGGGCAGUUGGUUGCCCUUAGCCUGAGGCAUU |
| | (SEQ ID NO: | UAUUGCACUCGGGAAGUACCAUUUCUCAGAAAU |
| | 26) | GGUACAUCCAACUAUUAAAUACUCGUAUUGCU |
| | | (SEQ ID NO: 441) |
| 42 | CasM.289206 | AUGGGGCAGUUGGUUGCCCUUAGCCUGAGGCAU |
| | (SEQ ID NO: | UUAAUGCACUCGGGAAGUACCUUUUCUCAGAAA |
| | 28) | UGGUACAUCCAACUAUUAAAUACUCGUAUUGCU |
| | | (SEQ ID NO: 442) |
| 44 | CasM.290598 | AUGGGGCAGUUGGUUGCCCUUAGCCUGAGGCAU |
| | (SEQ ID NO: | UUAAUGCACUCGGGAAGUACCUUUUCUCAGAAA |
| | 29) | UGGUACAUCCAACUAUUAAAUACUCGUAUUGCU |
| | | (SEQ ID NO: 442) |
| 46 | CasM.290816 | AUGGGGCAGUUGGAUGCCCUUAGCCUGAGGCAU |
| | (SEQ ID NO: | UUAUUGCACUCGGGAAGUACCUUUUCUCAGAAA |
| | 30 | UGGUACAUCCAACUAUUAAAUACUCGUAUUGCU |
| | | (SEQ ID NO: 444) |
| 48 | CasM.295071 | AUGGGGCAGUUGGUUGCCCUUAGCCUGAGGAAU |
| | (SEQ ID NO: | UUAAUUCACUCGGGAAGUACCUUUCUCAUGAAA |
| | 31) | UGGUACAUCCAACUAUUAAAUACUCGUAUUGCU |
| | | (SEQ ID NO: 443) |
| 51 | CasM.295231 | AUGGGGCAGUUGGAUGCCCUUAGCCUGAGGCAU |
| | (SEQ ID NO: | UUAUUGCACUCGGGAAGUACCUUUUCUCAGAAA |
| | 32 | UGGUACAUCCAACUAUUAAAUACUCGUAUUGCU |
| | | (SEQ ID NO: 444) |
| 53 | CasM.292139 | TTATTAGAAATGAAATATTTTCTAATGGGGTTG |
| | (SEQ ID NO: | TTGGAAAGAGCTTTTACTGAAATTTGTAAAGGT |
| | 33) | GCCCTGAACTTGAGAATTGAAAAATTACTCGAG |
| | | GAAATGGTACATCCAACTATTAAATACTCGTAT |
| | | TGCT (SEQ ID NO: 445) |

TABLE 27
Observed Cis Cleavage for Effector Protein/Guide Combinations

| | | cis- | | | |
|---|---|---|---|---|---|
| Comp. | | cleavage | | | |
| No: | Effector Protein | (y/n) | crRNA # | tracrRNA # | sgRNA # |
| 1 | CasM.298706 | Y | R4879 (SEQ ID | R4935 (SEQ ID | — |
| | (SEQ ID NO: 1) | | NO: 347) | NO: 385) | |
| 4 | CasM.284933 | Y | R4841 (SEQ ID | R4902 (SEQ ID | — |
| | (SEQ ID NO: 4) | | NO: 350) | NO: 388) | |
| 13 | CasM.297894 | Y | R4987 (SEQ ID | R4904 (SEQ ID | — |
| | (SEQ ID NO: 13) | | NO: 358) | NO: 396) | |
| 14 | CasM.291449 | N | R4875 (SEQ ID | R4939 (SEQ ID | — |
| | (SEQ ID NO: 14) | | NO: 359) | NO: 397) | |
| 15 | CasM.291449 | N | R4875 (SEQ ID | R4938 (SEQ ID | — |
| | (SEQ ID NO: 14) | | NO: 359) | NO: 398) | |
| 16 | CasM.297599 | Y | R4876(SEQ ID | R4892 (SEQ ID | — |
| | (SEQ ID NO: 15) | | NO: 360) | NO: 399) | |
| 17 | CasM.297599 | Y | R4876 (SEQ ID | R4942 (SEQ ID | — |
| | (SEQ ID NO: 15) | | NO: 360) | NO: 400) | |
| 23 | CasM.292335 | Y | R4851 (SEQ ID | R4907 (SEQ ID | — |
| | (SEQ ID NO: 18) | | NO: 363) | NO: 406) | |
| 24 | CasM.293576 | Y | R4852 (SEQ ID | R4896(SEQ ID | — |
| | (SEQ ID NO: 19) | | NO: 364) | NO: 407) | |
| 28 | CasM.298538 | Y | R4854 (SEQ ID | R4897 (SEQ ID | — |
| | (SEQ ID NO: 21) | | NO: 365) | NO: 411) | |
| 30 | CasM.19924 | Y | R4855 (SEQ ID | R4893 (SEQ ID | — |
| | (SEQ ID NO: 22) | | NO: 366) | NO: 413) | |
| 31 | CasM.19924 | Y | — | — | R4886 |
| | (SEQ ID NO: 22) | | | | ((SEQ ID |
| | | | | | NO: 441) |
| 32 | CasM.19952 | Y | R4856 (SEQ ID | R4893 (SEQ ID | — |
| | (SEQ ID NO: 23) | | NO: 367) | NO: 413) | |
| 33 | CasM.19952 | Y | — | — | R4886 |
| | (SEQ ID NO: 23) | | | | (SEQ ID |
| | | | | | NO: 441) |
| 34 | CasM.274559 | Y | R4857 (SEQ ID | R4894 (SEQ ID | — |
| | (SEQ ID NO: 24) | | NO: 368) | NO: 414) | |
| 35 | CasM.274559 | Y | — | — | R4887(SEQ |
| | (SEQ ID NO: 24) | | | | ID NO: 442) |
| 36 | CasM.286251 | Y | R4858 (SEQ ID | R4910 (SEQ ID | — |
| | (SEQ ID NO: 25) | | NO: 369) | NO: 415) | |
| 37 | CasM.286251 | Y | — | — | R4882 |
| | (SEQ ID NO: 25) | | | | (SEQ ID |
| | | | | | NO: 443) |
| 39 | CasM.288480 | Y | — | — | R4886 |
| | (SEQ ID NO: 26) | | | | (SEQ ID |
| | | | | | NO: 441) |
| 41 | CasM.289206 | Y | R4861 (SEQ ID | R4894 (SEQ ID | — |
| | 289206 (SEQ ID | | NO: 372) | NO: 414) | |
| | NO: 28) | | | | |
| 42 | CasM.289206 | Y | — | — | R4887 |
| | (SEQ ID NO: 28) | | | | (SEQ ID |
| | | | | | NO: 442) |
| 43 | CasM.290598 | Y | R4862 (SEQ ID | R4894 (SEQ ID | — |
| | (SEQ ID NO: 29) | | NO: 373) | NO: 414) | |
| 45 | CasM.290816 | Y | R4863 (SEQ ID | R4912 (SEQ ID | — |
| | (SEQ ID NO: 30) | | NO: 374) | NO: 417) | |
| 48 | CasM.295071 | Y | — | — | R4882(SEQ |
| | (SEQ ID NO: 31) | | | | ID NO: 443) |
| 50 | CasM.295231(SE | Y | — | — | R4884 |
| | Q ID NO: 32) | | | | (SEQ ID |
| | | | | | NO: 444) |
| 54 | CasM.279423 | Y | R4857 (SEQ ID | R4894 (SEQ ID | |
| | (SEQ ID NO: 34) | | NO: 368) | NO: 414) | |
| 71 | CasM.295105 | Y | R4872(SEQ ID | R4925 (SEQ ID | |
| | (SEQ ID NO: 43) | | NO: 379) | NO: 436) | |
| 72 | CasM.295187 | Y | R4873 (SEQ ID | R4945(SEQ ID | |
| | (SEQ ID NO: 44) | | NO: 383) | NO: 437) | |
| 74 | CasM.295929 | Y | R4874 (SEQ ID | R4928 (SEQ ID | |
| | (SEQ ID NO: 45) | | NO: 384) | NO: 439) | |
| 75 | CasM.295929 | Y | R4874 (SEQ ID | R4927 (SEQ ID | |
| | (SEQ ID NO: 45) | | NO: 384) | NO: 440) | |

TABLE 28
Exemplary PAM Sequences

| | Effector | | |
|---|---|---|---|
| Composition | Protein | Amino Acid | PAM |
| No | Name | SEQ ID NO: | Sequence |
| 1 | CasM.298706 | 1 | CTT |
| 13 | CasM.297894 | 13 | CTT |
| 16 | CasM.297599 | 15 | CC |
| 17 | CasM.297599 | 15 | CC |
| 23 | CasM.292335 | 18 | CC |
| 24 | CasM.293576 | 19 | CC |
| 28 | CasM.298538 | 21 | TC |
| 30 | CasM.19924 | 22 | TCG |
| 31 | CasM.19924 | 22 | GCG |
| 32 | CasM.19952 | 23 | TCG, TTG, |
| | | | GCG, GTG |
| 33 | CasM.19952 | 23 | TCG, TTG, |
| | | | GCG, GTG |
| 34 | CasM.274559 | 24 | TCG |
| 35 | CasM.274559 | 24 | TCG |
| 36 | CasM.286251 | 25 | ATTA, ATTG, |
| | | | GTTA, GTTG |
| 37 | CasM.286251 | 25 | ATTA, ATTG, |
| | | | GTTA, GTTG |
| 39 | CasM.288480 | 26 | TCG |
| 41 | CasM.289206 | 28 | ATTA, ATTG, |
| | | | GTTA, GTTG |
| 42 | CasM.289206 | 28 | ATTA, ATTG, |
| | | | GTTA, GTTG |
| 43 | CasM.290598 | 29 | ATTG, ACTG, |
| | | | GTTG, GCTG |
| 46 | CasM.290816 | 30 | TCG |
| 48 | CasM.295071 | 31 | ATTA, ATTG, |
| | | | GTTA, GTTG |
| 50 | CasM.295231 | 32 | TCG or GCG |
| 54 | CasM.279423 | 34 | ATTA, ATTG, |
| | | | GTTA, GTTG |
| 71 | CasM.295105 | 43 | TTC |
| 72 | CasM.295187 | 44 | TTC |
| 74 | CasM.295929 | 45 | TTT, TTC |
| 75 | CasM.295929 | 45 | TTT, TTC |

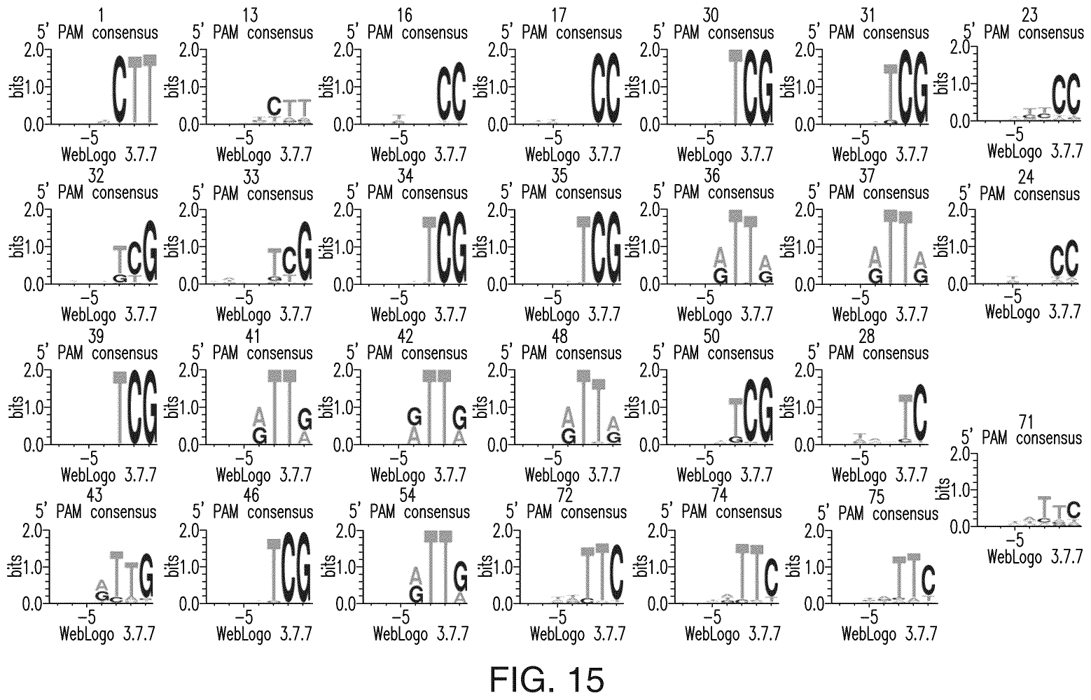

FIG. 15 illustrates the composition of the sequences derived from libraries digested with RNP complexes comprising the denoted effector proteins. As shown in FIG. 15, examination of the PFM derived WebLogos (FIG. 15) revealed the presence of enriched 5′ PAM consensus sequences for the various effector proteins.

### Example 14. Generation of CAR T Cells Directed to CD-19 and Cytotoxicity to CD-19-Expressing Cells

This example demonstrates the generation of CART cells by integration of a CD-19 specific CAR into the TRAC locus of T cells using RNP complexes of CasΦ and a TRAC specific guide RNA, and the cytotoxic activity of such cells on CD19-expressing NALM-6 cells.

### Thawing, Resting and Activating T Cells

In a 15 ml falcon tube, 100 μg/ml DNase I (100 μl) was added to 9 ml T Cell Media and pre-warm in a 37° C. cell culture incubator for 15-20 mins. A vial of frozen Pan T cells (STEMCELL Technologies; Cat #70024) containing 2×10⁷ cells per vial were thawed in a 37° C. water bath. Cells were slowly added using a 1000 ul micropipette to the pre-warmed media containing DNase I, and incubated at 37° C. and 5% CO2 for 1 hour. After an hour, the tubes were centrifuged at 1350 rpm for 5 mins. The media was removed and 5 ml of fresh pre-warmed T Cell Media was added. Cells were counted (1.5×10⁷ cells counted). With a loosen cap, the tubes were placed on a rack and allowed to rest overnight at 37° C. and 5% CO₂. Based on the cell count, cells were resuspended at a concentration of 1×10⁶ cells/ml and transferred to a fresh, sterile T-75 flask. Dynabeads (3 beads per cell) were added and incubated at 37° C. and 5% CO₂ for 3 days.

### Transfection of RNP Complexes

RNP complexes were generated by mixing 500 pmol TRAC CasΦ guide RNA (CUUUCAAGACUAAUAGAUUGCUCCUUACGAGGAGACGAGUCUCUCAGCUGGUACA C (SEQ ID NO: 1382)) with 250 pmol CasΦ.12 for an RNA:Effector Protein ratio of 2:1, an incubated at RT for 30 mins. Activated T cells were transferred from T-75 flask to a 15 ml tube and all Dynabeads were removed from the cells (debeading) by placing the tube in a magnetic stand for 5 mins. Cells were resuspended in P3 solution at a concentration of 2.5×10⁷ cells/ml and 20 μl of this suspension was used for each reaction. The RNPs were mixed with the cells just before the electroporation. 20 μl of this mixture was added to each well of the nucleofection plate and electroporated. After nucleofection, 180 ul of pre-warmed T cell media was added to all the reaction wells and allowed to sit at 37° C. and 5% CO₂ for 10 mins. After this recovery incubation, the electroporated cells were transferred to a 48-well plate, including combining 2 wells of the same condition from the plate into one well of the destination 48-well plate so that the final volume in each well is 500 μl. Cells were incubated at 37° C. and 5% CO₂ for 2 hours before AAV transduction.

### AAV Transduction

Following transfection of RNP complexes, AAV6 particles containing a donor nucleotide sequence encoding either a CD19-CAR or a GFP marker were added at an 1×10⁵ MOI of the electroporated T cells. The plates were placed back into 37° C. and 5% CO₂ and analyzed after 5 days of culturing.

### Analysis of CD19-CAR Integrationbyflow Cytometry

Cells were resuspend in the media and 150 μl was transferred to a fresh plate. The remaining approximately 50 μl cells were used for genomic DNA extraction. The new plate was centrifuged at 1500 rpm for 5 mins and the media was discarded.

In order to assess the number of live/dead cells, Zombie NIR Fixable Viability Dye was diluted 1:1000 and then 100 μl per sample was added, resuspended and incubated at RT for 15 min in the dark. 150 μl of PBS was added to the wells and pipette mixed to wash. The plate was spun at 1500 rpm for 5 min.

In order to stain the cells, extracellular staining was conducted as follows. Blocking—0.5 μl/sample normal goat IgG was added to block non-specific cell surface receptors in FACS buffer. Samples were incubated for 20 mins at 4° C. and washed. CD19-CAR 1° Ab staining—1 μl/sample of Biotin-tagged mouse IgG was added in FACS Buffer to stain the CD19-CAR construct. Samples were incubated for 25 mins at 4° C. and washed. CD19-CAR 2° Ab and CD3 staining—0.33 μl Streptavidin-PE and 5 μl anti-CD3 antibody (APC) was added in FACS Buffer to each sample. Samples were incubated for 25 mins at 4° C. and washed. All samples were spun at 1500 rpm for 5 mins and cells were resuspended in 100 μl FACS Buffer and run on flow cytometer.

Voltages of lasers on the flow cytometer were set in accordance with compensation controls. All stained samples were run using these voltages. Gates were set using isotype controls for the antibodies and FMO control for the L/D Zombie NIR stain. Flow data was analyzed using FlowJo v10 and graphs were plotted using GraphPad Prism.

### Enrichment of Cd3⁻ Cells —Magnetic Bead Separation

CD3⁻ cells were separated using the MojoSort™ Human CD3 Selection Kit according to manufacturer's instructions.

### Cell Killing Assay —LDH Release

The LDH Assay was performed according to manufacturer's instructions. Briefly, Target Cells (NALM6), Effector Cells (CD19-CAR T cells) and controls were added to a U-bottom 96-well plate in 100 μl media and incubated at 37° C. for 24 hours. To make CytoTox 96 Reagent: Assay Buffer from kit was thawed, and 12 ml was added to one amber bottle of Substrate Mix. Assay buffer was made fresh before every readout. After 24 hours, the assay plate was spun at 1500 rpm for 5 mins. 50 μl from each well was removed and transferred to a new flat bottom 96-well plate. 50 μl of the CytoTox 96 Reagent was added and incubated in the dark at RT for 30 mins. 50 μl Stop Solution was added and read at 490 nm on a spectrophotometer within 1 hour. Specific cytotoxicity of the NALM6 cells was calculated by the following formula: % Cytotoxicity=[(Experimental−Effector Spont. Release-Target Spont. Release)/(Target Max. Release−Target Spont. Release)]*100

### CD3⁻ Cell Enrichment

The percentage of CD3⁻ cells increased from 87.7% before sorting to 97.2% after sorting.

### Efficiencies of Integration

In the CD19-CAR samples, approximately 30% CAR integration was observed in CD3⁻ or TRAC KO subset of the T cells. In the GFP samples, approximately 49% or 60% of GFP integration was observed in the CD3⁻ or TRAC KO subset of the T cells.

### Cytotoxicity

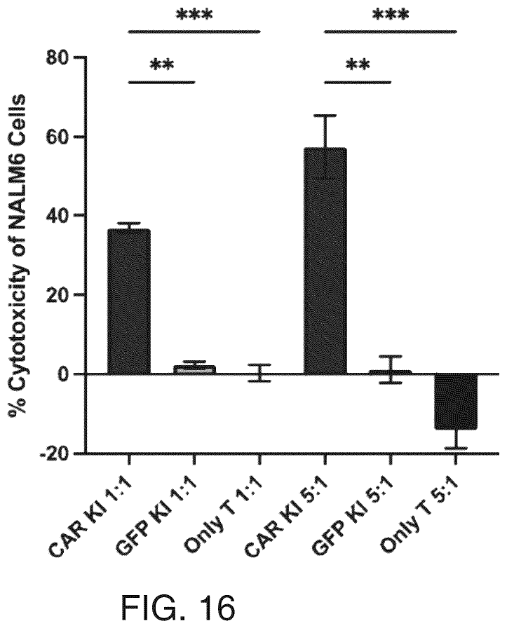

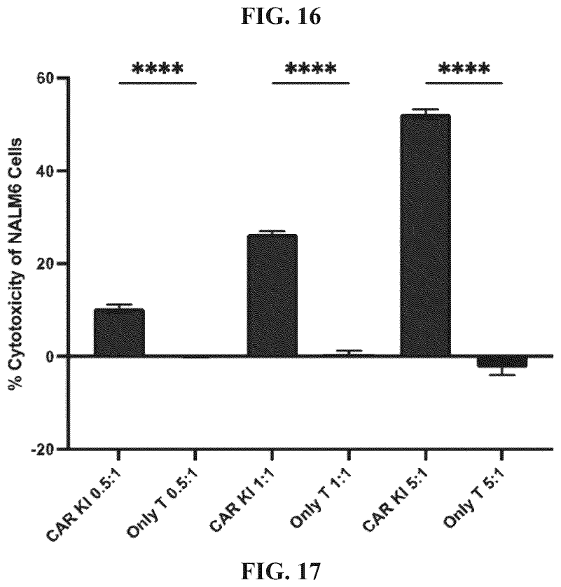

Exemplary results are shown in FIG. 16 and FIG. 17. In all experiments, the CD19-CAR T cells showed significantly higher cell killing than the GFP⁺ or the control T cells in a dose dependent manor. For example, in a first experiment, at a ratio of 1:1 (Effector Cells:Target Cells), there was approximately 40% cytotoxicity and at a ratio of 5:1, cytotoxicity went up to approximately 60%. In a second experiment, at ratios of 0.5:1, 1:1, and 5:1 there was approx. 10%, 30%, and 50% cytotoxicity, respectively.

### Example 15. Production of CAR T-Cells with AAV Vector Encoding an Effector Protein, Guide RNAs Targeting TRAC, B2M and CIITA, and Donor Sequence Encoding a CAR

An AAV vector is constructed to contain multiple nucleotide sequences between its ITRs, wherein these nucleotide sequences provide or encode, in a 5′ to 3′ direction, a donor nucleic acid encoding a CAR and nucleotide sequences flacking the CAR encoding sequence directing integration of the donor into the TRAC gene, a first promoter, a guide nucleic acid having a sequence complementary to an equal length portion of a TRAC encoding sequence, a second promoter, a guide nucleic acid having a sequence complementary to an equal length portion of a B2M encoding sequence, a third promoter, a guide nucleic acid having a sequence complementary to an equal length portion of a CIITA encoding sequence, a fourth promoter, an effector protein having a nuclear localization signal, and a poly A tail. The size of the donor nucleic acid is about 1 kb. The size of the Cas effector is less than 600 amino acids. The total length of the AAV vector, including the ITRs, is about 4.8 kb. The AAV vector is expressed with supporting plasmids to produce AAV particles containing the AAV vector. T cells from a healthy donor subject are contacted with the AAV particles. After about 48 hours, DNA or RNA is isolated from the transduced cells. Expression of the CAR and reduced expression of the TRAC, B2M and CIITA genes is confirmed by Q-PCR.

### Example 16. Indel Generation in Eukaryotic Cells with CasM.265466 and Guides Targeting B2M

Guides targeting exon 1 or exon 2 of B2M were tested with Cas 265466 (SEQ ID NO: 2435) for the ability to produce indels in primary T Cells. 16 modified guide RNA sequences (i.e., a phosphorothioate bond between the nucleotides, a 2′-OMe modification) directed to various target sequences were tested and their ability to introduce indels was measured. Briefly, about 30×10⁶ T cells were electroporated with a mixture of mRNA of the Cas 265466 (5 μg) and different guides (500 μmol). The transfected cells were incubated for ˜72 hours to allow for indel formation followed by DNA extraction.

After the 72-hour incubation, a portion of the cells were incubated with a Live/Dead cell stain and a B2M antibody for fluorescence-activated cell sorting (FACS) analysis. Indels were detected by next generation sequencing (NGS) of PCR amplicons at the targeted loci 3 days and 7 days post-transfection. Indel percentage was calculated as the fraction of sequencing reads containing insertions or deletions relative to an unedited reference sequence. The effector proteins Cas9 and Cas 12a were used as a positive control. The results are summarized in TABLE 29. An analysis of the results indicates that the effector protein CasM.265466 mRNA and the guides targeting B32M gene can be used for editing the gene.

TABLE 29
Exemplary modified guides for B2M editing in T cells

| | | | | | | Sequence | |
|---|---|---|---|---|---|---|---|
| | | | | | FACS | Analysis | |
| Effector | | gRNA | | | %-ve | % | % |
| Protein | 5′ | SEQ | | | Cells | Indels | Indels |
| SEQ ID | PAM | ID | RNA | Target | (3 | (3 | (7 |
| NO | Seq | NO: | Modification | Gene | days) | days) | days) |
| 1 | TGTG | 2436 | mA*mC*mA*GCUUAU | B2M | • | •• | •• |
| | | | UUGGAAGCUGAAAUG | exon: | | | |
| | | | UGAGGUUUAUAACAC | #1 | | | |
| | | | UCACAAGAAUCCUGA | | | | |
| | | | AAAAGGAUGCCAAAC | | | | |
| | | | CUCGCGCUACUCUCU | | | | |
| | | | CUmU*mU*mC | | | | |
| 1 | TCTG | 2437 | mA*mC*mA*GCUUAU | B2M | •• | ••• | •• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | GGUUUCAUCCAUCCG | | | | |
| | | | ACmA*mU*mU | | | | |
| 1 | TGTA | 2438 | mA*mC*mA*GCUUAU | B2M | ••• | ••• | ••• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | CUACACUGAAUUCAC | | | | |
| | | | CCmC*mC*mA | | | | |
| 1 | TCTA | 2439 | mA*mC*mA*GCUUAU | B2M | • | •• | •• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | UCUCUUGUACUACAC | | | | |
| | | | UGmA*mA*mU | | | | |
| 1 | TTTA | 2440 | mA*mC*mA*GCUUAU | B2M | • | •• | •• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | CUCACGUCAUCCAGC | | | | |
| | | | AGmA*mG*mA | | | | |
| 1 | TATG | 2441 | mA*mC*mA*GCUUAU | B2M | •• | ••• | •• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | UGUCUGGGUUUCAUC | | | | |
| | | | CAmU*mC*mC | | | | |
| 1 | TATG | 2442 | mA*mC*mA*GCUUAU | B2M | • | • | • |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | CCUGCCGUGUGAACC | | | | |
| | | | AUmG*mU*mG | | | | |
| 1 | TTTG | 2443 | mA*mC*mA*GCUUAU | B2M | • | • | • |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | UCACAGCCCAAGAUA | | | | |
| | | | GUmU*mA*mA | | | | |
| 1 | TGTG | 2444 | mA*mC*mA*GCUUAU | B2M | •• | •• | •• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | ACUUUGUCACAGCCC | | | | |
| | | | AAmG*mA*mU | | | | |
| 1 | TGTG | 2445 | mA*mC*mA*GCUUAU | B2M | • | • | • |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | UCUGGGUUUCAUCCA | | | | |
| | | | UCmC*mG*mA | | | | |
| 1 | TGTG | 2446 | mA*mC*mA*GCUUAU | B2M | • | • | • |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | AACCAUGUGACUUUG | | | | |
| | | | UCmA*mC*mA | | | | |
| 1 | TCTG | 2447 | mA*mC*mA*GCUUAU | B2M | •• | ••• | ••• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | AAUGCUCCACUUUUU | | | | |
| | | | CAmA*mU*mU | | | | |
| 1 | TTTG | 2448 | mA*mC*mA*GCUUAU | B2M | • | •• | •• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | ACUUUCCAUUCUCUG | | | | |
| | | | CUmG*mG*mA | | | | |
| 1 | TGTG | 2449 | mA*mC*mA*GCUUAU | B2M | ••• | ••• | ••• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | ACAAAGUCACAUGGU | | | | |
| | | | UCmA*mC*mA | | | | |
| 1 | TGTA | 2450 | mA*mC*mA*GCUUAU | B2M | • | • | • |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | GUACAAGAGAUAGAA | | | | |
| | | | AGmA*mC*mC | | | | |
| 1 | TCTG | 2451 | mA*mC*mA*GCUUAU | B2M | ••• | ••• | ••• |
| | | | UUGGAAGCUGAAAUG | exon: | | | |
| | | | UGAGGUUUAUAACAC | #2 | | | |
| | | | UCACAAGAAUCCUGA | | | | |
| | | | AAAAGGAUGCCAAAC | | | | |
| | | | CUGGAUGACGUGAGU | | | | |
| | | | AAmA*mC*mC | | | | |

RNA Modification: “*” represents a phosphorothioate bond between the nucleotides, “m” denotes a 2′-OMe modification.
Magnitude of data: “•••” represents a value >40, “••” represents a value between ≤40 and ≥20, “•” represents a value <20.

### Example 17. Indel Generation in Eukaryotic Cells with CasM.265466 and Guides Targeting TRAC

Guides targeting exon 1, exon 2 and exon 3 of TRAC were tested with Cas 265466 (SEQ ID NO: 2435) for the ability to produce indels in primary T Cells. 33 modified guide RNA sequences (i.e., a phosphorothioate bond between the nucleotides, a 2′-OMe modification) directed to various target sequences were tested and their ability to introduce indels was measured. Briefly, about 30×10⁶ T cells were electroporated with a mixture of mRNA of the Cas 265466 (5 μg) and different guides (500 μmol). The transfected cells were incubated for ˜72 hours to allow for indel formation followed by DNA extraction.

After the 72-hour incubation, a portion of the cells were incubated with a Live/Dead cell stain and a CD3 antibody for fluorescence-activated cell sorting (FACS) analysis. Indels were detected by next generation sequencing (NGS) of PCR amplicons at the targeted loci 3 days post-transfection. Indel percentage was calculated as the fraction of sequencing reads containing insertions or deletions relative to an unedited reference sequence. The effector proteins Cas9 and Cas 12a were used as a positive control. The results are summarized in TABLE 30. An analysis of the results indicates that the effector protein CasM.265466 mRNA and the guides targeting TRAC gene can be used for editing the gene.

TABLE 30
Exemplary modified guides for TRAC editing in T cells

| | | | | | FACS | |
|---|---|---|---|---|---|---|
| Effector | | | | | Analysis | Seq. |
| Protein | 5′ | gRNA | | | %-ve | Analysis |
| SEQ ID | PAM | SEQ ID | | Target | Cells | % Indels |
| NO | Seq | NO: | RNA Modification | Gene | (3 days) | (3 days) |
| 1 | TGTG | 2452 | mA*mC*mA*GCUUAUU | TRAC | ••• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUCACA | | | |
| | | | AAGUAAGGAUUCmU*m | | | |
| | | | G*mA | | | |
| 1 | TCTA | 2453 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUGGAC | | | |
| | | | UUCAAGAGCAACmA*m | | | |
| | | | G*mU | | | |
| 1 | TTTG | 2454 | mA*mC*mA*GCUUAUU | TRAC | • | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACAUUCU | | | |
| | | | CAAACAAAUGUGmU*m | | | |
| | | | C*mA | | | |
| 1 | TCTG | 2455 | mA*mC*mA*GCUUAUU | TRAC | •• | •• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACACUUU | | | |
| | | | GCAUGUGCAAACmG*m | | | |
| | | | C*mC | | | |
| 1 | TGTG | 2456 | mA*mC*mA*GCUUAUU | TRAC | • | •• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACCAAAC | | | |
| | | | GCCUUCAACAACmA*m | | | |
| | | | G*mC | | | |
| 1 | TGTG | 2457 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUAUAU | | | |
| | | | CACAGACAAAACmU*m | | | |
| | | | G*mU | | | |
| 1 | TCTA | 2458 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACAAUCC | | | |
| | | | AGUGACAAGUCUmG*m | | | |
| | | | U*mC | | | |
| 1 | TCTG | 2459 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACAUGUG | | | |
| | | | UAUAUCACAGACmA*m | | | |
| | | | A*mA | | | |
| 1 | TTTG | 2460 | mA*mC*mA*GCUUAUU | TRAC | •• | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACCAUGU | | | |
| | | | GCAAACGCCUUCmA*m | | | |
| | | | A*mC | | | |
| 1 | TATA | 2461 | mA*mC*mA*GCUUAUU | TRAC | ••• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUCACA | | | |
| | | | GACAAAACUGUGmC*m | | | |
| | | | U*mA | | | |
| 1 | TGTA | 2462 | mA*mC*mA*GCUUAUU | TRAC | ••• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUAUCA | | | |
| | | | CAGACAAAACUGmU*m | | | |
| | | | G*mC | | | |
| 1 | TCTG | 2463 | mA*mC*mA*GCUUAUU | TRAC | •• | •• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUCUGC | | | |
| | | | CUAUUCACCGAUmU*m | | | |
| | | | U*mU | | | |
| 1 | TGTG | 2464 | mA*mC*mA*GCUUAUU | TRAC | •• | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACGCCUG | | | |
| | | | GAGCAACAAAUCmU*m | | | |
| | | | G*mA | | | |
| 1 | TGTA | 2465 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACCCAGC | | | |
| | | | UGAGAGACUCUAmA*m | | | |
| | | | A*mU | | | |
| 1 | TCTG | 2466 | mA*mC*mA*GCUUAUU | TRAC | • | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACCCUAU | | | |
| | | | UCACCGAUUUUGmA*m | | | |
| | | | U*mU | | | |
| 1 | TGTG | 2467 | mA*mC*mA*GCUUAUU | TRAC | •• | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACCUAGA | | | |
| | | | CAUGAGGUCUAUmG*m | | | |
| | | | G*mA | | | |
| 1 | TATG | 2468 | mA*mC*mA*GCUUAUU | TRAC | •• | •• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACGACUU | | | |
| | | | CAAGAGCAACAGmU*m | | | |
| | | | G*mC | | | |
| 1 | TCTA | 2469 | mA*mC*mA*GCUUAUU | TRAC | • | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACGCACA | | | |
| | | | GUUUUGUCUGUGmA*m | | | |
| | | | U*mA | | | |
| 1 | TTTG | 2470 | mA*mC*mA*GCUUAUU | TRAC | • | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACAGAAU | | | |
| | | | CAAAAUCGGUGAmA*m | | | |
| | | | U*mA | | | |
| 1 | TATA | 2471 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACCACAU | | | |
| | | | CAGAAUCCUUACmU*m | | | |
| | | | U*mU | | | |
| 1 | TCTG | 2472 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUGAUA | | | |
| | | | UACACAUCAGAAmU*m | | | |
| | | | C*mC | | | |
| 1 | TGTG | 2473 | mA*mC*mA*GCUUAUU | TRAC | • | •• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACACACA | | | |
| | | | UUUGUUUGAGAAmU*m | | | |
| | | | C*mA | | | |
| 1 | TTTG | 2474 | mA*mC*mA*GCUUAUU | TRAC | • | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUGACA | | | |
| | | | CAUUUGUUUGAGmA*m | | | |
| | | | A*mU | | | |
| 1 | TTTA | 2475 | mA*mC*mA*GCUUAUU | TRAC | ••• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACGAGUC | | | |
| | | | UCUCAGCUGGUAmC*m | | | |
| | | | A*mC | | | |
| 1 | TTTG | 2476 | mA*mC*mA*GCUUAUU | TRAC | ••• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUUGCU | | | |
| | | | CCAGGCCACAGCmA*m | | | |
| | | | C*mU | | | |
| 1 | TTTG | 2477 | mA*mC*mA*GCUUAUU | TRAC | • | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACCACAU | | | |
| | | | GCAAAGUCAGAUmU*m | | | |
| | | | U*mG | | | |
| 1 | TTTG | 2478 | mA*mC*mA*GCUUAUU | TRAC | •• | •• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUUUGA | | | |
| | | | GAAUCAAAAUCGmG*m | | | |
| | | | U*mG | | | |
| 1 | TGTG | 2479 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACAUAUA | | | |
| | | | CACAUCAGAAUCmC*m | | | |
| | | | U*mU | | | |
| 1 | TCTG | 2480 | mA*mC*mA*GCUUAUU | TRAC | • | • |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACGAAUA | | | |
| | | | AUGCUGUUGUUGmA*m | | | |
| | | | A*mG | | | |
| 1 | TTTG | 2481 | mA*mC*mA*GCUUAUU | TRAC | •• | ••• |
| | | | UGGAAGCUGAAAUGUG | exon: | | |
| | | | AGGUUUAUAACACUCA | #1 | | |
| | | | CAAGAAUCCUGAAAAA | | | |
| | | | GGAUGCCAAACUCUGU | | | |
| | | | GAUAUACACAUCmA*m | | | |
| | | | G*mA | | | |
| 1 | TGTG | 2482 | mA*mC*mA*GCUUAUU | TRAC | • | •• |
| | | | UGGAAGCUGAAAUGUG | exon: | | |
| | | | AGGUUUAUAACACUCA | #2 | | |
| | | | CAAGAAUCCUGAAAAA | | | |
| | | | GGAUGCCAAACAUGUC | | | |
| | | | AAGCUGGUCGAGmA*m | | | |
| | | | A*mA | | | |
| 1 | TCTG | 2483 | mA*mC*mA*GCUUAUU | TRAC | • | •• |
| | | | UGGAAGCUGAAAUGUG | exon: | | |
| | | | AGGUUUAUAACACUCA | #3 | | |
| | | | CAAGAAUCCUGAAAAA | | | |
| | | | GGAUGCCAAACCUCAU | | | |
| | | | GACGCUGCGGCUmG*m | | | |
| | | | U*mG | | | |
| 1 | TTTA | 2484 | mA*mC*mA*GCUUAUU | TRAC | ••• | ••• |
| | | | UGGAAGCUGAAAUGUG | exon: | | |
| | | | AGGUUUAUAACACUCA | #3 | | |
| | | | CAAGAAUCCUGAAAAA | | | |
| | | | GGAUGCCAAACAUCUG | | | |
| | | | CUCAUGACGCUGmC*m | | | |
| | | | G*mG | | | |

RNA Modification: “*” represents a phosphorothioate bond between the nucleotides, “m” denotes a 2′-OMe modification.
Magnitude of data: “•••” represents a value >45, “••” represents a value between ≤45 and ≥20, “•” represents a value <20.

### Example 18. Indel Generation in Eukaryotic Cells with CasM.265466 and Guides Targeting CITTA

Guides targeting exon 1, exon 2 and exon 3 of CIITA were tested with Cas 265466 (SEQ ID NO: 2435) for the ability to produce indels in primary T Cells. 27 modified guide RNA sequences (i.e., a phosphorothioate bond between the nucleotides, a 2′-OMe modification) directed to various target sequences were tested and their ability to introduce indels was measured. Briefly, 30×10⁶ T cells were electroporated with a mixture of mRNA of the Cas 265466 (5 μg) and different guides (500 pmol The transfected cells were incubated for ˜72 hours to allow for indel formation followed by DNA extraction.

After the 72-hour incubation, indels were detected by next generation sequencing (NGS) of PCR amplicons at the targeted loci. Indel percentage was calculated as the fraction of sequencing reads containing insertions or deletions relative to an unedited reference sequence. The effector proteins Cas9 and Cas12a were used as a positive control. The results are summarized in TABLE 31. An analysis of the results indicates that the effector protein CasM.265466 mRNA and the guides targeting CIITA gene can be used for editing the gene.

TABLE 31
Exemplary modified guides for CIITA editing in T cells

| Effector | | gRNA | | | |
|---|---|---|---|---|---|
| Protein | 5′ | SEQ | | | % |
| SEQ ID | PAM | ID | | Target | Indels |
| NO: | Sec | NO: | RNA Modification | Gene | (3 days) |
| 1 | TGT | 2485 | mA*mC*mA*GCUUAUUUGGAAGCUGA | CIITA | •• |
| | G | | AAUGUGAGGUUUAUAACACUCACAAG | exon: #1 | |
| | | | AAUCCUGAAAAAGGAUGCCAAACUGC | | |
| | | | UUCUGAGCUGGGCAmU*mC*mC | | |
| 1 | TCT | 2486 | mA*mC*mA*GCUUAUUUGGAAGCUGA | CIITA | •• |
| | G | | AAUGUGAGGUUUAUAACACUCACAAG | exon: #1 | |
| | | | AAUCCUGAAAAAGGAUGCCAAACAGC | | |
| | | | UGGGCAUCCGAAGGmC*mA*mU | | |
| 1 | TGT | 2487 | mA*mC*mA*GCUUAUUUGGAAGCUGA | CIITA | • |
| | G | | AAUGUGAGGUUUAUAACACUCACAAG | exon: #1 | |
| | | | AAUCCUGAAAAAGGAUGCCAAACCUU | | |
| | | | CUGAGCUGGGCAUCmC*mG*mA | | |
| 1 | TGT | 2488 | mA*mC*mA*GCUUAUUUGGAAGCUGA | CIITA | ••• |
| | A | | AAUGUGAGGUUUAUAACACUCACAAG | exon: #1 | |
| | | | AAUCCUGAAAAAGGAUGCCAAACGGA | | |
| | | | AUCCCAGCCAGGCAmG*mC*mA | | |
| 1 | TGT | 2489 | mA*mC*mA*GCUUAUUUGGAAGCUGA | CIITA | ••• |
| | G | | AAUGUGAGGUUUAUAACACUCACAAG | exon: #1 | |
| | | | AAUCCUGAAAAAGGAUGCCAAACUAG | | |
| | | | GAAUCCCAGCCAGGmC*mA*mG | | |
| 1 | TCT | 2490 | mA*mC*mA*GCUUAUUUGGAAGCUGA | CIITA | ••• |
| | G | | AAUGUGAGGUUUAUAACACUCACAAG | exon: #1 | |
| | | | AAUCCUGAAAAAGGAUGCCAAACGCA | | |
| | | | GCCCCUCCUCGUGCmC*mC*mU | | |
| 1 | TCT | 2491 | mA*mC*mA*GCUUAUUUGGAAGCUGA | CIITA | •• |
| | G | | AAUGUGAGGUUUAUAACACUCACAAG | exon: #1 | |
| | | | AAUCCUGAAAAAGGAUGCCAAACACA | | |
| | | | GGUAGGACCCAGCAmG*mG*mG | | |
| 1 | TCT | 2492 | mA*mC*mA*GCUUAUUUGGAAGCUGA | CIITA | •• |
| | A | | AAUGUGAGGUUUAUAACACUCACAAG | exon: #2 | |
| | | | AAUCCUGAAAAAGGAUGCCAAACUGA | | |
| | | | CCAGAUGGACCUGGmC*mU*mG | | |
| 1 | TCT | 2493 | mA*mC*mA*GCUUAUUUGGAAGCUGA | CIITA | • |
| | A | | AAUGUGAGGUUUAUAACACUCACAAG | exon: #2 | |
| | | | AAUCCUGAAAAAGGAUGCCAAACCCA | | |
| | | | CUUCUAUGACCAGAmU*mG*mG | | |
| 1 | TAT | 2494 | mA*mC*mA*GCUUAUUUGGAAGCUGA | CIITA | |
| | G | | AAUGUGAGGUUUAUAACACUCACAAG | exon: #2 | • |
| | | | AAUCCUGAAAAAGGAUGCCAAACACC | | |
| | | | AGAUGGACCUGGCUmG*mG*mA | | |
| 1 | TGT | 2495 | mA*mC*mA*GCUUAUUUGGAAGCUGA | CIITA | • |
| | G | | AAUGUGAGGUUUAUAACACUCACAAG | exon: #2 | |
| | | | AAUCCUGAAAAAGGAUGCCAAACCCA | | |
| | | | CCAUGGAGUUGGGGmC*mC*mC | | |
| 1 | TGT | 2496 | mA*mC*mA*GCUUAUUUGGAAGCUGA | CIITA | • |
| | G | | AAUGUGAGGUUUAUAACACUCACAAG | exon: #2 | |
| | | | AAUCCUGAAAAAGGAUGCCAAACCCU | | |
| | | | CUACCACUUCUAUGmA*mC*mC | | |
| 1 | TCT | 2497 | mA*mC*mA*GCUUAUUUGGAAGCUGA | CIITA | •• |
| | A | | AAUGUGAGGUUUAUAACACUCACAAG | exon: #2 | |
| | | | AAUCCUGAAAAAGGAUGCCAAACGGG | | |
| | | | GCCCCAACUCCAUGmG*mU*mG | | |
| 1 | TCT | 2498 | mA*mC*mA*GCUUAUUUGGAAGCUGA | CIITA | • |
| | G | | AAUGUGAGGUUUAUAACACUCACAAG | exon: #2 | |
| | | | AAUCCUGAAAAAGGAUGCCAAACGUC | | |
| | | | AUAGAAGUGGUAGAmG*mG*mC | | |
| 1 | TGT | 2499 | mA*mC*mA*GCUUAUUUGGAAGCUGA | CIITA | •• |
| | G | | AAUGUGAGGUUUAUAACACUCACAAG | exon: #3 | |
| | | | AAUCCUGAAAAAGGAUGCCAAACACA | | |
| | | | UGGAAGGUGAUGAAmG*mA*mG | | |
| 1 | TGT | 2500 | mA*mC*mA*GCUUAUUUGGAAGCUGA | CIITA | •• |
| | G | | AAUGUGAGGUUUAUAACACUCACAAG | exon: #3 | |
| | | | AAUCCUGAAAAAGGAUGCCAAACUGA | | |
| | | | CAUGGAAGGUGAUGmA*mA*mG | | |
| 1 | TAT | 2501 | mA*mC*mA*GCUUAUUUGGAAGCUGA | CIITA | N/A |
| | G | | AAUGUGAGGUUUAUAACACUCACAAG | exon: #4 | |
| | | | AAUCCUGAAAAAGGAUGCCAAACUCU | | |
| | | | UCCAGGACUCCCAGmC*mU*mG | | |

RNA Modification: “*” represents a phosphorothioate bond between the nucleotides, “m” denotes a 2′-OMe modification.
Magnitude of data: “•••” represents a value >60, “••” represents a value between ≤60 and ≥30, “•” represents a value <30.

### Example 19. Gene Editing of B2M, TRAC or CIITA

Guides targeting B2M, TRAC, or CIITA gene are tested with Cas 265466 (SEQ ID NO: 2435) for the ability to produce indels in eukaryotic cells. Briefly, eukaryotic cells are delivered with a combination of mRNA or gene encoding Cas 265466 and a gRNAs or a nucleic acid encoding the gRNAs, wherein the gRNA comprises a handle sequence and any one of the spacer sequence recited in TABLE 32, TABLE 33, and TABLE 34. The handle sequence comprises a nucleotide sequence of ACAGCUUAUUUGGAAGCUGAAAUGUGAGGUUUAUAACACUCACAAGAAUCCUGAAAAA GGAUGCCAAAC (SEQ ID NO: 2522) or mA*mC*mA*GCUUAUUUGGAAGCUGAAAUGUGAGGUUUAUAACACUCACAAGAAUCCUG AAAAAGGAUGCCAAAC (SEQ ID NO: 2523). The CAS 265466 protein (SEQ ID NO: 2435) and the gRNA targeting B2M, TRAC, or CIITA gene forms an RNP complex that recognizes a specific 5′ PAM sequence as identified in TABLE 32, TABLE 33, and TABLE 34.

TABLE 32
CasM.265466 paired with various gRNA
comprising spacer sequences targeting
B2M gene

| Effector | | | |
|---|---|---|---|
| Protein | | 5′ | |
| SEQ ID | | PAM | Target |
| NO | Spacer SEQ ID NO | Seq | Gene |
| 2435 | 1626, 1633, 1634, 1635, 1638, | TGTG | B2M |
| | 1647, 1673, 1674, 1683 | | |
| 2435 | 1627, 1636, 1640, 1641, 1644, | TCTG | B2M |
| | 1649, 1665, 1672, 1675 | | |
| 2435 | 1639, 1628, 1659, 1663, 1677, | TGTA | B2M |
| | 1686 | | |
| 2435 | 1629, 1645, 1654, 1676, 1678, | TCTA | B2M |
| | 1691 | | |
| 2435 | 1630, 1651, 1652, 1658, 1661, | TTTA | B2M |
| | 1669, 1670, 1681, 1682, 1684 | | |
| 2435 | 1632, 1631, 1642, 1657, 1680, | TATG | B2M |
| | 1687 | | |
| 2435 | 1375, 1637, 1643, 1646, 1648, | TTTG | B2M |
| | 1650, 1653, 1655, 1662, 1667, | | |
| | 1685, 1689, 1692 | | |
| 2435 | 1656, 1660, 1664, 1666, 1668, | TATA | B2M |
| | 1671, 1679, 1688, 1690, 1693, | | |
| | 1694 | | |

TABLE 33
CasM.265466 paired with various gRNA
comprising spacer sequences targeting
TRAC gene

| Effector | | | |
|---|---|---|---|
| Protein | | 5′ | |
| SEQ ID | | PAM | Target |
| NO | Spacer SEQ ID NO | Seq | Gene |
| 2435 | 1962, 1966, 1967, 1974, 1977, | TGTG | TRAC |
| | 1983, 1989, 1992, 1995, 1997, | | |
| | 2000, 2005, 2016, 2017 | | |
| 2435 | 1963, 1968, 1979, 2008 | TCTA | TRAC |
| 2435 | 1964, 1970, 1980, 1984, 1986, | TTTG | TRAC |
| | 1987, 1988, 1991, 2014, 2019 | | |
| 2435 | 1965, 1969, 1973, 1976, 1982, | TCTG | TRAC |
| | 1990, 1993, 1996, 1998, 1999, | | |
| | 2003, 2009, 2011, 2012, 2013, | | |
| | 2015, 2018 | | |
| 2435 | 1971, 1981 | TATA | TRAC |
| 2435 | 1972, 1975, 2001, 2002, 2006 | TGTA | TRAC |
| 2435 | 1978, 2004, 2010 | TATG | TRAC |
| 2435 | 1985, 1994, 2007 | TTTA | TRAC |

TABLE 34
CasM.265466 paired with various gRNA
comprising spacer sequences targeting
CIITA gene

| Effector | | | |
|---|---|---|---|
| Protein | | 5′ | |
| SEQ ID | | PAM | Target |
| NO | Spacer SEQ ID NO | Seq | Gene |
| 2435 | 1754, 1756, 1758, 1764, | TGTG | CIITA |
| | 1765, 1768, 1769 | | |
| 2435 | 1755, 1759, 1760, 1767 | TCTG | CIITA |
| 2435 | 1757 | TGTA | CIITA |
| 2435 | 1761, 1762, 1766 | TCTA | CIITA |
| 2435 | 1763, 1770 | TATG | CIITA |

The cells are incubated for about 48 hours to 96 hours to allow indel formation. Indels are detected by next generation sequencing of PCR amplicons at the targeted loci and indel percentage is calculated as the fraction of sequencing reads containing insertions or deletions relative to an unedited reference sequence.

### Example 20. Determining Ability of CasM.265466 to Generate Indels in T Cells

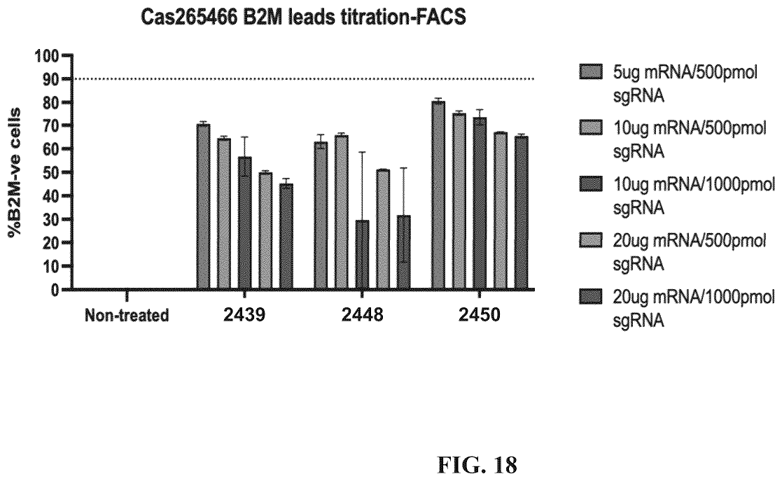

A dose titration for CasM.265466 mRNA and sgRNAs was performed to improve indel formation in T cells. Briefly, sgRNAs having a handle sequence of SEQ ID NO: 2523 and a spacer sequence of each of SEQ ID NO: 2439, 2448, and 2450 were dose titrated with mRNA encoding Cas 265466 (SEQ ID NO: 2435) to determine gene editing efficiency at different doses following a similar protocol as described in Example 16 but with different amounts of sgRNA and Cas 265466 effector mRNA. Specifically, sgRNAs having the spacer sequences of each of SEQ ID NO: 2439, 2448, and 2450 were electroporated with Cas 265466 mRNA in the following conditions: 1) 5 μg Cas 265466 mRNA and 500 pmol sgRNA; 2) 10 μg Cas 265466 mRNA and 500 pmol sgRNA; 3) 10 μg Cas 265466 mRNA and 1000 pmol sgRNA; 4) 20 μg Cas 265466 mRNA and 500 pmol sgRNA; and 5) 20 μg Cas 265466 mRNA and 1000 pmol sgRNA. The T cells were electroporated with the combination and incubated for about 72 hours. Indels were detected by flow cytometry (FACS) using B2M antibody and next generation sequencing (NGS) of PCR amplicons at the targeted loci 3 days post electroporation. The results of the FACS analysis are shown in FIG. 18. The Y-axis shows the percent B2M negative cells. The X-axis shows the different sgRNAs. The conditions indicated above are presented left to right on the graphs for each sgRNA. Indel percentage was calculated as the fraction of sequencing reads containing insertions or deletions relative to an unedited reference sequence which are summarized in TABLE 35. An analysis of results demonstrate successful editing of B2M gene in the T cells by CasM.265466 and sgRNA at a range of concentration ratios.

TABLE 35
Indel Formation using CasM.265466
mRNA paired with various sgRNA in T cells

| | mRNA | sgRNA | Spacer SEQ | % |
|---|---|---|---|---|
| No. | Dose (ng) | Dose (ng) | ID NO: | INDELS |
| 1 | 5 | 500 | 2439 | •• |
| | | | 2448 | •• |
| | | | 2450 | ••• |
| 2 | 10 | 500 | 2439 | •• |
| | | | 2448 | •• |
| | | | 2450 | ••• |
| 3 | 10 | 1000 | 2439 | •• |
| | | | 2448 | •• |
| | | | 2450 | ••• |
| 4 | 20 | 500 | 2439 | •• |
| | | | 2448 | •• |
| | | | 2450 | ••• |
| 5 | 20 | 1000 | 2439 | •• |
| | | | 2448 | •• |
| | | | 2450 | •• |

% Indels represents 3 day post editing NGS Indel percentage data. Magnitude of Indel percentage data: “•••” represents a value over 80, “••” represents a value under 80 but over 50, “•” represents a value under 50.

### Example 21. Dose titration for TRAC editing

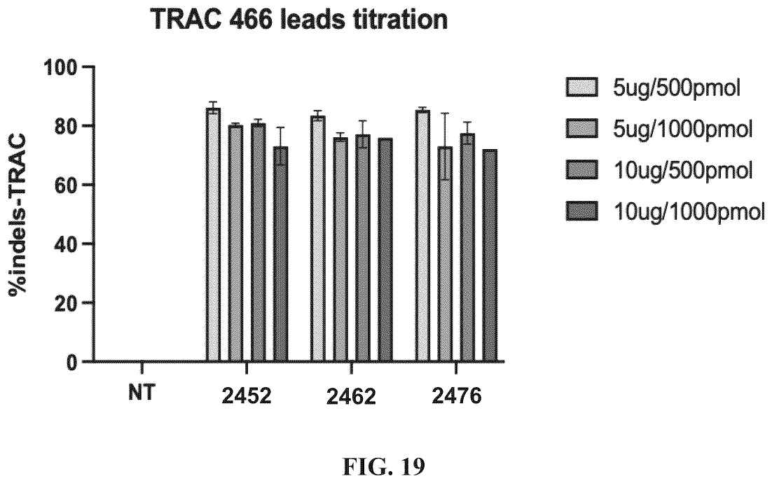

A dose titration for CasM.265466 mRNA and sgRNAs was performed to improve indel formation in T cells. Briefly, sgRNAs having a handle sequence of SEQ ID NO: 2523 and a spacer sequence of each of SEQ ID NO: 2452, 2462 and 2476 were dose titrated with mRNA encoding Cas 265466 (SEQ ID NO: 2435) to determine gene editing efficiency at different doses following a similar protocol as described in Example 17 but with different amounts of sgRNA and Cas 265466 effector mRNA. Specifically, sgRNAs having each of spacer sequences of SEQ ID NO: 2452, 2462 and 2476 were electroporated with Cas 265466 mRNA in the following conditions: 1) 5 μg Cas 265466 mRNA and 500 pmol sgRNA; 2) 5 μg Cas 265466 mRNA and 1000 pmol sgRNA; 3) 10 μg Cas 265466 mRNA and 500 pmol sgRNA; and 4) 10 μg Cas 265466 mRNA and 1000 pmol sgRNA. The results of the sequence analysis are shown in FIG. 10. The Y-axis shows the percent indels in the TRAC gene. The X-axis shows the different sgRNAs, and NT indicates non-treated. The conditions indicated above are presented left to right on the graphs for each sgRNA. The sequencing graph shown in FIG. 19, shows of the percent indels of TRAC. An analysis of FIG. 19 indicates that the 5 μg Cas 265466 mRNA in combination with 500 pmol sgRNA having the spacer sequences of each of SEQ ID NO: 2452, 2462 and 2476 had about 80% indels. The analysis further suggests that the 5 μg Cas 265466 mRNA and 500 pmol sgRNA condition produced the highest amount of editing.

### Example 22. Dose Titration for CIITA Editing

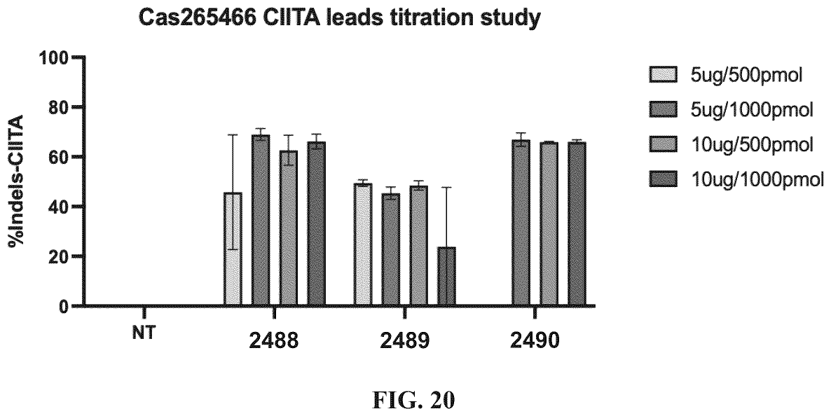

A dose titration for CasM.265466 mRNA and sgRNAs was performed to improve indel formation in T cells. Briefly, sgRNAs having a handle sequence of SEQ ID NO: 2523 and a spacer sequence of each of SEQ ID NO: 2488, 2489 and 2490 were dose titrated with mRNA encoding Cas 265466 (SEQ ID NO: 2435) to determine gene editing efficiency at different doses following a similar protocol as described in Example 18 but with different amounts of sgRNA and Cas 265466 effector mRNA. Briefly, sgRNAs having each of spacer sequences of SEQ ID NO: 2488, 2489 and 2490 were electroporated with Cas 265466 mRNA in the following conditions: 1) 5 μg Cas 265466 mRNA and 500 pmol sgRNA; 2) 5 μg Cas 265466 mRNA and 1000 pmol sgRNA; 3) 10 μg Cas 265466 mRNA and 500 pmol sgRNA; and 4) 10 μg Cas 265466 mRNA and 1000 pmol sgRNA. The results of the sequence analysis are shown in FIG. 20. The Y-axis shows the percent indels in the CIITA gene. The X-axis shows the different sgRNAs, and NT indicates non-treated. The conditions indicated above are presented left to right on the graphs for each sgRNA. The sequencing graph shown in FIG. 20, shows of the percent indels of CIITA. An analysis of FIG. 20 indicates that The results show the: 5 μg Cas 265466 mRNA and 1000 pmol sgRNA; 10 μg Cas 265466 mRNA and 500 pmol sgRNA; and 10 μg Cas 265466 mRNA and 1000 pmol sgRNA conditions produced the highest amount of editing.

### Example 23. B2M editing in NK cells

B2M guides targeting exon 2 of B2M were tested with Cas 265466 for the ability to produce indels in primary NK Cells. Briefly, the NK cells were electroporated with a mixture of mRNA encoding the Cas nuclease (SEQ ID NO: 2435) and gRNA of different guides (SEQ ID NO: 2439 and 2448) were mixed and then electroporated. 5 μg of Cas 265466 was added for the assay and 500 pmol of gRNA was added for the assay. Different electroporation conditions were used to determine the highest efficiency for NK cell electroporation and are described below. Individual gRNA were used with the effector proteins. After electroporation, the cells were incubated at 37° C. and 5% CO2 for 72 hours.

After the 72-hour incubation, cells were analyzed for indels in B2M. FIG. 21 shows sequencing data at Day 3 showing the percent indels in B2M for the Cas 265466 and gRNA with different electroporation conditions. The Y-axis shows the percent of indels. The X-axis shows the different gRNAs, and NT indicates non-treated. The gRNAs and Cas 265466 were electroporated with different conditions. Briefly, the sgRNAs were electroporated with Cas 265466 mRNA in the following conditions: 1) 1600 Volts (V) for 20 milliseconds (ms) with 1 pulse; 2) 1700 V for 20 ms with 1 pulse; 3) 1300 V for 30 ms with 1 pulse; 4) 1300 V for 30 ms with 2 pulses; and 5) 1850 V for 10 ms with 2 pulses. The conditions indicated above are presented left to right on the graph for each gRNA. The conditions of 1) 1600 V for 20 ms with 1 pulse and 5) 1850 V for 10 ms with 2 pulses produced the highest percentage of indels in guides having nucleic acid of SEQ ID NO: 2439 and 2448. The condition of 1) 1600 V for 20 ms with 1 pulse produced about 20-30% indels in B2M of the primary NK cells with either guides having nucleic acid of SEQ ID NO: 2439 or 2448, and the condition of 5) 1850 V for 10 ms with 2 pulses produced about 20% indels in B2M of the primary NK cells. The results show Cas 265466 with different guide constructs can edit B2M in NK cells.

### Example 24. Gene Editing of Primary T Cells with scAAV Vector Encoding CasM.265466 and Guide RNA

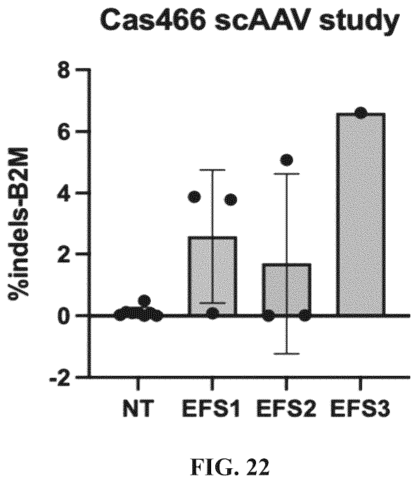

scAAV plasmid constructs were tested for their ability to produce indels in B2M of primary T cells. Briefly, a scAAV plasmid was constructed to contain a transgene between its ITRs, the transgene providing or encoding, in a 5′ to 3′ direction, a U6 promoter, a guide RNA, an EFS promoter, a Cas effector protein, and a SV40 poly A tail. The EFS promoter was EFS1, EFS2, or EFS3, wherein EFS1 promoter construct refers to the scAAV plasmid encoding the guide RNA having SEQ ID NO: 2439, EFS2 promoter construct refers to the scAAV plasmid encoding the guide RNA having SEQ ID NO: 2450, and EFS3 promoter construct refers to the scAAV plasmid encoding the guide RNA having SEQ ID NO: 2448. The Cas effector protein was Cas 265466 (SEQ ID NO: 2435). The guide RNA had a nucleotide sequence that targets B2M gene. The scAAV vector was expressed with supporting plasmids to produce an adeno-associated virus (AAV). Activated primary T cells were transduced with the AAV. DNA was isolated from the infected cells post transduction. An indel in B2M caused by the guide nucleic acid was confirmed by sequencing. The scAAV results are summarized in FIG. 22, which shows the percent indels in B2M on the Y-axis and the different scAAV constructs varying in the EFS promoter on the X-axis. NT on the X-axis indicates non-treated. The EFS3 promoter construct produced the highest percent (6%) of indels in B2M. The results indicate that AAV encoding Cas 265466 and an sgRNA can be used to edit genes in primary T cells.

### Example 25. Gene editing of eukaryotic cells with scAAV vector encoding CasM.19952 and guide RNA

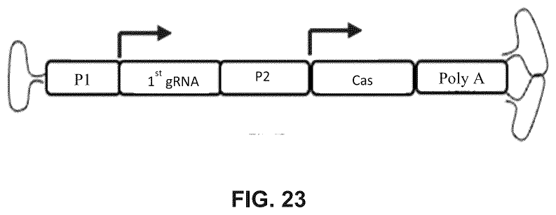

An scAAV vector is constructed to contain a transgene between its ITRs, the transgene providing or encoding, in a 5′ to 3′ direction, a U6 promoter, a guide RNA, an EFS promoter, a Cas effector protein, and a SV40 poly A tail as illustrated in FIG. 23. The Cas effector comprises a sequence of SEQ ID NO: 23. The guide RNA that are used for gene editing includes SEQ ID NOs: 2502-2511. The AAV vector is expressed with supporting plasmids to produce an adeno-associated virus (AAV). Eukaryotic cells are contacted with the AAV for 24 hours. After about 96 hours, post AAV contact, DNA or RNA is isolated from the infected eukaryotic cells. An indel caused by the guide nucleic acid is confirmed by sequencing and/or Q-PCR. TABLE 36 recites amplicons (SEQ ID NOs: 2512-2521) that are sequenced for measuring indel activity with a specific guide RNA.

TABLE 36
Amplicon Sequences used with sgRNA in
Primary T Cells

| sgRNA (SEQ ID NO:) | Amplicon sequence |
|---|---|
| UGGGGCAGUUGGUUGCCC | ACACAGACACCATCAACTGCGACCAGTTC |
| UUAGCCUGAGGCAUUUAU | AGCAGGCTGTTGTGTGACATGGAAGGTGA |
| UGCACUCGGGAAGUACCA | TGAAGAGACCAGGGAGGCTTATGCCAATA |
| UUUCUCAGAAAUGGUACA | TCGGTGAGGAAGCACCTGAGCCCAGAAAA |
| UCCAACGUGAGGAAGCAC | GGACAATCAAGGGCAAGAGTTCTTTGCTG |
| CUGAGCCC (SEQ ID | CCACTTGTCAATATCACCCATTCATCATG |
| NO: 2502) | AGCCACGT (SEQ ID NO: 2512) |
| UGGGGCAGUUGGUUGCCC | TCTAGGGATGGTGGCTTCTGGAAGGCTGA |
| UUAGCCUGAGGCAUUUAU | CCATGCACAGGCCTCCAATCCCTCCCCCT |
| UGCACUCGGGAAGUACCA | GGCCTCTGTTTCCGACAGCTTGTACAATA |
| UUUCUCAGAAAUGGUACA | ACTGCATCTGCGACGTGGGAGCCGAGAGC |
| UCCAACCAGAUGCAGUUA | TTGGCTCGTGTGCTTCCGGACATGGTGTC |
| UUGUACAA (SEQ ID | CCTCCGGGTGATGGAGTGAGTGTGGGAGT |
| NO: 2503) | CTGGGCGGTGGGTGGCTCAGCCCGGGGTG |
| | GGAGACACTGAAGTCTCTCCCTGGTGTC |
| | (SEQ ID NO: 2513) |
| UGGGGCAGUUGGUUGCCC | GGATGGGAAGGGTCAGATGGCCCCAGGAC |
| UUAGCCUGAGGCAUUUAU | GCTAGCTGATGGCCCCCATCTGATTCCAC |
| UGCACUCGGGAAGUACCA | CTGCAGCCTGGATGCGCTGAGTGAGAACA |
| UUUCUCAGAAAUGGUACA | AGATCGGGGACGAGGGTGTCTCGCAGCTC |
| UCCAACCAGCUCUCAGCC | TCAGCCACCTTCCCCCAGCTGAAGTCCTT |
| ACCUUCCC (SEQ ID | GGAAACCCTCAAGTGAGTGAGCTGGGCCT |
| NO: 2504) | GCCCTTCCTGCTGAATCGGGCCCCCAAAG |
| | TCCGGCTGACTTTTTCAAAATTAATTTAA |
| | ATTTGTTTTTTTAGACAAGGGCTCGCTG |
| | (SEQ ID NO: 2514) |
| UGGGGCAGUUGGUUGCCC | GCCCAAGAACTAGGAGGTCTGGGGTGGGA |
| UUAGCCUGAGGCAUUUAU | GAGTCAGCCTGCTCTGGATGCTGAAAGAA |
| UGCACUCGGGAAGUACCA | TGTCTGTTTTTCCTTTTAGAAAGTTCCTG |
| UUUCUCAGAAAUGGUACA | TGATGTCAAGCTGGTCGAGAAAAGCTTTG |
| UCCAACACCAGCUUGACA | AAACAGGTAAGACAGGGGTCTAGCCTGGG |
| UCACAGGA (SEQ ID | TTTGCACAGGATTGCGGAAGTGATGAACC |
| NO: 2505) | CGCAATAACCCTGCCTGGATGAGGGAGTG |
| | GGAAGAAATTAGTAGATGTGGGAATGAAT |
| | GATGAGGAATGGAAACAGCGGTT (SEQ |
| | ID NO: 2515) |
| UGGGGCAGUUGGUUGCCC | AGGGGATATGCACAGAAGCTGCAAGGGAC |
| UUAGCCUGAGGCAUUUAU | AGGAGGTGCAGGAGCTGCAGGCCTCCCCC |
| UGCACUCGGGAAGUACCA | ACCCAGCCTGCTCTGCCTTGGGGAAAACC |
| UUUCUCAGAAAUGGUACA | GTGGGTGTGTCCTGCAGGCCATGCAGGCC |
| UCCAACGAACCCAAUCAC | TGGGACATGCAAGCCCATAACCGCTGTGG |
| UGACAGGU (SEQ ID | CCTCTTGGTTTTACAGATACGAACCTAAA |
| NO: 2506) | CTTTCAAAACCTGTCAGTGATTGGGTTCC |
| | GAATCCTCCTCCTGAAAGTGGCCGGGTTT |
| | AATCTGCTCATGACGCTGC (SEQ ID |
| | NO: 2516) |
| UGGGGCAGUUGGUUGCCC | AGGGGATATGCACAGAAGCTGCAAGGGAC |
| UUAGCCUGAGGCAUUUAU | AGGAGGTGCAGGAGCTGCAGGCCTCCCCC |
| UGCACUCGGGAAGUACCA | ACCCAGCCTGCTCTGCCTTGGGGAAAACC |
| UUUCUCAGAAAUGGUACA | GTGGGTGTGTCCTGCAGGCCATGCAGGCC |
| UCCAACUAUCUGUAAAAC | TGGGACATGCAAGCCCATAACCGCTGTGG |
| CAAGAGGC (SEQ ID | CCTCTTGGTTTTACAGATACGAACCTAAA |
| NO: 2507) | CTTTCAAAACCTGTCAGTGATTGGGTTCC |
| | GAATCCTCCTCCTGAAAGTGGCCGGGTTT |
| | AATCTGCTCATGACGCTGC (SEQ ID |
| | NO: 2517) |
| UGGGGCAGUUGGUUGCCC | AATATAAGTGGAGGCGTCGCGCTGGCGGG |
| UUAGCCUGAGGCAUUUAU | CATTCCTGAAGCTGACAGCATTCGGGCCG |
| UGCACUCGGGAAGUACCA | AGATGTCTCGCTCCGTGGCCTTAGCTGTG |
| UUUCUCAGAAAUGGUACA | CTCGCGCTACTCTCTCTTTCTGGCCTGGA |
| UCCAACCGCUACUCUCUC | GGCTATCCAGCGTGAGTCTCTCCTACCCT |
| UUUCUGGC (SEQ ID | CCCGCTCTGGTCCTTCCTCTCCCGCTCTG |
| NO: 2508) | CACCCTCTGTGGCCCTCGCTGTGCTCTCT |
| | CGCTCCGTGACTTCCCTTCTCC (SEQ |
| | ID NO: 2518) |
| UGGGGCAGUUGGUUGCCC | CCCAAGTGAAATACCCTGGCAATATTAAT |
| UUAGCCUGAGGCAUUUAU | GTGTCTTTTCCCGATATTCCTCAGGTACT |
| UGCACUCGGGAAGUACCA | CCAAAGATTCAGGTTTACTCACGTCATCC |
| UUUCUCAGAAAUGGUACA | AGCAGAGAATGGAAAGTCAAATTTCCTGA |
| UCCAACGAUGGAUGAAAC | ATTGCTATGTGTCTGGGTTTCATCCATCC |
| CCAGACAC (SEQ ID | GACATTGAAGTTGACTTACTGAAGAATGG |
| NO: 2509) | AGAGAGAATTGAAAAAGTGGAGCATTCAG |
| | ACTTGTCTTTCAGCAAGGACTGGTCTTTC |
| | TATCTCTTGTACTACACTGAATTCACCCC |
| | CACTG (SEQ ID NO: 2519) |
| UGGGGCAGUUGGUUGCCC | AGCCTATTCTGCCAGCCTTATTTCTAACC |
| UUAGCCUGAGGCAUUUAU | ATTTTAGACATTTGTTAGTACATGGTATT |
| UGCACUCGGGAAGUACCA | TTAAAAGTAAAACTTAATGTCTTCCTTTT |
| UUUCUCAGAAAUGGUACA | TTTTCTCCACTGTCTTTTTCATAGATCGA |
| UCCAACAUCUAUGAAAAA | GACATGTAAGCAGCATCATGGAGGTAAGT |
| GACAGUGG (SEQ ID | TTTTGACCTTGAGAAAATGTTTTTGTTTC |
| NO: 2510) | ACTGTCCTGAGGACTATTTATAGACAGCT |
| | CTAACATGATAACCCTCACTATGTGGAGA |
| | ACAT (SEQ ID NO: 2520) |
| UGGGGCAGUUGGUUGCCC | CCTCTCTCTAACCTGGCACTGCGTCGCTG |
| UUAGCCUGAGGCAUUUAU | GCTTGGAGACAGGTGACGGTCCCTGCGGG |
| UGCACUCGGGAAGUACCA | CCTTGTCCTGATTGGCTGGGCACGCGTTT |
| UUUCUCAGAAAUGGUACA | AATATAAGTGGAGGCGTCGCGCTGGCGGG |
| UCCAACCUCCGUGGCCUU | CATTCCTGAAGCTGACAGCATTCGGGCCG |
| AGCUGUGC (SEQ ID | AGATGTCTCGCTCCGTGGCCTTAGCTGTG |
| NO: 2511) | CTCGCGCTACTCTCTCTTTCTGGCCTGGA |
| | GGCTATCCAGCGTGAGTCTCTCCTACCCT |
| | C (SEQ ID NO: 2521) |

### Example 26. Gene Editing of Primary T Cells with scAAV Vector Encoding CasM.19952 and Guide RNA

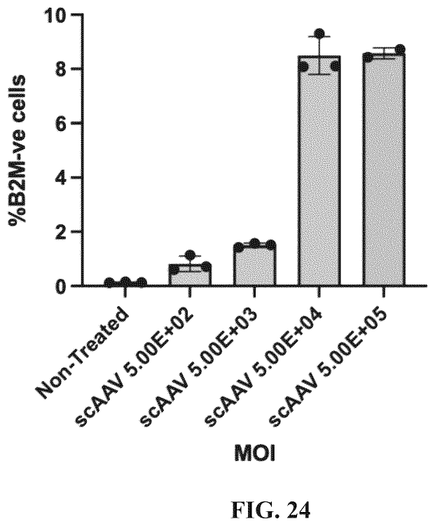

A dose response experiment for scAAV plasmid for testing its ability to produce indels in primary T cells was conducted. Briefly, a scAAV plasmid was constructed to contain a transgene between its ITRs, the transgene providing or encoding, in a 5′ to 3′ direction, a U6 promoter, a guide RNA, an EFS promoter, a Cas effector protein, and a SV40 poly A tail. The Cas effector protein was CasM.19952 (SEQ ID NO: 23). The guide RNA had a nucleotide sequence of SEQ ID NO: 364. The scAAV vector was expressed with supporting plasmids to produce an adeno-associated virus (AAV). Activated primary T cells were transduced with the AAV at various concentrations (0, 5e+02, 5e+03, 5e+04, and 5e+05 GC/cell). About 96 hours post transduction, DNA or RNA was isolated from the infected cells. An indel caused by the guide nucleic acid was confirmed by sequencing and/or Q-PCR using amplicon SEQ ID NO: 472. Results of the dose response experiment are summarized in FIG. 24. An analysis of FIG. 24 indicates that AAV can be used to edit genes in primary T cells.

While various embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 27. CasΦ.12 L26R mediated GFP integration in T cells

This example demonstrates the potential for generation of CAR T cells by integration of an exemplary GFP marker into the TRAC locus of T cells using RNP complexes of CasΦ.12 L26R having an amino acid sequence of SEQ ID NO: 2592, and a TRAC specific guide RNA having a sequence of mC*mU*mU*UCAAGACUAAUAGAUUGCUCCUUACGAGGAGACGAGUCUCUCAGCUG GmU* mA*mC (SEQ ID NO: 2593). Briefly, 2.5×10⁶ activated T cells were electroporated with a mixture of an mRNA encoding the CasΦ.12 L26R (10 μg) and an mRNA encoding the TRAC specific guide RNA (500 μmol). The transfected cells were divided into two portions. The first portion of the transfected cells were incubated at 37° C. and 5% CO₂ to allow for indel formation. The other portion was incubated at 37° C. and 5% CO₂ for 2 hours before AAV transduction. For the AAV transduction, AAV6 particles containing a donor nucleotide sequence encoding the GFP marker was added at 5×10⁵ MOI of the electroporated T cells for 24 hours. The transduced cells were washed of AAV6 particles and further incubated at 37° C. and 5% CO₂ to allow for knock in of the GFP marker. After 6 days post-transfection, the cells were processed by fluorescence-activated cell sorting (FACS) analysis and next generation sequencing (NGS) analysis. Indel percentage was calculated as the fraction of sequencing reads containing insertions or deletions relative to an unedited reference sequence. For negative control, AAV6 particles containing a donor nucleotide sequence encoding the GFP marker was used with activated naïve T cells.

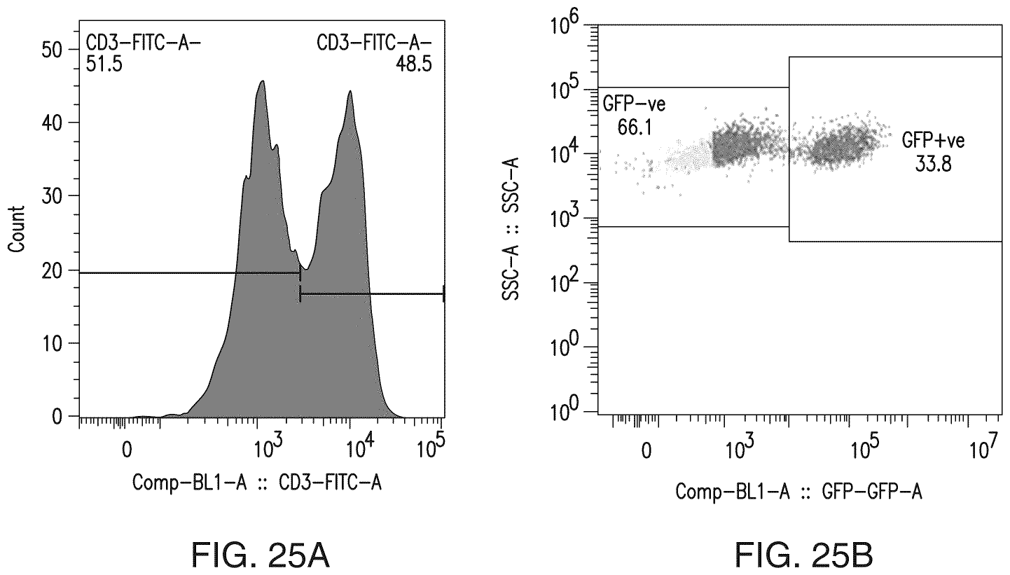

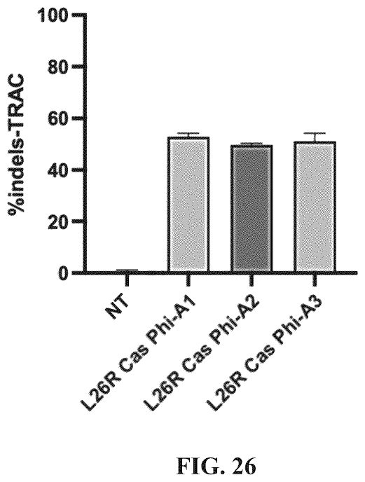

Results of the TRAC gene knockout is shown in FIG. 25A, and results of GFP knock-in into the TRAC locus are shown in FIG. 25B. An analysis of FIGS. 25A-25B suggests that a donor nucleic acid can be integrated into the TRAC locus using the method described herein. The results were further confirmed by % indel analysis (FIG. 26).

### Example 28. CasΦ.12 L26R Mediated CD19-CAR Integration in T Cells

This example demonstrates the generation of CAR T cells by integration of a CD19-CAR encoding donor nucleic acid into the TRAC locus of T cells using RNP complexes of CasΦ.12 L26R having an amino acid sequence of SEQ ID NO: 2592, and a TRAC specific guide RNA having a sequence of mC*mU*mU*UCAAGACUAAUAGAUUGCUCCUUACGAGGAGACGAGUCUCUCAGCUG GmU* mA*mC (SEQ ID NO: 2593). Briefly, 2.5×10⁶ activated T cells were electroporated with a mixture of an mRNA encoding the CasΦ.12 L26R (10 μg) and an mRNA encoding the TRAC specific guide RNA (500 μmol). The transfected cells were divided into two portions. The first portion of the transfected cells were incubated at 37° C. and 5% CO₂ to allow for indel formation. The other portion was incubated at 37° C. and 5% CO₂ for 2 hours before AAV transduction. For the AAV transduction, AAV6 particles containing a donor nucleotide sequence encoding the CD19-CAR was added at 5×10⁵ MOI of the electroporated T cells for 24 hours. The transduced cells were washed of AAV6 particles and further incubated at 37° C. and 5% CO₂ to allow for knock-in of the CD19-CAR. After 6 days post-transfection, the cells were processed by fluorescence-activated cell sorting (FACS) analysis and next generation sequencing (NGS) analysis. Indel percentage was calculated as the fraction of sequencing reads containing insertions or deletions relative to an unedited reference sequence.

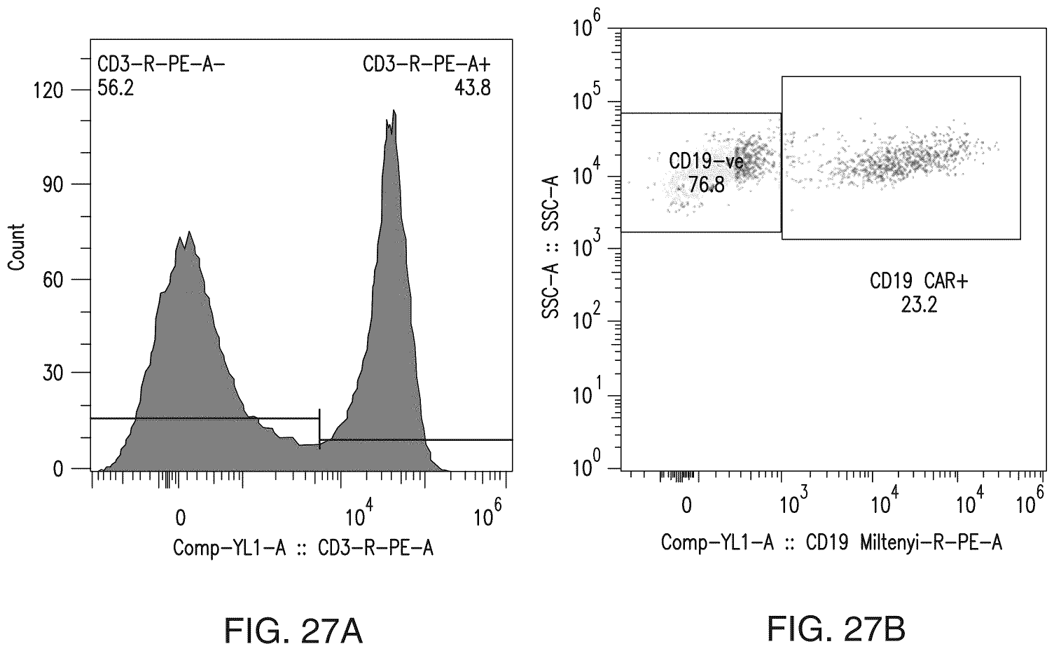

Results of the TRAC gene knockout is shown in FIG. 27A, and results of the CD19-CAR knock-in into the TRAC locus are shown in FIG. 27B. An analysis of FIGS. 27A-27B suggests that a donor nucleic acid can be integrated into the TRAC locus using the method described herein.

### Example 29. CasΦ.12 Mediated Single-Stranded Oligodeoxynucleotides (ssODNs) Integration in T Cells by HDR Pathway

This example demonstrates single-stranded oligodeoxynucleotides (ssODNs) integration in T cells by HDR pathway using an RNP complex of CasΦ.12 having an amino acid sequence of SEQ ID NO: 57, and a guide RNA targeting TRAC gene (AUUGCUCCUUACGAGGAGACGAGUCUCUCAGCUGGUA (SEQ ID NO: 1357)) or B2M gene (AUUGCUCCUUACGAGGAGACGGGCCGAGAUGUCUCGC (SEQ ID NO: 2639)), where the last 3 nucleotides of this gRNA were chemically modified with 2′ 0-Methyl. Briefly, 5×10⁵ activated T cells were electroporated with a mixture of an mRNA encoding the CasΦ.12 (250 μmol), an mRNA encoding the guide RNA (500 μmol), and a donor nucleic acid (150 μmol). 24 donor nucleic acids were designed for knock-in into the TRAC gene, wherein the donor nucleic acids were chemically modified for enhancing HDR. In contrast, 12 donor nucleic acids were designed for knock-in into the B32M gene. TABLE 36 lists sequences of the donor nucleic acids that are tested for this experiment.

TABLE 37
Donor Nucleic Acid Sequences

| Target | SEQ ID | |
|---|---|---|
| Gene | NO: | Sequence |
| TRAC | 2603 | AAAATCGGTGAATAGGCAGACAGACTTGTCACTGGATTTAGCGG |
| | | CCGCGAGTCTCTCAGCTGGTACACGGCAGGGTCAGGGTTCTGG |
| TRAC | 2604 | CAGACAGACTTGTCACTGGATTTAGAGTCTCTCAGCTGGTGCGG |
| | | CCGCACACGGCAGGGTCAGGGTTCTGGATATCTGTGGGACAAGA |
| TRAC | 2605 | CACAGATATCCAGAACCCTGACCCTGCCGTGTACCAGCTGGCGG |
| | | CCGCAGAGACTCTAAATCCAGTGACAAGTCTGTCTGCCTATTCA |
| TRAC | 2606 | AATCGGTGAATAGGCAGACAGACTTGTCACTGGATTTAGAGCGG |
| | | CCGCGTCTCTCAGCTGGTACACGGCAGGGTCAGGGTTCTGGATA |
| TRAC | 2607 | GACAGACTTGTCACTGGATTTAGAGTCTCTCAGCTGGTACGCGG |
| | | CCGCACGGCAGGGTCAGGGTTCTGGATATCTGTGGGACAAGAGG |
| TRAC | 2608 | CCCACAGATATCCAGAACCCTGACCCTGCCGTGTACCAGCGCGG |
| | | CCGCTGAGAGACTCTAAATCCAGTGACAAGTCTGTCTGCCTATT |
| TRAC | 2609 | TCGGTGAATAGGCAGACAGACTTGTCACTGGATTTAGAGTGCGG |
| | | CCGCCTCTCAGCTGGTACACGGCAGGGTCAGGGTTCTGGATATC |
| TRAC | 2610 | CAGACTTGTCACTGGATTTAGAGTCTCTCAGCTGGTACACGCGG |
| | | CCGCGGCAGGGTCAGGGTTCTGGATATCTGTGGGACAAGAGGAT |
| TRAC | 2611 | GTCCCACAGATATCCAGAACCCTGACCCTGCCGTGTACCAGCGG |
| | | CCGCGCTGAGAGACTCTAAATCCAGTGACAAGTCTGTCTGCCTA |
| TRAC | 2612 | GGTGAATAGGCAGACAGACTTGTCACTGGATTTAGAGTCTGCGG |
| | | CCGCCTCAGCTGGTACACGGCAGGGTCAGGGTTCTGGATATCTG |
| TRAC | 2613 | TGTCACTGGATTTAGAGTCTCTCAGCTGGTACACGGCAGGGGGG |
| | | CCGCGTCAGGGTTCTGGATATCTGTGGGACAAGAGGATCAGGGT |
| TRAC | 2614 | TTGTCCCACAGATATCCAGAACCCTGACCCTGCCGTGTACGCGG |
| | | CCGCCAGCTGAGAGACTCTAAATCCAGTGACAAGTCTGTCTGCC |
| TRAC | 2615 | TGAATAGGCAGACAGACTTGTCACTGGATTTAGAGTCTCTGCGG |
| | | CCGCCAGCTGGTACACGGCAGGGTCAGGGTTCTGGATATCTGTG |
| TRAC | 2616 | TCCAGAACCCTGACCCTGCCGTGTACCAGCTGAGAGACTCGCGG |
| | | CCGCTAAATCCAGTGACAAGTCTGTCTGCCTATTCACCGATTTT |
| TRAC | 2617 | TCTTGTCCCACAGATATCCAGAACCCTGACCCTGCCGTGTGCGG |
| | | CCGCACCAGCTGAGAGACTCTAAATCCAGTGACAAGTCTGTCTG |
| TRAC | 2618 | AATAGGCAGACAGACTTGTCACTGGATTTAGAGTCTCTCAGCGG |
| | | CCGCGCTGGTACACGGCAGGGTCAGGGTTCTGGATATCTGTGGG |
| TRAC | 2619 | TATCCAGAACCCTGACCCTGCCGTGTACCAGCTGAGAGACGCGG |
| | | CCGCTCTAAATCCAGTGACAAGTCTGTCTGCCTATTCACCGATT |
| TRAC | 2620 | CCTCTTGTCCCACAGATATCCAGAACCCTGACCCTGCCGTGCGG |
| | | CCGCGTACCAGCTGAGAGACTCTAAATCCAGTGACAAGTCTGTC |
| TRAC | 2621 | TAGGCAGACAGACTTGTCACTGGATTTAGAGTCTCTCAGCGCGG |
| | | CCGCTGGTACACGGCAGGGTCAGGGTTCTGGATATCTGTGGGAC |
| TRAC | 2622 | GATATCCAGAACCCTGACCCTGCCGTGTACCAGCTGAGAGGCGG |
| | | CCGCACTCTAAATCCAGTGACAAGTCTGTCTGCCTATTCACCGA |
| TRAC | 2623 | ATCCTCTTGTCCCACAGATATCCAGAACCCTGACCCTGCCGCGG |
| | | CCGCGTGTACCAGCTGAGAGACTCTAAATCCAGTGACAAGTCTG |
| TRAC | 2624 | GGCAGACAGACTTGTCACTGGATTTAGAGTCTCTCAGCTGGCGG |
| | | CCGCGTACACGGCAGGGTCAGGGTTCTGGATATCTGTGGGACAA |
| TRAC | 2625 | CAGATATCCAGAACCCTGACCCTGCCGTGTACCAGCTGAGGCGG |
| | | CCGCAGACTCTAAATCCAGTGACAAGTCTGTCTGCCTATTCACC |
| TRAC | 2626 | ACCCTGATCCTCTTGTCCCACAGATATCCAGAACCCTGACGCGG |
| | | CCGCCCTGCCGTGTACCAGCTGAGAGACTCTAAATCCAGTGACA |
| B2M | 2627 | GGCGTCGCGCTGGCGGGCATTCCTGAAGCTGACAGCATTCgcgg |
| | | ccgcGGGCCGAGATGTCTCGCTCCGTGGCCTTAGCTGTGCTCGC |
| B2M | 2628 | CGTCGCGCTGGCGGGCATTCCTGAAGCTGACAGCATTCGGgcgg |
| | | ccgcGCCGAGATGTCTCGCTCCGTGGCCTTAGCTGTGCTCGCGC |
| B2M | 2629 | TCGCGCTGGCGGGCATTCCTGAAGCTGACAGCATTCGGGCgcgg |
| | | ccgcCGAGATGTCTCGCTCCGTGGCCTTAGCTGTGCTCGCGCTA |
| B2M | 2630 | GCGCTGGCGGGCATTCCTGAAGCTGACAGCATTCGGGCCGgcgg |
| | | ccgcAGATGTCTCGCTCCGTGGCCTTAGCTGTGCTCGCGCTACT |
| B2M | 2631 | GCTGGCGGGCATTCCTGAAGCTGACAGCATTCGGGCCGAGgcgg |
| | | ccgcATGTCTCGCTCCGTGGCCTTAGCTGTGCTCGCGCTACTCT |
| B2M | 2632 | TGGGGGCATTCCTGAAGCTGACAGCATTCGGGCCGAGATgcggc |
| | | cgcGTCTCGCTCCGTGGCCTTAGCTGTGCTCGCGCTACTCTCT |
| B2M | 2633 | GCGGGCATTCCTGAAGCTGACAGCATTCGGGCCGAGATGTgcgg |
| | | ccgcCTCGCTCCGTGGCCTTAGCTGTGCTCGCGCTACTCTCTCT |
| B2M | 2634 | GGGCATTCCTGAAGCTGACAGCATTCGGGCCGAGATGTCTgcgg |
| | | ccgcCGCTCCGTGGCCTTAGCTGTGCTCGCGCTACTCTCTCTTT |
| B2M | 2635 | GCATTCCTGAAGCTGACAGCATTCGGGCCGAGATGTCTCGgcgg |
| | | ccgcCTCCGTGGCCTTAGCTGTGCTCGCGCTACTCTCTCTTTCT |
| B2M | 2636 | ATTCCTGAAGCTGACAGCATTCGGGCCGAGATGTCTCGCTgcgg |
| | | ccgcCCGTGGCCTTAGCTGTGCTCGCGCTACTCTCTCTTTCTGG |
| B2M | 2637 | TCCTGAAGCTGACAGCATTCGGGCCGAGATGTCTCGCTCCgcgg |
| | | ccgcGTGGCCTTAGCTGTGCTCGCGCTACTCTCTCTTTCTGGCC |
| B2M | 2638 | CTGAAGCTGACAGCATTCGGGCCGAGATGTCTCGCTCCGTgcgg |
| | | ccgcGGCCTTAGCTGTGCTCGCGCTACTCTCTCTTTCTGGCCTG |

The electroporated cells were incubated at 37° C. and 500 CO₂ for ˜48 hours to allow for indel formation and knock-in of the donor nucleic acid. DNA was extracted from the electroporated cells 48 hours post-transfection and analyzed by next generation sequencing (NGS). Fluorescence-activated cell sorting (FACS) analysis is performed 5 days post-transfection.

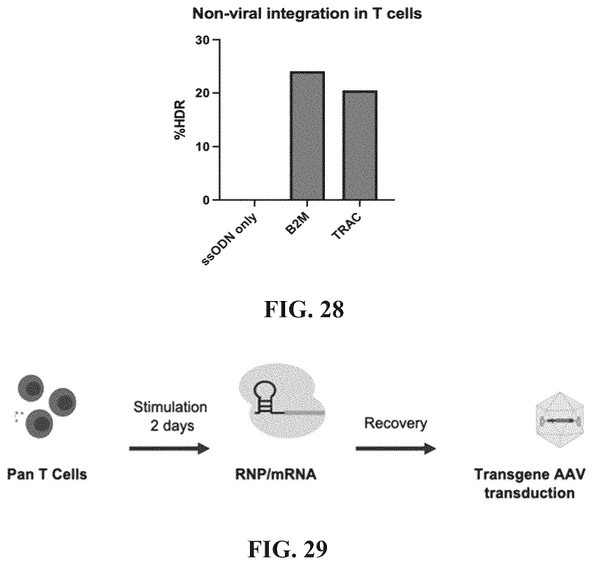

FIG. 28 shows representative data for CasΦ.12 mediated ssODN integration of the donor nucleic acid into the TRAC locus and B32M locus. For negative control, cells were electroporated only with ssODN.

### Example 30. CasΦ.12 L26R Mediated GFP Integration by HDR Pathway in T Cells

The example compares EGFP-CAR integration levels after TRAC knockout with an effector protein by HDR pathway, where the effector protein was delivered by electroporation to T cells either as an RNP complex and an mRNA encoding the effector protein. The effector protein comprised CasΦ.12 L26R having an amino acid sequence of SEQ ID NO: 2592. The guide RNA that was used for the experiment comprised a nucleotide sequence of mC*mU*mU*UCAAGACUAAUAGAUUGCUCCUUACGAGGAGACGAGUCUCUCAGCUG G mU*mA*mC (SEQ ID NO: 2593). FIG. 29 shows schematics of the study design. Briefly, 5×10⁵ activated T cells were electroporated with the guide RNA (500 μmol) and a donor nucleic acid (150 μmol) in combination with either 10 μg of an mRNA encoding the CasΦ.12 L26R (For mRNA transfection) or 250 pmol of CasΦ.12 L26R protein (For RNP complex transfection).

The transfected cells were divided into two portions. The first portion of the transfected cells was incubated at 37° C. and 5% CO₂ to allow for indel formation. The other portion was incubated at 37° C. and 5% CO₂ for 1 hours before AAV transduction. For the AAV transduction, AAV6 particles comprising a donor nucleotide sequence encoding the EGFP-CAR was added at 5×10⁵ MOI of the electroporated T cells for 24 hours. For negative control, untransfected T cells were transduced by the AAV6 particles. The transduced cells were washed of AAV6 particles and further incubated at 37° C. and 5% CO₂ to allow for knock-in of the CD19-CAR. After 6 days post-transfection, the cells were processed by fluorescence-activated cell sorting (FACS) analysis. The results were further confirmed by the next generation sequencing (NGS) analysis.

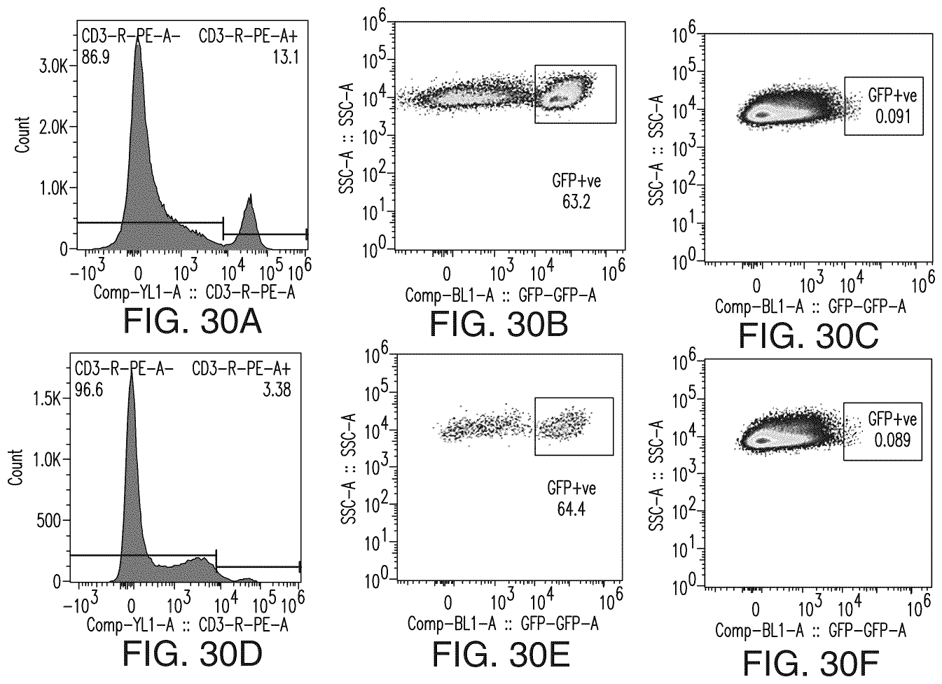

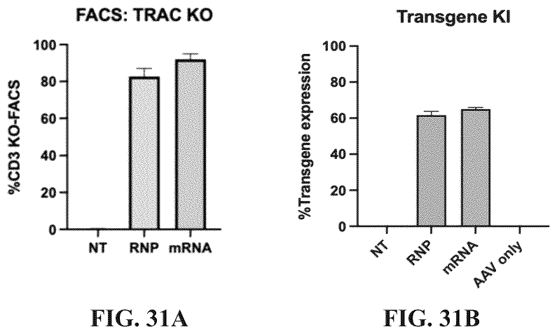

FIGS. 30A and 30D shows that comparable portions of both, the RNP comprising the effector protein and the mRNA encoding the effector protein, treated T cells were not expressing CD3 protein. FIGS. 30B and 30E show that comparable portions of both, the RNP comprising the effector protein and the mRNA encoding the effector protein, treated T cells showed GFP expressing. However, low EGFP-CAR integration was observed on both occasions. Negative controls are shown in FIGS. 30C and 30F, wherein naïve T cells were treated only the AAV6 particles. FIGS. 31A-31B shows alternate representation of the data showed in FIGS. 30A-30F. An analysis of FIG. 31A indicates that both, the RNP transfection and the mRNA transfection, are effective for knocking out TRAC gene in T cells. An analysis of FIG. 31B indicates that both, the RNP transfection and the mRNA transfection, are effective for knocking out TRAC gene, knocking-in EGFP-CAR gene and expressing GFP in T cells. However, it was observed that although GFP integration levels were comparable, the RNP transfection method showed lower editing ability relative to the mRNA transfection method.

### Example 31. Targeted CasΦ.12 L26R Effector Protein Mediated Integration of Promoter-Less CD19-CAR into TRAC Locus

The example demonstrates the generation of T cells with a CD19-specific chimeric antigen receptor (CAR) integrated into the TRAC locus of T cells using an RNP complex and HDR-based insertion method. The T cells that were generated were further tested for their cytotoxic activity on CD19-expressing NALM-6 cells using an LDH release assay.

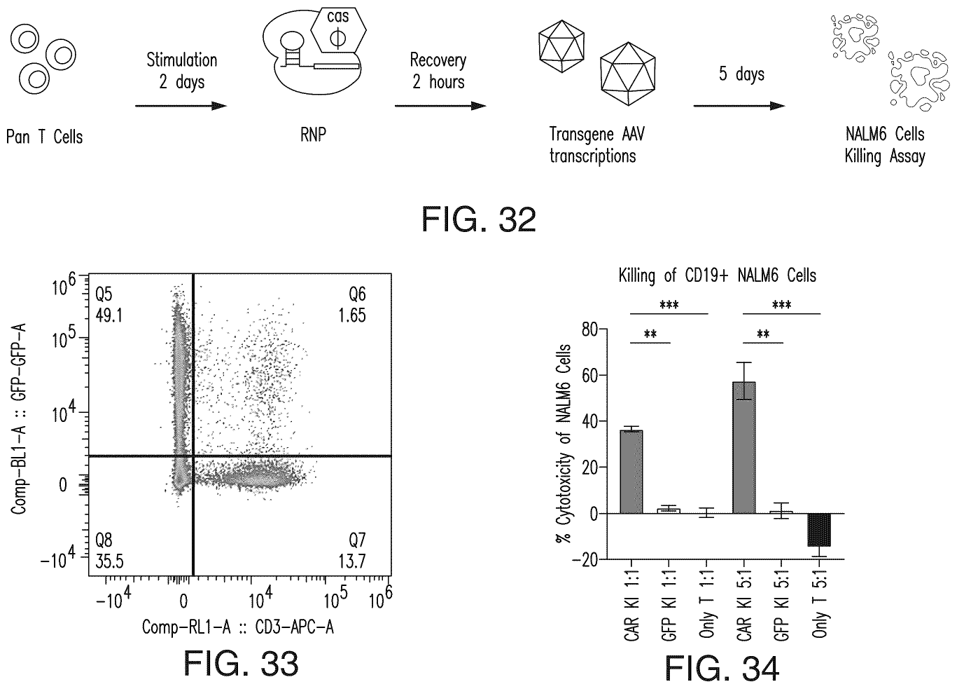

The RNP complex was prepared by incubating 250 pmol of CasΦ.12 L26R effector protein (SEQ ID NO: 2592) and 500 pmol of a guide RNA (mC*mU*mU*UCAAGACUAAUAGAUUGCUCCUUACGAGGAGACGAGUCUCUCAGCUG G mU* mA*mC (SEQ ID NO: 2593)) at room temperature for 30 minutes. FIG. 32 shows schematics of the study design. Briefly, 5×10⁵ activated T cells were electroporated with the RNP complex. The cells were then allowed to recover at 37° C. and 5% CO₂ for 2 hours before AAV transduction. For the AAV transduction, AAV6 particles containing a donor nucleotide sequence encoding the CD19-CAR or a donor nucleotide sequence encoding GFP was added at 1×10⁵ MOI of the transfected T cells. The transduced cells were allowed to recover at 37° C. and 5% CO₂ for 5 days. The transduced cells were then processed for fluorescence-activated cell sorting (FACS) analysis to determine knock-in of the donor nucleotide sequence. The cells transduced with the donor nucleotide encoding CD19-CAR, no signal was observed for the CD19-CAR construct on the cell surface. Similarly, as shown in FIG. 33, the cells transduced with the donor nucleotide encoding GFP, about 49% of TRAC knock out cells were observed to have GFP integration. The results were further confirmed by the next generation sequencing (NGS) analysis.

For the NALM6 cell killing assay, the transduced cells were further processed through magnetic bead separation method for enriching CD3⁻ cells from about 87.7% CD3⁻ cells before sorting to 97.2% CD3⁻ cells after sorting. The CD3⁻ cells were then incubated with NALM6 cells in a supporting media at a ratio of 50000:10000 and 10000:10000 for 24 hours at 37° C. After 24 hours, specific cytotoxicity of the NALM6 cells by CD19-CAR knock-in cells, GFP knock-in cells and control untreated T cells was quantified by a colorimetric assay by determining an amount of lactate dehydrogenase (LDH) released from the cells. % cytotoxicity was calculated using formula 1.

$$\% Cytotoxicity = [ \frac{\begin{matrix}( Experimental - T cell Spont . Release - \\ NLM 6 Spont . Release )\end{matrix}}{( NALM 6 Max . Release - T cell Spont . Release )} ] \times 100 \qquad Formula 1$$

Specific cytotoxicity of the NALM6 cells by CD19-CAR knock-in cells, GFP knock-in cells and control untreated T cells is shown in FIG. 34. An analysis of FIG. 34 indicates that CD19-CAR knock-in cells showed significantly higher cell killing than GFP knock-in cells.

### Example 32. Evaluation of T Cell Fitness Post Gene Editing by CasΦ.12 L26R

The example demonstrates B2M knock out ability of CasΦ.12 L26R effector proteins in T cells and T cell memory profiles that had B2M gene knocked out.

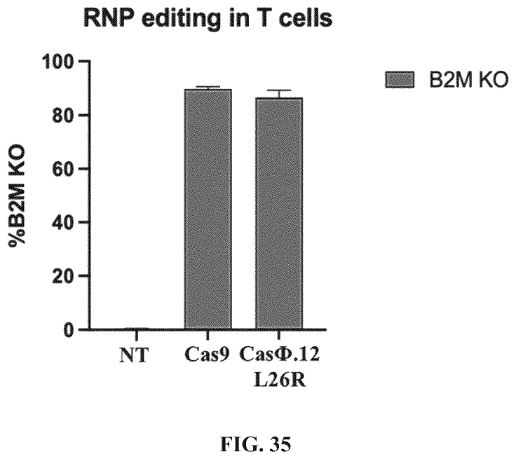

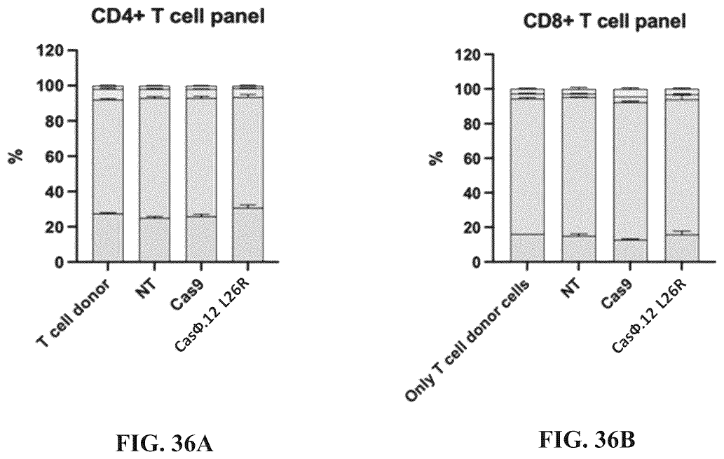

The RNP complex was prepared by incubating 250 pmol of CasΦ.12 L26R effector protein (SEQ ID NO: 2592) and 500 pmol of a guide RNA (mC*mU*mU*UCAAGACUAAUAGAUUGCUCCUUACGAGGAGACAGCAAGGACUGGUC mU*mU*mU (SEQ ID NO: 2640)) at room temperature for 30 minutes. Briefly, 5×10⁵ activated T cells were electroporated with the RNP complex. The cells were then allowed to recover at 37° C. and 5% CO₂ for 72 hours. The transduced cells were then processed for fluorescence-activated cell sorting (FACS) analysis to determine knock out in B2M locus as well as T cell memory profile. Cas9 system was used as a positive control. As shown in FIG. 35, CasΦ.12 L26R effector protein showed high level of editing. Two experiments were conducted for determining T cell memory profiles: (1) CD4+ T cell panel (FIG. 36A); and (2) CD8+ T cell panel (FIG. 36B).

An analysis of FIGS. 36A-36B indicates that T cells were able to maintain fitness after CasΦ.12 L26R effector protein mediated gene editing treatment.

### Example 33. Evaluation of T Cell Fitness Post Gene Editing by CasΦ.12 and Variants Thereof at Low Dose

The example demonstrates T cell memory profiles that had B2M gene knocked out by CasΦ.12 effector protein (SEQ ID NO: 57), CasΦ.12 L26R effector protein (SEQ ID NO: 2592), or CasM.265466 effector protein (SEQ ID NO: 2435). Cas9 effector protein was used as a positive control.

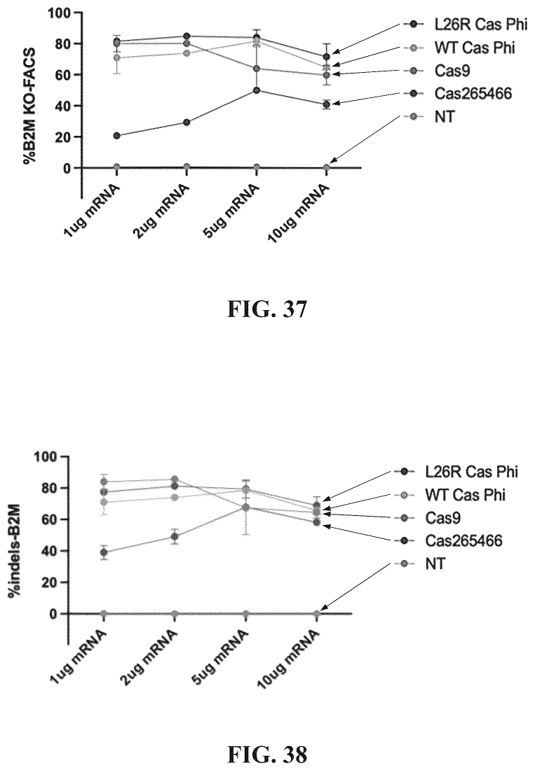

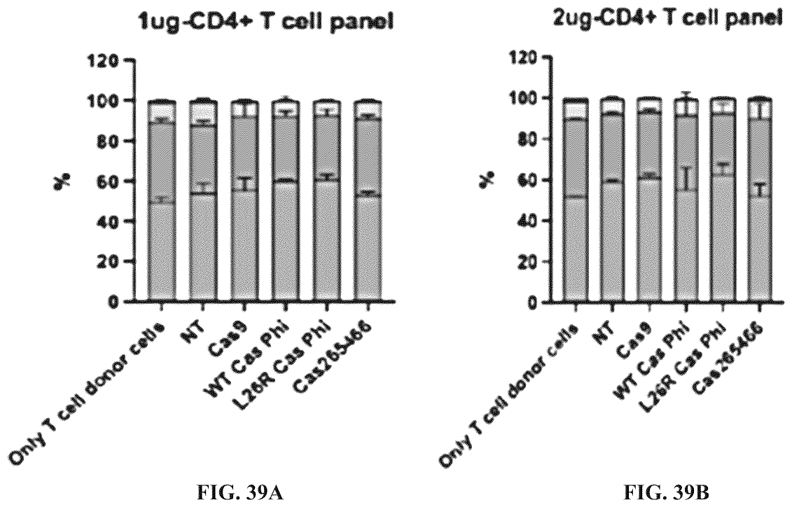

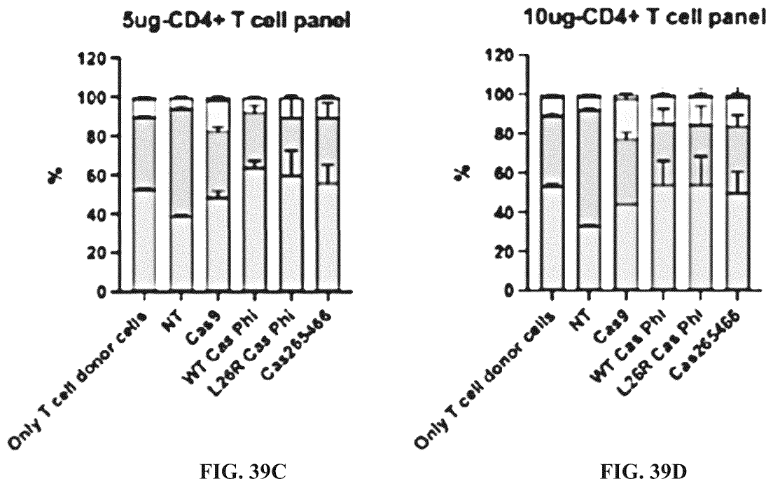

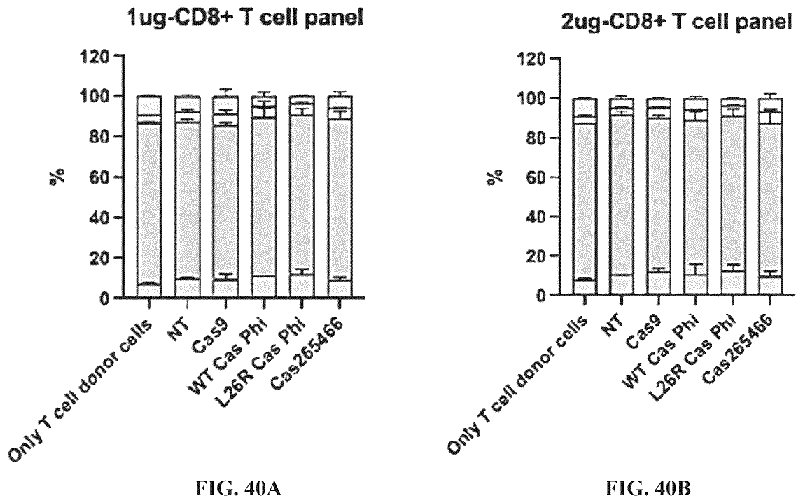

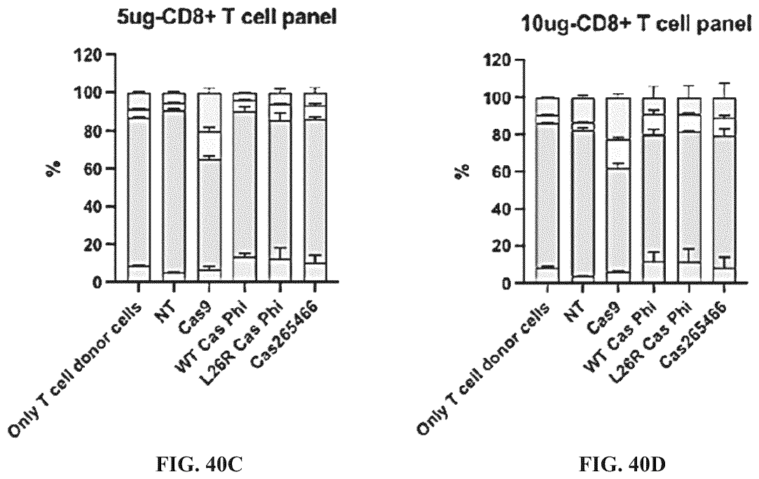

Briefly, 3×10⁵ activated T cells were electroporated with 500 μM of a guide RNA and an mRNA encoding the effector protein at 1 μg, 2 μg, 5 μg or 10 μg concentration. With CasΦ.12 and CasΦ.12 L26R, the guide RNA of SEQ ID NO: 2640 was used. With CasM.265466, the guide RNA of SEQ ID NO: 2448 was used. The cells were then allowed to recover at 37° C. and 5% CO₂. The transduced cells were then processed for fluorescence-activated cell sorting (FACS) analysis to determine knock out in B2M locus (FIG. 37). The knock-out results of FACS were further confirmed by NGS analysis (FIG. 38). Additionally, two experiments were conducted for determining T cell memory profiles: (1) CD4+ T cell panel (FIGS. 39A-39D); and (2) CD8+ T cell panel (FIGS. 40A-40D).

An analysis of FIGS. 39A-39D and 40A-40D indicates that T cell maintained fitness after CasΦ.12 effector protein, CasΦ.12 L26R effector protein or CasM.265466 effector protein mediated gene editing treatment.

### Example 34. Off-Target Sites in Primary T Cells for Guide RNA Targeting B2M Gene and CasΦ.12

The example demonstrates a guide RNA that was targeting the B2M gene were found to have high specificity in primary T cells. CasΦ.12 effector protein comprising an amino acid sequence of SEQ ID NO: 57 was used. The guide RNA comprises a nucleotide sequence of SEQ ID NO: 1381. T cells were electroporated with 500 pmol of guide RNA and 20 μg of CasΦ.12 effector mRNA. 29 off-target sites in primary T cells were tested for the guide RNA.

Only three off-target sites with detectable indels (>0.1% indel) were observed. Extrapolating the results, % of reads modified at off-target sites were calculated to be 1.92%, 1.27% and 0.42%, respectively.

### Example 35. Off-Target Sites in Primary T Cells for Guide RNA Targeting TRAC Gene and CasΦ.12

The example demonstrates a guide RNA that was targeting the TRAC gene was found to have high specificity in primary T cells. CasΦ.12 effector protein comprising an amino acid sequence of SEQ ID NO: 57 was used. The guide RNA comprises a nucleotide sequence of SEQ ID NO: 1382. T cells were electroporated with 500 pmol of guide RNA and 20 μg of CasΦ.12 effector mRNA. 25 off-target sites in primary T cells were tested for the guide RNA.

Only two off-target sites with detectable indels (>0.1% indel) were observed. Extrapolating the results, % of reads modified at off-target sites were calculated to be 0.26% and 0.25%, respectively.

### Example 36: PAM Screening for CasM.265466 Effector Protein

CasM.265466 effector protein and guide RNA combinations represented in TABLE 38 were screened by in vitro enrichment (IVE) for PAM recognition. The CasM.265466 comprises amino acid sequence of SEQ ID NO: 2435. The nucleotide sequences of the guide components are shown in TABLE 38. For example, as shown in TABLE 38, the effector protein complexed with a guide comprising a crRNA of SEQ ID NO: 2594 and a tracrRNA of SEQ ID NO: 2597 was screened for PAM recognition.

TABLE 38
Compositions for PAM Sequence Recognition

| | Effector | | |
|---|---|---|---|
| | protein | | |
| Composition | (SEQ ID | guide RNA | tracrRNA |
| No. | NO:) | (SEQ ID NO:) | (SEQ ID NO:) |
| 1 | 2435 | GUUUGAGAACCUUAUGAA | ACAGCUUAUUUGGAAGCU |
| | | AUUACAAGGAUGCCAAAC | GAAAUGUGAGGUUUAUAA |
| | | UAUUAAAUACUCGUAUUG | CACUCACAAGAAUCCU |
| | | CU (SEQ ID NO: | (SEQ ID NO: 2597) |
| | | 2594) | |
| 2 | 2435 | GUUUGAGAACCUUAUGAA | UAUAUUUGAUAAAAAUAU |
| | | AUUACAAGGAUGCCAAAC | ACAGCUUAUUUGGAAGCU |
| | | UAUUAAAUACUCGUAUUG | GAAAUGUGAGGUUUAUAA |
| | | CU (SEQ ID NO: | CACUCACAAGAAUCC |
| | | 2595) | (SEQ ID NO: 2598) |
| 3 | 2435 | ACAGCUUAUUUGGAAGCU | |
| | | GAAAUGUGAGGUUUAUAA | |
| | | CACUCACAAGAAUCCUGA | |
| | | AAAAGGAUGCCAAACUAU | |
| | | UAAAUACUCGUAUUGCU | |
| | | (SEQ ID NO: 2596) | |

Briefly, effector proteins were complexed with corresponding guide RNAs for 15 minutes at 37° C. The complexes were added to an IVE reaction mix. PAM screening reactions used 10 μl of RNP in 100 μl reactions with 1,000 ng of a 5′ PAM library in 1× Cutsmart buffer and were carried out for 15 minutes at 25° C., 45 minutes at 37° C. and 15 minutes at 45° C. Reactions were terminated with 1 μl of proteinase K and 5 μl of 500 mM EDTA for 30 minutes at 37° C. Next generation sequencing was performed on cut sequences to identify enriched PAM sequence for CasM.265466 as shown in TABLE 39. Cis cleavage by each complex was confirmed by gel electrophoresis.

The most enriched PAM was represented by the sequence 5′-TNTR-3′, wherein N is any nucleotide and R is adenine or guanine.

The assay conducted in this example can also be repeated using CasM.292007 (SEQ ID NO: 2599). Based on significant homology between SEQ ID NO: 2435 and SEQ ID NO: 2599, and based on the results described above, the PAM for CasM.292007 is predicted to be 5′-TNTR-

TABLE 39
Exemplary PAM Sequences
PAM Sequence

NNTNTR
TNTR

wherein each N is independently any one of A, G, C, or T.
wherein each R is independently any one of A, or G.

### Example 37: Additional PAM Screening for CasM.265466

Prior in vitro screening as described in Example 38 for CasM.265466 effector protein (SEQ ID NO: 2435) PAM recognition demonstrated that the most enriched PAM sequence for CasM.265466 was a TNTR PAM sequence, but also indicated that the effector protein may tolerate a more flexible PAM sequences beyond TNTR without significantly compromising nuclease activity. Effector protein and flexible PAM group combinations as set forth in TABLE 40 were screened to confirm that chromosomal DNA may be efficiently targeted in mammalian cells (HEK293T) using a more flexible PAM sequence.

Single and double point mutations were made along TNTR.

TABLE 40
PAM SEQUENCES
PAM Group*

NNTN
ANTR
CNTR
GNTR
TNAR
TNCR
TNGR
TNTC
TNTT
VNTY
TNVY

*wherein each N is any nucleotide, each R is A or G, and each V is A, C or G.

At least six spacers that previously showed >3% indel rate were selected for each PAM group identified in TABLE 40.

Single guide nucleic acids (sgRNA) comprising the handle sequence of SEQ ID NO: 2522 linked to a 20 nt spacer sequence.

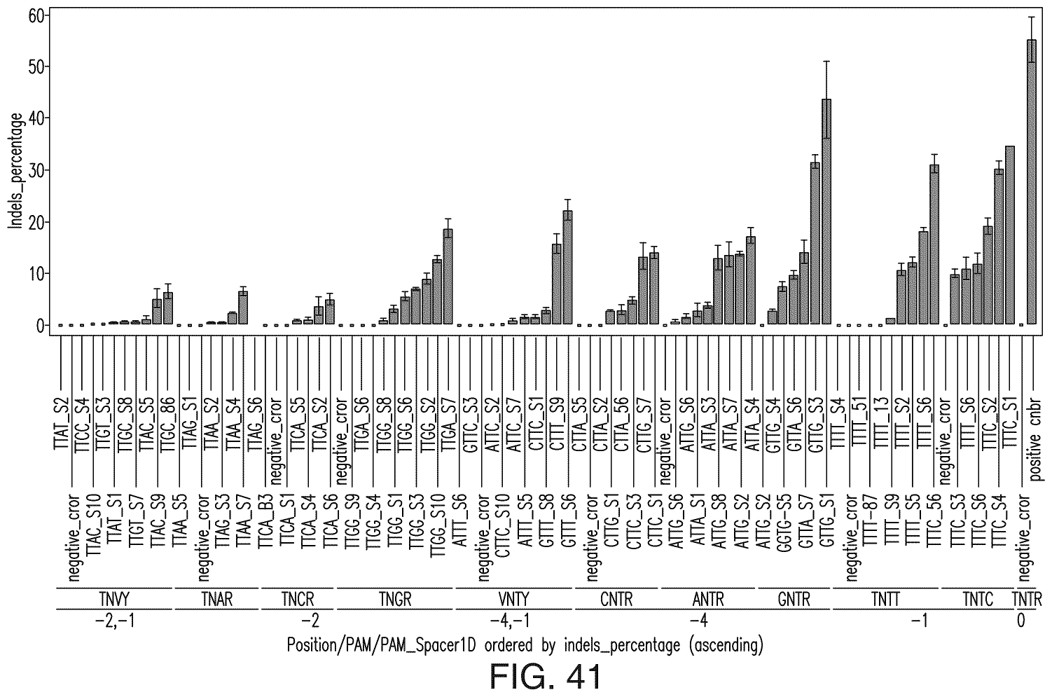

Plasmids encoding CasM.265466 effector protein and plasmids encoding the sgRNAs were delivered by lipofection to HEK293T cells and permitted to grow to allow for indel formation. Cells were lysed and indels were detected by next generation sequencing. Indel percentage was calculated and plotted as shown in FIG. 41.

While the top performing complexes were found to produce up to or greater than 30% indel, the data also demonstrates that single and double point mutations at ˜4 and −1 were the most permissive for allowing nuclease activity. Furthermore, the CasM.265466 effector protein complexed with two different sgRNAs having different spacer sequences generated 20% indel at targeted sequences adjacent to an NNTN PAM. Therefore, these results further confirm the results of Example 36 and demonstrate that the CasM.265466 effector protein recognizes a flexible NNTN PAM sequence.

### Example 38. CasM.265466 Mediated GFP Integration in T Cells

This example demonstrates the generation of T cells having a GFP marker integrated into the TRAC locus of T cells using RNP complexes of CasM.265466 having an amino acid sequence of SEQ ID NO: 2435, and a TRAC specific guide RNA having a sequence of SEQ ID NO: 2488, 2489 or 2490. Briefly, 2.5×10⁶ activated T cells were electroporated with a mixture of an mRNA encoding the CasM.265466 (10 μg) and an mRNA encoding the TRAC specific guide RNA (500 μmol). The transfected cells were divided into two portions. The first portion of the transfected cells were incubated at 37° C. and 5% CO₂ for ˜72 hours to allow for indel formation. The other portion was incubated at 37° C. and 5% CO₂ for 2 hours before AAV transduction. For the AAV transduction, AAV6 particles containing a donor nucleotide sequence encoding the GFP marker was added at 5×10⁵ MOI of the electroporated T cells for 24 hours. The transduced cells were washed of AAV6 particles and further incubated at 37° C. and 5% CO₂ for 48 hours to allow for knock in of the GFP marker. After 6 days post AAV addition, the cells were processed by fluorescence-activated cell sorting (FACS) analysis and next generation sequencing (NGS) analysis. Indel percentage was calculated as the fraction of sequencing reads containing insertions or deletions relative to an unedited reference sequence. For negative control, AAV6 particles containing a donor nucleotide sequence encoding the GFP marker was used with activated naïve T cells.

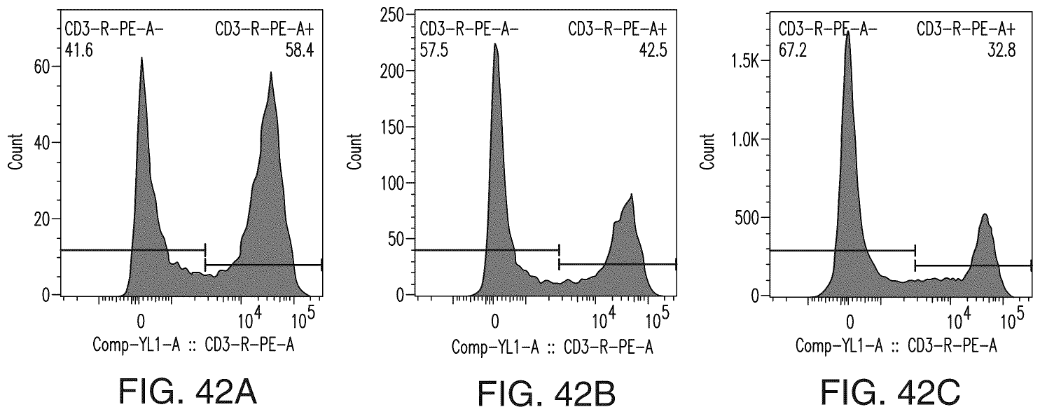

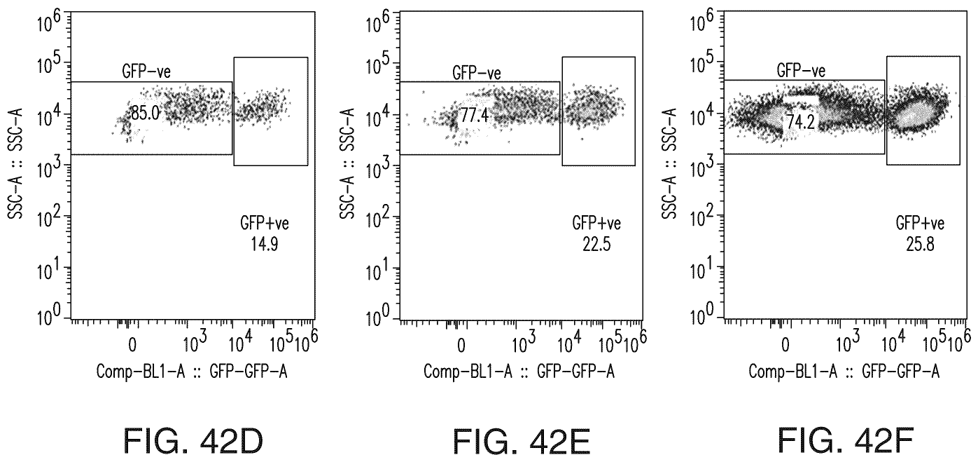

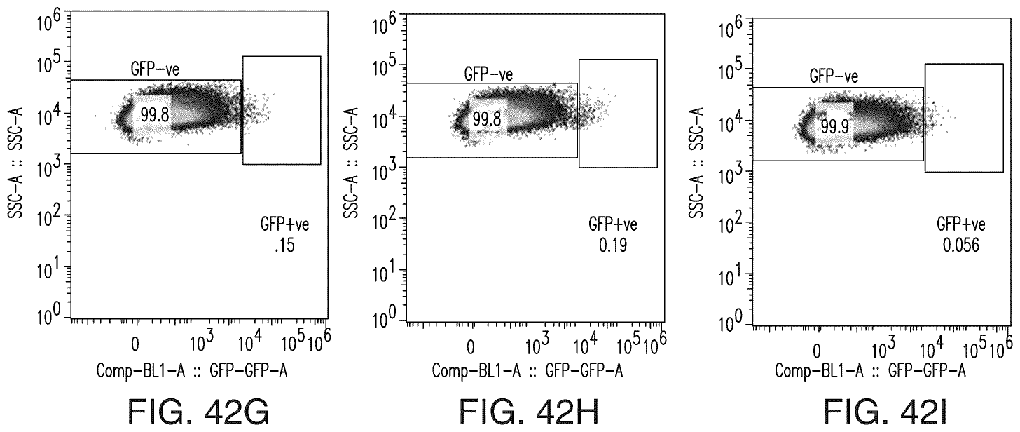

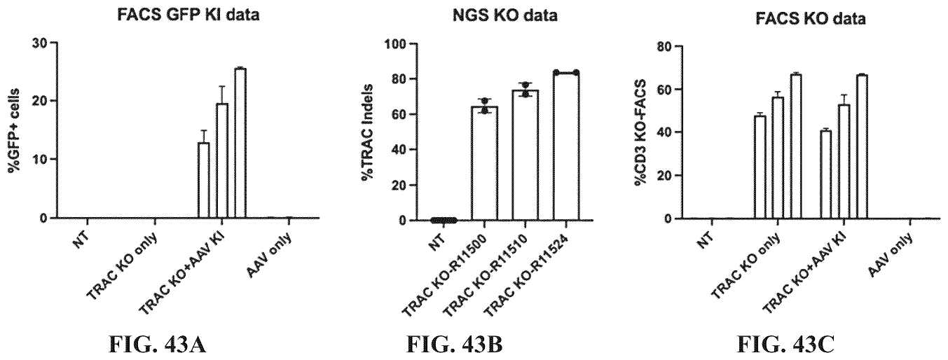

An analysis of FIGS. 42A-42C and 43A indicates that all three guide RNAs were able to successfully knock out TRAC gene. An analysis of FIGS. 42D-42F and 43C indicates that GFP was successfully integrated into TRAC locus after treatment with the RNP complex. FIGS. 42G-42I show results of the negative control that did not show GFP expression. The results were further confirmed by NGS analysis (FIG. 43B). In conclusion, the study shows that a donor nucleic acid can be integrated into the TRAC locus using the method described herein.

### Example 39. Targeted CasM.265466 Effector Protein Mediated Integration of Promoter-Less CD19-CAR into TRAC Locus

The example demonstrates the generation of T cells with a CD19-specific chimeric antigen receptor (CAR) integrated into the TRAC locus of T cells using an RNP complex and HDR-based insertion method. The T cells that are generated are further tested for their cytotoxic activity on CD19-expressing NALM-6 cells using an LDH release assay.

The RNP complex is prepared by incubating 250 pmol of CasM.265466 effector protein (SEQ ID NO: 2435) and 500 pmol of a guide RNA (SEQ ID NO: 2490) at room temperature for 30 minutes. FIG. 32 shows schematics of the study design. Briefly, 5×10⁵ activated T cells are electroporated with the RNP complex. The cells are then allowed to recover at 37° C. and 5% CO₂ for 2 hours before AAV transduction. For the AAV transduction, AAV6 particles containing a donor nucleotide sequence encoding the CD19-CAR or a donor nucleotide sequence encoding GFP are added at 1×10⁵ MOI of the transfected T cells. The transduced cells are allowed to recover at 37° C. and 5% CO₂ for 5 days. The transduced cells are then processed for fluorescence-activated cell sorting (FACS) analysis to determine knock-in of the donor nucleotide sequence. The results are further confirmed by the next generation sequencing (NGS) analysis.

For the NALM6 cell killing assay, the transduced cells are further processed through magnetic bead separation method for enriching CD3⁻ cells. The CD3⁻ cells are then incubated with NALM6 cells in a supporting media at a ratio of 50000:10000 and 10000:10000 for 24 hours at 37° C. After 24 hours, specific cytotoxicity of the NALM6 cells by CD19-CAR knock-in cells, GFP knock-in cells and control untreated T cells is quantified by a colorimetric assay by determining an amount of lactate dehydrogenase (LDH) released from the cells. % cytotoxicity was calculated using formula 1.

### Example 40. Off-Target Sites in Primary T Cells for CasM.265466 Effector Protein and Guide RNA Targeting B2M Gene

The example demonstrates three guide RNAs that were targeting the B2M gene were found to have high specificity in primary T cells. CasM.265466 effector protein comprising an amino acid sequence of SEQ ID NO: 2435 was used. Three guide RNAs, each having a handle sequence of SEQ ID NO: 2523 and a spacer sequence of SEQ ID NO: 2439, 2448, or 2450, were tested. T cells were electroporated with guide RNA and Cas 265466 effector mRNA at the following concentration ratios: 1) 5 μg Cas 265466 mRNA and 500 pmol guide RNA; 2) 10 μg Cas 265466 mRNA and 500 pmol guide RNA; 3) 10 μg Cas 265466 mRNA and 1000 pmol guide RNA; 4) 20 μg Cas 265466 mRNA and 500 pmol guide RNA; and 5) 20 μg Cas 265466 mRNA and 1000 pmol guide RNA. 18, 17 and 11 off-target sites in primary T cells were tested for the guides having spacer sequences of SEQ ID NO: 2439, 2448, and 2450, respectively.

Only one off-target site with detectable indels (>0.1% indel) was observed for the guides having spacer sequences of SEQ ID NO: 2439 and 2450, respectively. Extrapolating the results, % of reads modified at off-target sites were calculated to be 0.47% and 0.56%, respectively.

### Example 41. Off-Target Sites in Primary T Cells for CasM.265466 Effector Protein and Guide RNA Targeting TRAC Gene

The example demonstrates three guide RNAs that were targeting the TRAC gene were found to have high specificity in primary T cells. CasM.265466 effector protein comprising an amino acid sequence of SEQ ID NO: 2435 was used. Three guide RNAs, each having a handle sequence of SEQ ID NO: 2523 and a spacer sequence of SEQ ID NO: 2452, 2462 or 2476, were tested. T cells were electroporated with guide RNA and Cas 265466 effector mRNA at the following concentration ratios: 1) 5 μg Cas 265466 mRNA and 500 pmol guide RNA; 2) 5 μg Cas 265466 mRNA and 1000 pmol guide RNA; 3) 10 μg Cas 265466 mRNA and 500 pmol guide RNA; 4) 10 μg Cas 265466 mRNA and 500 pmol guide RNA; and 5) 10 μg Cas 265466 mRNA and 1000 pmol guide RNA. 9, 7 and 5 off-target sites in primary T cells were tested for the guides having spacer sequences of SEQ ID NO: 2452, 2462 and 2476, respectively.

No off-target sites with detectable indels (>0.1% indel) was observed for any of the three guide RNAs tested.

### Example 42. Off-Target Sites in Primary T Cells for CasM.265466 Effector Protein and Guide RNA Targeting CIITA Gene

The example demonstrates three guide RNAs that were targeting the CIITA gene were found to have high specificity in primary T cells. CasM.265466 effector protein comprising an amino acid sequence of SEQ ID NO: 2435 was used. Three guide RNAs, each having a handle sequence of SEQ ID NO: 2523 and a spacer sequence of SEQ ID NO: 2488, 2489 or 2490, were tested. T cells were electroporated with guide RNA and Cas 265466 effector mRNA at the following concentration ratios: 1) 5 μg Cas 265466 mRNA and 500 pmol guide RNA; 2) 5 μg Cas 265466 mRNA and 1000 pmol guide RNA; 3) 10 μg Cas 265466 mRNA and 500 pmol guide RNA; and 4) 10 μg Cas 265466 mRNA and 1000 pmol guide RNA. 30, 15 and 8 off-target sites in primary T cells were tested for the guides having spacer sequences of SEQ ID NO: 2488, 2489 and 2490, respectively.

Only two off-target sites with detectable indels (>0.1% indel) were observed for the guide having a spacer sequence of SEQ ID NO: 2490. Extrapolating the results, % of reads modified at off-target sites were calculated to be 0.8% and 1.8%, respectively.

### Example 43. Arginine Mutation Scanning of CasM.265466 to Identify Charge Substitution Rules of Effector Protein Activity

CasM.265466 arginine mutants were tested for their ability to produce indels in HEK293T cells. A total of 368 arginine mutants were tested. Briefly, a first plasmid encoding a CasM.265466 arginine mutant and a second plasmid encoding a single guide RNA were delivered by lipofection to HEK293T cells. The sgRNA comprised a nucleotide sequence of ACAGCUUAUUUGGAAGCUGAAAUGUGAGGUUUAUAACACUCACAAGAAUCCUGAAAAA GGAUGCCAAACUCUUCGCCCAGAGCAUCCCA (SEQ ID NO: 2600). The sgRNA comprised a spacer sequence that was designed to hybridize to a target sequence adjacent to a PAM of TNTR (e.g., TTTG). For lipofections, 15 ng of the nuclease mutant and 150 ng of the guide RNA encoding plasmid were delivered to ˜30,000 HEK293T cells in 200 μl using TransIT-293 lipofection reagent. Lipofected cells were grown for ˜72 hrs at 37° C. to allow for indel formation. Indels were detected by next generation sequencing of PCR amplicons at the targeted loci and indel percentage was calculated as the fraction of sequencing reads containing insertions or deletions relative to an unedited reference sequence. Sequencing libraries with less than 20% of reads aligning to the reference sequence were excluded from the analysis for quality control purposes. Wildtype CasM.265466 was included as positive control and reference for the mutants.

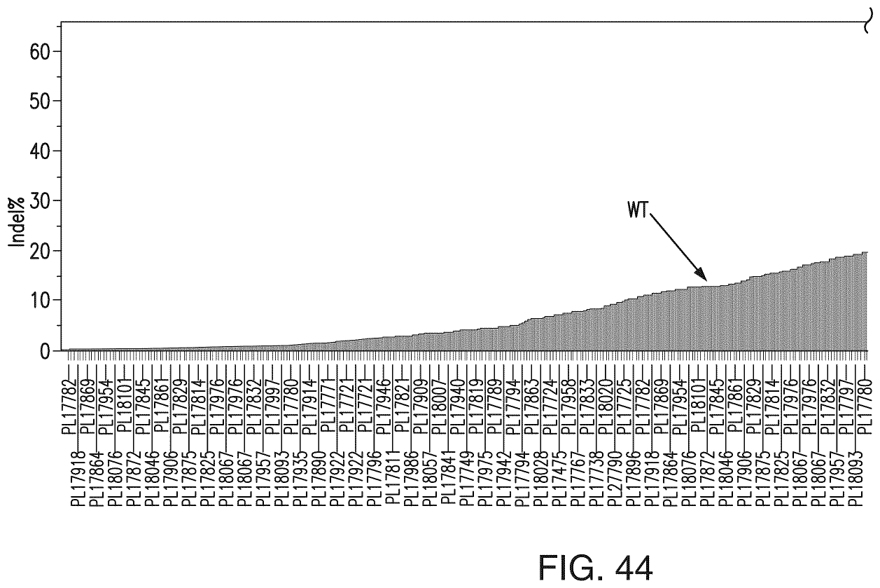

The mean indel percentage for each of the arginine mutant is shown in FIG. 44. An analysis of FIG. 44 indicates that positive charge of arginine may strengthen the interaction between the effector protein and the negatively charged DNA backbone. Top 10 arginine mutants that showed increase in indel potency includes I80R, T84R, K105R, G210R, C202R, A218R, D220R, E225R, C246R, and Q360R.

### Example 44. CasM.265466 Arginine Mutants and their Potency for Indel Generation

The top ten nuclease mutants, each comprising different CasM.265466 arginine mutant, as identified in Example 43 were tested for their ability to produce indels in HEK293T cells over a variety of doses. Briefly, a first plasmid encoding a CasM.265466 mutant and a second plasmid encoding a single guide RNA (sgRNA) were delivered by lipofection to HEK293T cells. The sequence of the sgRNAs included a nucleotide sequence of

(SEQ ID NO: 2600)
ACAGCUUAUUUGGAAGCUGAAAUGUGAGGUUUAUAACACUCACAAGAAUC
CUGAAAAAGGAUGCCAAACUCUUCGCCCAGAGCAUCCCA.

The sgRNA spacer was designed to hybridize to a target sequence adjacent to a PAM of TNTR (e.g., TTTG). For lipofections, the CasM.265466 mutant and sgRNA were delivered to ˜30,000 HEK293T cells in 200 μl using TransIT-293 lipofection reagent. Each of the ten nuclease mutants were tested at a dose ranging from 1.17 ng to 150 ng. The sgRNA encoding plasmid was used at a concentration of 150 ng. Lipofected cells were grown for ˜72 hrs at 37° C. to allow for indel formation. Indels were detected by next generation sequencing of PCR amplicons at the targeted loci and indel percentage was calculated as the fraction of sequencing reads containing insertions or deletions relative to an unedited reference sequence. Sequencing libraries with less than 20% of reads aligning to the reference sequence were excluded from the analysis for quality control purposes. Wildtype CasM.265466 was included as positive control and reference for the mutants.

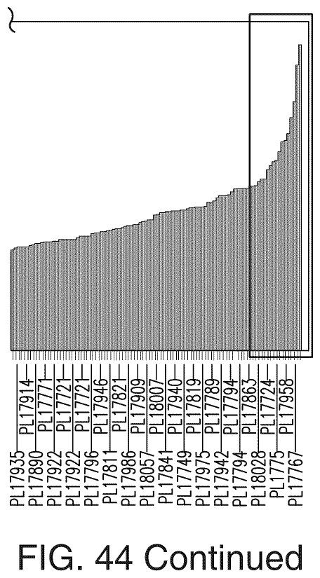

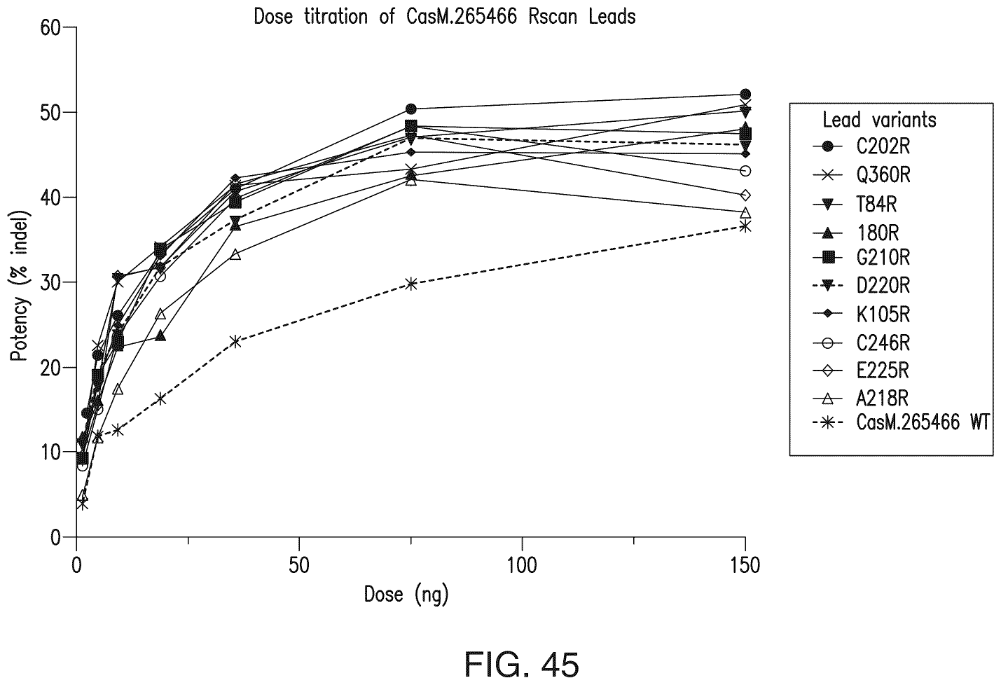

The mean indel percentage and standard deviation based on three replicates is reported in FIG. 45. An analysis of FIG. 45 indicates that arginine substitution can increase potency of the effector protein in the generation of indels.

### Example 45. CasM.265466 and D220R Variant Thereof for MLH1 Gene Editing in HEK293T Cells

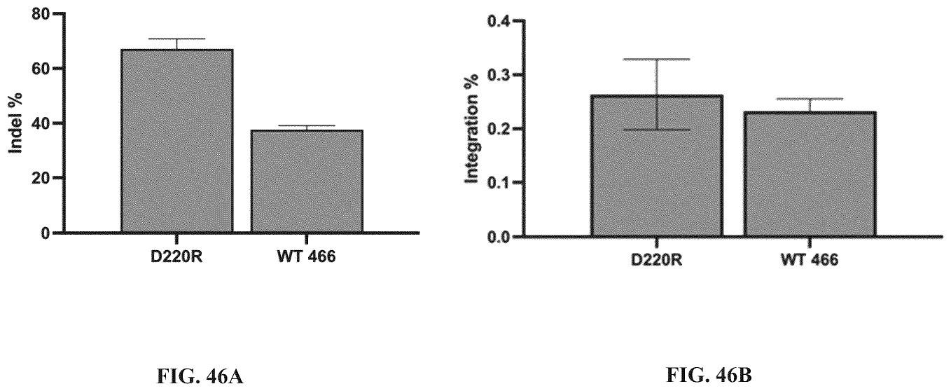

The purpose of this study was to test guide nucleic acids for MLH1 gene knockout with CasM.265466 effector protein and D220R variant thereof by electroporation in HEK293T cells. The CasM.265466 effector protein comprised an amino acid sequence of SEQ ID NO: 2435. The D220R variant comprised an amino acid sequence of SEQ ID NO: 2601. The guide RNA comprised a handle sequence of SEQ ID NO: 2522 linked to a spacer sequence of AGUCUCCAGGAAGAAAUUAA (SEQ ID NO: 2602). Briefly, 2.3×10⁵ HEK293T cells were electroporated with 0.75 μg of the effector protein mRNA, 1.25 μg of guide RNA, and 100 pmol of a donor nucleic acid. The cells were then allowed to recover at 37° C. and 5% CO₂. DNA was extracted 72 hours post-transfection and % indel generation and donor nucleic acid insertion was measured by NGS analysis (FIGS. 46A-46B).

As shown in FIG. 46A, the D220R variant showed % indel twice relative to the corresponding wildtype CasM.265466 effector protein. However, in contrast, the D220R variant did not improve insertion of the donor nucleic acid relative to the corresponding wildtype CasM.265466 effector protein (FIG. 46B).

### Example 46. CasM.265466 D220R Variant B2M Gene Editing Studies in T Cells

The purpose of this study was to test CasM.265466 D220R variant effector protein (SEQ ID NO: 2601) for B2M knockout relative to corresponding wildtype CasM.265466 effector protein (SEQ ID NO: 2435) in T cells. The guide RNA comprises a handle sequence of SEQ ID NO: 2522 linked to a spacer sequence of SEQ ID NO: 1637. Briefly, 3×10⁵ activated T cells were electroporated with the guide RNA at a concentration of 500 pmol and the effector protein mRNA at a concentration of 0.5 μg, 1 μg, 2 μg, 5 μg, or 10 μg. After 72 hours post-transfection, the cells were processed by fluorescence-activated cell sorting (FACS) analysis and next generation sequencing (NGS) analysis.

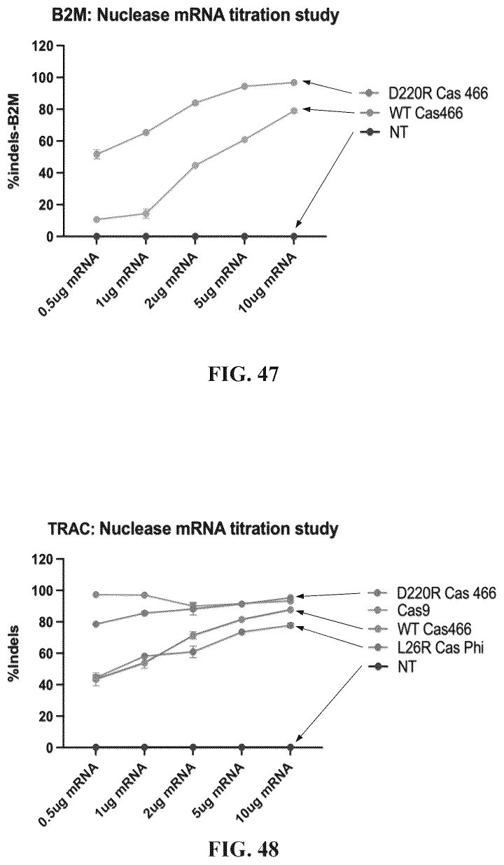

An analysis of the NGS results in FIG. 47 indicates that the CasM.265466 D220R variant effector protein showed improved gene editing in primary human T cells relative to corresponding wildtype CasM.265466 effector protein.

### Example 47. CasM.265466 D220R Variant TRAC Gene Editing Studies in T Cells

The purpose of this study was to test CasM.265466 D220R variant effector protein (SEQ ID NO: 2601) for TRAC knockout relative to corresponding wildtype CasM.265466 effector protein (SEQ ID NO: 2435), and CasΦ.12 L26R variant effector protein (SEQ ID NO: 2592) in T cells. Cas9 effector protein was used as a positive control. The guide RNA for CasM.265466 D220R variant effector protein and corresponding CasM.265466 effector protein comprised a handle sequence of SEQ ID NO: 2522 linked to a spacer sequence of SEQ ID NO: 1986. The guide RNA for CasΦ.12 L26R variant effector protein comprised a guide RNA sequence of mC*mU*mU*UCAAGACUAAUAGAUUGCUCCUUACGAGGAGACGAGUCUCUCAGCUG GmU*mA*mC (SEQ ID NO: 2641). Briefly, 3×10⁵ activated T cells were electroporated with the guide RNA at a concentration of 500 pmol and the effector protein mRNA at a concentration of 0.5 μg, 1 μg, 2 μg, 5 μg, or 10 μg. After 72 hours post-transfection, the cells were processed by fluorescence-activated cell sorting (FACS) analysis and next generation sequencing (NGS) analysis.

An analysis of the NGS results in FIG. 48 indicates that the CasM.265466 D220R variant effector protein showed improved editing in primary human T cells relative to corresponding wildtype CasM.265466 effector protein and CasΦ.12 L26R variant effector protein.

1-271. (canceled)

272. An engineered T cell comprising a gene that is modified by contacting a T cell with:
(a) an effector protein comprising an amino acid sequence that is at least 95% identical to SEQ ID NO: 2435; and
(b) a guide nucleic acid, wherein the guide nucleic acid comprises a nucleotide sequence that is at least 90% identical to 5′-AAGGAUGCCAAAC-3′ (nucleotides 57 to 69 of SEQ ID NO: 2522) and a spacer sequence that is complementary to a target sequence of the gene.

273. The engineered T cell of claim 272, wherein the effector protein comprises an amino acid sequence that is at least 98% identical to SEQ ID NO: 2435.

274. The engineered T cell of claim 272, wherein the T cell is a primary human T cell.

275. The engineered T cell of claim 272, wherein the engineered T cell comprises a nucleic acid encoding a chimeric antigen receptor (CAR).

276. The engineered T cell of claim 272, wherein the effector protein and guide nucleic acid recognize a protospacer adjacent motif (PAM) selected from 5′-NNTN-3′ and 5′-TNTR-3′.

277. The engineered T cell of claim 272, wherein the effector protein is fused to a fusion partner protein.

278. The engineered T cell of claim 277, wherein the fusion partner protein comprises polymerase activity.

279. The engineered T cell of claim 272, wherein the effector protein comprises an amino acid substitution relative to SEQ ID NO: 2435 selected from I80R, T84R, K105R, G210R, C202R, A218R, E225R, C246R, and Q360R.

280. The engineered T cell of claim 272, wherein the engineered T cell further comprises a single-stranded oligodeoxynucleotide that is integrated into the gene.

281. The engineered T cell of claim 272, wherein the gene is modified by an additional guide nucleic acid that comprises the nucleotide sequence that is at least 90% identical to 5′-AAGGAUGCCAAAC-3′ (nucleotides 57 to 69 of SEQ ID NO: 2522), and an additional spacer sequence that is complementary to a different target sequence of the gene.

282. The engineered T cell of claim 272, wherein the effector protein comprises nuclease activity.

283. The engineered T cell of claim 272, wherein the effector protein comprises nickase activity.

284. The engineered T cell of claim 272, wherein at least one phosphodiester bond of the gene is cleaved relative to its unmodified state.

285. The engineered T cell of claim 272, wherein at least one nucleotide is deleted from the gene, at least one nucleotide is inserted into the gene, at least one nucleotide is modified in the gene, at least one nucleotide is substituted in the gene, or a combination thereof, relative to the gene in its unmodified state.

286. An engineered T cell comprising:
(a) an effector protein comprising an amino acid sequence that is at least 95% identical to SEQ ID NO: 2435, or a nucleic acid encoding the same; and
(b) a guide nucleic acid, wherein the guide nucleic acid comprises a nucleotide sequence of 5′-AAGGAUGCCAAAC-3′ (nucleotides 57 to 69 of SEQ ID NO: 2522).

287. The engineered T cell of claim 286, wherein the engineered T cell comprises a nucleic acid encoding a chimeric antigen receptor (CAR).

288. The engineered T cell of claim 287, wherein the engineered T cell comprises an mRNA encoding the effector protein.

289. A method of treating a human subject, the method comprising administering to the human subject the engineered T cell of claim 275.

290. A method of modifying a gene of a T cell comprising contacting the T cell with a composition comprising:
(a) an effector protein comprising an amino acid sequence that is at least 95% identical to SEQ ID NO: 2435, or a nucleic acid encoding the same; and
(b) a guide nucleic acid, wherein the guide nucleic acid comprises a nucleotide sequence of 5′-AAGGAUGCCAAAC-3′ (nucleotides 57 to 69 of SEQ ID NO: 2522).

291. The method of claim 290, wherein the T cell is electroporated with: (a) the effector protein, or the nucleic acid encoding the effector protein, and (2) the guide nucleic acid.

292. The method of claim 290, wherein the T cell comprises a nucleic acid encoding a chimeric antigen receptor (CAR).
